(19) United States
(12) Patent Application Publication
(10) Pub. No.: US 2025/0204244 A1
(43) Pub. Date: Jun. 19, 2025

(54) ORGANIC ELECTROLUMINESCENT COMPOUND, A PLURALITY OF HOST MATERIALS, AND ORGANIC ELECTROLUMINESCENT DEVICE COMPRISING THE SAME

(71) Applicant: DuPont Specialty Materials Korea Ltd., Chungcheongnam-do (KR)

(72) Inventors: Hyo-Jung LEE, Gyeonggi-do (KR); So-Young JUNG, Gyeonggi-do (KR); Hyun-Ju KANG, Gyeonggi-do (KR); Sang-Hee CHO, Gyeonggi-do (KR); Mi-Ran SEO, Gyeonggi-do (KR); Ji-Song JUN, Gyeonggi-do (KR)

(21) Appl. No.: 18/950,760

(22) Filed: Nov. 18, 2024

(30) Foreign Application Priority Data
Dec. 15, 2023 (KR) 10-2023-0182920
Oct. 14, 2024 (KR) 10-2024-0139237

(51) Int. Cl. H10K 85/60; H10K 50/12; H10K 85/40; H10K 101/00

(52) U.S. Cl. CPC H10K 85/653; H10K 50/12; H10K 85/40; H10K 85/622; H10K 85/633; H10K 85/654; H10K 85/6572; H10K 85/6574; H10K 85/6576; H10K 2101/90

(57) ABSTRACT

The present disclosure relates to an organic electroluminescent compound, a plurality of host materials, and an organic electroluminescent device comprising the same. It is possible to produce an organic electroluminescent device having improved driving voltage, luminous efficiency, and/or lifetime properties compared to conventional organic electroluminescent device, by comprising the compound or the plurality of host materials according to present disclosure.

### TECHNICAL FIELD

The present disclosure relates to an organic electroluminescent compound, a plurality of host materials, and an organic electroluminescent device comprising the same.

### BACKGROUND ART

In 1987, Tang et al. of Eastman Kodak first developed a small-molecule green organic electroluminescent device (OLED) of TPD/Alq3 bilayer consisting of a light-emitting layer and a charge transport layer. Since then, the research on OLEDs has been rapidly carried out, and OLEDs have been commercialized. At present, phosphorescent materials, which provide excellent luminous efficiency in realizing panels, are mainly used in OLEDs. An OLED which has low driving voltage, high luminous efficiency, and/or long lifetime is required for long time uses and high resolution of displays.

Korean Patent Application Laid-Open No. 2017-0067643 discloses a compound having the following structure.

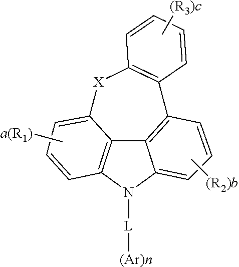

Korean Patent Application Laid-Open No. 2017-0074649 discloses a compound having the following structure.

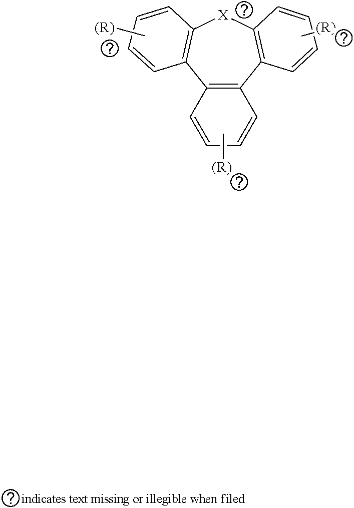

However, the aforementioned references do not specifically disclose the organic electroluminescent compound and a plurality of host materials of the present disclosure.

### DISCLOSURE OF INVENTION

### Technical Problem

The objective of the present disclosure is, firstly, to provide an organic electroluminescent compound and a plurality of host materials capable of producing an organic electroluminescent device having low driving voltage, high luminous efficiency, and/or improved lifetime properties, and secondly, to provide an organic electroluminescent material and an organic electroluminescent device comprising the organic electroluminescent compound or the plurality of host materials.

### Solution to Problem

The present inventors have found that the above objective can be achieved by an organic electroluminescent compound represented by the following Formula 1, and a plurality of host materials comprising a compound represented by the following Formula 1, and a compound represented by the following Formula 4 or 5.

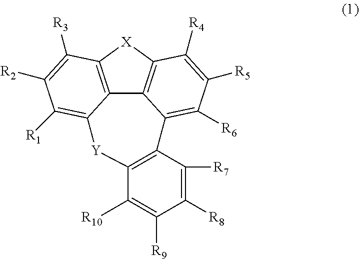

In Formula 1,
X and Y, each independently, represent O or S;
R₁ to R₁₀, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), a substituted or unsubstituted mono- or di-(C1-C30)alkylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, or a substituted or unsubstituted (C6-C30)aryl(3- to 30-membered)heteroarylamino; or may be linked to an adjacent substituent(s) to form a ring(s); with the proviso that R₆ and R₇ are not linked to each other to form a ring(s);
with the proviso that at least one of R₁ to R₁₀ is represented by the following Formula 1-1 or 1-2:

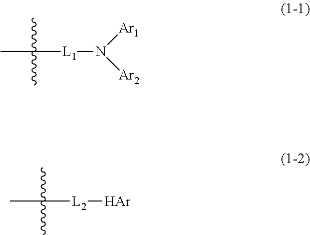

wherein in Formulas 1-1 and 1-2,
L₁ and L₂, each independently, represent a single bond, a substituted or unsubstituted (C6-C30)arylene, or a substituted or unsubstituted (3- to 30-membered)heteroarylene;

Ar₁ and Ar₂, each independently, represent hydrogen, deuterium, a substituted or unsubstituted (C6-C30)aryl, or a substituted or unsubstituted (3- to 30-membered)heteroaryl; and
HAr represents a substituted or unsubstituted (3- to 30-membered)heteroaryl containing one or more nitrogen atoms.

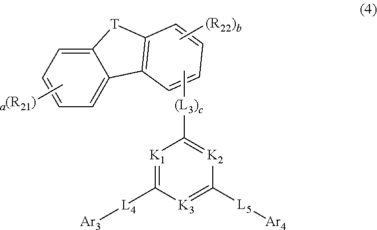

In Formula 4,
T represents O or S;
K₁ to K₃, each independently, represent N or CR₂₃, with the proviso that at least one of K₁ to K₃ represents N;
L₃ to L₅, each independently, represent a single bond, a substituted or unsubstituted (C6-C30)arylene, a substituted or unsubstituted (C3-C30)cycloalkylene, or a substituted or unsubstituted (3- to 30-membered)heteroarylene;
R₂₁ to R₂₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), or —N—(R′)(R″); or may be linked to an adjacent substituent(s) to form a ring(s);
Ar₃ and Ar₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), or —N—(R′)(R″); or may be linked to an adjacent substituent(s) to form a ring(s);
R′ and R″, each independently, represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; and
a represents an integer of 1 to 4, b represents an integer of 1 to 3, c represents an integer of 1 or 2, and if a to c, respectively, represent an integer of 2 or more, each of R₂₁, each of R₂₂, and each of L₃ may be the same as or different from each other.

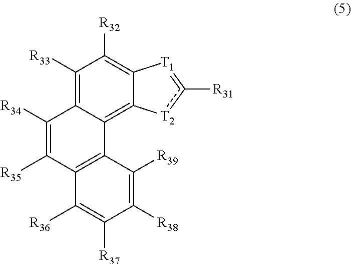

In Formula 5,
T₁ and T₂, each independently, represent —N═, —NR₄₀—, —O—, or —S—, with the proviso that any one of T₁ and T₂ represents —N═, and the other of T₁ and T₂ represents —NR₄₀—, —O—, or —S—;
R₃₁ represents a substituted or unsubstituted (C6-C30)aryl, or a substituted or unsubstituted (3- to 30-membered)heteroaryl;
R₃₂ to R₄₀, each independently, are represented by the following Formula 5-1, or represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di-(C6-C30)arylamino, a substituted or unsubstituted mono- or di-(3- to 30-membered)heteroarylamino, or a substituted or unsubstituted (C6-C30)aryl(3- to 30-membered)heteroarylamino; or may be linked to an adjacent substituent(s) to form a ring(s);
with the proviso that at least one of R₃₂ to R₃₉ is represented by the following Formula 5-1:

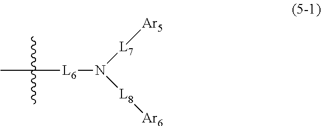

wherein in Formula 5-1,
L₆ to L₈, each independently, represent a single bond, a substituted or unsubstituted (C6-C30)arylene, or a substituted or unsubstituted (3- to 30-membered)heteroarylene; and
Ar₅ and Ar₆, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted (C6-C30)aryl(3- to 30-membered)heteroarylamino, or a substituted or unsubstituted fused ring group of a (C3-C30)aliphatic ring(s) and a (C6-C30)aromatic ring(s), or may be linked to an adjacent substituent(s) to form a ring(s).

### Advantageous Effects of Invention

An organic electroluminescent compound and a plurality of host materials according to the present disclosure can provide an organic electroluminescent device having low driving voltage, high luminous efficiency, and/or improved lifetime properties. In particular, an organic electroluminescent device comprising an organic electroluminescent compound or a plurality of host materials according to the present disclosure can exhibit long lifetime properties while having low driving voltage and/or high luminous efficiency.

### MODE FOR THE INVENTION

Hereinafter, the present disclosure will be described in detail. However, the following description is intended to explain the present disclosure, and is not meant in any way to restrict the scope of the present disclosure.

The present disclosure relates to an organic electroluminescent compound represented by Formula 1, an organic electroluminescent material comprising the organic electroluminescent compound, a plurality of host materials comprising a compound represented by Formula 1 and a compound represented by Formula 4 or 5, and an organic electroluminescent device comprising the organic electroluminescent compound, the organic electroluminescent material, and/or the plurality of host materials.

The term “organic electroluminescent compound” in the present disclosure means a compound that may be used in an organic electroluminescent device, and may be comprised in any layer constituting an organic electroluminescent device, as necessary.

The term “an organic electroluminescent material” in the present disclosure means a material that may be used in an organic electroluminescent device, and may comprise at least one compound. If necessary, the organic electroluminescent material may be comprised in any layer constituting an organic electroluminescent device. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of host materials” in the present disclosure means a host material comprising a combination of at least two compounds, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, the plurality of host materials of the present disclosure is a combination of at least two host materials, and may selectively further comprise conventional materials comprised in an organic electroluminescent material. At least two compounds comprised in the plurality of host materials of the present disclosure may be comprised together in one light-emitting layer or may each be comprised in different light-emitting layers. For example, the at least two host materials may be mixture-evaporated or co-evaporated, or may be individually evaporated.

Herein, the “(C1-C30)alkyl” is meant to be a linear or branched alkyl having 1 to 30 carbon atoms constituting a chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. Herein, the “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. Herein, the “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. Herein, the “(C3-C30)cycloalkyl” is meant to be a mono- or polycyclic hydrocarbon having 3 to 30 ring backbone carbon atoms, in which the number of ring backbone carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. Herein, the term “(3- to 7-membered)heterocycloalkyl” is meant to be a cycloalkyl having 3 to 7 ring backbone atoms, preferably 5 to 7 ring backbone atoms, and containing at least one heteroatom selected from the group consisting of B, N, O, S, Si, P, Te, Se, and Ge, preferably at least one heteroatoms selected from the group consisting of O, S, N, and Se. The above heterocycloalkyl may include tetrahydrofuran, pyrrolidine, thiolan, tetrahydropyran, etc. Herein, the “(C6-C30)aryl” or “(C6-C30)arylene” is meant to be a monocyclic or fused ring-type radical derived from an aromatic hydrocarbon having 6 to 30 ring backbone carbon atoms that can be partially saturated. The above aryl may comprise a spiro structure. The above aryl may include phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, phenylphenanthrenyl, benzophenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluoren]yl, spiro[cyclopentene-fluoren]yl, spiro[dihydroindene-fluoren]yl, azulenyl, tetramethyl-dihydrophenanthrenyl, etc. Specifically, the above aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, o-quaterphenyl, m-quaterphenyl, p-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, 0-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

Herein, the “(3- to 30-membered)heteroaryl” or “(3- to 30-membered)heteroarylene” is meant to be an aryl or arylene group having 3 to 30 ring backbone atoms, and comprising at least one heteroatom selected from the group consisting of B, N, O, S, Si, P, Te, Se, and Ge. Preferably, the number of the heteroatoms may be 1 to 4. The above heteroaryl or heteroarylene may be a monocyclic ring, or a fused ring condensed with at least one benzene ring; may be partially saturated; may comprise one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, dibenzothiophenyl, dibenzoselenophenyl, naphthobenzofuranyl, benzofuroquinolinyl, benzofuroquinazolinyl, naphthobenzothiophenyl, benzofuronaphthyridinyl, benzofuropyridyl, benzofuropyrimidinyl, naphthofuropyrimidinyl, benzothienoquinolyl, benzothienoquinazolinyl, naphthyridinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, benzoquinazolinyl, quinoxalinyl, benzoquinoxalinyl, carbazolyl, benzocarbazolyl, dibenzocarbazolyl, phenoxazinyl, phenanthridinyl, benzodioxolyl, dehydroacridinyl, benzotriazolyl, phenazinyl, imidazopyridyl, chromenoquinazolinyl, thiochromenoquinazolinyl, dimethylbenzopyrimidinyl, indolocarbazolyl, indenocarbazolyl, etc. More specifically, the above heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl) pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. In addition, “heteroaryl(ene)” may be classified into heteroaryl(ene) with electronic properties and heteroaryl(ene) with hole properties. Heteroaryl(ene) with electronic properties is a substituent that is relatively rich in electrons in the parent nucleus, for example, a substituted or unsubstituted pyridinyl, a substituted or unsubstituted pyrimidinyl, a substituted or unsubstituted triazinyl, a substituted or unsubstituted quinazolinyl, a substituted or unsubstituted, a substituted d or unsubstituted quinolyl, etc. Heteroaryl(ene) with hole properties is a substituent that is relatively electron-deficient in the parent nucleus, for example, a substituted or unsubstituted carbazolyl, a substituted or unsubstituted dibenzofuranyl, a substituted or unsubstituted dibenzothiophenyl, etc.

Herein, “a fused ring group of a (C3-C30)aliphatic ring(s) and a (C6-C30)aromatic ring(s)” is meant to be a functional group of a ring in which at least one aliphatic ring having 3 to 30 ring backbone carbon atoms, preferably 3 to 25 ring backbone carbon atoms, and more preferably 3 to 18 ring backbone carbon atoms, is fused with at least one aromatic ring having 6 to 30 ring backbone carbon atoms, preferably 6 to 25 ring backbone carbon atoms, and more preferably 6 to 18 ring backbone carbon atoms. Specific examples of the fused ring group include a fused ring group of one or more benzene and one or more cyclohexane, or a fused ring group of one or more naphthalene and one or more cyclopentane, etc. Herein, the carbon atom of the fused ring group of a (C3-C30)aliphatic ring(s) and a (C6-C30)aromatic ring(s) may be replaced with one or more heteroatoms selected from B, N, O, S, Si, P, Te, Se, and Ge, preferably one or more heteroatoms selected from N, O, S, and Se. Herein, “halogen” includes F, Cl, Br, and I.

In addition, “ortho-” (“o-”), “meta-” (“m-”), and “para-” (“p-”) are each prefixes which represent the relative positions of substituents. The prefix “ortho-” indicates that two substituents are adjacent to each other; for example, when two substituents in a benzene derivative occupy positions 1 and 2, it is called an “ortho-” position. The prefix “meta-” indicates that two substituents are at positions 1 and 3; for example, when two substituents in a benzene derivative occupy positions 1 and 3, it is called a “meta-” position. The prefix “para-” indicates that two substituents are at positions 1 and 4; for example, when two substituents in a benzene derivative occupy positions 1 and 4, it is called a “para-” position.

Herein, “a ring formed by being linked to an adjacent substituent(s)” means that at least two adjacent substituents are linked to or fused with each other to form a substituted or unsubstituted, mono- or polycyclic, (3- to 30-membered) alicyclic or aromatic ring, or a combination thereof. The ring may also form a spiro ring. Preferably, the ring may be a substituted or unsubstituted, mono- or polycyclic, (3- to 26-membered) alicyclic or aromatic ring, or a combination thereof. More preferably, the ring may be a mono- or polycyclic (5- to 25-membered)aromatic ring unsubstituted or substituted with at least one of a (C₁-C₆)alkyl(s), a (C6-C18)aryl(s), and a (3- to 20-membered)heteroaryl(s). In addition, the ring may contain at least one heteroatom selected from B, N, O, S, Si, P, Te, Se, and Ge, preferably at least one heteroatom selected from N, O, S, and Se. For example, the ring may be a benzene ring, etc.

Herein, heteroaryl, heteroarylene, and heterocycloalkyl, each independently, may contain at least one heteroatom selected from the group consisting of B, N, O, S, Si, P, Te, and Se. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, and a substituted or unsubstituted (C6-C30)aryl(3- to 30-membered)heteroarylamino.

Herein, “substituted” in the expression “substituted or unsubstituted” means that a hydrogen atom in a certain functional group is replaced with another atom or another functional group, i.e., a substituent. Unless otherwise specified, the substituent may replace hydrogen at a position where the substituent can be substituted without limitation, and when two or more hydrogen atoms in a certain functional group are each replaced with a substituent, each substituent may be the same as or different from each other. The maximum number of substituents that can be substituted for a certain functional group may be the total number of valences that can be substituted for each atom forming the functional group. Herein, the substituted alkyl, the substituted aryl, the substituted arylene, the substituted heteroaryl, the substituted heteroarylene, the substituted cycloalkyl, the substituted alkoxy, the substituted trialkylsilyl, the substituted dialkylarylsilyl, the substituted alkyldiarylsilyl, the substituted triarylsilyl, the substituted fused ring group of an aliphatic ring(s) and an aromatic ring(s), the substituted mono- or di-alkylamino, mono- or di-alkenylamino, the substituted alkylalkenylamino, the substituted alkenylheteroarylamino, the substituted mono- or di-arylamino, the substituted alkylarylamino, the substituted mono- or di-heteroarylamino, and the substituted arylheteroarylamino, each independently, is substituted with at least one selected from the group consisting of deuterium, a halogen, a cyano, a carboxyl, a nitro, a hydroxyl, a (C1-C30)alkyl, a halo(C1-C30)alkyl, a (C2-C30)alkenyl, a (C2-C30)alkynyl, a (C1-C30)alkoxy, a (C1-C30)alkylthio, a (C3-C30)cycloalkyl, a (C3-C30)cycloalkenyl, a (3- to 7-membered)heterocycloalkyl, a (C6-C30)aryloxy, a (C6-C30)arylthio, a (3- to 30-membered)heteroaryl, a (C6-C30)aryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, a fused ring group of a (C3-C30)aliphatic ring(s) and a (C6-C30) aromatic ring(s), an amino, a mono- or di-(C1-C30)alkylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C6-C30)aryl(3- to 30-membered)heteroarylamino, a (C1-C30)alkylcarbonyl, a (C1-C30)alkoxycarbonyl, a (C6-C30)arylcarbonyl, a (C6-C30)arylphosphinyl, a di(C6-C30)arylboronyl, a di(C1-C30)alkylboronyl, a (C1-C30)alkyl(C6-C30)arylboronyl, a (C6-C30)aryl(C1-C30)alkyl, a (C1-C30)alkyl(C6-C30)aryl, and a combination thereof. According to one embodiment of the present disclosure, the group may consist of deuterium, a (C1-C20)alkyl, a (C6-C25)aryl, and a (5- to 25-membered)heteroaryl. According to one embodiment of the present disclosure, the group may consist of deuterium, a (C1-C10)alkyl, a (C6-C24)aryl, and a (6- to 22-membered)heteroaryl. For example, the group may consist of deuterium, methyl, tert-butyl, phenyl, biphenyl, naphthyl, dimethylfluorenyl, phenanthrenyl, chrysenyl, terphenyl, triphenylenyl, quaterphenyl, pyridyl, dibenzofuranyl, dibenzothiophenyl, carbazolyl, dibenzoselenophenyl, phenanthrofuranyl, benzonaphthofuranyl, benzonaphthothiophenyl, benzonaphthoselenophenyl, benzophenanthrofuranyl, diphenylamino, triphenylsilyl, etc.

Herein, if a substituent is not indicated in the Formula or compound structure, it may mean that all possible positions for the substituent are hydrogen or deuterium. That is, in the case of deuterium, it is an isotope of hydrogen, and some hydrogen atoms may be the isotope deuterium, and in this case, the content of deuterium may be 0% to 100%. In cases where a substituent is not indicated in the formula or compound structure in the present disclosure, if the substituent is not explicitly excluded, such as 0% deuterium, 100% hydrogen, and all substituents being hydrogen, hydrogen and deuterium may be used intermixed in a compound. The deuterium is one of the isotopes of hydrogen and an element with a deuteron consisting of one proton and one neutron as its nucleus. It can be represented as hydrogen-2, whose element symbol can also be written as D or 2H. Isotopes are atoms with the same atomic number (Z) but different mass numbers (A), and can also be interpreted as elements with the same number of protons but different numbers of neutrons.

Herein, “a combination thereof” refers to a combination of one or more elements from the corresponding list to form a known or chemically stable arrangement that can be envisioned by one skilled in the art from the corresponding list. For example, alkyl and deuterium can be combined to form a partially or fully deuterated alkyl group; and halogen and alkyl can be combined to form a halogenated alkyl substituent; halogen, alkyl, and aryl can be combined to form a halogenated arylalkyl. For example, a preferred combination of substituents includes up to 50 atoms that are not hydrogen or deuterium, up to 40 atoms that are not hydrogen or deuterium, or up to 30 atoms that are not hydrogen or deuterium, but in many cases, a preferred combination of substituents may comprise up to 20 atoms that are not hydrogen or deuterium.

In the formulas of the present disclosure, when there are multiple substituents represented by the same symbol, each substituent represented by the same symbol may be the same as or different from each other.

In Formula 1, X and Y, each independently, represent O or S. According to one embodiment of the present disclosure, X and Y each represent O.

In Formula 1, R₁ to R₁₀, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), a substituted or unsubstituted mono- or di-(C1-C30)alkylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, or a substituted or unsubstituted (C6-C30)aryl(3- to 30-membered)heteroarylamino; or may be linked to an adjacent substituent(s) to form a ring(s); with the proviso that R₆ and R₇ are not linked to each other to form a ring(s), and at least one of R₁ to R₁₀ is represented by Formula 1-1 or 1-2. According to one embodiment of the present disclosure, R₁ to R₁₀, each independently, represent hydrogen, or a substituted or unsubstituted (C6-C25)aryl, or are represented by Formula 1-1 or 1-2. According to another embodiment of the present disclosure, R₁ to R₁₀, each independently, represent hydrogen, or an unsubstituted (C6-C18)aryl, or are represented by Formula 1-1 or 1-2. According to one embodiment of the present disclosure, any one of R₁ to R₁₀ is represented by Formula 1-1 or 1-2.

In Formulas 1-1 and 1-2, L₁ and L₂, each independently, represent a single bond, a substituted or unsubstituted (C6-C30)arylene, or a substituted or unsubstituted (3- to 30-membered)heteroarylene. According to one embodiment of the present disclosure, L₁ and L₂, each independently, represent a single bond, or a substituted or unsubstituted (C6-C25)arylene. According to another embodiment of the present disclosure, L₁ and L₂, each independently, represent a single bond, or an unsubstituted (C6-C18)arylene. For example, L₁ and L₂, each independently, may be a single bond, a phenylene, a naphthylene, etc.

In Formula 1-1, Ar₁ and Ar₂, each independently, represent hydrogen, deuterium, a substituted or unsubstituted (C6-C30)aryl, or a substituted or unsubstituted (3- to 30-membered)heteroaryl. According to one embodiment of the present disclosure, Ar₁ and Ar₂, each independently, represent a substituted or unsubstituted (C6-C25)aryl, or a substituted or unsubstituted (5- to 25-membered)heteroaryl. According to another embodiment of the present disclosure, Ar₁ and Ar₂, each independently, represent a substituted or unsubstituted (C6-C18)aryl, or a substituted or unsubstituted (5- to 20-membered)heteroaryl. According to one embodiment of the present disclosure, the aryl or the heteroaryl may be substituted with at least one selected from the group consisting of a (C1-C30)alkyl(s), a (C6-C30)aryl(s), a (3- to 30-membered)heteroaryl(s), and a combination thereof. For example, Ar₁ and Ar₂, each independently, may be a phenyl, a naphthyl, a biphenyl, a phenanthrenyl, a dimethylfluorenyl, a terphenyl, a chrysenyl, a dibenzofuranyl, a dibenzothiophenyl, a carbazolyl, etc., which may be substituted with at least one selected from the group consisting of a phenyl, a naphthyl, a biphenyl, a phenanthrenyl, a dibenzofuranyl, a dibenzothiophenyl, a benzonaphthofuranyl, and a combination thereof.

In Formula 1-2, HAr represents a substituted or unsubstituted (3- to 30-membered)heteroaryl containing one or more nitrogen atoms. According to one embodiment of the present disclosure, HAr may contain two or more nitrogen atoms. According to one embodiment of the present disclosure, HAr represents a substituted (5- to 25-membered)heteroaryl, or a substituted (5- to 20-membered)heteroaryl. According to one embodiment of the present disclosure, the heteroaryl may be substituted with at least one selected from the group consisting of a (C1-C30)alkyl(s), a (C6-C30)aryl(s), a (3- to 30-membered)heteroaryl(s), and a combination thereof. For example, HAr may be a triazinyl, a quinoxalinyl, a quinazolinyl, a benzoquinoxalinyl, a benzoquinazolinyl, etc., which may be substituted with at least one selected from the group consisting of a phenyl, a naphthyl, a biphenyl, a phenanthrenyl, a chrysenyl, a terphenyl, a triphenylenyl, a dimethylfluorenyl, a quaterphenyl, a pyridyl, a dibenzofuranyl, a dibenzothiophenyl, a carbazolyl, a dibenzoselenophenyl, a phenanthrofuranyl, a benzonaphthothiophenyl, a benzonaphthofuranyl, a benzonaphthoselenophenyl, a benzophenanthrofuranyl, and a combination thereof.

According to one embodiment of the present disclosure, HAr may be represented by the following Formula 1-a or 1-b.

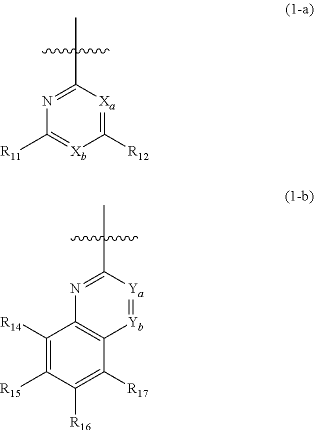

In Formulas 1-a and 1-b, Xₐ, X_{b}, Yₐ, and Y_{b}, each independently, represent N or CR₁₃, with the proviso that at least one of Xₐ and X_{b} represents N, and at least one of Yₐ and Y_{b} represents N. According to one embodiment of the present disclosure, Xₐ and X_{b} each represent N. According to one embodiment of the present disclosure, any one of Yₐ and Y_{b} represents N, and the other represents CR₁₃.

In Formulas 1-a and 1-b, R₁₁ to R₁₇, each independently, represent hydrogen, deuterium, a halogen, a cyano, a substituted or unsubstituted (C1-C30)alkyl, a substituted or unsubstituted (C6-C50)aryl, a substituted or unsubstituted (3- to 50-membered)heteroaryl, a substituted or unsubstituted (C3-C30)cycloalkyl, a substituted or unsubstituted (3- to 7-membered)heterocycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent(s) to form a ring(s).

According to one embodiment of the present disclosure, R₁₁ and R₁₂, each independently, represent a substituted or unsubstituted (C6-C30)aryl, or a substituted or unsubstituted (3- to 30-membered)heteroaryl. According to another embodiment of the present disclosure, R₁₁ and R₁₂, each independently, represent a substituted or unsubstituted (C6-C25)aryl, or a substituted or unsubstituted (5- to 25-membered)heteroaryl. According to one embodiment of the present disclosure, the aryl or the heteroaryl may be substituted with at least one selected from the group consisting of a (C1-C30)alkyl(s), a (C6-C30)aryl(s), a (3- to 30-membered)heteroaryl(s), and a combination thereof. For example, R₁₁ and R₁₂, each independently, may be a phenyl, a naphthyl, a biphenyl, a phenanthrenyl, a chrysenyl, a terphenyl, a triphenylenyl, a dimethylfluorenyl, a quaterphenyl, a dibenzofuranyl, a dibenzothiophenyl, a dibenzoselenophenyl, a carbazolyl, a phenanthrofuranyl, a benzonaphthothiophenyl, a benzonaphthofuranyl, a benzonaphthoselenophenyl, a benzophenanthrofuranyl, etc., which may be substituted with at least one selected from the group consisting of methyl, phenyl, naphthyl, terphenyl, pyridyl, dibenzofuranyl, carbazolyl, and a combination thereof.

According to one embodiment of the present disclosure, R₁₃ represents a substituted or unsubstituted (C6-C30)aryl, or a substituted or unsubstituted (3- to 30-membered)heteroaryl. According to another embodiment of the present disclosure, R₁₃ represents an unsubstituted (C6-C25)aryl, or an unsubstituted (5- to 25-membered)heteroaryl. For example, R₁₃ may be a phenyl, a naphthyl, a dibenzofuranyl, etc.

According to one embodiment of the present disclosure, R₁₄ to R₁₇, each independently, represent hydrogen, a substituted or unsubstituted (C6-C30)aryl, or a substituted or unsubstituted (3- to 30-membered)heteroaryl; or may be linked to an adjacent substituent(s) to form a substituted or unsubstituted, mono- or polycyclic, (3- to 30-membered) alicyclic or aromatic ring, or a combination thereof. According to another embodiment of the present disclosure, R₁₄ to R₁₇, each independently, represent hydrogen; or may be linked to an adjacent substituent(s) to form a substituted or unsubstituted monocyclic (3- to 30-membered)aromatic ring. For example, R₁₄ to R₁₇, each independently, may be hydrogen; or may be linked to an adjacent substituent to form a benzene ring.

Formula 1 may be represented by at least one of the following Formulas 2-1 to 2-10.

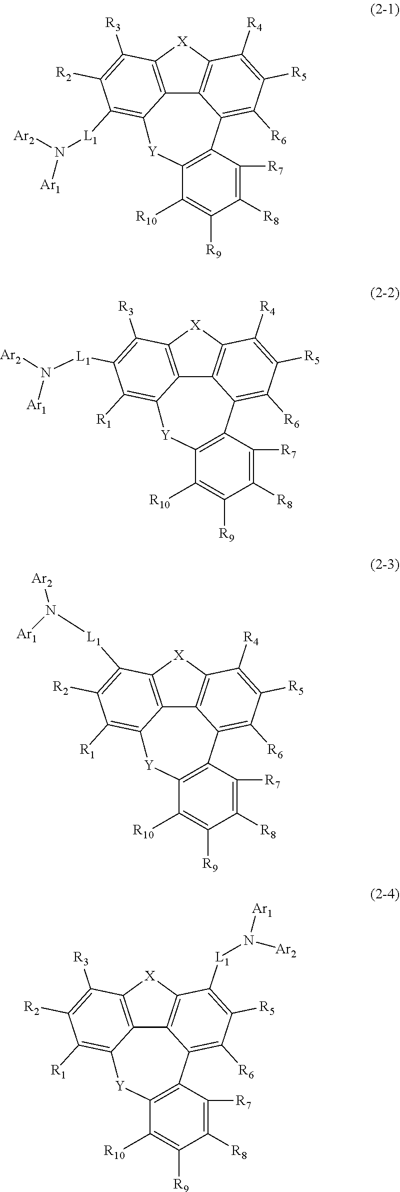

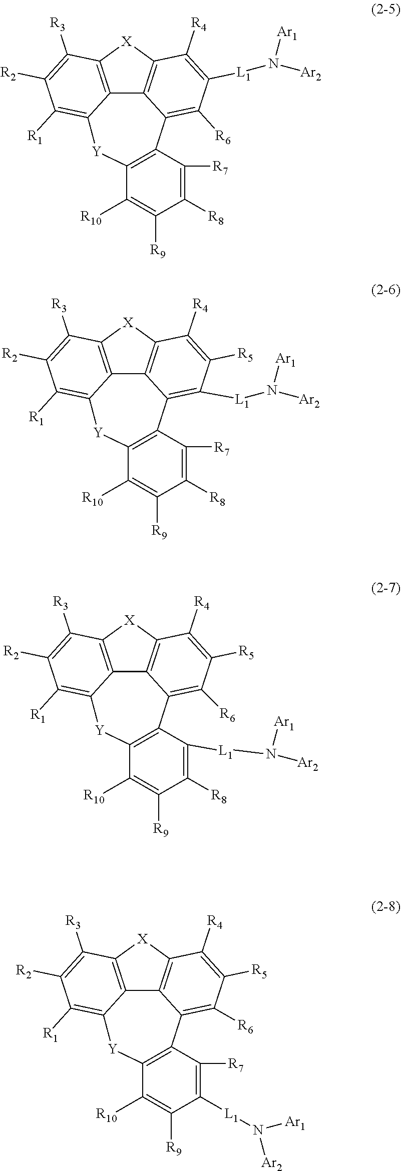

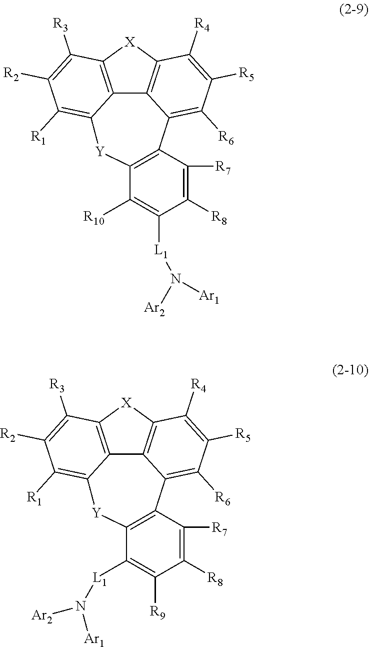

In Formulas 2-1 to 2-10, X, Y, R₁ to R₁₀, L₁, Ar₁, and Ar₂ are as defined in Formula 1.

Formula 1 may be represented by at least one of the following Formulas 3-1 to 3-10.

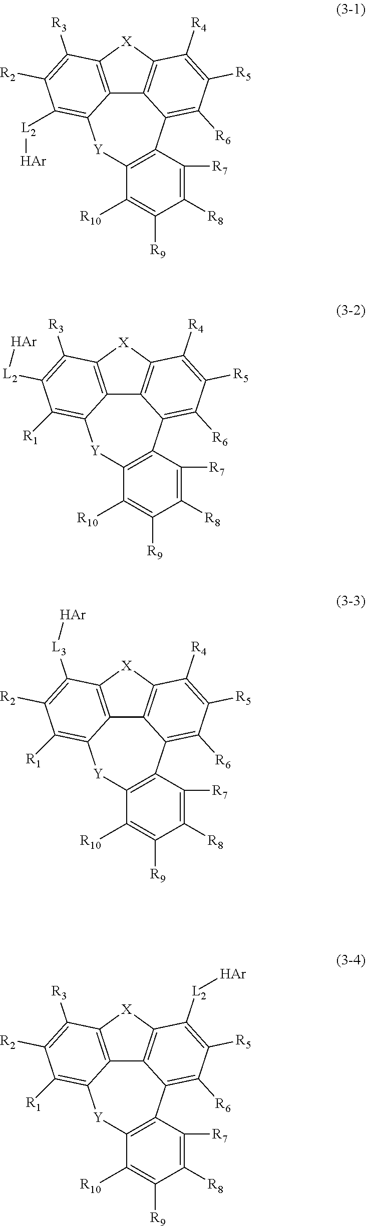

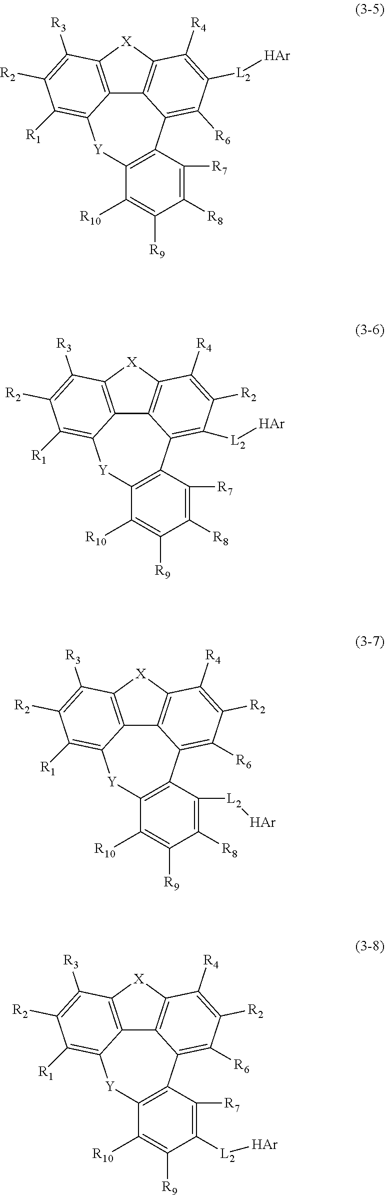

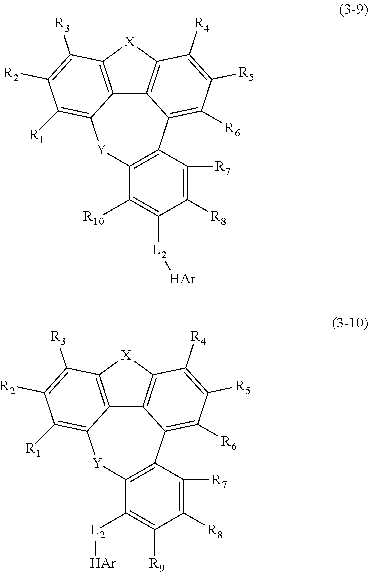

In Formulas 3-1 to 3-10, X, Y, R₁ to R₁₀, L₂, and HAr are as defined in Formula 1.

The compound represented by Formula 1 may be at least one selected from the group consisting of the following compounds, but is not limited thereto.

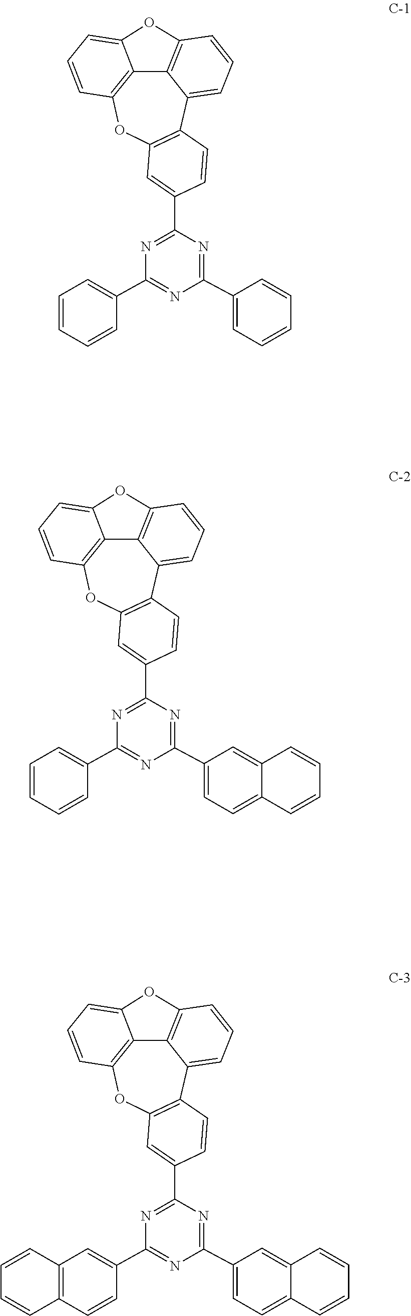 [SMILES: c1ccc(-c2nc(-c3ccccc3)nc(-c3ccc4c(c3)oc3cccc5oc6cccc4c6c35)n2)cc1 ; c1ccc(-c2nc(-c3ccc4ccccc4c3)nc(-c3ccc4c(c3)oc3cccc5oc6cccc4c6c35)n2)cc1 ; c1ccc2cc(-c3nc(-c4ccc5ccccc5c4)nc(-c4ccc5c(c4)oc4cccc6oc7cccc5c7c46)n3)ccc2c1]

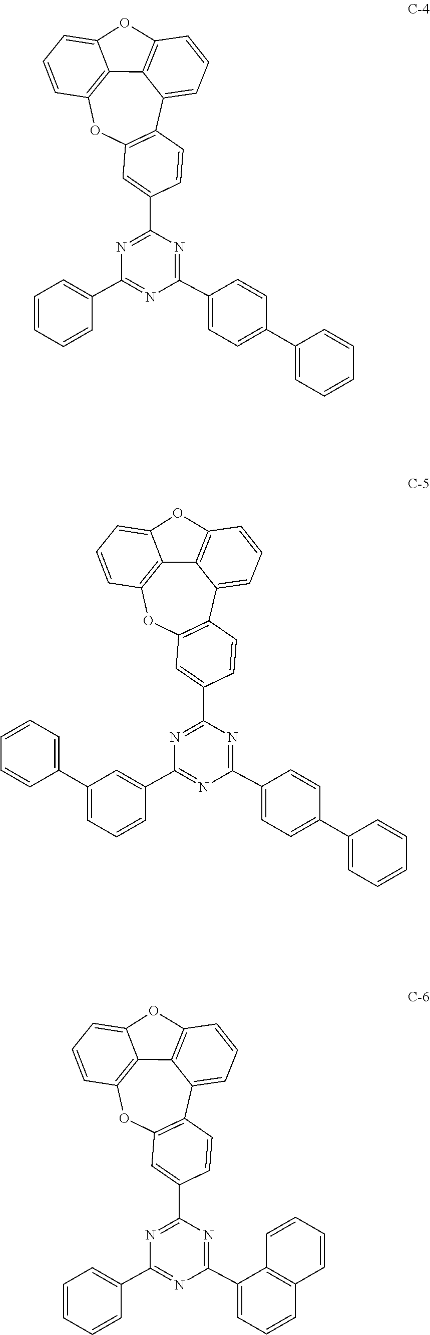 [SMILES: c1ccc(-c2ccc(-c3nc(-c4ccccc4)nc(-c4ccc5c(c4)oc4cccc6oc7cccc5c7c46)n3)cc2)cc1 ; c1ccc(-c2ccc(-c3nc(-c4cccc(-c5ccccc5)c4)nc(-c4ccc5c(c4)oc4cccc6oc7cccc5c7c46)n3)cc2)cc1 ; c1ccc(-c2nc(-c3ccc4c(c3)oc3cccc5oc6cccc4c6c35)nc(-c3cccc4ccccc34)n2)cc1]

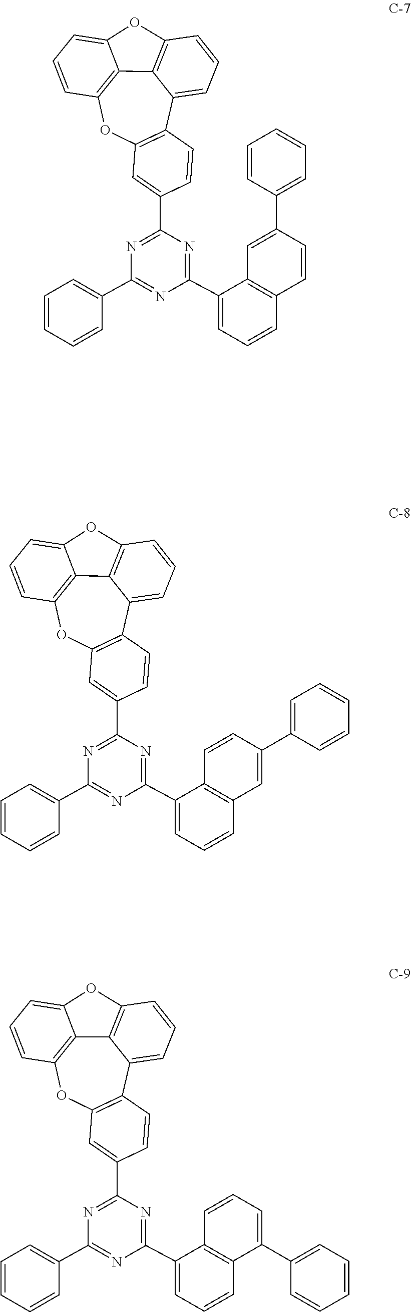 [SMILES: c1ccc(-c2ccc3cccc(-c4nc(-c5ccccc5)nc(-c5ccc6c(c5)oc5cccc7oc8cccc6c8c57)n4)c3c2)cc1 ; c1ccc(-c2ccc3c(-c4nc(-c5ccccc5)nc(-c5ccc6c(c5)oc5cccc7oc8cccc6c8c57)n4)cccc3c2)cc1 ; c1ccc(-c2nc(-c3ccc4c(c3)oc3cccc5oc6cccc4c6c35)nc(-c3cccc4c(-c5ccccc5)cccc34)n2)cc1]

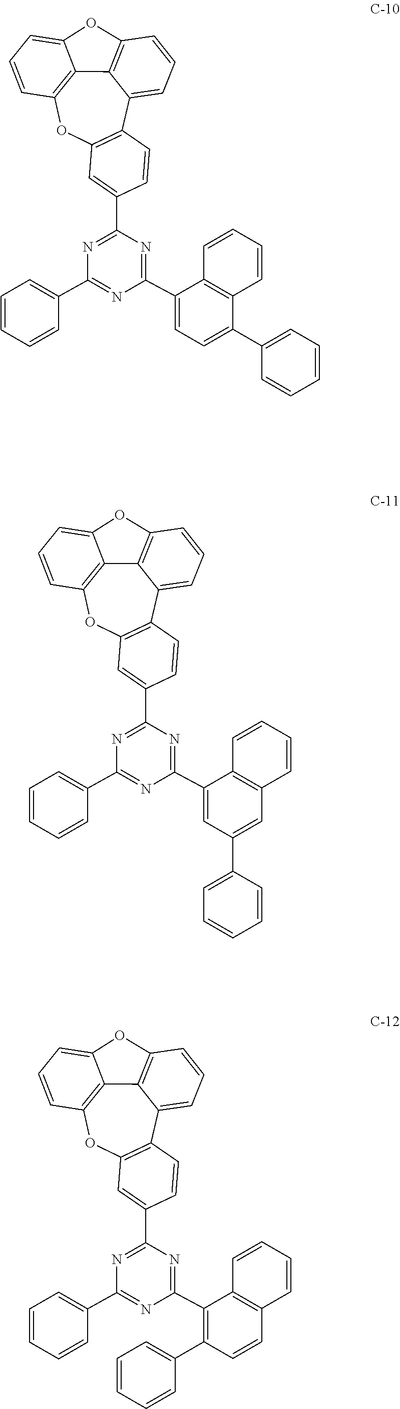 [SMILES: c1ccc(-c2nc(-c3ccc4c(c3)oc3cccc5oc6cccc4c6c35)nc(-c3ccc(-c4ccccc4)c4ccccc34)n2)cc1 ; c1ccc(-c2cc(-c3nc(-c4ccccc4)nc(-c4ccc5c(c4)oc4cccc6oc7cccc5c7c46)n3)c3ccccc3c2)cc1 ; c1ccc(-c2nc(-c3ccc4c(c3)oc3cccc5oc6cccc4c6c35)nc(-c3c(-c4ccccc4)ccc4ccccc34)n2)cc1]

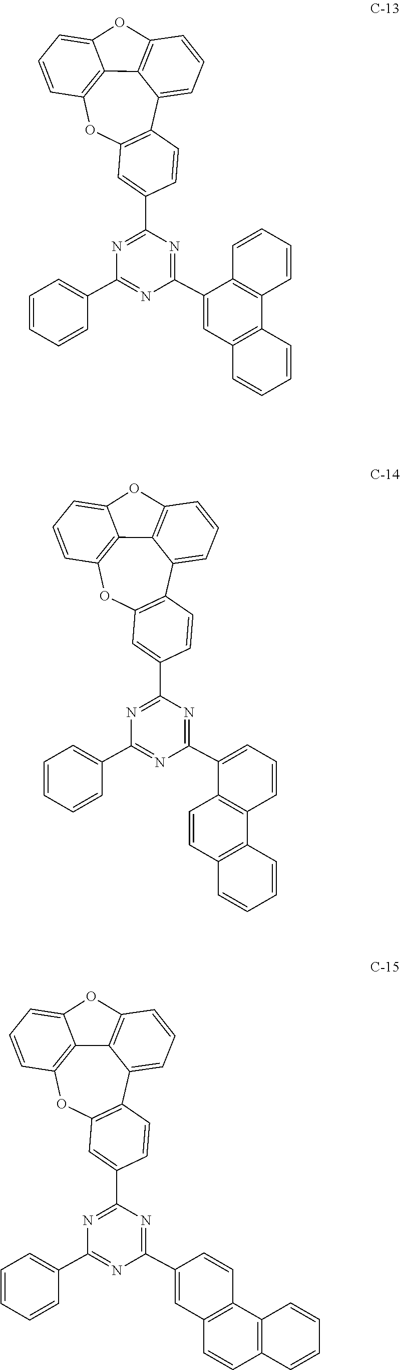 [SMILES: c1ccc(-c2nc(-c3ccc4c(c3)oc3cccc5oc6cccc4c6c35)nc(-c3cc4ccccc4c4ccccc34)n2)cc1 ; c1ccc(-c2nc(-c3ccc4c(c3)oc3cccc5oc6cccc4c6c35)nc(-c3cccc4c3ccc3ccccc34)n2)cc1 ; c1ccc(-c2nc(-c3ccc4c(ccc5ccccc54)c3)nc(-c3ccc4c(c3)oc3cccc5oc6cccc4c6c35)n2)cc1]

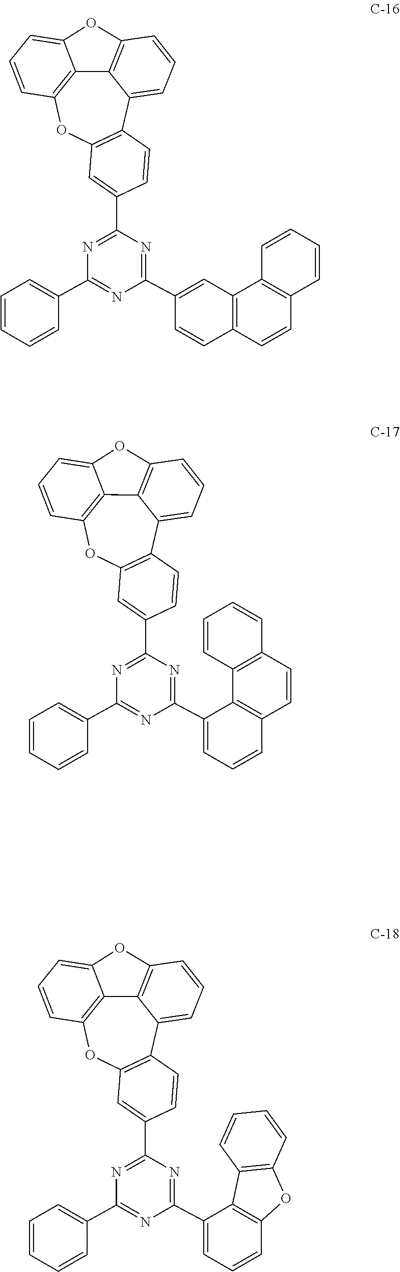 [SMILES: c1ccc(-c2nc(-c3ccc4c(c3)oc3cccc5oc6cccc4c6c35)nc(-c3ccc4ccc5ccccc5c4c3)n2)cc1 ; c1ccc(-c2nc(-c3ccc4c(c3)oc3cccc5oc6cccc4c6c35)nc(-c3cccc4ccc5ccccc5c34)n2)cc1 ; c1ccc(-c2nc(-c3ccc4c(c3)oc3cccc5oc6cccc4c6c35)nc(-c3cccc4oc5ccccc5c34)n2)cc1]

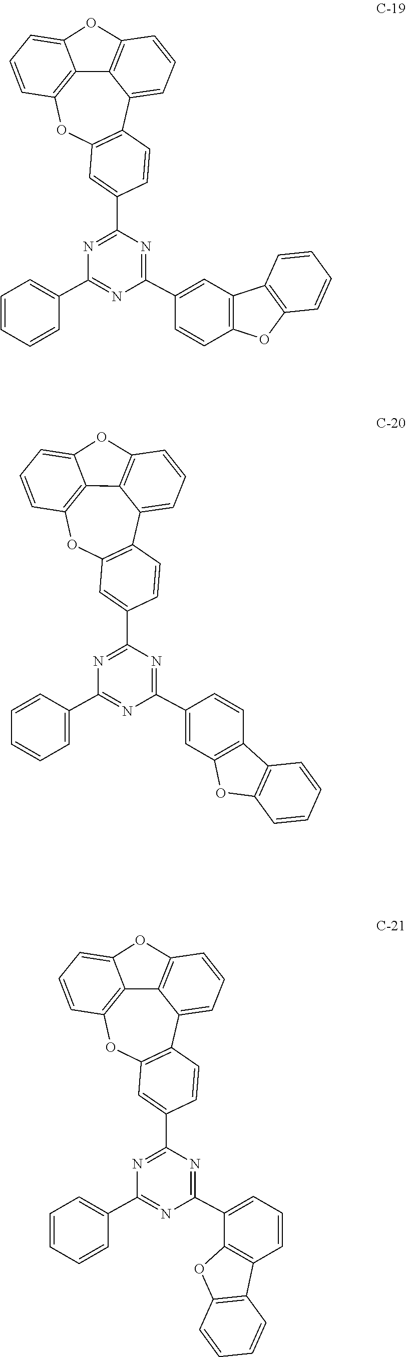 [SMILES: c1ccc(-c2nc(-c3ccc4c(c3)oc3cccc5oc6cccc4c6c35)nc(-c3ccc4oc5ccccc5c4c3)n2)cc1 ; c1ccc(-c2nc(-c3ccc4c(c3)oc3ccccc34)nc(-c3ccc4c(c3)oc3cccc5oc6cccc4c6c35)n2)cc1 ; c1ccc(-c2nc(-c3ccc4c(c3)oc3cccc5oc6cccc4c6c35)nc(-c3cccc4c3oc3ccccc34)n2)cc1]

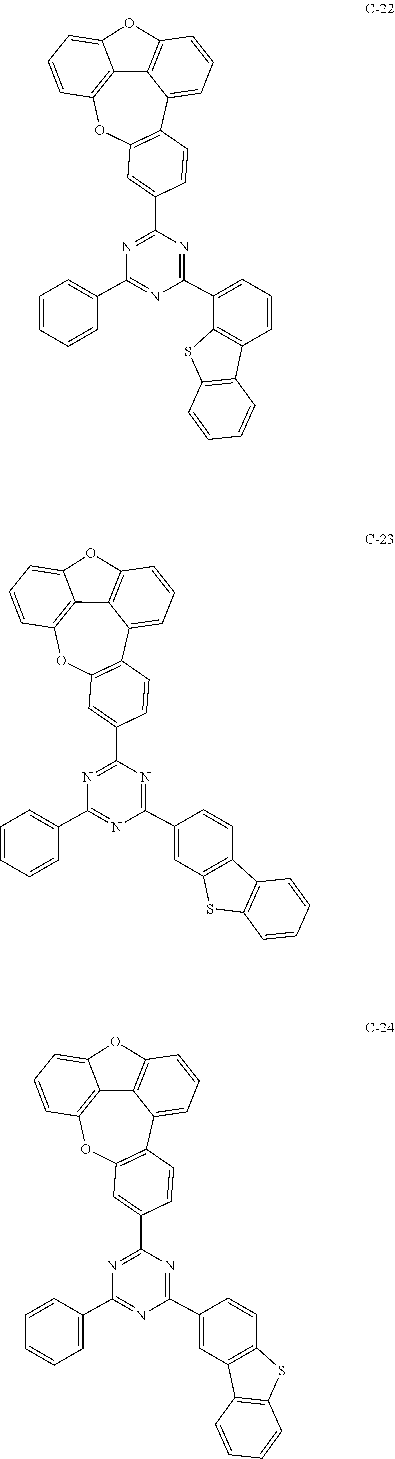 [SMILES: c1ccc(-c2nc(-c3ccc4c(c3)oc3cccc5oc6cccc4c6c35)nc(-c3cccc4c3sc3ccccc34)n2)cc1 ; c1ccc(-c2nc(-c3ccc4c(c3)oc3cccc5oc6cccc4c6c35)nc(-c3ccc4c(c3)sc3ccccc34)n2)cc1 ; c1ccc(-c2nc(-c3ccc4c(c3)oc3cccc5oc6cccc4c6c35)nc(-c3ccc4sc5ccccc5c4c3)n2)cc1]

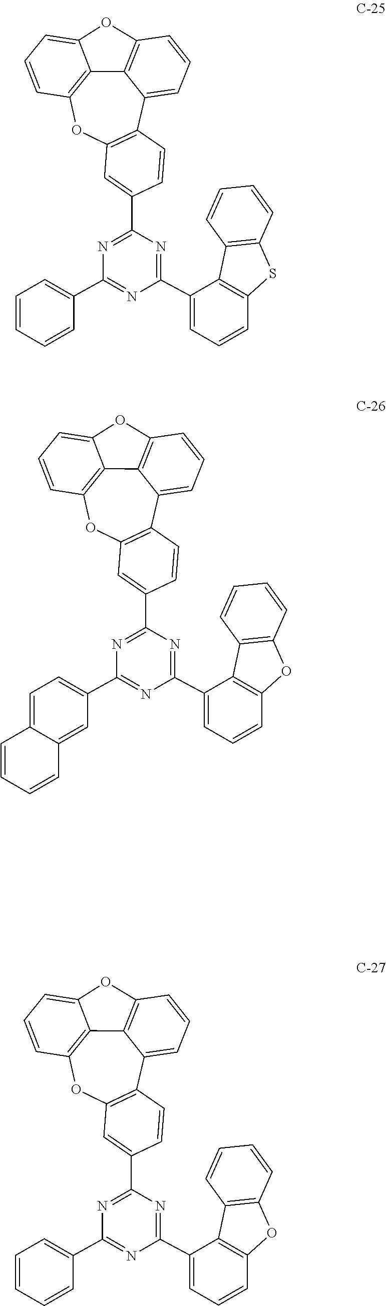 [SMILES: c1ccc(-c2nc(-c3ccc4c(c3)oc3cccc5oc6cccc4c6c35)nc(-c3cccc4sc5ccccc5c34)n2)cc1 ; c1ccc2cc(-c3nc(-c4ccc5c(c4)oc4cccc6oc7cccc5c7c46)nc(-c4cccc5oc6ccccc6c45)n3)ccc2c1 ; c1ccc(-c2nc(-c3ccc4c(c3)oc3cccc5oc6cccc4c6c35)nc(-c3cccc4oc5ccccc5c34)n2)cc1]

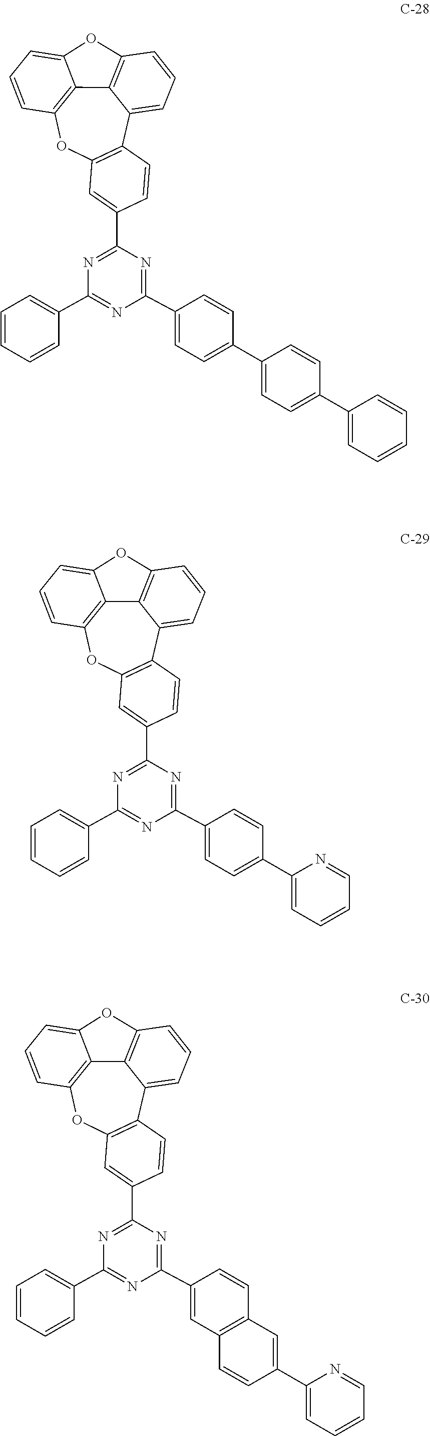 [SMILES: c1ccc(-c2ccc(-c3ccc(-c4nc(-c5ccccc5)nc(-c5ccc6c(c5)oc5cccc7oc8cccc6c8c57)n4)cc3)cc2)cc1 ; c1ccc(-c2nc(-c3ccc(-c4ccccn4)cc3)nc(-c3ccc4c(c3)oc3cccc5oc6cccc4c6c35)n2)cc1 ; c1ccc(-c2nc(-c3ccc4cc(-c5ccccn5)ccc4c3)nc(-c3ccc4c(c3)oc3cccc5oc6cccc4c6c35)n2)cc1]

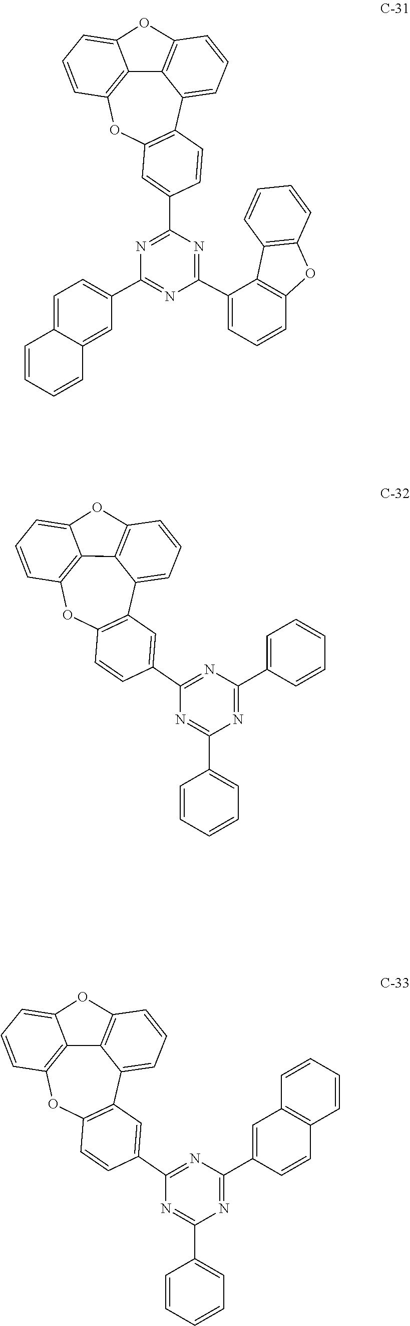 [SMILES: c1ccc2cc(-c3nc(-c4ccc5c(c4)oc4cccc6oc7cccc5c7c46)nc(-c4cccc5oc6ccccc6c45)n3)ccc2c1 ; c1ccc(-c2nc(-c3ccccc3)nc(-c3ccc4oc5cccc6oc7cccc(c4c3)c7c56)n2)cc1 ; c1ccc(-c2nc(-c3ccc4ccccc4c3)nc(-c3ccc4oc5cccc6oc7cccc(c4c3)c7c56)n2)cc1]

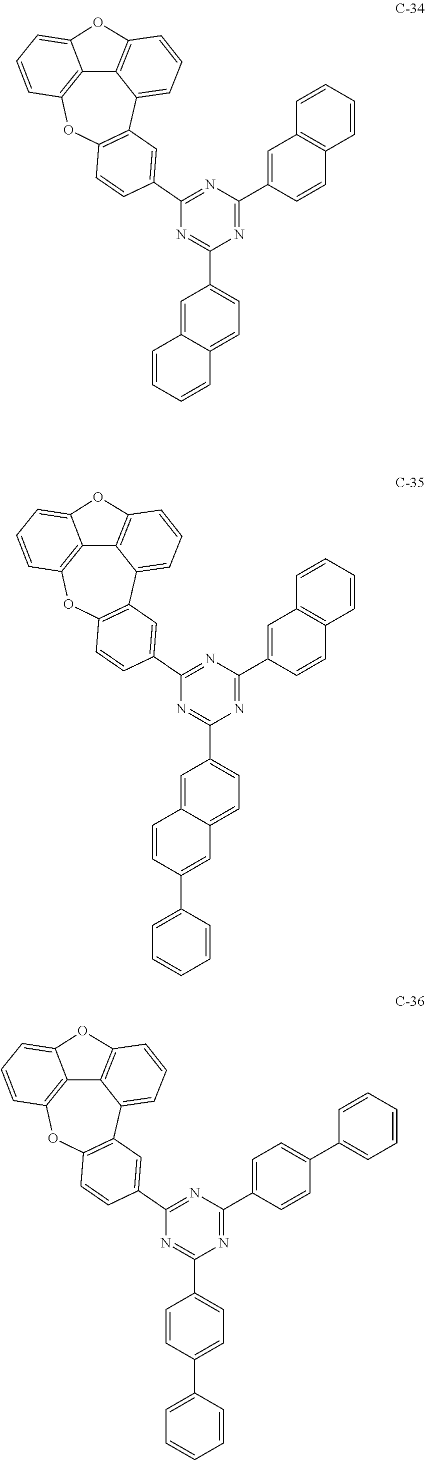 [SMILES: c1ccc2cc(-c3nc(-c4ccc5ccccc5c4)nc(-c4ccc5oc6cccc7oc8cccc(c5c4)c8c67)n3)ccc2c1 ; c1ccc(-c2ccc3cc(-c4nc(-c5ccc6ccccc6c5)nc(-c5ccc6oc7cccc8oc9cccc(c6c5)c9c78)n4)ccc3c2)cc1 ; c1ccc(-c2ccc(-c3nc(-c4ccc(-c5ccccc5)cc4)nc(-c4ccc5oc6cccc7oc8cccc(c5c4)c8c67)n3)cc2)cc1]

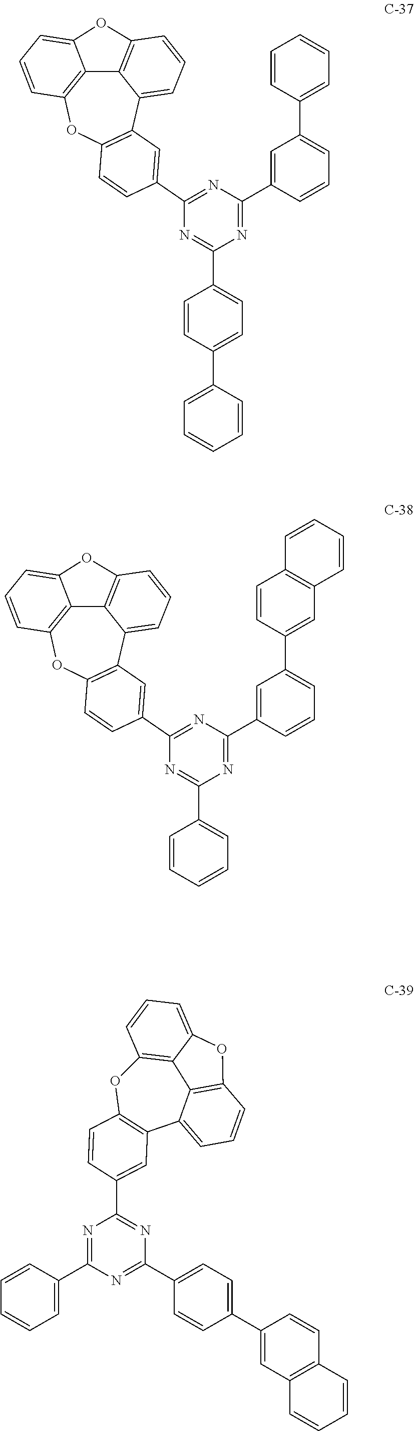 [SMILES: c1ccc(-c2ccc(-c3nc(-c4cccc(-c5ccccc5)c4)nc(-c4ccc5oc6cccc7oc8cccc(c5c4)c8c67)n3)cc2)cc1 ; c1ccc(-c2nc(-c3cccc(-c4ccc5ccccc5c4)c3)nc(-c3ccc4oc5cccc6oc7cccc(c4c3)c7c56)n2)cc1 ; c1ccc(-c2nc(-c3ccc(-c4ccc5ccccc5c4)cc3)nc(-c3ccc4oc5cccc6oc7cccc(c4c3)c7c56)n2)cc1]

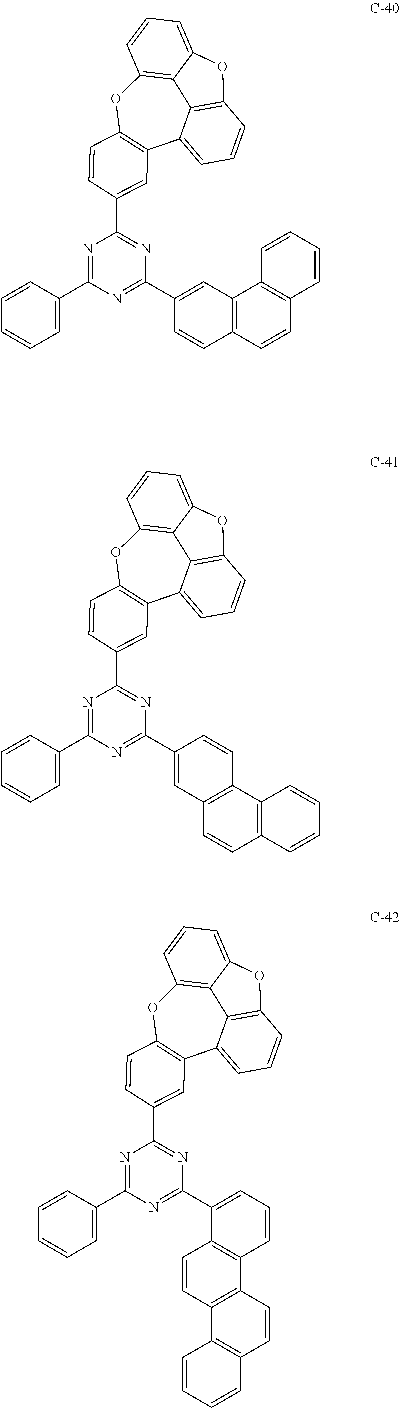 [SMILES: c1ccc(-c2nc(-c3ccc4ccc5ccccc5c4c3)nc(-c3ccc4oc5cccc6oc7cccc(c4c3)c7c56)n2)cc1 ; c1ccc(-c2nc(-c3ccc4c(ccc5ccccc54)c3)nc(-c3ccc4oc5cccc6oc7cccc(c4c3)c7c56)n2)cc1 ; c1ccc(-c2nc(-c3ccc4oc5cccc6oc7cccc(c4c3)c7c56)nc(-c3cccc4c3ccc3c5ccccc5ccc43)n2)cc1]

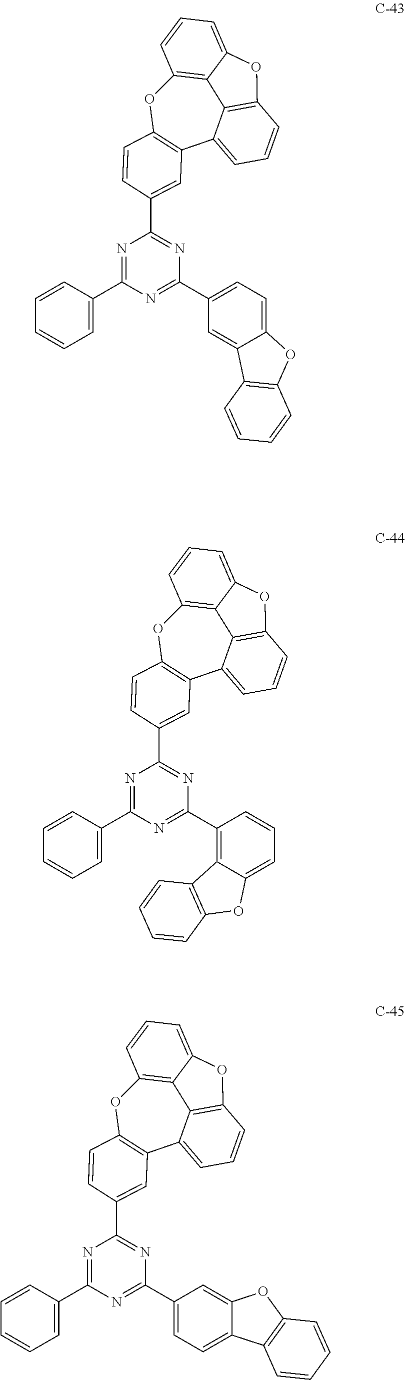 [SMILES: c1ccc(-c2nc(-c3ccc4oc5ccccc5c4c3)nc(-c3ccc4oc5cccc6oc7cccc(c4c3)c7c56)n2)cc1 ; c1ccc(-c2nc(-c3ccc4oc5cccc6oc7cccc(c4c3)c7c56)nc(-c3cccc4oc5ccccc5c34)n2)cc1 ; c1ccc(-c2nc(-c3ccc4c(c3)oc3ccccc34)nc(-c3ccc4oc5cccc6oc7cccc(c4c3)c7c56)n2)cc1]

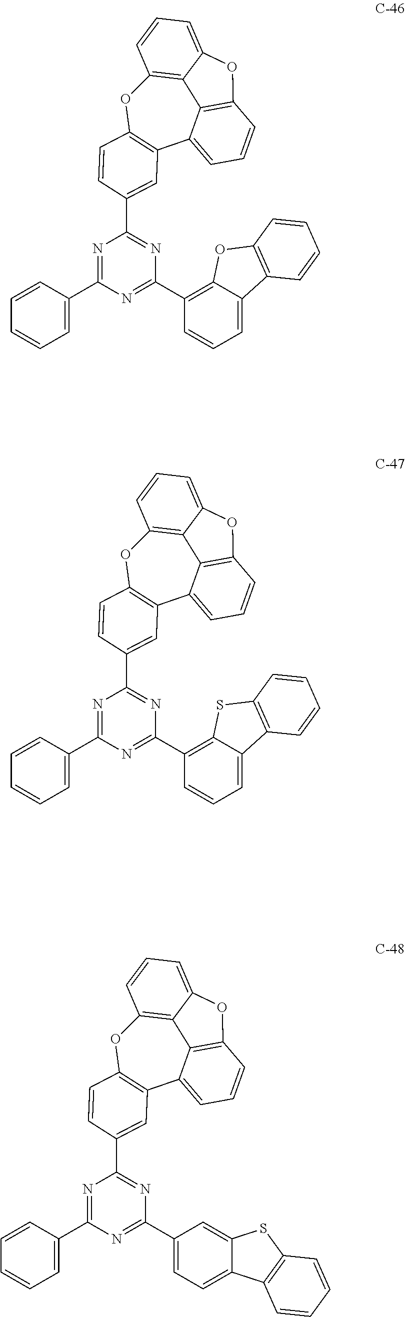 [SMILES: c1ccc(-c2nc(-c3ccc4oc5cccc6oc7cccc(c4c3)c7c56)nc(-c3cccc4c3oc3ccccc34)n2)cc1 ; c1ccc(-c2nc(-c3ccc4oc5cccc6oc7cccc(c4c3)c7c56)nc(-c3cccc4c3sc3ccccc34)n2)cc1 ; c1ccc(-c2nc(-c3ccc4c(c3)sc3ccccc34)nc(-c3ccc4oc5cccc6oc7cccc(c4c3)c7c56)n2)cc1]

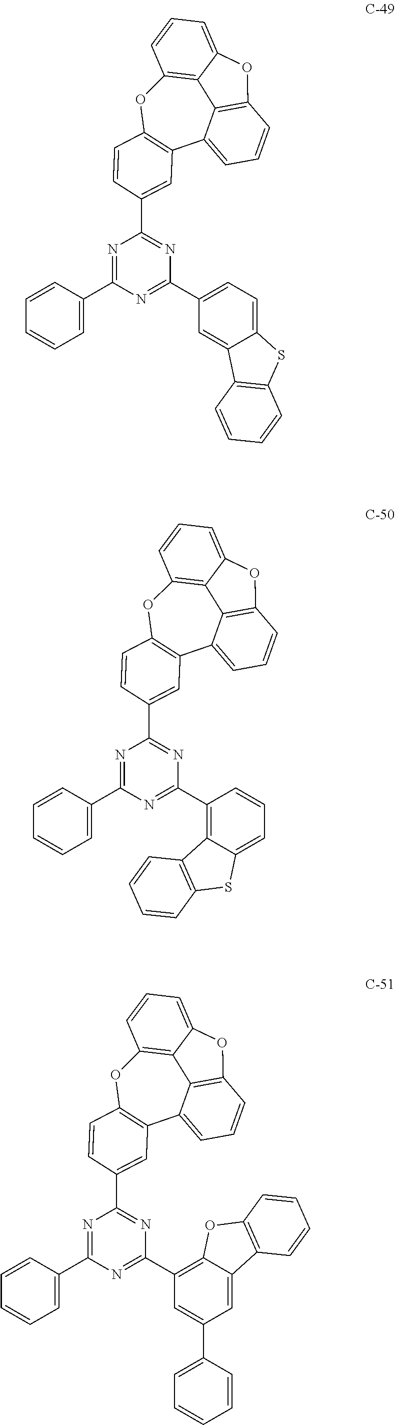 [SMILES: c1ccc(-c2nc(-c3ccc4oc5cccc6oc7cccc(c4c3)c7c56)nc(-c3ccc4sc5ccccc5c4c3)n2)cc1 ; c1ccc(-c2nc(-c3ccc4oc5cccc6oc7cccc(c4c3)c7c56)nc(-c3cccc4sc5ccccc5c34)n2)cc1 ; c1ccc(-c2cc(-c3nc(-c4ccccc4)nc(-c4ccc5oc6cccc7oc8cccc(c5c4)c8c67)n3)c3oc4ccccc4c3c2)cc1]

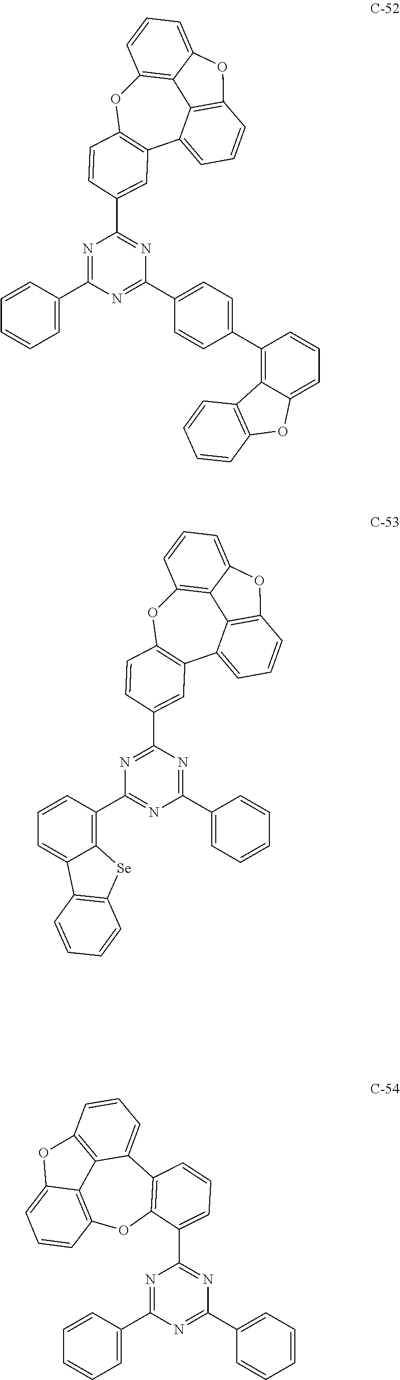 [SMILES: c1ccc(-c2nc(-c3ccc(-c4cccc5oc6ccccc6c45)cc3)nc(-c3ccc4oc5cccc6oc7cccc(c4c3)c7c56)n2)cc1 ; c1ccc(-c2nc(-c3ccc4oc5cccc6oc7cccc(c4c3)c7c56)nc(-c3cccc4c3[se]c3ccccc34)n2)cc1 ; c1ccc(-c2nc(-c3ccccc3)nc(-c3cccc4c3oc3cccc5oc6cccc4c6c53)n2)cc1]

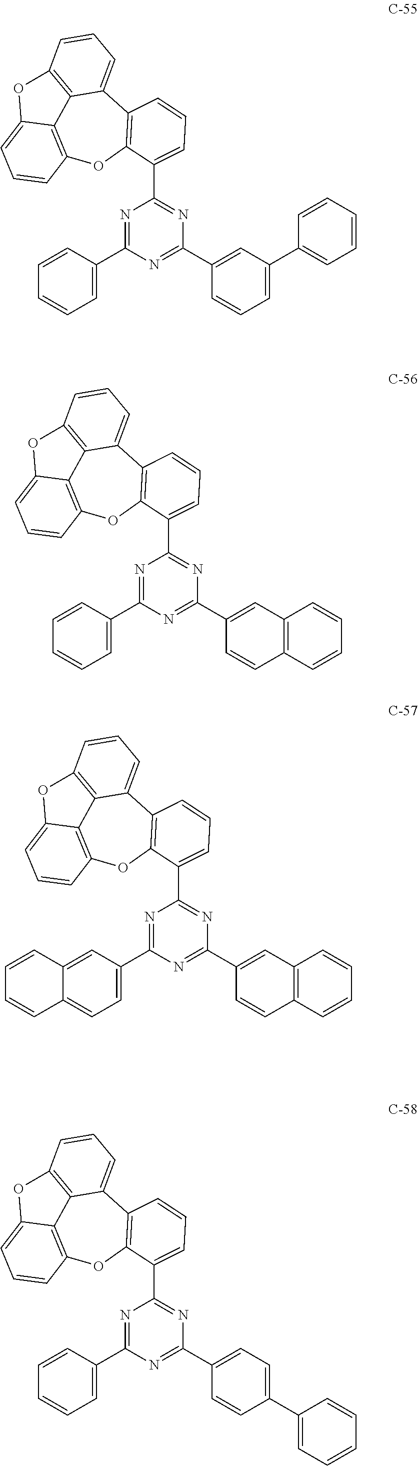 [SMILES: c1ccc(-c2cccc(-c3nc(-c4ccccc4)nc(-c4cccc5c4oc4cccc6oc7cccc5c7c64)n3)c2)cc1 ; c1ccc(-c2nc(-c3ccc4ccccc4c3)nc(-c3cccc4c3oc3cccc5oc6cccc4c6c53)n2)cc1 ; c1ccc2cc(-c3nc(-c4ccc5ccccc5c4)nc(-c4cccc5c4oc4cccc6oc7cccc5c7c64)n3)ccc2c1 ; c1ccc(-c2ccc(-c3nc(-c4ccccc4)nc(-c4cccc5c4oc4cccc6oc7cccc5c7c64)n3)cc2)cc1]

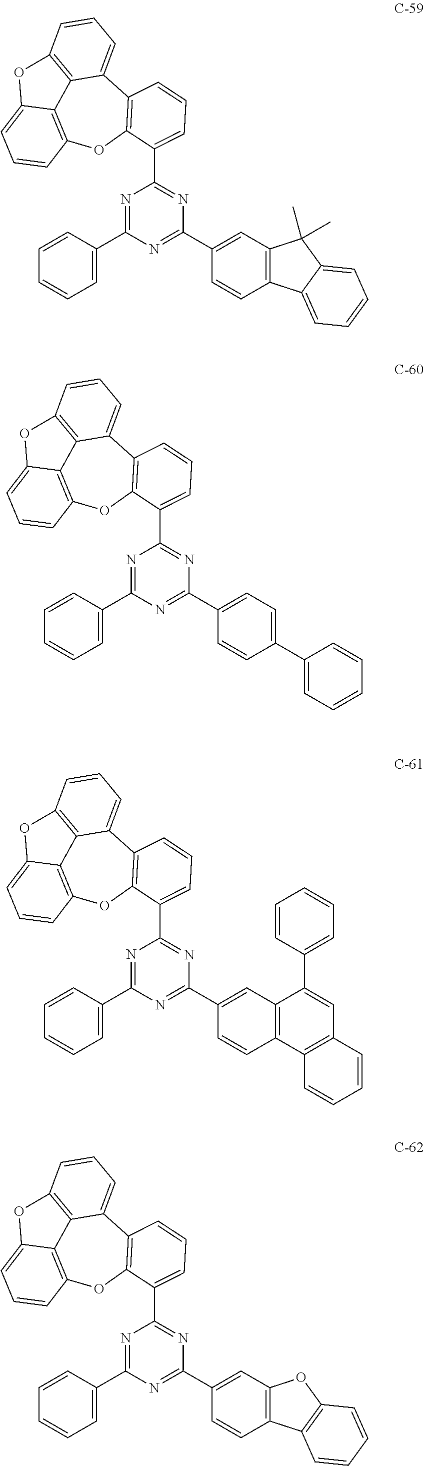 [SMILES: CC1(C)c2ccccc2-c2ccc(-c3nc(-c4ccccc4)nc(-c4cccc5c4oc4cccc6oc7cccc5c7c64)n3)cc21 ; c1ccc(-c2ccc(-c3nc(-c4ccccc4)nc(-c4cccc5c4oc4cccc6oc7cccc5c7c64)n3)cc2)cc1 ; c1ccc(-c2nc(-c3ccc4c(c3)c(-c3ccccc3)cc3ccccc34)nc(-c3cccc4c3oc3cccc5oc6cccc4c6c53)n2)cc1 ; c1ccc(-c2nc(-c3ccc4c(c3)oc3ccccc34)nc(-c3cccc4c3oc3cccc5oc6cccc4c6c53)n2)cc1]

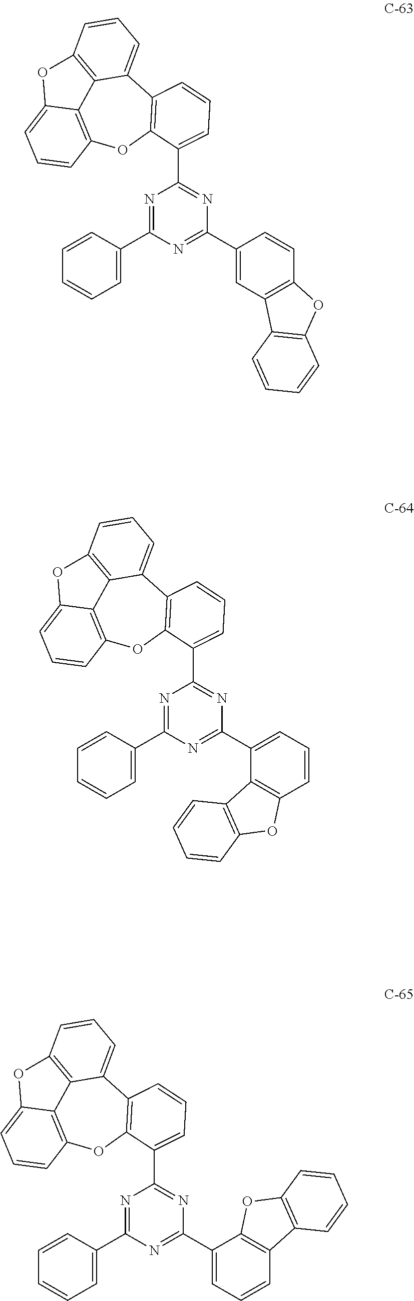 [SMILES: c1ccc(-c2nc(-c3ccc4oc5ccccc5c4c3)nc(-c3cccc4c3oc3cccc5oc6cccc4c6c53)n2)cc1 ; c1ccc(-c2nc(-c3cccc4c3oc3cccc5oc6cccc4c6c53)nc(-c3cccc4oc5ccccc5c34)n2)cc1 ; c1ccc(-c2nc(-c3cccc4c3oc3ccccc34)nc(-c3cccc4c3oc3cccc5oc6cccc4c6c53)n2)cc1]

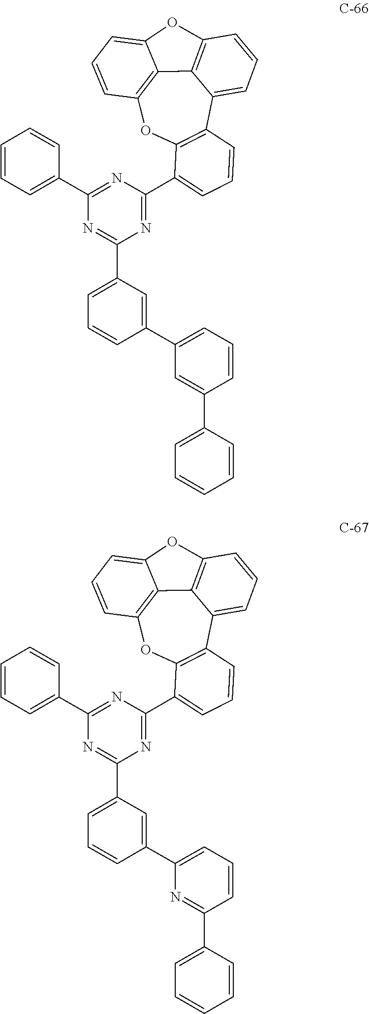 [SMILES: c1ccc(-c2cccc(-c3cccc(-c4nc(-c5ccccc5)nc(-c5cccc6c5oc5cccc7oc8cccc6c8c75)n4)c3)c2)cc1 ; c1ccc(-c2cccc(-c3cccc(-c4nc(-c5ccccc5)nc(-c5cccc6c5oc5cccc7oc8cccc6c8c75)n4)c3)n2)cc1]

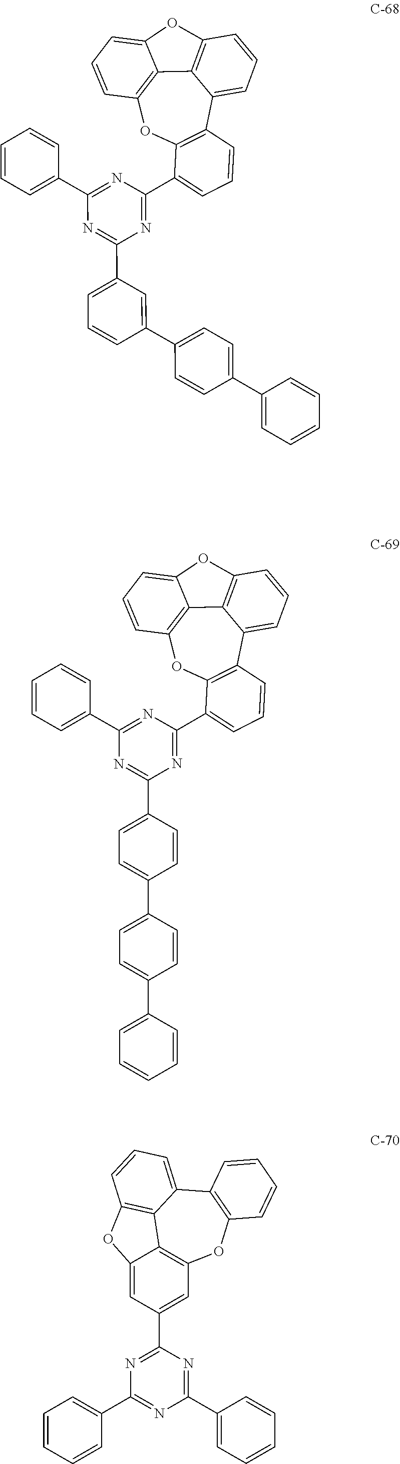 [SMILES: c1ccc(-c2ccc(-c3cccc(-c4nc(-c5ccccc5)nc(-c5cccc6c5oc5cccc7oc8cccc6c8c75)n4)c3)cc2)cc1 ; c1ccc(-c2ccc(-c3ccc(-c4nc(-c5ccccc5)nc(-c5cccc6c5oc5cccc7oc8cccc6c8c75)n4)cc3)cc2)cc1 ; c1ccc(-c2nc(-c3ccccc3)nc(-c3cc4oc5ccccc5c5cccc6oc(c3)c4c65)n2)cc1]

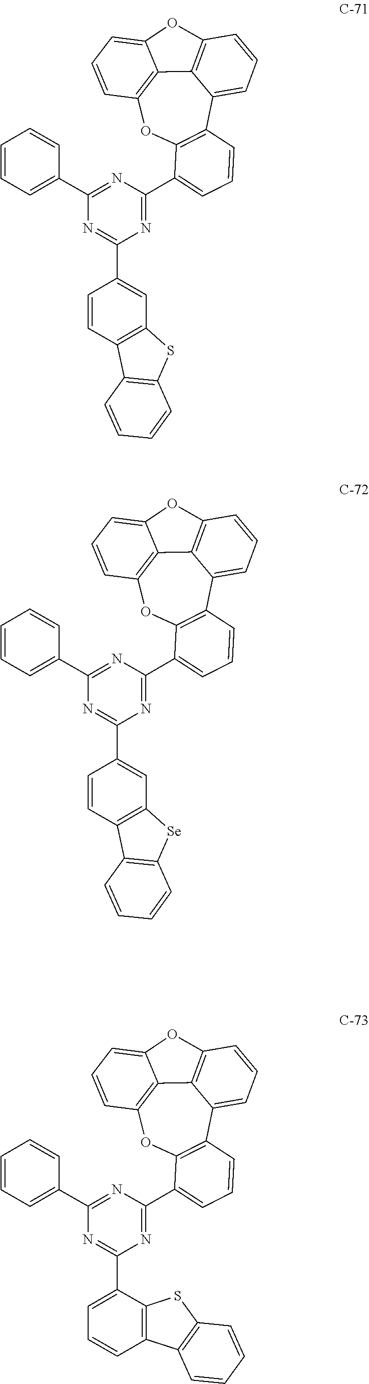 [SMILES: c1ccc(-c2nc(-c3ccc4c(c3)sc3ccccc34)nc(-c3cccc4c3oc3cccc5oc6cccc4c6c53)n2)cc1 ; c1ccc(-c2nc(-c3ccc4c(c3)[se]c3ccccc34)nc(-c3cccc4c3oc3cccc5oc6cccc4c6c53)n2)cc1 ; c1ccc(-c2nc(-c3cccc4c3oc3cccc5oc6cccc4c6c53)nc(-c3cccc4c3sc3ccccc34)n2)cc1]

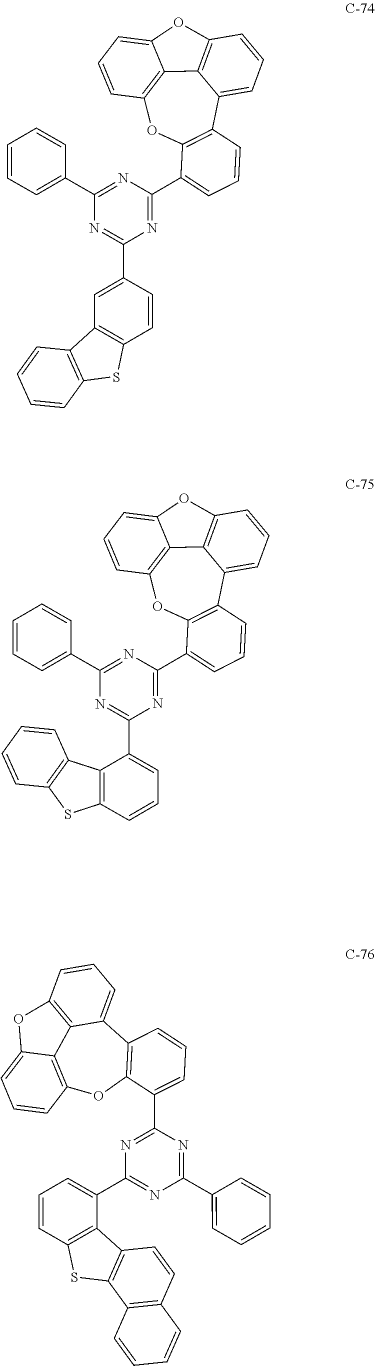 [SMILES: c1ccc(-c2nc(-c3ccc4sc5ccccc5c4c3)nc(-c3cccc4c3oc3cccc5oc6cccc4c6c53)n2)cc1 ; c1ccc(-c2nc(-c3cccc4c3oc3cccc5oc6cccc4c6c53)nc(-c3cccc4sc5ccccc5c34)n2)cc1 ; c1ccc(-c2nc(-c3cccc4c3oc3cccc5oc6cccc4c6c53)nc(-c3cccc4sc5c6ccccc6ccc5c34)n2)cc1]

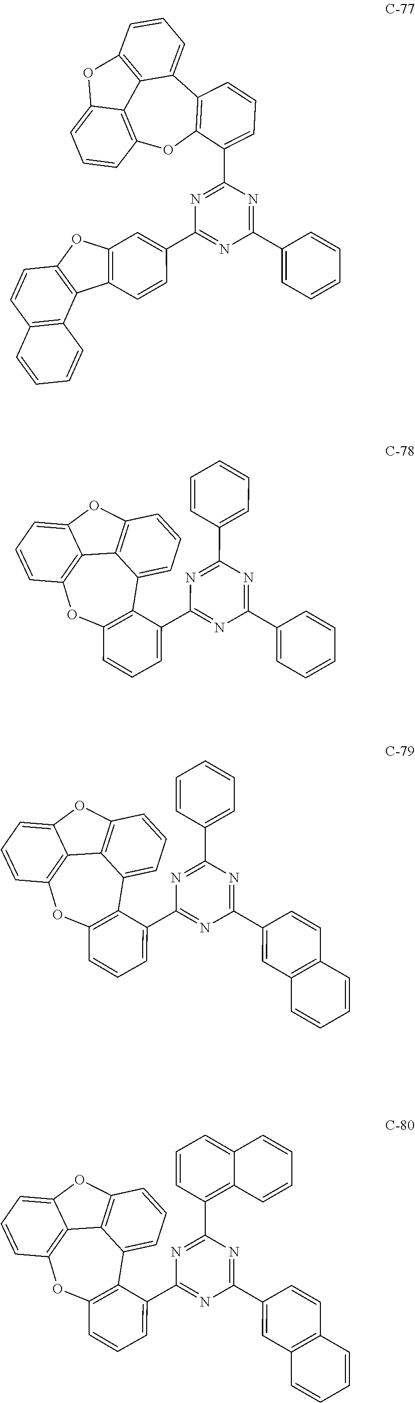 [SMILES: c1ccc(-c2nc(-c3ccc4c(c3)oc3ccc5ccccc5c34)nc(-c3cccc4c3oc3cccc5oc6cccc4c6c53)n2)cc1 ; c1ccc(-c2nc(-c3ccccc3)nc(-c3cccc4oc5cccc6oc7cccc(c34)c7c56)n2)cc1 ; c1ccc(-c2nc(-c3ccc4ccccc4c3)nc(-c3cccc4oc5cccc6oc7cccc(c34)c7c56)n2)cc1 ; c1ccc2cc(-c3nc(-c4cccc5ccccc45)nc(-c4cccc5oc6cccc7oc8cccc(c45)c8c67)n3)ccc2c1]

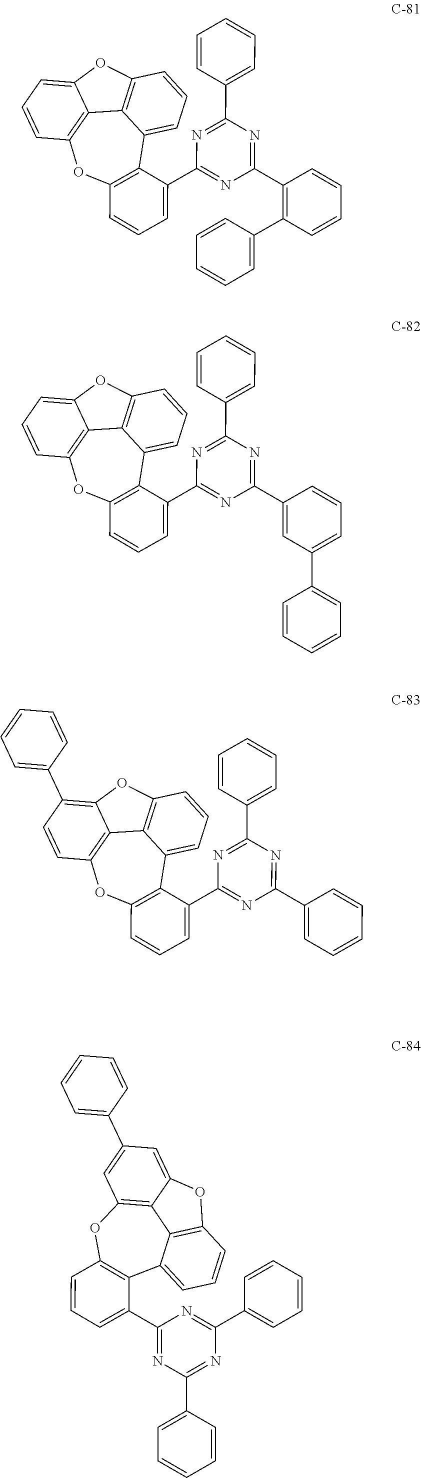 [SMILES: c1ccc(-c2nc(-c3ccccc3-c3ccccc3)nc(-c3cccc4oc5cccc6oc7cccc(c34)c7c56)n2)cc1 ; c1ccc(-c2cccc(-c3nc(-c4ccccc4)nc(-c4cccc5oc6cccc7oc8cccc(c45)c8c67)n3)c2)cc1 ; c1ccc(-c2nc(-c3ccccc3)nc(-c3cccc4oc5ccc(-c6ccccc6)c6oc7cccc(c34)c7c56)n2)cc1 ; c1ccc(-c2cc3oc4cccc(-c5nc(-c6ccccc6)nc(-c6ccccc6)n5)c4c4cccc5oc(c2)c3c54)cc1]

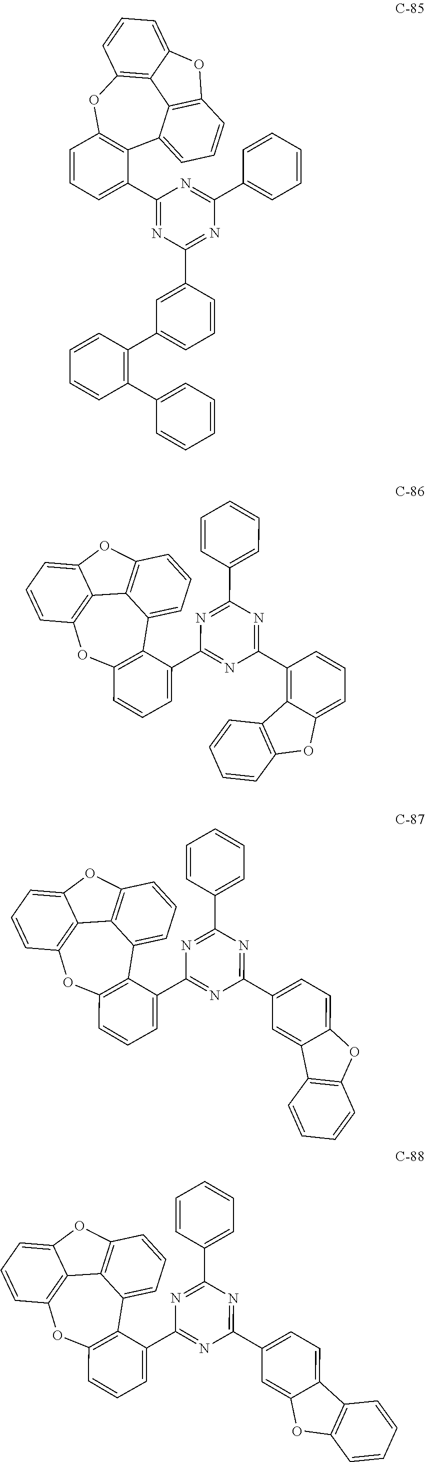 [SMILES: c1ccc(-c2nc(-c3cccc(-c4ccccc4-c4ccccc4)c3)nc(-c3cccc4oc5cccc6oc7cccc(c34)c7c56)n2)cc1 ; c1ccc(-c2nc(-c3cccc4oc5ccccc5c34)nc(-c3cccc4oc5cccc6oc7cccc(c34)c7c56)n2)cc1 ; c1ccc(-c2nc(-c3ccc4oc5ccccc5c4c3)nc(-c3cccc4oc5cccc6oc7cccc(c34)c7c56)n2)cc1 ; c1ccc(-c2nc(-c3ccc4c(c3)oc3ccccc34)nc(-c3cccc4oc5cccc6oc7cccc(c34)c7c56)n2)cc1]

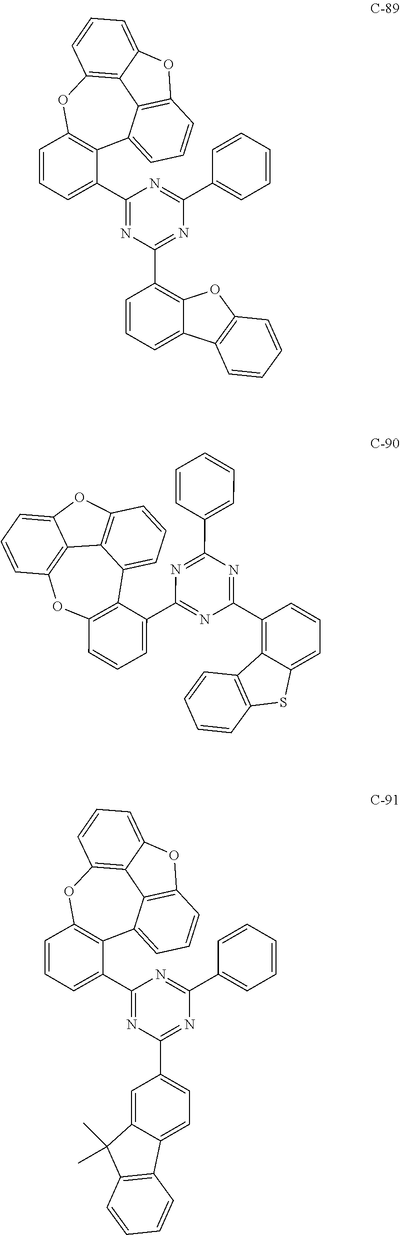 [SMILES: c1ccc(-c2nc(-c3cccc4c3oc3ccccc34)nc(-c3cccc4oc5cccc6oc7cccc(c34)c7c56)n2)cc1 ; c1ccc(-c2nc(-c3cccc4oc5cccc6oc7cccc(c34)c7c56)nc(-c3cccc4sc5ccccc5c34)n2)cc1 ; CC1(C)c2ccccc2-c2ccc(-c3nc(-c4ccccc4)nc(-c4cccc5oc6cccc7oc8cccc(c45)c8c67)n3)cc21]

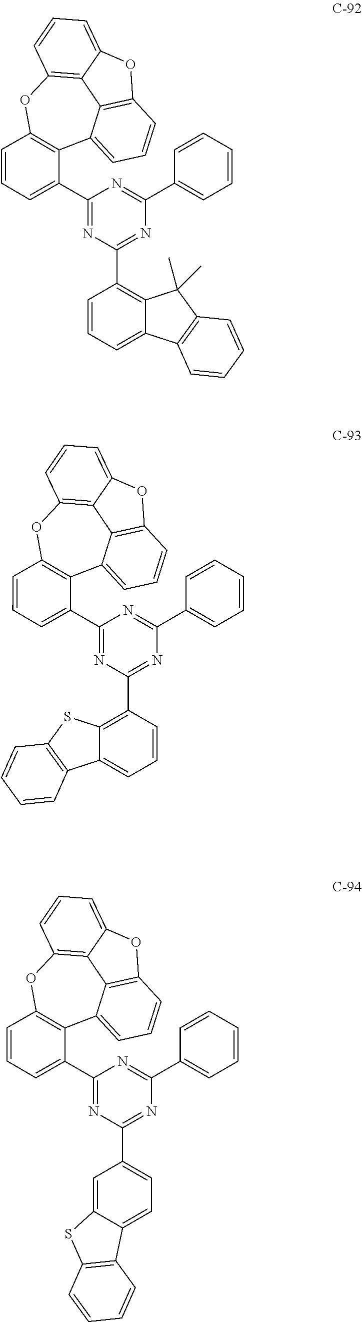 [SMILES: CC1(C)c2ccccc2-c2cccc(-c3nc(-c4ccccc4)nc(-c4cccc5oc6cccc7oc8cccc(c45)c8c67)n3)c21 ; c1ccc(-c2nc(-c3cccc4c3sc3ccccc34)nc(-c3cccc4oc5cccc6oc7cccc(c34)c7c56)n2)cc1 ; c1ccc(-c2nc(-c3ccc4c(c3)sc3ccccc34)nc(-c3cccc4oc5cccc6oc7cccc(c34)c7c56)n2)cc1]

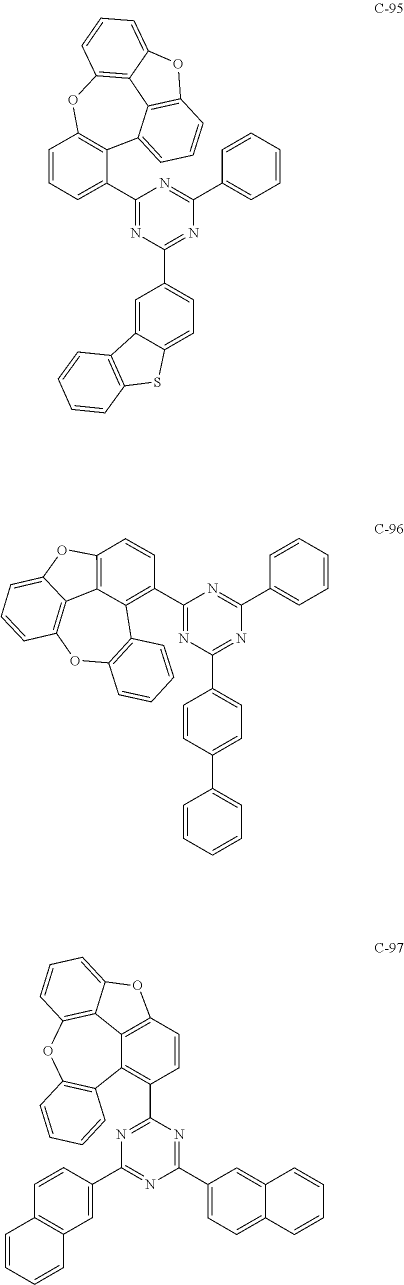 [SMILES: c1ccc(-c2nc(-c3ccc4sc5ccccc5c4c3)nc(-c3cccc4oc5cccc6oc7cccc(c34)c7c56)n2)cc1 ; c1ccc(-c2ccc(-c3nc(-c4ccccc4)nc(-c4ccc5oc6cccc7oc8ccccc8c4c5c76)n3)cc2)cc1 ; c1ccc2cc(-c3nc(-c4ccc5ccccc5c4)nc(-c4ccc5oc6cccc7oc8ccccc8c4c5c76)n3)ccc2c1]

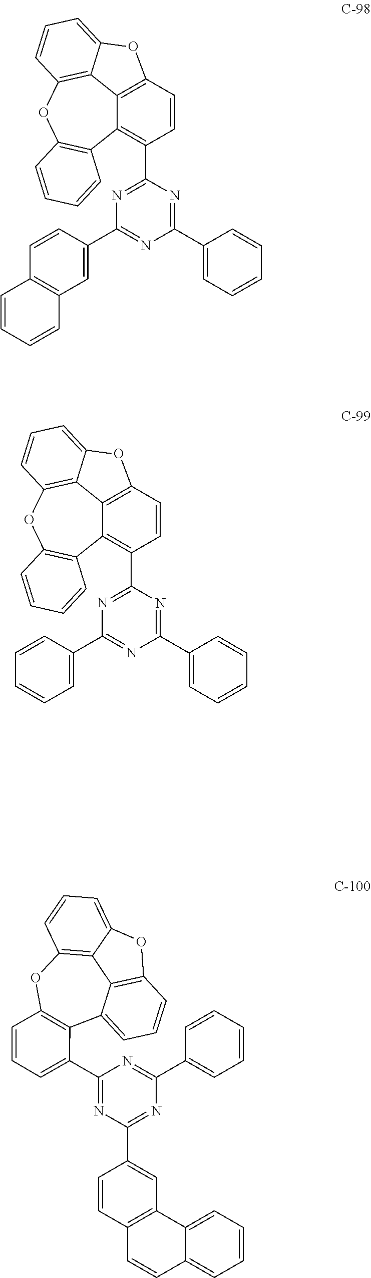 [SMILES: c1ccc(-c2nc(-c3ccc4ccccc4c3)nc(-c3ccc4oc5cccc6oc7ccccc7c3c4c65)n2)cc1 ; c1ccc(-c2nc(-c3ccccc3)nc(-c3ccc4oc5cccc6oc7ccccc7c3c4c65)n2)cc1 ; c1ccc(-c2nc(-c3ccc4ccc5ccccc5c4c3)nc(-c3cccc4oc5cccc6oc7cccc(c34)c7c56)n2)cc1]

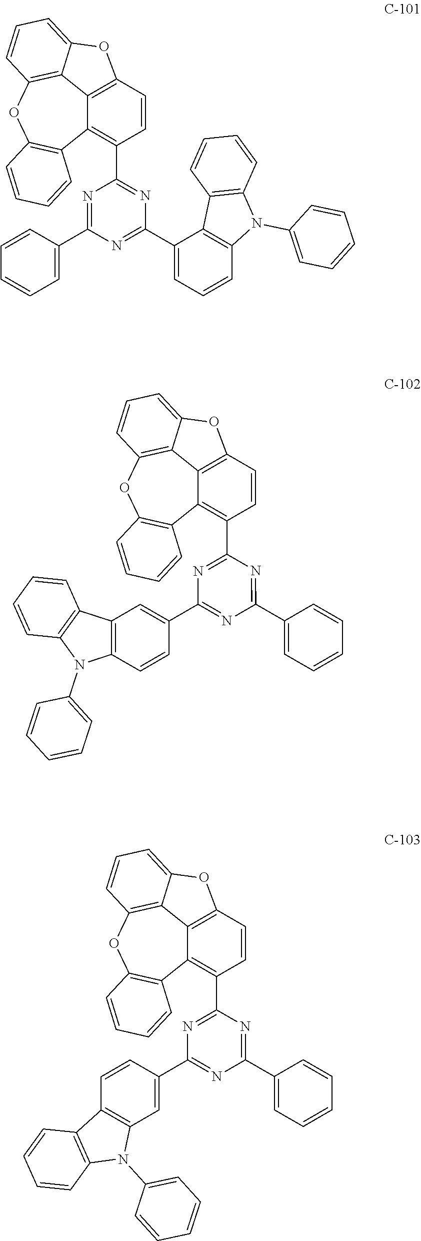 [SMILES: c1ccc(-c2nc(-c3ccc4oc5cccc6oc7ccccc7c3c4c65)nc(-c3cccc4c3c3ccccc3n4-c3ccccc3)n2)cc1 ; c1ccc(-c2nc(-c3ccc4c(c3)c3ccccc3n4-c3ccccc3)nc(-c3ccc4oc5cccc6oc7ccccc7c3c4c65)n2)cc1 ; c1ccc(-c2nc(-c3ccc4c5ccccc5n(-c5ccccc5)c4c3)nc(-c3ccc4oc5cccc6oc7ccccc7c3c4c65)n2)cc1]

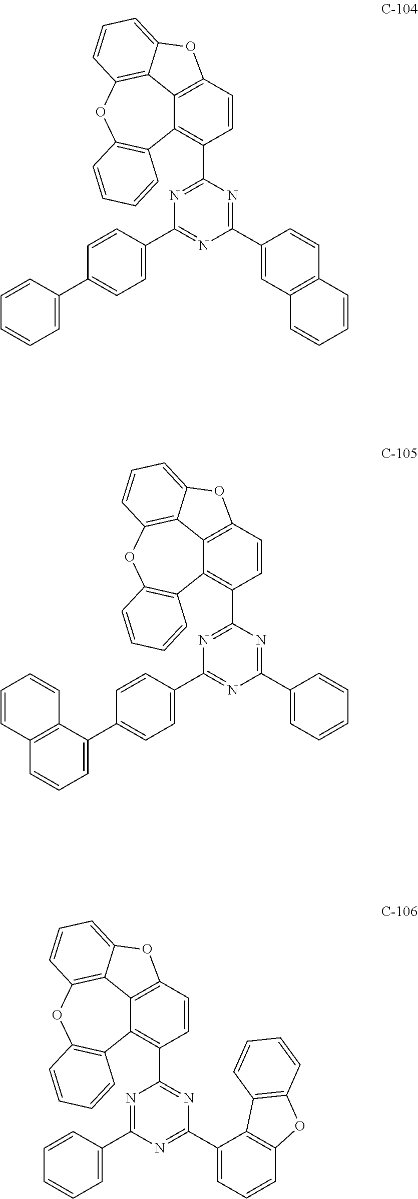 [SMILES: c1ccc(-c2ccc(-c3nc(-c4ccc5ccccc5c4)nc(-c4ccc5oc6cccc7oc8ccccc8c4c5c76)n3)cc2)cc1 ; c1ccc(-c2nc(-c3ccc(-c4cccc5ccccc45)cc3)nc(-c3ccc4oc5cccc6oc7ccccc7c3c4c65)n2)cc1 ; c1ccc(-c2nc(-c3cccc4oc5ccccc5c34)nc(-c3ccc4oc5cccc6oc7ccccc7c3c4c65)n2)cc1]

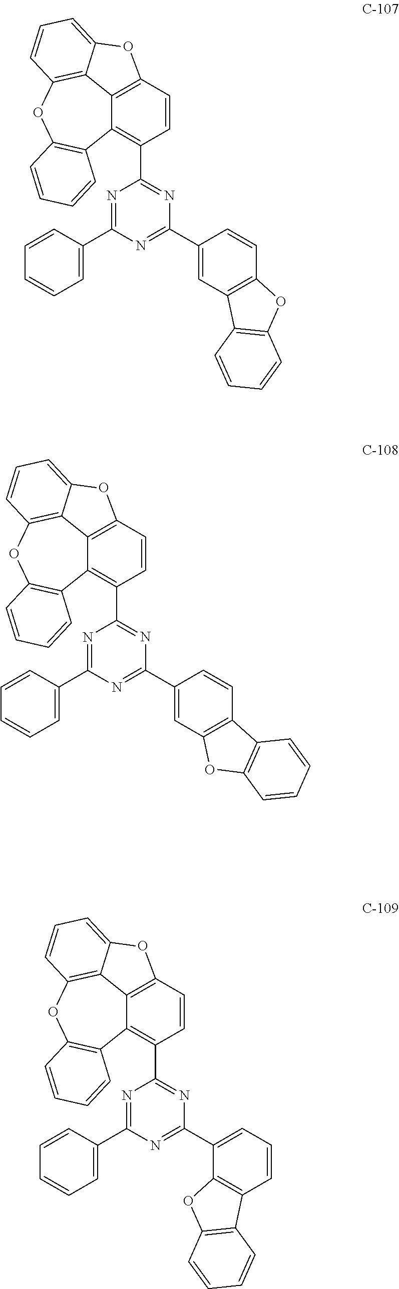 [SMILES: c1ccc(-c2nc(-c3ccc4oc5ccccc5c4c3)nc(-c3ccc4oc5cccc6oc7ccccc7c3c4c65)n2)cc1 ; c1ccc(-c2nc(-c3ccc4c(c3)oc3ccccc34)nc(-c3ccc4oc5cccc6oc7ccccc7c3c4c65)n2)cc1 ; c1ccc(-c2nc(-c3cccc4c3oc3ccccc34)nc(-c3ccc4oc5cccc6oc7ccccc7c3c4c65)n2)cc1]

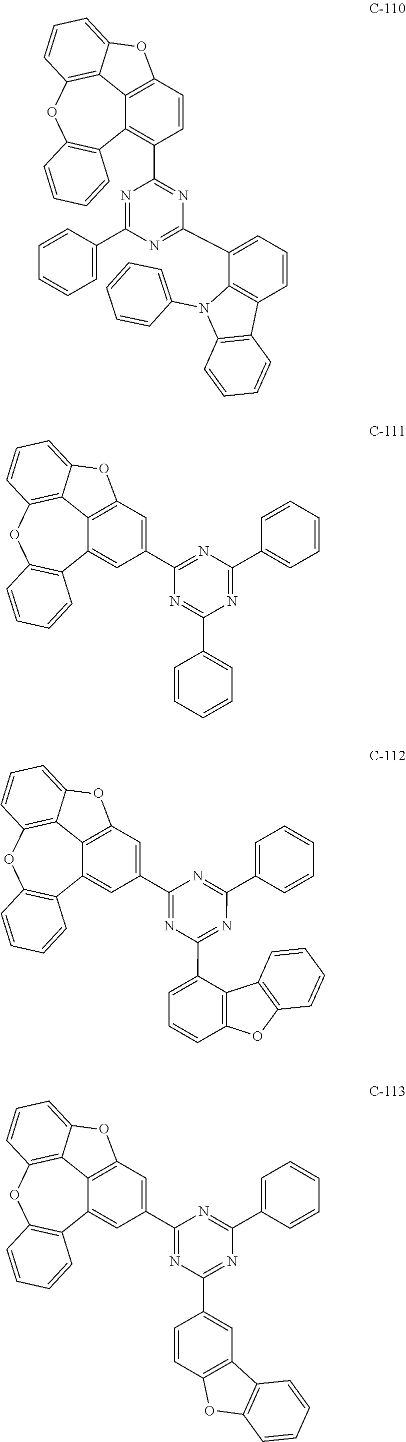 [SMILES: c1ccc(-c2nc(-c3ccc4oc5cccc6oc7ccccc7c3c4c65)nc(-c3cccc4c5ccccc5n(-c5ccccc5)c34)n2)cc1 ; c1ccc(-c2nc(-c3ccccc3)nc(-c3cc4oc5cccc6oc7ccccc7c(c3)c4c65)n2)cc1 ; c1ccc(-c2nc(-c3cc4oc5cccc6oc7ccccc7c(c3)c4c65)nc(-c3cccc4oc5ccccc5c34)n2)cc1 ; c1ccc(-c2nc(-c3ccc4oc5ccccc5c4c3)nc(-c3cc4oc5cccc6oc7ccccc7c(c3)c4c65)n2)cc1]

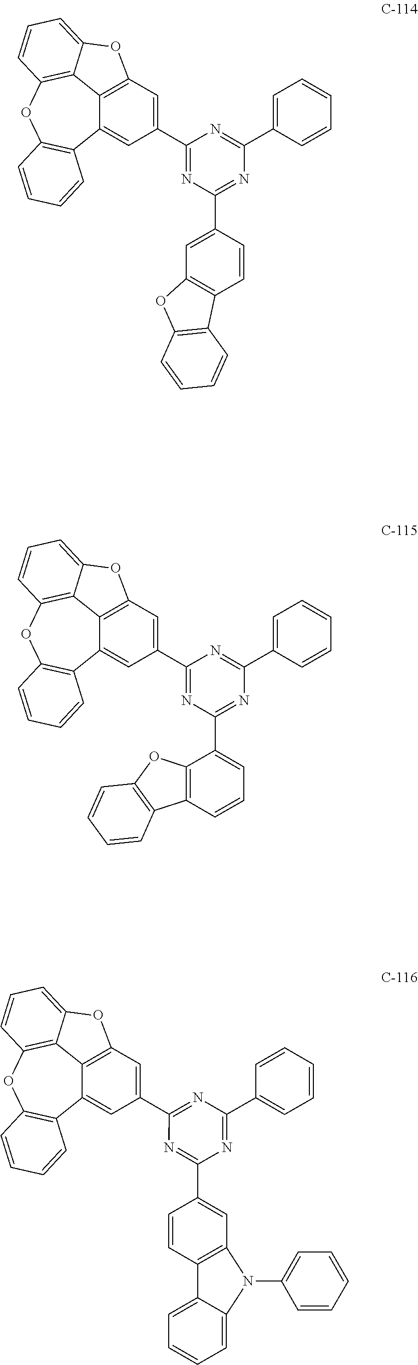 [SMILES: c1ccc(-c2nc(-c3ccc4c(c3)oc3ccccc34)nc(-c3cc4oc5cccc6oc7ccccc7c(c3)c4c65)n2)cc1 ; c1ccc(-c2nc(-c3cc4oc5cccc6oc7ccccc7c(c3)c4c65)nc(-c3cccc4c3oc3ccccc34)n2)cc1 ; c1ccc(-c2nc(-c3cc4oc5cccc6oc7ccccc7c(c3)c4c65)nc(-c3ccc4c5ccccc5n(-c5ccccc5)c4c3)n2)cc1]

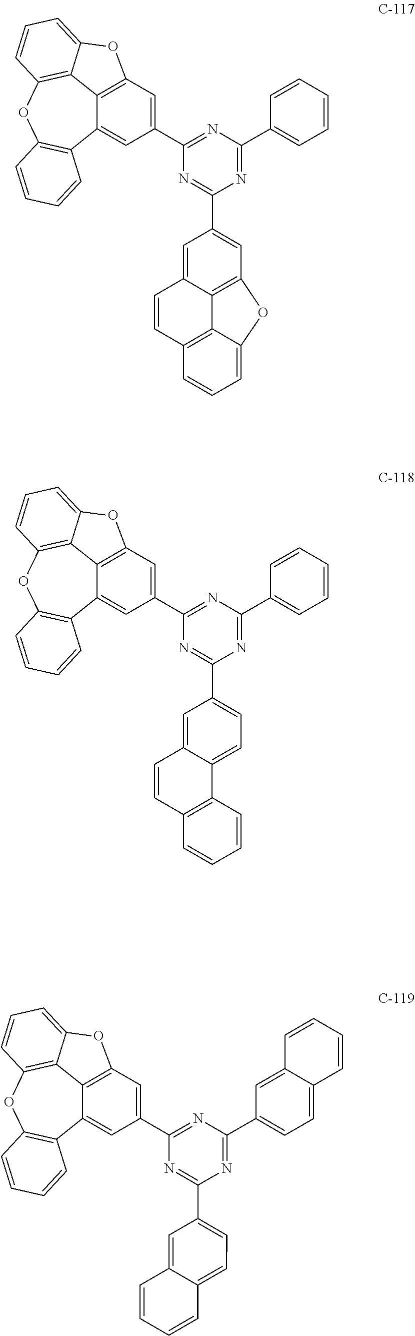 [SMILES: c1ccc(-c2nc(-c3cc4ccc5cccc6oc(c3)c4c56)nc(-c3cc4oc5cccc6oc7ccccc7c(c3)c4c65)n2)cc1 ; c1ccc(-c2nc(-c3ccc4c(ccc5ccccc54)c3)nc(-c3cc4oc5cccc6oc7ccccc7c(c3)c4c65)n2)cc1 ; c1ccc2cc(-c3nc(-c4ccc5ccccc5c4)nc(-c4cc5oc6cccc7oc8ccccc8c(c4)c5c76)n3)ccc2c1]

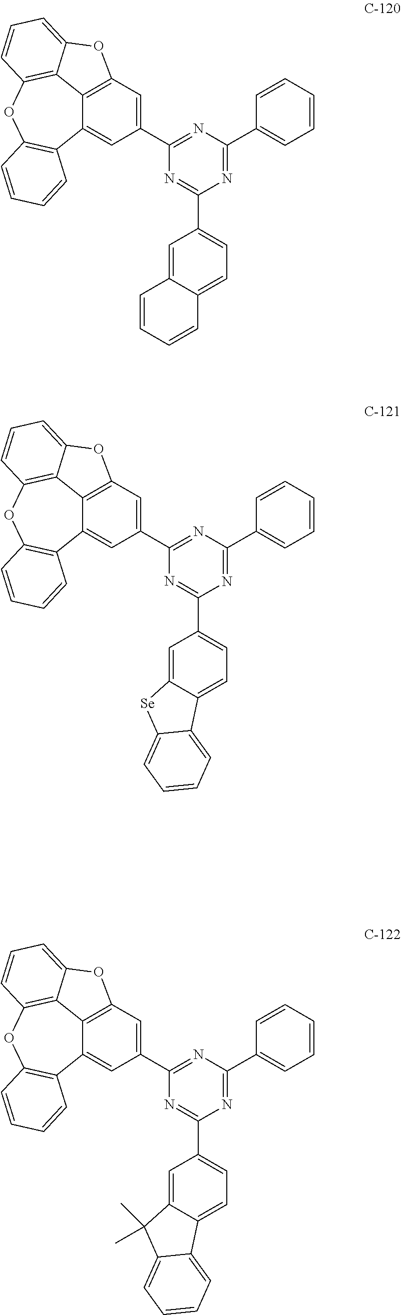 [SMILES: c1ccc(-c2nc(-c3ccc4ccccc4c3)nc(-c3cc4oc5cccc6oc7ccccc7c(c3)c4c65)n2)cc1 ; c1ccc(-c2nc(-c3ccc4c(c3)[se]c3ccccc34)nc(-c3cc4oc5cccc6oc7ccccc7c(c3)c4c65)n2)cc1 ; CC1(C)c2ccccc2-c2ccc(-c3nc(-c4ccccc4)nc(-c4cc5oc6cccc7oc8ccccc8c(c4)c5c76)n3)cc21]

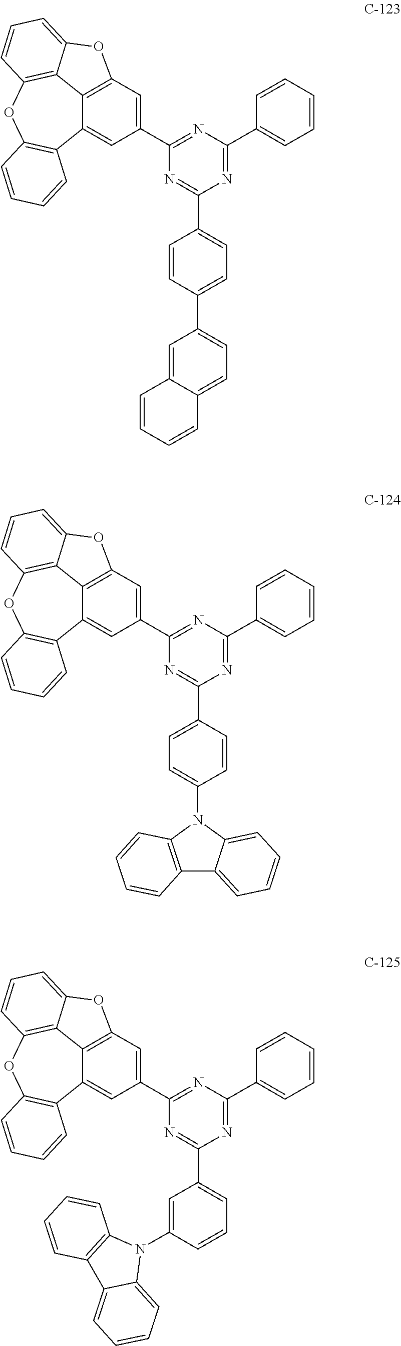 [SMILES: c1ccc(-c2nc(-c3ccc(-c4ccc5ccccc5c4)cc3)nc(-c3cc4oc5cccc6oc7ccccc7c(c3)c4c65)n2)cc1 ; c1ccc(-c2nc(-c3ccc(-n4c5ccccc5c5ccccc54)cc3)nc(-c3cc4oc5cccc6oc7ccccc7c(c3)c4c65)n2)cc1 ; c1ccc(-c2nc(-c3cccc(-n4c5ccccc5c5ccccc54)c3)nc(-c3cc4oc5cccc6oc7ccccc7c(c3)c4c65)n2)cc1]

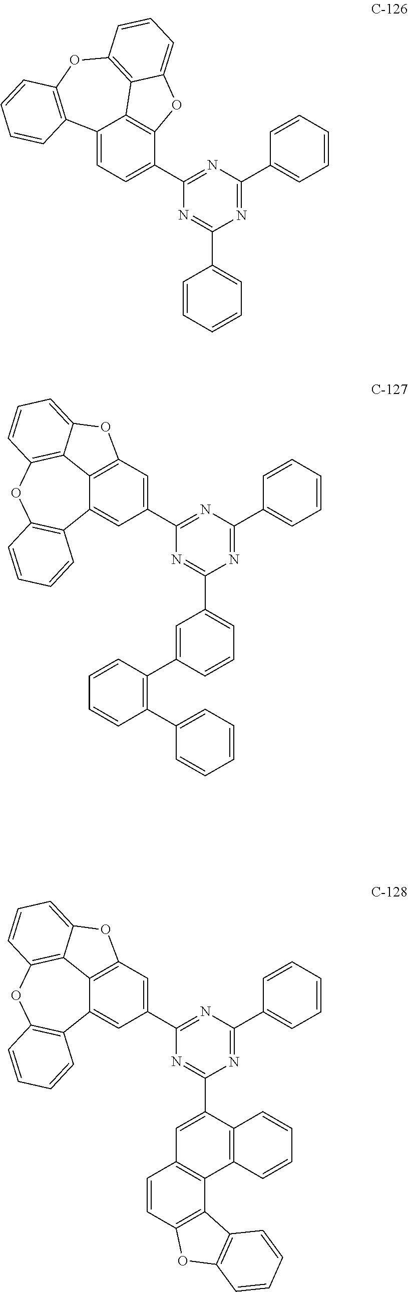 [SMILES: c1ccc(-c2nc(-c3ccccc3)nc(-c3ccc4c5ccccc5oc5cccc6oc3c4c56)n2)cc1 ; c1ccc(-c2nc(-c3cccc(-c4ccccc4-c4ccccc4)c3)nc(-c3cc4oc5cccc6oc7ccccc7c(c3)c4c65)n2)cc1 ; c1ccc(-c2nc(-c3cc4oc5cccc6oc7ccccc7c(c3)c4c65)nc(-c3cc4ccc5oc6ccccc6c5c4c4ccccc34)n2)cc1]

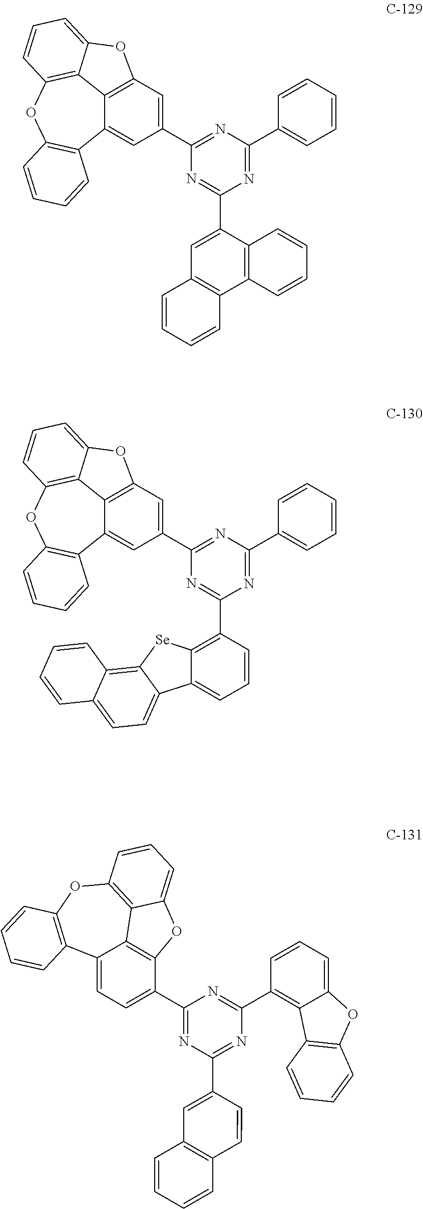 [SMILES: c1ccc(-c2nc(-c3cc4oc5cccc6oc7ccccc7c(c3)c4c65)nc(-c3cc4ccccc4c4ccccc34)n2)cc1 ; c1ccc(-c2nc(-c3cc4oc5cccc6oc7ccccc7c(c3)c4c65)nc(-c3cccc4c3[se]c3c5ccccc5ccc43)n2)cc1 ; c1ccc2cc(-c3nc(-c4ccc5c6ccccc6oc6cccc7oc4c5c67)nc(-c4cccc5oc6ccccc6c45)n3)ccc2c1]

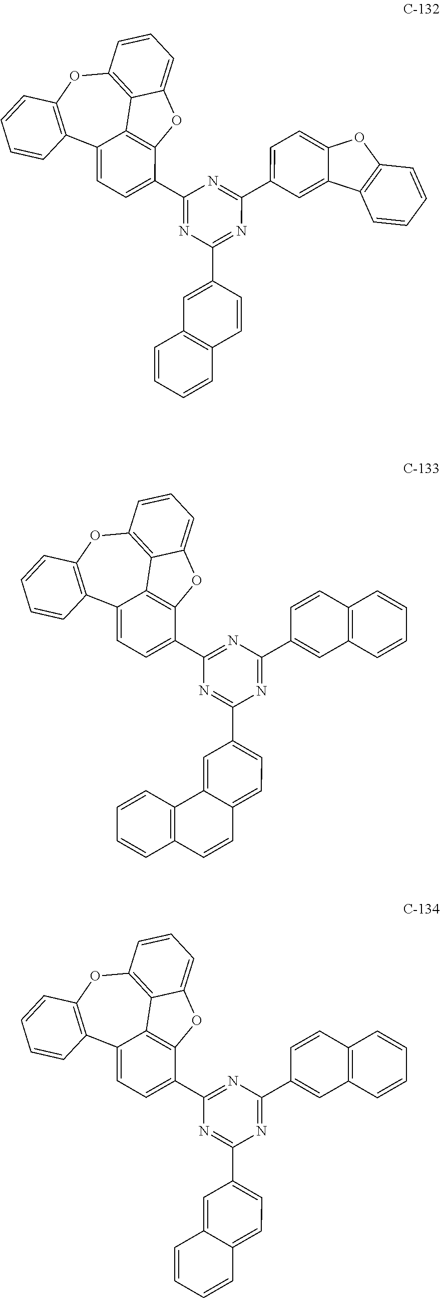 [SMILES: c1ccc2cc(-c3nc(-c4ccc5oc6ccccc6c5c4)nc(-c4ccc5c6ccccc6oc6cccc7oc4c5c67)n3)ccc2c1 ; c1ccc2cc(-c3nc(-c4ccc5ccc6ccccc6c5c4)nc(-c4ccc5c6ccccc6oc6cccc7oc4c5c67)n3)ccc2c1 ; c1ccc2cc(-c3nc(-c4ccc5ccccc5c4)nc(-c4ccc5c6ccccc6oc6cccc7oc4c5c67)n3)ccc2c1]

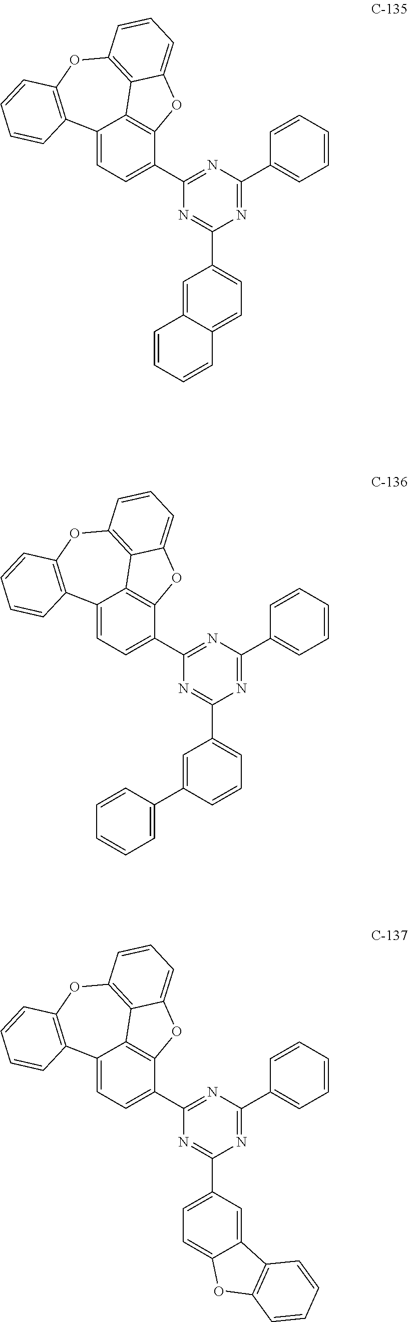 [SMILES: c1ccc(-c2nc(-c3ccc4ccccc4c3)nc(-c3ccc4c5ccccc5oc5cccc6oc3c4c56)n2)cc1 ; c1ccc(-c2cccc(-c3nc(-c4ccccc4)nc(-c4ccc5c6ccccc6oc6cccc7oc4c5c67)n3)c2)cc1 ; c1ccc(-c2nc(-c3ccc4oc5ccccc5c4c3)nc(-c3ccc4c5ccccc5oc5cccc6oc3c4c56)n2)cc1]

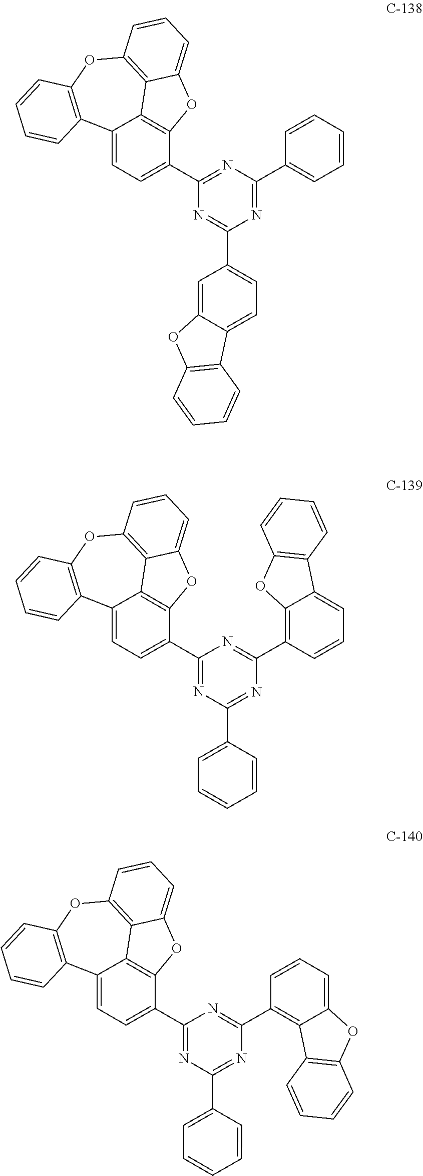 [SMILES: c1ccc(-c2nc(-c3ccc4c(c3)oc3ccccc34)nc(-c3ccc4c5ccccc5oc5cccc6oc3c4c56)n2)cc1 ; c1ccc(-c2nc(-c3cccc4c3oc3ccccc34)nc(-c3ccc4c5ccccc5oc5cccc6oc3c4c56)n2)cc1 ; c1ccc(-c2nc(-c3ccc4c5ccccc5oc5cccc6oc3c4c56)nc(-c3cccc4oc5ccccc5c34)n2)cc1]

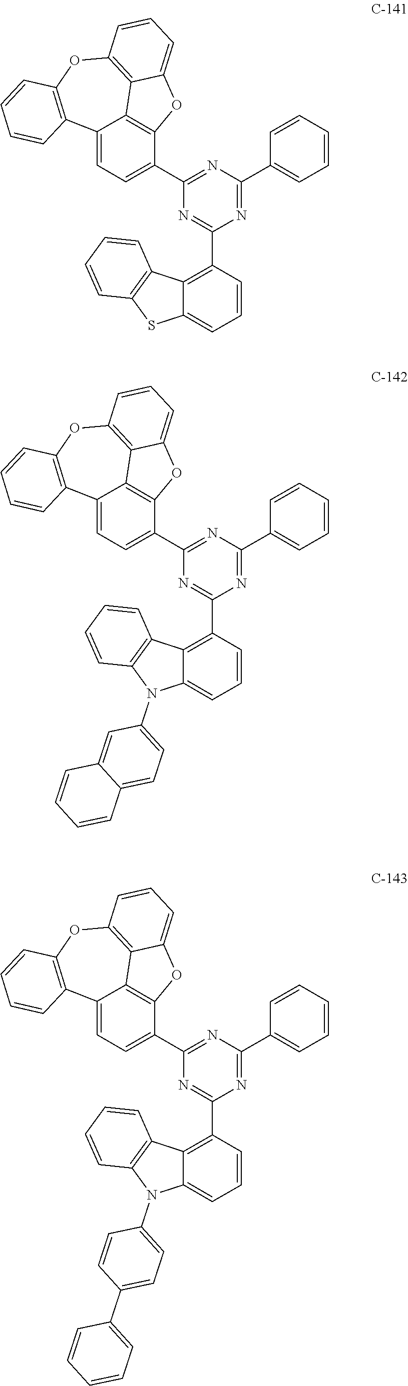 [SMILES: c1ccc(-c2nc(-c3ccc4c5ccccc5oc5cccc6oc3c4c56)nc(-c3cccc4sc5ccccc5c34)n2)cc1 ; c1ccc(-c2nc(-c3ccc4c5ccccc5oc5cccc6oc3c4c56)nc(-c3cccc4c3c3ccccc3n4-c3ccc4ccccc4c3)n2)cc1 ; c1ccc(-c2ccc(-n3c4ccccc4c4c(-c5nc(-c6ccccc6)nc(-c6ccc7c8ccccc8oc8cccc9oc6c7c89)n5)cccc43)cc2)cc1]

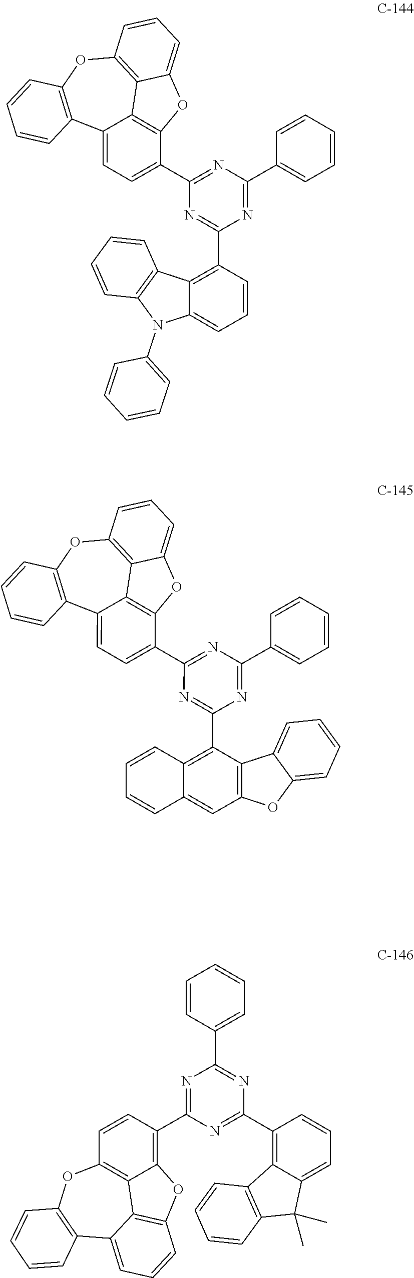 [SMILES: c1ccc(-c2nc(-c3ccc4c5ccccc5oc5cccc6oc3c4c56)nc(-c3cccc4c3c3ccccc3n4-c3ccccc3)n2)cc1 ; c1ccc(-c2nc(-c3ccc4c5ccccc5oc5cccc6oc3c4c56)nc(-c3c4ccccc4cc4oc5ccccc5c34)n2)cc1 ; CC1(C)c2ccccc2-c2c(-c3nc(-c4ccccc4)nc(-c4ccc5oc6ccccc6c6cccc7oc4c5c76)n3)cccc21]

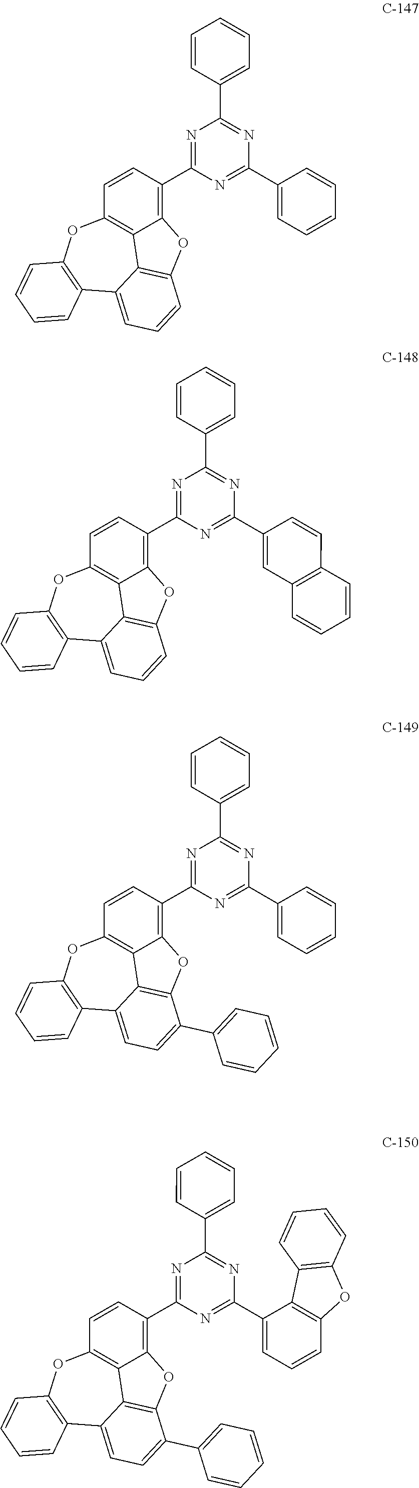 [SMILES: c1ccc(-c2nc(-c3ccccc3)nc(-c3ccc4oc5ccccc5c5cccc6oc3c4c65)n2)cc1 ; c1ccc(-c2nc(-c3ccc4ccccc4c3)nc(-c3ccc4oc5ccccc5c5cccc6oc3c4c65)n2)cc1 ; c1ccc(-c2nc(-c3ccccc3)nc(-c3ccc4oc5ccccc5c5ccc(-c6ccccc6)c6oc3c4c65)n2)cc1 ; c1ccc(-c2nc(-c3ccc4oc5ccccc5c5ccc(-c6ccccc6)c6oc3c4c65)nc(-c3cccc4oc5ccccc5c34)n2)cc1]

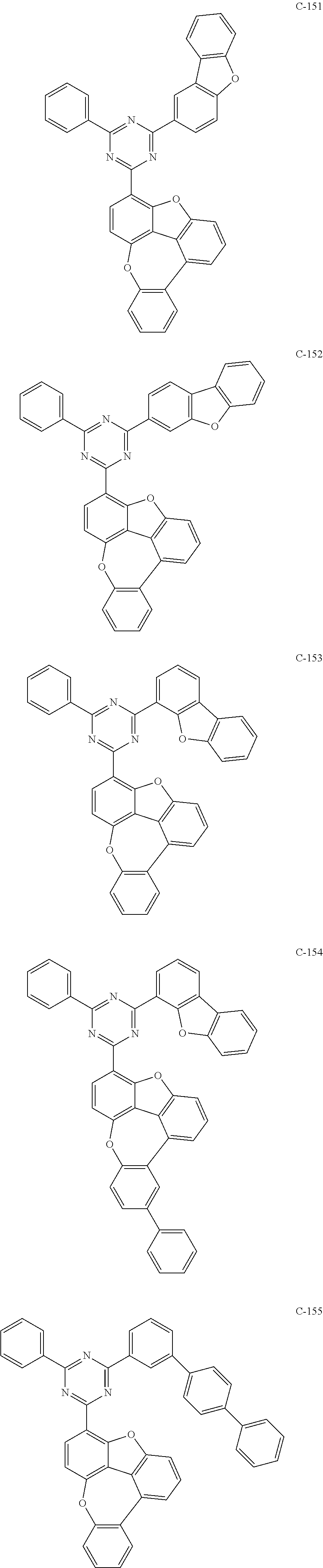 [SMILES: c1ccc(-c2nc(-c3ccc4oc5ccccc5c4c3)nc(-c3ccc4oc5ccccc5c5cccc6oc3c4c65)n2)cc1 ; c1ccc(-c2nc(-c3ccc4c(c3)oc3ccccc34)nc(-c3ccc4oc5ccccc5c5cccc6oc3c4c65)n2)cc1 ; c1ccc(-c2nc(-c3cccc4c3oc3ccccc34)nc(-c3ccc4oc5ccccc5c5cccc6oc3c4c65)n2)cc1 ; c1ccc(-c2ccc3oc4ccc(-c5nc(-c6ccccc6)nc(-c6cccc7c6oc6ccccc67)n5)c5oc6cccc(c3c2)c6c45)cc1 ; c1ccc(-c2ccc(-c3cccc(-c4nc(-c5ccccc5)nc(-c5ccc6oc7ccccc7c7cccc8oc5c6c87)n4)c3)cc2)cc1]

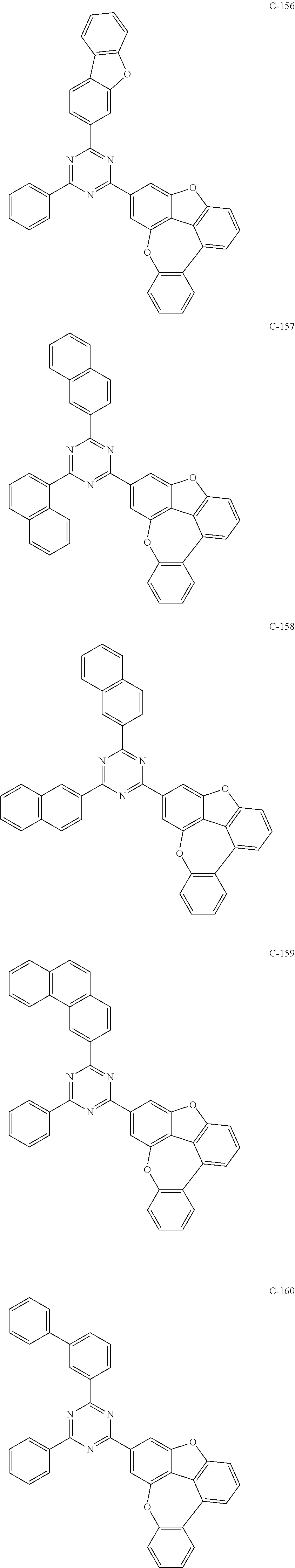 [SMILES: c1ccc(-c2nc(-c3ccc4c(c3)oc3ccccc34)nc(-c3cc4oc5ccccc5c5cccc6oc(c3)c4c65)n2)cc1 ; c1ccc2cc(-c3nc(-c4cc5oc6ccccc6c6cccc7oc(c4)c5c76)nc(-c4cccc5ccccc45)n3)ccc2c1 ; c1ccc2cc(-c3nc(-c4ccc5ccccc5c4)nc(-c4cc5oc6ccccc6c6cccc7oc(c4)c5c76)n3)ccc2c1 ; c1ccc(-c2nc(-c3cc4oc5ccccc5c5cccc6oc(c3)c4c65)nc(-c3ccc4ccc5ccccc5c4c3)n2)cc1 ; c1ccc(-c2cccc(-c3nc(-c4ccccc4)nc(-c4cc5oc6ccccc6c6cccc7oc(c4)c5c76)n3)c2)cc1]

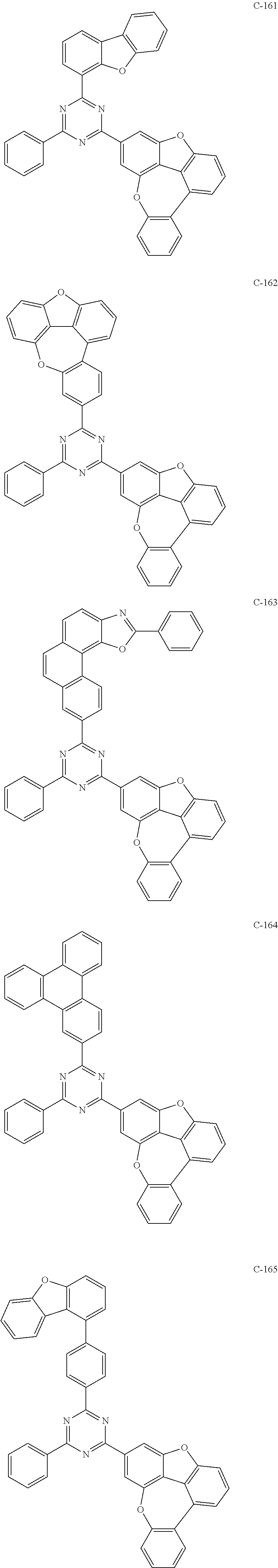

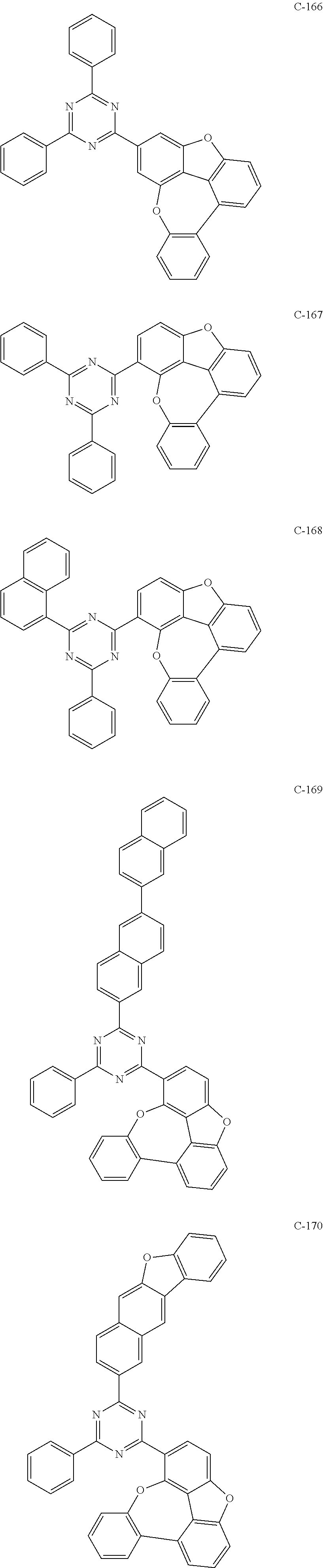 [SMILES: c1ccc(-c2nc(-c3ccccc3)nc(-c3cc4oc5ccccc5c5cccc6oc(c3)c4c65)n2)cc1 ; c1ccc(-c2nc(-c3ccccc3)nc(-c3ccc4oc5cccc6c7ccccc7oc3c4c56)n2)cc1 ; c1ccc(-c2nc(-c3cccc4ccccc34)nc(-c3ccc4oc5cccc6c7ccccc7oc3c4c56)n2)cc1 ; c1ccc(-c2nc(-c3ccc4cc(-c5ccc6ccccc6c5)ccc4c3)nc(-c3ccc4oc5cccc6c7ccccc7oc3c4c56)n2)cc1 ; c1ccc(-c2nc(-c3ccc4cc5oc6ccccc6c5cc4c3)nc(-c3ccc4oc5cccc6c7ccccc7oc3c4c56)n2)cc1]

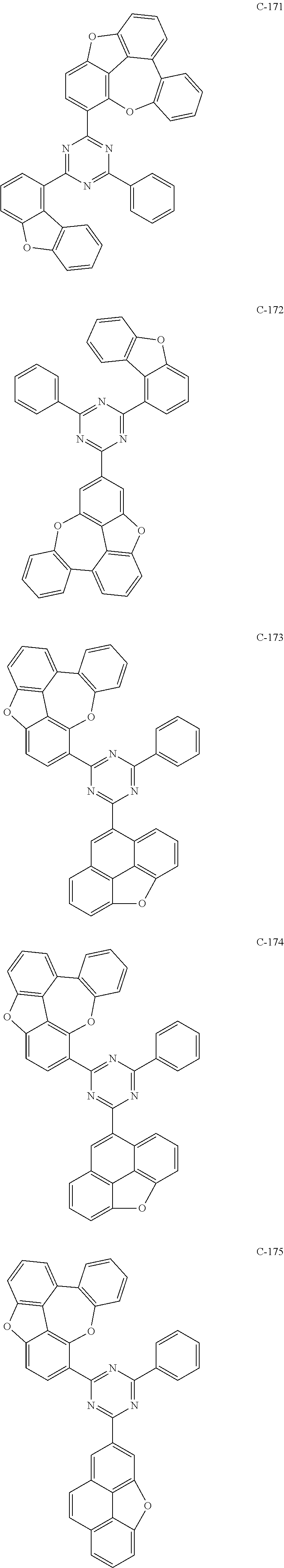 [SMILES: c1ccc(-c2nc(-c3ccc4oc5cccc6c7ccccc7oc3c4c56)nc(-c3cccc4oc5ccccc5c34)n2)cc1 ; c1ccc(-c2nc(-c3cc4oc5ccccc5c5cccc6oc(c3)c4c65)nc(-c3cccc4oc5ccccc5c34)n2)cc1 ; c1ccc(-c2nc(-c3cc4cccc5oc6cccc3c6c45)nc(-c3ccc4oc5cccc6c7ccccc7oc3c4c56)n2)cc1 ; c1ccc(-c2nc(-c3cc4cccc5oc6cccc3c6c45)nc(-c3ccc4oc5cccc6c7ccccc7oc3c4c56)n2)cc1 ; c1ccc(-c2nc(-c3cc4ccc5cccc6oc(c3)c4c56)nc(-c3ccc4oc5cccc6c7ccccc7oc3c4c56)n2)cc1]

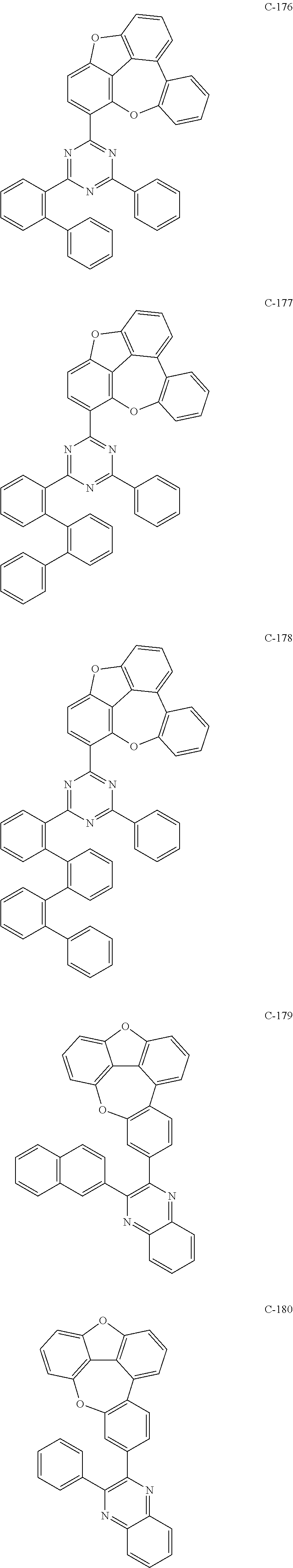 [SMILES: c1ccc(-c2nc(-c3ccccc3-c3ccccc3)nc(-c3ccc4oc5cccc6c7ccccc7oc3c4c56)n2)cc1 ; c1ccc(-c2nc(-c3ccccc3-c3ccccc3-c3ccccc3)nc(-c3ccc4oc5cccc6c7ccccc7oc3c4c56)n2)cc1 ; c1ccc(-c2nc(-c3ccccc3-c3ccccc3-c3ccccc3-c3ccccc3)nc(-c3ccc4oc5cccc6c7ccccc7oc3c4c56)n2)cc1 ; c1ccc2cc(-c3nc4ccccc4nc3-c3ccc4c(c3)oc3cccc5oc6cccc4c6c35)ccc2c1 ; c1ccc(-c2nc3ccccc3nc2-c2ccc3c(c2)oc2cccc4oc5cccc3c5c24)cc1]

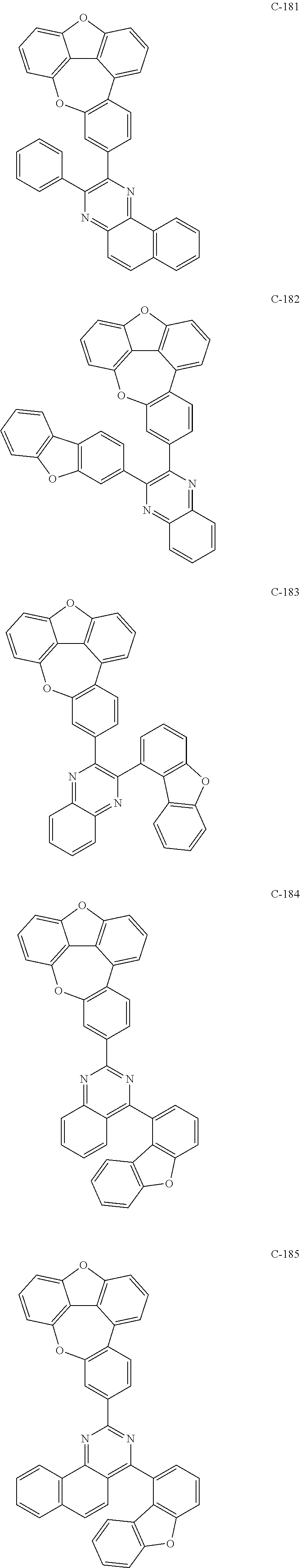 [SMILES: c1ccc(-c2nc3ccc4ccccc4c3nc2-c2ccc3c(c2)oc2cccc4oc5cccc3c5c24)cc1 ; c1ccc2nc(-c3ccc4c(c3)oc3cccc5oc6cccc4c6c35)c(-c3ccc4c(c3)oc3ccccc34)nc2c1 ; c1ccc2nc(-c3cccc4oc5ccccc5c34)c(-c3ccc4c(c3)oc3cccc5oc6cccc4c6c35)nc2c1 ; c1ccc2c(-c3cccc4oc5ccccc5c34)nc(-c3ccc4c(c3)oc3cccc5oc6cccc4c6c35)nc2c1 ; c1ccc2c(c1)ccc1c(-c3cccc4oc5ccccc5c34)nc(-c3ccc4c(c3)oc3cccc5oc6cccc4c6c35)nc12]

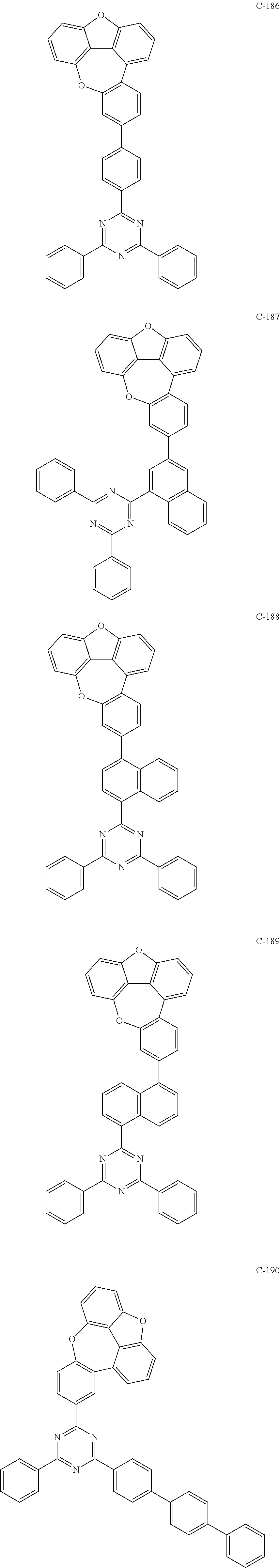

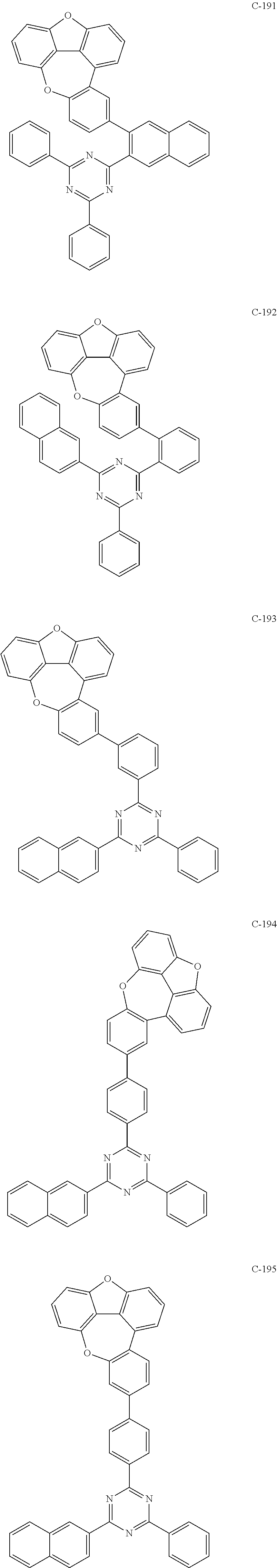 [SMILES: c1ccc(-c2nc(-c3ccccc3)nc(-c3cc4ccccc4cc3-c3ccc4oc5cccc6oc7cccc(c4c3)c7c56)n2)cc1 ; c1ccc(-c2nc(-c3ccc4ccccc4c3)nc(-c3ccccc3-c3ccc4oc5cccc6oc7cccc(c4c3)c7c56)n2)cc1 ; c1ccc(-c2nc(-c3cccc(-c4ccc5oc6cccc7oc8cccc(c5c4)c8c67)c3)nc(-c3ccc4ccccc4c3)n2)cc1 ; c1ccc(-c2nc(-c3ccc(-c4ccc5oc6cccc7oc8cccc(c5c4)c8c67)cc3)nc(-c3ccc4ccccc4c3)n2)cc1 ; c1ccc(-c2nc(-c3ccc(-c4ccc5c(c4)oc4cccc6oc7cccc5c7c46)cc3)nc(-c3ccc4ccccc4c3)n2)cc1]

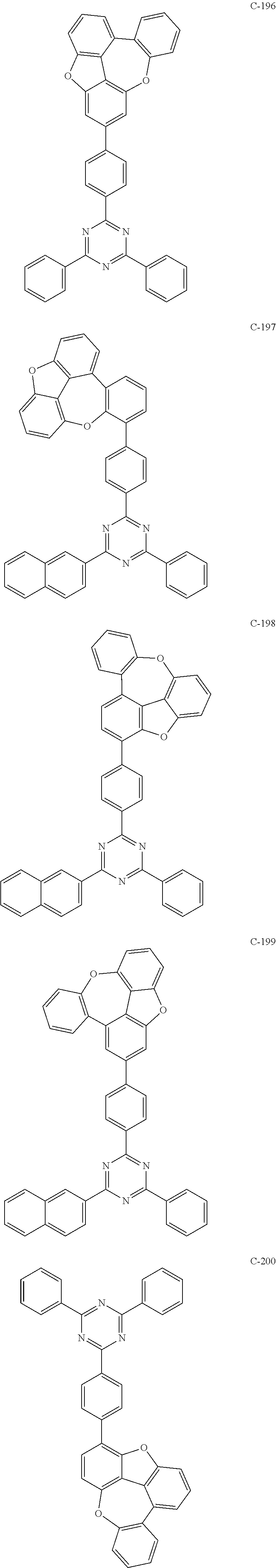 [SMILES: c1ccc(-c2nc(-c3ccccc3)nc(-c3ccc(-c4cc5oc6ccccc6c6cccc7oc(c4)c5c76)cc3)n2)cc1 ; c1ccc(-c2nc(-c3ccc(-c4cccc5c4oc4cccc6oc7cccc5c7c64)cc3)nc(-c3ccc4ccccc4c3)n2)cc1 ; c1ccc(-c2nc(-c3ccc(-c4ccc5c6ccccc6oc6cccc7oc4c5c67)cc3)nc(-c3ccc4ccccc4c3)n2)cc1 ; c1ccc(-c2nc(-c3ccc(-c4cc5oc6cccc7oc8ccccc8c(c4)c5c76)cc3)nc(-c3ccc4ccccc4c3)n2)cc1 ; c1ccc(-c2nc(-c3ccccc3)nc(-c3ccc(-c4ccc5oc6ccccc6c6cccc7oc4c5c76)cc3)n2)cc1]

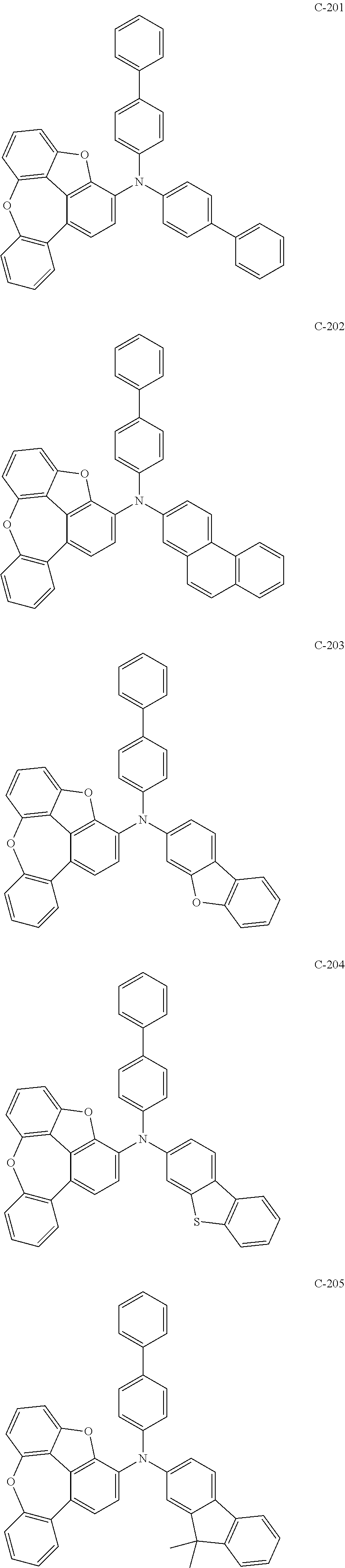 [SMILES: c1ccc(-c2ccc(N(c3ccc(-c4ccccc4)cc3)c3ccc4c5ccccc5oc5cccc6oc3c4c56)cc2)cc1 ; c1ccc(-c2ccc(N(c3ccc4c(ccc5ccccc54)c3)c3ccc4c5ccccc5oc5cccc6oc3c4c56)cc2)cc1 ; c1ccc(-c2ccc(N(c3ccc4c(c3)oc3ccccc34)c3ccc4c5ccccc5oc5cccc6oc3c4c56)cc2)cc1 ; c1ccc(-c2ccc(N(c3ccc4c(c3)sc3ccccc34)c3ccc4c5ccccc5oc5cccc6oc3c4c56)cc2)cc1 ; CC1(C)c2ccccc2-c2ccc(N(c3ccc(-c4ccccc4)cc3)c3ccc4c5ccccc5oc5cccc6oc3c4c56)cc21]

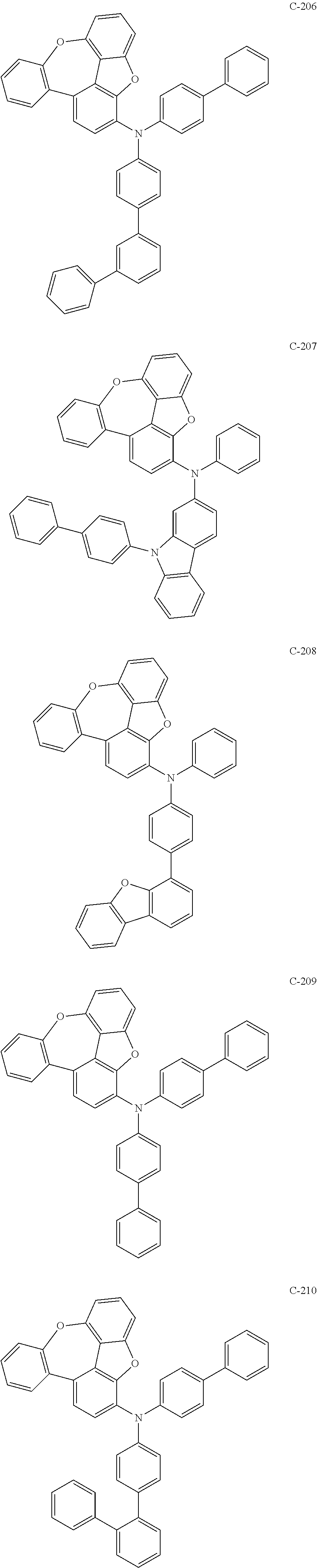 [SMILES: c1ccc(-c2ccc(N(c3ccc(-c4cccc(-c5ccccc5)c4)cc3)c3ccc4c5ccccc5oc5cccc6oc3c4c56)cc2)cc1 ; c1ccc(-c2ccc(-n3c4ccccc4c4ccc(N(c5ccccc5)c5ccc6c7ccccc7oc7cccc8oc5c6c78)cc43)cc2)cc1 ; c1ccc(N(c2ccc(-c3cccc4c3oc3ccccc34)cc2)c2ccc3c4ccccc4oc4cccc5oc2c3c45)cc1 ; c1ccc(-c2ccc(N(c3ccc(-c4ccccc4)cc3)c3ccc4c5ccccc5oc5cccc6oc3c4c56)cc2)cc1 ; c1ccc(-c2ccc(N(c3ccc(-c4ccccc4-c4ccccc4)cc3)c3ccc4c5ccccc5oc5cccc6oc3c4c56)cc2)cc1]

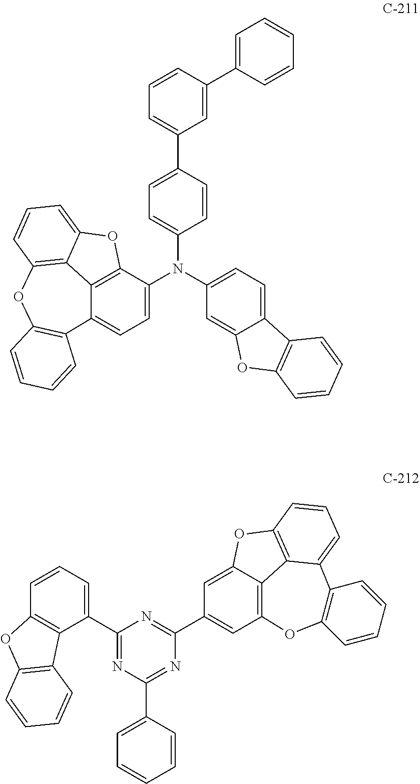 [SMILES: c1ccc(-c2cccc(-c3ccc(N(c4ccc5c(c4)oc4ccccc45)c4ccc5c6ccccc6oc6cccc7oc4c5c67)cc3)c2)cc1 ; c1ccc(-c2nc(-c3cc4oc5ccccc5c5cccc6oc(c3)c4c65)nc(-c3cccc4oc5ccccc5c34)n2)cc1]

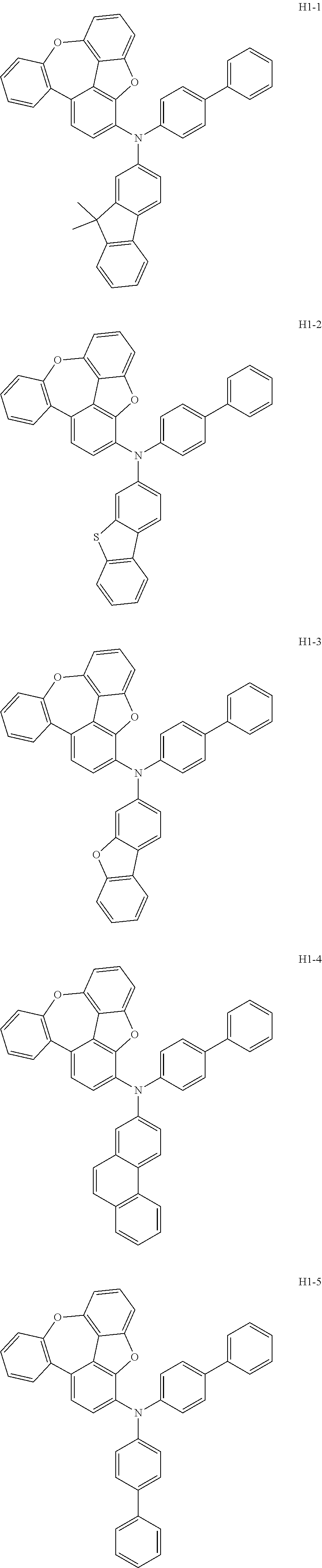 [SMILES: CC1(C)c2ccccc2-c2ccc(N(c3ccc(-c4ccccc4)cc3)c3ccc4c5ccccc5oc5cccc6oc3c4c56)cc21 ; c1ccc(-c2ccc(N(c3ccc4c(c3)sc3ccccc34)c3ccc4c5ccccc5oc5cccc6oc3c4c56)cc2)cc1 ; c1ccc(-c2ccc(N(c3ccc4c(c3)oc3ccccc34)c3ccc4c5ccccc5oc5cccc6oc3c4c56)cc2)cc1 ; c1ccc(-c2ccc(N(c3ccc4c(ccc5ccccc54)c3)c3ccc4c5ccccc5oc5cccc6oc3c4c56)cc2)cc1 ; c1ccc(-c2ccc(N(c3ccc(-c4ccccc4)cc3)c3ccc4c5ccccc5oc5cccc6oc3c4c56)cc2)cc1]

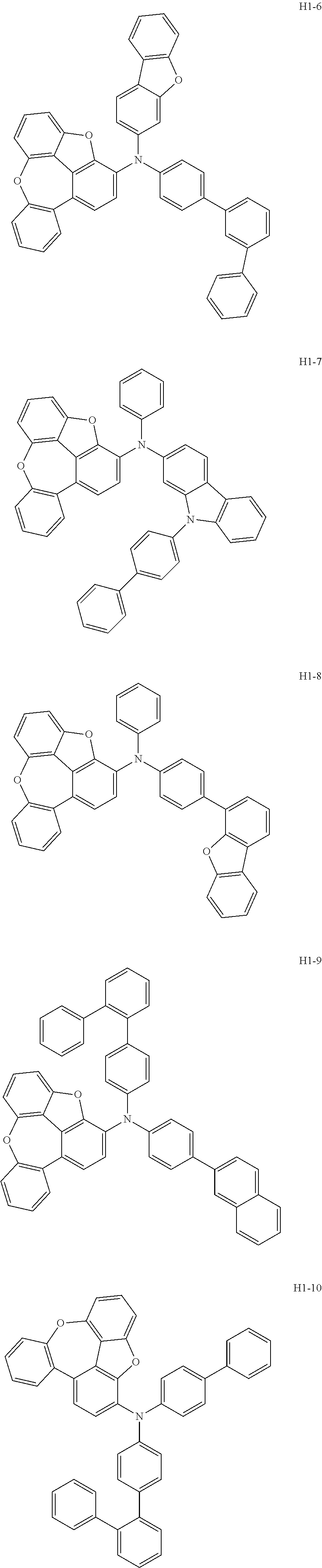 [SMILES: c1ccc(-c2cccc(-c3ccc(N(c4ccc5c(c4)oc4ccccc45)c4ccc5c6ccccc6oc6cccc7oc4c5c67)cc3)c2)cc1 ; c1ccc(-c2ccc(-n3c4ccccc4c4ccc(N(c5ccccc5)c5ccc6c7ccccc7oc7cccc8oc5c6c78)cc43)cc2)cc1 ; c1ccc(N(c2ccc(-c3cccc4c3oc3ccccc34)cc2)c2ccc3c4ccccc4oc4cccc5oc2c3c45)cc1 ; c1ccc(-c2ccccc2-c2ccc(N(c3ccc(-c4ccc5ccccc5c4)cc3)c3ccc4c5ccccc5oc5cccc6oc3c4c56)cc2)cc1 ; c1ccc(-c2ccc(N(c3ccc(-c4ccccc4-c4ccccc4)cc3)c3ccc4c5ccccc5oc5cccc6oc3c4c56)cc2)cc1]

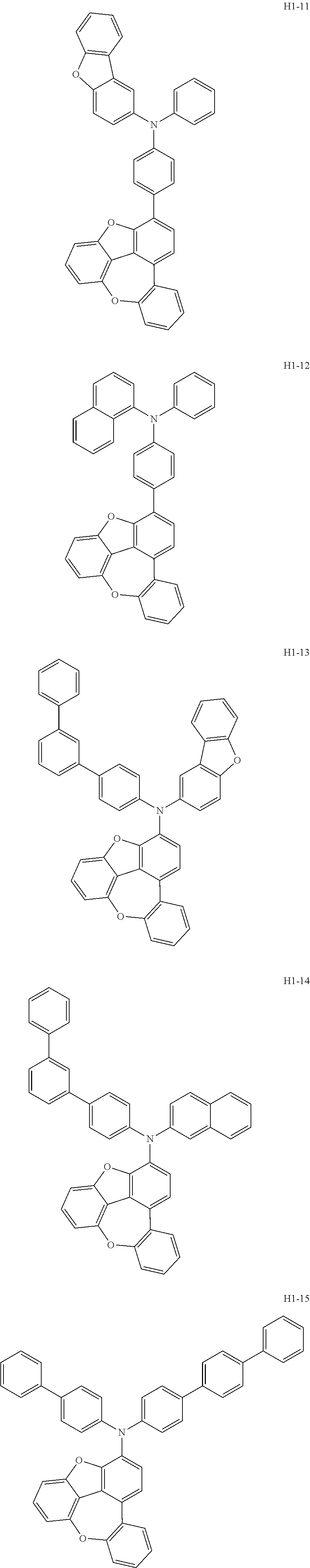 [SMILES: c1ccc(N(c2ccc(-c3ccc4c5ccccc5oc5cccc6oc3c4c56)cc2)c2ccc3oc4ccccc4c3c2)cc1 ; c1ccc(N(c2ccc(-c3ccc4c5ccccc5oc5cccc6oc3c4c56)cc2)c2cccc3ccccc23)cc1 ; c1ccc(-c2cccc(-c3ccc(N(c4ccc5oc6ccccc6c5c4)c4ccc5c6ccccc6oc6cccc7oc4c5c67)cc3)c2)cc1 ; c1ccc(-c2cccc(-c3ccc(N(c4ccc5ccccc5c4)c4ccc5c6ccccc6oc6cccc7oc4c5c67)cc3)c2)cc1 ; c1ccc(-c2ccc(-c3ccc(N(c4ccc(-c5ccccc5)cc4)c4ccc5c6ccccc6oc6cccc7oc4c5c67)cc3)cc2)cc1]

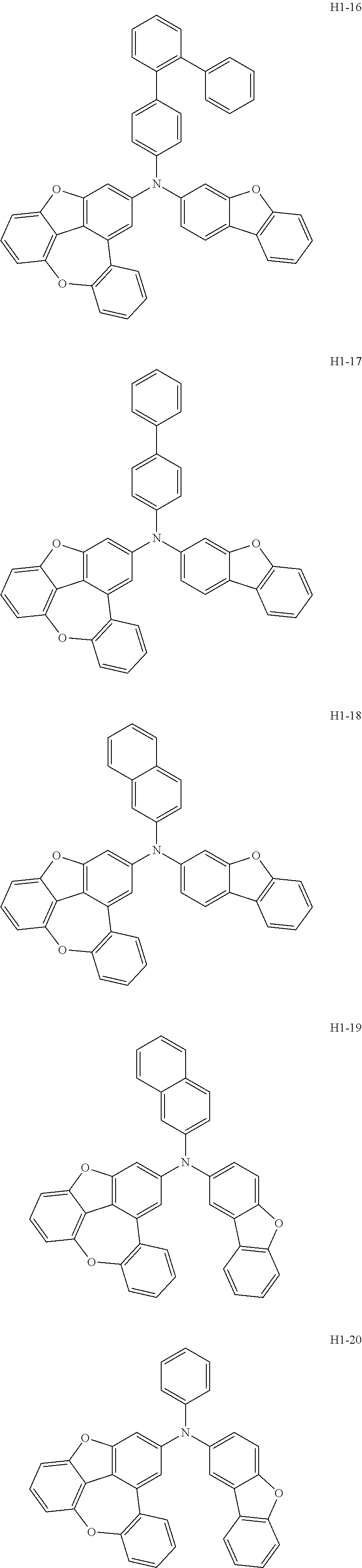 [SMILES: c1ccc(-c2ccccc2-c2ccc(N(c3ccc4c(c3)oc3ccccc34)c3cc4oc5cccc6oc7ccccc7c(c3)c4c65)cc2)cc1 ; c1ccc(-c2ccc(N(c3ccc4c(c3)oc3ccccc34)c3cc4oc5cccc6oc7ccccc7c(c3)c4c65)cc2)cc1 ; c1ccc2cc(N(c3ccc4c(c3)oc3ccccc34)c3cc4oc5cccc6oc7ccccc7c(c3)c4c65)ccc2c1 ; c1ccc2cc(N(c3ccc4oc5ccccc5c4c3)c3cc4oc5cccc6oc7ccccc7c(c3)c4c65)ccc2c1 ; c1ccc(N(c2ccc3oc4ccccc4c3c2)c2cc3oc4cccc5oc6ccccc6c(c2)c3c54)cc1]

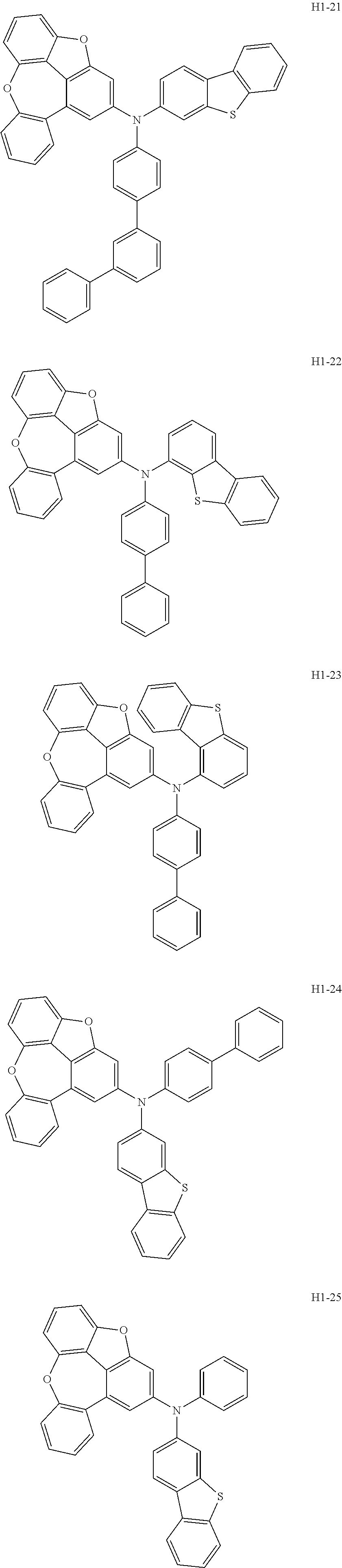 [SMILES: c1ccc(-c2cccc(-c3ccc(N(c4ccc5c(c4)sc4ccccc45)c4cc5oc6cccc7oc8ccccc8c(c4)c5c76)cc3)c2)cc1 ; c1ccc(-c2ccc(N(c3cc4oc5cccc6oc7ccccc7c(c3)c4c65)c3cccc4c3sc3ccccc34)cc2)cc1 ; c1ccc(-c2ccc(N(c3cc4oc5cccc6oc7ccccc7c(c3)c4c65)c3cccc4sc5ccccc5c34)cc2)cc1 ; c1ccc(-c2ccc(N(c3ccc4c(c3)sc3ccccc34)c3cc4oc5cccc6oc7ccccc7c(c3)c4c65)cc2)cc1 ; c1ccc(N(c2ccc3c(c2)sc2ccccc23)c2cc3oc4cccc5oc6ccccc6c(c2)c3c54)cc1]

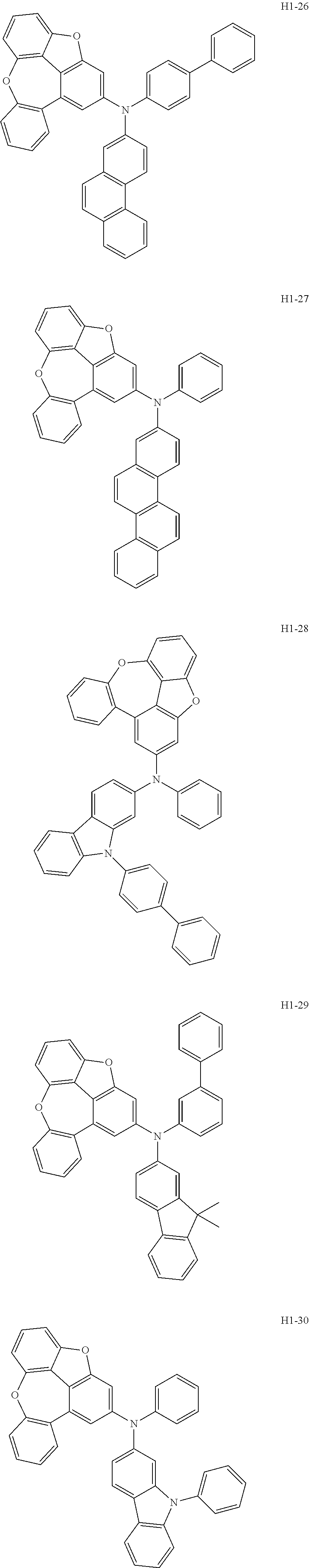 [SMILES: c1ccc(-c2ccc(N(c3ccc4c(ccc5ccccc54)c3)c3cc4oc5cccc6oc7ccccc7c(c3)c4c65)cc2)cc1 ; c1ccc(N(c2ccc3c(ccc4c5ccccc5ccc34)c2)c2cc3oc4cccc5oc6ccccc6c(c2)c3c54)cc1 ; c1ccc(-c2ccc(-n3c4ccccc4c4ccc(N(c5ccccc5)c5cc6oc7cccc8oc9ccccc9c(c5)c6c87)cc43)cc2)cc1 ; CC1(C)c2ccccc2-c2ccc(N(c3cccc(-c4ccccc4)c3)c3cc4oc5cccc6oc7ccccc7c(c3)c4c65)cc21 ; c1ccc(N(c2cc3oc4cccc5oc6ccccc6c(c2)c3c54)c2ccc3c4ccccc4n(-c4ccccc4)c3c2)cc1]

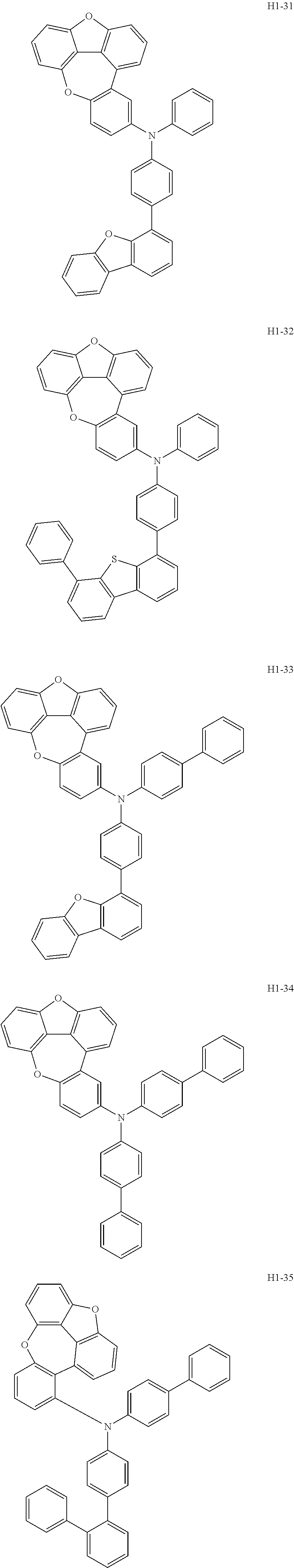 [SMILES: c1ccc(N(c2ccc(-c3cccc4c3oc3ccccc34)cc2)c2ccc3oc4cccc5oc6cccc(c3c2)c6c45)cc1 ; c1ccc(-c2cccc3c2sc2c(-c4ccc(N(c5ccccc5)c5ccc6oc7cccc8oc9cccc(c6c5)c9c78)cc4)cccc23)cc1 ; c1ccc(-c2ccc(N(c3ccc(-c4cccc5c4oc4ccccc45)cc3)c3ccc4oc5cccc6oc7cccc(c4c3)c7c56)cc2)cc1 ; c1ccc(-c2ccc(N(c3ccc(-c4ccccc4)cc3)c3ccc4oc5cccc6oc7cccc(c4c3)c7c56)cc2)cc1 ; c1ccc(-c2ccc(N(c3ccc(-c4ccccc4-c4ccccc4)cc3)c3cccc4oc5cccc6oc7cccc(c34)c7c56)cc2)cc1]

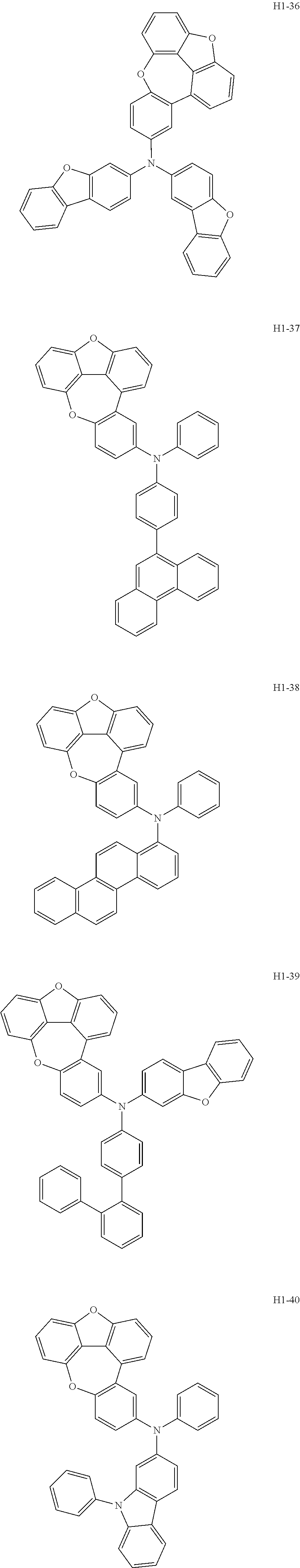 [SMILES: c1ccc2c(c1)oc1cc(N(c3ccc4oc5ccccc5c4c3)c3ccc4oc5cccc6oc7cccc(c4c3)c7c56)ccc12 ; c1ccc(N(c2ccc(-c3cc4ccccc4c4ccccc34)cc2)c2ccc3oc4cccc5oc6cccc(c3c2)c6c45)cc1 ; c1ccc(N(c2ccc3oc4cccc5oc6cccc(c3c2)c6c45)c2cccc3c2ccc2c4ccccc4ccc32)cc1 ; c1ccc(-c2ccccc2-c2ccc(N(c3ccc4c(c3)oc3ccccc34)c3ccc4oc5cccc6oc7cccc(c4c3)c7c56)cc2)cc1 ; c1ccc(N(c2ccc3oc4cccc5oc6cccc(c3c2)c6c45)c2ccc3c4ccccc4n(-c4ccccc4)c3c2)cc1]

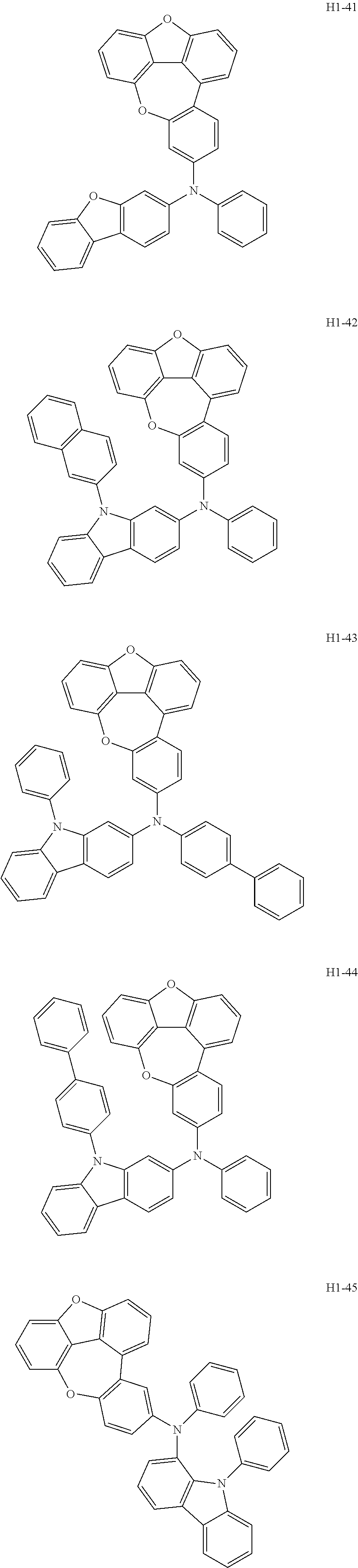 [SMILES: c1ccc(N(c2ccc3c(c2)oc2ccccc23)c2ccc3c(c2)oc2cccc4oc5cccc3c5c24)cc1 ; c1ccc(N(c2ccc3c(c2)oc2cccc4oc5cccc3c5c24)c2ccc3c4ccccc4n(-c4ccc5ccccc5c4)c3c2)cc1 ; c1ccc(-c2ccc(N(c3ccc4c(c3)oc3cccc5oc6cccc4c6c35)c3ccc4c5ccccc5n(-c5ccccc5)c4c3)cc2)cc1 ; c1ccc(-c2ccc(-n3c4ccccc4c4ccc(N(c5ccccc5)c5ccc6c(c5)oc5cccc7oc8cccc6c8c57)cc43)cc2)cc1 ; c1ccc(N(c2ccc3oc4cccc5oc6cccc(c3c2)c6c45)c2cccc3c4ccccc4n(-c4ccccc4)c23)cc1]

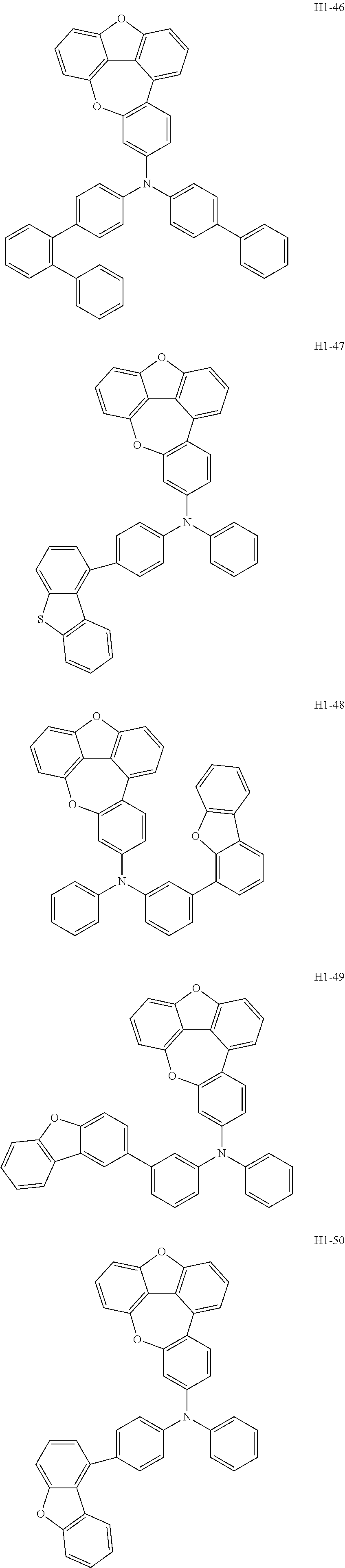 [SMILES: c1ccc(-c2ccc(N(c3ccc(-c4ccccc4-c4ccccc4)cc3)c3ccc4c(c3)oc3cccc5oc6cccc4c6c35)cc2)cc1 ; c1ccc(N(c2ccc(-c3cccc4sc5ccccc5c34)cc2)c2ccc3c(c2)oc2cccc4oc5cccc3c5c24)cc1 ; c1ccc(N(c2cccc(-c3cccc4c3oc3ccccc34)c2)c2ccc3c(c2)oc2cccc4oc5cccc3c5c24)cc1 ; c1ccc(N(c2cccc(-c3ccc4oc5ccccc5c4c3)c2)c2ccc3c(c2)oc2cccc4oc5cccc3c5c24)cc1 ; c1ccc(N(c2ccc(-c3cccc4oc5ccccc5c34)cc2)c2ccc3c(c2)oc2cccc4oc5cccc3c5c24)cc1]

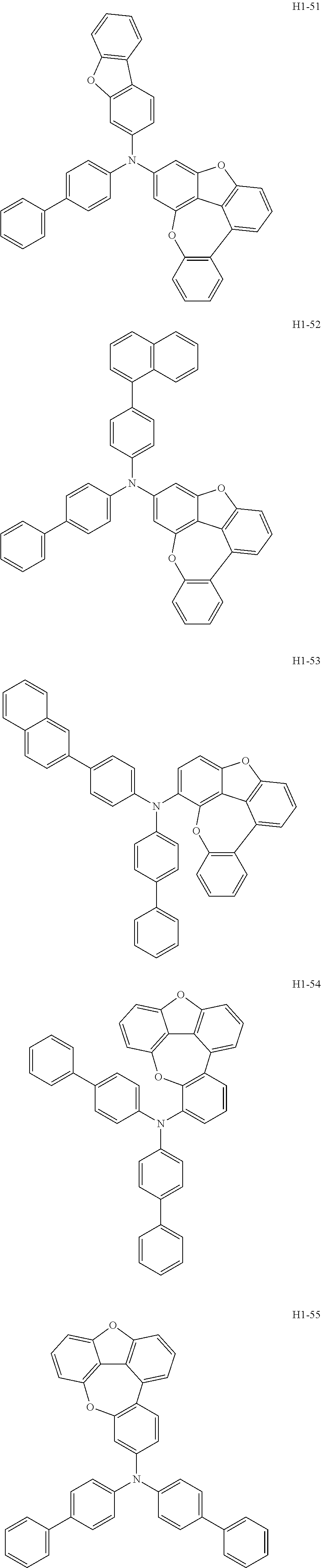

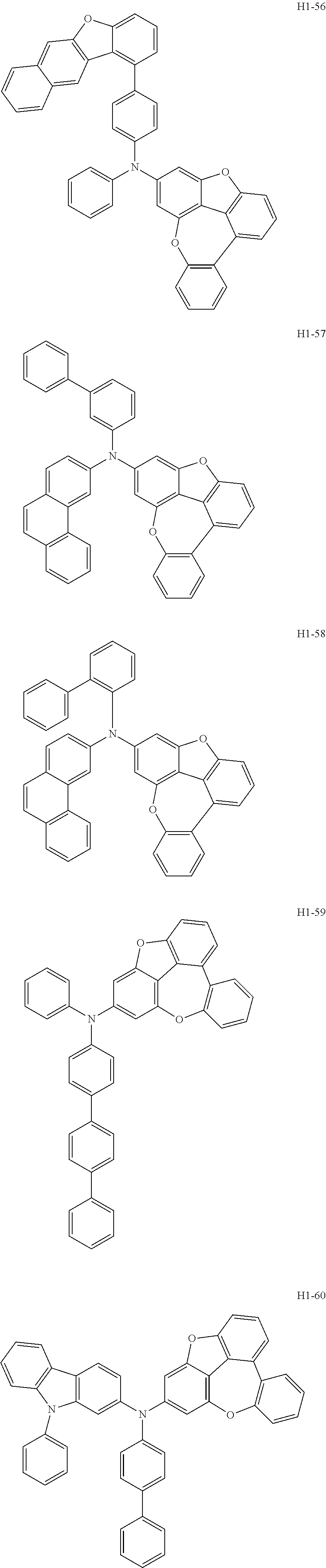 [SMILES: c1ccc(N(c2ccc(-c3cccc4oc5cc6ccccc6cc5c34)cc2)c2cc3oc4ccccc4c4cccc5oc(c2)c3c54)cc1 ; c1ccc(-c2cccc(N(c3cc4oc5ccccc5c5cccc6oc(c3)c4c65)c3ccc4ccc5ccccc5c4c3)c2)cc1 ; c1ccc(-c2ccccc2N(c2cc3oc4ccccc4c4cccc5oc(c2)c3c54)c2ccc3ccc4ccccc4c3c2)cc1 ; c1ccc(-c2ccc(-c3ccc(N(c4ccccc4)c4cc5oc6ccccc6c6cccc7oc(c4)c5c76)cc3)cc2)cc1 ; c1ccc(-c2ccc(N(c3cc4oc5ccccc5c5cccc6oc(c3)c4c65)c3ccc4c5ccccc5n(-c5ccccc5)c4c3)cc2)cc1]

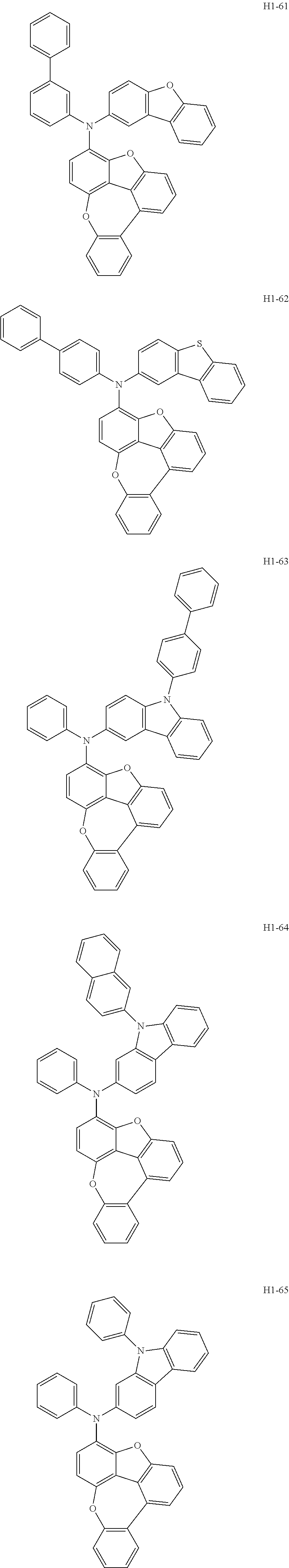 [SMILES: c1ccc(-c2cccc(N(c3ccc4oc5ccccc5c4c3)c3ccc4oc5ccccc5c5cccc6oc3c4c65)c2)cc1 ; c1ccc(-c2ccc(N(c3ccc4sc5ccccc5c4c3)c3ccc4oc5ccccc5c5cccc6oc3c4c65)cc2)cc1 ; c1ccc(-c2ccc(-n3c4ccccc4c4cc(N(c5ccccc5)c5ccc6oc7ccccc7c7cccc8oc5c6c87)ccc43)cc2)cc1 ; c1ccc(N(c2ccc3c4ccccc4n(-c4ccc5ccccc5c4)c3c2)c2ccc3oc4ccccc4c4cccc5oc2c3c54)cc1 ; c1ccc(N(c2ccc3c4ccccc4n(-c4ccccc4)c3c2)c2ccc3oc4ccccc4c4cccc5oc2c3c54)cc1]

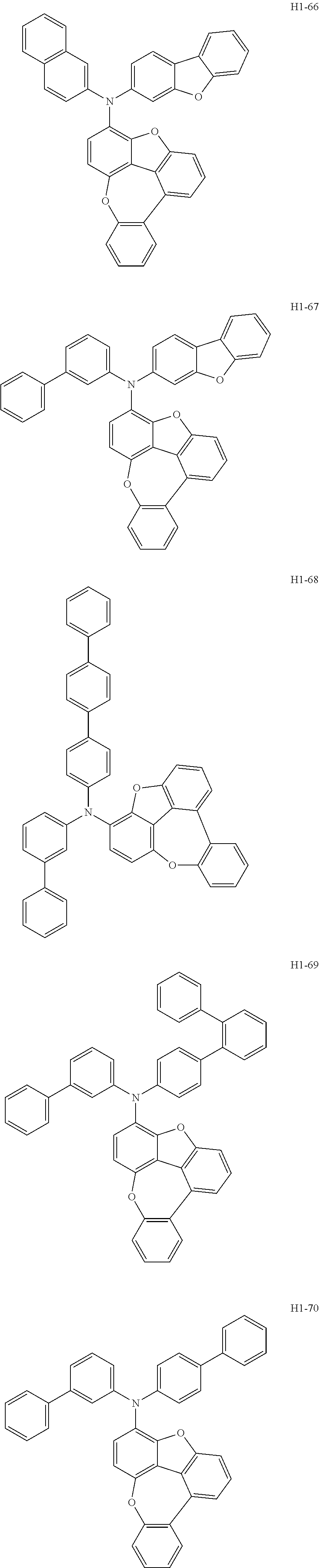 [SMILES: c1ccc2cc(N(c3ccc4c(c3)oc3ccccc34)c3ccc4oc5ccccc5c5cccc6oc3c4c65)ccc2c1 ; c1ccc(-c2cccc(N(c3ccc4c(c3)oc3ccccc34)c3ccc4oc5ccccc5c5cccc6oc3c4c65)c2)cc1 ; c1ccc(-c2ccc(-c3ccc(N(c4cccc(-c5ccccc5)c4)c4ccc5oc6ccccc6c6cccc7oc4c5c76)cc3)cc2)cc1 ; c1ccc(-c2cccc(N(c3ccc(-c4ccccc4-c4ccccc4)cc3)c3ccc4oc5ccccc5c5cccc6oc3c4c65)c2)cc1 ; c1ccc(-c2ccc(N(c3cccc(-c4ccccc4)c3)c3ccc4oc5ccccc5c5cccc6oc3c4c65)cc2)cc1]

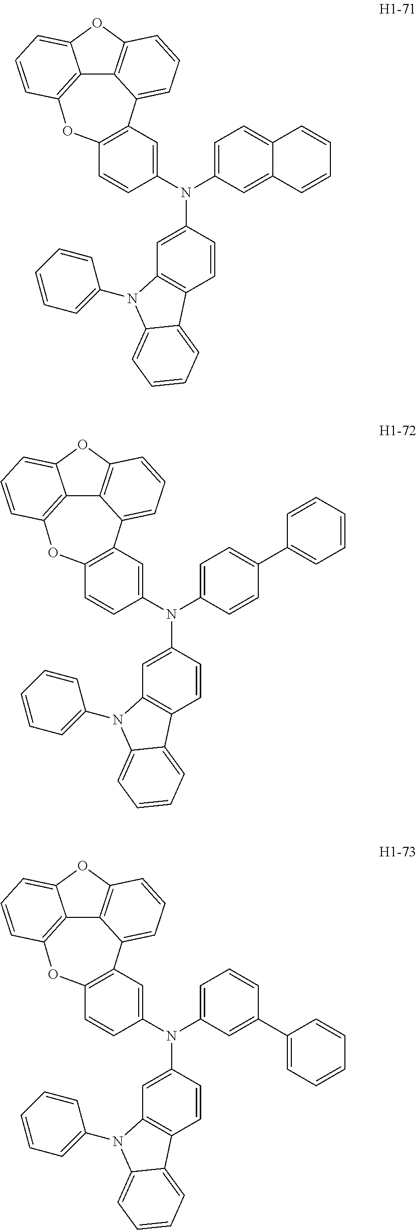 [SMILES: c1ccc(-n2c3ccccc3c3ccc(N(c4ccc5ccccc5c4)c4ccc5oc6cccc7oc8cccc(c5c4)c8c67)cc32)cc1 ; c1ccc(-c2ccc(N(c3ccc4oc5cccc6oc7cccc(c4c3)c7c56)c3ccc4c5ccccc5n(-c5ccccc5)c4c3)cc2)cc1 ; c1ccc(-c2cccc(N(c3ccc4oc5cccc6oc7cccc(c4c3)c7c56)c3ccc4c5ccccc5n(-c5ccccc5)c4c3)c2)cc1]

The present disclosure provides a plurality of host materials comprising a compound represented by Formula 1 and a compound represented by Formula 4 or 5. In addition, the present disclosure provides an organic electroluminescent device comprising a first electrode; a second electrode; and at least one light-emitting layer between the first electrode and the second electrode, wherein the at least one light-emitting layer comprises a plurality of host materials according to the present disclosure.

In Formula 4, T represents O or S.

In Formula 4, K₁ to K₃, each independently, represent N or CR₂₃, with the proviso that at least one of K₁ to K₃ represents N. According to one embodiment of the present disclosure, at least two of K₁ to K₃ represents N. According to another embodiment of the present disclosure, all of K₁ to K₃ represent N.

In Formula 4, L₃ to L₅, each independently, represent a single bond, a substituted or unsubstituted (C6-C30)arylene, a substituted or unsubstituted (C3-C30)cycloalkylene, or a substituted or unsubstituted (3- to 30 membered)heteroarylene. According to one embodiment of the present disclosure, L₃ to L₅, each independently, represent a single bond, a substituted or unsubstituted (C6-C25)arylene, or a substituted or unsubstituted (5- to 25-membered)heteroarylene. According to another embodiment of the present disclosure, L₃ to L₅, each independently, represent a single bond, a (C6-C18)arylene unsubstituted or substituted with deuterium, or a (5- to 20-membered)heteroarylene unsubstituted or substituted with deuterium. For example, L₃ to L₅, each independently, represent a single bond, a phenylene, a naphthylene, a biphenylene, a naphthylenephenylene, a phenylenenaphthylene, or a dibenzofuranylene, etc., which may be substituted with deuterium.

In Formula 4, R₂₁ to R₂₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), or —N—(R′)(R″); or may be linked to an adjacent substituent(s) to form a ring(s). According to one embodiment of the present disclosure, R₂₁ and R₂₂, each independently, represent hydrogen, deuterium, a substituted or unsubstituted (C6-C25)aryl, or a substituted or unsubstituted (5- to 25-membered)heteroaryl. According to another embodiment of the present disclosure, R₂₁ and R₂₂, each independently, represent hydrogen, deuterium, or a (C6-C18)aryl unsubstituted or substituted with deuterium. For example, R₂₁ and R₂₂, each independently, may be hydrogen, deuterium, a phenyl, a naphthyl, a biphenyl, a naphthylphenyl, a phenylnaphthyl, or a phenanthrenyl, etc., which may be substituted with deuterium.

In Formula 4, Ar₃ and Ar₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), or —N—(R′)(R″); or may be linked to an adjacent substituent(s) to form a ring(s). R′ and R″, each independently, represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl. According to one embodiment of the present disclosure, Ar₃ and Ar₄, each independently, represent a substituted or unsubstituted (C6-C25)aryl, or a substituted or unsubstituted (5- to 25-membered)heteroaryl. According to another embodiment of the present disclosure, Ar₃ and Ar₄, each independently, represent a substituted or unsubstituted (C6-C18)aryl, or a substituted or unsubstituted (5- to 20-membered)heteroaryl, which may be substituted with at least one of deuterium and a (C6-C30)aryl(s). For example, Ar₃ and Ar₄, each independently, may be a phenyl; a naphthyl; a biphenyl; a naphthylphenyl; a phenylnaphthyl; a phenanthrenyl; a terphenyl; a chrysenyl; a benzo[c]phenanthryl; a dibenzofuranyl unsubstituted or substituted with at least one of a phenyl(s), a naphthyl(s) and a biphenyl(s); a benzonaphthofuranyl; a benzophenanthrofuranyl; a phenanthrooxazolyl substituted with a phenyl(s); etc., which may be substituted with deuterium.

In Formula 4, a represents an integer of 1 to 4, b represents an integer of 1 to 3, c represents an integer of 1 or 2, and if a to c, respectively, represent an integer of 2 or more, each of R₂₁, each of R₂₂, and each of L₃ may be the same as or different from each other.

Formula 4 may be represented by any one of the following Formulas 4-1 to 4-4.

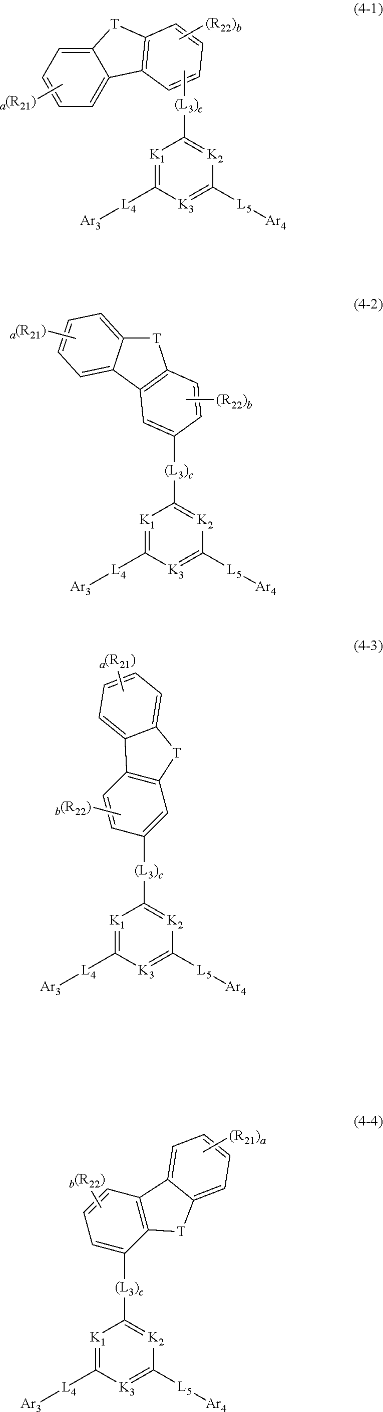

In Formulas 4-1 to 4-4, T, K₁ to K₃, L₃ to L₅, R₂₁, R₂₂, Ar₃, Ar₄, a, b, and c are as defined in Formula 4.

The compound represented by Formula 4 may be at least one selected from the group consisting of the following compounds, but is not limited thereto.

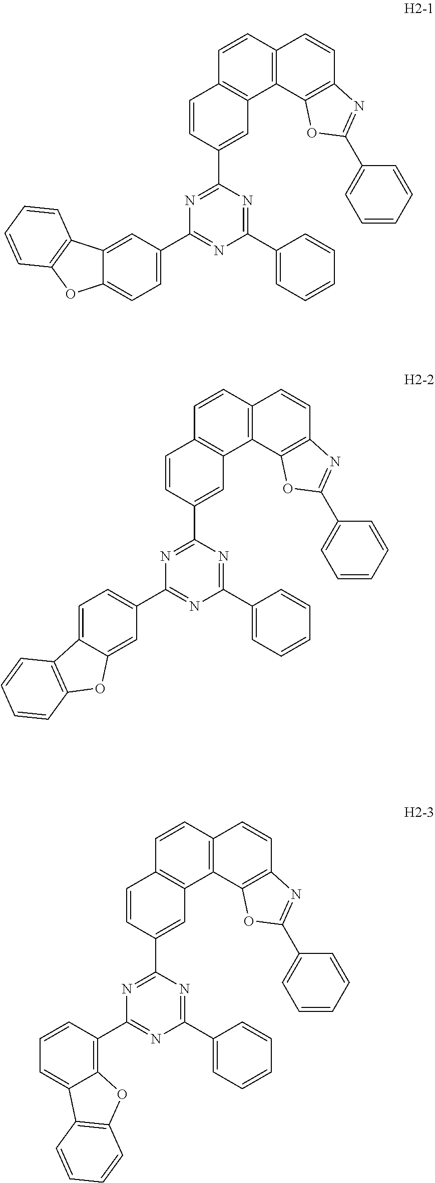 [SMILES: c1ccc(-c2nc(-c3ccc4oc5ccccc5c4c3)nc(-c3ccc4ccc5ccc6nc(-c7ccccc7)oc6c5c4c3)n2)cc1 ; c1ccc(-c2nc(-c3ccc4c(c3)oc3ccccc34)nc(-c3ccc4ccc5ccc6nc(-c7ccccc7)oc6c5c4c3)n2)cc1 ; c1ccc(-c2nc(-c3ccc4ccc5ccc6nc(-c7ccccc7)oc6c5c4c3)nc(-c3cccc4c3oc3ccccc34)n2)cc1]

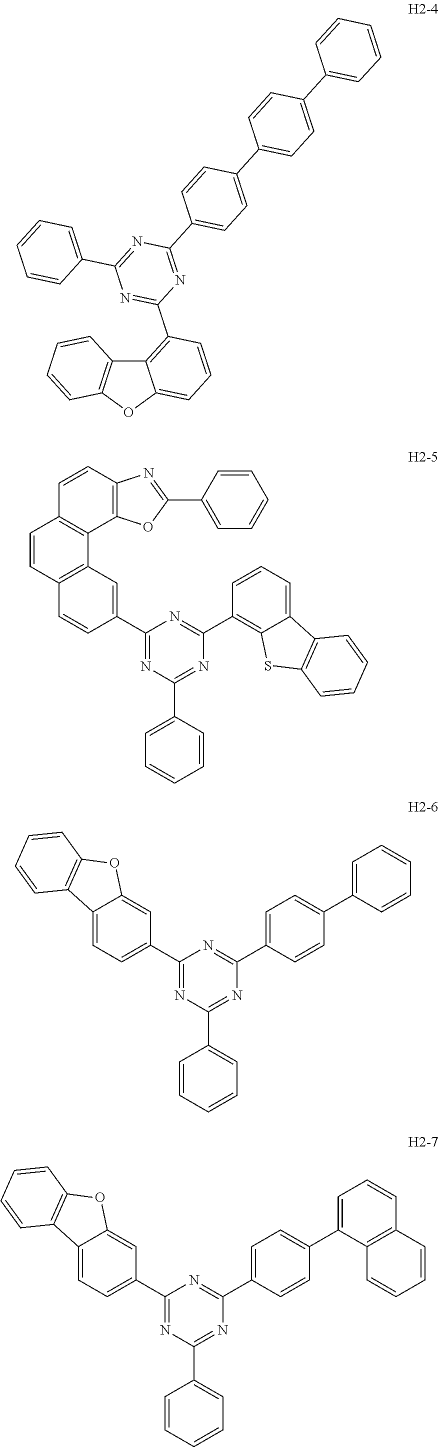 [SMILES: c1ccc(-c2ccc(-c3ccc(-c4nc(-c5ccccc5)nc(-c5cccc6oc7ccccc7c56)n4)cc3)cc2)cc1 ; c1ccc(-c2nc(-c3ccc4ccc5ccc6nc(-c7ccccc7)oc6c5c4c3)nc(-c3cccc4c3sc3ccccc34)n2)cc1 ; c1ccc(-c2ccc(-c3nc(-c4ccccc4)nc(-c4ccc5c(c4)oc4ccccc45)n3)cc2)cc1 ; c1ccc(-c2nc(-c3ccc(-c4cccc5ccccc45)cc3)nc(-c3ccc4c(c3)oc3ccccc34)n2)cc1]

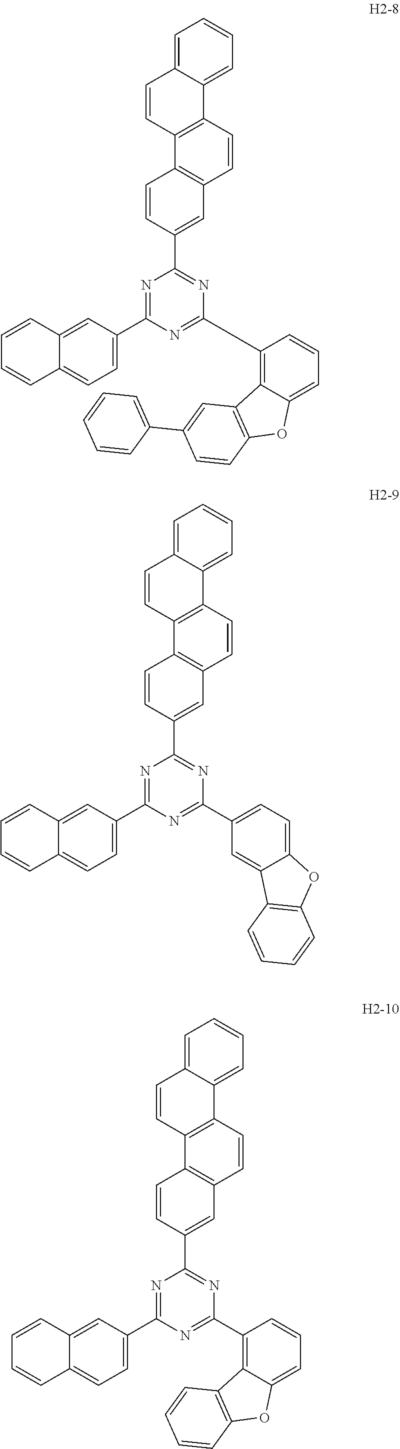

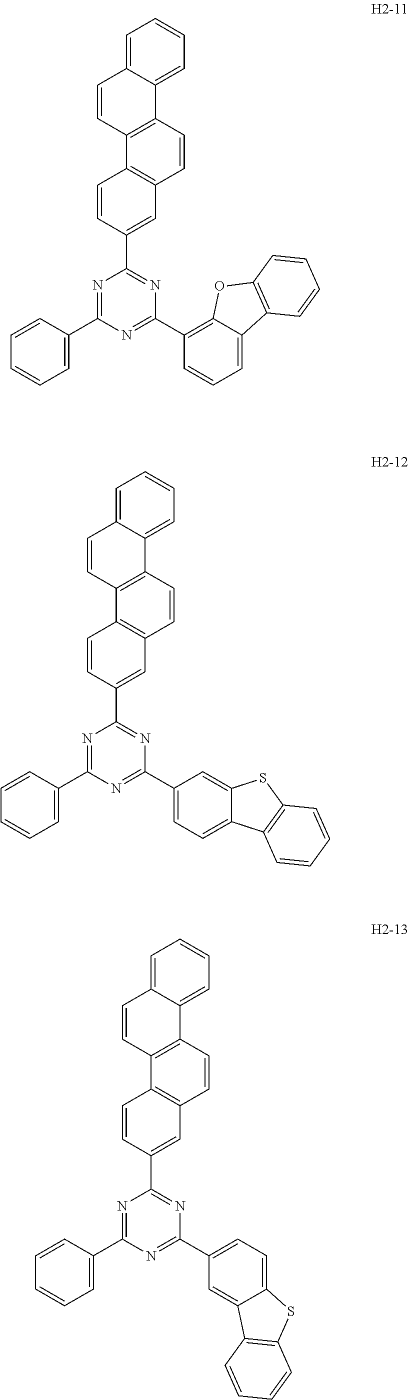 [SMILES: c1ccc(-c2nc(-c3ccc4c(ccc5c6ccccc6ccc45)c3)nc(-c3cccc4c3oc3ccccc34)n2)cc1 ; c1ccc(-c2nc(-c3ccc4c(ccc5c6ccccc6ccc45)c3)nc(-c3ccc4c(c3)sc3ccccc34)n2)cc1 ; c1ccc(-c2nc(-c3ccc4c(ccc5c6ccccc6ccc45)c3)nc(-c3ccc4sc5ccccc5c4c3)n2)cc1]

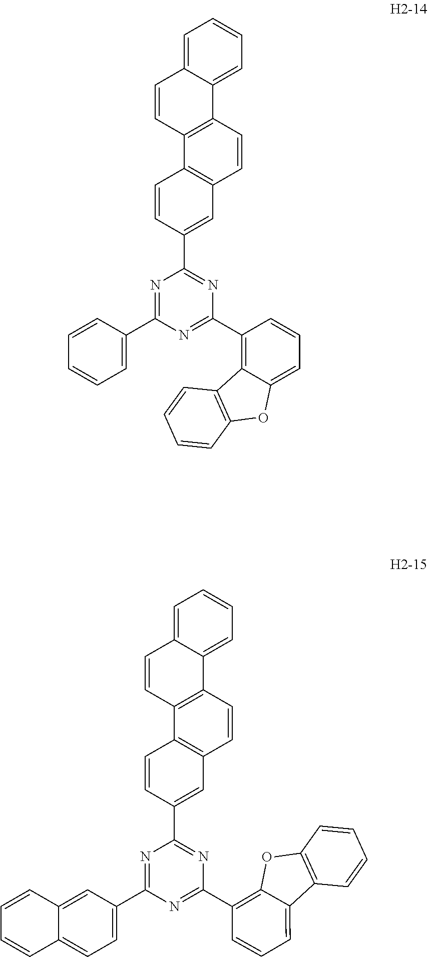 [SMILES: c1ccc(-c2nc(-c3ccc4c(ccc5c6ccccc6ccc45)c3)nc(-c3cccc4oc5ccccc5c34)n2)cc1 ; c1ccc2cc(-c3nc(-c4ccc5c(ccc6c7ccccc7ccc56)c4)nc(-c4cccc5c4oc4ccccc45)n3)ccc2c1]

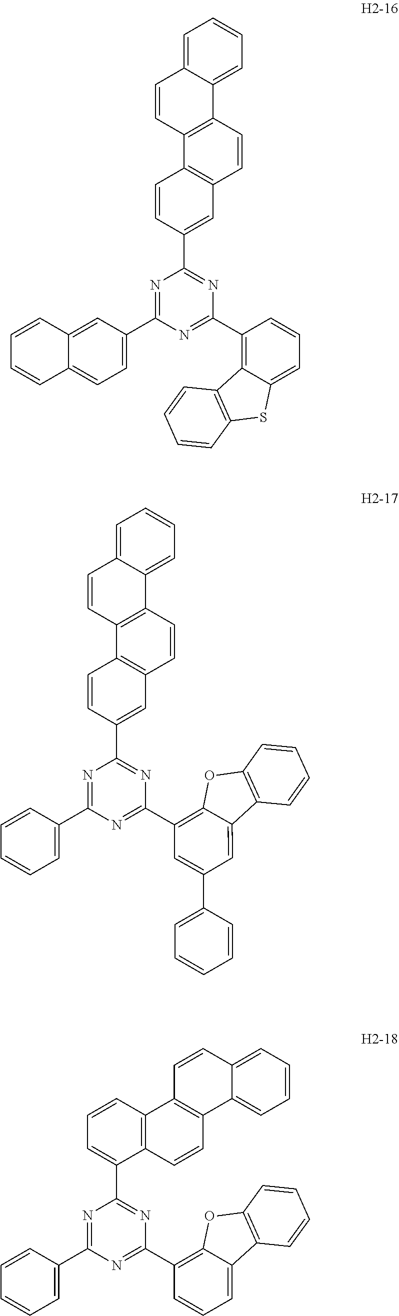 [SMILES: c1ccc2cc(-c3nc(-c4ccc5c(ccc6c7ccccc7ccc56)c4)nc(-c4cccc5sc6ccccc6c45)n3)ccc2c1 ; c1ccc(-c2cc(-c3nc(-c4ccccc4)nc(-c4ccc5c(ccc6c7ccccc7ccc56)c4)n3)c3oc4ccccc4c3c2)cc1 ; c1ccc(-c2nc(-c3cccc4c3ccc3c5ccccc5ccc43)nc(-c3cccc4c3oc3ccccc34)n2)cc1]

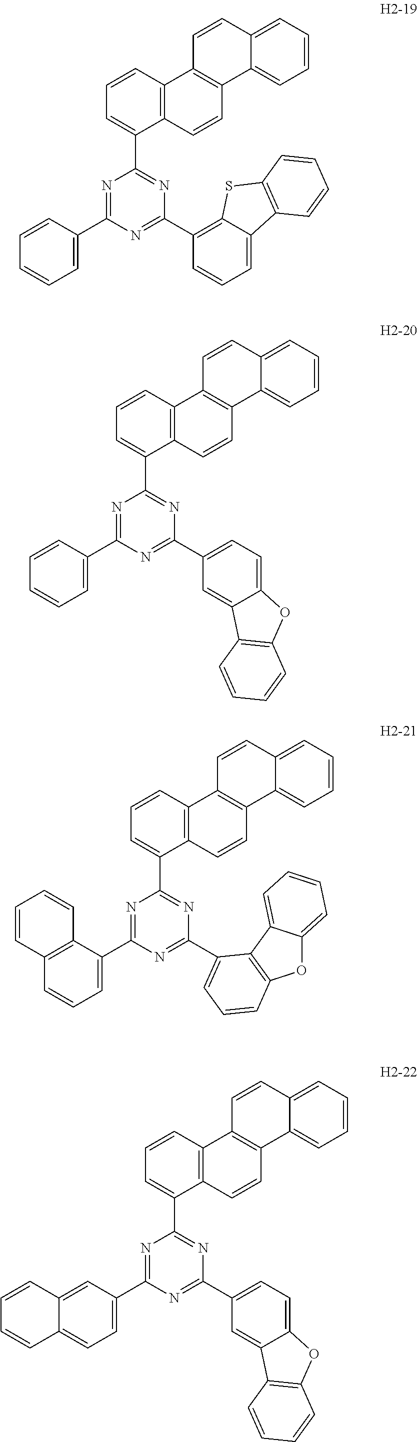

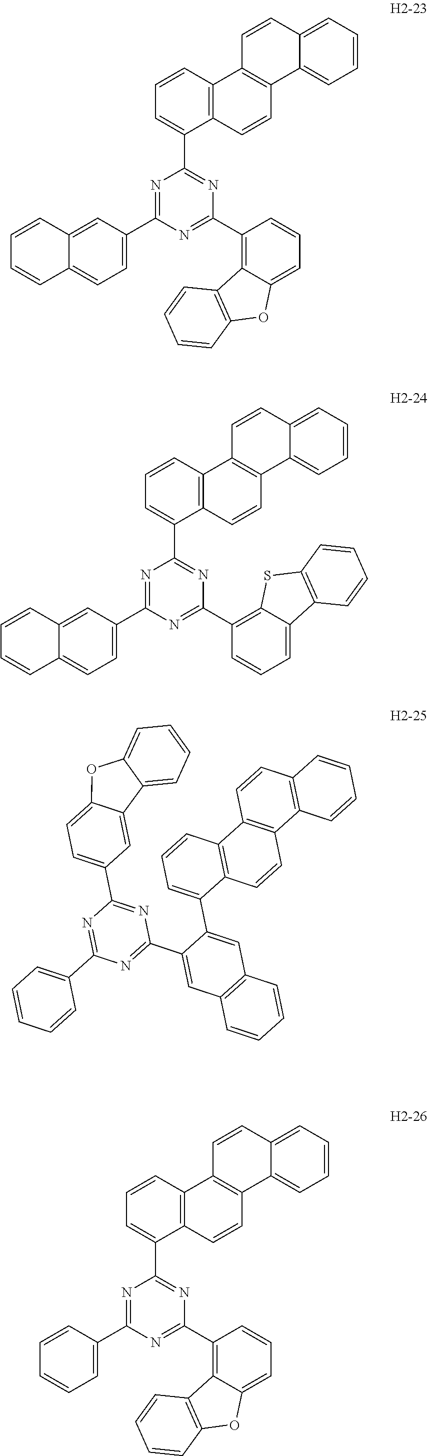 [SMILES: c1ccc2cc(-c3nc(-c4cccc5c4ccc4c6ccccc6ccc54)nc(-c4cccc5oc6ccccc6c45)n3)ccc2c1 ; c1ccc2cc(-c3nc(-c4cccc5c4ccc4c6ccccc6ccc54)nc(-c4cccc5c4sc4ccccc45)n3)ccc2c1 ; c1ccc(-c2nc(-c3ccc4oc5ccccc5c4c3)nc(-c3cc4ccccc4cc3-c3cccc4c3ccc3c5ccccc5ccc43)n2)cc1 ; c1ccc(-c2nc(-c3cccc4c3ccc3c5ccccc5ccc43)nc(-c3cccc4oc5ccccc5c34)n2)cc1]

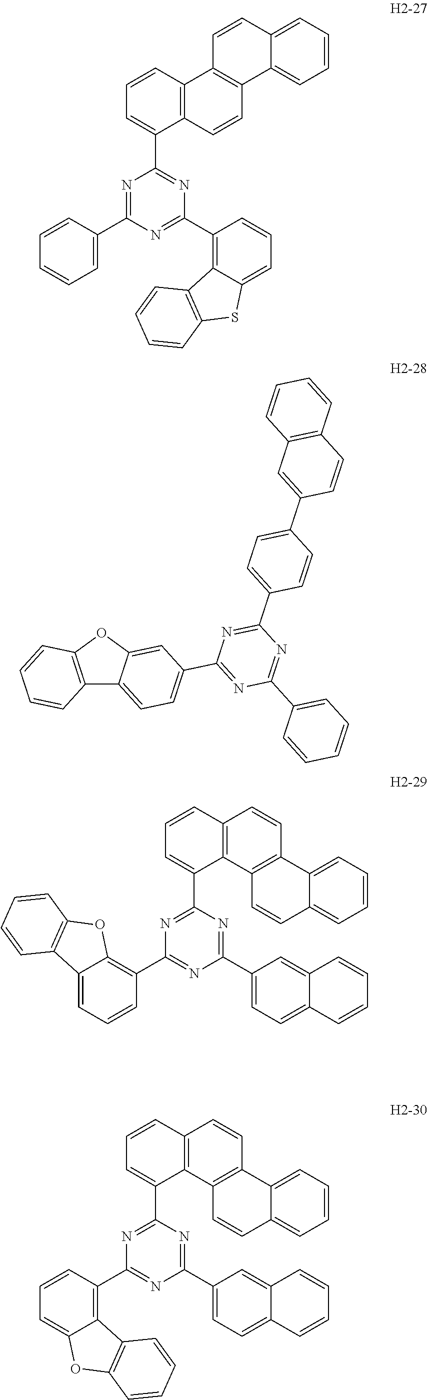 [SMILES: c1ccc(-c2nc(-c3cccc4c3ccc3c5ccccc5ccc43)nc(-c3cccc4sc5ccccc5c34)n2)cc1 ; c1ccc(-c2nc(-c3ccc(-c4ccc5ccccc5c4)cc3)nc(-c3ccc4c(c3)oc3ccccc34)n2)cc1 ; c1ccc2cc(-c3nc(-c4cccc5c4oc4ccccc45)nc(-c4cccc5ccc6c7ccccc7ccc6c45)n3)ccc2c1 ; c1ccc2cc(-c3nc(-c4cccc5ccc6c7ccccc7ccc6c45)nc(-c4cccc5oc6ccccc6c45)n3)ccc2c1]

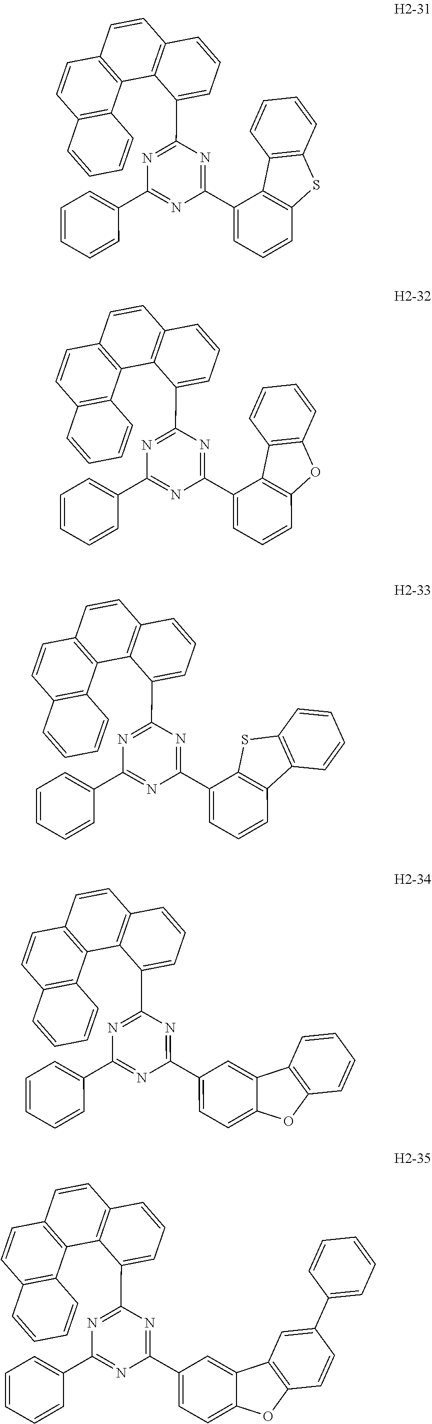 [SMILES: c1ccc(-c2nc(-c3cccc4sc5ccccc5c34)nc(-c3cccc4ccc5ccc6ccccc6c5c34)n2)cc1 ; c1ccc(-c2nc(-c3cccc4oc5ccccc5c34)nc(-c3cccc4ccc5ccc6ccccc6c5c34)n2)cc1 ; c1ccc(-c2nc(-c3cccc4c3sc3ccccc34)nc(-c3cccc4ccc5ccc6ccccc6c5c34)n2)cc1 ; c1ccc(-c2nc(-c3ccc4oc5ccccc5c4c3)nc(-c3cccc4ccc5ccc6ccccc6c5c34)n2)cc1 ; c1ccc(-c2ccc3oc4ccc(-c5nc(-c6ccccc6)nc(-c6cccc7ccc8ccc9ccccc9c8c67)n5)cc4c3c2)cc1]

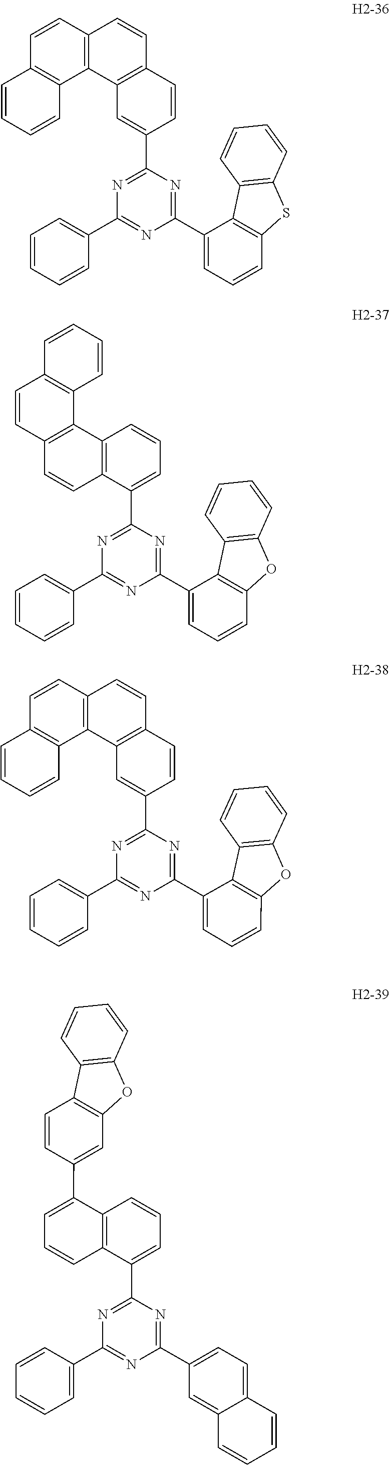

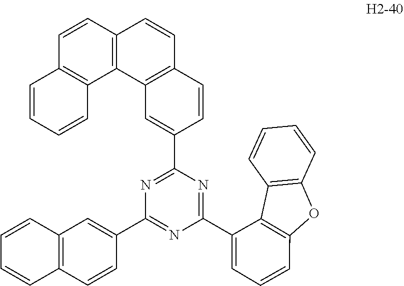 [SMILES: c1ccc2cc(-c3nc(-c4ccc5ccc6ccc7ccccc7c6c5c4)nc(-c4cccc5oc6ccccc6c45)n3)ccc2c1]

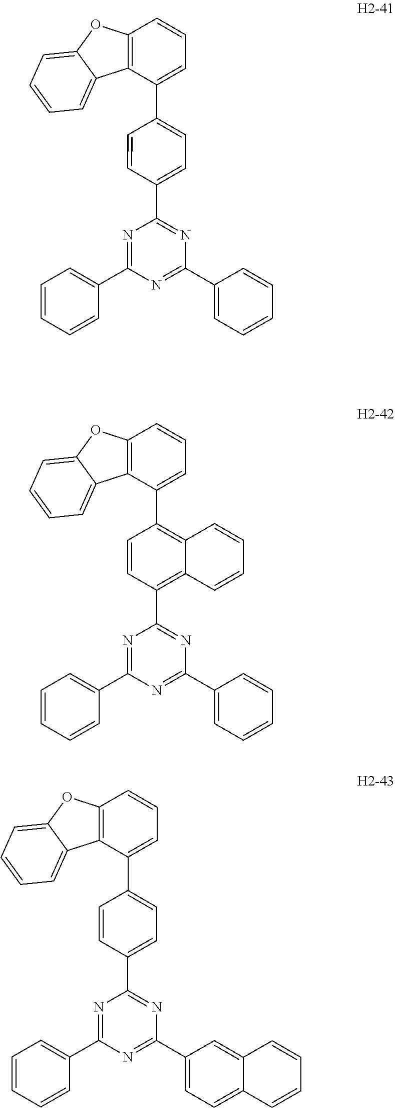 [SMILES: c1ccc(-c2nc(-c3ccccc3)nc(-c3ccc(-c4cccc5oc6ccccc6c45)cc3)n2)cc1 ; c1ccc(-c2nc(-c3ccccc3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccccc4c3)n2)cc1]

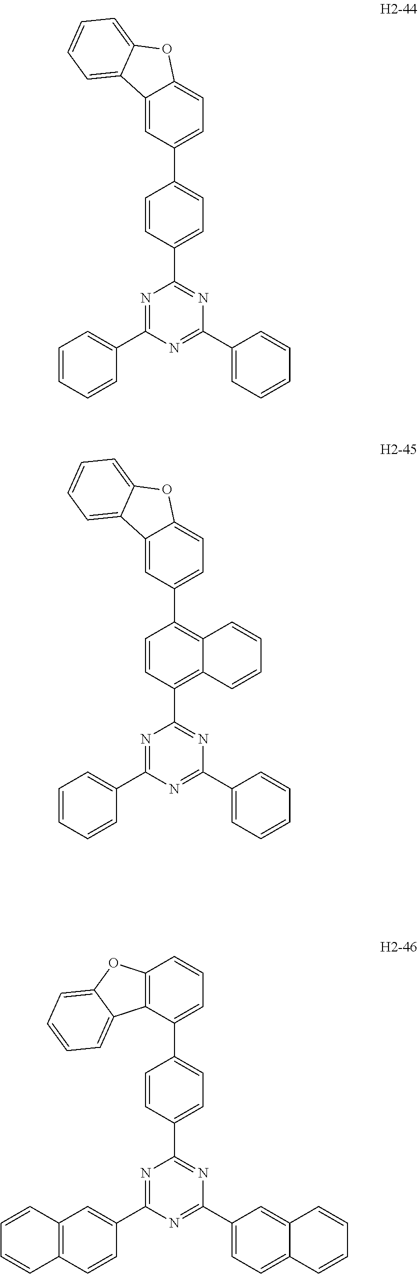 [SMILES: c1ccc(-c2nc(-c3ccccc3)nc(-c3ccc(-c4ccc5oc6ccccc6c5c4)cc3)n2)cc1 ; c1ccc(-c2nc(-c3ccccc3)nc(-c3ccc(-c4ccc5oc6ccccc6c5c4)c4ccccc34)n2)cc1 ; c1ccc2cc(-c3nc(-c4ccc(-c5cccc6oc7ccccc7c56)cc4)nc(-c4ccc5ccccc5c4)n3)ccc2c1]

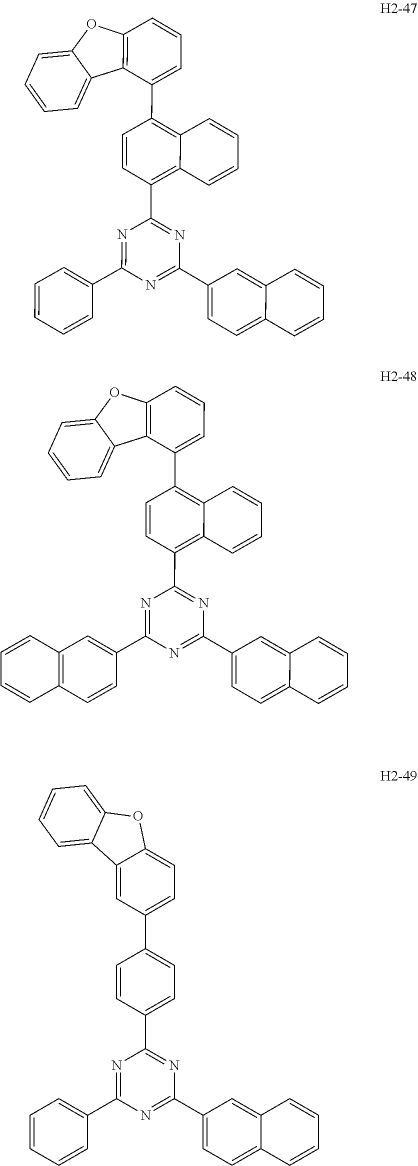 [SMILES: c1ccc(-c2nc(-c3ccc4ccccc4c3)nc(-c3ccc(-c4cccc5oc6ccccc6c45)c4ccccc34)n2)cc1 ; c1ccc2cc(-c3nc(-c4ccc5ccccc5c4)nc(-c4ccc(-c5cccc6oc7ccccc7c56)c5ccccc45)n3)ccc2c1 ; c1ccc(-c2nc(-c3ccc(-c4ccc5oc6ccccc6c5c4)cc3)nc(-c3ccc4ccccc4c3)n2)cc1]

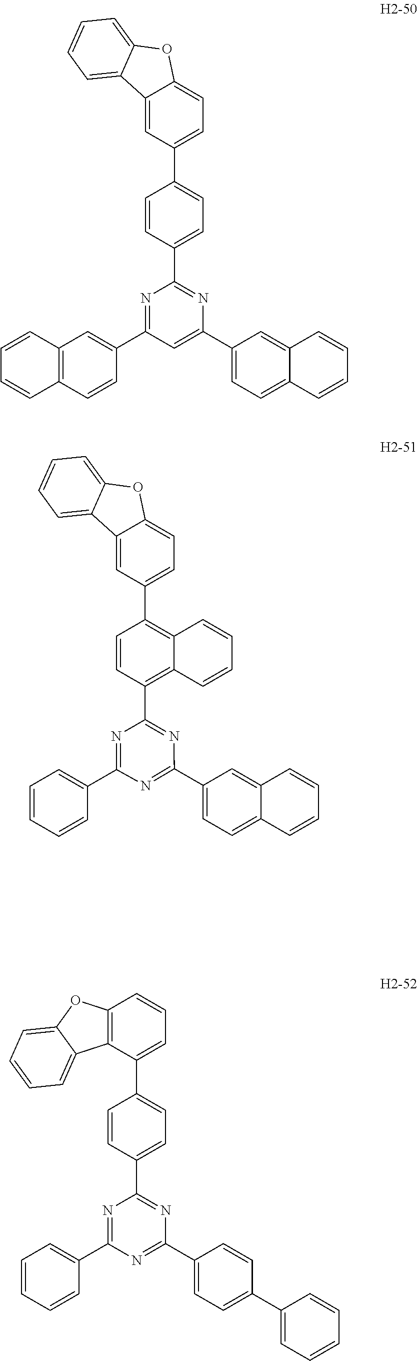

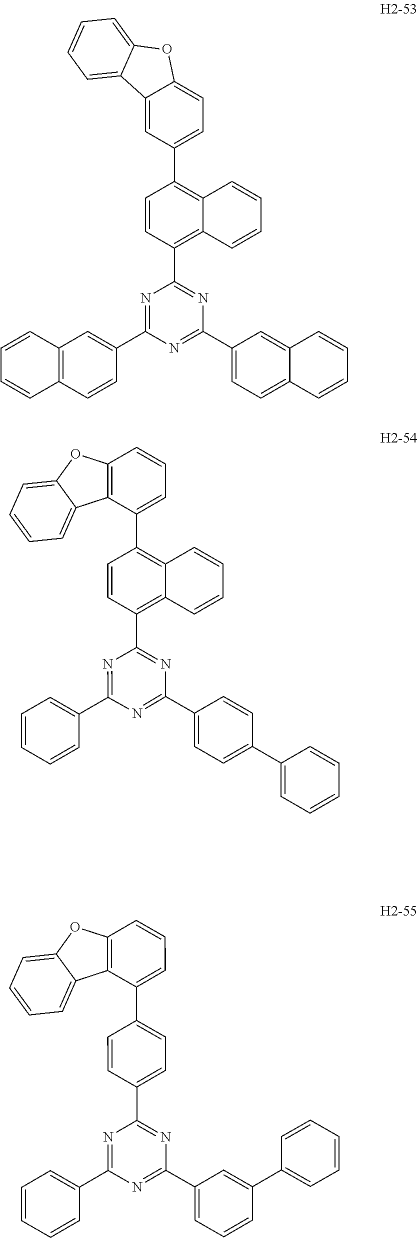

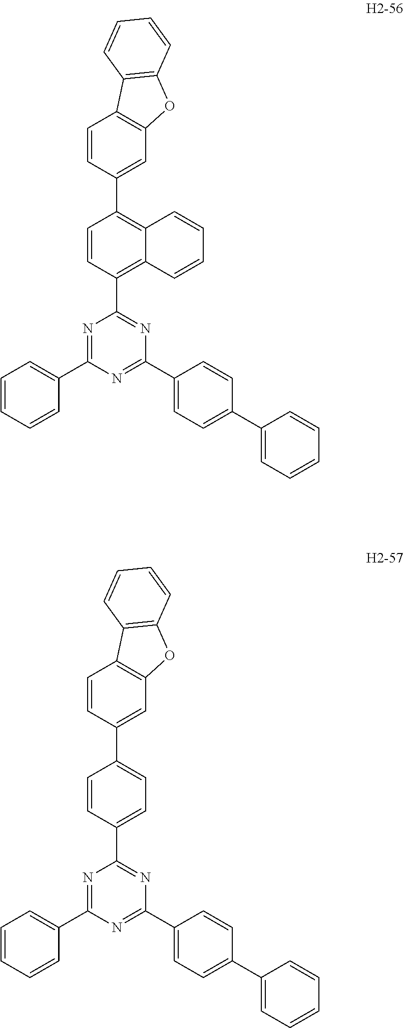 [SMILES: c1ccc(-c2ccc(-c3nc(-c4ccccc4)nc(-c4ccc(-c5ccc6c(c5)oc5ccccc56)c5ccccc45)n3)cc2)cc1 ; c1ccc(-c2ccc(-c3nc(-c4ccccc4)nc(-c4ccc(-c5ccc6c(c5)oc5ccccc56)cc4)n3)cc2)cc1]

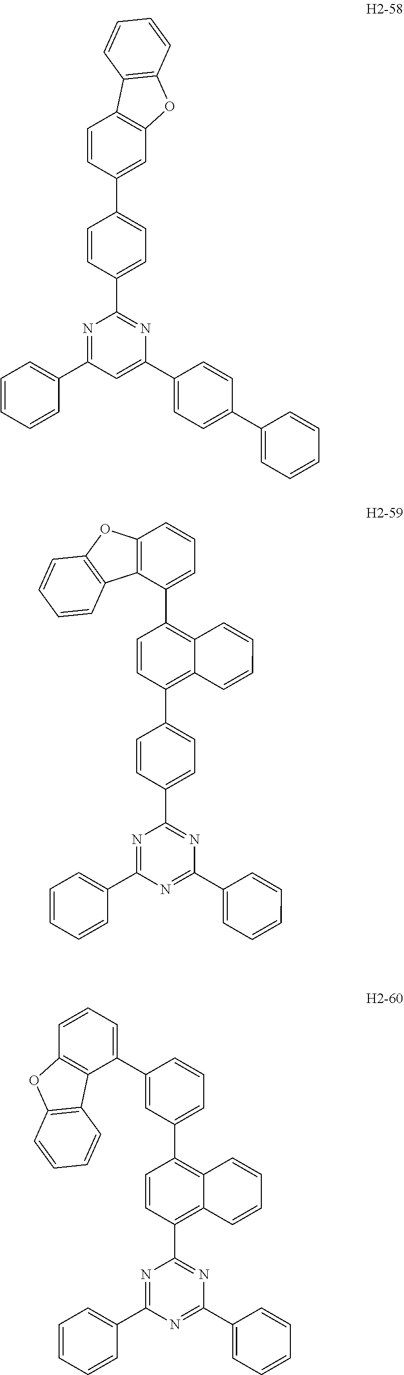 [SMILES: c1ccc(-c2ccc(-c3cc(-c4ccccc4)nc(-c4ccc(-c5ccc6c(c5)oc5ccccc56)cc4)n3)cc2)cc1 ; c1ccc(-c2nc(-c3ccccc3)nc(-c3ccc(-c4ccc(-c5cccc6oc7ccccc7c56)c5ccccc45)cc3)n2)cc1 ; c1ccc(-c2nc(-c3ccccc3)nc(-c3ccc(-c4cccc(-c5cccc6oc7ccccc7c56)c4)c4ccccc34)n2)cc1]

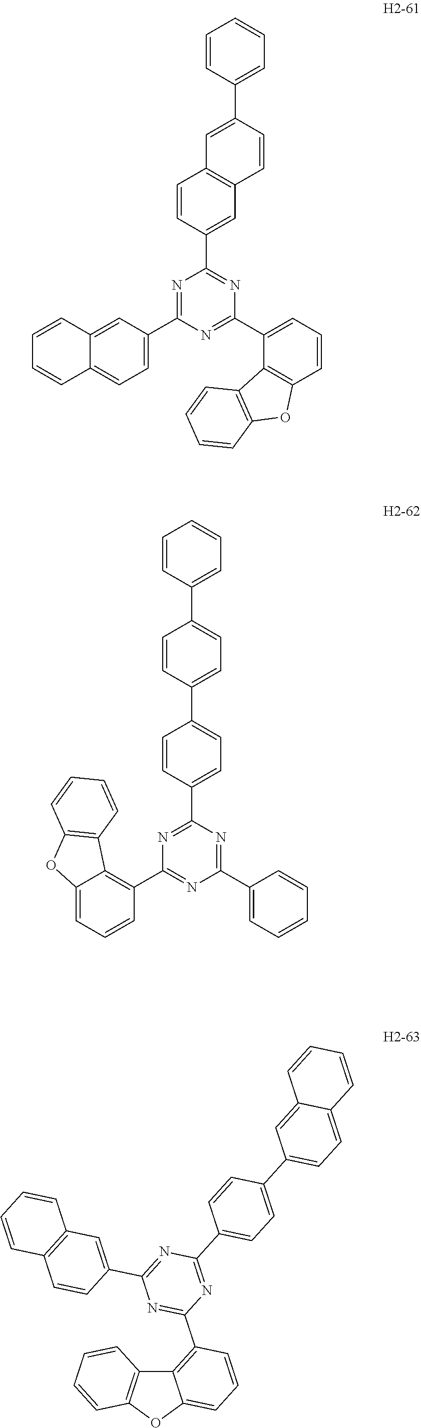 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccccc5)nc(-c5cccc6oc7ccccc7c56)n4)cc3)cc2)cc1 ; c1ccc2cc(-c3ccc(-c4nc(-c5ccc6ccccc6c5)nc(-c5cccc6oc7ccccc7c56)n4)cc3)ccc2c1]

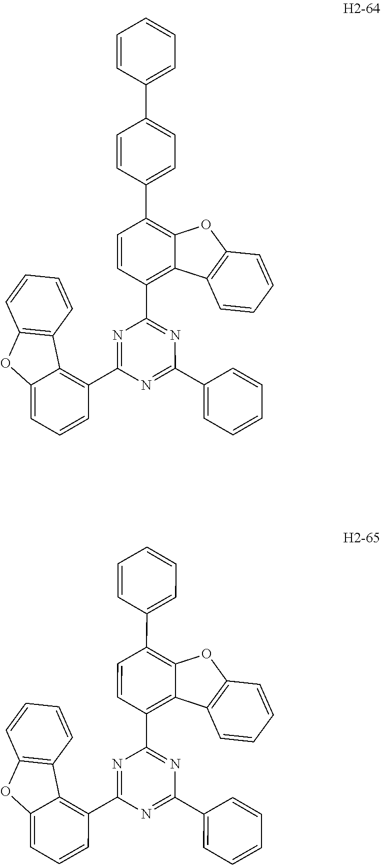 [SMILES: c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2nc(-c3cccc4oc5ccccc5c34)nc(-c3ccc(-c4ccccc4)c4oc5ccccc5c34)n2)cc1]

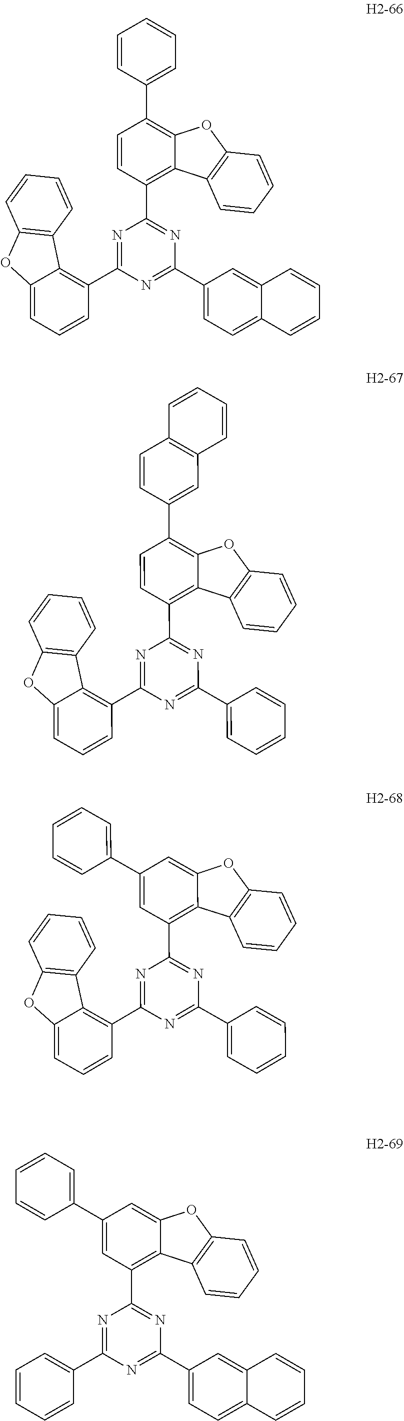 [SMILES: c1ccc(-c2ccc(-c3nc(-c4ccc5ccccc5c4)nc(-c4cccc5oc6ccccc6c45)n3)c3c2oc2ccccc23)cc1 ; c1ccc(-c2nc(-c3cccc4oc5ccccc5c34)nc(-c3ccc(-c4ccc5ccccc5c4)c4oc5ccccc5c34)n2)cc1 ; c1ccc(-c2cc(-c3nc(-c4ccccc4)nc(-c4cccc5oc6ccccc6c45)n3)c3c(c2)oc2ccccc23)cc1 ; c1ccc(-c2cc(-c3nc(-c4ccccc4)nc(-c4ccc5ccccc5c4)n3)c3c(c2)oc2ccccc23)cc1]

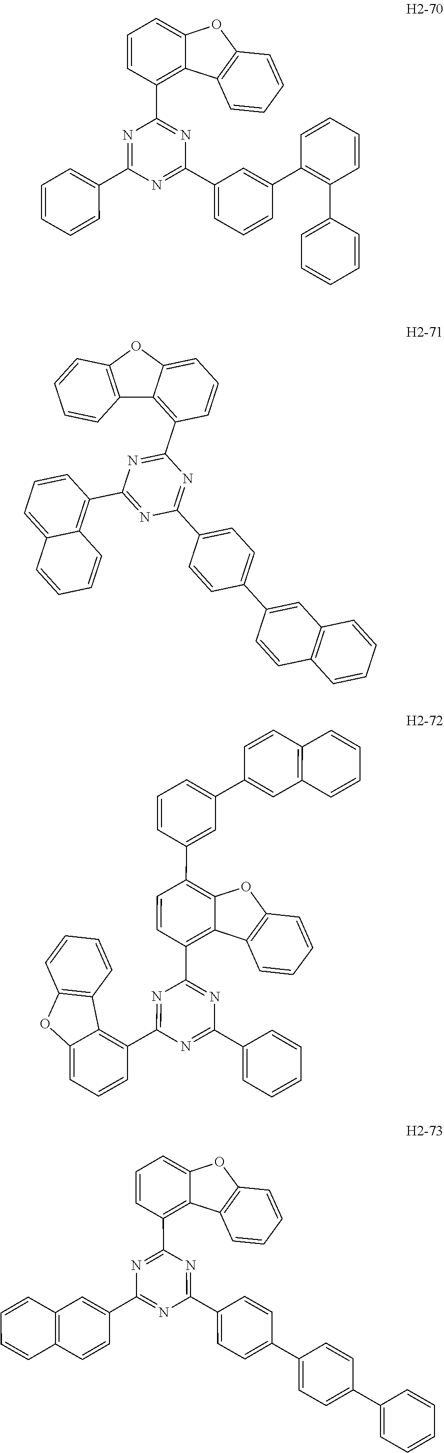 [SMILES: c1ccc(-c2nc(-c3cccc(-c4ccccc4-c4ccccc4)c3)nc(-c3cccc4oc5ccccc5c34)n2)cc1 ; c1ccc2cc(-c3ccc(-c4nc(-c5cccc6ccccc56)nc(-c5cccc6oc7ccccc7c56)n4)cc3)ccc2c1 ; c1ccc(-c2nc(-c3cccc4oc5ccccc5c34)nc(-c3ccc(-c4cccc(-c5ccc6ccccc6c5)c4)c4oc5ccccc5c34)n2)cc1 ; c1ccc(-c2ccc(-c3ccc(-c4nc(-c5ccc6ccccc6c5)nc(-c5cccc6oc7ccccc7c56)n4)cc3)cc2)cc1]

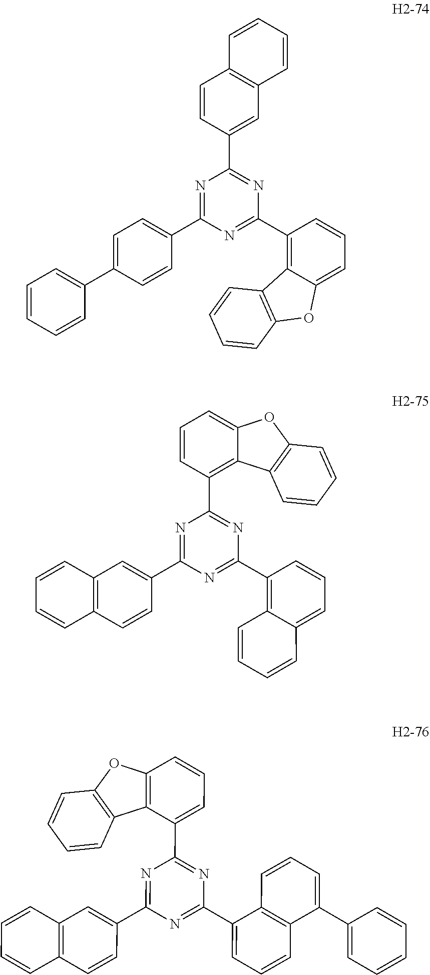 [SMILES: c1ccc(-c2ccc(-c3nc(-c4ccc5ccccc5c4)nc(-c4cccc5oc6ccccc6c45)n3)cc2)cc1 ; c1ccc2cc(-c3nc(-c4cccc5ccccc45)nc(-c4cccc5oc6ccccc6c45)n3)ccc2c1 ; c1ccc(-c2cccc3c(-c4nc(-c5ccc6ccccc6c5)nc(-c5cccc6oc7ccccc7c56)n4)cccc23)cc1]

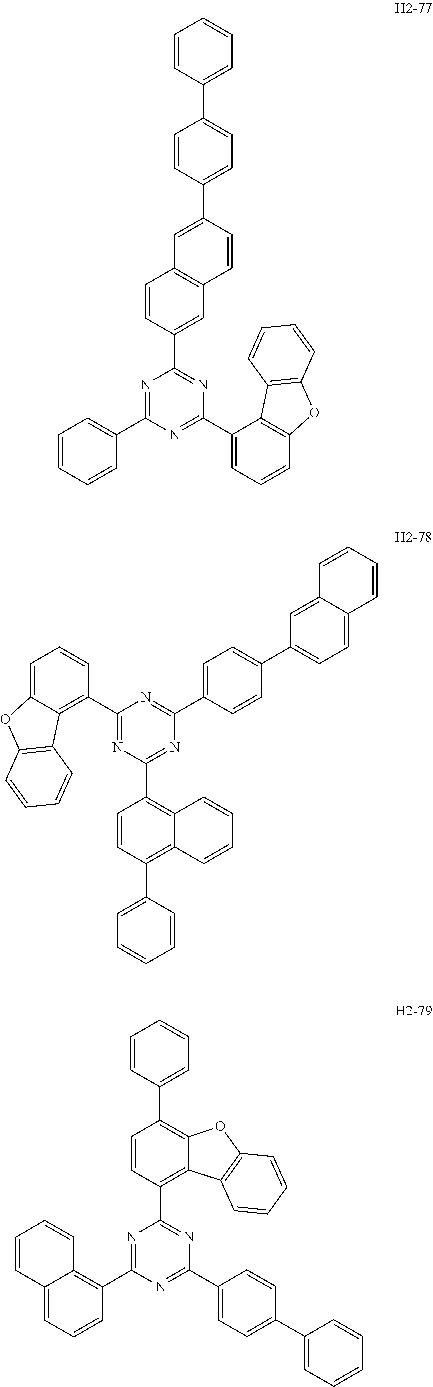

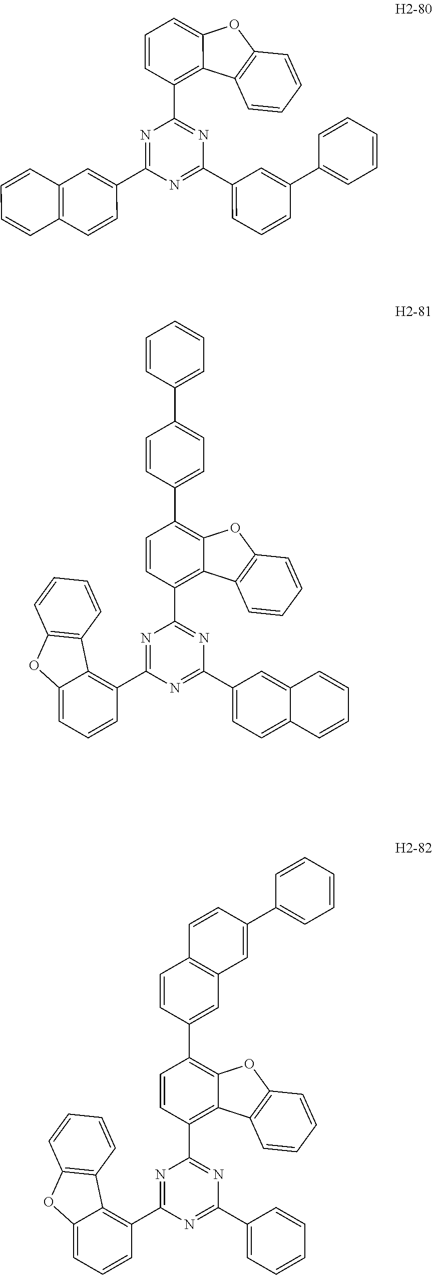 [SMILES: c1ccc(-c2cccc(-c3nc(-c4ccc5ccccc5c4)nc(-c4cccc5oc6ccccc6c45)n3)c2)cc1 ; c1ccc(-c2ccc(-c3ccc(-c4nc(-c5ccc6ccccc6c5)nc(-c5cccc6oc7ccccc7c56)n4)c4c3oc3ccccc34)cc2)cc1 ; c1ccc(-c2ccc3ccc(-c4ccc(-c5nc(-c6ccccc6)nc(-c6cccc7oc8ccccc8c67)n5)c5c4oc4ccccc45)cc3c2)cc1]

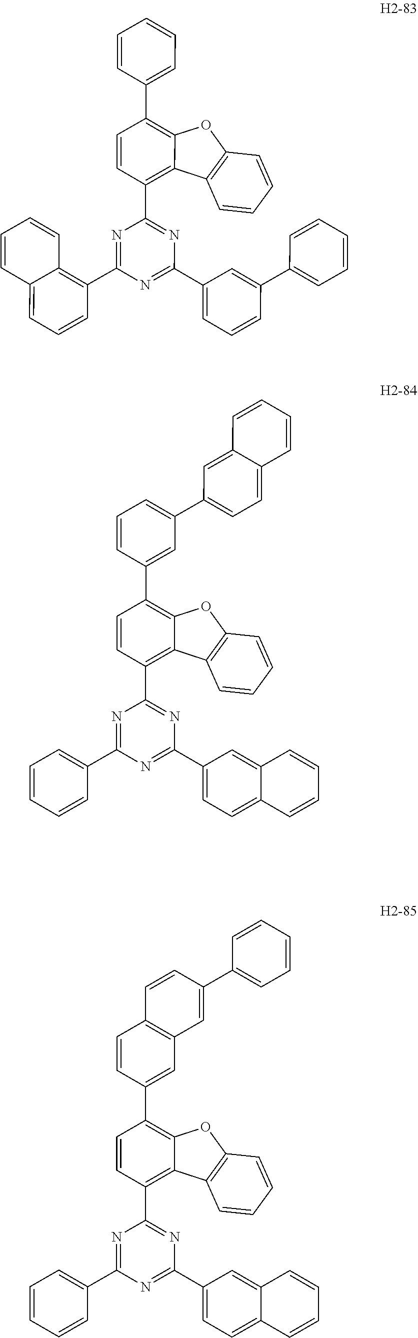 [SMILES: c1ccc(-c2cccc(-c3nc(-c4cccc5ccccc45)nc(-c4ccc(-c5ccccc5)c5oc6ccccc6c45)n3)c2)cc1 ; c1ccc(-c2nc(-c3ccc4ccccc4c3)nc(-c3ccc(-c4cccc(-c5ccc6ccccc6c5)c4)c4oc5ccccc5c34)n2)cc1 ; c1ccc(-c2ccc3ccc(-c4ccc(-c5nc(-c6ccccc6)nc(-c6ccc7ccccc7c6)n5)c5c4oc4ccccc45)cc3c2)cc1]

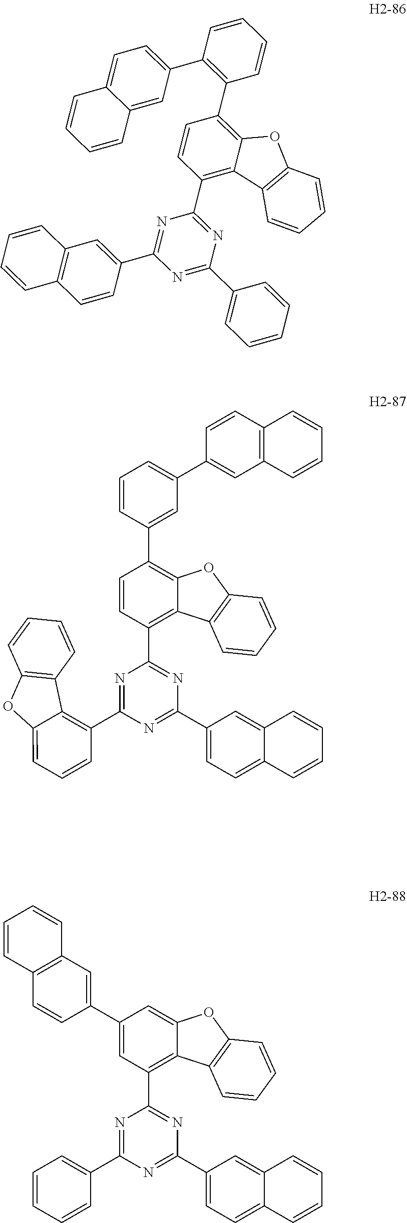 [SMILES: c1ccc(-c2nc(-c3ccc4ccccc4c3)nc(-c3ccc(-c4ccccc4-c4ccc5ccccc5c4)c4oc5ccccc5c34)n2)cc1 ; c1cc(-c2ccc3ccccc3c2)cc(-c2ccc(-c3nc(-c4ccc5ccccc5c4)nc(-c4cccc5oc6ccccc6c45)n3)c3c2oc2ccccc23)c1 ; c1ccc(-c2nc(-c3ccc4ccccc4c3)nc(-c3cc(-c4ccc5ccccc5c4)cc4oc5ccccc5c34)n2)cc1]

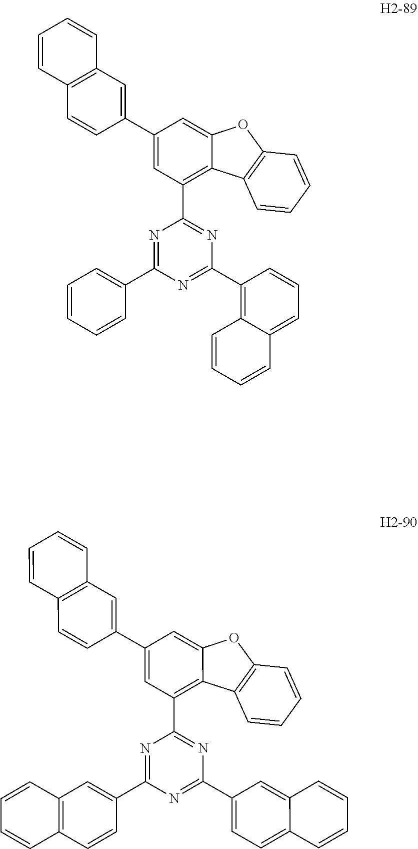 [SMILES: c1ccc(-c2nc(-c3cccc4ccccc34)nc(-c3cc(-c4ccc5ccccc5c4)cc4oc5ccccc5c34)n2)cc1 ; c1ccc2cc(-c3cc(-c4nc(-c5ccc6ccccc6c5)nc(-c5ccc6ccccc6c5)n4)c4c(c3)oc3ccccc34)ccc2c1]

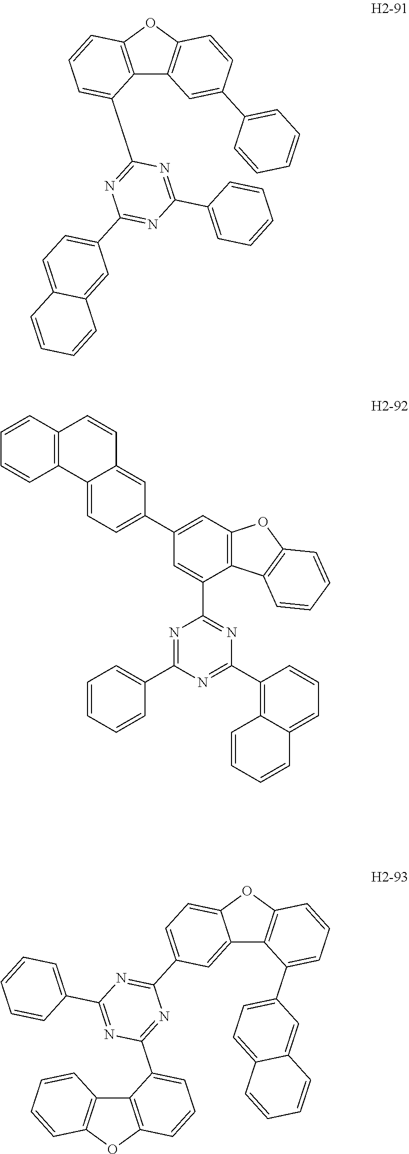

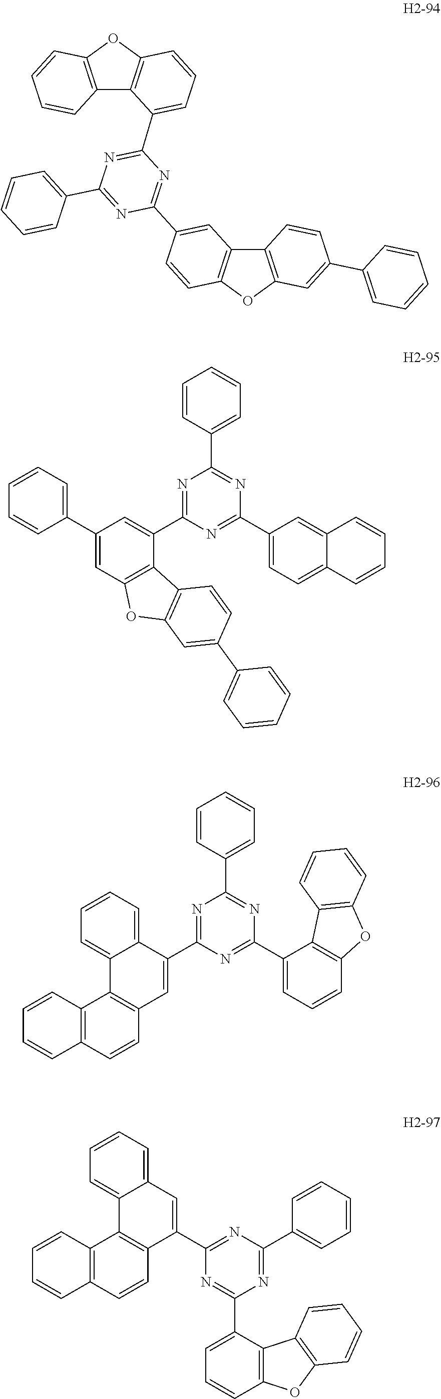 [SMILES: c1ccc(-c2ccc3c(c2)oc2ccc(-c4nc(-c5ccccc5)nc(-c5cccc6oc7ccccc7c56)n4)cc23)cc1 ; c1ccc(-c2ccc3c(c2)oc2cc(-c4ccccc4)cc(-c4nc(-c5ccccc5)nc(-c5ccc6ccccc6c5)n4)c23)cc1 ; c1ccc(-c2nc(-c3cc4ccc5ccccc5c4c4ccccc34)nc(-c3cccc4oc5ccccc5c34)n2)cc1 ; c1ccc(-c2nc(-c3cc4ccccc4c4c3ccc3ccccc34)nc(-c3cccc4oc5ccccc5c34)n2)cc1]

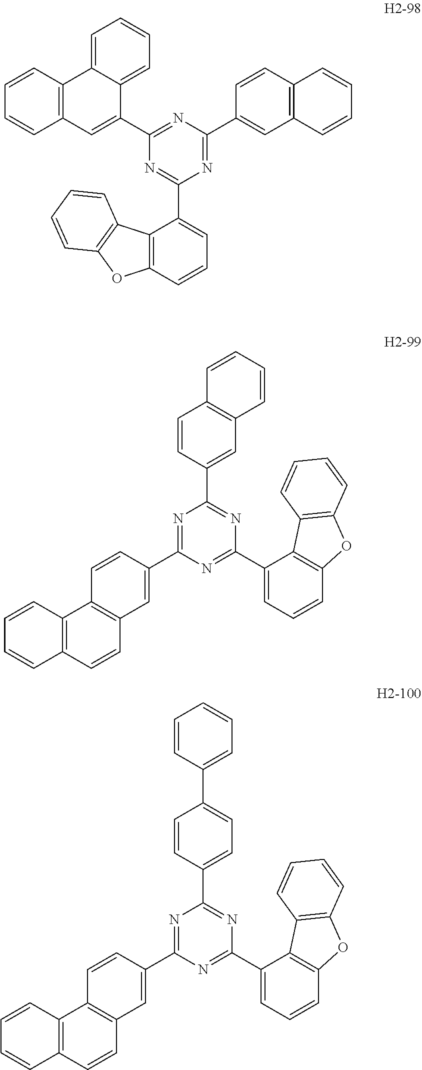 [SMILES: c1ccc2cc(-c3nc(-c4cc5ccccc5c5ccccc45)nc(-c4cccc5oc6ccccc6c45)n3)ccc2c1 ; c1ccc2cc(-c3nc(-c4ccc5c(ccc6ccccc65)c4)nc(-c4cccc5oc6ccccc6c45)n3)ccc2c1 ; c1ccc(-c2ccc(-c3nc(-c4ccc5c(ccc6ccccc65)c4)nc(-c4cccc5oc6ccccc6c45)n3)cc2)cc1]

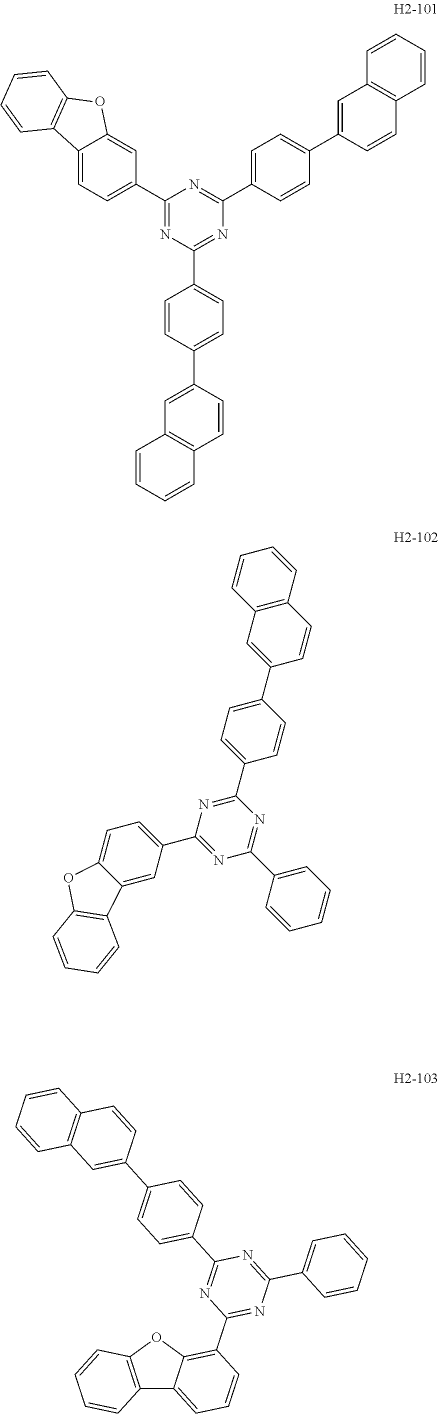 [SMILES: c1ccc2cc(-c3ccc(-c4nc(-c5ccc(-c6ccc7ccccc7c6)cc5)nc(-c5ccc6c(c5)oc5ccccc56)n4)cc3)ccc2c1 ; c1ccc(-c2nc(-c3ccc(-c4ccc5ccccc5c4)cc3)nc(-c3ccc4oc5ccccc5c4c3)n2)cc1 ; c1ccc(-c2nc(-c3ccc(-c4ccc5ccccc5c4)cc3)nc(-c3cccc4c3oc3ccccc34)n2)cc1]

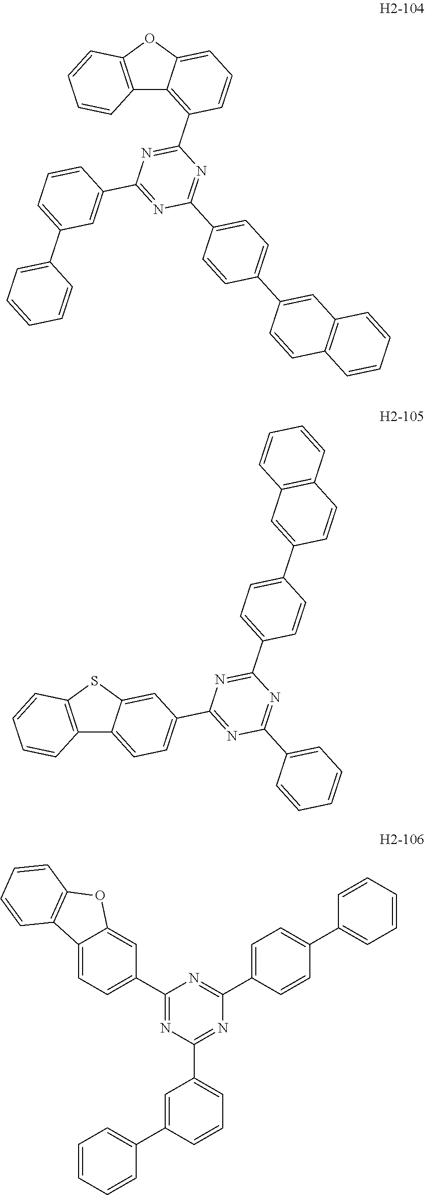 [SMILES: c1ccc(-c2cccc(-c3nc(-c4ccc(-c5ccc6ccccc6c5)cc4)nc(-c4cccc5oc6ccccc6c45)n3)c2)cc1 ; c1ccc(-c2nc(-c3ccc(-c4ccc5ccccc5c4)cc3)nc(-c3ccc4c(c3)sc3ccccc34)n2)cc1 ; c1ccc(-c2ccc(-c3nc(-c4cccc(-c5ccccc5)c4)nc(-c4ccc5c(c4)oc4ccccc45)n3)cc2)cc1]

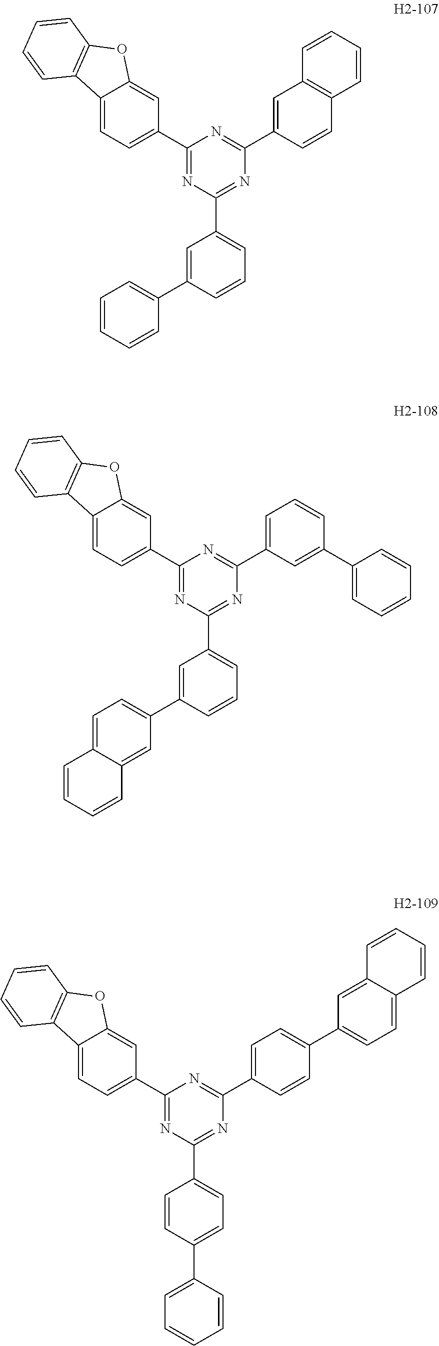 [SMILES: c1ccc(-c2cccc(-c3nc(-c4ccc5ccccc5c4)nc(-c4ccc5c(c4)oc4ccccc45)n3)c2)cc1 ; c1ccc(-c2cccc(-c3nc(-c4cccc(-c5ccc6ccccc6c5)c4)nc(-c4ccc5c(c4)oc4ccccc45)n3)c2)cc1 ; c1ccc(-c2ccc(-c3nc(-c4ccc(-c5ccc6ccccc6c5)cc4)nc(-c4ccc5c(c4)oc4ccccc45)n3)cc2)cc1]

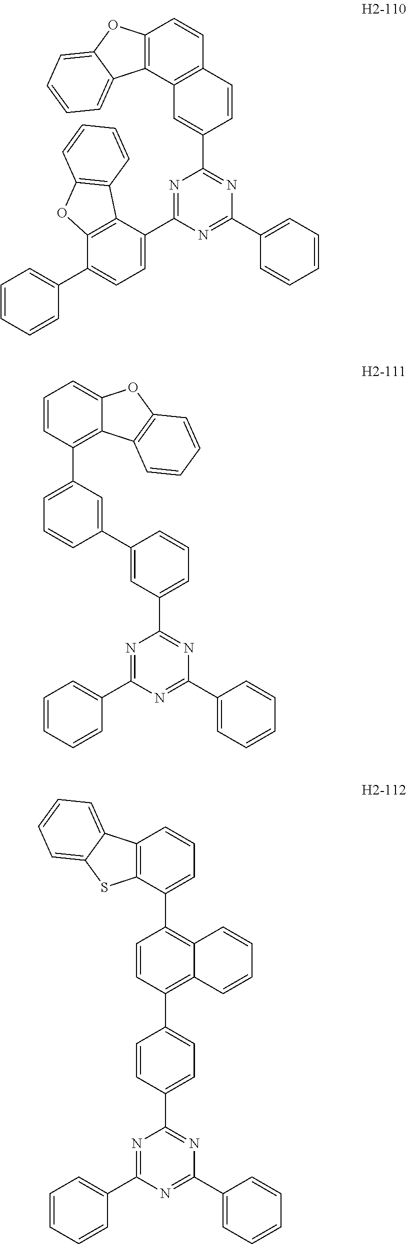 [SMILES: c1ccc(-c2nc(-c3ccc4ccc5oc6ccccc6c5c4c3)nc(-c3ccc(-c4ccccc4)c4oc5ccccc5c34)n2)cc1 ; c1ccc(-c2nc(-c3ccccc3)nc(-c3cccc(-c4cccc(-c5cccc6oc7ccccc7c56)c4)c3)n2)cc1 ; c1ccc(-c2nc(-c3ccccc3)nc(-c3ccc(-c4ccc(-c5cccc6c5sc5ccccc56)c5ccccc45)cc3)n2)cc1]

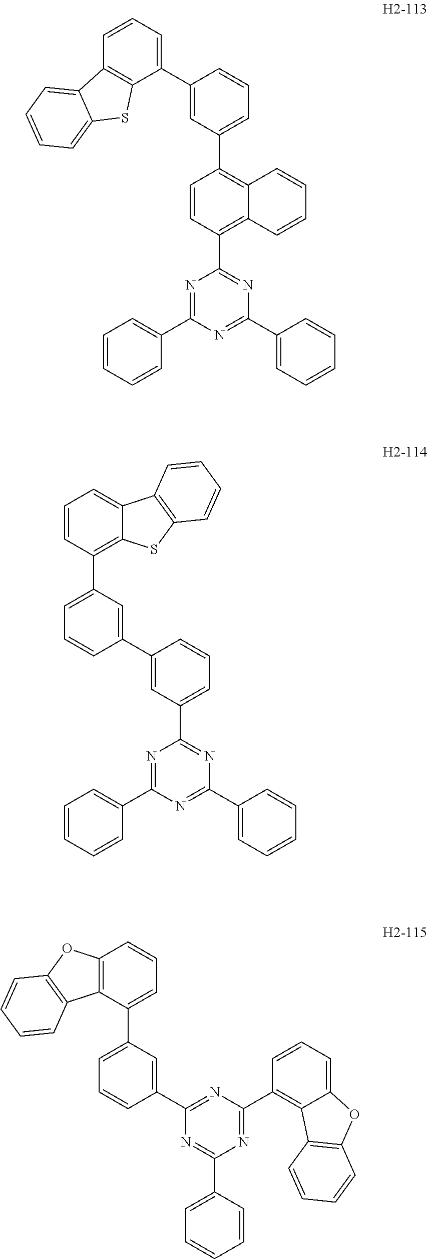 [SMILES: c1ccc(-c2nc(-c3ccccc3)nc(-c3ccc(-c4cccc(-c5cccc6c5sc5ccccc56)c4)c4ccccc34)n2)cc1 ; c1ccc(-c2nc(-c3ccccc3)nc(-c3cccc(-c4cccc(-c5cccc6c5sc5ccccc56)c4)c3)n2)cc1 ; c1ccc(-c2nc(-c3cccc(-c4cccc5oc6ccccc6c45)c3)nc(-c3cccc4oc5ccccc5c34)n2)cc1]

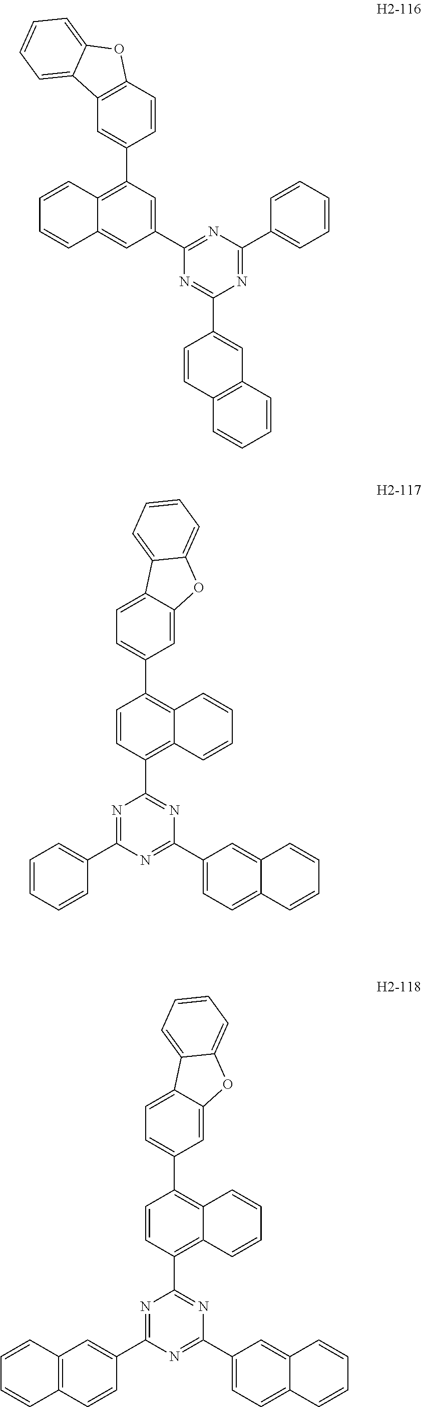 [SMILES: c1ccc(-c2nc(-c3ccc4ccccc4c3)nc(-c3cc(-c4ccc5oc6ccccc6c5c4)c4ccccc4c3)n2)cc1 ; c1ccc(-c2nc(-c3ccc4ccccc4c3)nc(-c3ccc(-c4ccc5c(c4)oc4ccccc45)c4ccccc34)n2)cc1 ; c1ccc2cc(-c3nc(-c4ccc5ccccc5c4)nc(-c4ccc(-c5ccc6c(c5)oc5ccccc56)c5ccccc45)n3)ccc2c1]

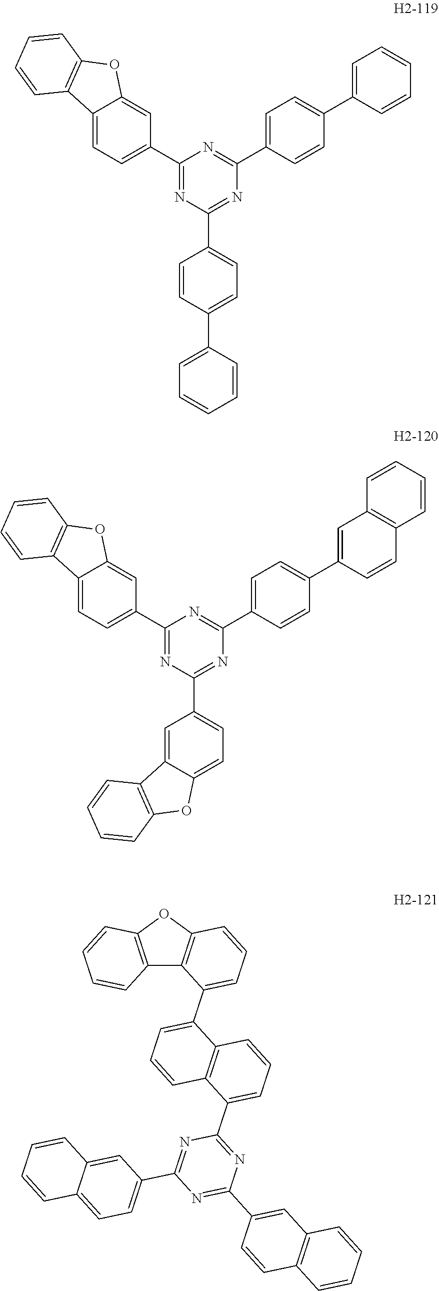 [SMILES: c1ccc(-c2ccc(-c3nc(-c4ccc(-c5ccccc5)cc4)nc(-c4ccc5c(c4)oc4ccccc45)n3)cc2)cc1 ; c1ccc2cc(-c3ccc(-c4nc(-c5ccc6c(c5)oc5ccccc56)nc(-c5ccc6oc7ccccc7c6c5)n4)cc3)ccc2c1 ; c1ccc2cc(-c3nc(-c4ccc5ccccc5c4)nc(-c4cccc5c(-c6cccc7oc8ccccc8c67)cccc45)n3)ccc2c1]

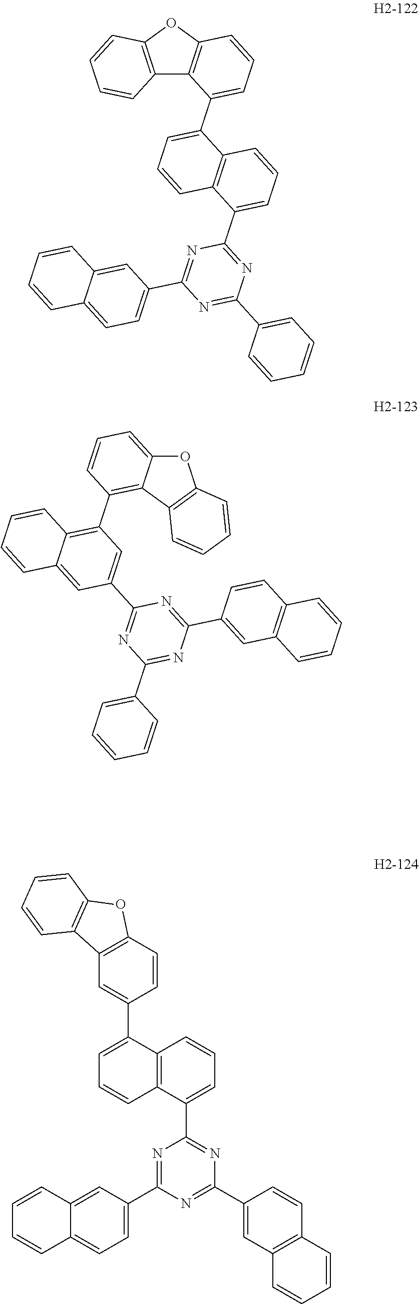 [SMILES: c1ccc(-c2nc(-c3ccc4ccccc4c3)nc(-c3cccc4c(-c5cccc6oc7ccccc7c56)cccc34)n2)cc1 ; c1ccc(-c2nc(-c3ccc4ccccc4c3)nc(-c3cc(-c4cccc5oc6ccccc6c45)c4ccccc4c3)n2)cc1 ; c1ccc2cc(-c3nc(-c4ccc5ccccc5c4)nc(-c4cccc5c(-c6ccc7oc8ccccc8c7c6)cccc45)n3)ccc2c1]

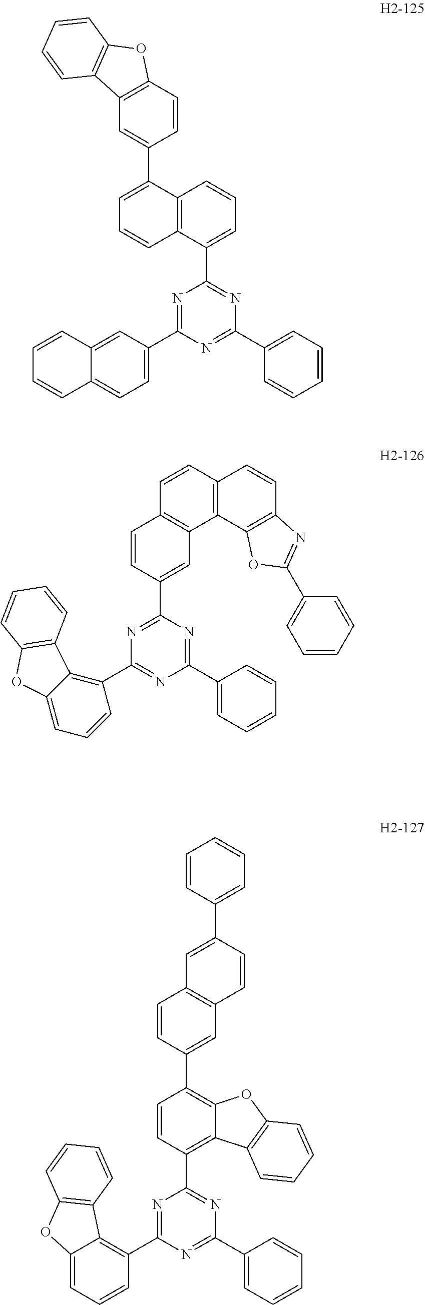 [SMILES: c1ccc(-c2nc(-c3ccc4ccccc4c3)nc(-c3cccc4c(-c5ccc6oc7ccccc7c6c5)cccc34)n2)cc1 ; c1ccc(-c2nc(-c3ccc4ccc5ccc6nc(-c7ccccc7)oc6c5c4c3)nc(-c3cccc4oc5ccccc5c34)n2)cc1 ; c1ccc(-c2ccc3cc(-c4ccc(-c5nc(-c6ccccc6)nc(-c6cccc7oc8ccccc8c67)n5)c5c4oc4ccccc45)ccc3c2)cc1]

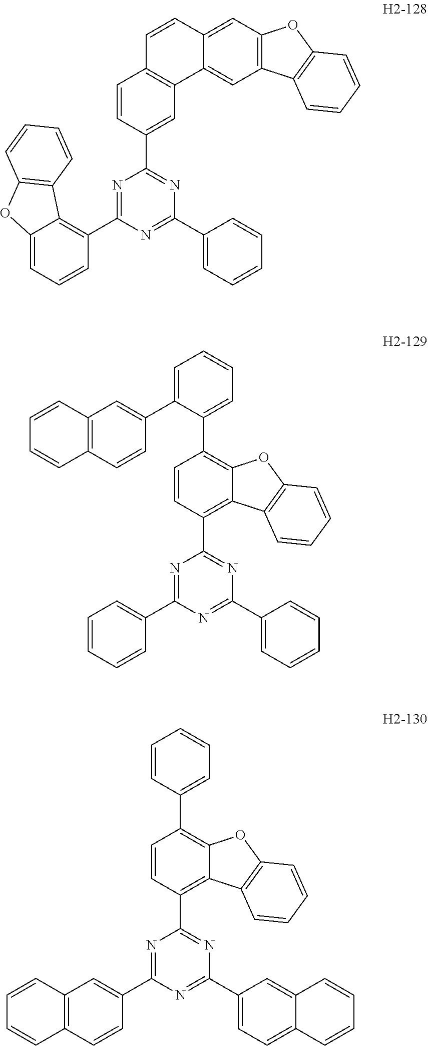 [SMILES: c1ccc(-c2nc(-c3ccc4ccc5cc6oc7ccccc7c6cc5c4c3)nc(-c3cccc4oc5ccccc5c34)n2)cc1 ; c1ccc(-c2nc(-c3ccccc3)nc(-c3ccc(-c4ccccc4-c4ccc5ccccc5c4)c4oc5ccccc5c34)n2)cc1 ; c1ccc(-c2ccc(-c3nc(-c4ccc5ccccc5c4)nc(-c4ccc5ccccc5c4)n3)c3c2oc2ccccc23)cc1]

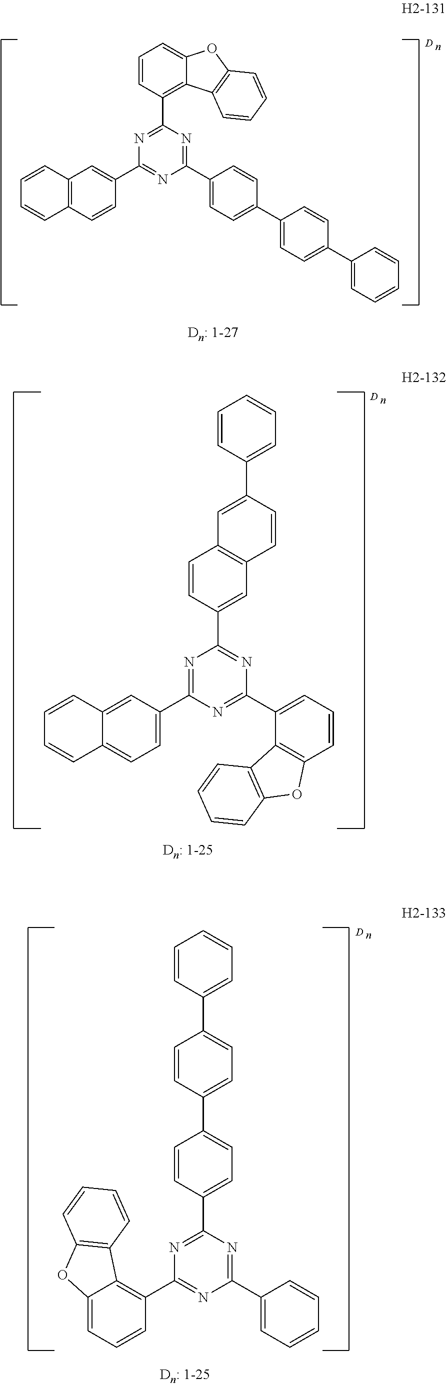

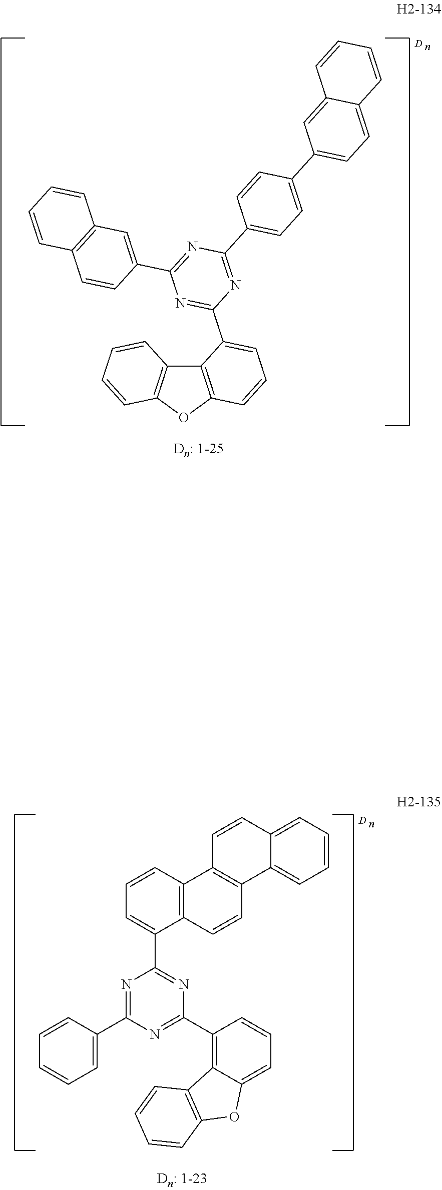

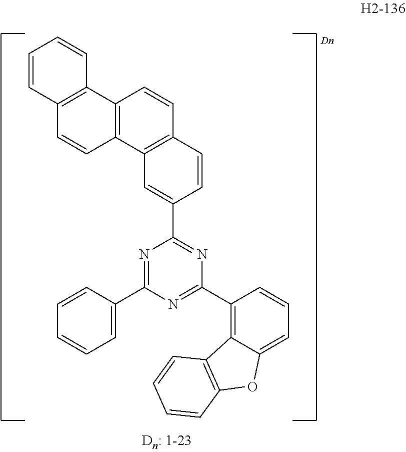

In each of Compounds H2-131 to H2-136, Dₙ means that 1 to 23, 1 to 25, or 1 to 27 hydrogens are replaced with deuterium.

In Formula 5, T₁ and T₂, each independently, represent —N═, —NR₄₀—, —O—, or —S—, with the proviso that any one of T₁ and T₂ represents —N═, and the other of T₁ and T₂ represents —NR₄₀—, —O—, or —S—. According to one embodiment of the present disclosure, any one of T₁ and T₂ represents —N═, and the other of T₁ and T₂ represents —O—, or —S—.

In Formula 5, R₃₁ represents a substituted or unsubstituted (C6-C30)aryl, or a substituted or unsubstituted (3- to 30-membered)heteroaryl. According to one embodiment of the present disclosure, R₃₁ represents a substituted or unsubstituted (C6-C25)aryl, or a substituted or unsubstituted (5- to 25-membered)heteroaryl. According to another embodiment of the present disclosure, R₃₁ represents a (C6-C18)aryl unsubstituted or substituted with deuterium, or a (5- to 20-membered)heteroaryl unsubstituted or substituted with deuterium. For example, R₃₁ may be a phenyl, a naphthyl, a biphenyl, a pyridyl, etc., which may be substituted with deuterium.

In Formula 5, R₃₂ to R₄₀, each independently, are represented by the following Formula 5-1, or represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di-(C6-C30)arylamino, a substituted or unsubstituted mono- or di-(3- to 30-membered)heteroarylamino, or a substituted or unsubstituted (C6-C30)aryl(3- to 30-membered)heteroarylamino; or may be linked to an adjacent substituent(s) to form a ring(s); with the proviso that at least one of R₃₂ to R₃₉ is represented by Formula 5-1. According to one embodiment of the present disclosure, R₃₂ to R₄₀, each independently, are represented by Formula 5-1, or represent hydrogen, deuterium, or a substituted or unsubstituted (C6-C25)aryl. According to another embodiment of the present disclosure, R₃₂ to R₄₀, each independently, are represented by Formula 5-1, or represent hydrogen, deuterium, or a (C6-C18)aryl unsubstituted or substituted with deuterium. For example, R₃₂ to R₄₀, each independently, may be represented by Formula 5-1, or represent hydrogen, deuterium, or a phenyl unsubstituted or substituted with deuterium.

In Formula 5-1, L₆ to L₈, each independently, represent a single bond, a substituted or unsubstituted (C6-C30)arylene, or a substituted or unsubstituted (3- to 30-membered)heteroarylene. According to one embodiment of the present disclosure, L₆ to L₈, each independently, represent a single bond, a substituted or unsubstituted (C6-C25)arylene, or a substituted or unsubstituted (5- to 25-membered)heteroarylene. According to another embodiment of the present disclosure, L₆ to L₈, each independently, represent a single bond, a substituted or unsubstituted (C6-C18)arylene, or a substituted or unsubstituted (5- to 13-membered)heteroarylene, which can be substituted with deuterium, a (C6-C30)aryl(s), etc. For example, L₆ to L₈, each independently, may be a single bond, a phenylene unsubstituted or substituted with a phenyl(s), a naphthylene unsubstituted or substituted with a phenyl(s), a biphenylene, a phenanthrenylene, a dibenzofuranylene, a dibenzothiophenylene, a carbazolylene, etc., which may be substituted with deuterium.

In Formula 5-1, Ar₅ and Ar₆, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted (C6-C30)aryl(3- to 30-membered)heteroarylamino, or a substituted or unsubstituted fused ring group of a (C3-C30)aliphatic ring(s) and a (C6-C30)aromatic ring(s), or may be linked to an adjacent substituent(s) to form a ring(s). According to one embodiment of the present disclosure, Ar₅ and Ar₆, each independently, represent a substituted or unsubstituted (C6-C25)aryl, a substituted or unsubstituted (5- to 25-membered)heteroaryl, a substituted or unsubstituted tri(C6-C25)arylsilyl, or a substituted or unsubstituted di(C6-C25)arylamino. According to another embodiment of the present disclosure, Ar₅ and Ar₆, each independently, represent a substituted or unsubstituted (C6-C25)aryl, a substituted or unsubstituted (6- to 17-membered)heteroaryl, a substituted or unsubstituted tri(C6-C18)arylsilyl, or a substituted or unsubstituted di(C6-C18)arylamino, which may be substituted with at least one of deuterium, a (C1-C30)alkyl(s), a (C6-C30)aryl(s), a (3- to 30-membered)heteroaryl(s), a di(C6-C30)arylamino(s), and a tri(C6-C30)arylsilyl(s). For example, Ar₅ and Ar₆, each independently, may be a phenyl, a naphthyl, a biphenyl, a phenylnaphthyl, a naphthylphenyl, a phenanthrenyl, a anthracenyl, a dimethylfluorenyl, a phenylfluorenyl, a diphenylfluorenyl, a dimethylbenzofluorenyl, a spirobifluorenyl, a fluoranthenyl, a quarterphenyl, a C22 aryl, a pyridyl, a benzoimidazolyl, a dibenzofuranyl, a dibenzothiophenyl, a carbazolyl, a dibenzoselenophenyl, a benzofuropyridyl, a naphthobenzofuranyl, a naphthobenzothiophenyl, a phenoxazinyl, a triphenylsilyl, or a diphenylamino, etc., which may be substituted with at least one of deuterium, a methyl(s), a tert-butyl(s), a phenyl(s), a naphthyl(s), a biphenyl(s), a pyridyl(s), a diphenylamino(s), and a triphenylsilyl(s).

The compound represented by Formula 5 may be at least one selected from the group consisting of the following compounds, but is not limited thereto.

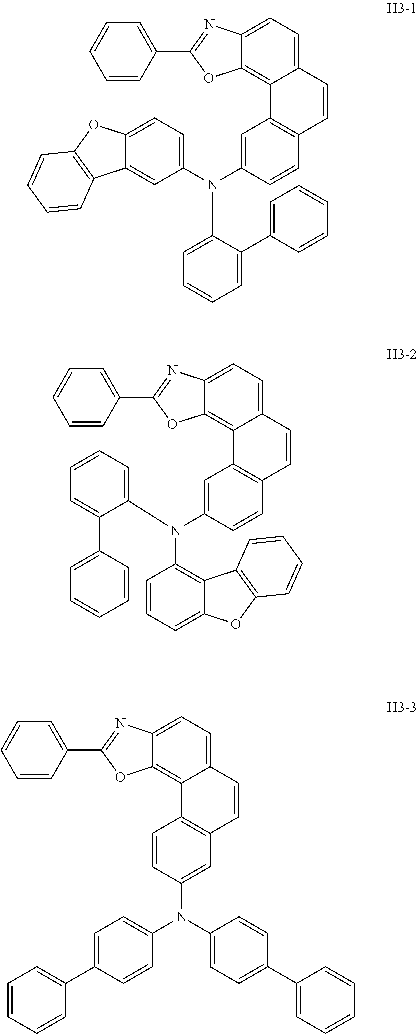 [SMILES: c1ccc(-c2nc3ccc4ccc5ccc(N(c6ccc7oc8ccccc8c7c6)c6ccccc6-c6ccccc6)cc5c4c3o2)cc1 ; c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1]

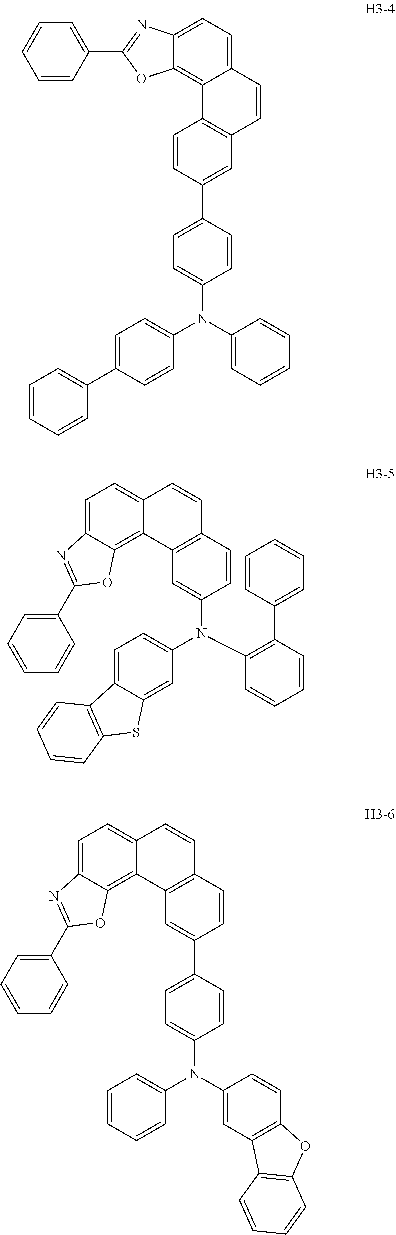 [SMILES: c1ccc(-c2ccc(N(c3ccccc3)c3ccc(-c4ccc5c(ccc6ccc7nc(-c8ccccc8)oc7c65)c4)cc3)cc2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1]

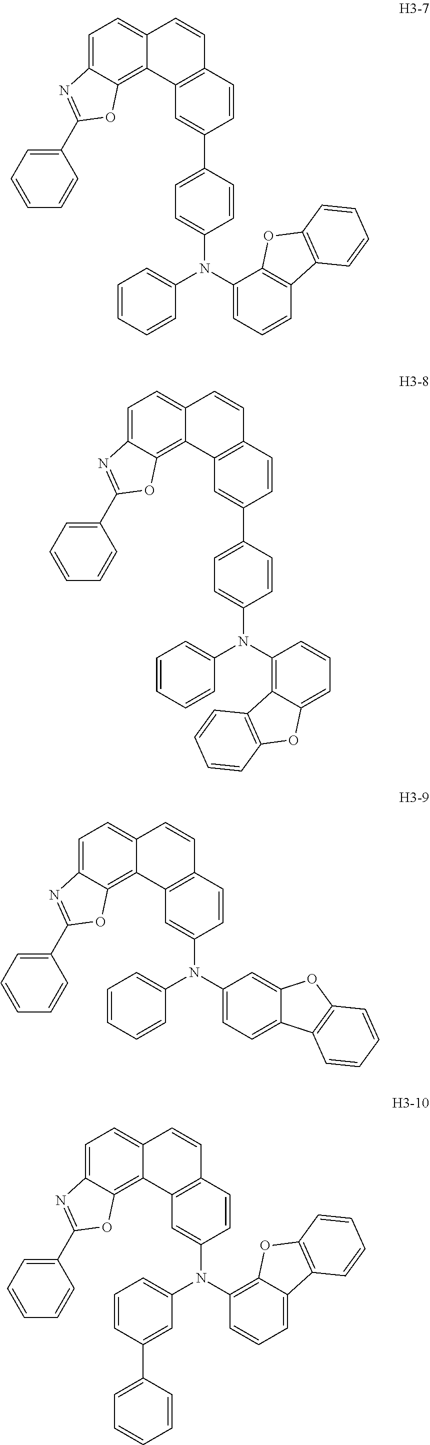 [SMILES: c1ccc(-c2nc3ccc4ccc5ccc(-c6ccc(N(c7ccccc7)c7cccc8c7oc7ccccc78)cc6)cc5c4c3o2)cc1 ; c1ccc(-c2nc3ccc4ccc5ccc(-c6ccc(N(c7ccccc7)c7cccc8oc9ccccc9c78)cc6)cc5c4c3o2)cc1 ; c1ccc(-c2nc3ccc4ccc5ccc(N(c6ccccc6)c6ccc7c(c6)oc6ccccc67)cc5c4c3o2)cc1 ; c1ccc(-c2cccc(N(c3ccc4ccc5ccc6nc(-c7ccccc7)oc6c5c4c3)c3cccc4c3oc3ccccc34)c2)cc1]

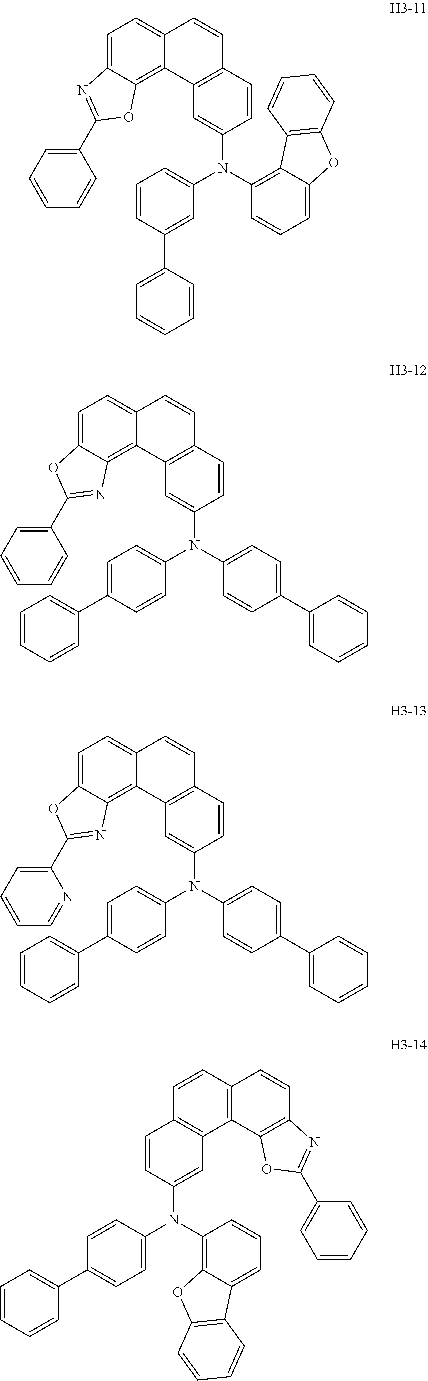 [SMILES: c1ccc(-c2cccc(N(c3ccc4ccc5ccc6nc(-c7ccccc7)oc6c5c4c3)c3cccc4oc5ccccc5c34)c2)cc1 ; c1ccc(-c2ccc(N(c3ccc(-c4ccccc4)cc3)c3ccc4ccc5ccc6oc(-c7ccccc7)nc6c5c4c3)cc2)cc1 ; c1ccc(-c2ccc(N(c3ccc(-c4ccccc4)cc3)c3ccc4ccc5ccc6oc(-c7ccccn7)nc6c5c4c3)cc2)cc1 ; c1ccc(-c2ccc(N(c3ccc4ccc5ccc6nc(-c7ccccc7)oc6c5c4c3)c3cccc4c3oc3ccccc34)cc2)cc1]

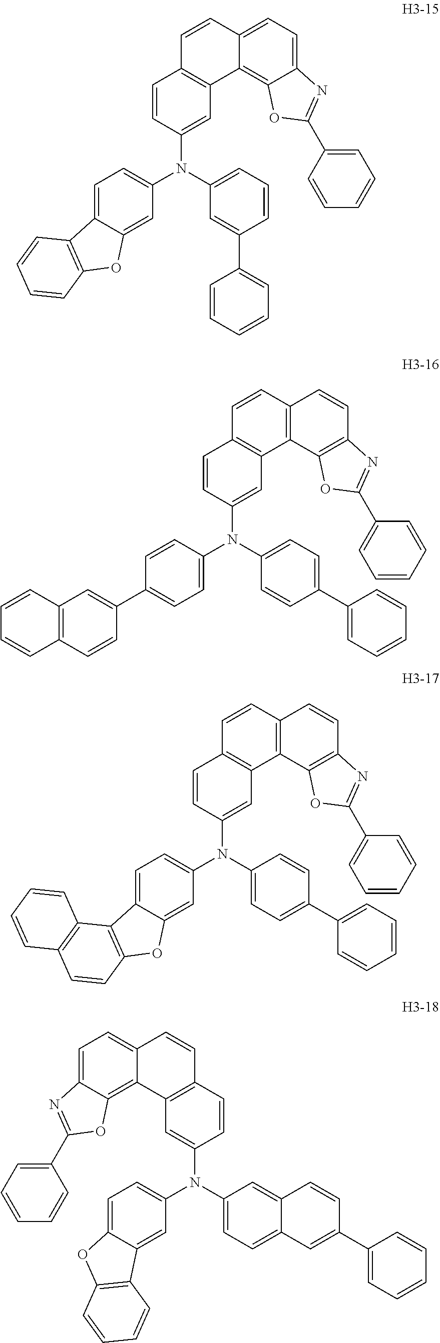 [SMILES: c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc(-c4ccc5ccccc5c4)cc3)c3ccc4ccc5ccc6nc(-c7ccccc7)oc6c5c4c3)cc2)cc1 ; c1ccc(-c2ccc(N(c3ccc4c(c3)oc3ccc5ccccc5c34)c3ccc4ccc5ccc6nc(-c7ccccc7)oc6c5c4c3)cc2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1]

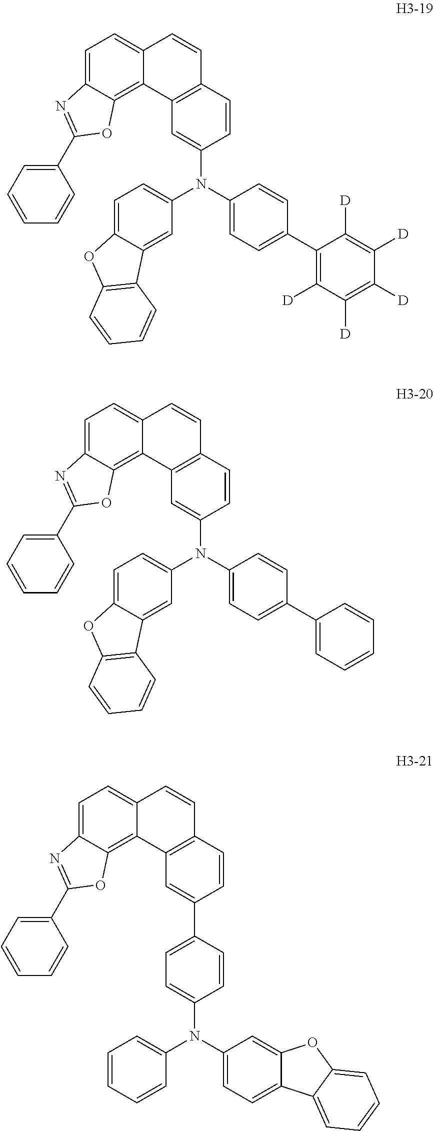 [SMILES: [2H]c1c([2H])c([2H])c(-c2ccc(N(c3ccc4oc5ccccc5c4c3)c3ccc4ccc5ccc6nc(-c7ccccc7)oc6c5c4c3)cc2)c([2H])c1[2H] ; c1ccc(-c2ccc(N(c3ccc4oc5ccccc5c4c3)c3ccc4ccc5ccc6nc(-c7ccccc7)oc6c5c4c3)cc2)cc1 ; c1ccc(-c2nc3ccc4ccc5ccc(-c6ccc(N(c7ccccc7)c7ccc8c(c7)oc7ccccc78)cc6)cc5c4c3o2)cc1]

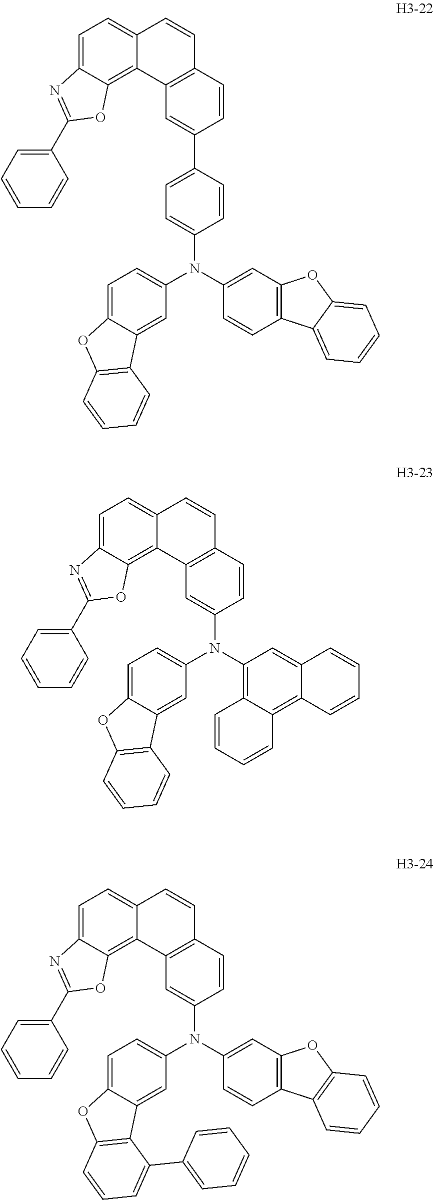 [SMILES: c1ccc(-c2nc3ccc4ccc5ccc(-c6ccc(N(c7ccc8c(c7)oc7ccccc78)c7ccc8oc9ccccc9c8c7)cc6)cc5c4c3o2)cc1 ; c1ccc(-c2nc3ccc4ccc5ccc(N(c6ccc7oc8ccccc8c7c6)c6cc7ccccc7c7ccccc67)cc5c4c3o2)cc1 ; c1ccc(-c2nc3ccc4ccc5ccc(N(c6ccc7c(c6)oc6ccccc67)c6ccc7oc8cccc(-c9ccccc9)c8c7c6)cc5c4c3o2)cc1]

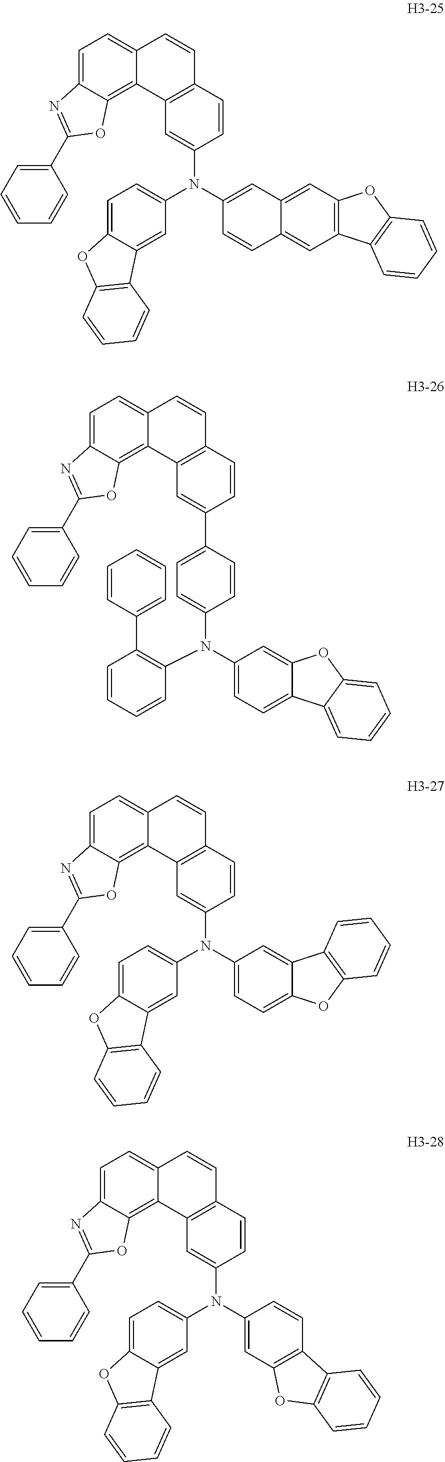 [SMILES: c1ccc(-c2nc3ccc4ccc5ccc(N(c6ccc7cc8c(cc7c6)oc6ccccc68)c6ccc7oc8ccccc8c7c6)cc5c4c3o2)cc1 ; c1ccc(-c2nc3ccc4ccc5ccc(-c6ccc(N(c7ccc8c(c7)oc7ccccc78)c7ccccc7-c7ccccc7)cc6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3o2)cc1]

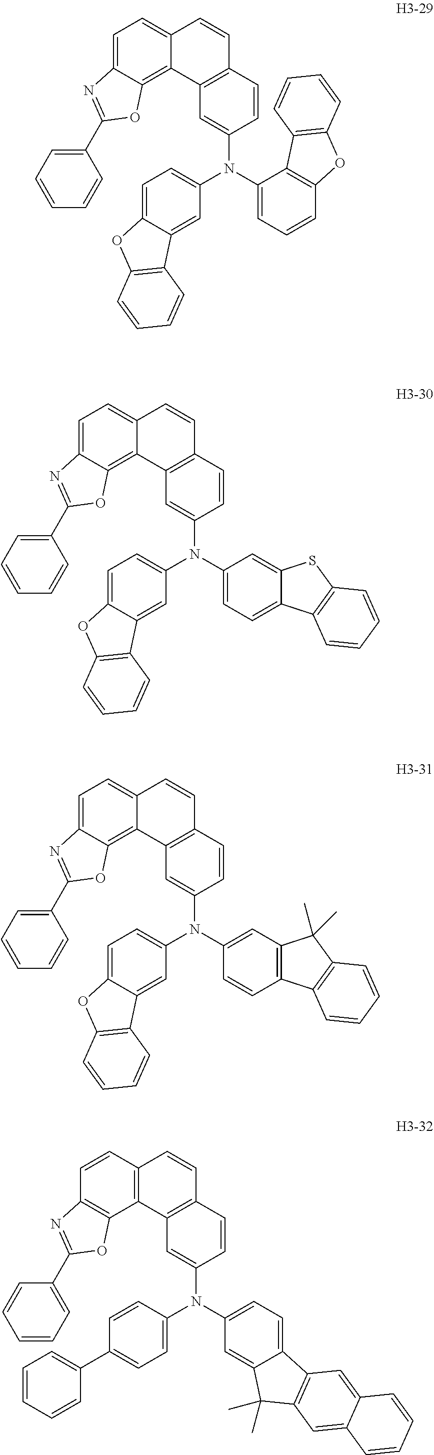 [SMILES: c1ccc(-c2nc3ccc4ccc5ccc(N(c6ccc7oc8ccccc8c7c6)c6cccc7oc8ccccc8c67)cc5c4c3o2)cc1 ; c1ccc(-c2nc3ccc4ccc5ccc(N(c6ccc7c(c6)sc6ccccc67)c6ccc7oc8ccccc8c7c6)cc5c4c3o2)cc1 ; CC1(C)c2ccccc2-c2ccc(N(c3ccc4oc5ccccc5c4c3)c3ccc4ccc5ccc6nc(-c7ccccc7)oc6c5c4c3)cc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21]

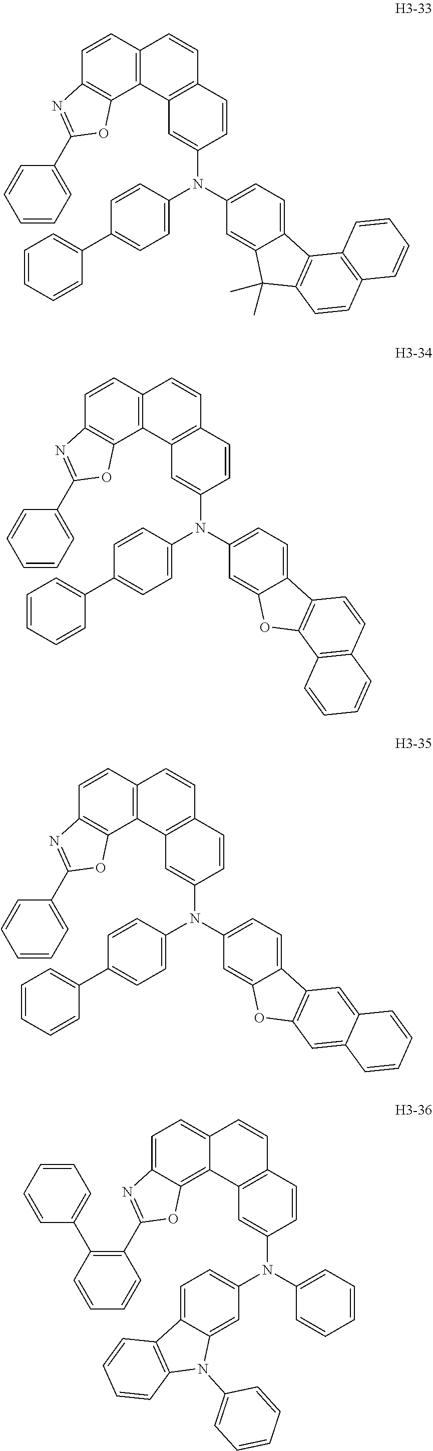 [SMILES: CC1(C)c2cc(N(c3ccc(-c4ccccc4)cc3)c3ccc4ccc5ccc6nc(-c7ccccc7)oc6c5c4c3)ccc2-c2c1ccc1ccccc21 ; c1ccc(-c2ccc(N(c3ccc4c(c3)oc3c5ccccc5ccc43)c3ccc4ccc5ccc6nc(-c7ccccc7)oc6c5c4c3)cc2)cc1 ; c1ccc(-c2ccc(N(c3ccc4c(c3)oc3cc5ccccc5cc34)c3ccc4ccc5ccc6nc(-c7ccccc7)oc6c5c4c3)cc2)cc1 ; c1ccc(-c2ccccc2-c2nc3ccc4ccc5ccc(N(c6ccccc6)c6ccc7c8ccccc8n(-c8ccccc8)c7c6)cc5c4c3o2)cc1]

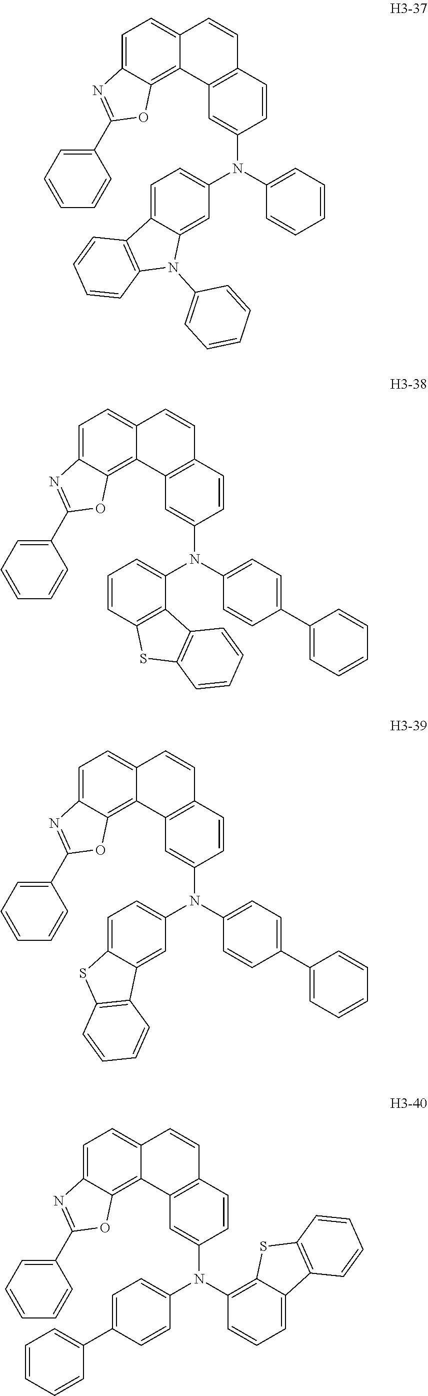 [SMILES: c1ccc(-c2nc3ccc4ccc5ccc(N(c6ccccc6)c6ccc7c8ccccc8n(-c8ccccc8)c7c6)cc5c4c3o2)cc1 ; c1ccc(-c2ccc(N(c3ccc4ccc5ccc6nc(-c7ccccc7)oc6c5c4c3)c3cccc4sc5ccccc5c34)cc2)cc1 ; c1ccc(-c2ccc(N(c3ccc4sc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4c3sc3ccccc34)cc2)cc1]

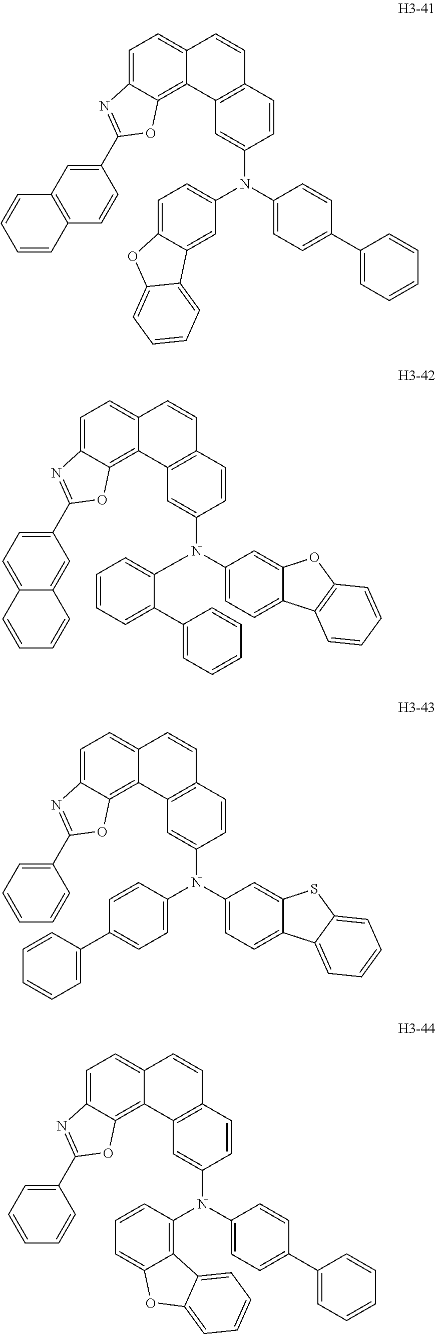 [SMILES: c1ccc(-c2ccc(N(c3ccc4oc5ccccc5c4c3)c3ccc4ccc5ccc6nc(-c7ccc8ccccc8c7)oc6c5c4c3)cc2)cc1 ; c1ccc(-c2ccccc2N(c2ccc3c(c2)oc2ccccc23)c2ccc3ccc4ccc5nc(-c6ccc7ccccc7c6)oc5c4c3c2)cc1 ; c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4oc5ccccc5c34)cc2)cc1]

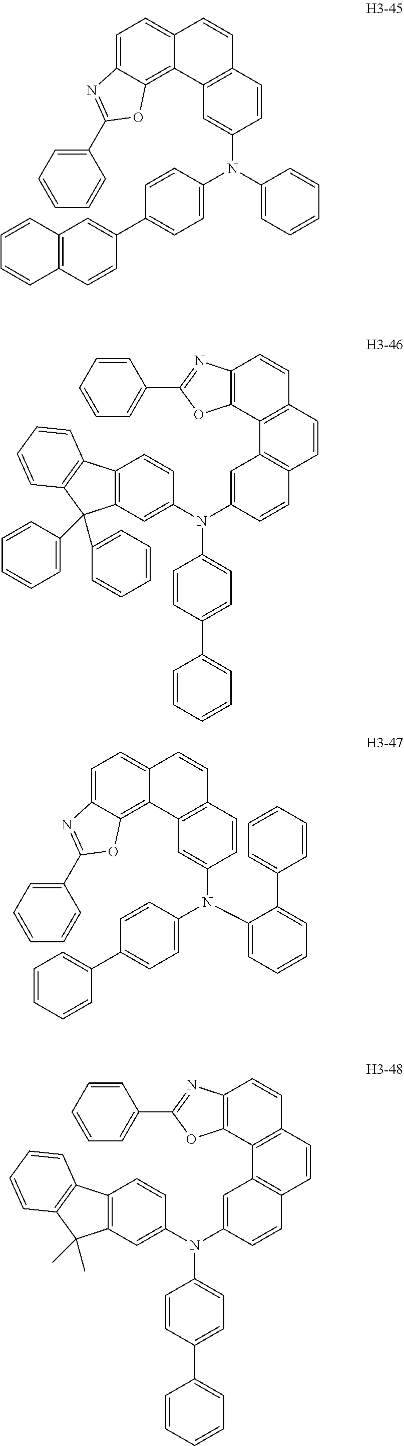

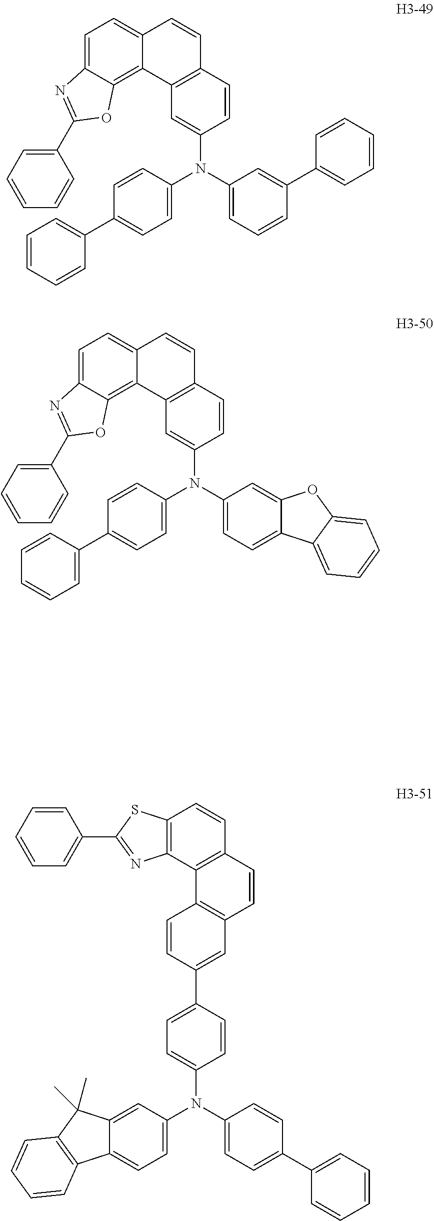 [SMILES: c1ccc(-c2ccc(N(c3cccc(-c4ccccc4)c3)c3ccc4ccc5ccc6nc(-c7ccccc7)oc6c5c4c3)cc2)cc1 ; c1ccc(-c2ccc(N(c3ccc4c(c3)oc3ccccc34)c3ccc4ccc5ccc6nc(-c7ccccc7)oc6c5c4c3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21]

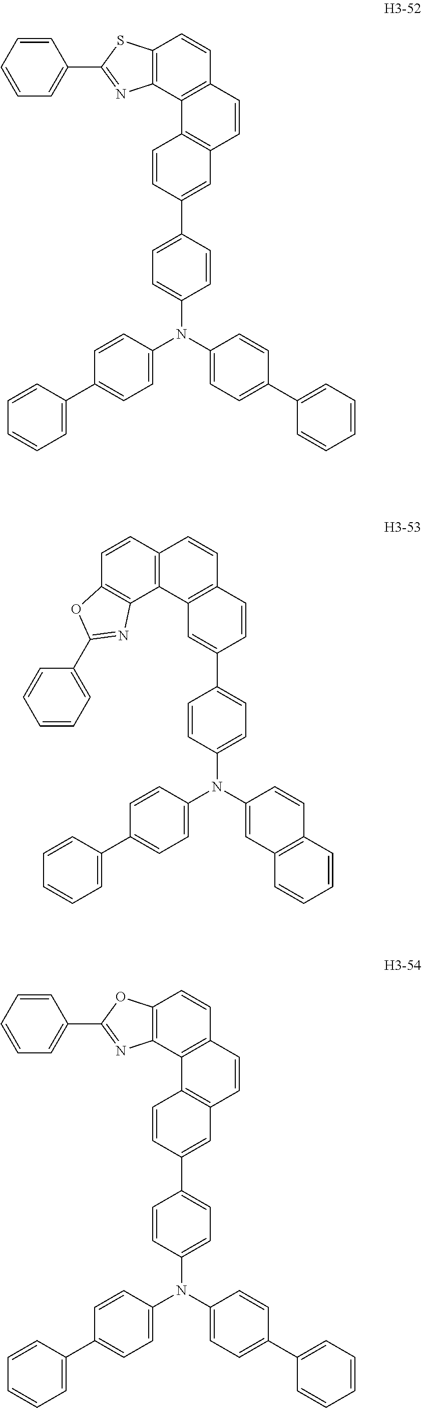 [SMILES: c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1]

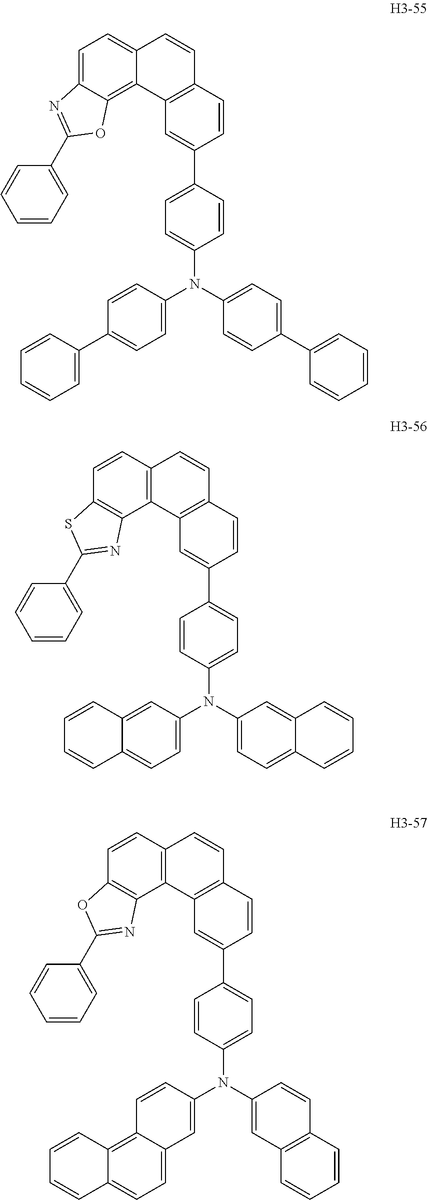 [SMILES: c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2nc3c(ccc4ccc5ccc(-c6ccc(N(c7ccc8ccccc8c7)c7ccc8ccccc8c7)cc6)cc5c43)s2)cc1 ; c1ccc(-c2nc3c(ccc4ccc5ccc(-c6ccc(N(c7ccc8ccccc8c7)c7ccc8c(ccc9ccccc98)c7)cc6)cc5c43)o2)cc1]

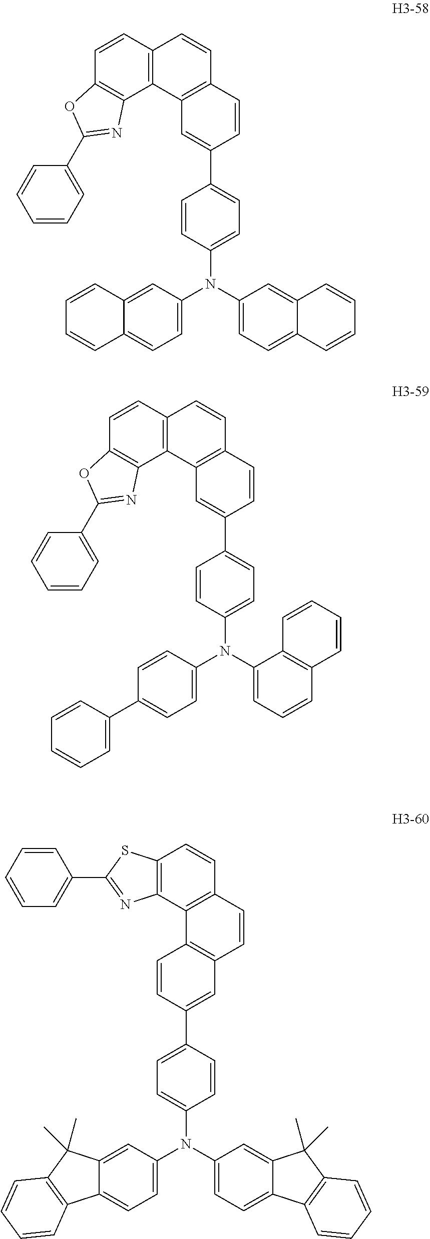 [SMILES: c1ccc(-c2nc3c(ccc4ccc5ccc(-c6ccc(N(c7ccc8ccccc8c7)c7ccc8ccccc8c7)cc6)cc5c43)o2)cc1 ; c1ccc(-c2ccc(N(c3ccc(-c4ccc5ccc6ccc7oc(-c8ccccc8)nc7c6c5c4)cc3)c3cccc4ccccc34)cc2)cc1 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21]

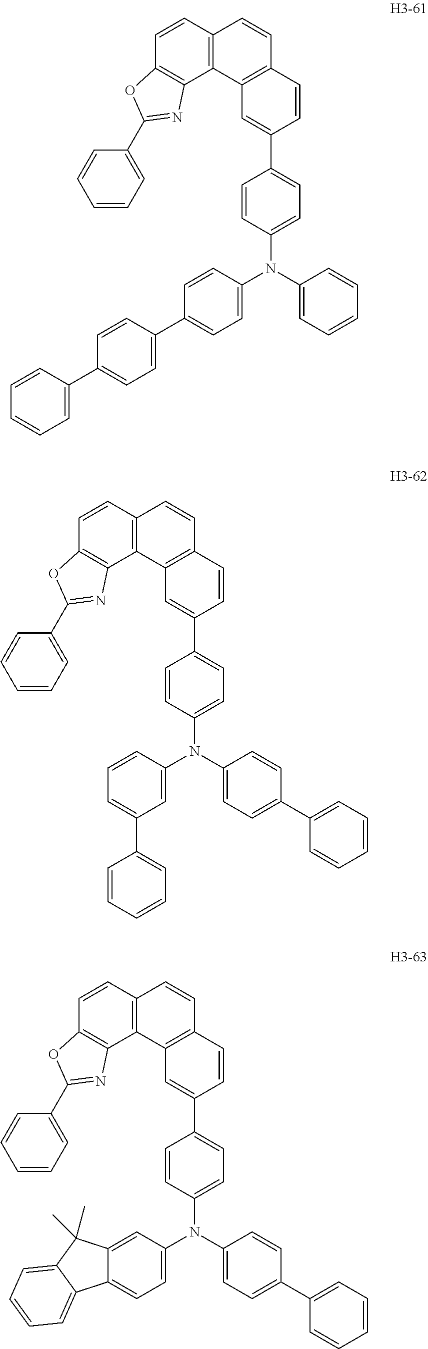 [SMILES: c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21]

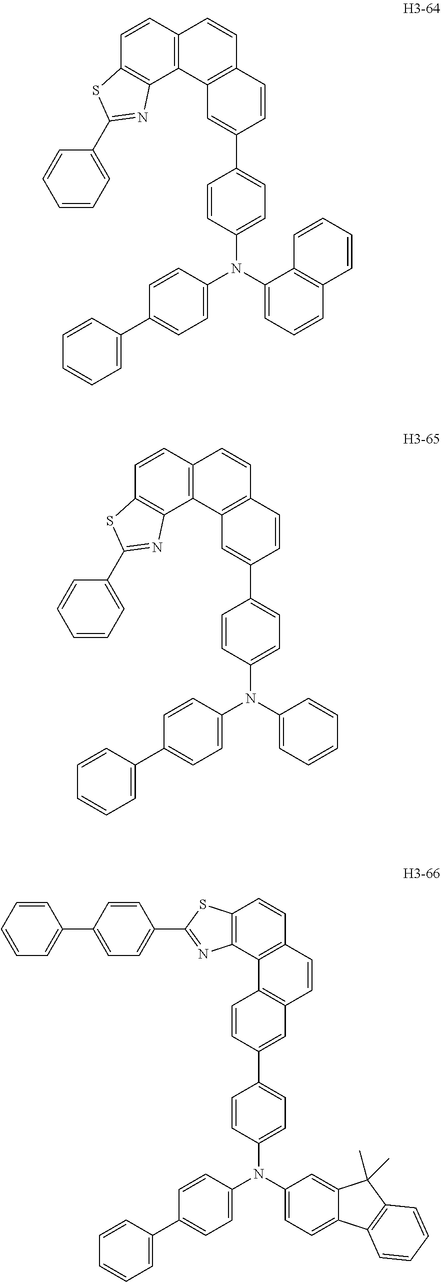 [SMILES: c1ccc(-c2ccc(N(c3ccc(-c4ccc5ccc6ccc7sc(-c8ccccc8)nc7c6c5c4)cc3)c3cccc4ccccc34)cc2)cc1 ; c1ccc(-c2ccc(N(c3ccccc3)c3ccc(-c4ccc5ccc6ccc7sc(-c8ccccc8)nc7c6c5c4)cc3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21]

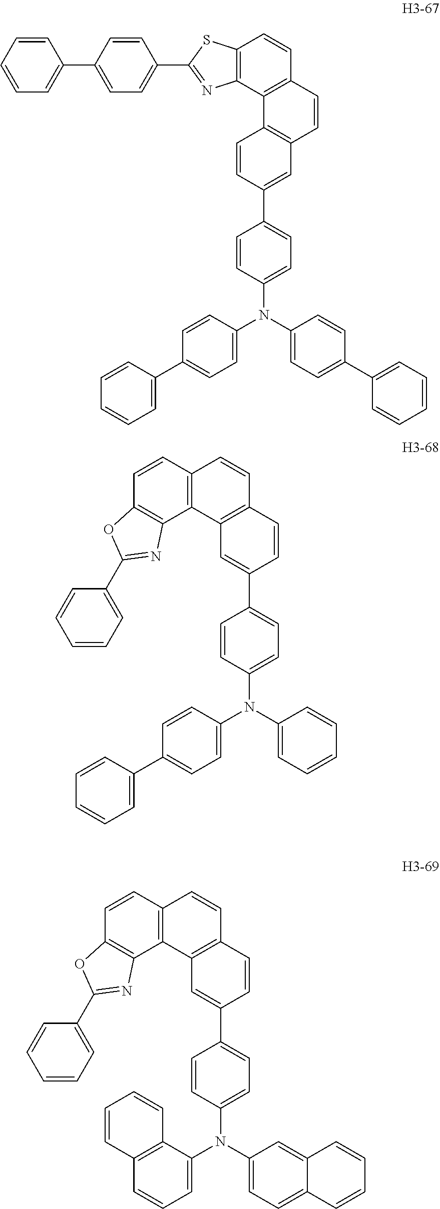 [SMILES: c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; c1ccc(-c2ccc(N(c3ccccc3)c3ccc(-c4ccc5ccc6ccc7oc(-c8ccccc8)nc7c6c5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1]

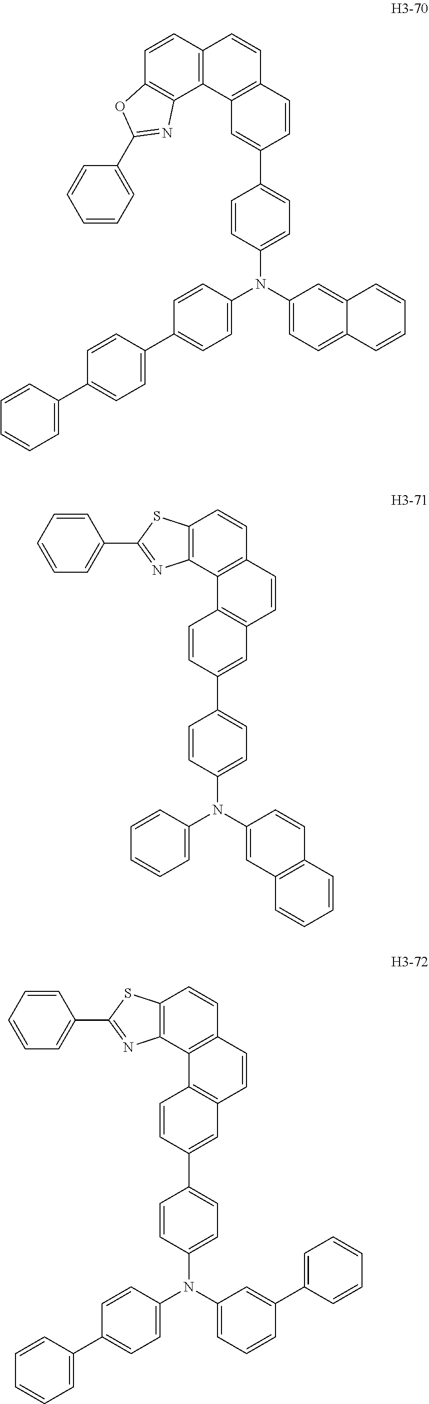 [SMILES: c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5cc(-c6ccc(N(c7ccccc7)c7ccc8ccccc8c7)cc6)ccc5c43)s2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1]

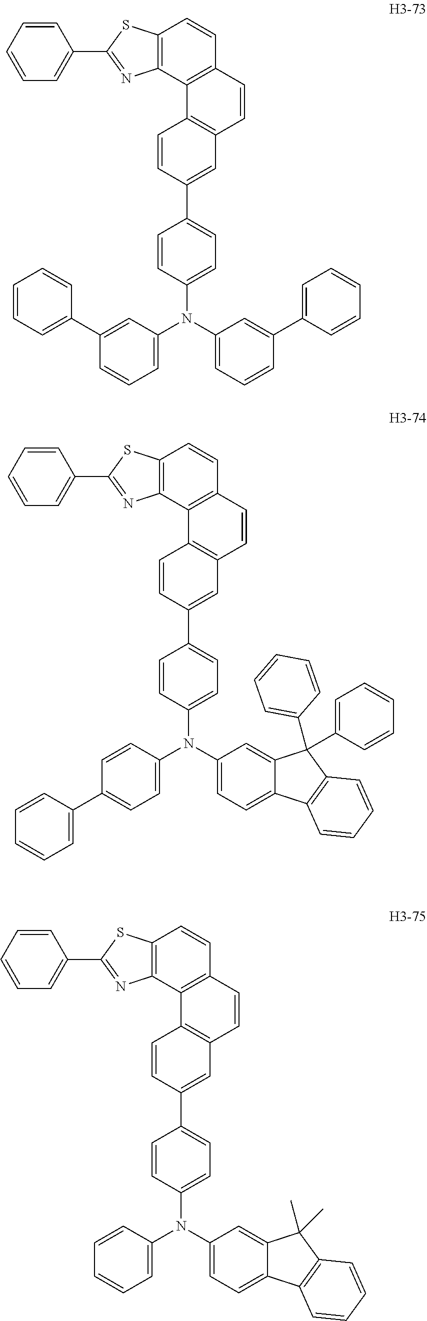 [SMILES: c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; CC1(C)c2ccccc2-c2ccc(N(c3ccccc3)c3ccc(-c4ccc5c(ccc6ccc7sc(-c8ccccc8)nc7c65)c4)cc3)cc21]

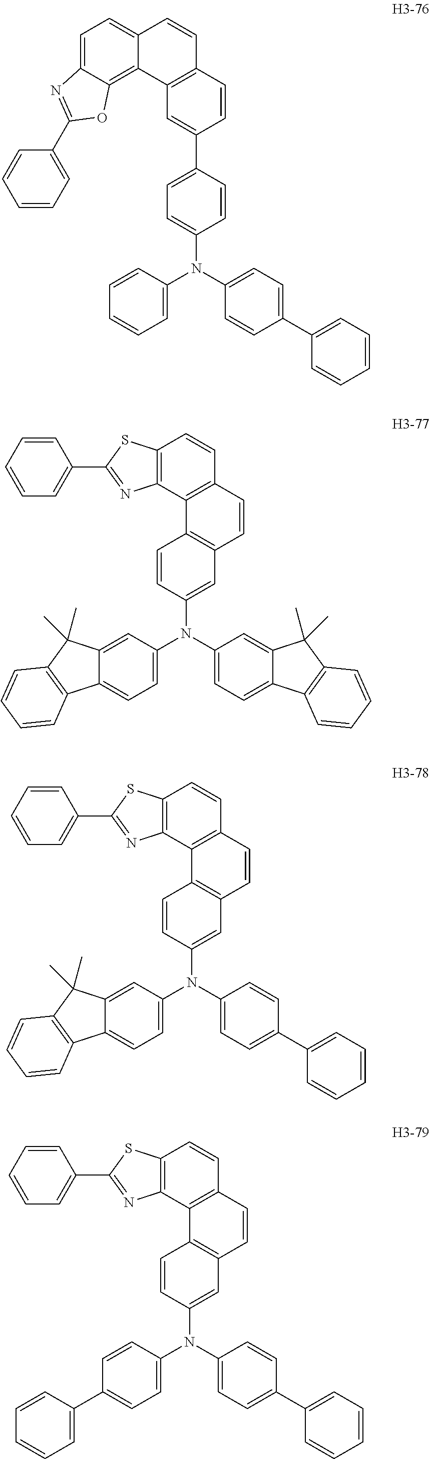 [SMILES: c1ccc(-c2ccc(N(c3ccccc3)c3ccc(-c4ccc5ccc6ccc7nc(-c8ccccc8)oc7c6c5c4)cc3)cc2)cc1 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; CC1(C)c2ccccc2-c2ccc(N(c3ccc(-c4ccccc4)cc3)c3ccc4c(ccc5ccc6sc(-c7ccccc7)nc6c54)c3)cc21 ; c1ccc(-c2ccc(N(c3ccc(-c4ccccc4)cc3)c3ccc4c(ccc5ccc6sc(-c7ccccc7)nc6c54)c3)cc2)cc1]

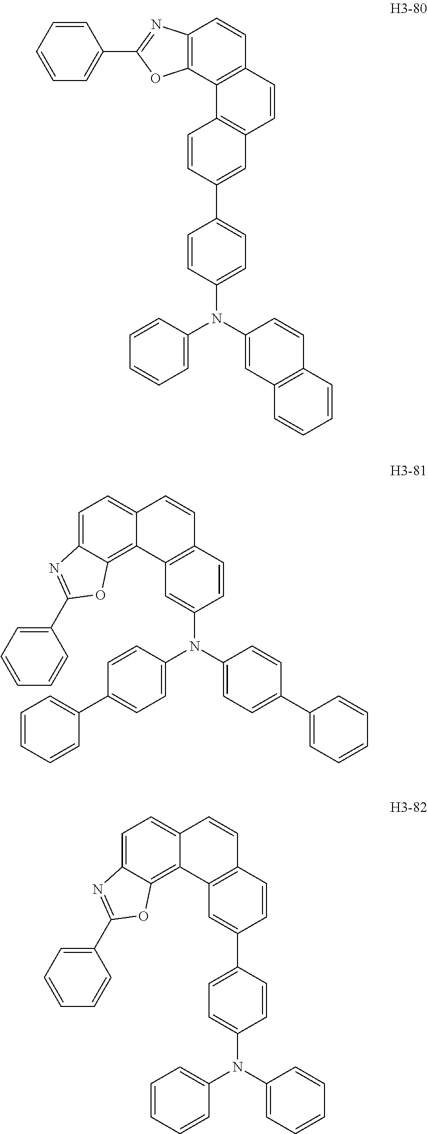 [SMILES: c1ccc(-c2nc3ccc4ccc5cc(-c6ccc(N(c7ccccc7)c7ccc8ccccc8c7)cc6)ccc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1 ; c1ccc(-c2nc3ccc4ccc5ccc(-c6ccc(N(c7ccccc7)c7ccccc7)cc6)cc5c4c3o2)cc1]

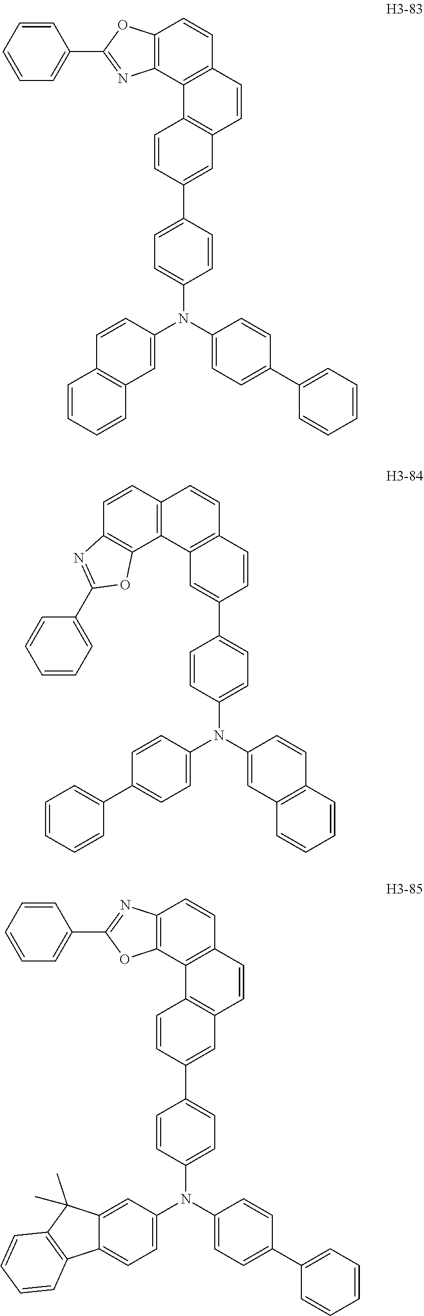 [SMILES: c1ccc(-c2ccc(N(c3ccc(-c4ccc5c(ccc6ccc7oc(-c8ccccc8)nc7c65)c4)cc3)c3ccc4ccccc4c3)cc2)cc1 ; c1ccc(-c2ccc(N(c3ccc(-c4ccc5ccc6ccc7nc(-c8ccccc8)oc7c6c5c4)cc3)c3ccc4ccccc4c3)cc2)cc1 ; CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21]

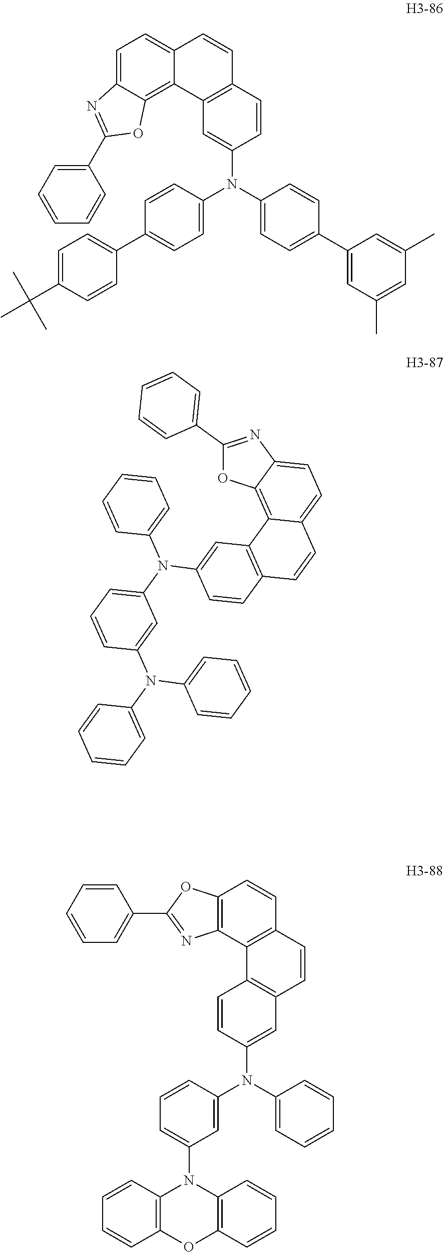 [SMILES: Cc1cc(C)cc(-c2ccc(N(c3ccc(-c4ccc(C(C)(C)C)cc4)cc3)c3ccc4ccc5ccc6nc(-c7ccccc7)oc6c5c4c3)cc2)c1 ; c1ccc(-c2nc3ccc4ccc5ccc(N(c6ccccc6)c6cccc(N(c7ccccc7)c7ccccc7)c6)cc5c4c3o2)cc1 ; c1ccc(-c2nc3c(ccc4ccc5cc(N(c6ccccc6)c6cccc(N7c8ccccc8Oc8ccccc87)c6)ccc5c43)o2)cc1]

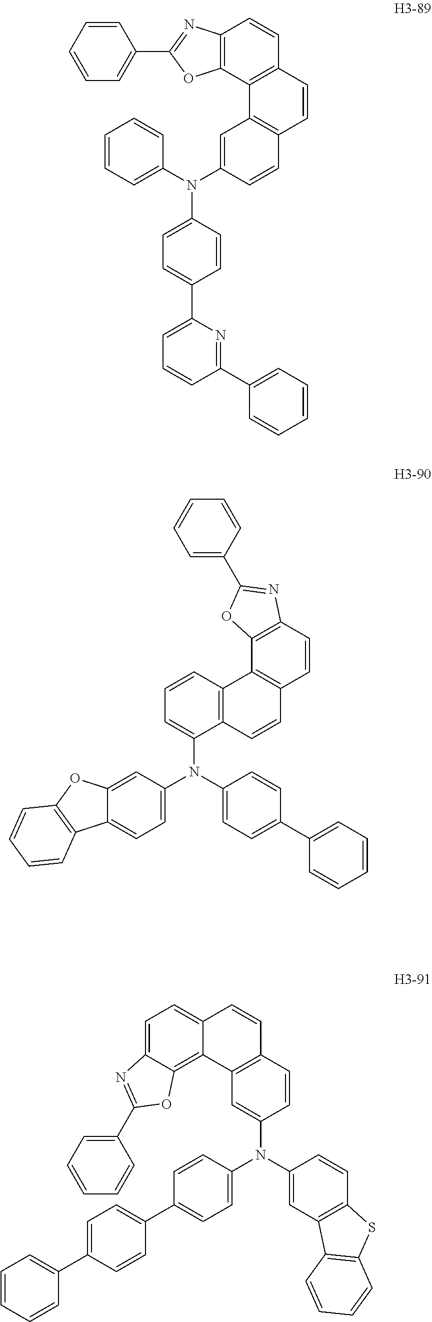 [SMILES: c1ccc(-c2cccc(-c3ccc(N(c4ccccc4)c4ccc5ccc6ccc7nc(-c8ccccc8)oc7c6c5c4)cc3)n2)cc1 ; c1ccc(-c2ccc(N(c3ccc4c(c3)oc3ccccc34)c3cccc4c3ccc3ccc5nc(-c6ccccc6)oc5c34)cc2)cc1 ; c1ccc(-c2ccc(-c3ccc(N(c4ccc5sc6ccccc6c5c4)c4ccc5ccc6ccc7nc(-c8ccccc8)oc7c6c5c4)cc3)cc2)cc1]

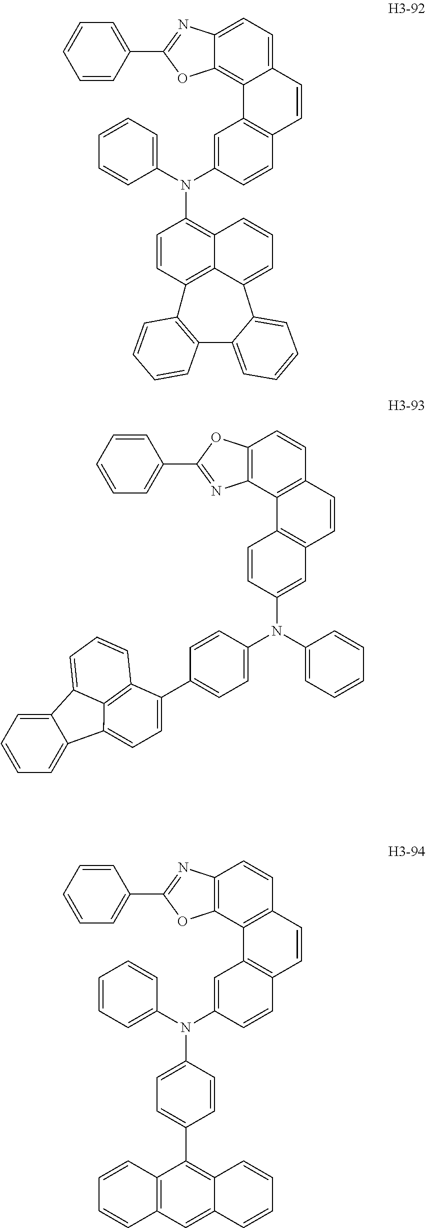 [SMILES: c1ccc(-c2nc3ccc4ccc5ccc(N(c6ccccc6)c6ccc7c8c(cccc68)-c6ccccc6-c6ccccc6-7)cc5c4c3o2)cc1 ; c1ccc(-c2nc3c(ccc4ccc5cc(N(c6ccccc6)c6ccc(-c7ccc8c9c(cccc79)-c7ccccc7-8)cc6)ccc5c43)o2)cc1 ; c1ccc(-c2nc3ccc4ccc5ccc(N(c6ccccc6)c6ccc(-c7c8ccccc8cc8ccccc78)cc6)cc5c4c3o2)cc1]

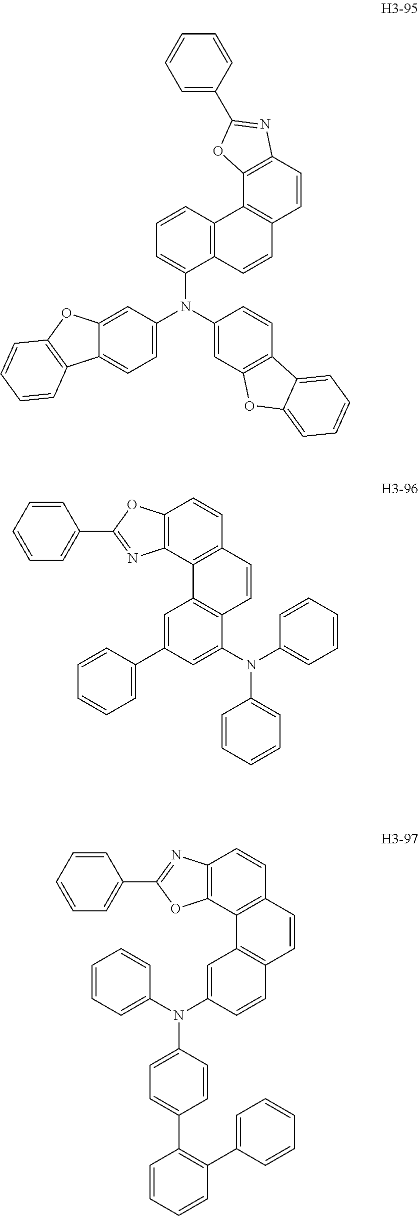 [SMILES: c1ccc(-c2nc3ccc4ccc5c(N(c6ccc7c(c6)oc6ccccc67)c6ccc7c(c6)oc6ccccc67)cccc5c4c3o2)cc1 ; c1ccc(-c2cc(N(c3ccccc3)c3ccccc3)c3ccc4ccc5oc(-c6ccccc6)nc5c4c3c2)cc1 ; c1ccc(-c2nc3ccc4ccc5ccc(N(c6ccccc6)c6ccc(-c7ccccc7-c7ccccc7)cc6)cc5c4c3o2)cc1]

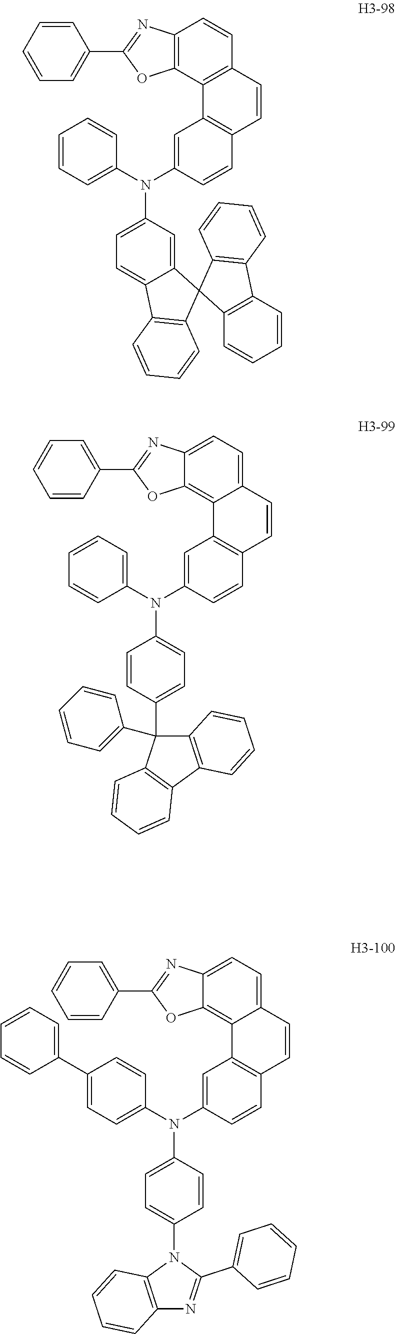 [SMILES: c1ccc(-c2nc3ccc4ccc5ccc(N(c6ccccc6)c6ccc7c(c6)C6(c8ccccc8-c8ccccc86)c6ccccc6-7)cc5c4c3o2)cc1 ; c1ccc(-c2nc3ccc4ccc5ccc(N(c6ccccc6)c6ccc(C7(c8ccccc8)c8ccccc8-c8ccccc87)cc6)cc5c4c3o2)cc1 ; c1ccc(-c2ccc(N(c3ccc(-n4c(-c5ccccc5)nc5ccccc54)cc3)c3ccc4ccc5ccc6nc(-c7ccccc7)oc6c5c4c3)cc2)cc1]

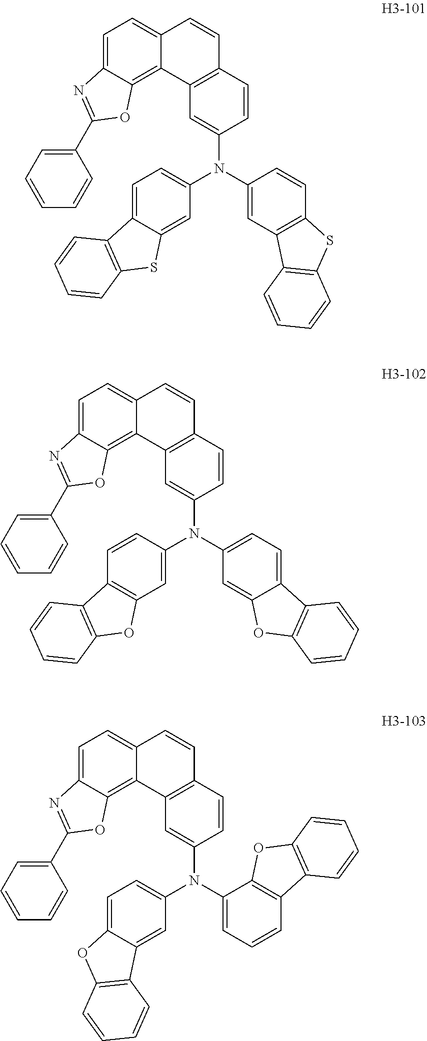 [SMILES: c1ccc(-c2nc3ccc4ccc5ccc(N(c6ccc7c(c6)sc6ccccc67)c6ccc7sc8ccccc8c7c6)cc5c4c3o2)cc1 ; c1ccc(-c2nc3ccc4ccc5ccc(N(c6ccc7c(c6)oc6ccccc67)c6ccc7c(c6)oc6ccccc67)cc5c4c3o2)cc1 ; c1ccc(-c2nc3ccc4ccc5ccc(N(c6ccc7oc8ccccc8c7c6)c6cccc7c6oc6ccccc67)cc5c4c3o2)cc1]

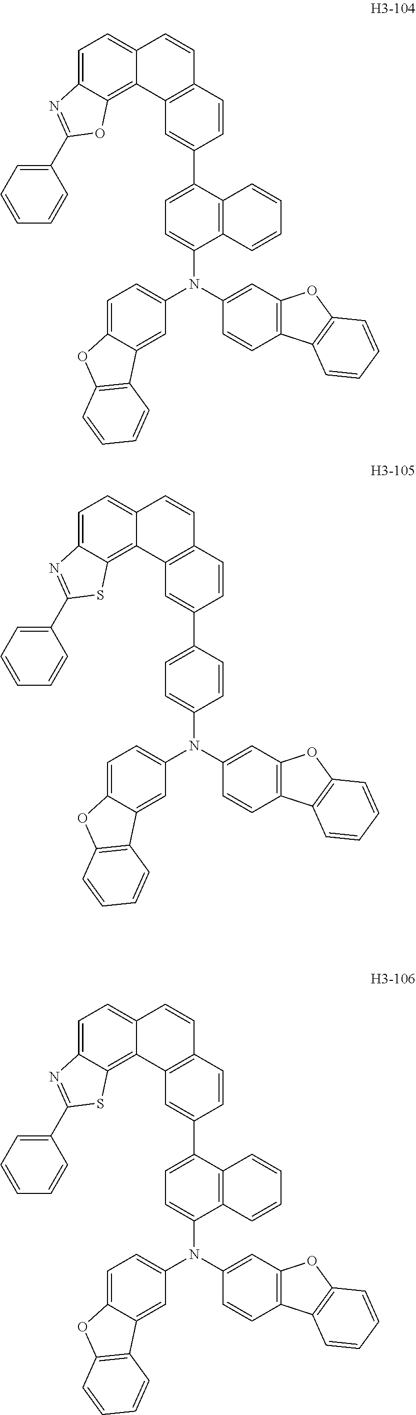 [SMILES: c1ccc(-c2nc3ccc4ccc5ccc(-c6ccc(N(c7ccc8c(c7)oc7ccccc78)c7ccc8oc9ccccc9c8c7)c7ccccc67)cc5c4c3o2)cc1 ; c1ccc(-c2nc3ccc4ccc5ccc(-c6ccc(N(c7ccc8c(c7)oc7ccccc78)c7ccc8oc9ccccc9c8c7)cc6)cc5c4c3s2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3s2)cc1]

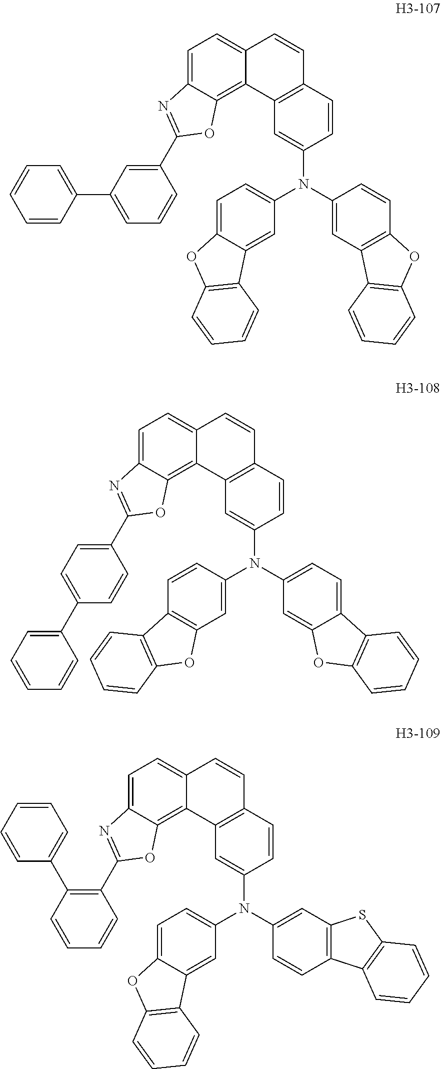 [SMILES: c1ccc(-c2cccc(-c3nc4ccc5ccc6ccc(N(c7ccc8oc9ccccc9c8c7)c7ccc8oc9ccccc9c8c7)cc6c5c4o3)c2)cc1 ; c1ccc(-c2ccc(-c3nc4ccc5ccc6ccc(N(c7ccc8c(c7)oc7ccccc78)c7ccc8c(c7)oc7ccccc78)cc6c5c4o3)cc2)cc1 ; c1ccc(-c2ccccc2-c2nc3ccc4ccc5ccc(N(c6ccc7c(c6)sc6ccccc67)c6ccc7oc8ccccc8c7c6)cc5c4c3o2)cc1]

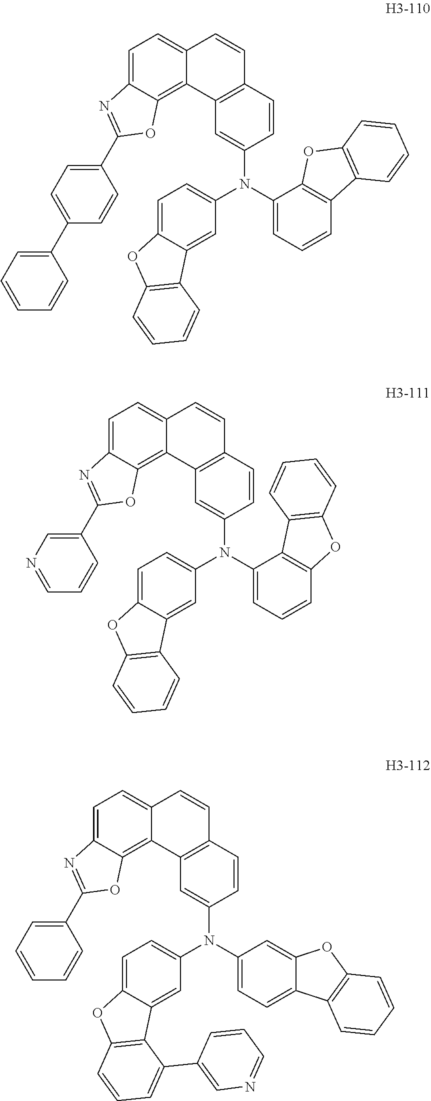 [SMILES: c1ccc(-c2ccc(-c3nc4ccc5ccc6ccc(N(c7ccc8oc9ccccc9c8c7)c7cccc8c7oc7ccccc78)cc6c5c4o3)cc2)cc1 ; c1cncc(-c2nc3ccc4ccc5ccc(N(c6ccc7oc8ccccc8c7c6)c6cccc7oc8ccccc8c67)cc5c4c3o2)c1 ; c1ccc(-c2nc3ccc4ccc5ccc(N(c6ccc7c(c6)oc6ccccc67)c6ccc7oc8cccc(-c9cccnc9)c8c7c6)cc5c4c3o2)cc1]

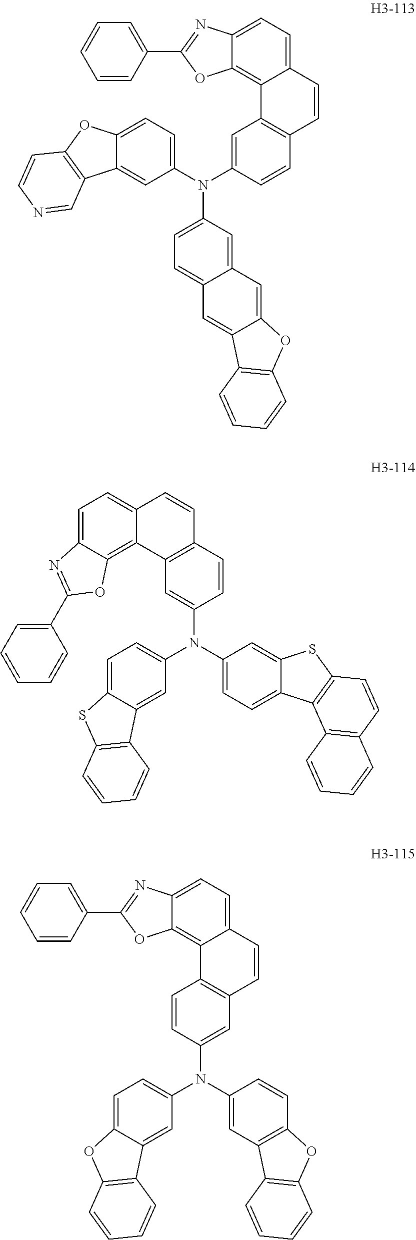 [SMILES: c1ccc(-c2nc3ccc4ccc5ccc(N(c6ccc7cc8c(cc7c6)oc6ccccc68)c6ccc7oc8ccncc8c7c6)cc5c4c3o2)cc1 ; c1ccc(-c2nc3ccc4ccc5ccc(N(c6ccc7c(c6)sc6ccc8ccccc8c67)c6ccc7sc8ccccc8c7c6)cc5c4c3o2)cc1 ; c1ccc(-c2nc3ccc4ccc5cc(N(c6ccc7oc8ccccc8c7c6)c6ccc7oc8ccccc8c7c6)ccc5c4c3o2)cc1]

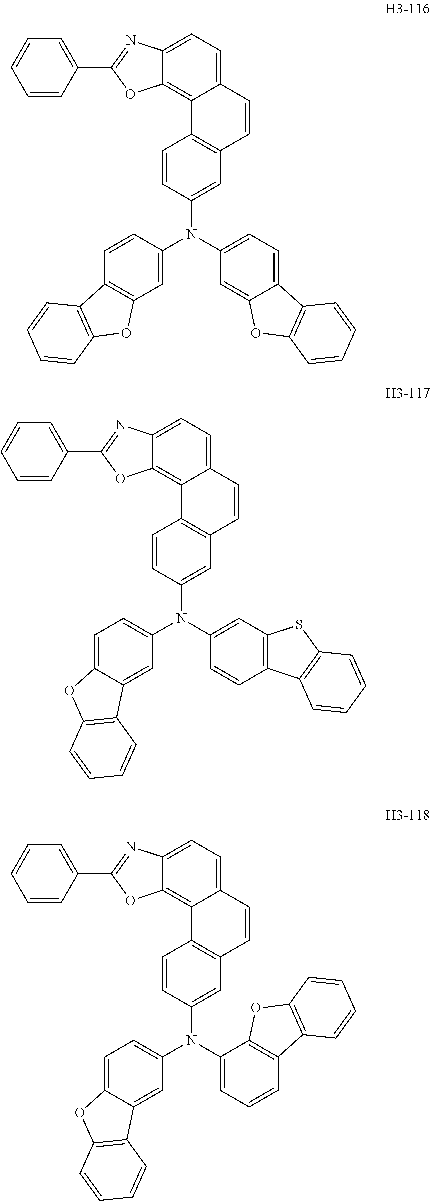 [SMILES: c1ccc(-c2nc3ccc4ccc5cc(N(c6ccc7c(c6)oc6ccccc67)c6ccc7c(c6)oc6ccccc67)ccc5c4c3o2)cc1 ; c1ccc(-c2nc3ccc4ccc5cc(N(c6ccc7c(c6)sc6ccccc67)c6ccc7oc8ccccc8c7c6)ccc5c4c3o2)cc1 ; c1ccc(-c2nc3ccc4ccc5cc(N(c6ccc7oc8ccccc8c7c6)c6cccc7c6oc6ccccc67)ccc5c4c3o2)cc1]

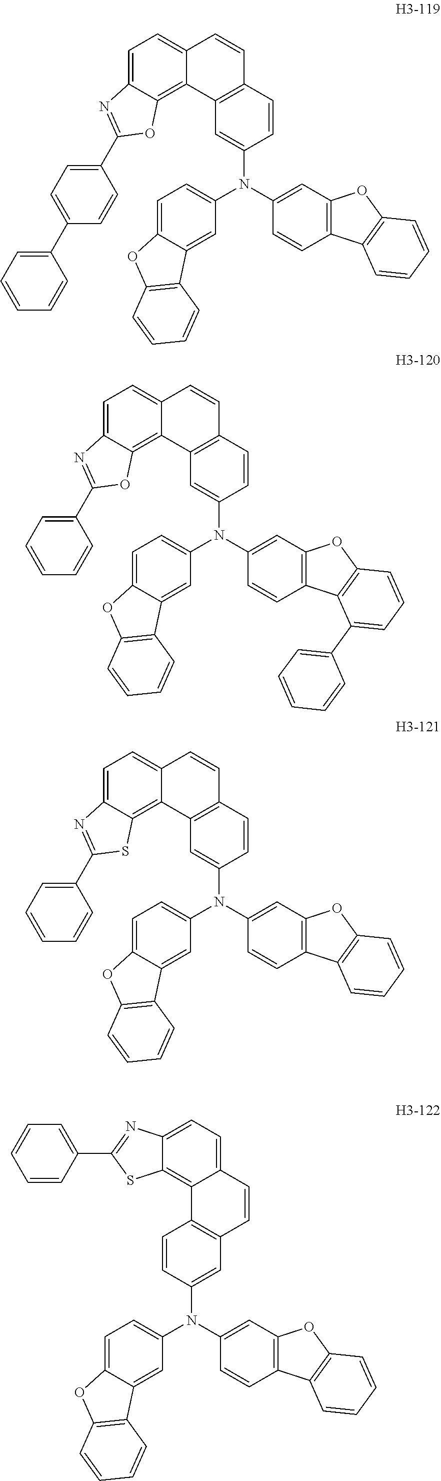

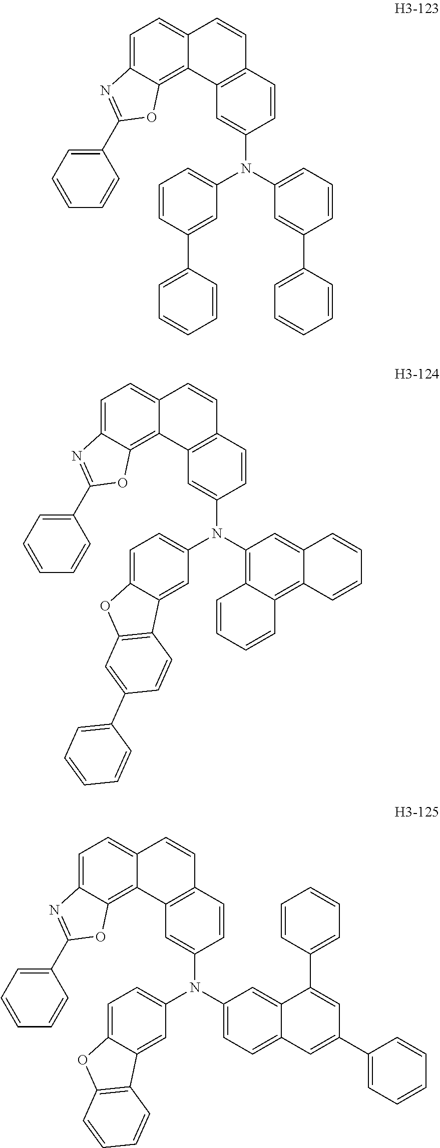 [SMILES: c1ccc(-c2cccc(N(c3cccc(-c4ccccc4)c3)c3ccc4ccc5ccc6nc(-c7ccccc7)oc6c5c4c3)c2)cc1 ; c1ccc(-c2ccc3c(c2)oc2ccc(N(c4ccc5ccc6ccc7nc(-c8ccccc8)oc7c6c5c4)c4cc5ccccc5c5ccccc45)cc23)cc1 ; c1ccc(-c2cc(-c3ccccc3)c3cc(N(c4ccc5oc6ccccc6c5c4)c4ccc5ccc6ccc7nc(-c8ccccc8)oc7c6c5c4)ccc3c2)cc1]

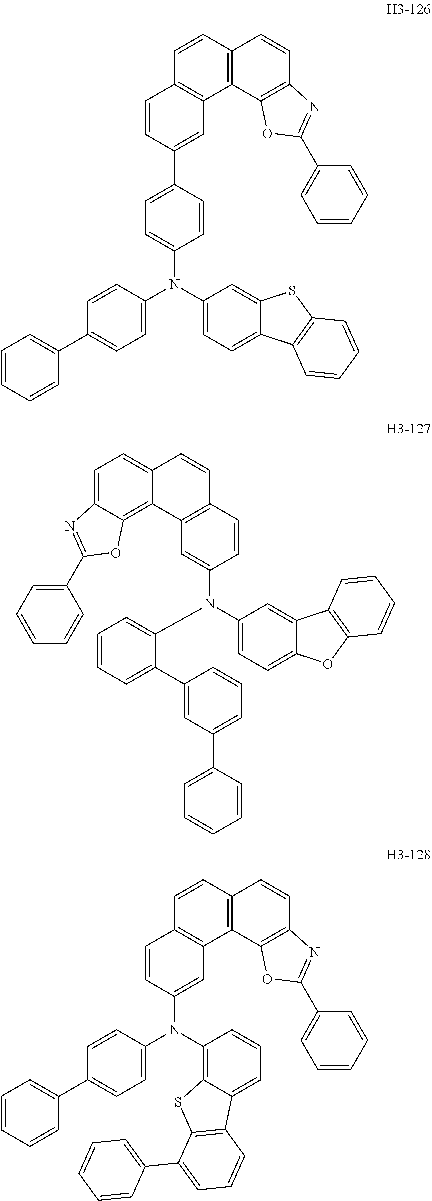 [SMILES: c1ccc(-c2ccc(N(c3ccc(-c4ccc5ccc6ccc7nc(-c8ccccc8)oc7c6c5c4)cc3)c3ccc4c(c3)sc3ccccc34)cc2)cc1 ; c1ccc(-c2cccc(-c3ccccc3N(c3ccc4oc5ccccc5c4c3)c3ccc4ccc5ccc6nc(-c7ccccc7)oc6c5c4c3)c2)cc1 ; c1ccc(-c2ccc(N(c3ccc4ccc5ccc6nc(-c7ccccc7)oc6c5c4c3)c3cccc4c3sc3c(-c5ccccc5)cccc34)cc2)cc1]

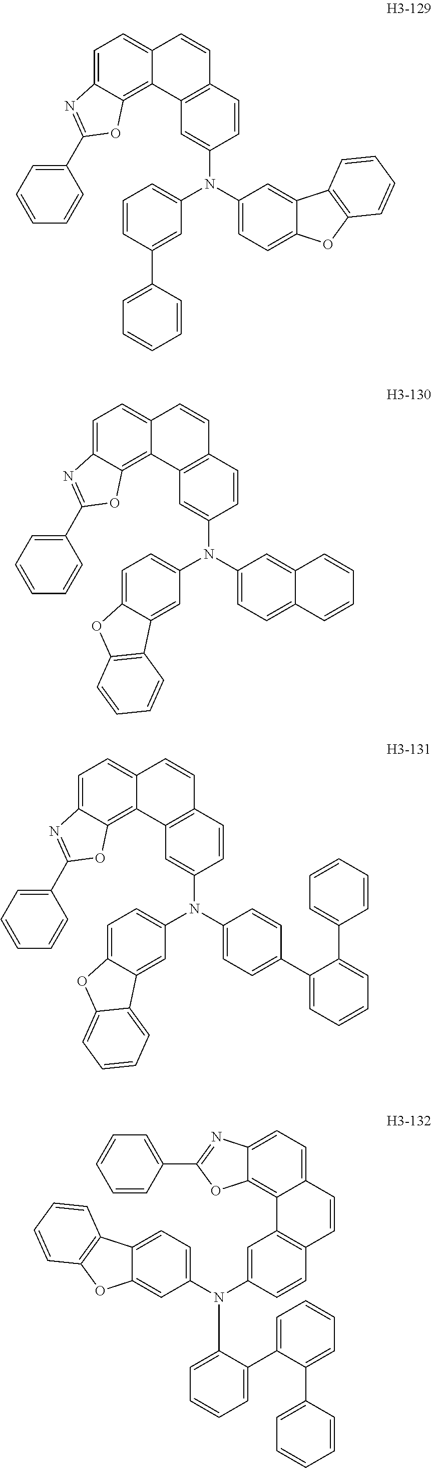

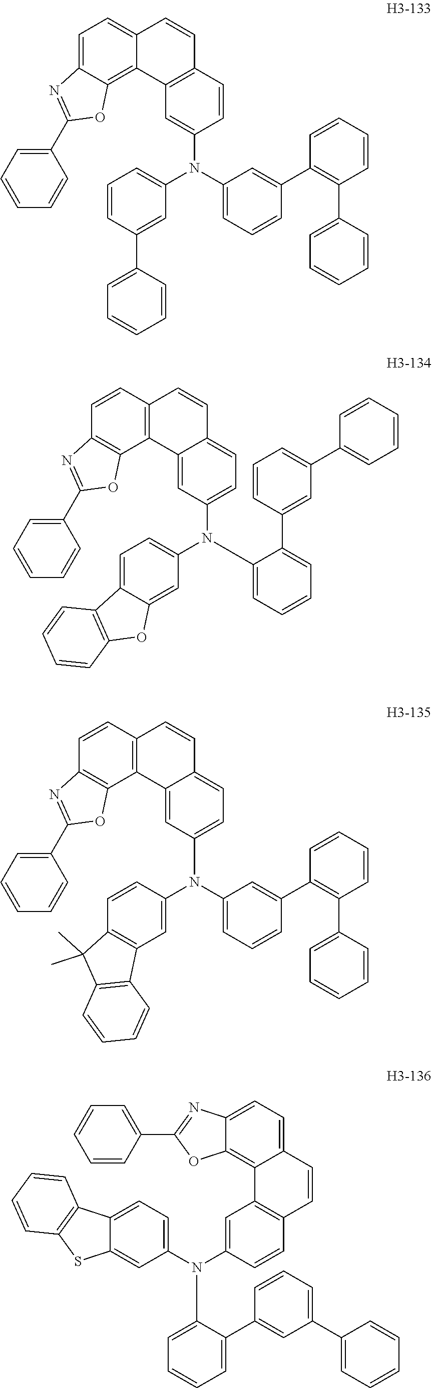 [SMILES: c1ccc(-c2cccc(N(c3cccc(-c4ccccc4-c4ccccc4)c3)c3ccc4ccc5ccc6nc(-c7ccccc7)oc6c5c4c3)c2)cc1 ; c1ccc(-c2cccc(-c3ccccc3N(c3ccc4c(c3)oc3ccccc34)c3ccc4ccc5ccc6nc(-c7ccccc7)oc6c5c4c3)c2)cc1 ; CC1(C)c2ccccc2-c2cc(N(c3cccc(-c4ccccc4-c4ccccc4)c3)c3ccc4ccc5ccc6nc(-c7ccccc7)oc6c5c4c3)ccc21 ; c1ccc(-c2cccc(-c3ccccc3N(c3ccc4c(c3)sc3ccccc34)c3ccc4ccc5ccc6nc(-c7ccccc7)oc6c5c4c3)c2)cc1]

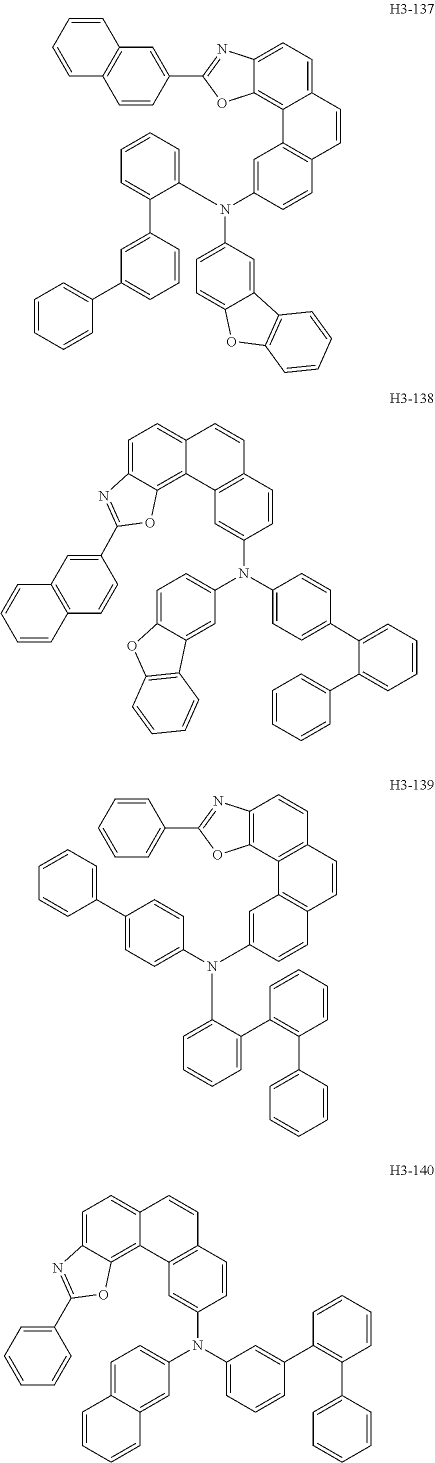 [SMILES: c1ccc(-c2cccc(-c3ccccc3N(c3ccc4oc5ccccc5c4c3)c3ccc4ccc5ccc6nc(-c7ccc8ccccc8c7)oc6c5c4c3)c2)cc1 ; c1ccc(-c2ccccc2-c2ccc(N(c3ccc4oc5ccccc5c4c3)c3ccc4ccc5ccc6nc(-c7ccc8ccccc8c7)oc6c5c4c3)cc2)cc1 ; c1ccc(-c2ccc(N(c3ccc4ccc5ccc6nc(-c7ccccc7)oc6c5c4c3)c3ccccc3-c3ccccc3-c3ccccc3)cc2)cc1 ; c1ccc(-c2nc3ccc4ccc5ccc(N(c6cccc(-c7ccccc7-c7ccccc7)c6)c6ccc7ccccc7c6)cc5c4c3o2)cc1]

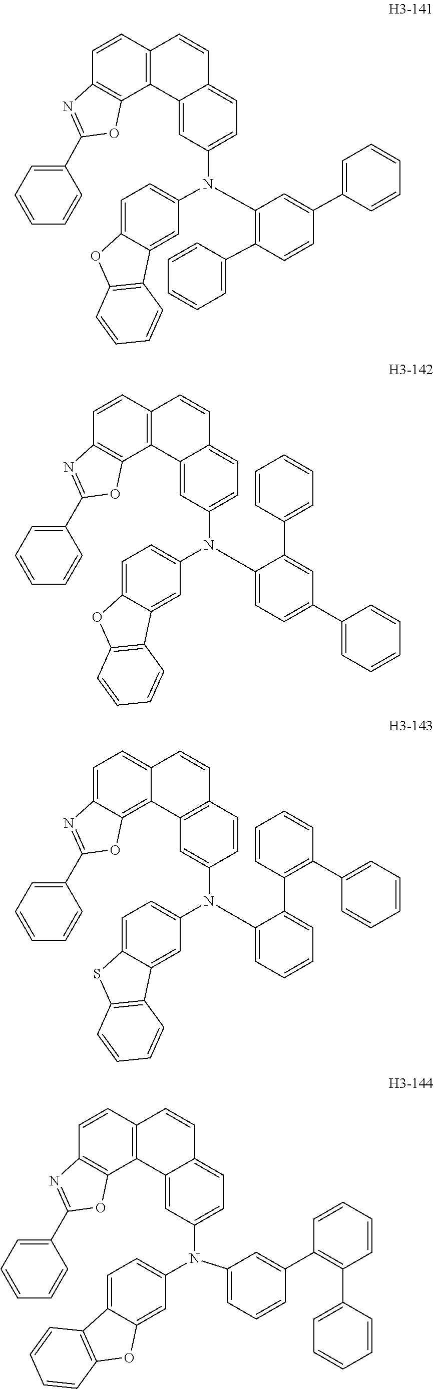

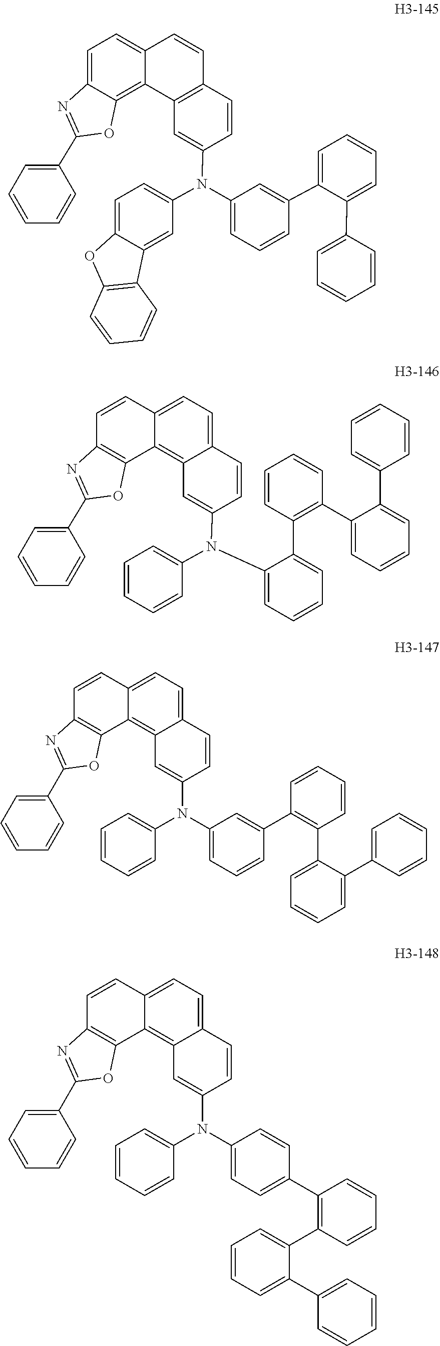 [SMILES: c1ccc(-c2nc3ccc4ccc5ccc(N(c6cccc(-c7ccccc7-c7ccccc7)c6)c6ccc7oc8ccccc8c7c6)cc5c4c3o2)cc1 ; c1ccc(-c2nc3ccc4ccc5ccc(N(c6ccccc6)c6ccccc6-c6ccccc6-c6ccccc6-c6ccccc6)cc5c4c3o2)cc1 ; c1ccc(-c2nc3ccc4ccc5ccc(N(c6ccccc6)c6cccc(-c7ccccc7-c7ccccc7-c7ccccc7)c6)cc5c4c3o2)cc1 ; c1ccc(-c2nc3ccc4ccc5ccc(N(c6ccccc6)c6ccc(-c7ccccc7-c7ccccc7-c7ccccc7)cc6)cc5c4c3o2)cc1]

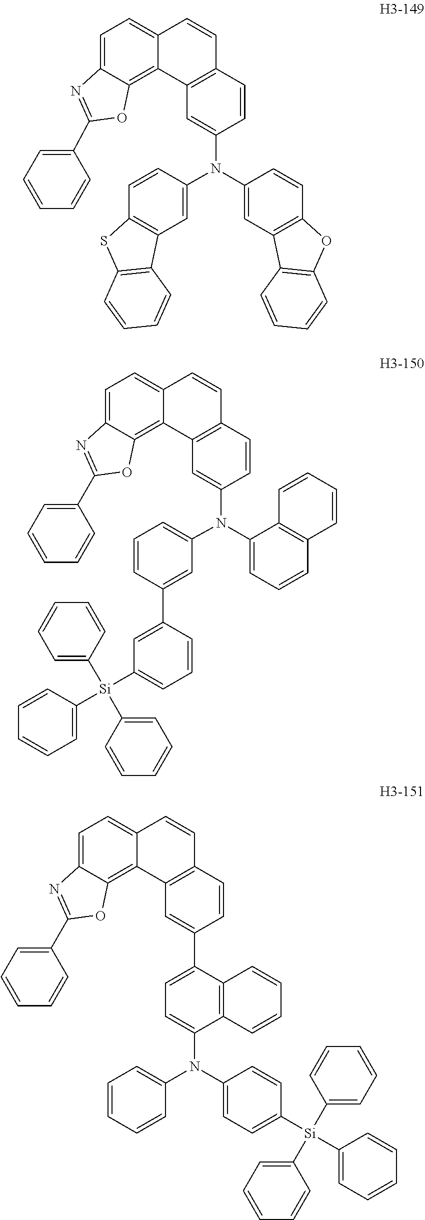

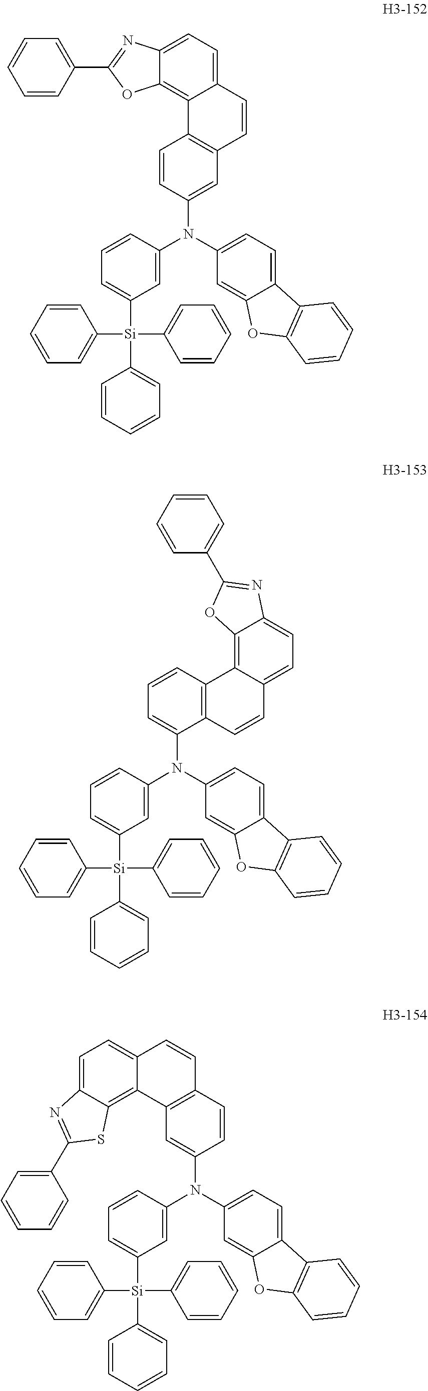 [SMILES: c1ccc(-c2nc3ccc4ccc5cc(N(c6cccc([Si](c7ccccc7)(c7ccccc7)c7ccccc7)c6)c6ccc7c(c6)oc6ccccc67)ccc5c4c3o2)cc1 ; c1ccc(-c2nc3ccc4ccc5c(N(c6cccc([Si](c7ccccc7)(c7ccccc7)c7ccccc7)c6)c6ccc7c(c6)oc6ccccc67)cccc5c4c3o2)cc1 ; c1ccc(-c2nc3ccc4ccc5ccc(N(c6cccc([Si](c7ccccc7)(c7ccccc7)c7ccccc7)c6)c6ccc7c(c6)oc6ccccc67)cc5c4c3s2)cc1]

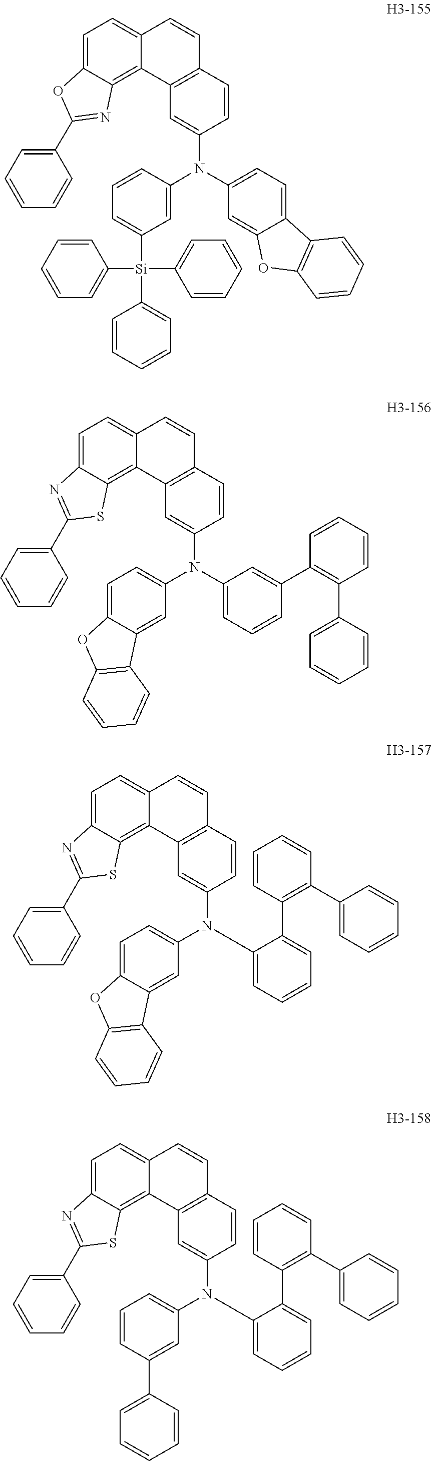 [SMILES: c1ccc(-c2nc3c(ccc4ccc5ccc(N(c6cccc([Si](c7ccccc7)(c7ccccc7)c7ccccc7)c6)c6ccc7c(c6)oc6ccccc67)cc5c43)o2)cc1 ; c1ccc(-c2nc3ccc4ccc5ccc(N(c6cccc(-c7ccccc7-c7ccccc7)c6)c6ccc7oc8ccccc8c7c6)cc5c4c3s2)cc1 ; c1ccc(-c2nc3ccc4ccc5ccc(N(c6ccc7oc8ccccc8c7c6)c6ccccc6-c6ccccc6-c6ccccc6)cc5c4c3s2)cc1 ; c1ccc(-c2cccc(N(c3ccc4ccc5ccc6nc(-c7ccccc7)sc6c5c4c3)c3ccccc3-c3ccccc3-c3ccccc3)c2)cc1]

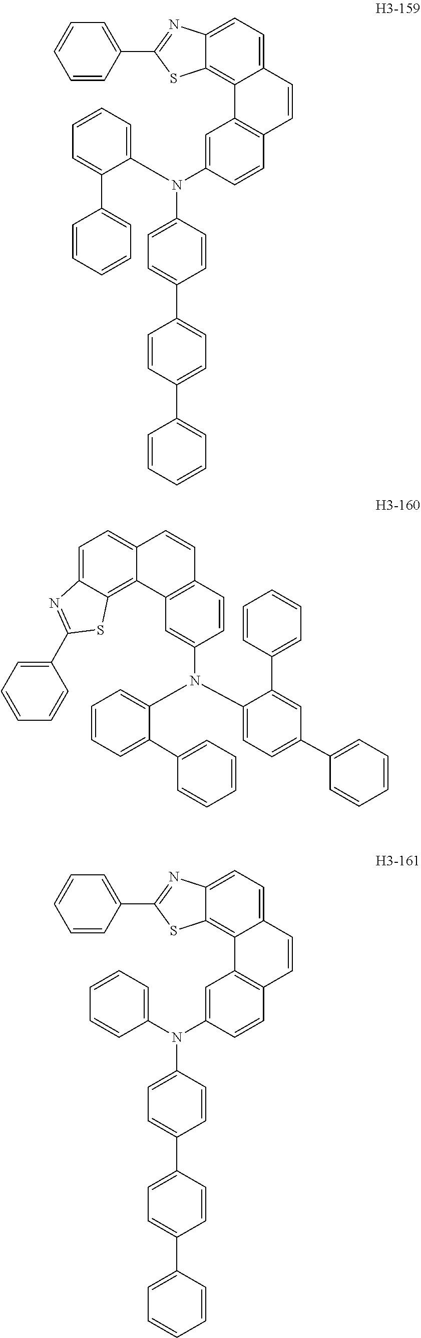 [SMILES: c1ccc(-c2ccc(-c3ccc(N(c4ccc5ccc6ccc7nc(-c8ccccc8)sc7c6c5c4)c4ccccc4-c4ccccc4)cc3)cc2)cc1 ; c1ccc(-c2ccc(N(c3ccc4ccc5ccc6nc(-c7ccccc7)sc6c5c4c3)c3ccccc3-c3ccccc3)c(-c3ccccc3)c2)cc1 ; c1ccc(-c2ccc(-c3ccc(N(c4ccccc4)c4ccc5ccc6ccc7nc(-c8ccccc8)sc7c6c5c4)cc3)cc2)cc1]

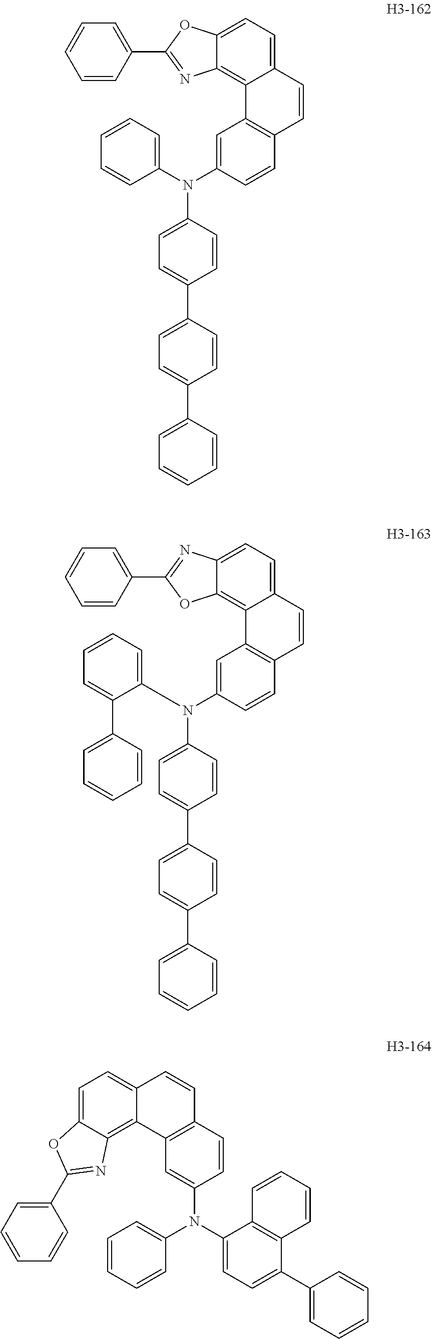 [SMILES: c1ccc(-c2ccc(-c3ccc(N(c4ccccc4)c4ccc5ccc6ccc7oc(-c8ccccc8)nc7c6c5c4)cc3)cc2)cc1 ; c1ccc(-c2ccc(-c3ccc(N(c4ccc5ccc6ccc7nc(-c8ccccc8)oc7c6c5c4)c4ccccc4-c4ccccc4)cc3)cc2)cc1 ; c1ccc(-c2nc3c(ccc4ccc5ccc(N(c6ccccc6)c6ccc(-c7ccccc7)c7ccccc67)cc5c43)o2)cc1]

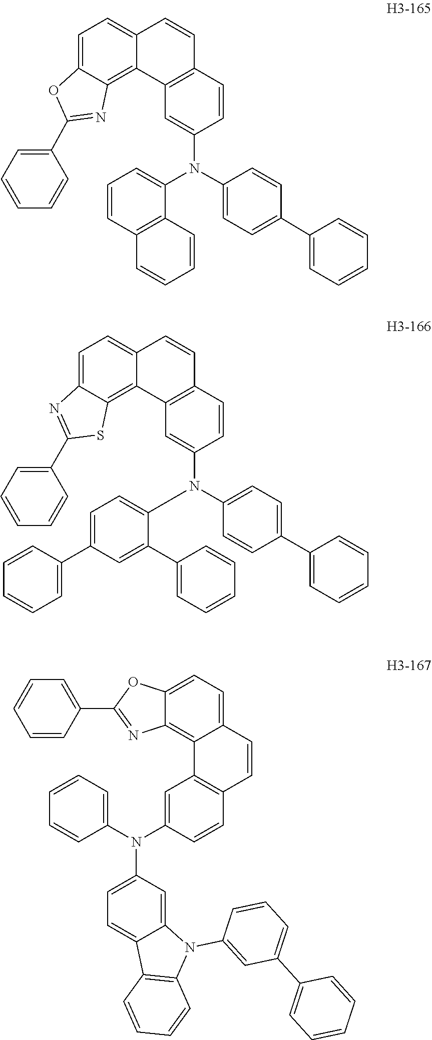 [SMILES: c1ccc(-c2ccc(N(c3ccc4ccc5ccc6oc(-c7ccccc7)nc6c5c4c3)c3cccc4ccccc34)cc2)cc1 ; c1ccc(-c2ccc(N(c3ccc4ccc5ccc6nc(-c7ccccc7)sc6c5c4c3)c3ccc(-c4ccccc4)cc3-c3ccccc3)cc2)cc1 ; c1ccc(-c2cccc(-n3c4ccccc4c4ccc(N(c5ccccc5)c5ccc6ccc7ccc8oc(-c9ccccc9)nc8c7c6c5)cc43)c2)cc1]

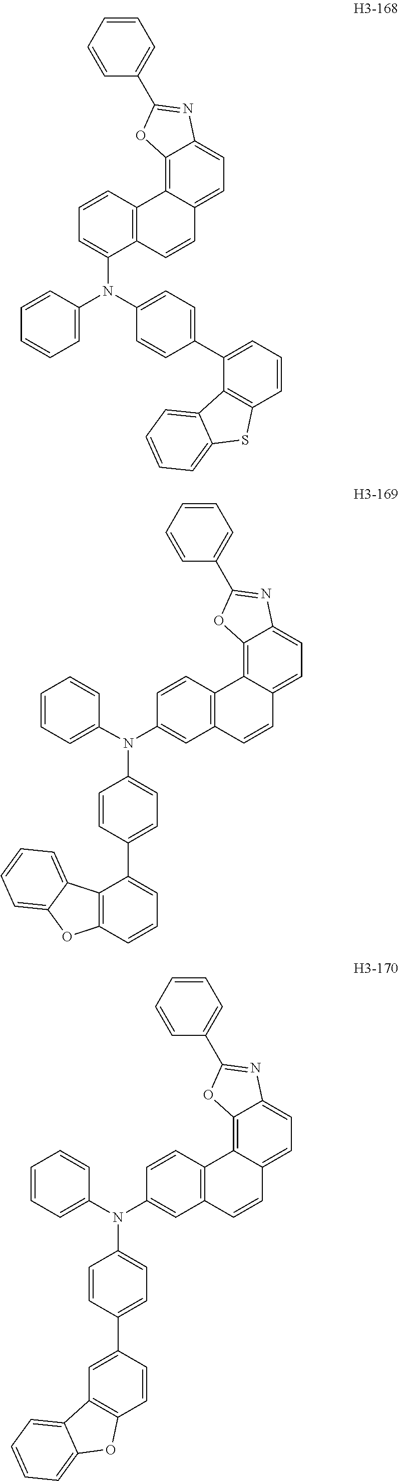

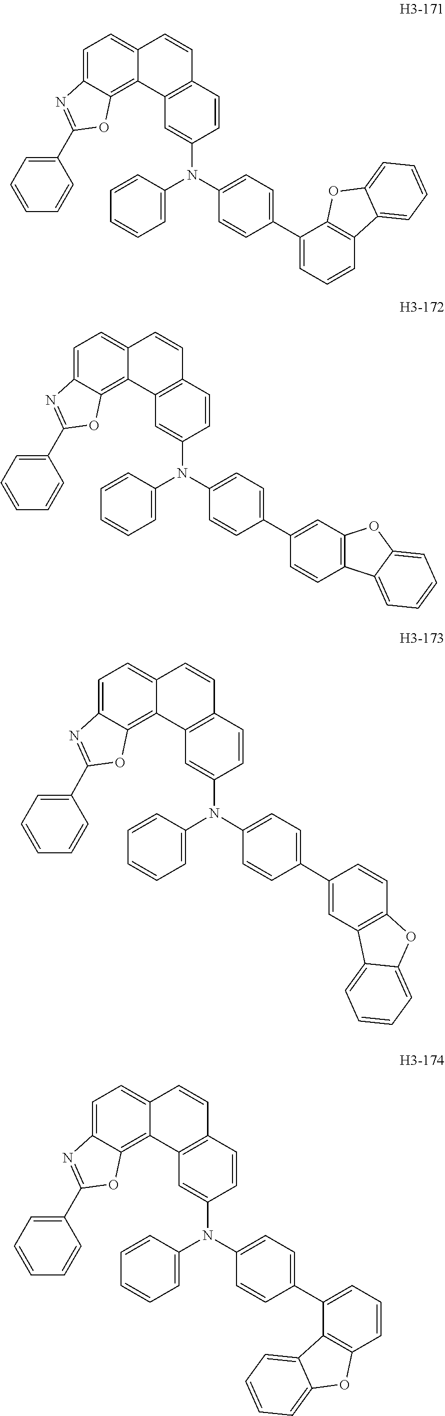 [SMILES: c1ccc(-c2nc3ccc4ccc5ccc(N(c6ccccc6)c6ccc(-c7cccc8c7oc7ccccc78)cc6)cc5c4c3o2)cc1 ; c1ccc(-c2nc3ccc4ccc5ccc(N(c6ccccc6)c6ccc(-c7ccc8c(c7)oc7ccccc78)cc6)cc5c4c3o2)cc1 ; c1ccc(-c2nc3ccc4ccc5ccc(N(c6ccccc6)c6ccc(-c7ccc8oc9ccccc9c8c7)cc6)cc5c4c3o2)cc1 ; c1ccc(-c2nc3ccc4ccc5ccc(N(c6ccccc6)c6ccc(-c7cccc8oc9ccccc9c78)cc6)cc5c4c3o2)cc1]

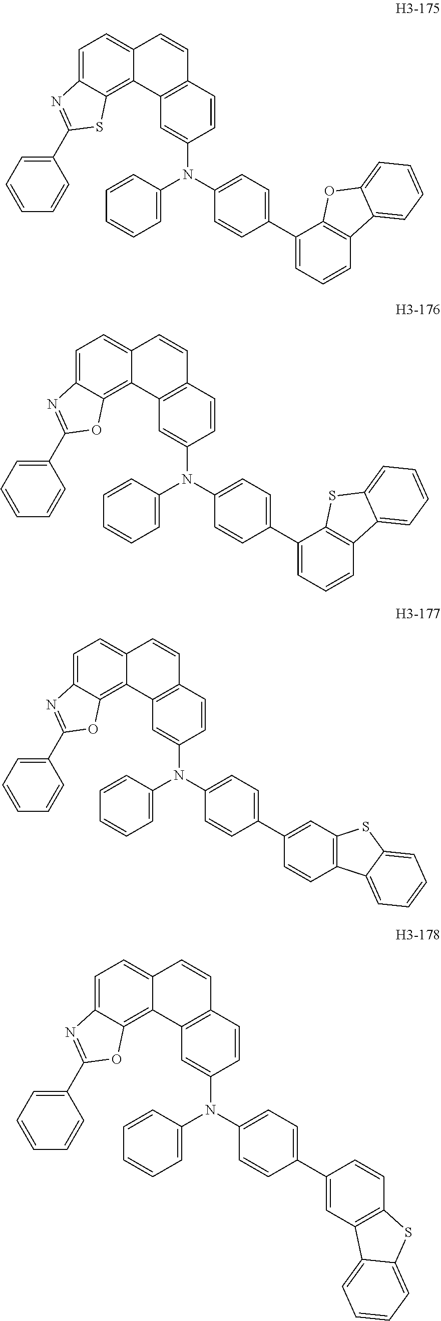 [SMILES: c1ccc(-c2nc3ccc4ccc5ccc(N(c6ccccc6)c6ccc(-c7cccc8c7oc7ccccc78)cc6)cc5c4c3s2)cc1 ; c1ccc(-c2nc3ccc4ccc5ccc(N(c6ccccc6)c6ccc(-c7cccc8c7sc7ccccc78)cc6)cc5c4c3o2)cc1 ; c1ccc(-c2nc3ccc4ccc5ccc(N(c6ccccc6)c6ccc(-c7ccc8c(c7)sc7ccccc78)cc6)cc5c4c3o2)cc1 ; c1ccc(-c2nc3ccc4ccc5ccc(N(c6ccccc6)c6ccc(-c7ccc8sc9ccccc9c8c7)cc6)cc5c4c3o2)cc1]

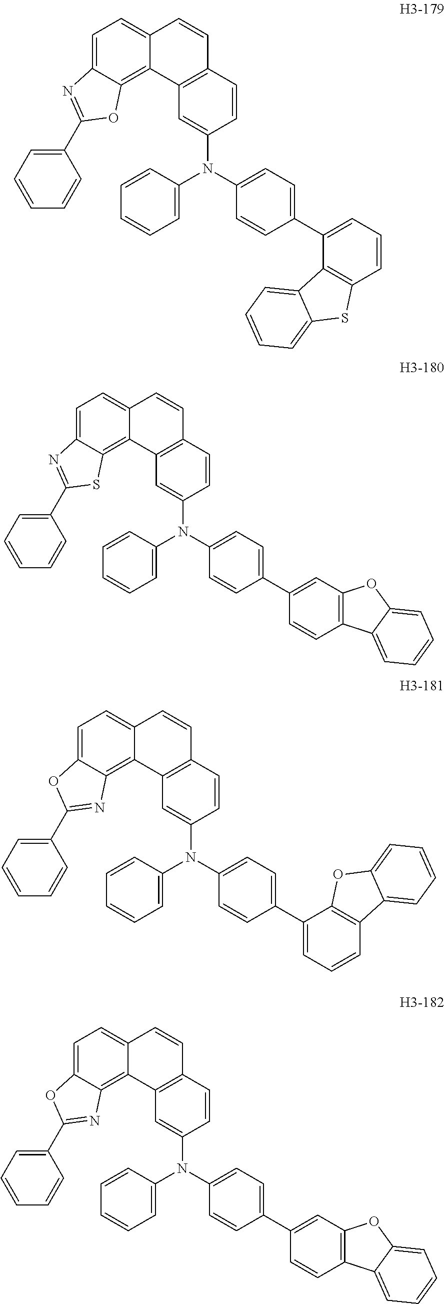 [SMILES: c1ccc(-c2nc3ccc4ccc5ccc(N(c6ccccc6)c6ccc(-c7cccc8sc9ccccc9c78)cc6)cc5c4c3o2)cc1 ; c1ccc(-c2nc3ccc4ccc5ccc(N(c6ccccc6)c6ccc(-c7ccc8c(c7)oc7ccccc78)cc6)cc5c4c3s2)cc1 ; c1ccc(-c2nc3c(ccc4ccc5ccc(N(c6ccccc6)c6ccc(-c7cccc8c7oc7ccccc78)cc6)cc5c43)o2)cc1 ; c1ccc(-c2nc3c(ccc4ccc5ccc(N(c6ccccc6)c6ccc(-c7ccc8c(c7)oc7ccccc78)cc6)cc5c43)o2)cc1]

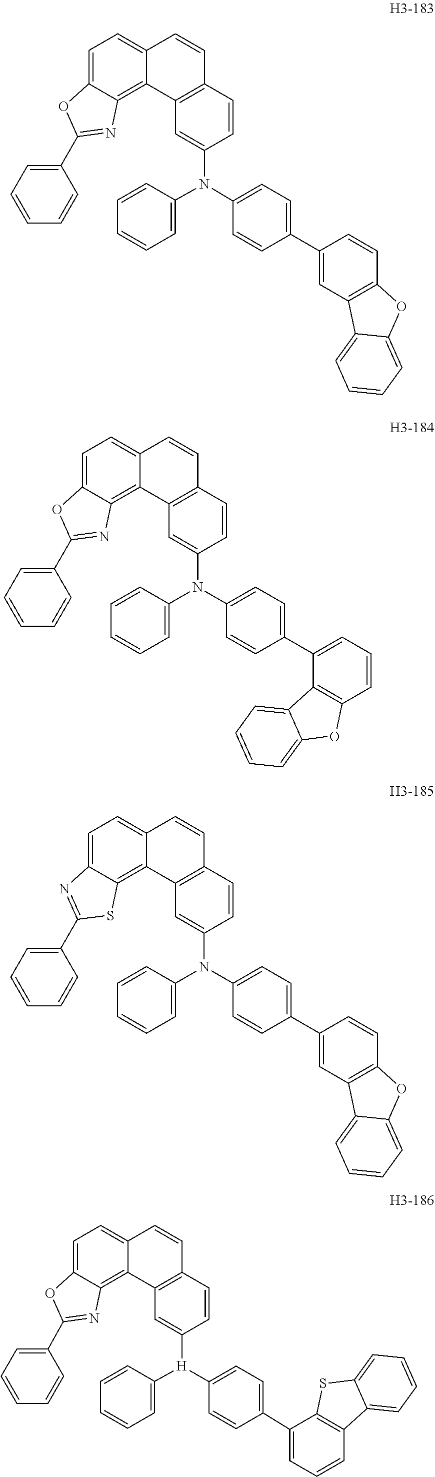

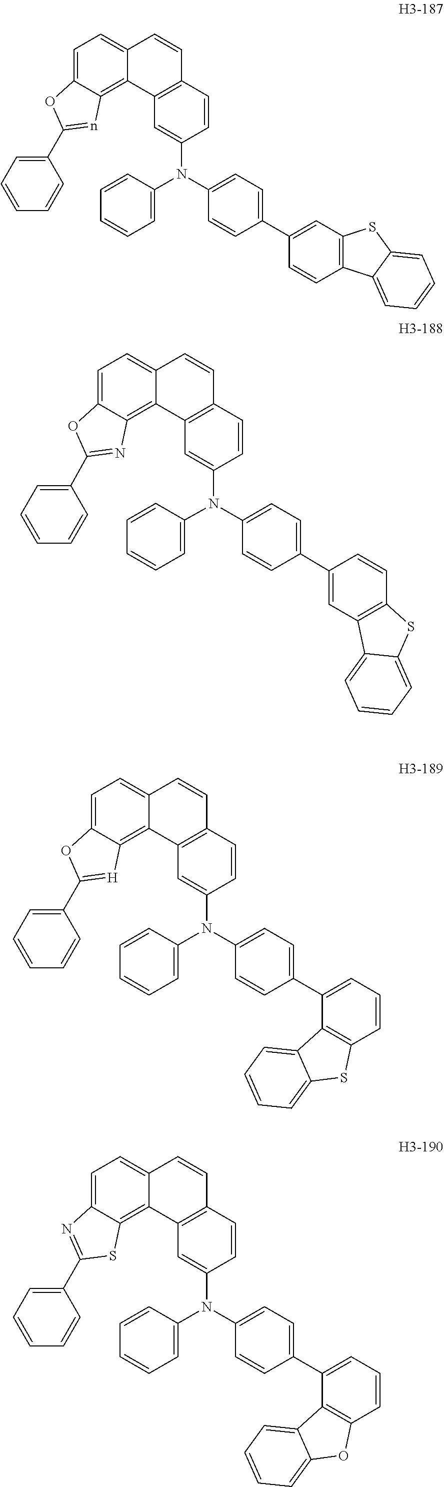

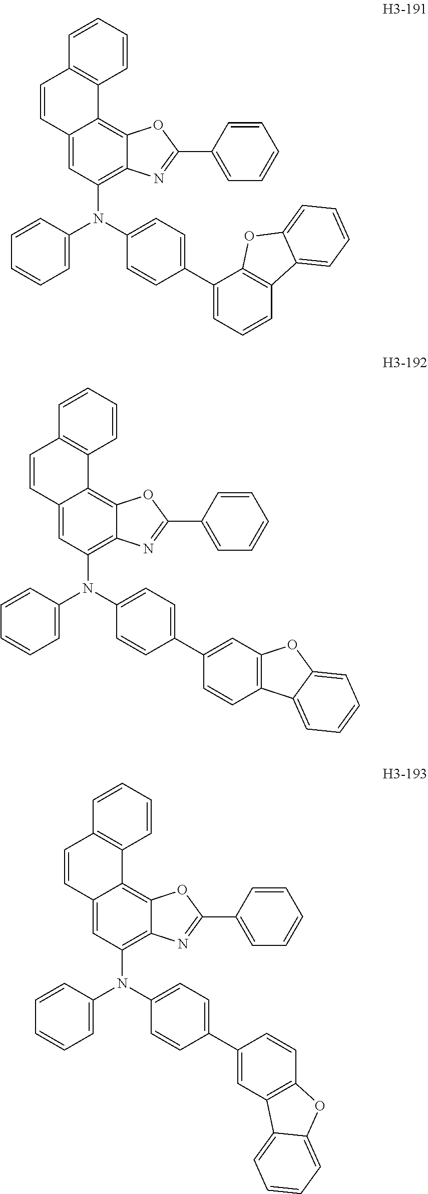 [SMILES: c1ccc(-c2nc3c(N(c4ccccc4)c4ccc(-c5cccc6c5oc5ccccc56)cc4)cc4ccc5ccccc5c4c3o2)cc1 ; c1ccc(-c2nc3c(N(c4ccccc4)c4ccc(-c5ccc6c(c5)oc5ccccc56)cc4)cc4ccc5ccccc5c4c3o2)cc1 ; c1ccc(-c2nc3c(N(c4ccccc4)c4ccc(-c5ccc6oc7ccccc7c6c5)cc4)cc4ccc5ccccc5c4c3o2)cc1]

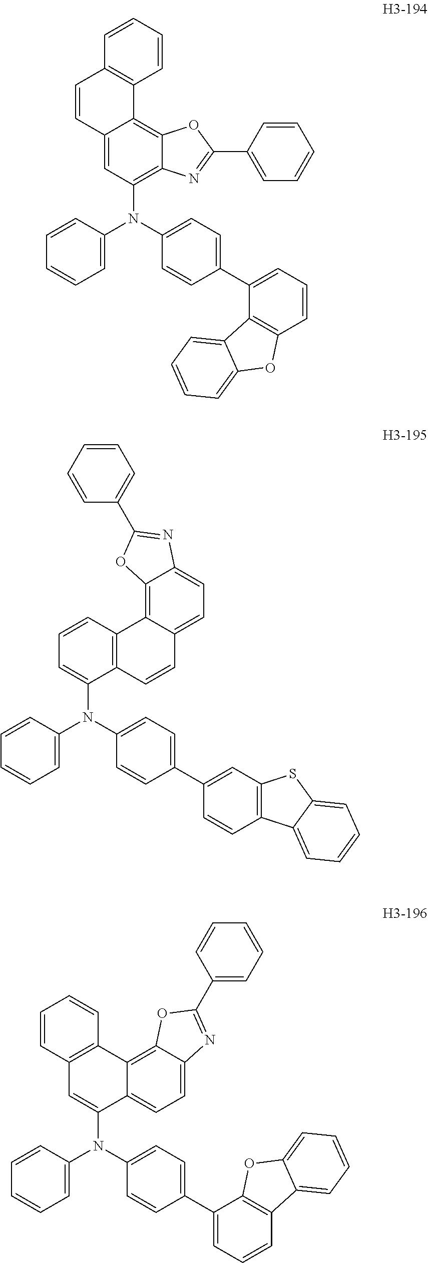 [SMILES: c1ccc(-c2nc3c(N(c4ccccc4)c4ccc(-c5cccc6oc7ccccc7c56)cc4)cc4ccc5ccccc5c4c3o2)cc1 ; c1ccc(-c2nc3ccc4ccc5c(N(c6ccccc6)c6ccc(-c7ccc8c(c7)sc7ccccc78)cc6)cccc5c4c3o2)cc1 ; c1ccc(-c2nc3ccc4c(N(c5ccccc5)c5ccc(-c6cccc7c6oc6ccccc67)cc5)cc5ccccc5c4c3o2)cc1]

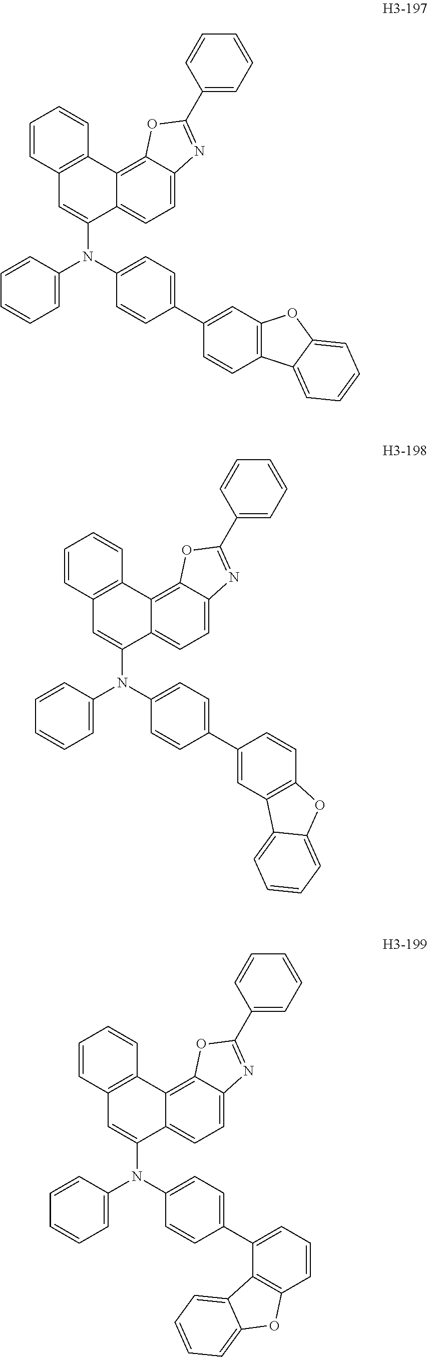 [SMILES: c1ccc(-c2nc3ccc4c(N(c5ccccc5)c5ccc(-c6ccc7c(c6)oc6ccccc67)cc5)cc5ccccc5c4c3o2)cc1 ; c1ccc(-c2nc3ccc4c(N(c5ccccc5)c5ccc(-c6ccc7oc8ccccc8c7c6)cc5)cc5ccccc5c4c3o2)cc1 ; c1ccc(-c2nc3ccc4c(N(c5ccccc5)c5ccc(-c6cccc7oc8ccccc8c67)cc5)cc5ccccc5c4c3o2)cc1]

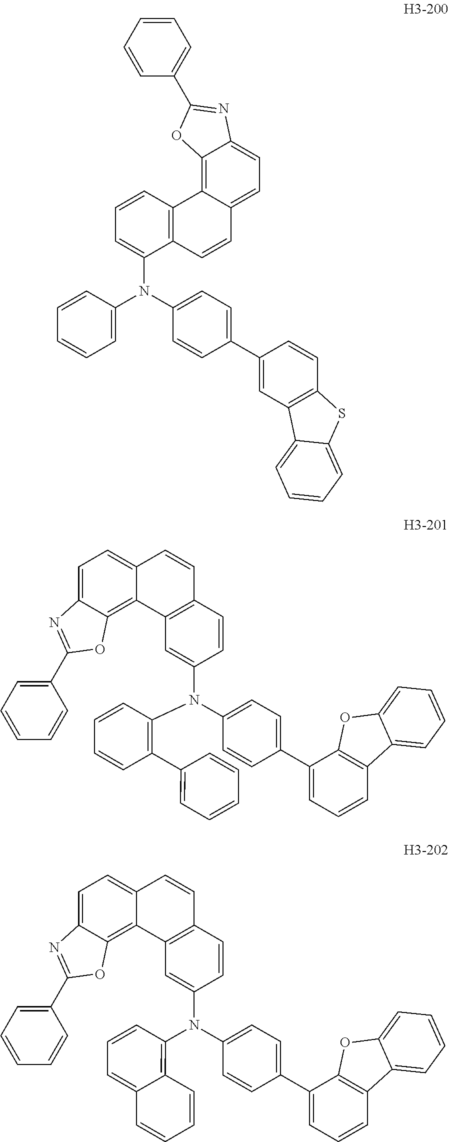 [SMILES: c1ccc(-c2nc3ccc4ccc5c(N(c6ccccc6)c6ccc(-c7ccc8sc9ccccc9c8c7)cc6)cccc5c4c3o2)cc1 ; c1ccc(-c2nc3ccc4ccc5ccc(N(c6ccc(-c7cccc8c7oc7ccccc78)cc6)c6ccccc6-c6ccccc6)cc5c4c3o2)cc1 ; c1ccc(-c2nc3ccc4ccc5ccc(N(c6ccc(-c7cccc8c7oc7ccccc78)cc6)c6cccc7ccccc67)cc5c4c3o2)cc1]

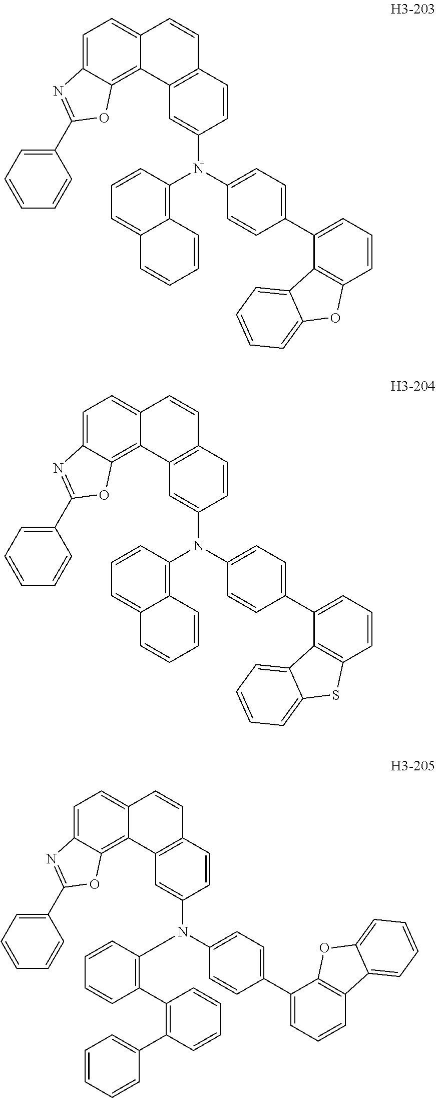

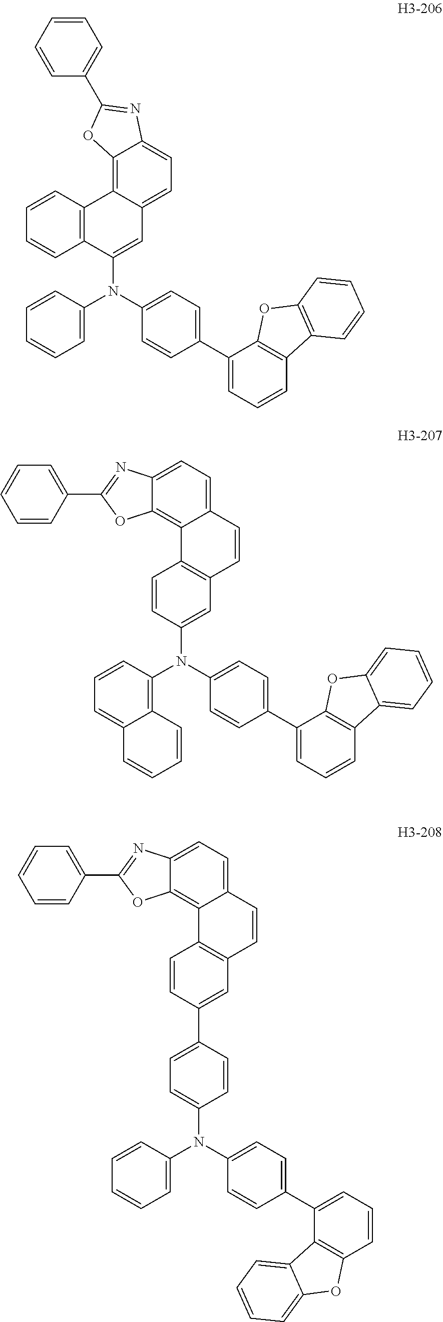 [SMILES: c1ccc(-c2nc3ccc4cc(N(c5ccccc5)c5ccc(-c6cccc7c6oc6ccccc67)cc5)c5ccccc5c4c3o2)cc1 ; c1ccc(-c2nc3ccc4ccc5cc(N(c6ccc(-c7cccc8c7oc7ccccc78)cc6)c6cccc7ccccc67)ccc5c4c3o2)cc1 ; c1ccc(-c2nc3ccc4ccc5cc(-c6ccc(N(c7ccccc7)c7ccc(-c8cccc9oc%10ccccc%10c89)cc7)cc6)ccc5c4c3o2)cc1]

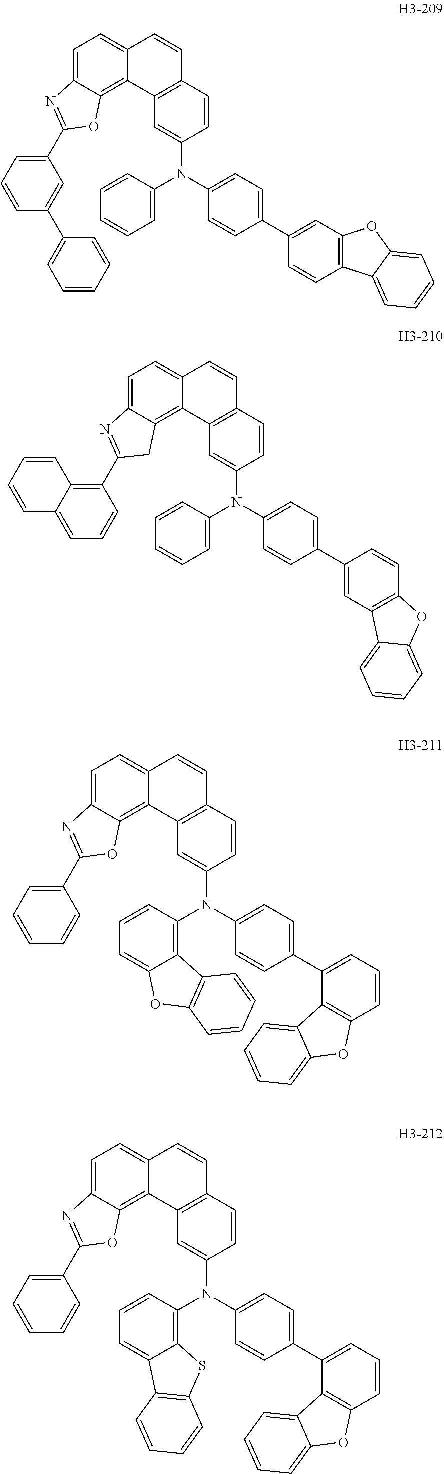 [SMILES: c1ccc(-c2cccc(-c3nc4ccc5ccc6ccc(N(c7ccccc7)c7ccc(-c8ccc9c(c8)oc8ccccc89)cc7)cc6c5c4o3)c2)cc1 ; c1ccc(N(c2ccc(-c3ccc4oc5ccccc5c4c3)cc2)c2ccc3ccc4ccc5c(c4c3c2)CC(c2cccc3ccccc23)=N5)cc1 ; c1ccc(-c2nc3ccc4ccc5ccc(N(c6ccc(-c7cccc8oc9ccccc9c78)cc6)c6cccc7oc8ccccc8c67)cc5c4c3o2)cc1 ; c1ccc(-c2nc3ccc4ccc5ccc(N(c6ccc(-c7cccc8oc9ccccc9c78)cc6)c6cccc7c6sc6ccccc67)cc5c4c3o2)cc1]

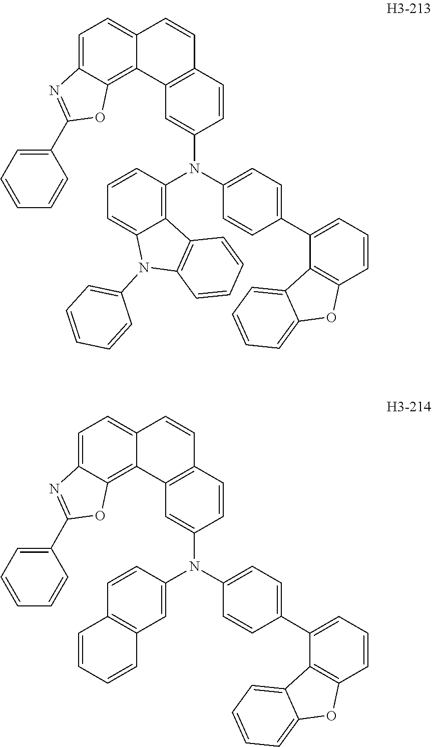 [SMILES: c1ccc(-c2nc3ccc4ccc5ccc(N(c6ccc(-c7cccc8oc9ccccc9c78)cc6)c6cccc7c6c6ccccc6n7-c6ccccc6)cc5c4c3o2)cc1 ; c1ccc(-c2nc3ccc4ccc5ccc(N(c6ccc(-c7cccc8oc9ccccc9c78)cc6)c6ccc7ccccc7c6)cc5c4c3o2)cc1]

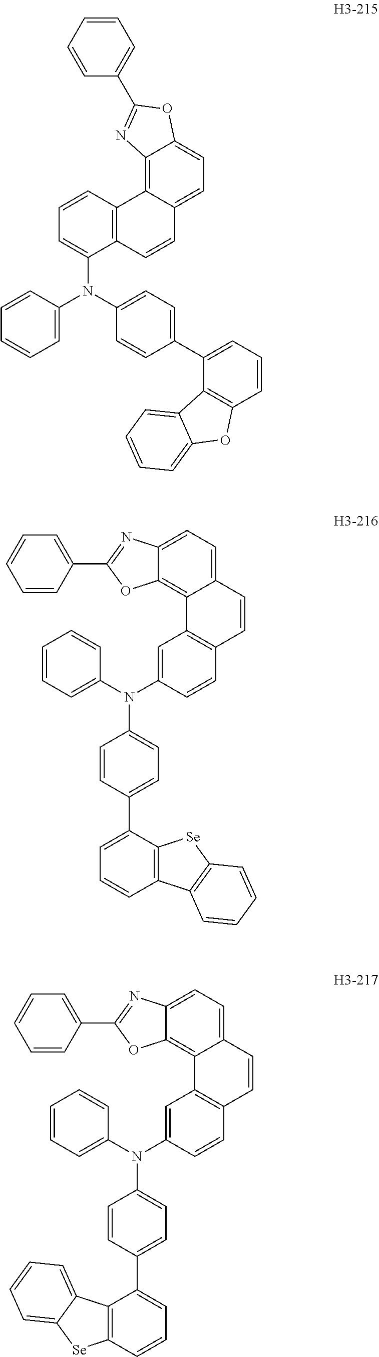 [SMILES: c1ccc(-c2nc3c(ccc4ccc5c(N(c6ccccc6)c6ccc(-c7cccc8oc9ccccc9c78)cc6)cccc5c43)o2)cc1 ; c1ccc(-c2nc3ccc4ccc5ccc(N(c6ccccc6)c6ccc(-c7cccc8c7[se]c7ccccc78)cc6)cc5c4c3o2)cc1 ; c1ccc(-c2nc3ccc4ccc5ccc(N(c6ccccc6)c6ccc(-c7cccc8[se]c9ccccc9c78)cc6)cc5c4c3o2)cc1]

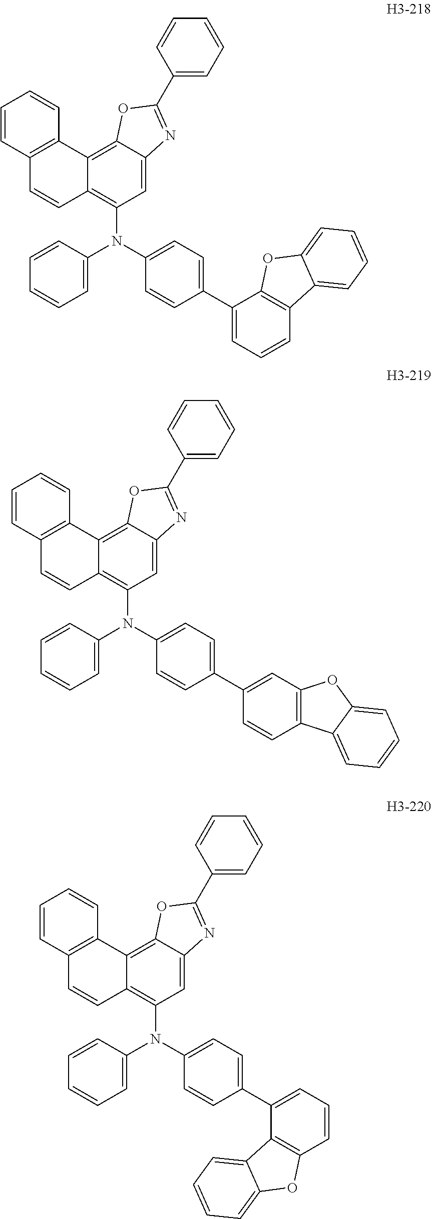 [SMILES: c1ccc(-c2nc3cc(N(c4ccccc4)c4ccc(-c5cccc6c5oc5ccccc56)cc4)c4ccc5ccccc5c4c3o2)cc1 ; c1ccc(-c2nc3cc(N(c4ccccc4)c4ccc(-c5ccc6c(c5)oc5ccccc56)cc4)c4ccc5ccccc5c4c3o2)cc1 ; c1ccc(-c2nc3cc(N(c4ccccc4)c4ccc(-c5cccc6oc7ccccc7c56)cc4)c4ccc5ccccc5c4c3o2)cc1]

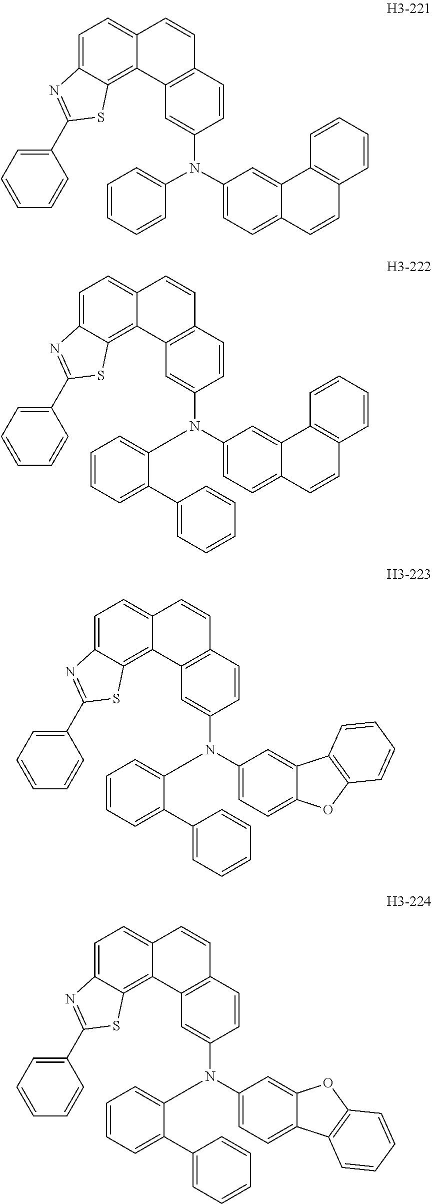 [SMILES: c1ccc(-c2nc3ccc4ccc5ccc(N(c6ccccc6)c6ccc7ccc8ccccc8c7c6)cc5c4c3s2)cc1 ; c1ccc(-c2nc3ccc4ccc5ccc(N(c6ccc7ccc8ccccc8c7c6)c6ccccc6-c6ccccc6)cc5c4c3s2)cc1 ; c1ccc(-c2nc3ccc4ccc5ccc(N(c6ccc7oc8ccccc8c7c6)c6ccccc6-c6ccccc6)cc5c4c3s2)cc1 ; c1ccc(-c2nc3ccc4ccc5ccc(N(c6ccc7c(c6)oc6ccccc67)c6ccccc6-c6ccccc6)cc5c4c3s2)cc1]

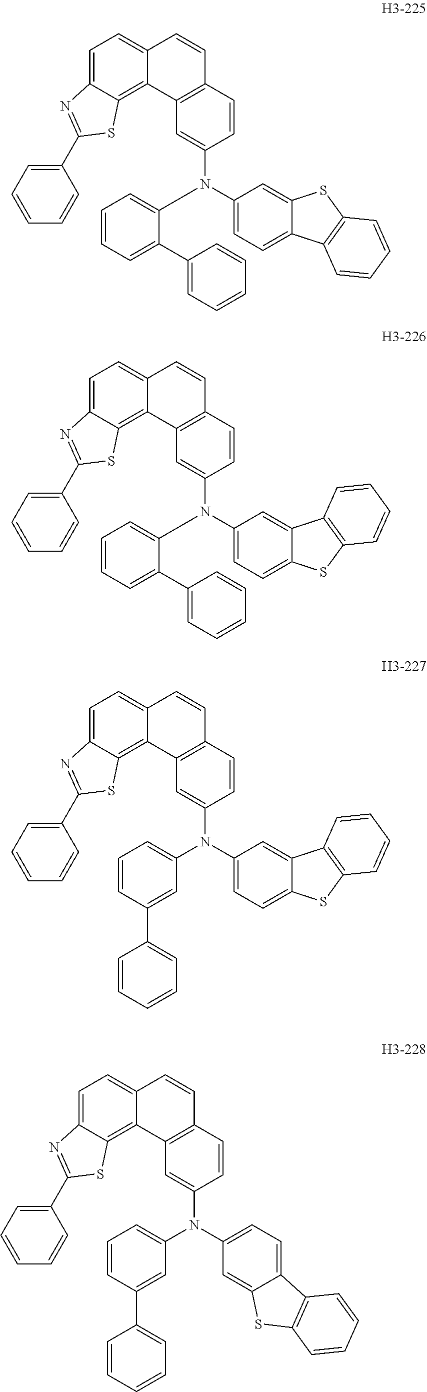 [SMILES: c1ccc(-c2nc3ccc4ccc5ccc(N(c6ccc7c(c6)sc6ccccc67)c6ccccc6-c6ccccc6)cc5c4c3s2)cc1 ; c1ccc(-c2nc3ccc4ccc5ccc(N(c6ccc7sc8ccccc8c7c6)c6ccccc6-c6ccccc6)cc5c4c3s2)cc1 ; c1ccc(-c2cccc(N(c3ccc4sc5ccccc5c4c3)c3ccc4ccc5ccc6nc(-c7ccccc7)sc6c5c4c3)c2)cc1 ; c1ccc(-c2cccc(N(c3ccc4c(c3)sc3ccccc34)c3ccc4ccc5ccc6nc(-c7ccccc7)sc6c5c4c3)c2)cc1]

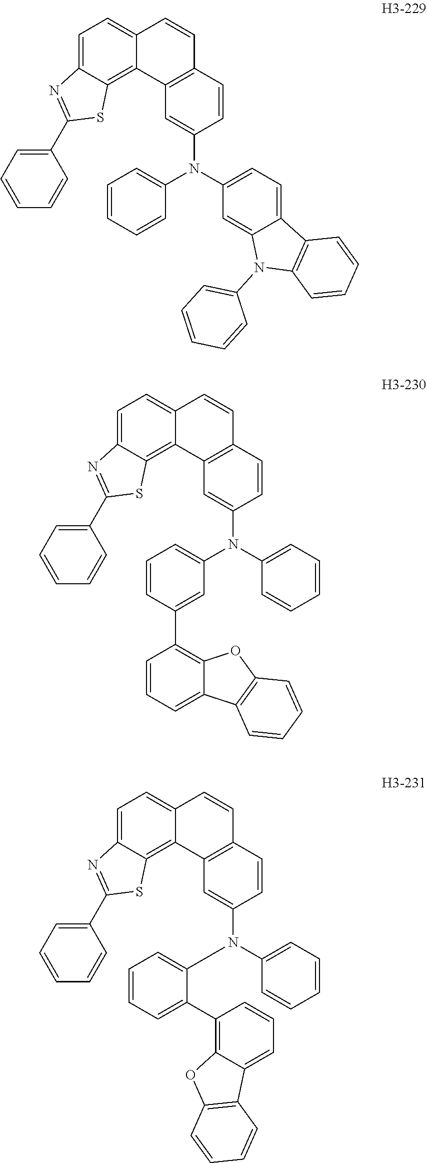 [SMILES: c1ccc(-c2nc3ccc4ccc5ccc(N(c6ccccc6)c6ccc7c8ccccc8n(-c8ccccc8)c7c6)cc5c4c3s2)cc1 ; c1ccc(-c2nc3ccc4ccc5ccc(N(c6ccccc6)c6cccc(-c7cccc8c7oc7ccccc78)c6)cc5c4c3s2)cc1 ; c1ccc(-c2nc3ccc4ccc5ccc(N(c6ccccc6)c6ccccc6-c6cccc7c6oc6ccccc67)cc5c4c3s2)cc1]

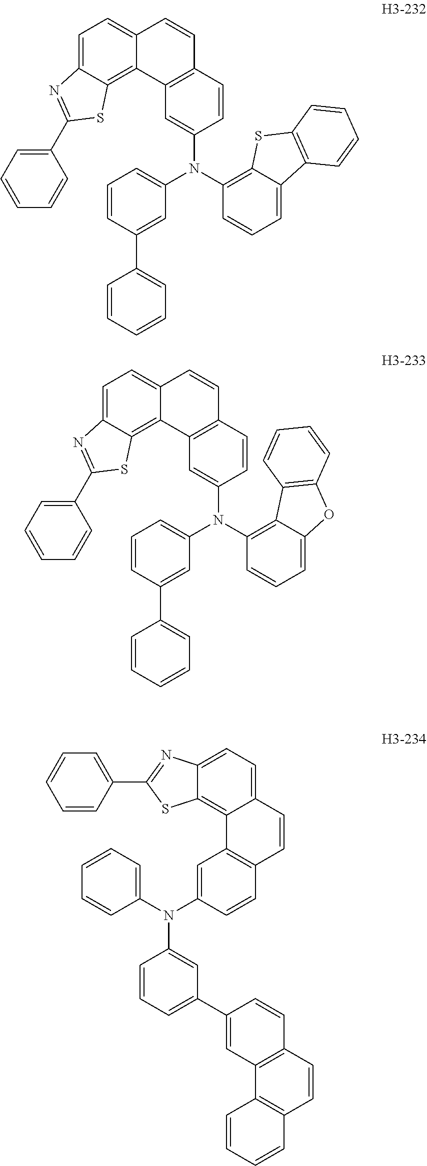

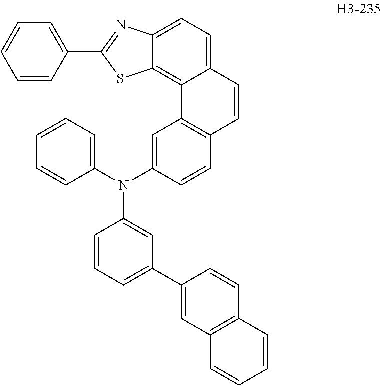 [SMILES: c1ccc(-c2nc3ccc4ccc5ccc(N(c6ccccc6)c6cccc(-c7ccc8ccccc8c7)c6)cc5c4c3s2)cc1]

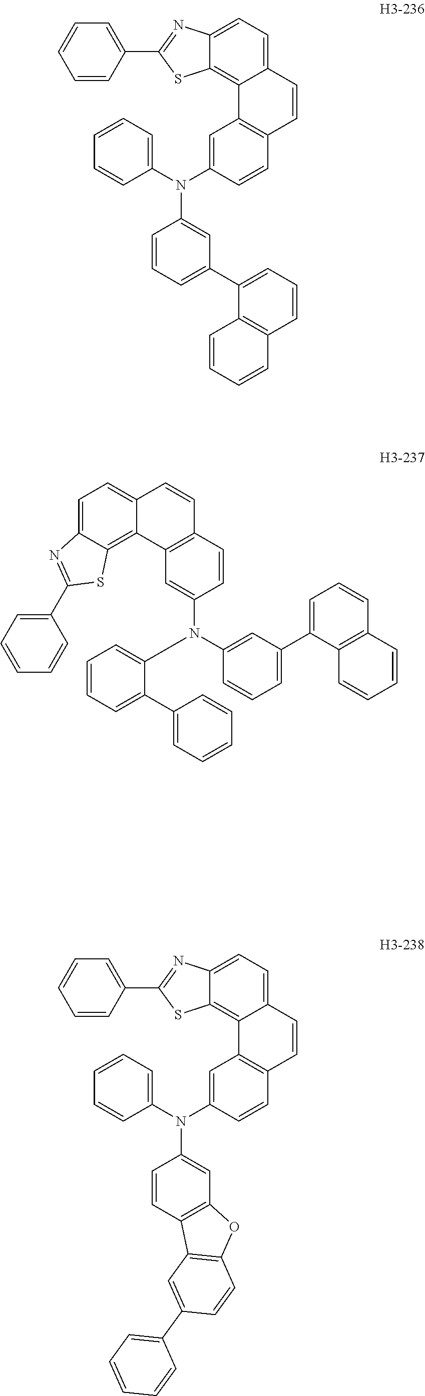 [SMILES: c1ccc(-c2nc3ccc4ccc5ccc(N(c6ccccc6)c6cccc(-c7cccc8ccccc78)c6)cc5c4c3s2)cc1 ; c1ccc(-c2nc3ccc4ccc5ccc(N(c6cccc(-c7cccc8ccccc78)c6)c6ccccc6-c6ccccc6)cc5c4c3s2)cc1 ; c1ccc(-c2ccc3oc4cc(N(c5ccccc5)c5ccc6ccc7ccc8nc(-c9ccccc9)sc8c7c6c5)ccc4c3c2)cc1]

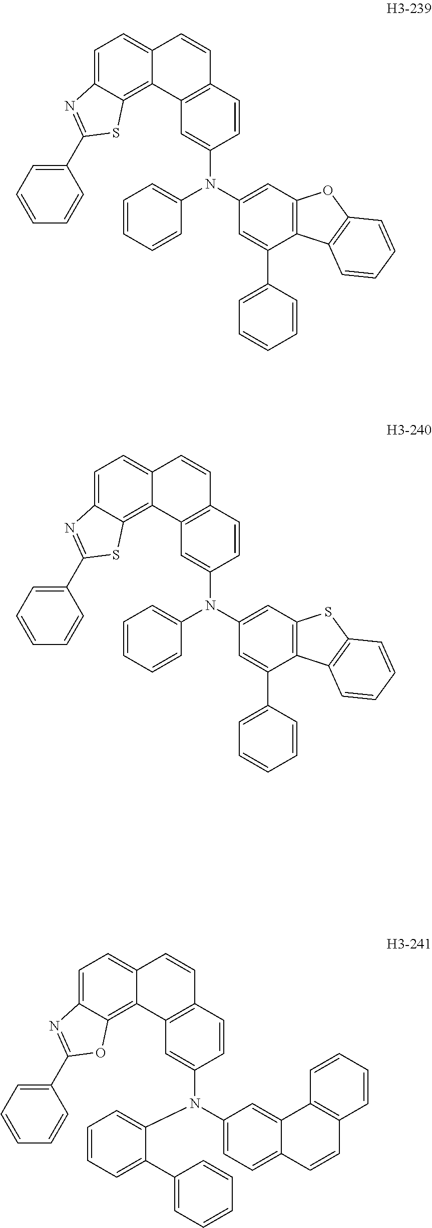 [SMILES: c1ccc(-c2nc3ccc4ccc5ccc(N(c6ccccc6)c6cc(-c7ccccc7)c7c(c6)oc6ccccc67)cc5c4c3s2)cc1 ; c1ccc(-c2nc3ccc4ccc5ccc(N(c6ccccc6)c6cc(-c7ccccc7)c7c(c6)sc6ccccc67)cc5c4c3s2)cc1 ; c1ccc(-c2nc3ccc4ccc5ccc(N(c6ccc7ccc8ccccc8c7c6)c6ccccc6-c6ccccc6)cc5c4c3o2)cc1]

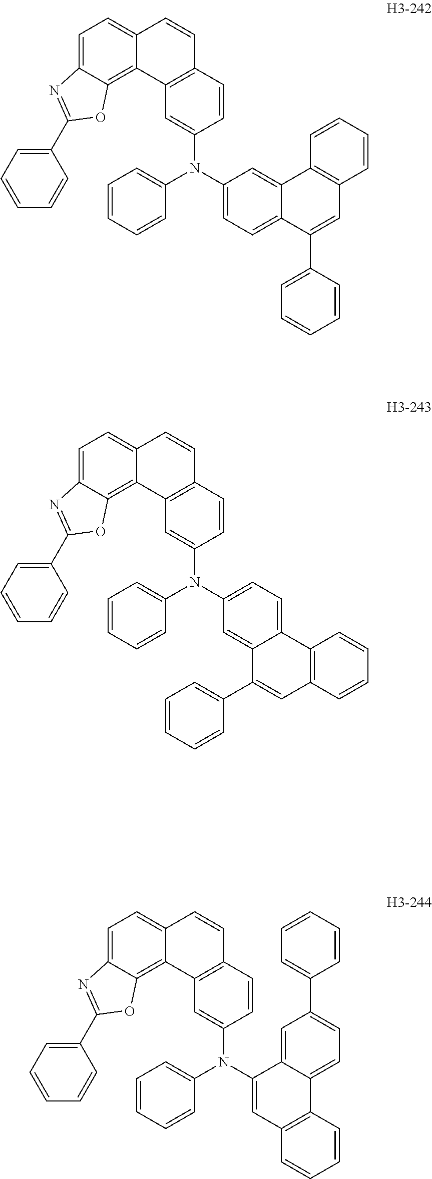 [SMILES: c1ccc(-c2nc3ccc4ccc5ccc(N(c6ccccc6)c6ccc7c(-c8ccccc8)cc8ccccc8c7c6)cc5c4c3o2)cc1 ; c1ccc(-c2nc3ccc4ccc5ccc(N(c6ccccc6)c6ccc7c(c6)c(-c6ccccc6)cc6ccccc67)cc5c4c3o2)cc1 ; c1ccc(-c2ccc3c(c2)c(N(c2ccccc2)c2ccc4ccc5ccc6nc(-c7ccccc7)oc6c5c4c2)cc2ccccc23)cc1]

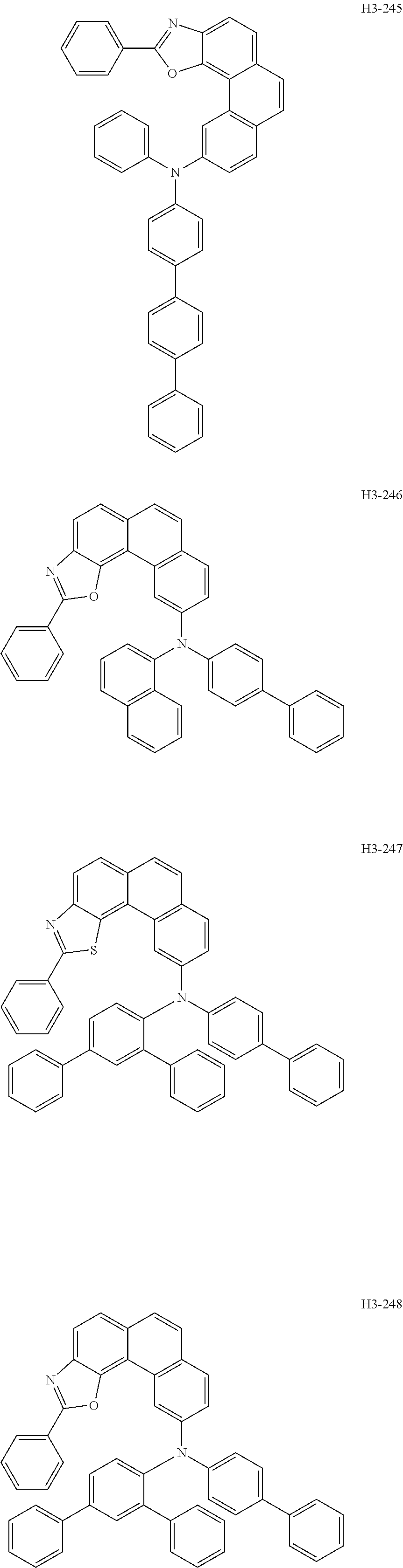 [SMILES: c1ccc(-c2ccc(-c3ccc(N(c4ccccc4)c4ccc5ccc6ccc7nc(-c8ccccc8)oc7c6c5c4)cc3)cc2)cc1 ; c1ccc(-c2ccc(N(c3ccc4ccc5ccc6nc(-c7ccccc7)oc6c5c4c3)c3cccc4ccccc34)cc2)cc1 ; c1ccc(-c2ccc(N(c3ccc4ccc5ccc6nc(-c7ccccc7)sc6c5c4c3)c3ccc(-c4ccccc4)cc3-c3ccccc3)cc2)cc1 ; c1ccc(-c2ccc(N(c3ccc4ccc5ccc6nc(-c7ccccc7)oc6c5c4c3)c3ccc(-c4ccccc4)cc3-c3ccccc3)cc2)cc1]

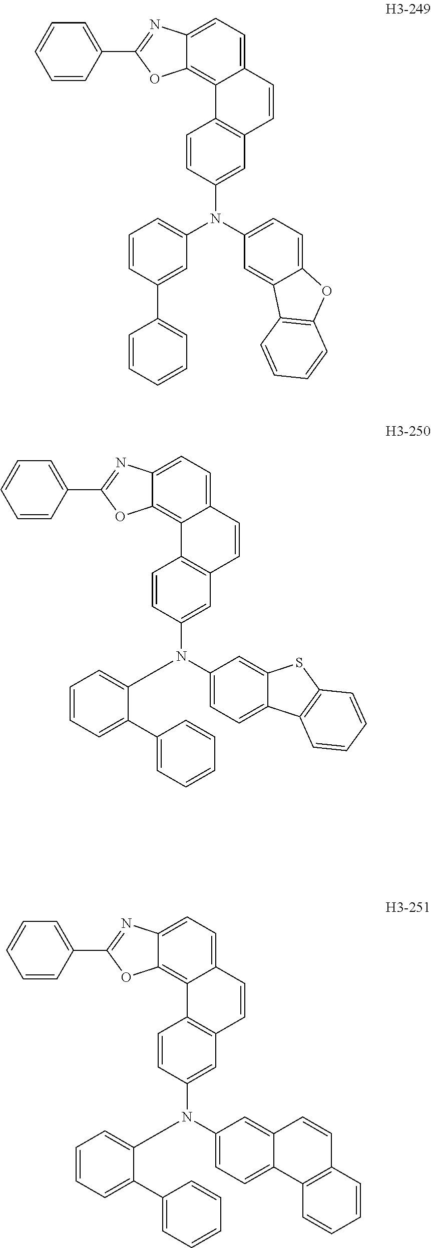 [SMILES: c1ccc(-c2cccc(N(c3ccc4c(ccc5ccc6nc(-c7ccccc7)oc6c54)c3)c3ccc4oc5ccccc5c4c3)c2)cc1 ; c1ccc(-c2nc3ccc4ccc5cc(N(c6ccc7c(c6)sc6ccccc67)c6ccccc6-c6ccccc6)ccc5c4c3o2)cc1 ; c1ccc(-c2nc3ccc4ccc5cc(N(c6ccc7c(ccc8ccccc87)c6)c6ccccc6-c6ccccc6)ccc5c4c3o2)cc1]

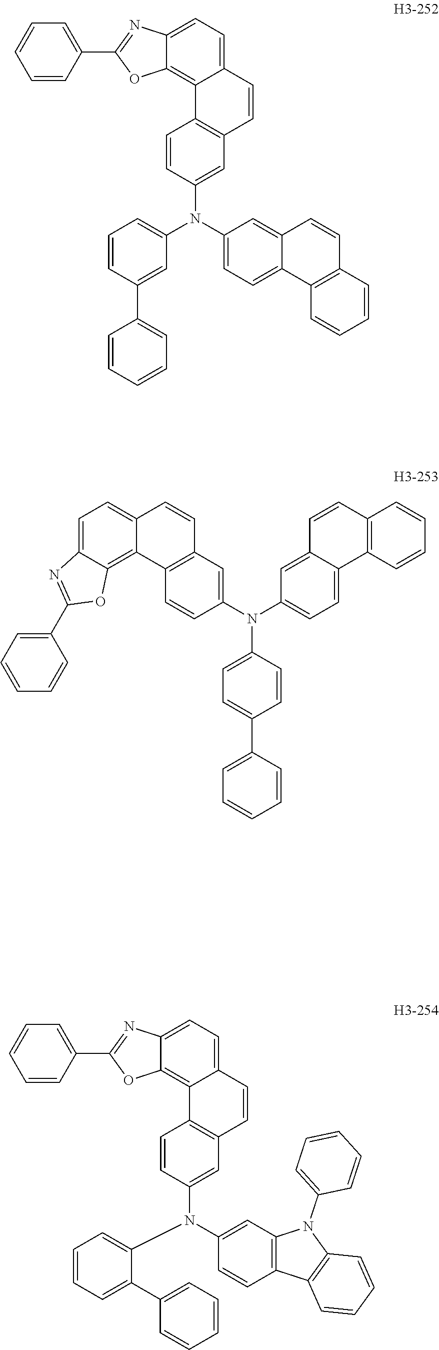 [SMILES: c1ccc(-c2cccc(N(c3ccc4c(ccc5ccccc54)c3)c3ccc4c(ccc5ccc6nc(-c7ccccc7)oc6c54)c3)c2)cc1 ; c1ccc(-c2ccc(N(c3ccc4c(ccc5ccccc54)c3)c3ccc4c(ccc5ccc6nc(-c7ccccc7)oc6c54)c3)cc2)cc1 ; c1ccc(-c2nc3ccc4ccc5cc(N(c6ccc7c8ccccc8n(-c8ccccc8)c7c6)c6ccccc6-c6ccccc6)ccc5c4c3o2)cc1]

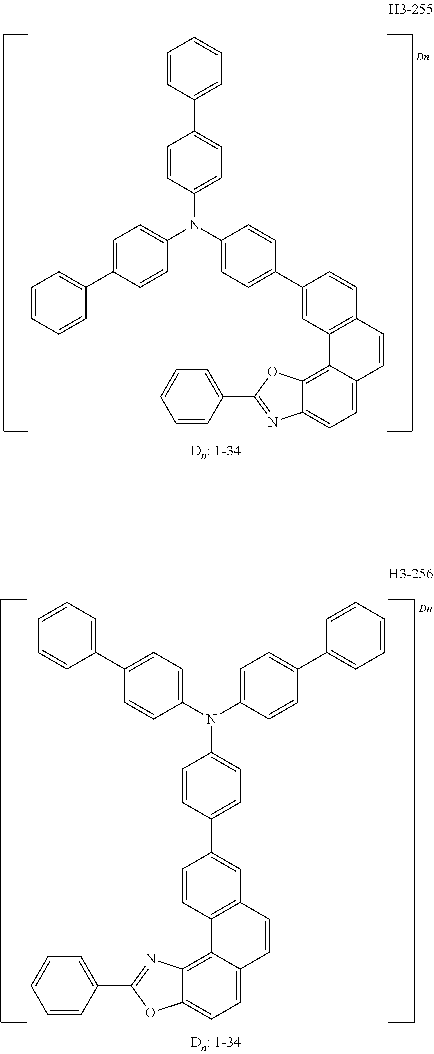

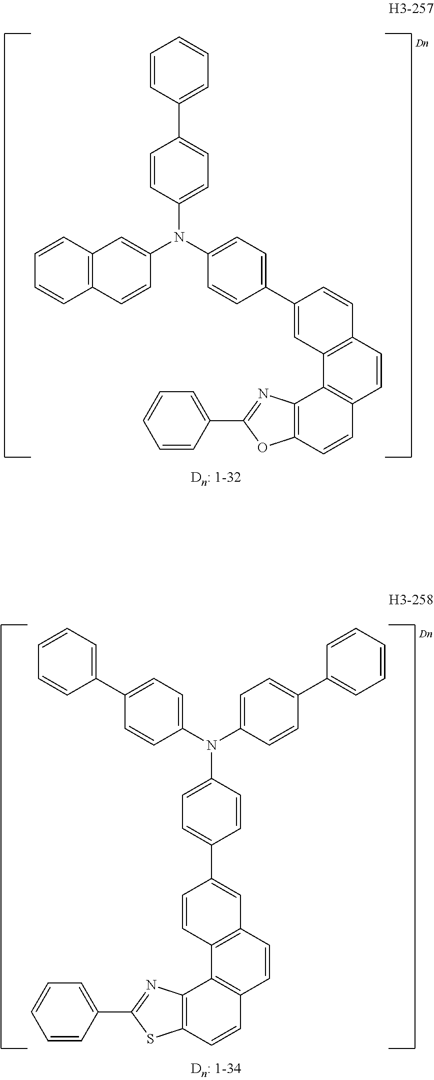

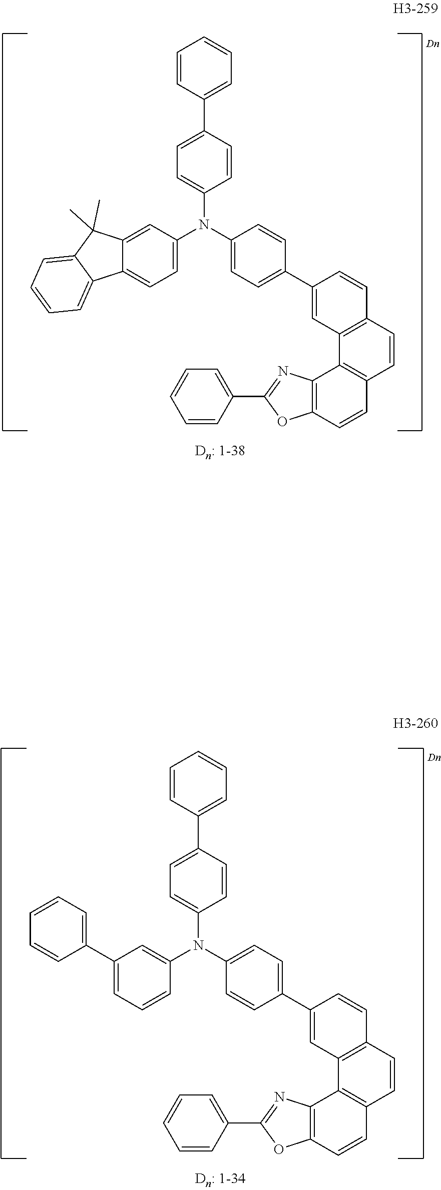

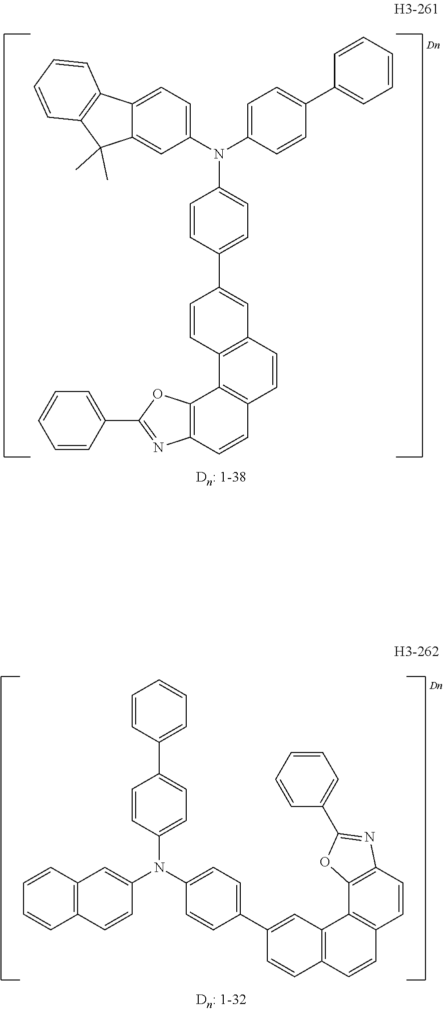

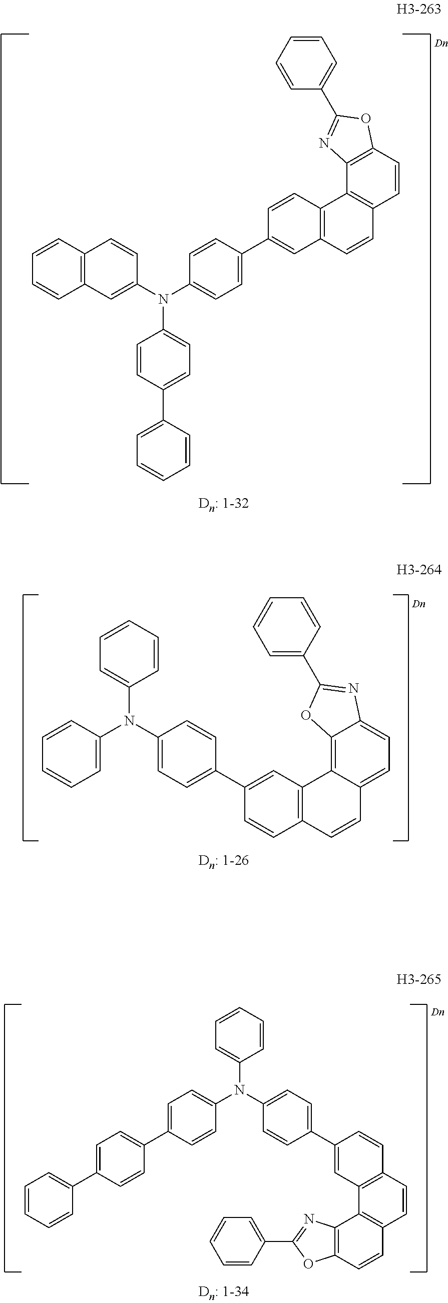

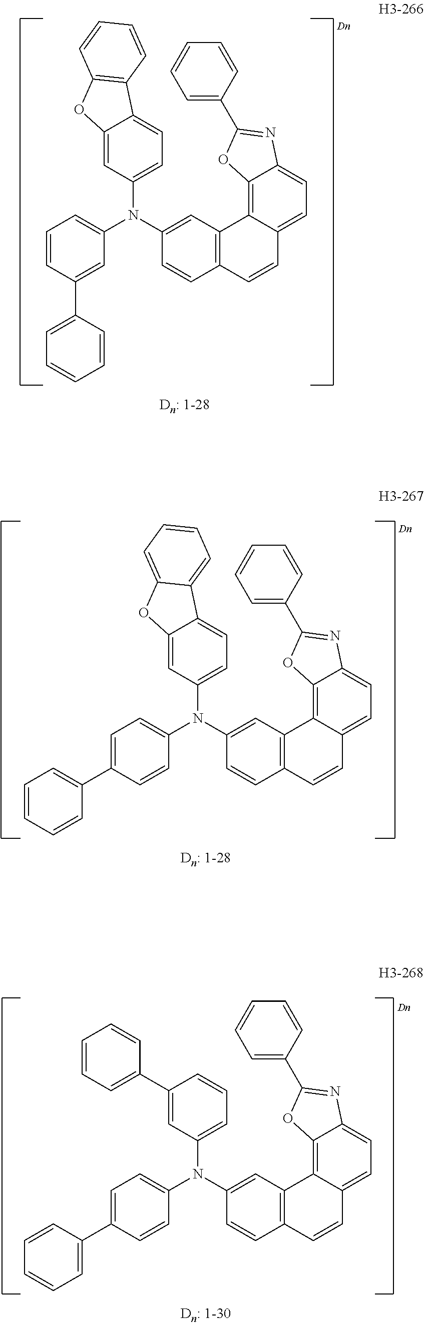

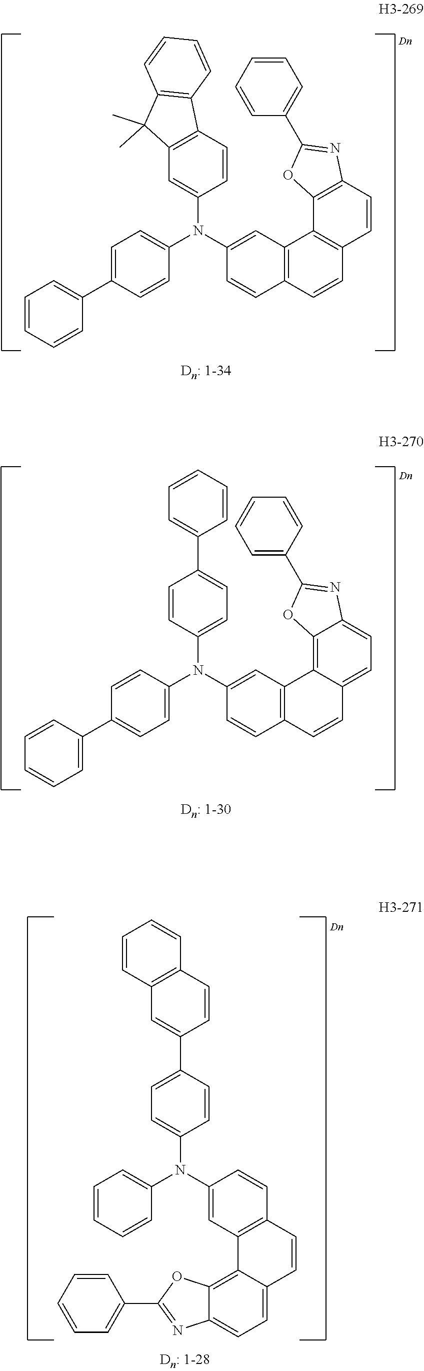

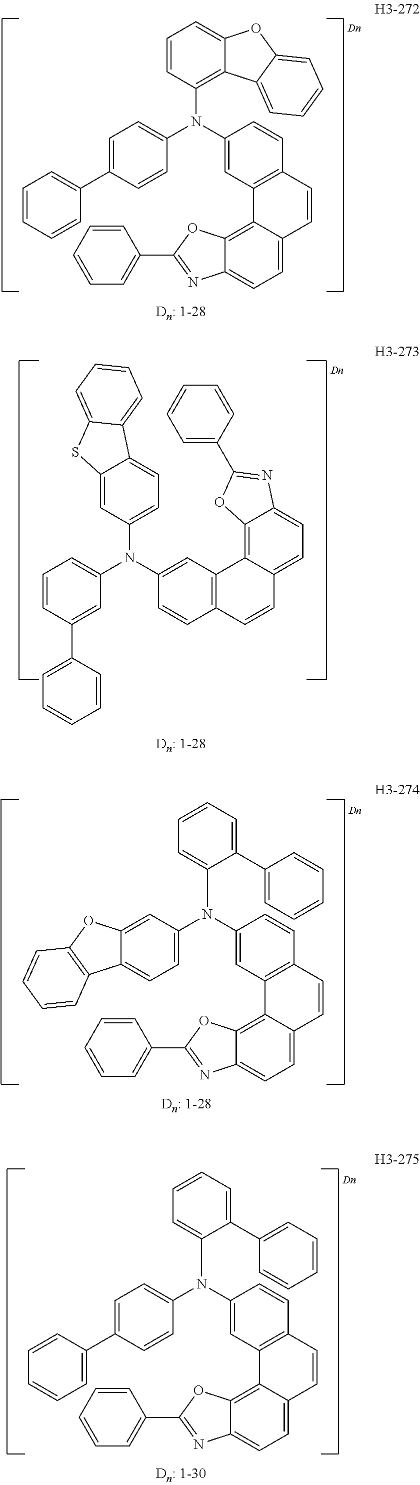

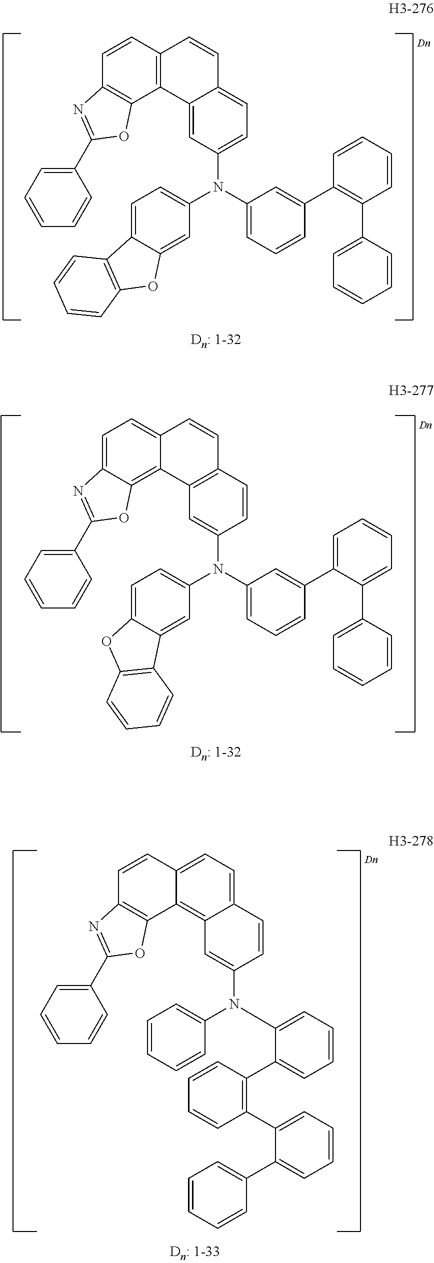

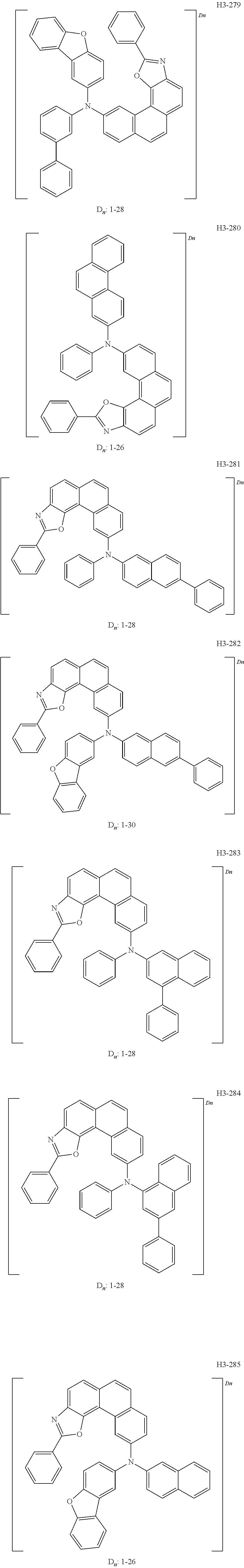

In each of Compounds H3-255 to H3-285, Dₙ means that 1 to 26, 1 to 28, 1 to 30, 1 to 32, 1 to 33, 1 to 34, or 1 to 38 hydrogens are replaced with deuterium.

The compounds represented by Formulas 1, 4, and 5 may be produced by synthetic methods known to one skilled in the art. For example, the compounds represented by Formulas 1 and 4 may be synthesized by referring to the following Reaction Schemes 1 and 2, but are not limited thereto. In addition, the compound represented by Formula 5 may be produced by referring to Korean Patent Application Laid-Open Nos. 2017-0022865 (published on Mar. 2, 2017) and 2018-0099487 (published on Sep. 5, 2018), etc., but is not limited thereto.

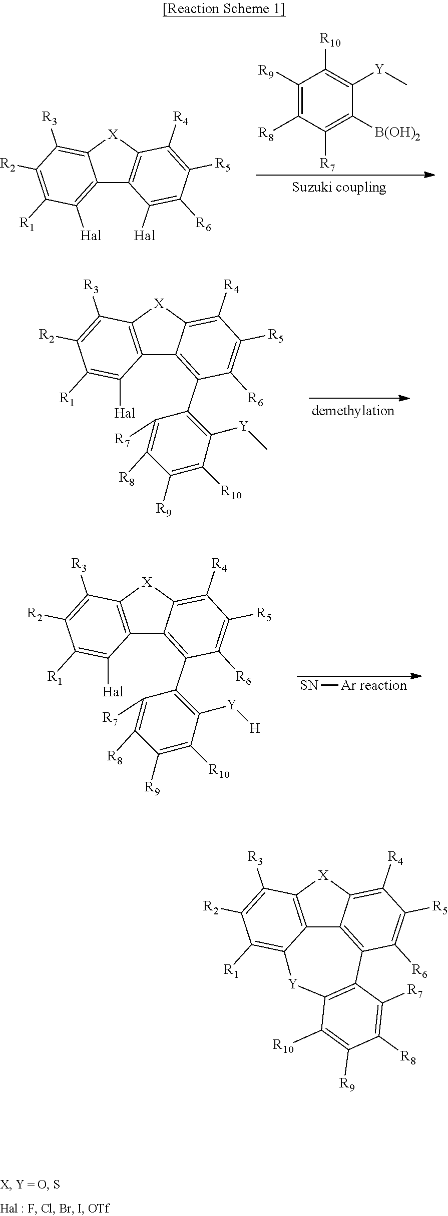

In Reaction Scheme 1, R₁ to R₁₀ are as defined in Formula 1, respectively.

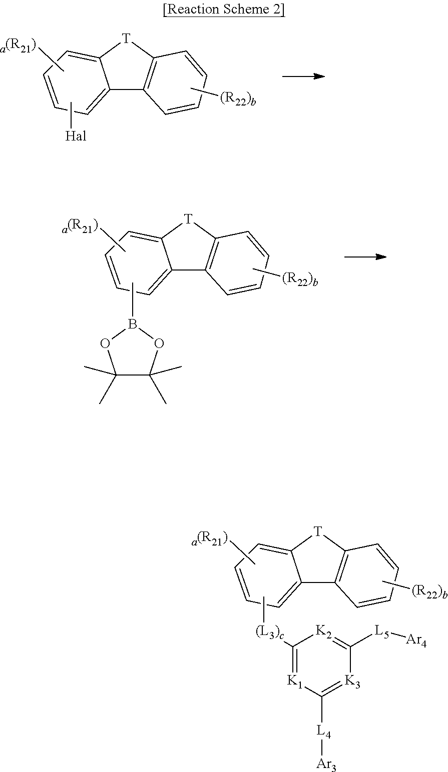

In Reaction Scheme 2, R₂₁, R₂₂, T, L₃ to L₅, K₁ to K₃, Ar₃, Ar₄, a, b, and c are as defined in Formula 4, respectively, and Hal refers to halogen.

Although illustrative synthesis examples of the compounds represented by Formulas 1 and 4 are described above, one skilled in the art will be able to easily understand that all of them are based on a Buchwald-Hartwig cross-coupling reaction, an N-arylation reaction, an H-mont-mediated etherification reaction, a Miyaura borylation reaction, a Suzuki cross-coupling reaction, an intramolecular acid-induced cyclization reaction, a Pd(II)-catalyzed oxidative cyclization reaction, a Grignard reaction, a Heck reaction, a cyclic dehydration reaction, an SN₁ substitution reaction, an SN₂ substitution reaction, a phosphine-mediated reductive cyclization reaction, Wittig reaction, etc., and the reaction above proceeds even when substituents which are defined in Formulas 1 and 4, but are not specified in the specific synthesis example, are bonded.

The compound represented by Formula 1 may be comprised in one or more layers constituting the organic electroluminescent device, for example, at least one layer selected from the group consisting of a hole injection layer, a hole transport layer, a hole auxiliary layer, a light-emitting auxiliary layer, a light-emitting layer, an electron transport layer, an electron buffer layer, an electron injection layer, an interlayer, a hole blocking layer, and an electron blocking layer. Each of the layers may be further configured as a plurality of layers. In addition, the compound represented by Formula 1 may be comprised in a light-emitting layer(s) and/or a hole transport layer(s), but is not limited thereto. The compound represented by Formula 1 may be comprised in a light-emitting layer(s) as a host, more specifically as a phosphorescent red host.

The organic electroluminescent device of the present disclosure comprises a first electrode; a second electrode; and at least one organic layer between the first electrode and the second electrode. One of the first and second electrodes may be an anode, and the other may be a cathode. The organic layer may comprise at least one light-emitting layer, and may further comprise at least one layer selected from the group consisting of a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer.

The first electrode and the second electrode may each be formed with a transmissive conductive material, a transflective conductive material, or a reflective conductive material. The organic electroluminescent device may be a top emission type, a bottom emission type, or both-sides emission type depending on the type of the material forming the first electrode and the second electrode. In addition, the hole injection layer may be further doped with a p-dopant, and the electron injection layer may be further doped with an n-dopant.

An organic electroluminescent device according to one embodiment of the present disclosure may be an organic electroluminescent device having a tandem structure. In the case of the tandem organic electroluminescent device according to one embodiment, a single light-emitting unit (light-emitting part) may be formed in a structure in which two or more units are connected by a charge generation layer. The organic electroluminescent device may include a plurality of two or more light-emitting units, for example, a plurality of three or more light-emitting units, having first and second electrodes opposed to each other on a substrate and a light-emitting layer stacked between the first and second electrodes and emits light in a specific wavelength range. It may include a plurality of light-emitting units, and each of the light-emitting units may include a hole transport zone, a light-emitting layer, and an electron transport zone, and the hole transport zone may include a hole injection layer and a hole transport layer, the electron transport zone may include an electron transport layer and an electron injection layer. According to one embodiment of the present disclosure, three or more light-emitting layers may be included in the light emitting unit. A plurality of light-emitting units may emit the same color or different colors. In addition, one light-emitting unit may include one or more light-emitting layers, the plurality of light-emitting layers may be light-emitting layers of the same or different colors. It may include one or more charge-generation layers located between each light-emitting unit. The charge generation layer refers to the layer in which holes and electrons are generated when voltage is applied. When there are three or more light-emitting units, a charge generation layer may be located between each light-emitting unit. The plurality of charge generation layers may be the same as or different from each other. By disposing the charge generation layer between light-emitting units, current efficiency is increased in each light-emitting unit and charges can be smoothly distributed. Specifically, the charge generation layer is provided between two adjacent stacks and can serve to drive a tandem organic electroluminescent device using only a pair of anode and cathode without a separate internal electrode located between the stacks.

The charge generation layer may be composed of an N-type charge generation layer and a P-type charge generation layer, and the N-type charge generation layer may be doped with an alkali metal, an alkaline earth metal, or a compound of an alkali metal and an alkaline earth metal, The alkali metal may include one selected from the group consisting of Li, Na, K, Rb, Cs, Fr, Yb, and combinations thereof, and the alkaline earth metal may include one selected from the group consisting of Be, Mg, Ca, Sr, Ba, Ra, and combinations thereof. The P-type charge generation layer may be made of a metal or an organic material doped with a P-type dopant. For example, the metal may be made of one or two or more alloys selected from the group consisting of Al, Cu, Fe, Pb, Zn, Au, Pt, W, In, Mo, Ni, and Ti. In addition, commonly used materials may be used as the P-type dopant and host materials used in the P-type doped organic material.

An organic electroluminescent device according to one embodiment may further comprise one or more dopants in the light-emitting layer. The dopant comprised in the organic electroluminescent device of the present disclosure may be at least one phosphorescent or fluorescent dopant, and preferably a phosphorescent dopant. The phosphorescent dopant material is not particularly limited, but may be a complex compound of metal selected from the group consisting of iridium (Ir), osmium (Os), copper (Cu), and platinum (Pt). Preferably, the complex compound of metal may be an ortho-metallated complex compound of metal selected from the group consisting of iridium (Ir), osmium (Os), copper (Cu), and platinum (Pt), and more preferably, it may be an ortho-metallated iridium complex compound.

The dopant comprised in the organic electroluminescent device of the present disclosure may be a compound represented by the following Formula 101, but is not limited thereto.

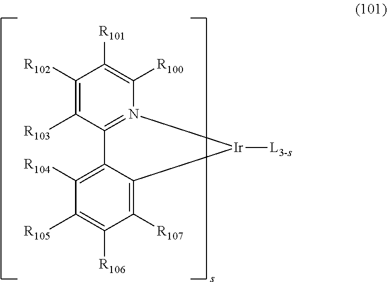

In Formula 101,
L is selected from the following structures 1 to 3:

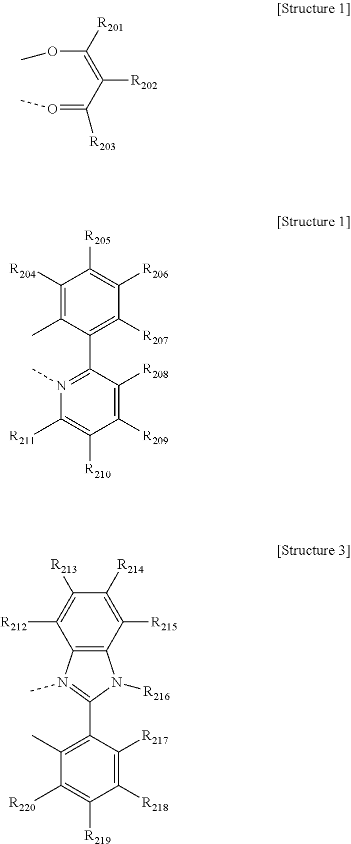

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent(s) to form a ring(s), e.g., a substituted or unsubstituted, quinoline, benzofuropyridine, benzothienopyridine, indenopyridine, benzofuroquinoline, benzothienoquinoline, or indenoquinoline, together with pyridine;
R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent(s) to form a substituted or unsubstituted ring(s), e.g., a substituted or unsubstituted, naphthalene, fluorene, dibenzothiophene, dibenzofuran, indenopyridine, benzofuropyridine, or benzothienopyridine, together with benzene;
R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent(s) to form a substituted or unsubstituted ring(s); and
s represents an integer of 1 to 3.

The specific examples of the dopant compound are as follows, but are not limited thereto.

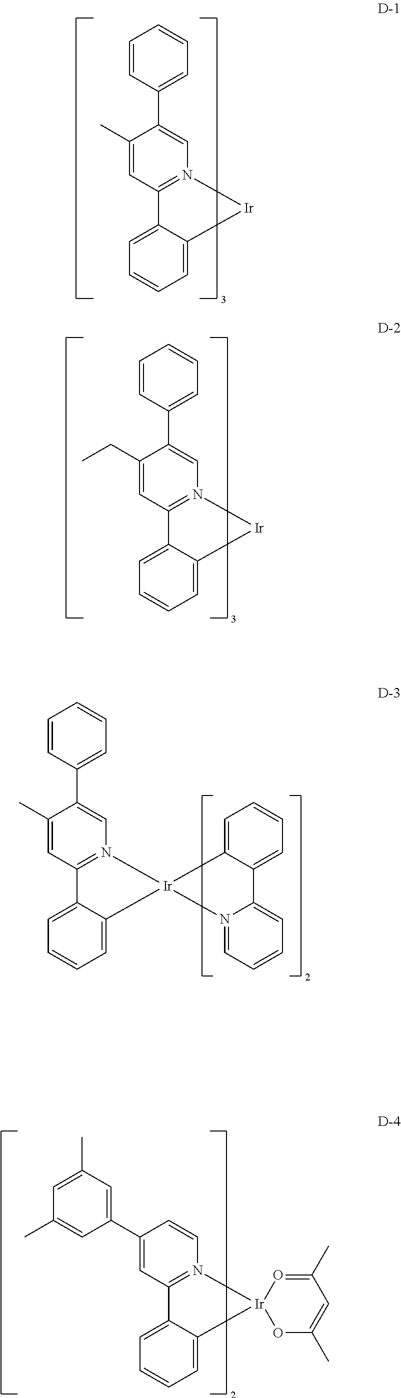

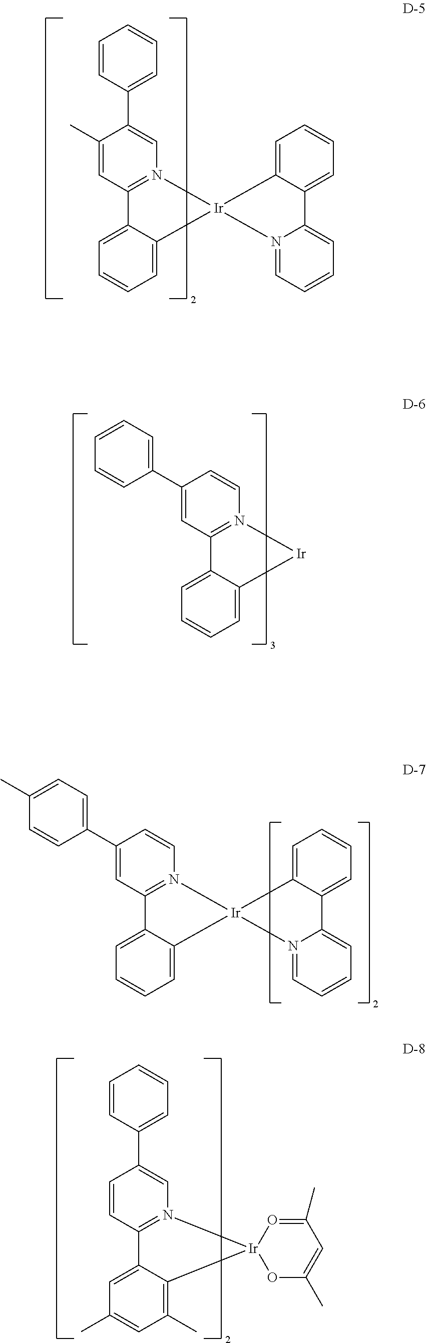

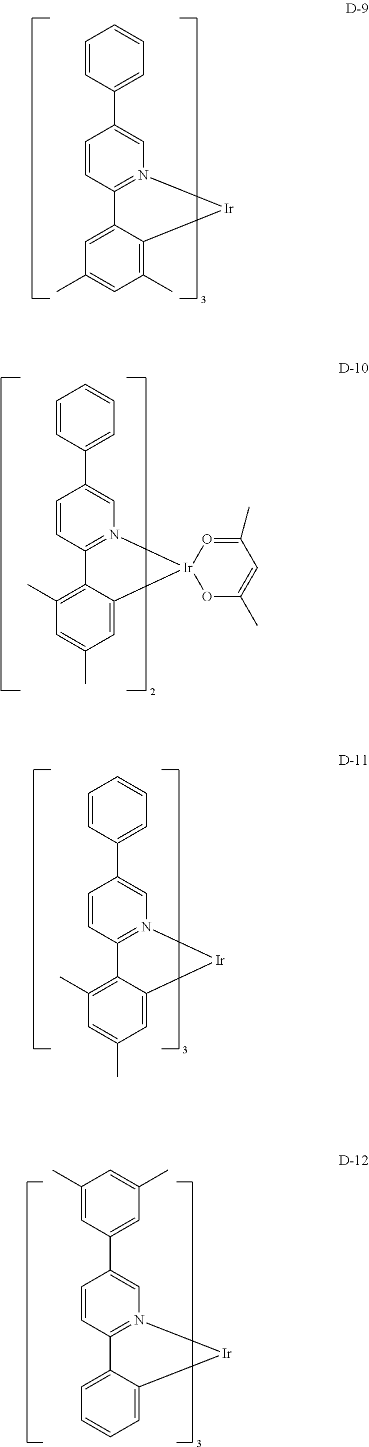

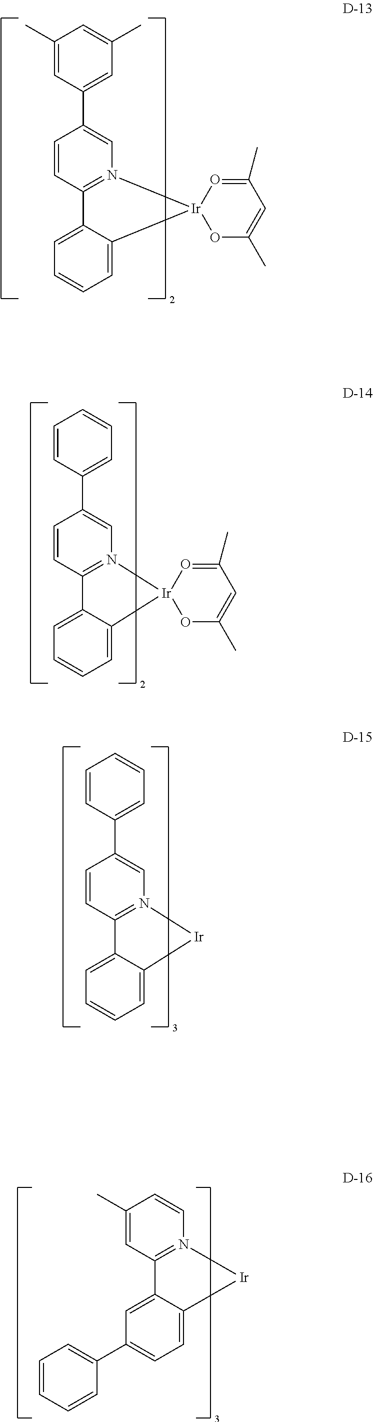

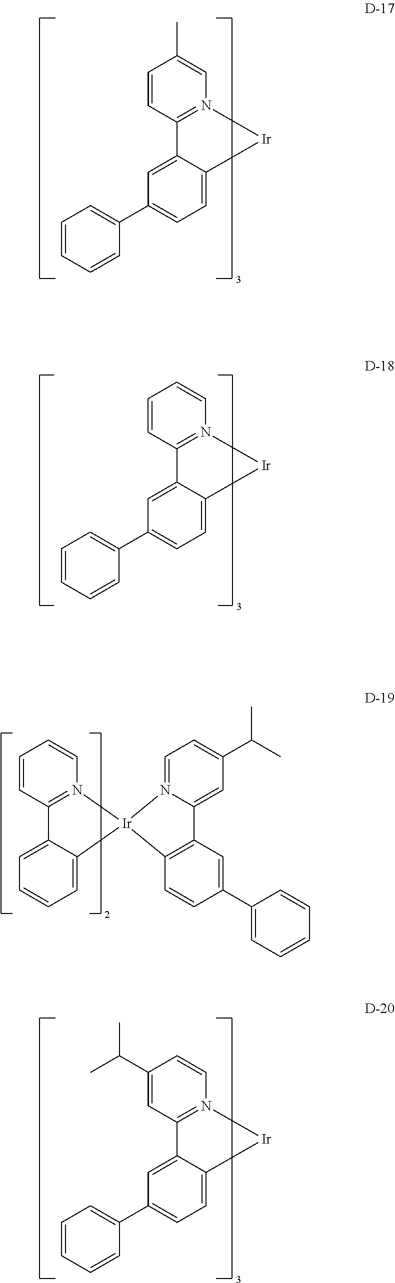

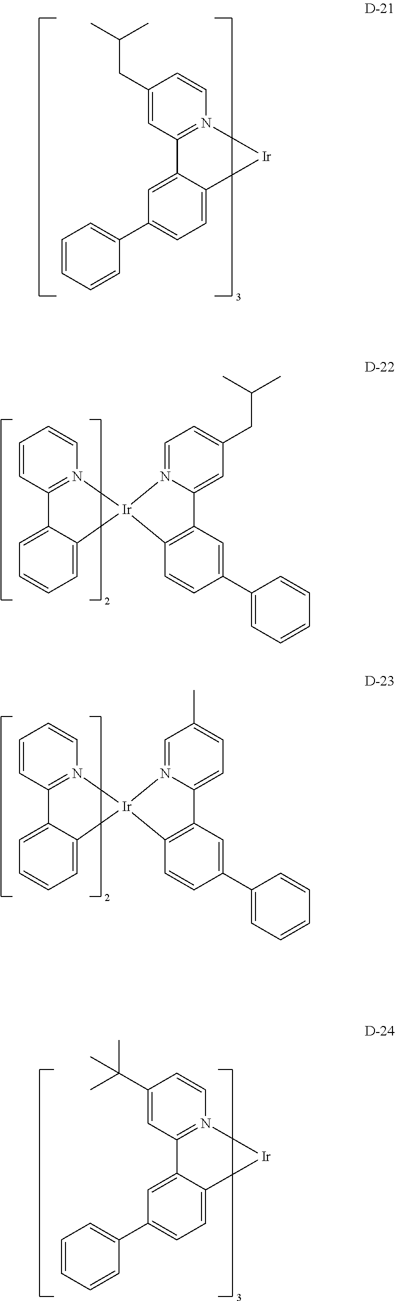

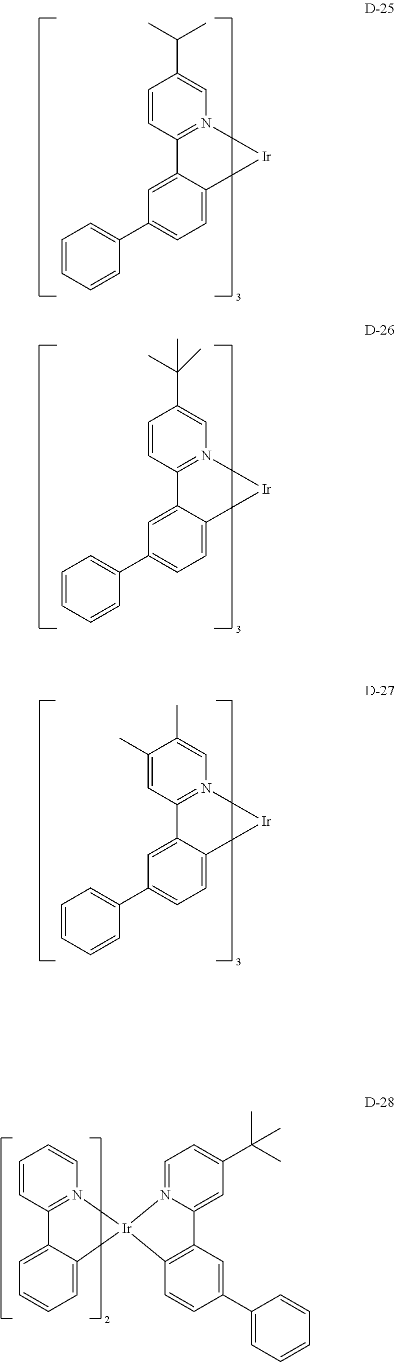

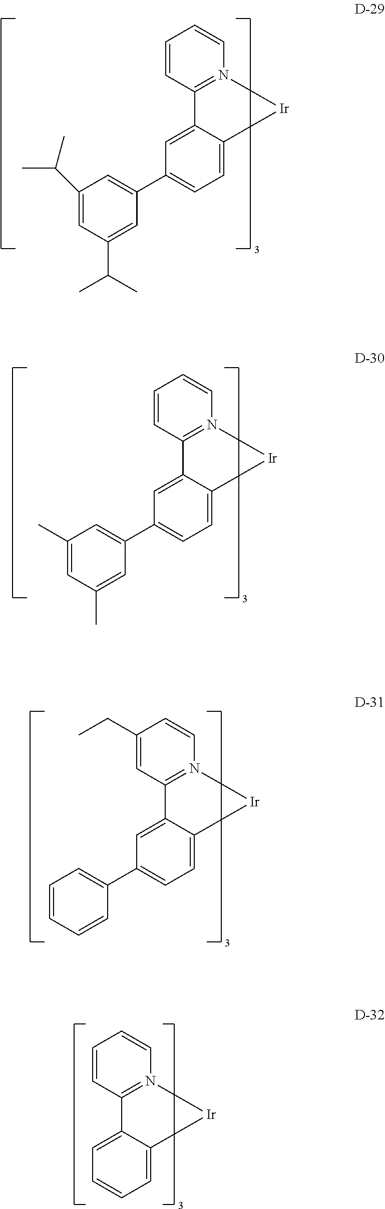

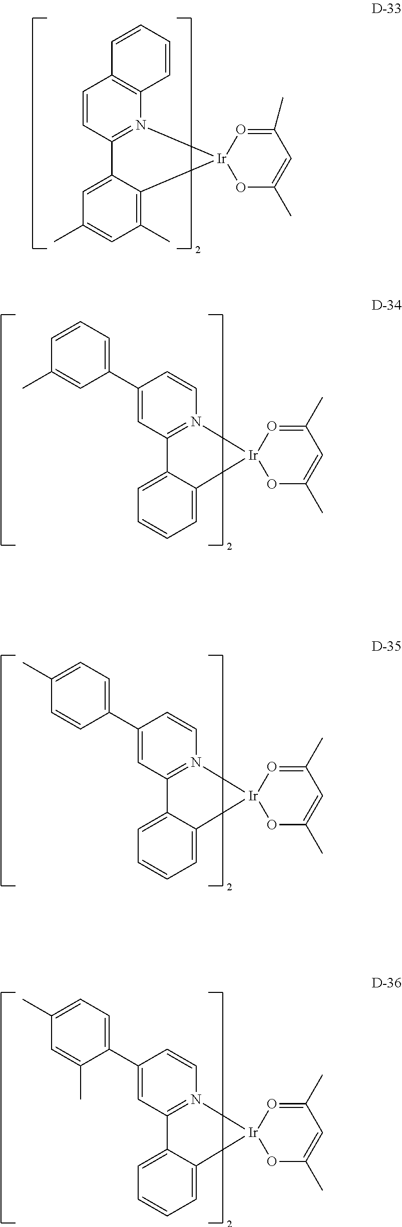

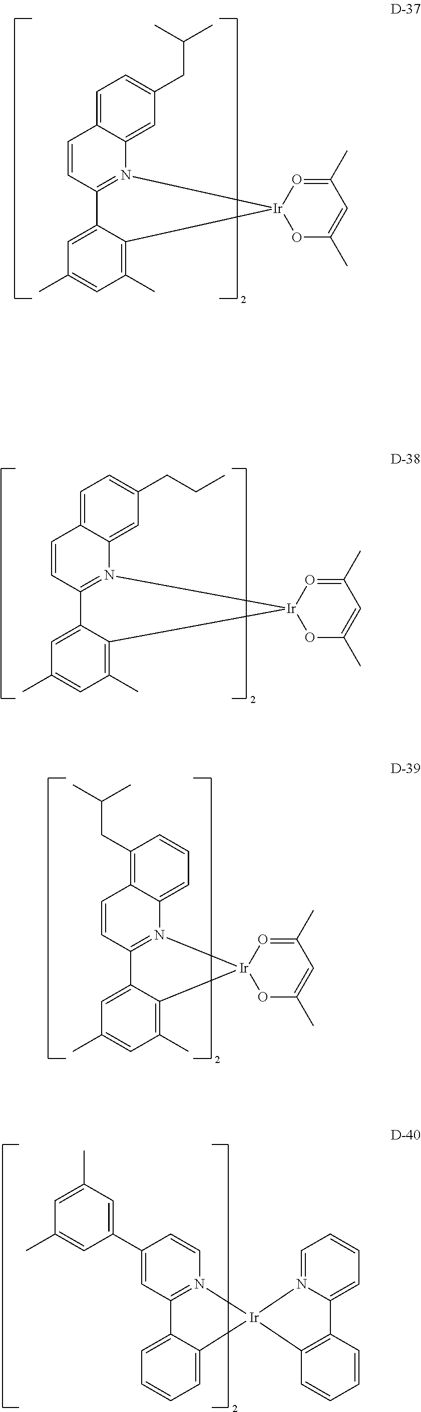

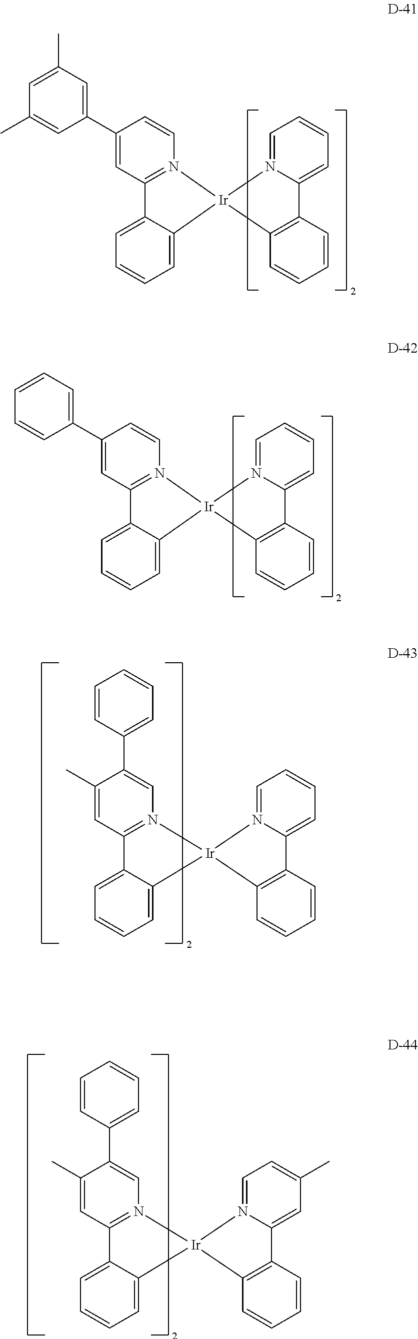

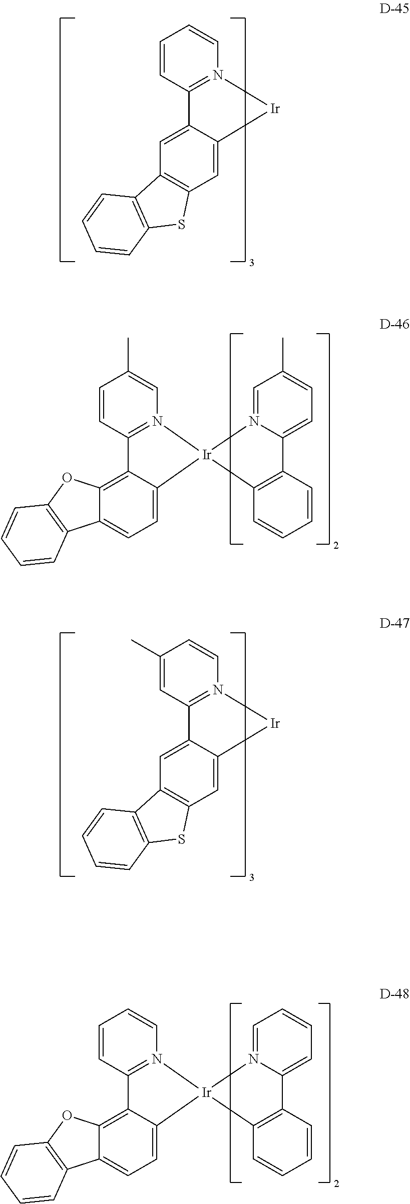

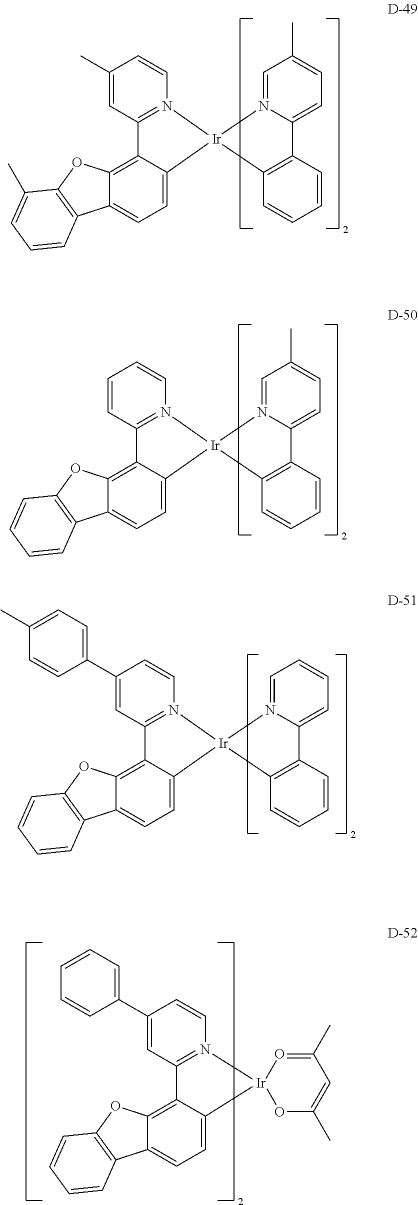

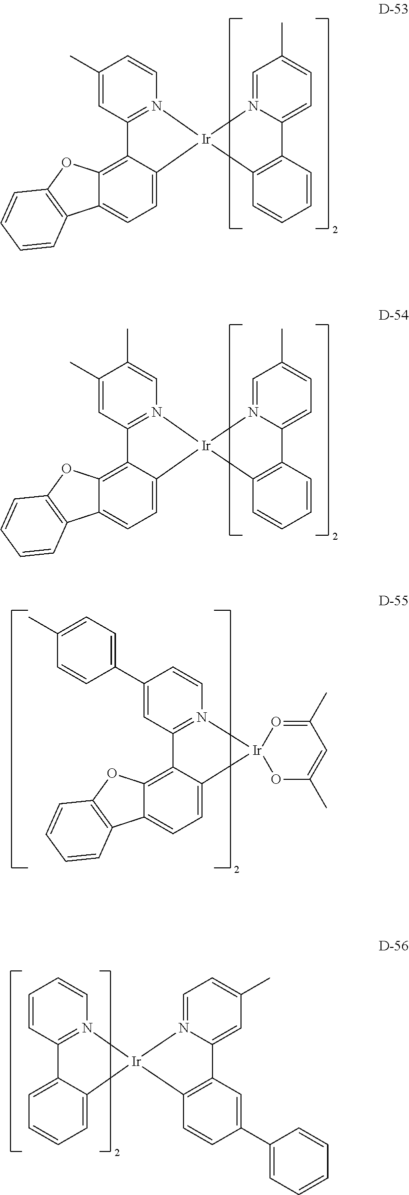

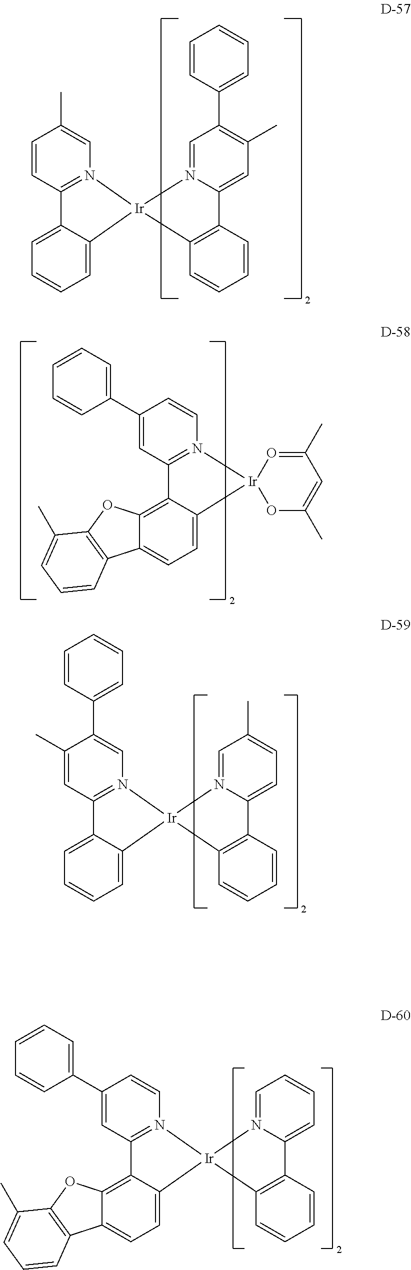

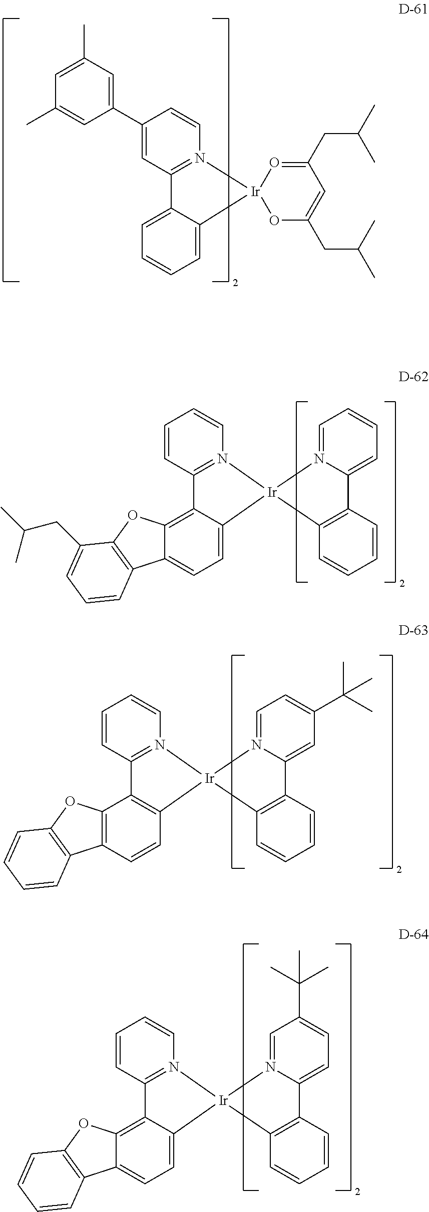

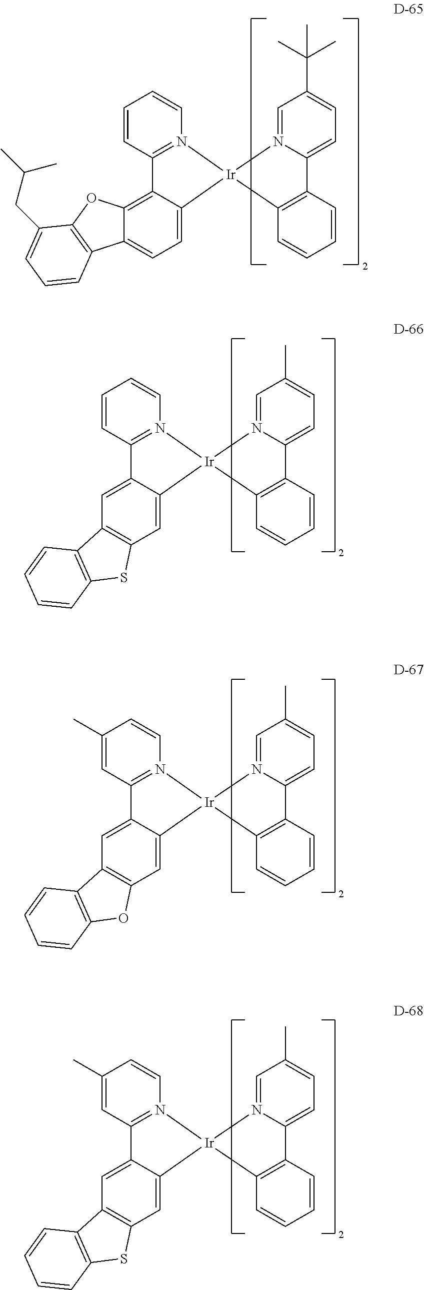

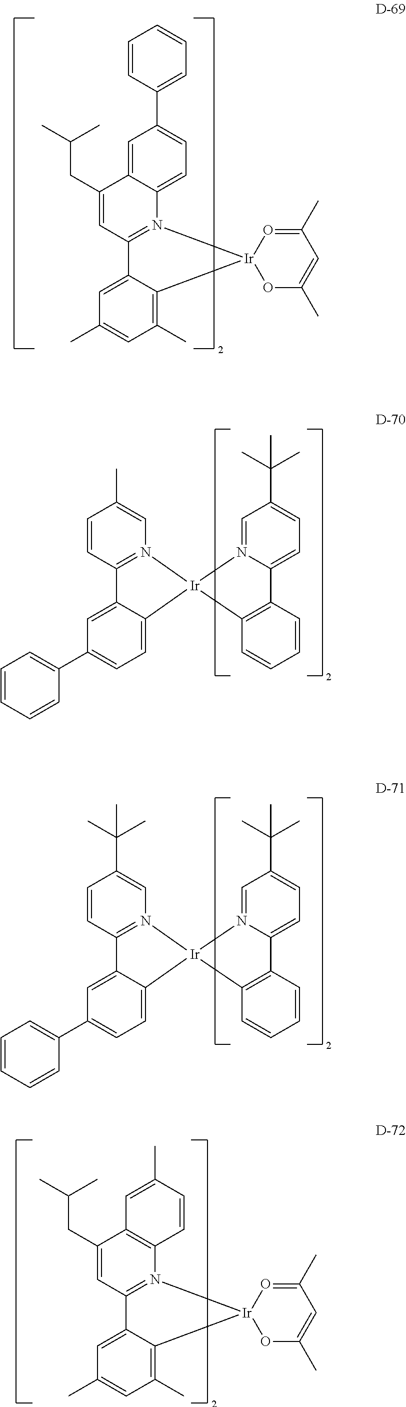

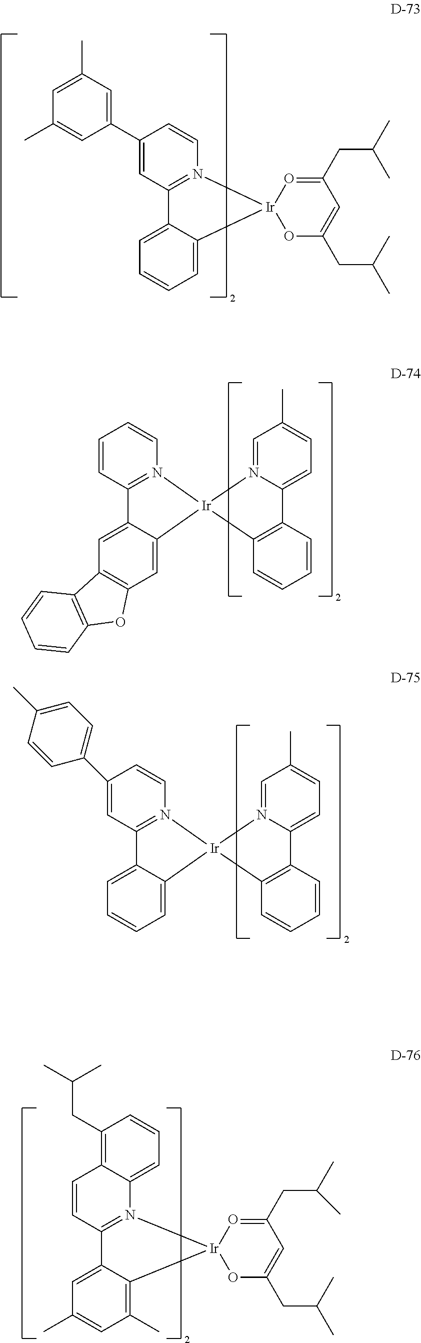

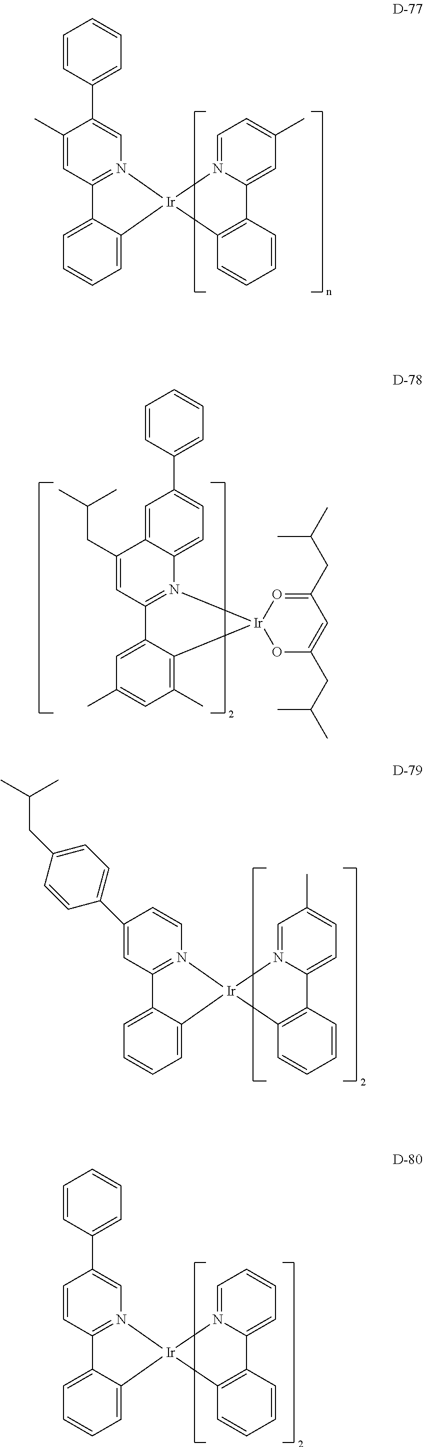

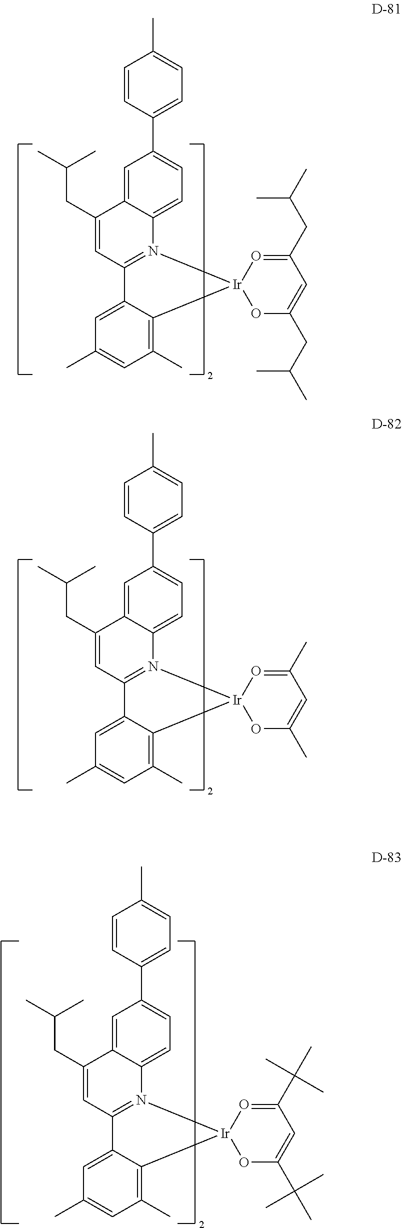

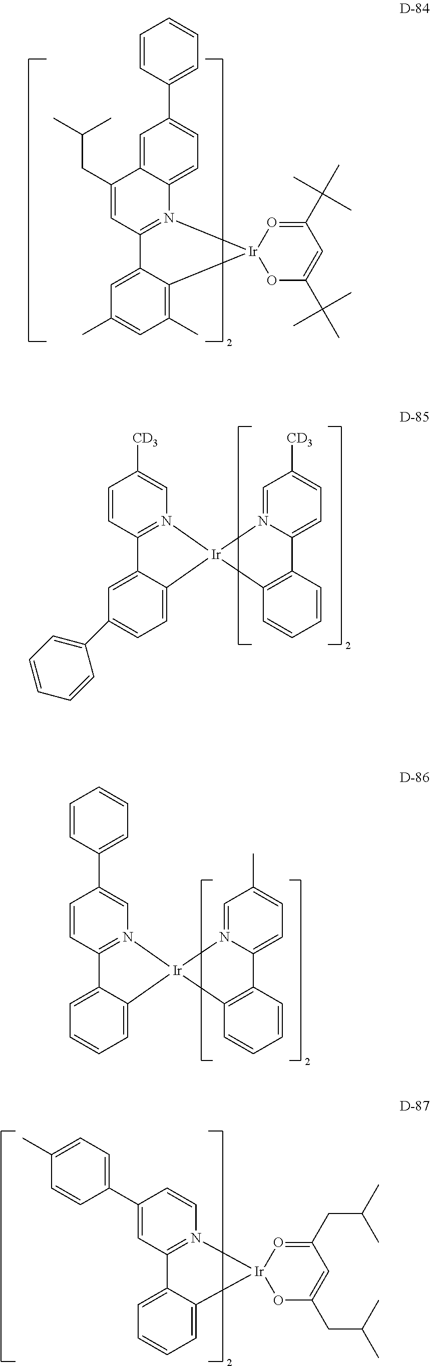

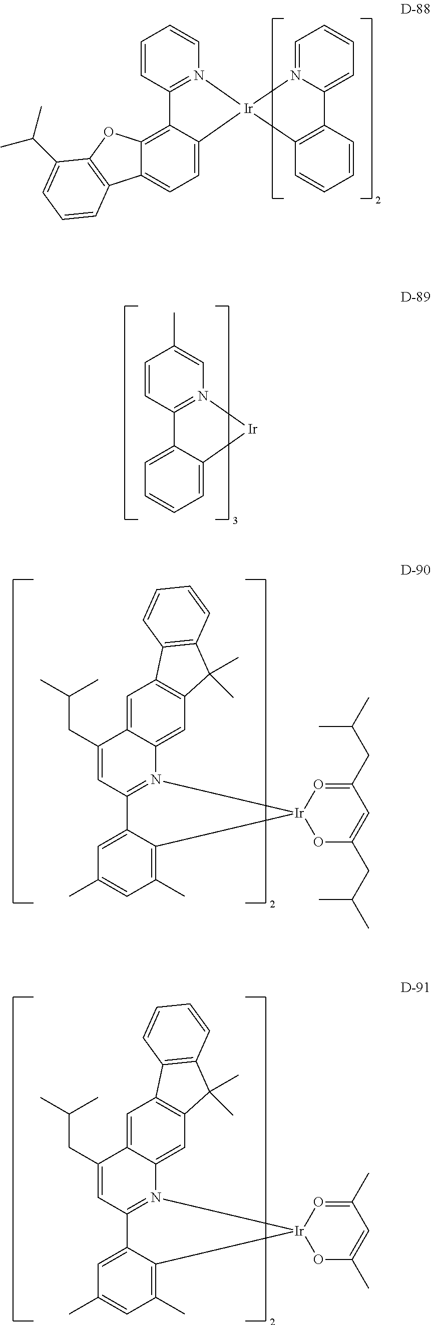

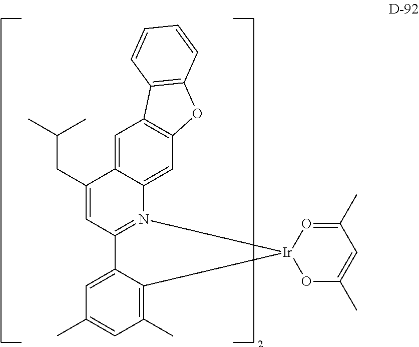

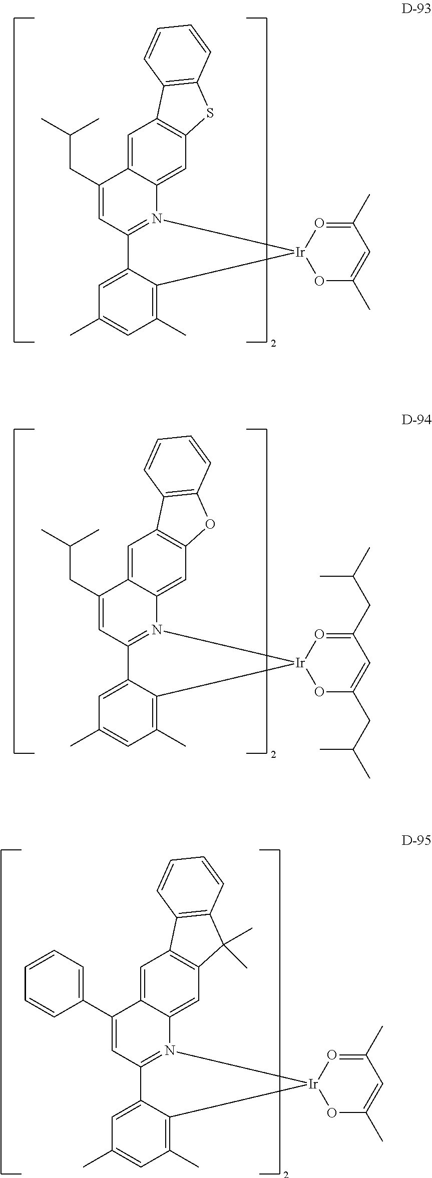

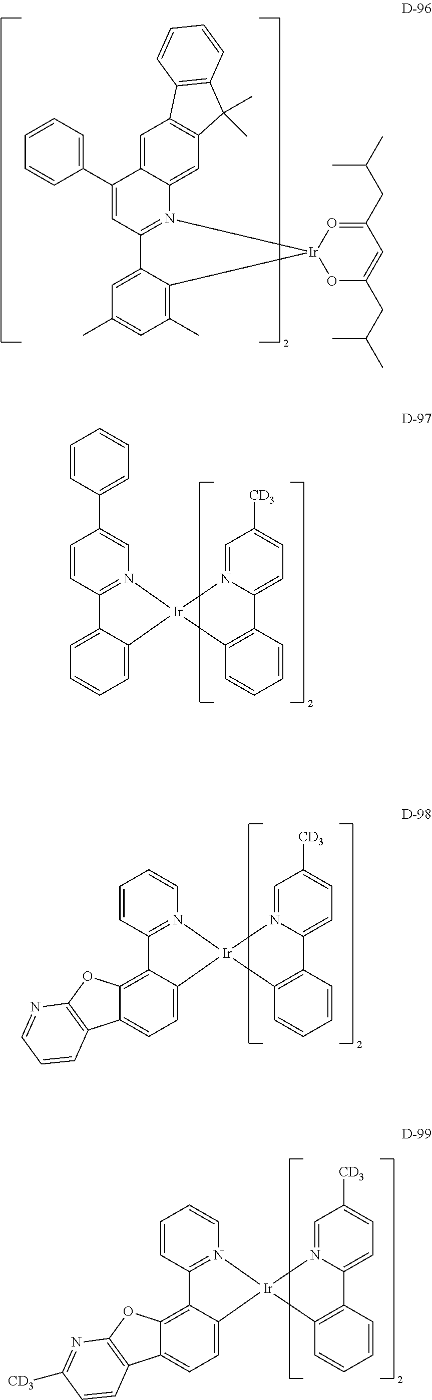

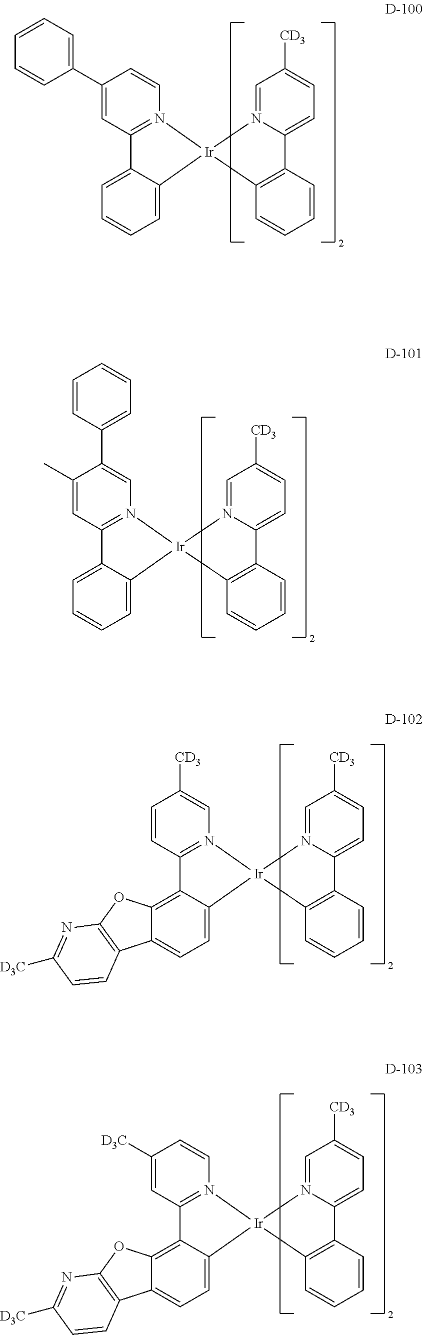

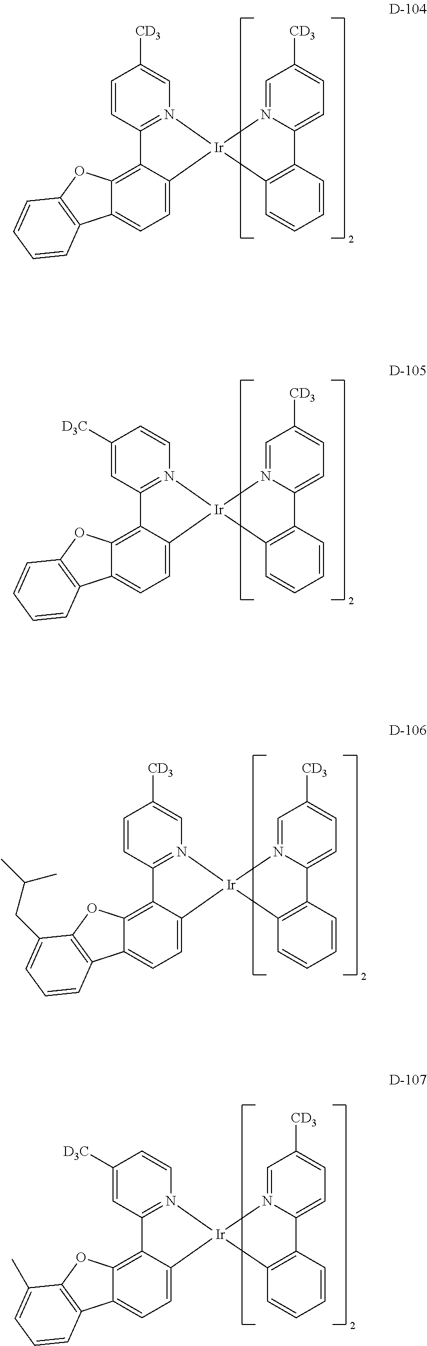

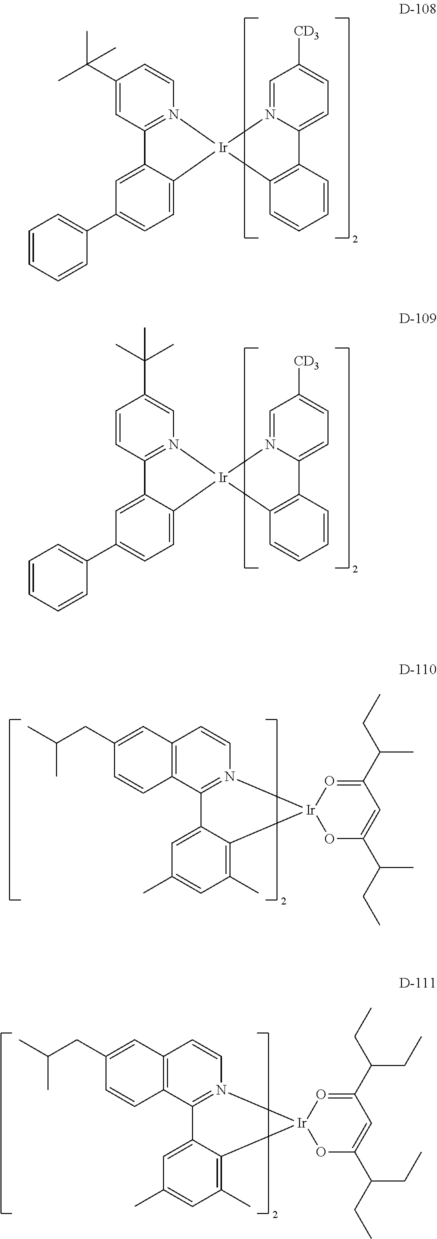

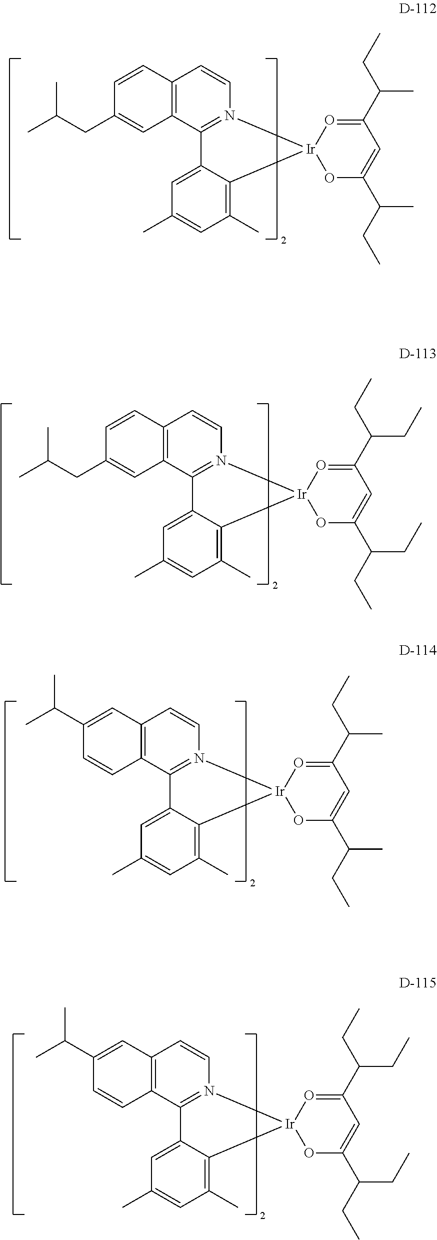

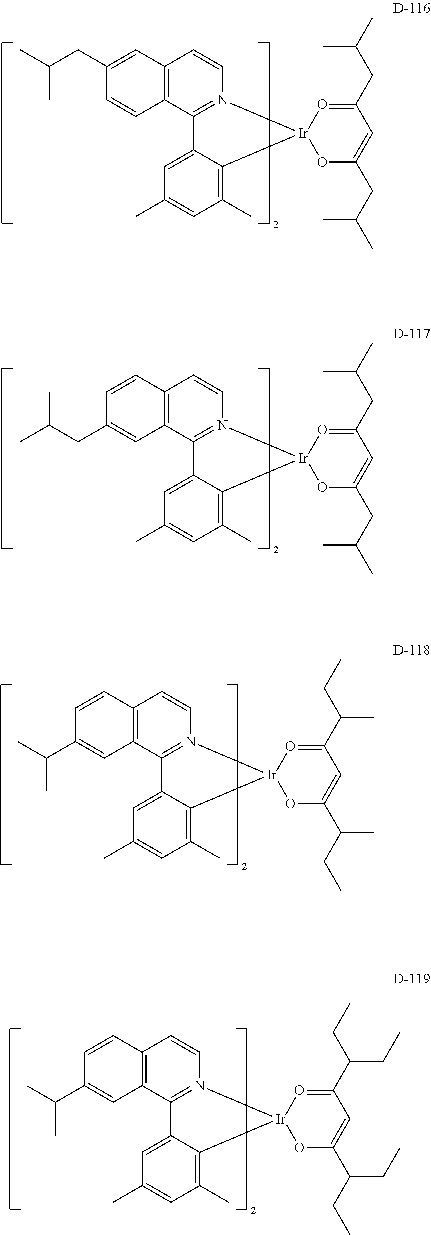

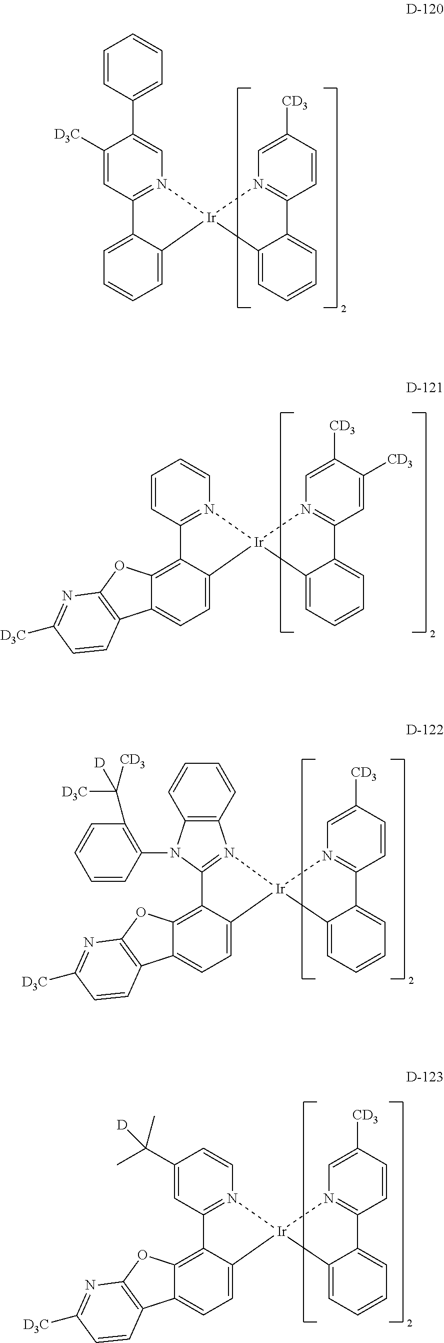

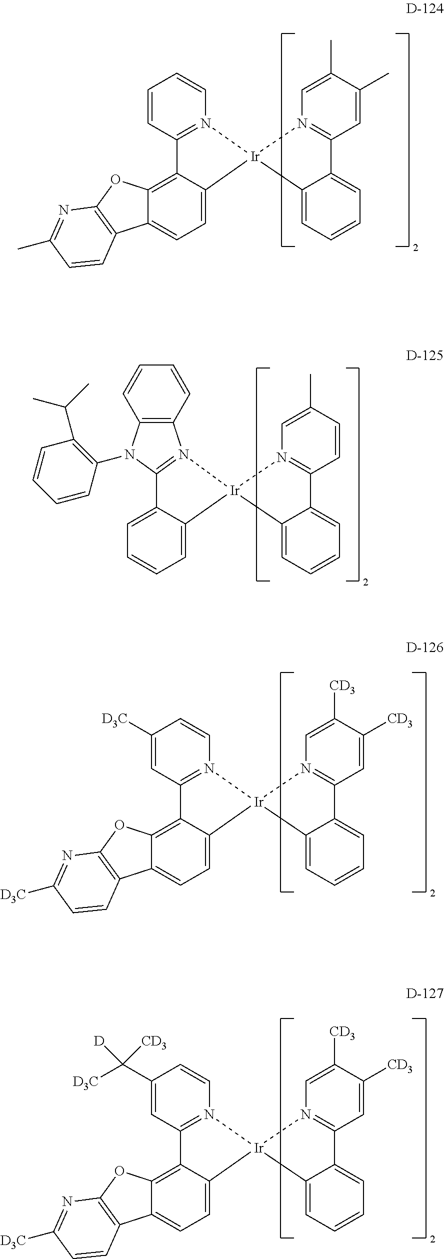

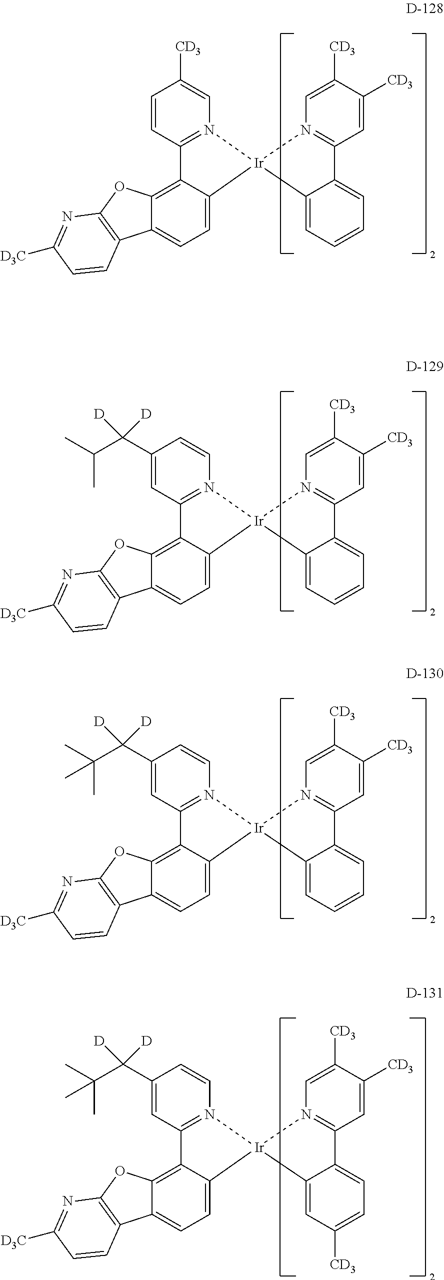

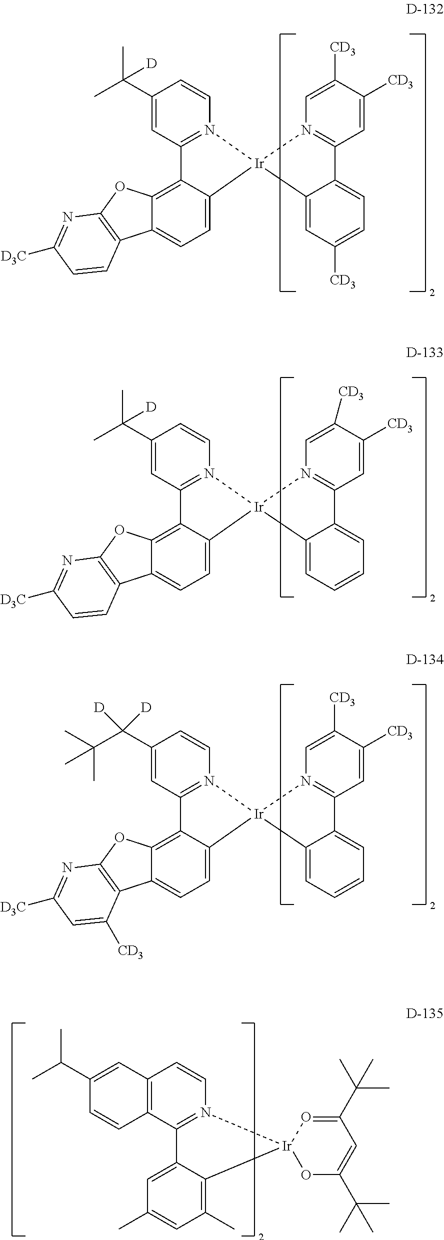

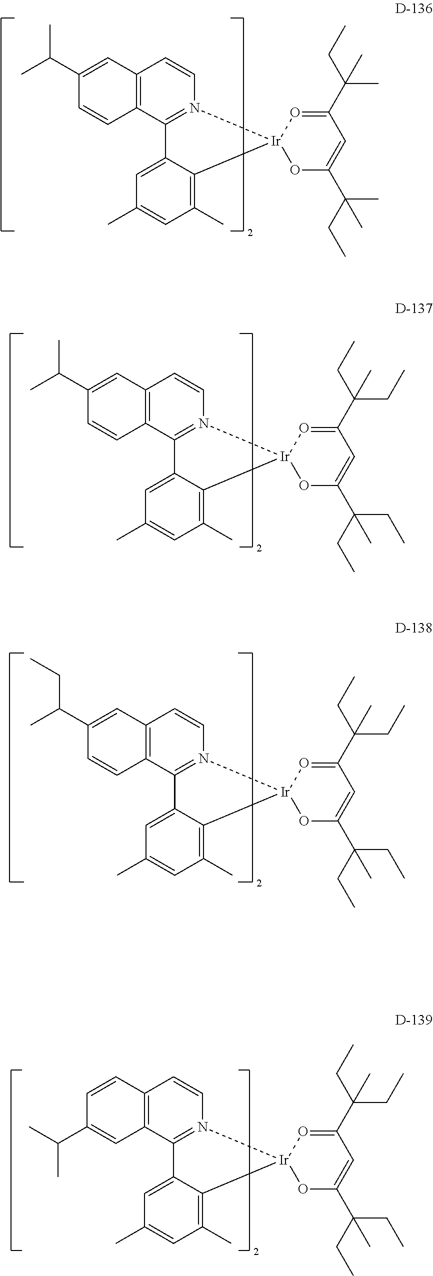

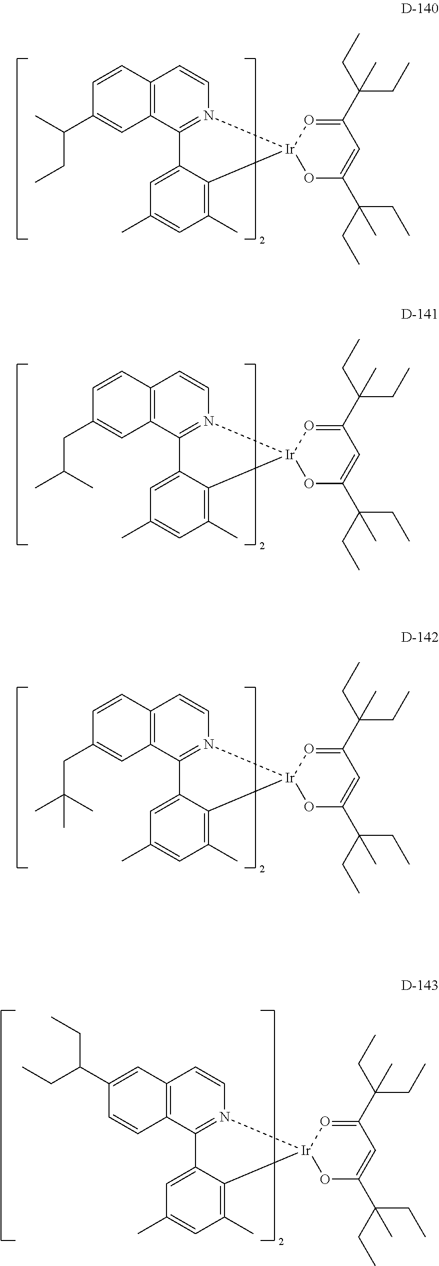

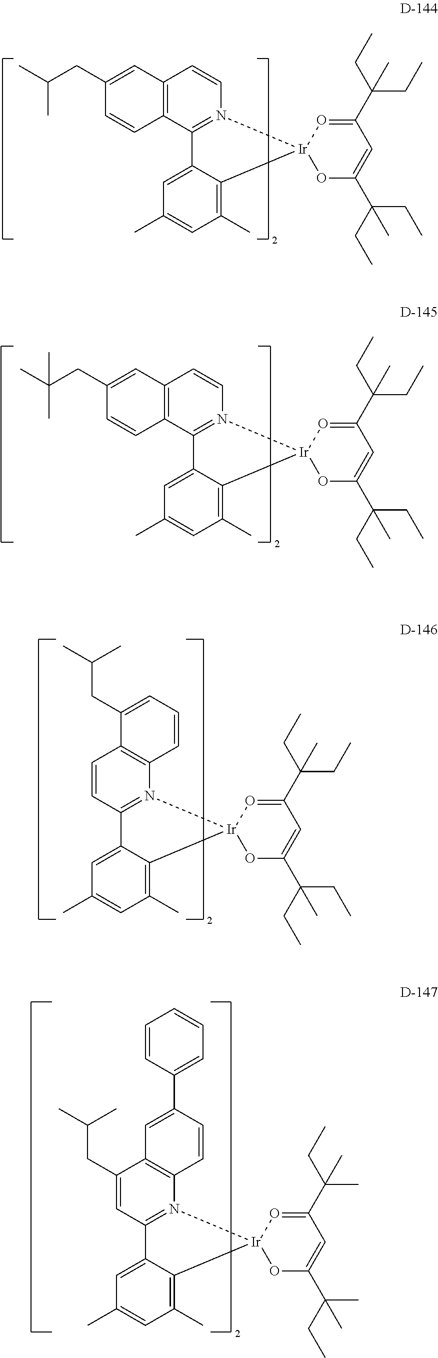

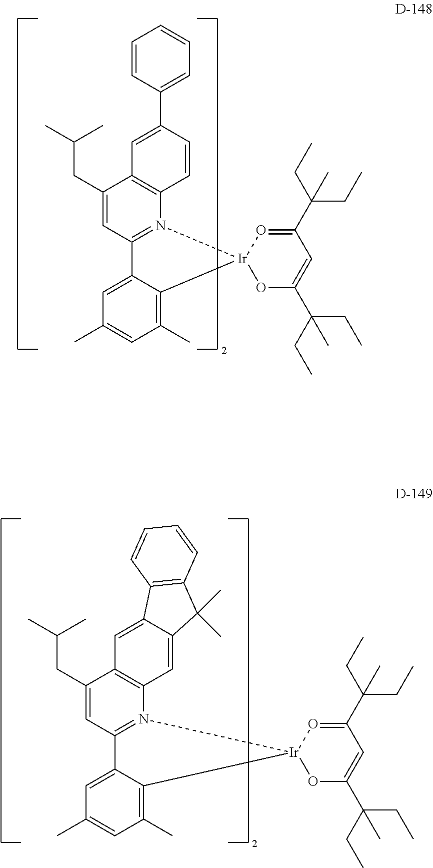

The organic electroluminescent device of the present disclosure may comprise the compound represented by Formula 1, and may further comprise conventional materials included in the organic electroluminescent material. The organic electroluminescent device comprising the compound represented by Formula 1 or a plurality of host materials of the present disclosure may exhibit low driving voltage, high luminous efficiency, and/or long lifetime properties.

The organic electroluminescent material of the present disclosure, e.g., at least one of a hole injection material, a hole transport material, a hole auxiliary material, a light-emitting auxiliary material, an electron blocking material, a light-emitting material, an electron buffer material, a hole blocking material, an electron transport material, an electron injection material and a light-emitting material (host material) may comprise a compound represented by Formula 1. For example, the organic electroluminescent material may be a light-emitting material, and specifically, a host material. The organic electroluminescent material may consist of the compound represented by Formula 1 alone, or may further comprise conventional materials included in the organic electroluminescent material. When two or more materials are comprised in one layer, they may be mixture-evaporated to form a layer, or may be separately co-evaporated at the same time to form a layer.

In addition, the organic electroluminescent material according to one embodiment of the present disclosure may be used as a light-emitting material for a white organic light-emitting device. The white organic light-emitting device has been suggested to have various structures such as a side-by-side structure or a stacking structure depending on the arrangement of R (red), G (green) or YG (yellow green), and B (blue) light-emitting parts, or color conversion material (CCM) method, etc. The present disclosure may also be applied to such a white organic light-emitting device. The organic electroluminescent material according to one embodiment of the present disclosure may also be used in an organic electroluminescent device comprising a quantum dot (QD).

Furthermore, the present disclosure may provide a display system using the compound represented by Formula 1. In other words, it is possible to produce a display system or a lighting system by using the compound of the present disclosure. Specifically, it is possible to produce a display system, e.g., a display system for smart phones, tablets, notebooks, PCs, TVs, or cars; or a lighting system, e.g., an outdoor or indoor lighting system, by using the compound of the present disclosure.

The manufacturing method of the organic electroluminescent device of the present disclosure is not limited, and the manufacturing method of the Device Example as described below is only an example and is not limited thereto. One skilled in the art can reasonably modify the manufacturing method of the Device Examples as described below by relying on existing technology. For example, there is no particular limitation on the mixing ratio of the first host compound and the second host compound, and thus one skilled in the art can reasonably select this within a certain range by depending on existing technology. For example, based on the total weight of the light-emitting layer material, the total weight of the first host compound and the second host compound accounts for 99.5% to 80.0% of the total weight of the light-emitting layer, the weight ratio of the first host compound and the second host compound may be between 1:99 and 99:1, between 20:80 and 99:1, or between 50:50 and 90:10. In the manufacture of devices, when forming a light-emitting layer by co-depositing two or more host materials and a light-emitting material, the two or more host materials and the light-emitting material may each be placed in different evaporation sources and co-deposited to form a light-emitting layer, or a pre-mixed mixture of two or more host materials may be placed on the same evaporation source and then co-deposited with a light-emitting material placed on another evaporation source to form a light-emitting layer. This premixing method can further save evaporation sources. According to one embodiment, the first host compound, the second host compound, and the light-emitting material of the present disclosure may each be placed in different evaporation sources and co-deposited to form a light-emitting layer, or a pre-mixed mixture of the first host compound and the second host compound may be placed in the same evaporation source and then co-deposited with a light-emitting material placed in another evaporation source to form a light-emitting layer.

Hereinafter, the preparation method of the compounds according to the present disclosure, the properties thereof, and the properties of the OLED comprising the organic electroluminescent compound according to the present disclosure will be explained in detail with reference to the representative compounds of the present disclosure. The following examples only describe the properties of the compound and the OLED comprising the same according to the present disclosure, but the present disclosure is not limited to the following examples.

### Example 1: Preparation of Compound C-34

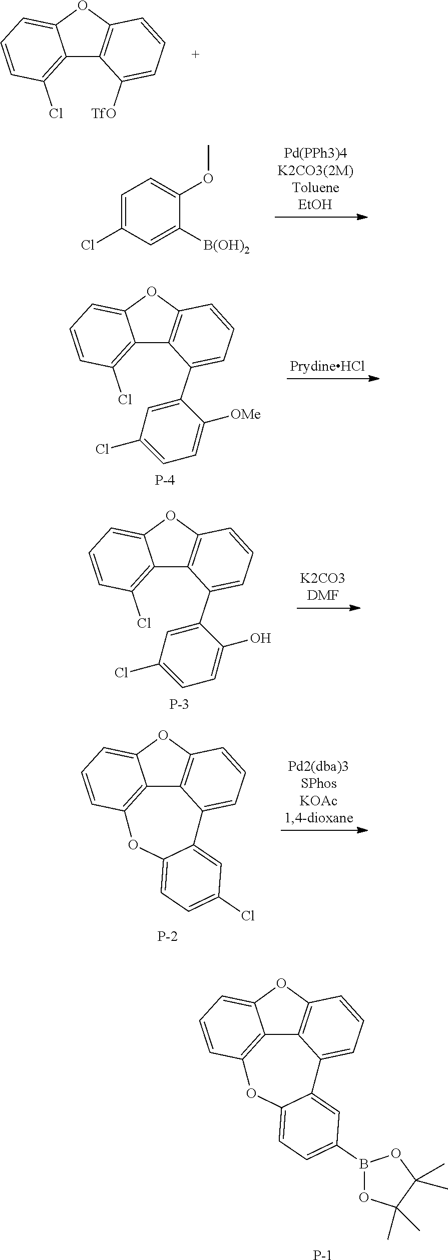

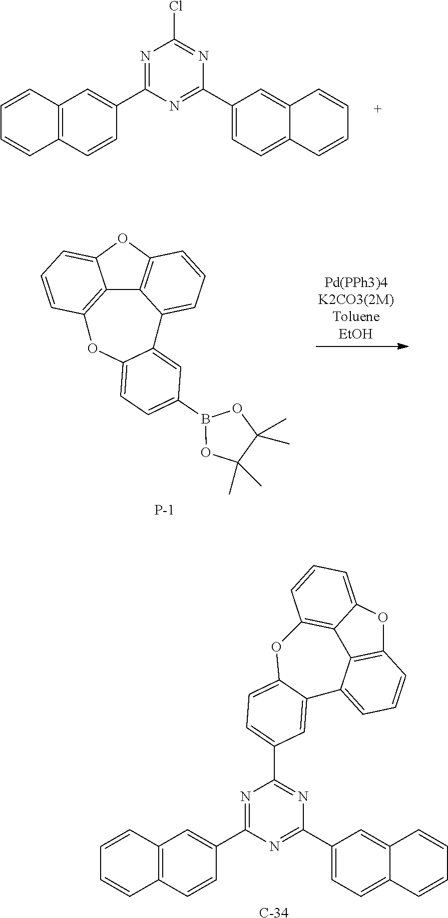

### Synthesis of Compound P-4

In a flask, 9-chlorodibenzo[b,d]furan-1-yl-trifluoromethanesulfonate (20.0 g, 57.15 mmol), (5-chloro-2-methoxyphenyl) boronic acid (11.7 g, 62.86 mmol), tetrakis(triphenylphosphine)palladium(0) (3.3 g, 2.86 mmol), and potassium carbonate (19.75 g, 142.87 mmol) were dissolved in 285 mL of toluene, 71 mL of ethanol, and 71 ml of water, and the mixture was stirred under reflux for 3 hours. After completion of the reaction, the produced solid was filtered and separated by column chromatography to obtain Compound P-4 (19 g, yield: 96.8%).

### Synthesis of Compound P-3

In a flask, Compound P-4 (20 g, 58.28 mmol) and pyridine hydrochloride (18.66 g) were dissolved at 200° C., and the mixture was stirred under reflux for 24 hours. After completion of the reaction, the mixture was cooled to room temperature, and an organic layer was extracted, and then the residual moisture was removed with magnesium sulfate. The residue was separated by column chromatography to obtain Compound P-3 (19 g, yield: 99.05%).

### Synthesis of Compound P-2

In a flask, Compound P-3 (19 g, 57.72 mmol), potassium carbonate (4.79 g, 34.63 mmol), and 384 mL of DMF were added, and the mixture was stirred under reflux at 180° C. for 24 hours. After completion of the reaction, the mixture was cooled to room temperature, and an organic layer was extracted, and then the residual moisture was removed with magnesium sulfate. The residue was separated by column chromatography to obtain Compound P-2 (15 g, yield: 88.78%).

### Synthesis of Compound P-1

In a flask, Compound P-2 (9 g, 30.75 mmol), bis(pinacolato)diboron (10.15 g, 39.97 mmol), tris(dibenzylideneacetone)dipalladium(0) (1.4 g, 1.54 mmol), dicyclohexyl[2′,6′-dimethoxy[1,1′-biphenyl]-2-yl]phosphane (SPhos) (1.26 g, 3.07 mmol), and potassium acetate (7.54 g, 76.87 mmol) were dissolved in 205 mL of 1,4-dioxane, and the mixture was stirred under reflux at 140° C. After completion of the reaction, the mixture was cooled to room temperature, and an organic layer was extracted, and then the residual moisture was removed with magnesium sulfate. The residue was separated by column chromatography to obtain Compound P-1 (10 g, yield: 84.6%).

### Synthesis of Compound C-34

In a flask, Compound P-1 (4.78 g, 12.99 mmol), 2-chloro-4,6-di(naphthalen-2-yl)-1,3,5-triazine (5.49 g, 14.29 mmol), tetrakis(triphenylphosphine)palladium(0) (750 mg, 0.65 mmol), and potassium carbonate (5.39 g, 38.98 mmol) were dissolved in 65 mL of toluene, 16 mL of ethanol, and 16 mL of water, and the mixture was stirred under reflux for 3 hours. After completion of the reaction, the produced solid was filtered and separated by column chromatography to obtain Compound C-34 (5 g, yield: 65.2%).

| | MW | M.P. [° C.] |
|---|---|---|
| C-34 | 589.65 | 299.1 |

### Example 2: Preparation of Compound C-44

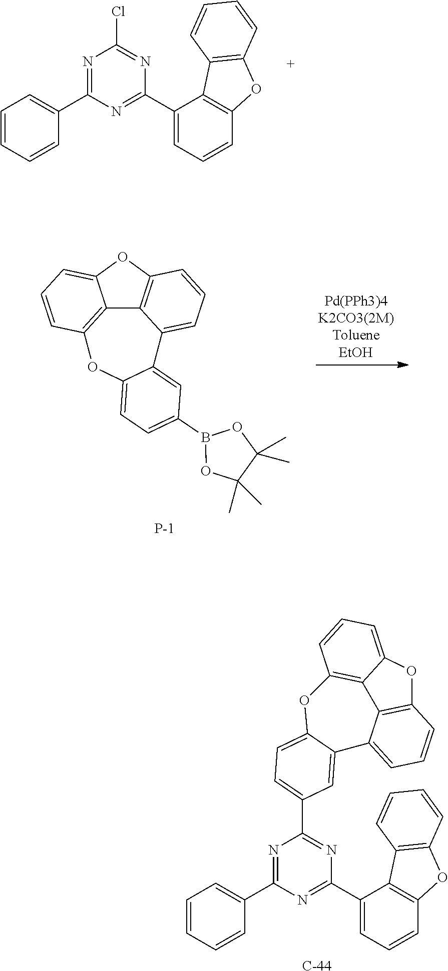

In a flask, Compound P-1 (5.68 g, 15.91 mmol), 2-chloro-4-(dibenzo[b,d]furan-1-yl)-6-phenyl-1,3,5-triazine (6.72 g, 17.5 mmol), tetrakis(triphenylphosphine)palladium(0) (0.92 g, 0.8 mmol), and potassium carbonate (6.6 g, 47.72 mmol) were dissolved in 79 mL of toluene, 19 mL of ethanol, and 19 ml of water, and the mixture was stirred under reflux for 3 hours. After completion of the reaction, the produced solid was filtered and separated by column chromatography to obtain Compound C-44 (10 g, yield: 82%).

| | MW | M.P. [° C.] |
|---|---|---|
| C-44 | 579.62 | 292.6 |

### Example 3: Preparation of Compound C-18

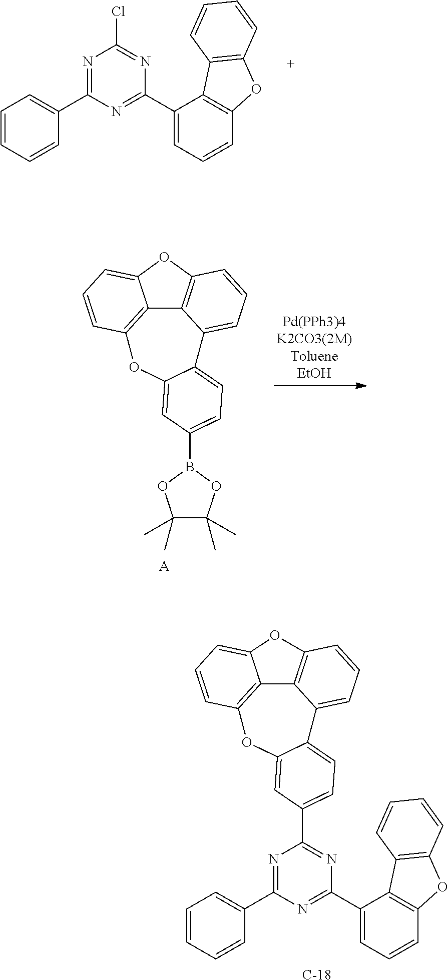

In a flask, Compound A (5.2 g, 14.53 mmol), 2-chloro-4-(dibenzo[b,d]furan-1-yl)-6-phenyl-1,3,5-triazine (6.14 g, 15.99 mmol), tetrakis(triphenylphosphine)palladium(0) (0.84 g, 0.73 mmol), and potassium carbonate (6.03 g, 43.60 mmol) were dissolved in 72 mL of toluene, 18 mL of ethanol, and 18 ml of water, and the mixture was stirred under reflux for 3 hours. After completion of the reaction, the produced solid was filtered and separated by column chromatography to obtain Compound C-18 (4.5 g, yield: 53.4%).

| | MW | M.P. [° C.] |
|---|---|---|
| C-18 | 579.62 | 302.7 |

### Example 4: Preparation of Compound C-148

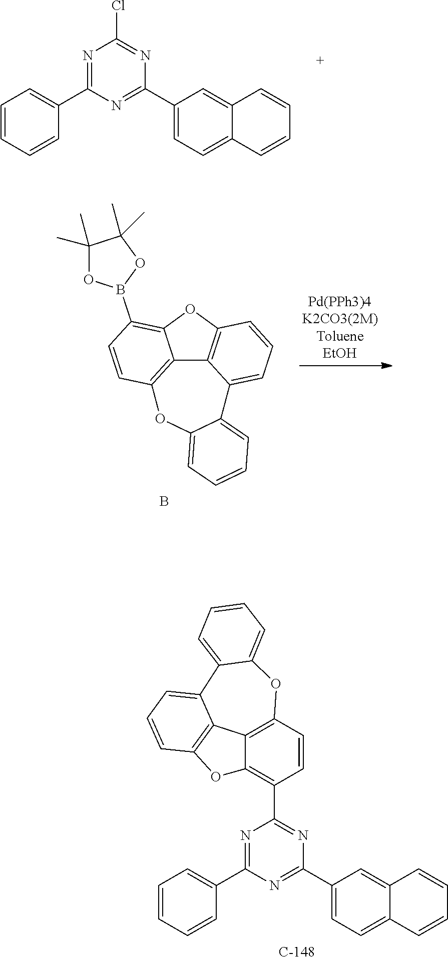

In a flask, Compound B (3.4 g, 10.7 mmol), 2-chloro-4-(naphthalen-2-yl)-6-phenyl-1,3,5-triazine (4.52 g, 11.7 mmol), tetrakis(triphenylphosphine)palladium(0) (0.62 g, 0.53 mmol), and potassium carbonate (4.44 g, 32.10 mmol) were dissolved in 53 mL of toluene, 13 mL of ethanol, and 13 ml of water, and the mixture was stirred under reflux for 3 hours. After completion of the reaction, the produced solid was filtered and separated by column chromatography to obtain compound C-148 (1.3 g, yield: 22.5%).

| | MW | M.P. [° C.] |
|---|---|---|
| C-148 | 539.59 | 265.1 |

### Example 5: Preparation of Compound C-33

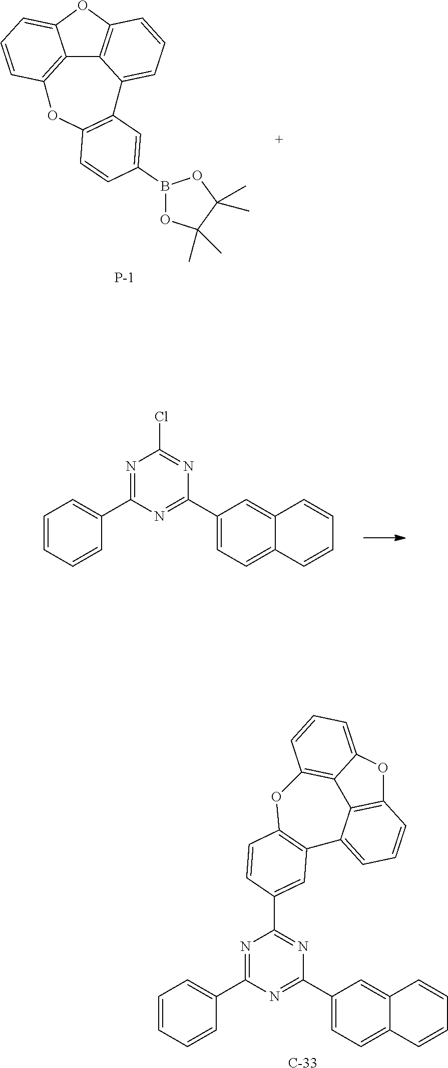

In a flask, Compound P-1 (7.4 g, 23.29 mmol), 2-chloro-4-(naphthalen-2-yl)-6-phenyl-1,3,5-triazine (9.84 g, 25.62 mmol), tetrakis(triphenylphosphine)palladium(0) (0.81 g, 0.7 mmol), and potassium carbonate (8.05 g, 58.22 mmol) were dissolved in 116 mL of toluene, 29 mL of ethanol, and 29 ml of water, and the mixture was stirred under reflux for 3 hours. After completion of the reaction, the produced solid was filtered and separated by column chromatography to obtain Compound C-33 (4 g, yield: 31.8%).

| | MW | M.P. [° C.] |
|---|---|---|
| C-33 | 539.59 | 274.6 |

### Example 6: Preparation of Compound C-190

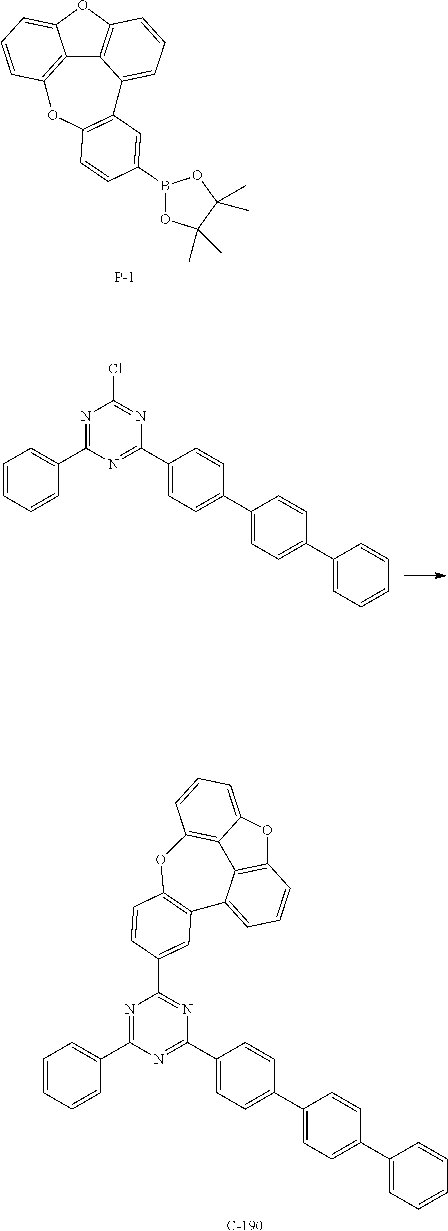

In a flask, Compound P-1 (7.4 g, 17.62 mmol), 2-([1,1′:4′,1″-terphenyl]-4-yl)-4-chloro-6-phenyl-1,3,5-triazine (8.8 g, 1.3 mmol), tetrakis(triphenylphosphine)palladium(0) (0.61 g, 0.53 mmol), and potassium carbonate (6.1 g, 44.06 mmol) were dissolved in 88 mL of toluene, 22 mL of ethanol, and 22 ml of water, and the mixture was stirred under reflux for 3 hours. After completion of the reaction, the produced solid was filtered and separated by column chromatography to obtain Compound C-190 (5 g, yield: 44.2%).

| | MW | M.P. [° C.] |
|---|---|---|
| C-190 | 641.73 | 313.3 |

### Example 7: Preparation of Compound C-172

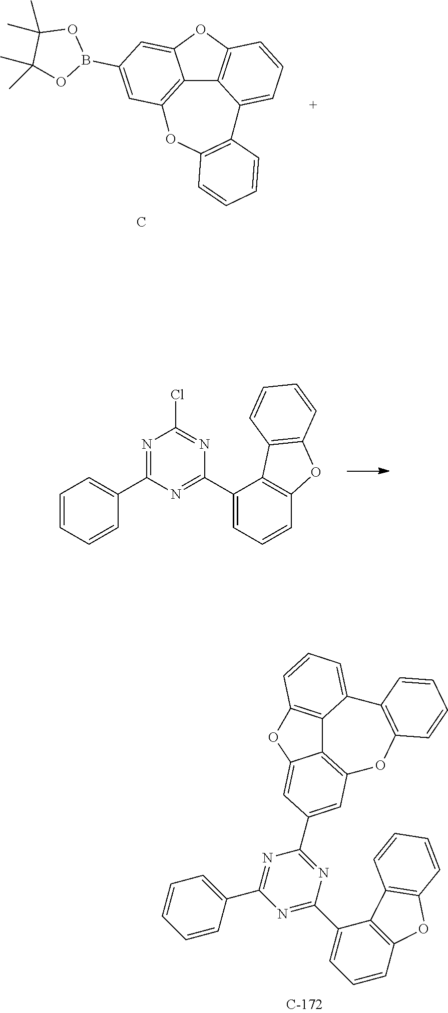

In a flask, Compound C (8.65 g, 20.60 mmol), 2-chloro-4-(dibenzo[b,d]furan-1-yl)-6-phenyl-1,3,5-triazine (6.7 g, 18.73 mmol), tetrakis(triphenylphosphine)palladium(0) (1.08 g, 0.94 mmol), and potassium carbonate (7.76 g, 56.18 mmol) were dissolved in 93 mL of toluene, 23 mL of ethanol, and 23 ml of water, and mixture was stirred under reflux for 3 hours. After completion of the reaction, the produced solid was filtered and separated by column chromatography to obtain Compound C-172 (8 g, yield: 73.7%).

| | MW | M.P. [° C.] |
|---|---|---|
| C-172 | 579.62 | 265.1 |

### Example 8: Preparation of Compound H1-34

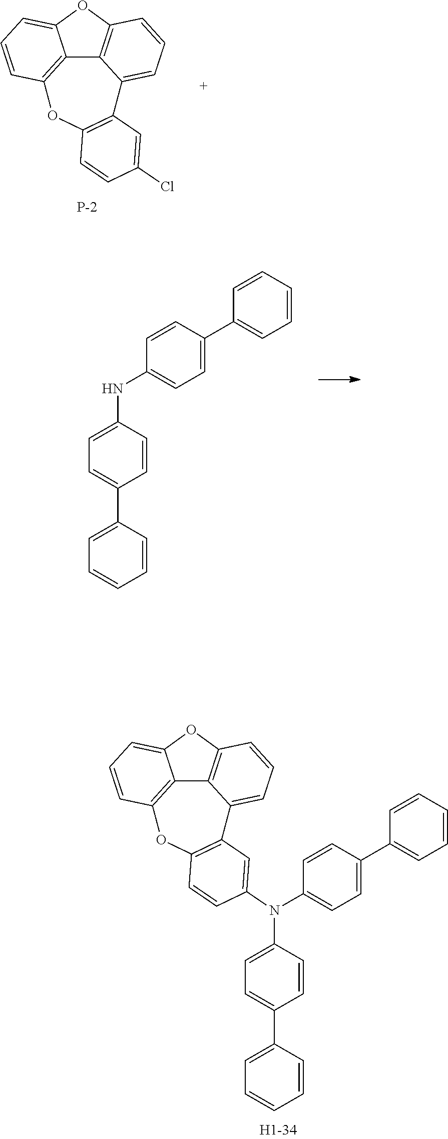

In a flask, Compound P-2 (2.6 g, 8.88 mmol), di([1,1′-biphenyl]-4-yl)amine (3.43 g, 10.66 mmol), tris(dibenzylideneacetone)dipalladium(0) (810 mg, 0.89 mmol), dicyclohexyl[2′,6′-dimethoxy[1,1′-biphenyl]-2-yl]phosphane (SPhos) (730 mg, 1.78 mmol), and sodium tert-butoxide (1.71 g, 17.76 mmol) were dissolved in 45 mL of xylene, and the mixture was stirred under reflux at 180° C. for 1 hour. After completion of the reaction, an organic layer was extracted, and the residual moisture was removed with magnesium sulfate. The residue was separated by column chromatography to obtain Compound H1-34 (1.0 g, yield: 19.49%).

| | MW | M.P. [° C.] |
|---|---|---|
| H1-34 | 577.68 | 109.9 |

### Example 9: Preparation of Compound H1-31

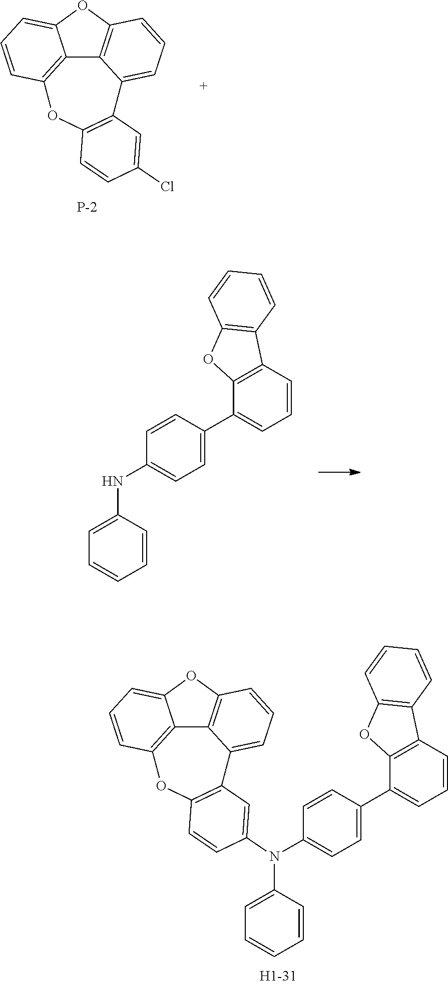

In a flask, Compound P-2 (6 g, 20.50 mmol), 4-(dibenzo[b,d]furan-4-yl)-N-phenyl aniline (8.25 g, 24.6 mmol), tris(dibenzylideneacetone)dipalladium(0) (1.88 g, 2.05 mmol), dicyclohexyl[2′,6′-dimethoxy[1,1′-biphenyl]-2-yl]phosphane (SPhos) (1.68 g, 4.10 mmol), and sodium tert-butoxide (3.94 g, 40.99 mmol) were dissolved in 102 mL of xylene, and the mixture was stirred under reflux at 180° C. for 1 hour. After completion of the reaction, an organic layer was extracted, and the residual moisture was removed with magnesium sulfate. The residue was separated by column chromatography to obtain Compound H1-31 (6.0 g, yield: 49.47%).

| | MW | M.P. [° C.] |
|---|---|---|
| H1-31 | 591.67 | 172.6 |

### Example 10: Preparation of Compound H1-45

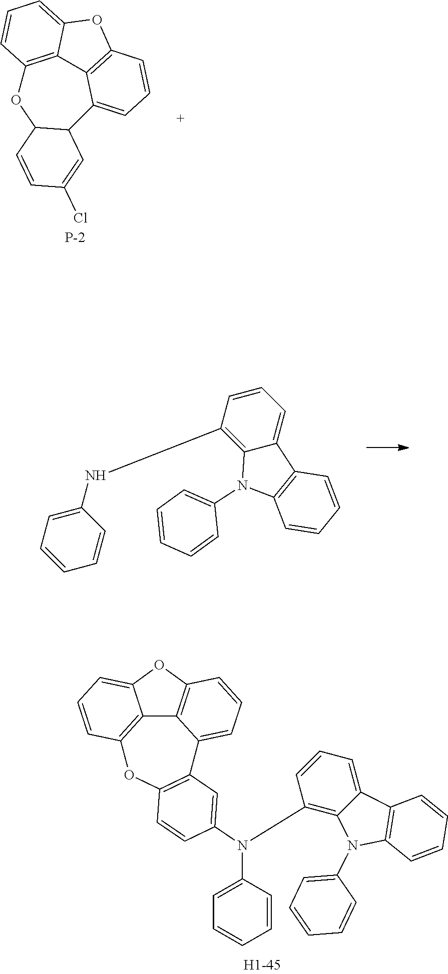

In a flask, Compound P-2 (6 g, 20.497 mmol), N,9-diphenyl-9H-carbazole-1-amine (7.5 g, 22.547 mmol), sodium tert-butoxide (4.9 g, 51.242 mmol), dicyclohexyl[2′,6′-dimethoxy[1,1′-biphenyl]-2-yl]phosphane (SPhos) (0.84 g, 2.049 mmol), and tris(dibenzylideneacetone)dipalladium(0) (0.94 g, 1.025 mmol) were dissolved in 100 mL of xylene, and the mixture was stirred under reflux at 160° C. for 12 hours. After completion of the reaction, an organic layer was extracted with ethyl acetate, and the residual moisture was removed with magnesium sulfate. The residue was dried and separated by column chromatography to obtain Compound H1-45 (1.9 g, yield: 16%).

| | MW | M.P. [° C.] |
|---|---|---|
| H1-45 | 539.59 | 274.6 |

### Example 11: Preparation of Compound H1-73

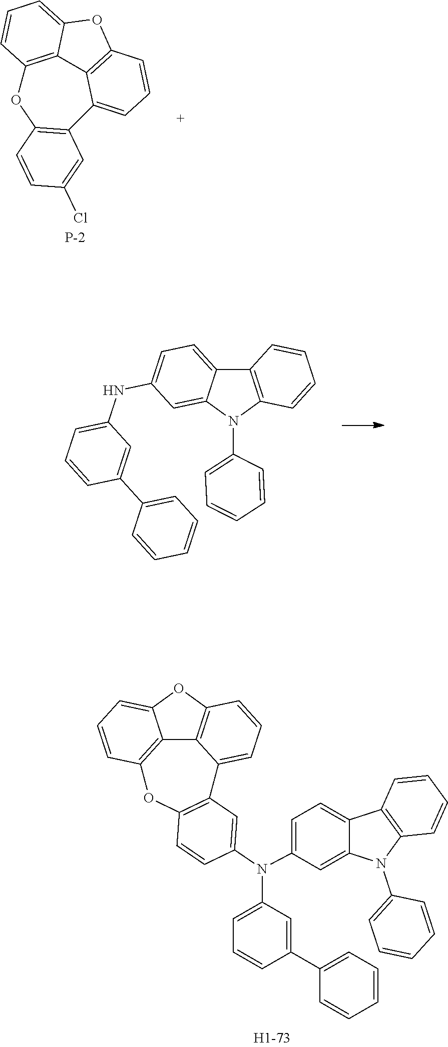

In a flask, Compound P-2 (6 g, 20.497 mmol), N-([1,1′-biphenyl]-3-yl)-9-phenyl-9H-carbazole-2-amine (8.4 g, 20.497 mmol), sodium tert-butoxide (4.9 g, 51.242 mmol), dicyclohexyl[2′,6′-dimethoxy[1,1′-biphenyl]-2-yl]phosphane (SPhos) (0.84 g, 2.049 mmol), and tris(dibenzylideneacetone)dipalladium(0) (0.94 g, 1.025 mmol) were dissolved in 100 ml of xylene, and the mixture was stirred under reflux at 160° C. for 12 hours. After completion of the reaction, an organic layer was extracted with ethyl acetate, and the residual moisture was removed with magnesium sulfate. The residue was dried and separated by column chromatography to obtain Compound H1-73 (4.0 g, yield: 31%).

| | MW | M.P. [° C.] |
|---|---|---|
| H1-73 | 666.78 | 216.0 |

### Device Example 1: Producing an OLED Comprising the Compound According to the Present Disclosure as a Host

An OLED according to the present disclosure was produced. A transparent electrode indium tin oxide (ITO) thin film (10 Ω/sq) on a glass substrate for an OLED (GEOMATEC CO., LTD.) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 in Table 2 was introduced into a cell of the vacuum vapor deposition apparatus, and Compound HT-1 was introduced into another cell of the vacuum vapor deposition apparatus. The two materials were evaporated at different rates, and Compound HI-1 was deposited in a doping amount of 3 wt % based on the total amount of Compound HI-1 and Compound HT-1 to form a hole injection layer having a thickness of 10 nm. Next, Compound HT-1 was deposited on the hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: The host compound shown in Table 1 below was introduced into two cells of the vacuum vapor deposition apparatus as a host, and Compound D-39 was introduced into another cell as a dopant. The host material was evaporated and the dopant material was simultaneously evaporated at a different rate, and the dopant was deposited in a doping amount of 3 wt % based on the total amount of the host and the dopant to form a light-emitting layer having a thickness of 40 nm on the second hole transport layer. Thereafter, Compound ET-1 and Compound EI-1 were evaporated in a weight ratio of 50:50 to deposit an electron transport layer having a thickness of 35 nm on the light-emitting layer. After depositing Compound EI-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10-6 Torr.

### Comparative Examples 1 and 2: Producing an OLED Comprising a Conventional Compound as a Host

An OLED was produced in the same manner as in Device Example 1, except that the host compound shown in Table 1 below was used as a host of the light-emitting layer.

The driving voltage, luminous efficiency, and light-emitting color at a luminance of 1,000 nit, and the time taken for luminance to decrease from 100% to 95% (lifetime; T95) at a luminance of 10,000 nit of the OLEDs produced in Device Example 1, and Comparative Examples 1 and 2 are provided in Table 1 below.

TABLE 1

| | | Driving | Luminous | Light- | |
|---|---|---|---|---|---|
| | | Voltage | Efficiency | Emitting | Lifetime |
| | Host | [V] | [cd/A] | Color | T95 [hr] |
| Comparative | X-1 | 3.0 | 26.3 | Red | 14 |
| Example 1 | | | | | |
| Comparative | X-2 | 4.0 | 29.8 | Red | 0.1 |
| Example 2 | | | | | |
| Device | C-44 | 2.9 | 29.2 | Red | 36 |
| Example 1 | | | | | |

From Table 1 above, it can be confirmed that the organic electroluminescent device comprising the compound according to the present disclosure exhibits longer lifetime properties, while exhibiting driving voltages and/or luminous efficiencies equivalent to or higher than those of conventional organic electroluminescent devices.

### Device Example 2: Producing an OLED by Co-Depositing a First Host Compound and a Second Host Compound According to the Present Disclosure

An OLED was produced in the same manner as in Device Example 1, except that the host compounds shown in Table 2 below were evaporated at a rate of 1:1 and used as hosts of the light-emitting layer.

### Comparative Example 3: Producing an OLED Comprising a Comparative Compound as a Host

An OLED was produced in the same manner as in Device Example 2, except that the compound shown in Table 2 below was used as the first host of the light-emitting layer.

The light-emitting color and luminous efficiency at a luminance of 5,000 nit of the OLEDs produced in Device Example 2 and Comparative Example 3 are provided in Table 2 below.

TABLE 2

| | | | Light- | Luminous |
|---|---|---|---|---|
| | | Second | Emitting | Efficiency |
| | First Host | Host | Color | [cd/A] |
| Device Example 2 | H1-31 | H2-26 | Red | 33.2 |
| Comparative | X-3 | H2-26 | Red | 31.7 |
| Example 3 | | | | |

From Table 2 above, it can be confirmed that the organic electroluminescent device comprising a plurality of host materials according to the present disclosure exhibits improved luminous efficiency properties compared to the conventional organic electroluminescent device.

### Device Example 3: Producing an OLED by Co-Depositing a First Host Compound and a Second Host Compound According to the Present Disclosure

An OLED was produced in the same manner as in Device Example 2, except that the compounds shown in Table 3 below were used as hosts of the light-emitting layer.

The light-emitting color and luminous efficiency at a luminance of 5,000 nit of the OLED produced in Device Example 3 are provided in Table 3 below.

TABLE 3

| | | | Light- | Luminous |
|---|---|---|---|---|
| | | | Emitting | Efficiency |
| | First Host | Second Host | Color | [cd/A] |
| Device Example 3 | C-212 | H3-144 | Red | 32.0 |

From Table 3 above, it can be confirmed that the organic electroluminescent device comprising a plurality of host materials according to the present disclosure exhibits high luminous efficiency properties.

The compounds used in the Device Examples and the Comparative Examples are shown in Table 4 below.

TABLE 4

| | |
|---|---|
| Hole Injection Layer/ Hole Transport Layer | 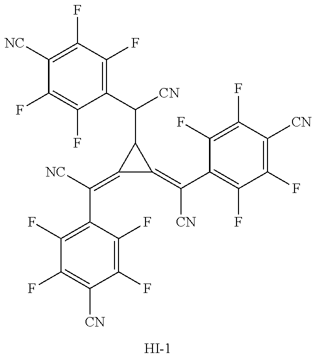 [SMILES: N#C/C(=C1\C(=C(\C#N)c2c(F)c(F)c(C#N)c(F)c2F)C1C(C#N)c1c(F)c(F)c(C#N)c(F)c1F)c1c(F)c(F)c(C#N)c(F)c1F ; [I-]] |
| | 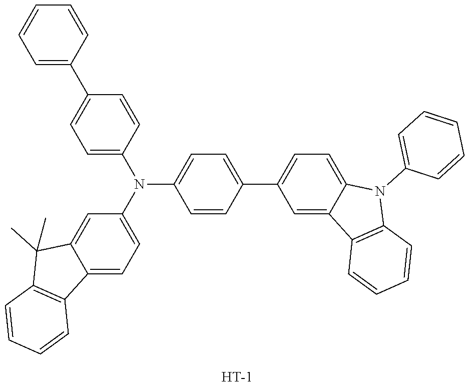 |
| | 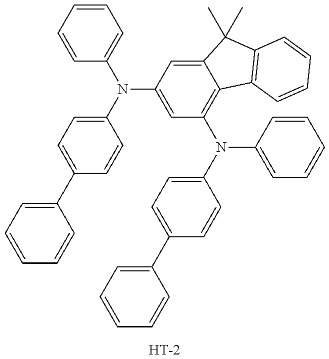 |
| Light- Emitting Layer | 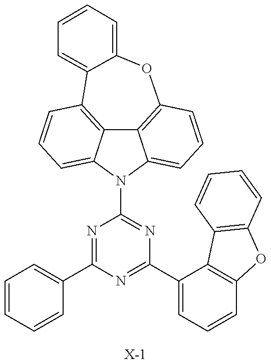 [SMILES: c1ccc(-c2nc(-c3cccc4oc5ccccc5c34)nc(-n3c4cccc5oc6ccccc6c6cccc3c6c54)n2)cc1 ; "X-1"] |
| | 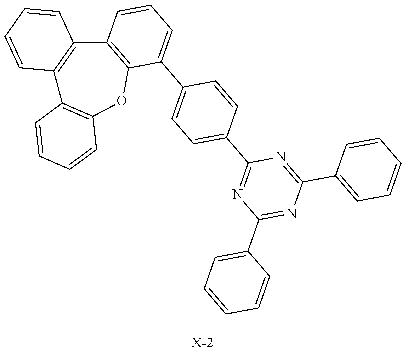 |
| | 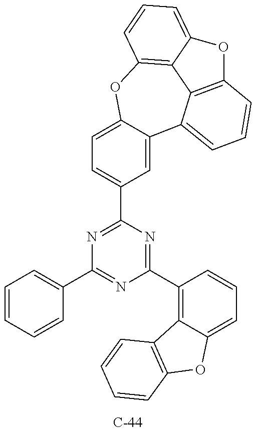 |
| | 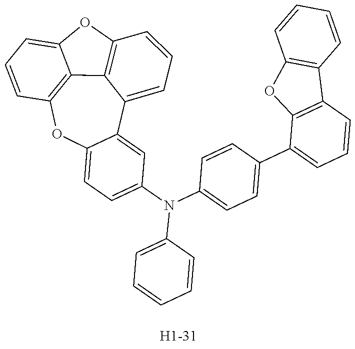 [SMILES: c1ccc(N(c2ccc(-c3cccc4c3oc3ccccc34)cc2)c2ccc3oc4cccc5oc6cccc(c3c2)c6c45)cc1] |
| | 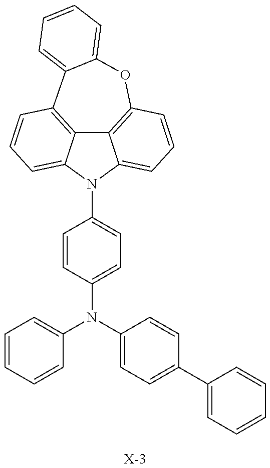 [SMILES: c1ccc(-c2ccc(N(c3ccccc3)c3ccc(-n4c5cccc6oc7ccccc7c7cccc4c7c65)cc3)cc2)cc1 ; "X-3"] |
| | 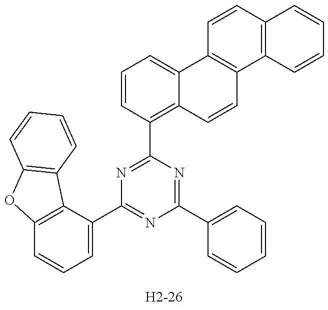 [SMILES: c1ccc(-c2nc(-c3cccc4c3ccc3c5ccccc5ccc43)nc(-c3cccc4oc5ccccc5c34)n2)cc1] |
| | 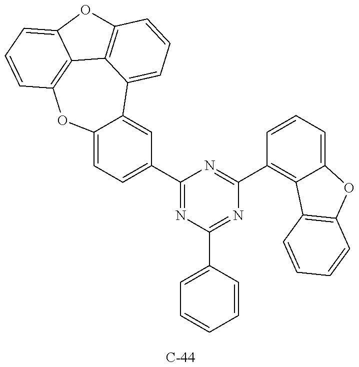 |
| | 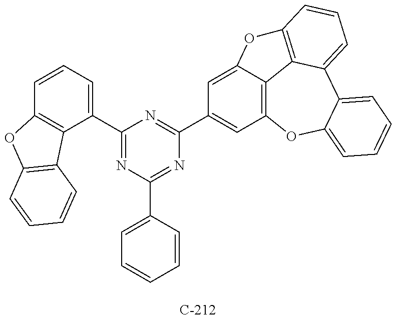 [SMILES: c1ccc(-c2nc(-c3cc4oc5ccccc5c5cccc6oc(c3)c4c65)nc(-c3cccc4oc5ccccc5c34)n2)cc1] |
| | 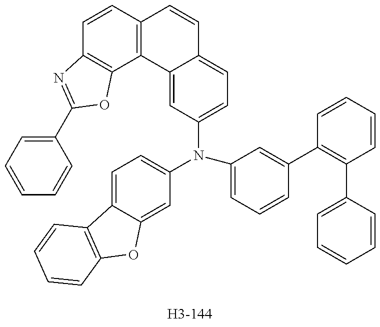 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1] |
| | 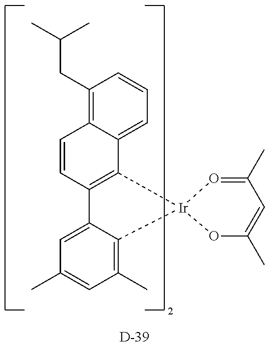 |
| Electron Transport Layer/ Electron Injection Layer | 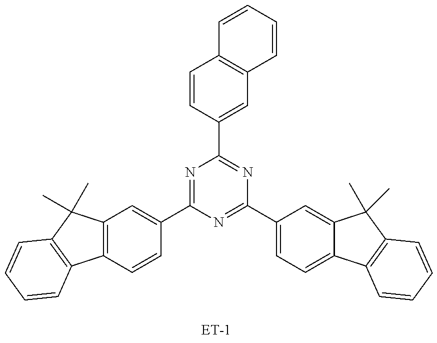 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 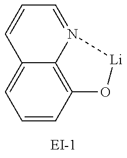 |

1. An organic electroluminescent compound represented by the following Formula 1: 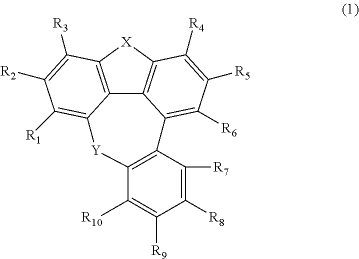
wherein in Formula 1,
X and Y, each independently, represent O or S;
R₁ to R₁₀, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), a substituted or unsubstituted mono- or di-(C1-C30)alkylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, or a substituted or unsubstituted (C6-C30)aryl(3- to 30-membered)heteroarylamino; or may be linked to an adjacent substituent(s) to form a ring(s); with the proviso that R₆ and R₇ are not linked to each other to form a ring(s);
with the proviso that at least one of R₁ to R₁₀ is represented by the following Formula 1-1 or 1-2: 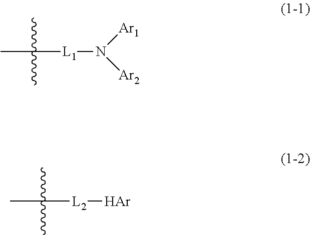
wherein in Formulas 1-1 and 1-2,
L₁ and L₂, each independently, represent a single bond, a substituted or unsubstituted (C6-C30)arylene, or a substituted or unsubstituted (3- to 30-membered)heteroarylene;
Ar₁ and Ar₂, each independently, represent hydrogen, deuterium, a substituted or unsubstituted (C6-C30)aryl, or a substituted or unsubstituted (3- to 30-membered)heteroaryl; and
HAr represents a substituted or unsubstituted (3- to 30-membered)heteroaryl containing one or more nitrogen atoms.

2. The organic electroluminescent compound according to claim 1, wherein the substituted alkyl, the substituted aryl, the substituted arylene, the substituted heteroaryl, the substituted heteroarylene, the substituted cycloalkyl, the substituted alkoxy, the substituted trialkylsilyl, the substituted dialkylarylsilyl, the substituted alkyldiarylsilyl, the substituted triarylsilyl, the substituted fused ring group of an aliphatic ring(s) and an aromatic ring(s), the substituted mono- or di-alkylamino, the substituted mono- or di-arylamino, the substituted alkylarylamino, the substituted mono- or di-heteroarylamino, and the substituted arylheteroarylamino, each independently, is substituted with at least one selected from the group consisting of deuterium, a halogen, a cyano, a carboxyl, a nitro, a hydroxyl, a (C1-C30)alkyl, a halo(C1-C30)alkyl, a (C2-C30)alkenyl, a (C2-C30)alkynyl, a (C1-C30)alkoxy, a (C1-C30)alkylthio, a (C3-C30)cycloalkyl, a (C3-C30)cycloalkenyl, a (3- to 7-membered)heterocycloalkyl, a (C6-C30)aryloxy, a (C6-C30)arylthio, a (3- to 30-membered)heteroaryl, a (C6-C30)aryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, a fused ring group of a (C3-C30)aliphatic ring(s) and a (C6-C30)aromatic ring(s), an amino, a mono- or di-(C1-C30)alkylamino, a substituted or unsubstituted mono- or di-(C6-C30)arylamino, a (C1-C30)alkyl(C6-C30)arylamino, a mono- or di-(3- to 30-membered)heteroarylamino, a (C1-C30)alkyl(3- to 30-membered)heteroarylamino, a (C6-C30)aryl(3- to 30-membered)heteroarylamino, a (C1-C30)alkylcarbonyl, a (C1-C30)alkoxycarbonyl, a (C6-C30)arylcarbonyl, a (C6-C30)arylphosphinyl, a di(C6-C30)arylboronyl, a di(C1-C30)alkylboronyl, a (C1-C30)alkyl(C6-C30)arylboronyl, a (C6-C30)aryl(C1-C30)alkyl, a (C1-C30)alkyl(C6-C30)aryl, and a combination thereof.

3. The organic electroluminescent compound according to claim 1, wherein HAr in Formula 1-2 is represented by the following Formula 1-a or 1-b: 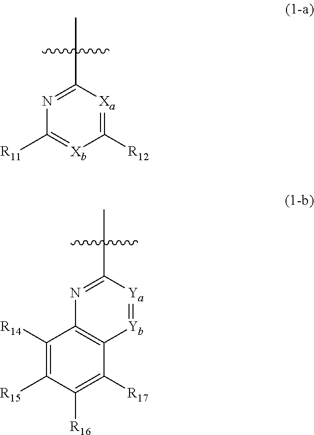
wherein in Formulas 1-a and 1-b,
Xₐ, X_{b}, Yₐ, and Y_{b}, each independently, represent N or CR₁₃, with the proviso that at least one of Xₐ and X_{b} represents N, and at least one of Yₐ and Y_{b} represents N; and
R₁₁ to R₁₇, each independently, represent hydrogen, deuterium, a halogen, a cyano, a substituted or unsubstituted (C1-C30)alkyl, a substituted or unsubstituted (C6-C50)aryl, a substituted or unsubstituted (3- to 50-membered)heteroaryl, a substituted or unsubstituted (C3-C30)cycloalkyl, a substituted or unsubstituted (3- to 7-membered)heterocycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent(s) to form a ring(s).

4. The organic electroluminescent compound according to claim 1, wherein Formula 1 is represented by any one of the following Formulas 2-1 to 2-10: 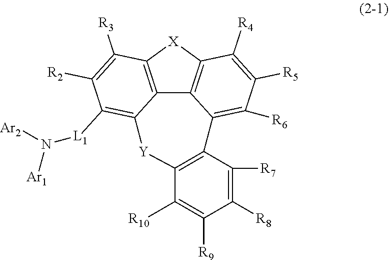 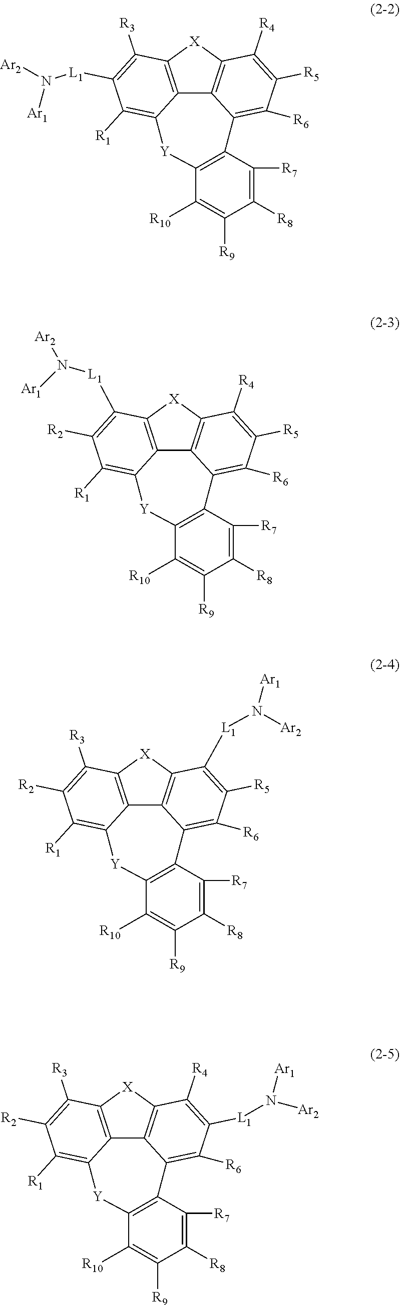 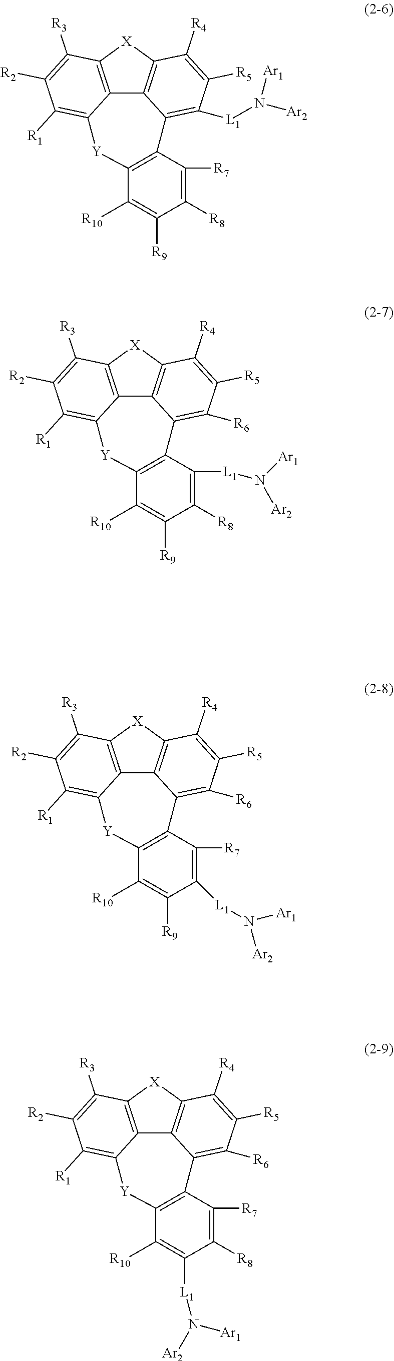 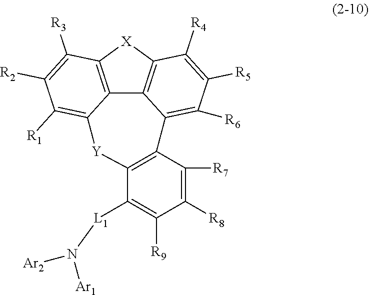
wherein in Formulas 2-1 to 2-10, X, Y, R₁ to R₁₀, L₁, Ar₁, and Ar₂ are as defined in claim 1.

5. The organic electroluminescent compound according to claim 1, wherein Formula 1 is represented by any one of the following Formulas 3-1 to 3-10: 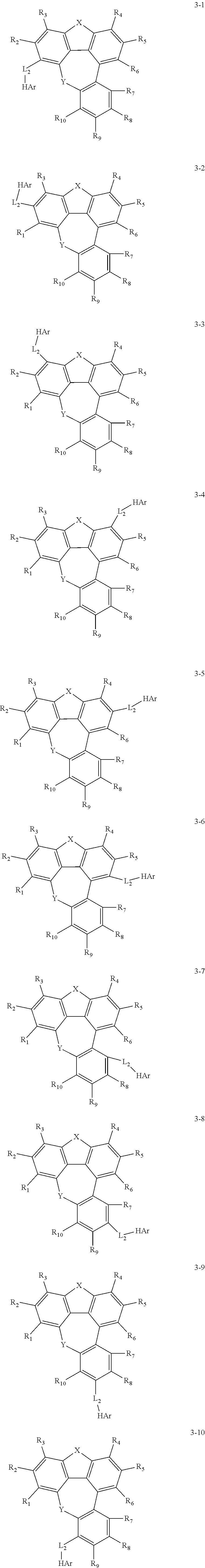
wherein in Formulas 3-1 to 3-10, X, Y, R₁ to R₁₀, L₂, and HAr are as defined in claim 1.

6. The organic electroluminescent compound according to claim 1, wherein the compound represented by Formula 1 is selected from the following compounds:

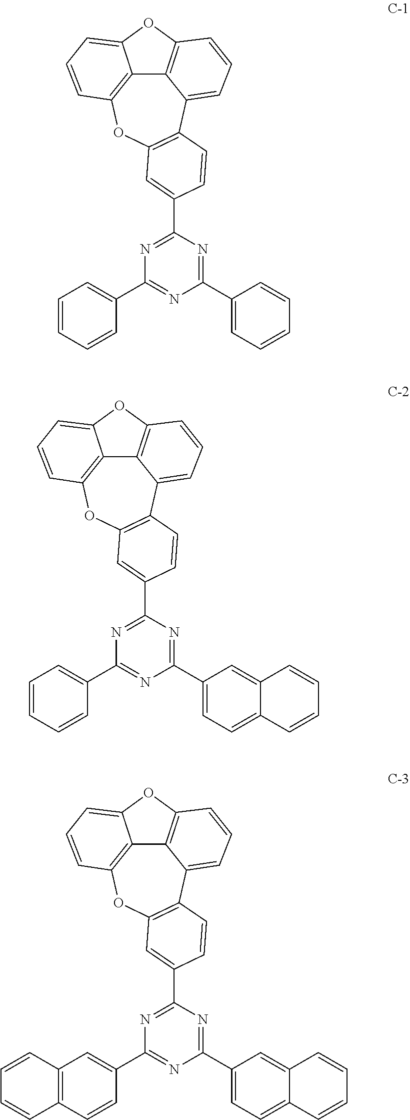 [SMILES: c1ccc(-c2nc(-c3ccccc3)nc(-c3ccc4c(c3)oc3cccc5oc6cccc4c6c35)n2)cc1 ; c1ccc(-c2nc(-c3ccc4ccccc4c3)nc(-c3ccc4c(c3)oc3cccc5oc6cccc4c6c35)n2)cc1 ; c1ccc2cc(-c3nc(-c4ccc5ccccc5c4)nc(-c4ccc5c(c4)oc4cccc6oc7cccc5c7c46)n3)ccc2c1]

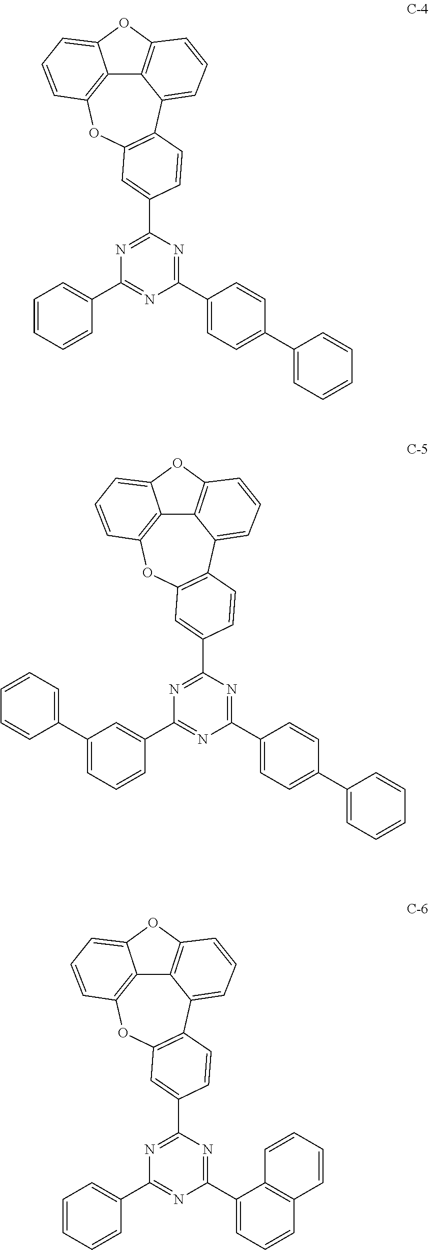 [SMILES: c1ccc(-c2ccc(-c3nc(-c4ccccc4)nc(-c4ccc5c(c4)oc4cccc6oc7cccc5c7c46)n3)cc2)cc1 ; c1ccc(-c2ccc(-c3nc(-c4cccc(-c5ccccc5)c4)nc(-c4ccc5c(c4)oc4cccc6oc7cccc5c7c46)n3)cc2)cc1 ; c1ccc(-c2nc(-c3ccc4c(c3)oc3cccc5oc6cccc4c6c35)nc(-c3cccc4ccccc34)n2)cc1]

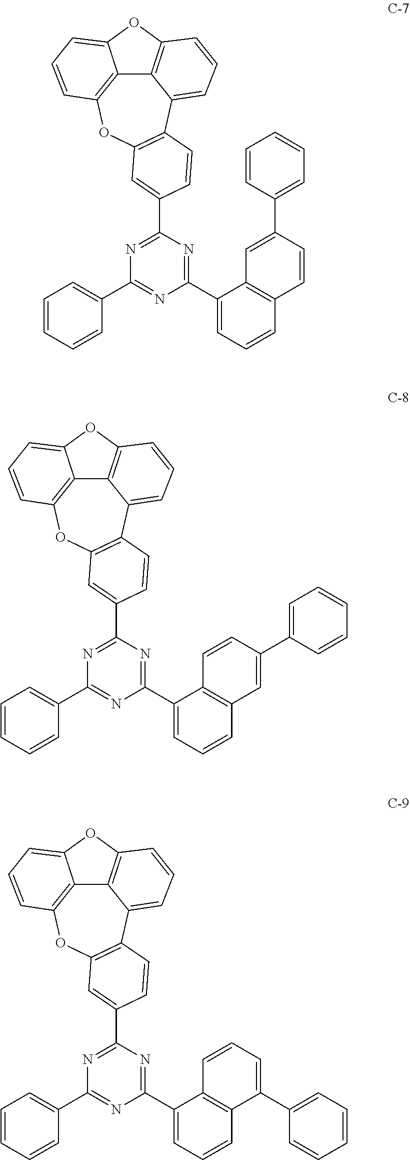 [SMILES: c1ccc(-c2ccc3cccc(-c4nc(-c5ccccc5)nc(-c5ccc6c(c5)oc5cccc7oc8cccc6c8c57)n4)c3c2)cc1 ; c1ccc(-c2ccc3c(-c4nc(-c5ccccc5)nc(-c5ccc6c(c5)oc5cccc7oc8cccc6c8c57)n4)cccc3c2)cc1 ; c1ccc(-c2nc(-c3ccc4c(c3)oc3cccc5oc6cccc4c6c35)nc(-c3cccc4c(-c5ccccc5)cccc34)n2)cc1]

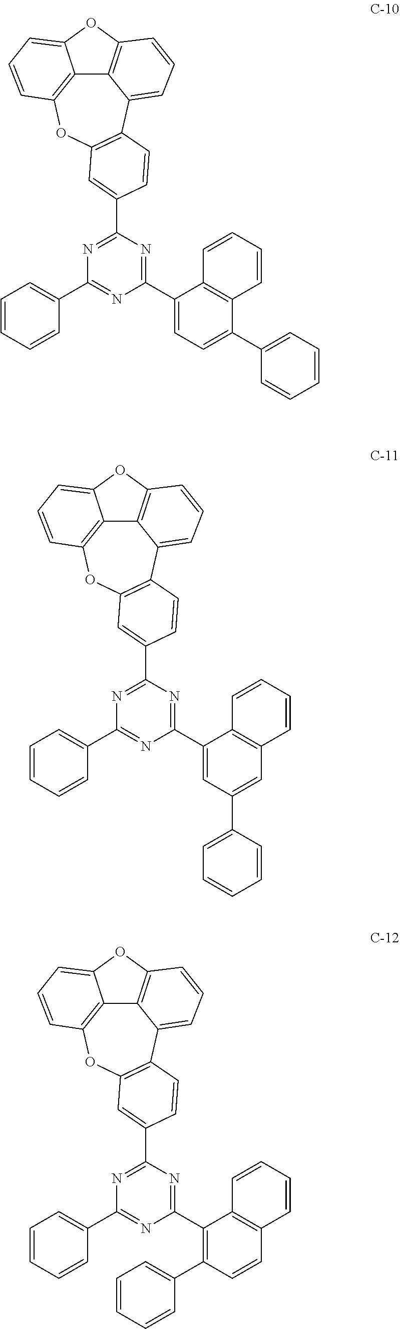 [SMILES: c1ccc(-c2nc(-c3ccc4c(c3)oc3cccc5oc6cccc4c6c35)nc(-c3ccc(-c4ccccc4)c4ccccc34)n2)cc1 ; c1ccc(-c2cc(-c3nc(-c4ccccc4)nc(-c4ccc5c(c4)oc4cccc6oc7cccc5c7c46)n3)c3ccccc3c2)cc1 ; c1ccc(-c2nc(-c3ccc4c(c3)oc3cccc5oc6cccc4c6c35)nc(-c3c(-c4ccccc4)ccc4ccccc34)n2)cc1]

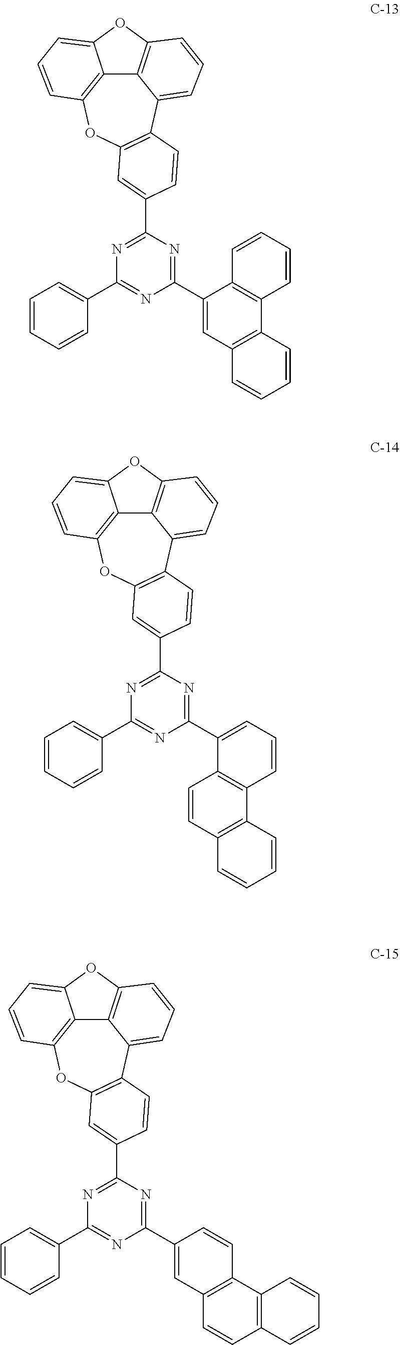 [SMILES: c1ccc(-c2nc(-c3ccc4c(c3)oc3cccc5oc6cccc4c6c35)nc(-c3cc4ccccc4c4ccccc34)n2)cc1 ; c1ccc(-c2nc(-c3ccc4c(c3)oc3cccc5oc6cccc4c6c35)nc(-c3cccc4c3ccc3ccccc34)n2)cc1 ; c1ccc(-c2nc(-c3ccc4c(ccc5ccccc54)c3)nc(-c3ccc4c(c3)oc3cccc5oc6cccc4c6c35)n2)cc1]

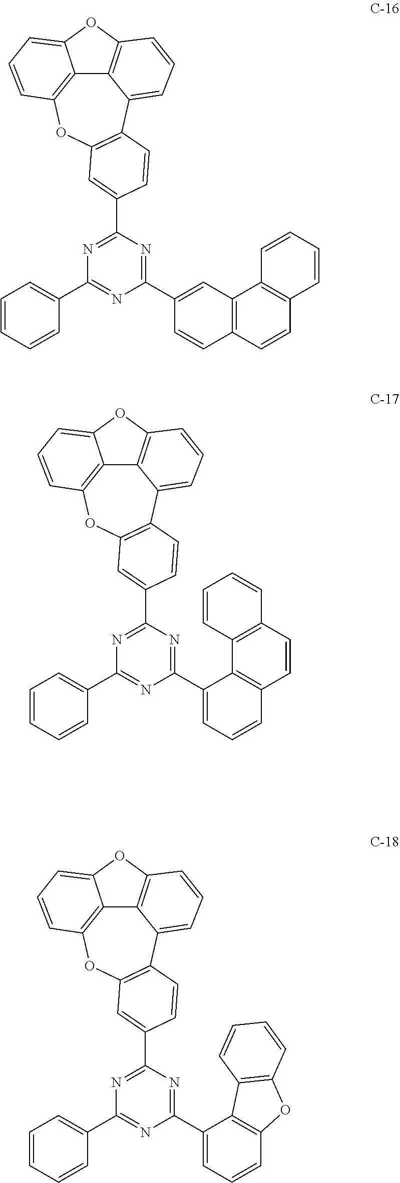 [SMILES: c1ccc(-c2nc(-c3ccc4c(c3)oc3cccc5oc6cccc4c6c35)nc(-c3ccc4ccc5ccccc5c4c3)n2)cc1 ; c1ccc(-c2nc(-c3ccc4c(c3)oc3cccc5oc6cccc4c6c35)nc(-c3cccc4ccc5ccccc5c34)n2)cc1 ; c1ccc(-c2nc(-c3ccc4c(c3)oc3cccc5oc6cccc4c6c35)nc(-c3cccc4oc5ccccc5c34)n2)cc1]

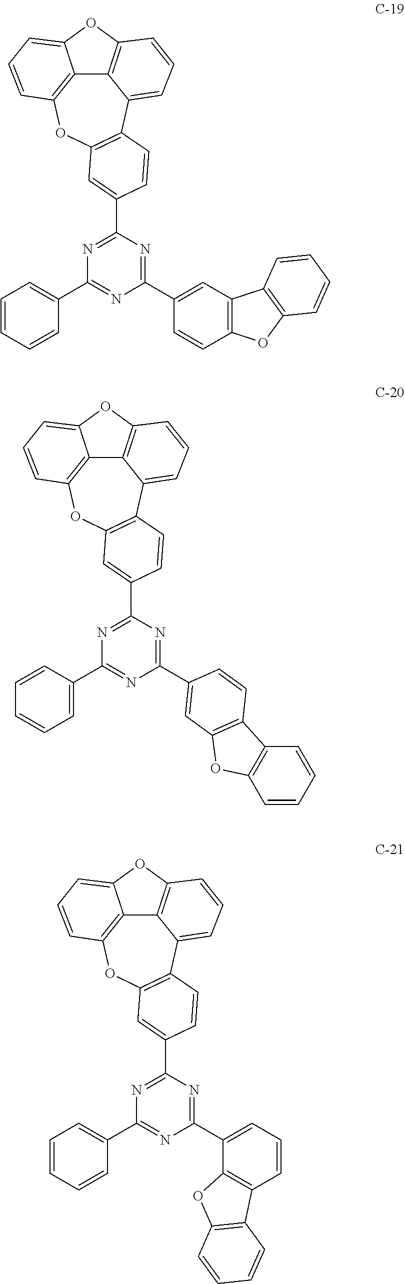 [SMILES: c1ccc(-c2nc(-c3ccc4c(c3)oc3cccc5oc6cccc4c6c35)nc(-c3ccc4oc5ccccc5c4c3)n2)cc1 ; c1ccc(-c2nc(-c3ccc4c(c3)oc3ccccc34)nc(-c3ccc4c(c3)oc3cccc5oc6cccc4c6c35)n2)cc1 ; c1ccc(-c2nc(-c3ccc4c(c3)oc3cccc5oc6cccc4c6c35)nc(-c3cccc4c3oc3ccccc34)n2)cc1]

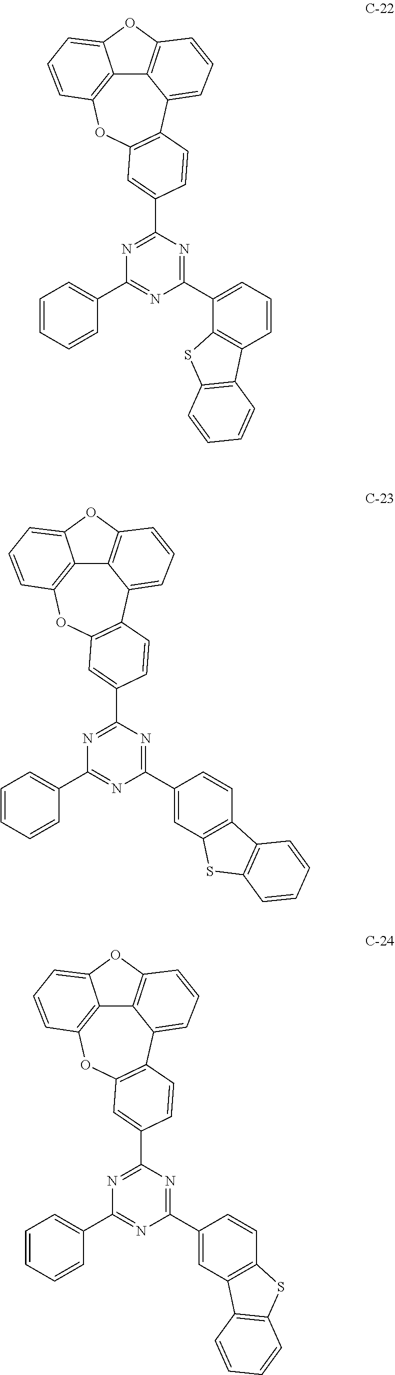 [SMILES: c1ccc(-c2nc(-c3ccc4c(c3)oc3cccc5oc6cccc4c6c35)nc(-c3cccc4c3sc3ccccc34)n2)cc1 ; c1ccc(-c2nc(-c3ccc4c(c3)oc3cccc5oc6cccc4c6c35)nc(-c3ccc4c(c3)sc3ccccc34)n2)cc1 ; c1ccc(-c2nc(-c3ccc4c(c3)oc3cccc5oc6cccc4c6c35)nc(-c3ccc4sc5ccccc5c4c3)n2)cc1]

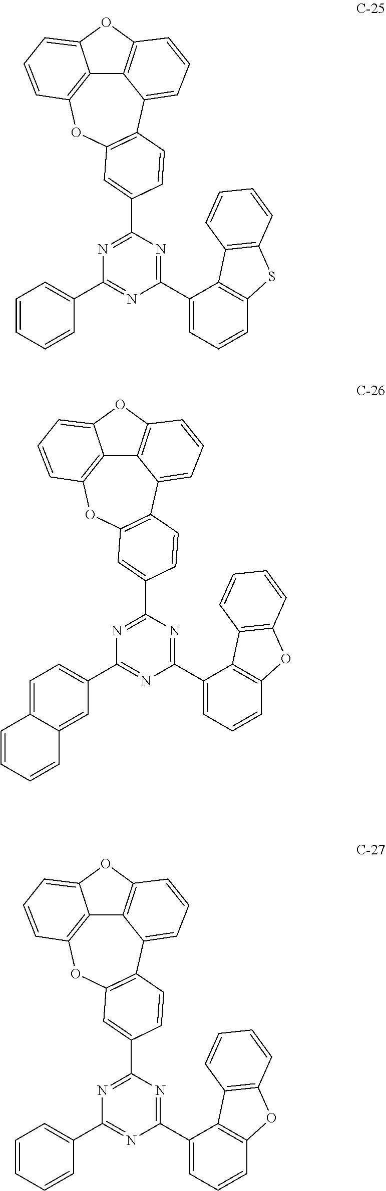 [SMILES: c1ccc(-c2nc(-c3ccc4c(c3)oc3cccc5oc6cccc4c6c35)nc(-c3cccc4sc5ccccc5c34)n2)cc1 ; c1ccc2cc(-c3nc(-c4ccc5c(c4)oc4cccc6oc7cccc5c7c46)nc(-c4cccc5oc6ccccc6c45)n3)ccc2c1 ; c1ccc(-c2nc(-c3ccc4c(c3)oc3cccc5oc6cccc4c6c35)nc(-c3cccc4oc5ccccc5c34)n2)cc1]

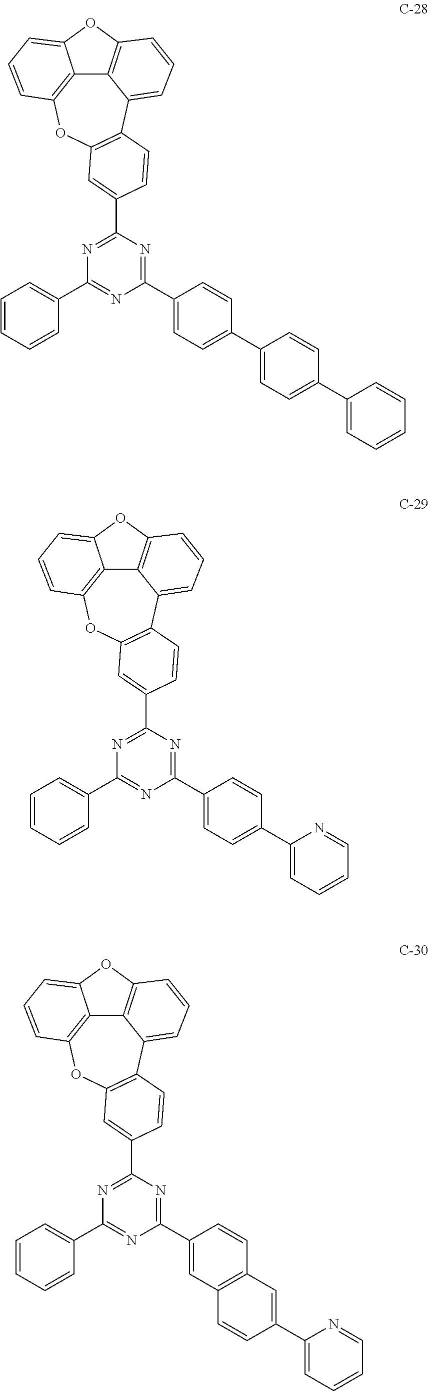 [SMILES: c1ccc(-c2ccc(-c3ccc(-c4nc(-c5ccccc5)nc(-c5ccc6c(c5)oc5cccc7oc8cccc6c8c57)n4)cc3)cc2)cc1 ; c1ccc(-c2nc(-c3ccc(-c4ccccn4)cc3)nc(-c3ccc4c(c3)oc3cccc5oc6cccc4c6c35)n2)cc1 ; c1ccc(-c2nc(-c3ccc4cc(-c5ccccn5)ccc4c3)nc(-c3ccc4c(c3)oc3cccc5oc6cccc4c6c35)n2)cc1]

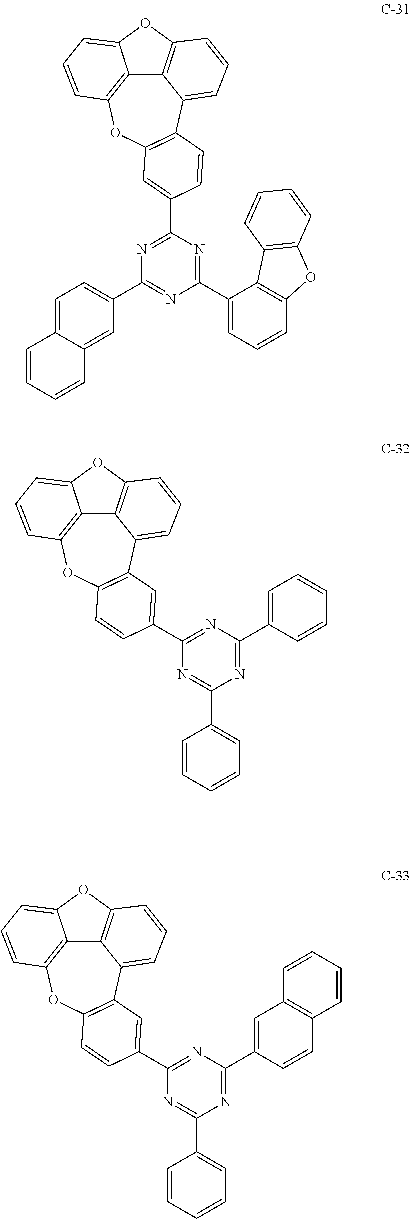 [SMILES: c1ccc2cc(-c3nc(-c4ccc5c(c4)oc4cccc6oc7cccc5c7c46)nc(-c4cccc5oc6ccccc6c45)n3)ccc2c1 ; c1ccc(-c2nc(-c3ccccc3)nc(-c3ccc4oc5cccc6oc7cccc(c4c3)c7c56)n2)cc1 ; c1ccc(-c2nc(-c3ccc4ccccc4c3)nc(-c3ccc4oc5cccc6oc7cccc(c4c3)c7c56)n2)cc1]

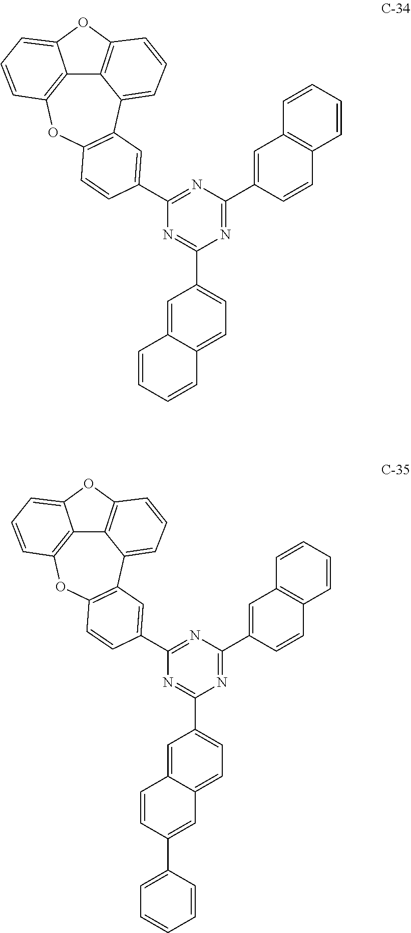 [SMILES: c1ccc2cc(-c3nc(-c4ccc5ccccc5c4)nc(-c4ccc5oc6cccc7oc8cccc(c5c4)c8c67)n3)ccc2c1 ; c1ccc(-c2ccc3cc(-c4nc(-c5ccc6ccccc6c5)nc(-c5ccc6oc7cccc8oc9cccc(c6c5)c9c78)n4)ccc3c2)cc1]

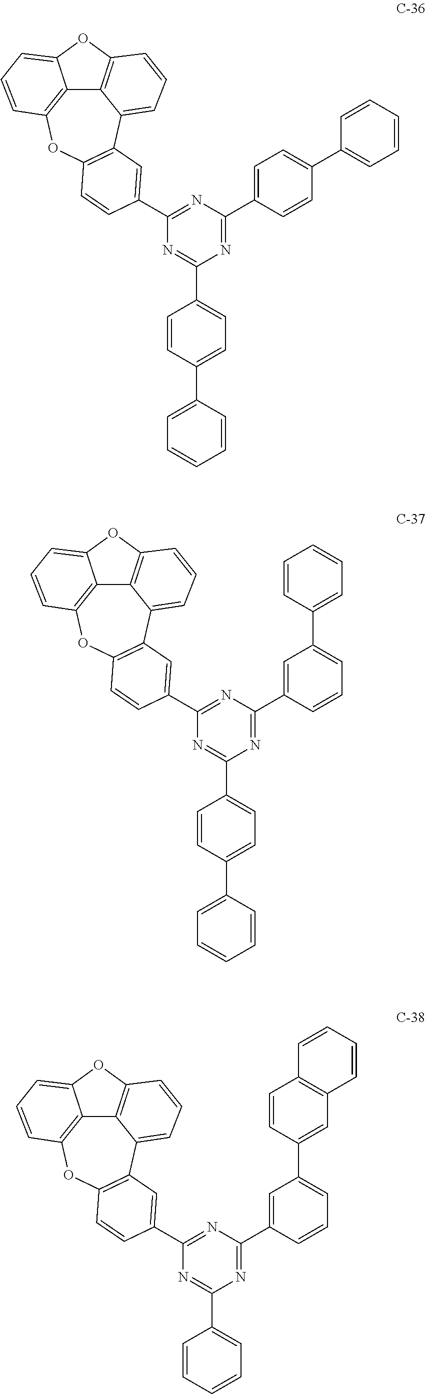 [SMILES: c1ccc(-c2ccc(-c3nc(-c4ccc(-c5ccccc5)cc4)nc(-c4ccc5oc6cccc7oc8cccc(c5c4)c8c67)n3)cc2)cc1 ; c1ccc(-c2ccc(-c3nc(-c4cccc(-c5ccccc5)c4)nc(-c4ccc5oc6cccc7oc8cccc(c5c4)c8c67)n3)cc2)cc1 ; c1ccc(-c2nc(-c3cccc(-c4ccc5ccccc5c4)c3)nc(-c3ccc4oc5cccc6oc7cccc(c4c3)c7c56)n2)cc1]

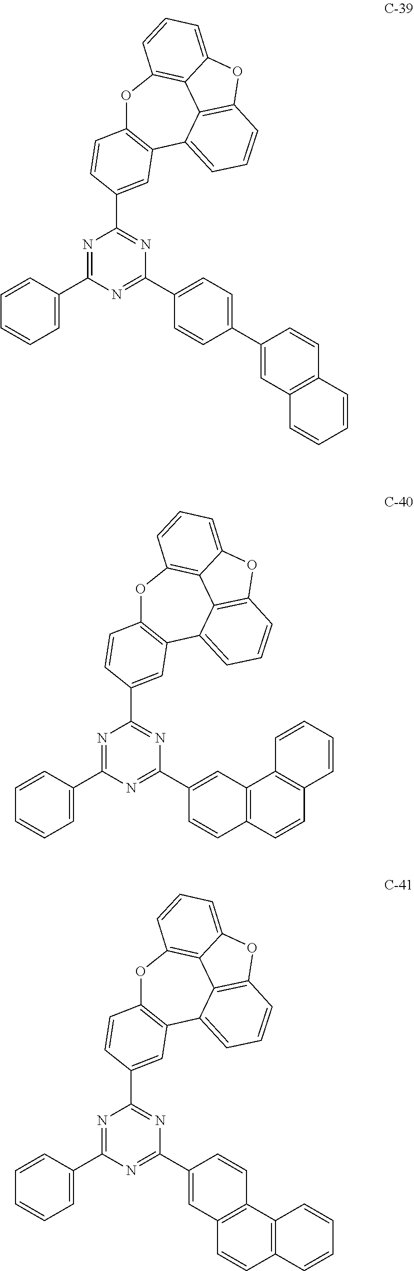 [SMILES: c1ccc(-c2nc(-c3ccc(-c4ccc5ccccc5c4)cc3)nc(-c3ccc4oc5cccc6oc7cccc(c4c3)c7c56)n2)cc1 ; c1ccc(-c2nc(-c3ccc4ccc5ccccc5c4c3)nc(-c3ccc4oc5cccc6oc7cccc(c4c3)c7c56)n2)cc1 ; c1ccc(-c2nc(-c3ccc4c(ccc5ccccc54)c3)nc(-c3ccc4oc5cccc6oc7cccc(c4c3)c7c56)n2)cc1]

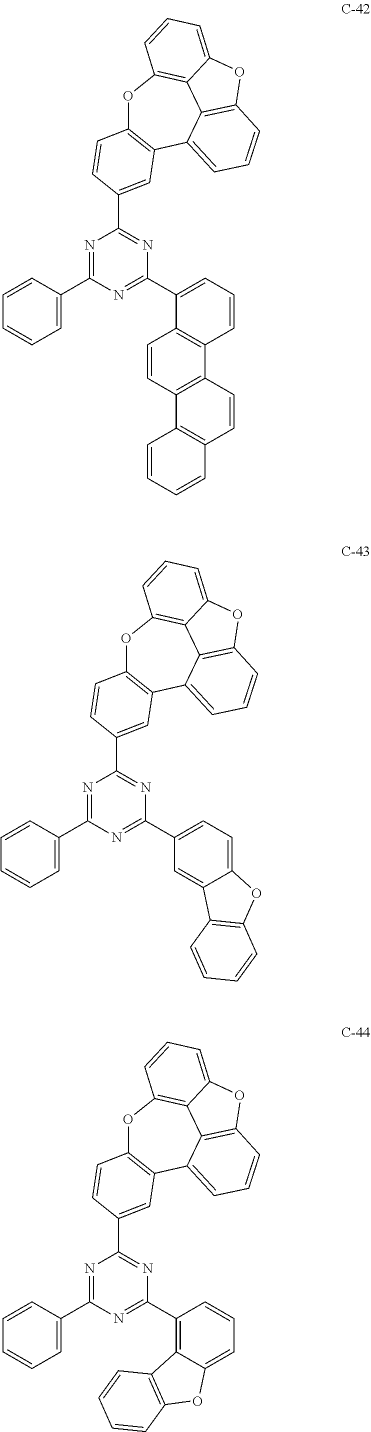 [SMILES: c1ccc(-c2nc(-c3ccc4oc5cccc6oc7cccc(c4c3)c7c56)nc(-c3cccc4c3ccc3c5ccccc5ccc43)n2)cc1 ; c1ccc(-c2nc(-c3ccc4oc5ccccc5c4c3)nc(-c3ccc4oc5cccc6oc7cccc(c4c3)c7c56)n2)cc1 ; c1ccc(-c2nc(-c3ccc4oc5cccc6oc7cccc(c4c3)c7c56)nc(-c3cccc4oc5ccccc5c34)n2)cc1]

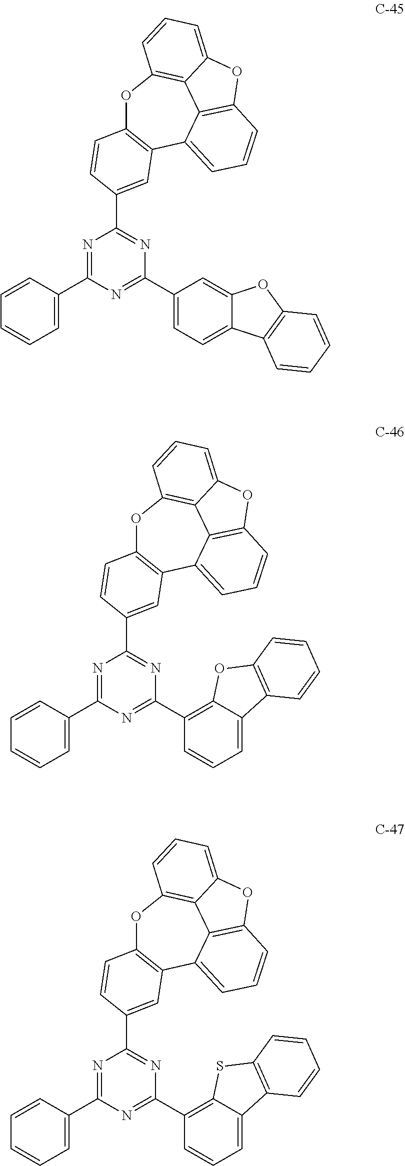 [SMILES: c1ccc(-c2nc(-c3ccc4c(c3)oc3ccccc34)nc(-c3ccc4oc5cccc6oc7cccc(c4c3)c7c56)n2)cc1 ; c1ccc(-c2nc(-c3ccc4oc5cccc6oc7cccc(c4c3)c7c56)nc(-c3cccc4c3oc3ccccc34)n2)cc1 ; c1ccc(-c2nc(-c3ccc4oc5cccc6oc7cccc(c4c3)c7c56)nc(-c3cccc4c3sc3ccccc34)n2)cc1]

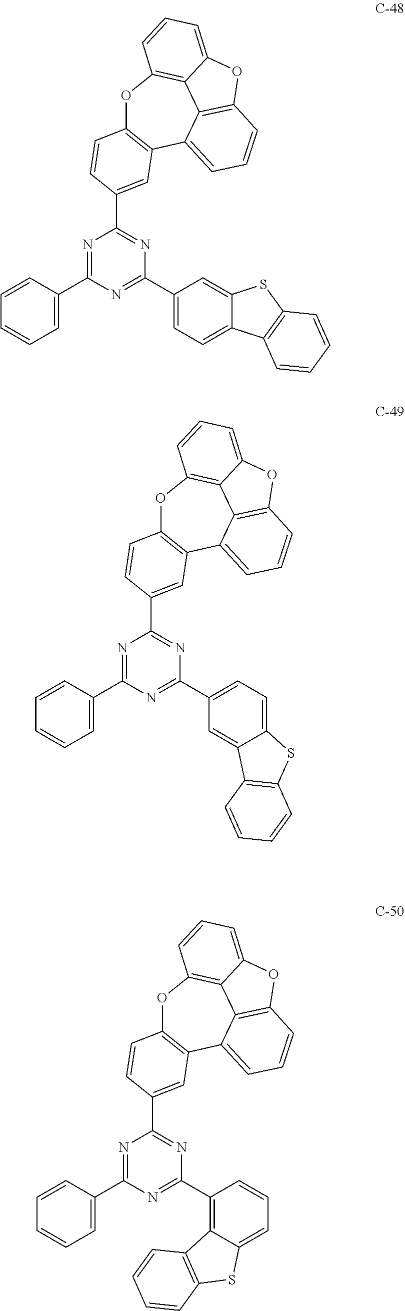 [SMILES: c1ccc(-c2nc(-c3ccc4c(c3)sc3ccccc34)nc(-c3ccc4oc5cccc6oc7cccc(c4c3)c7c56)n2)cc1 ; c1ccc(-c2nc(-c3ccc4oc5cccc6oc7cccc(c4c3)c7c56)nc(-c3ccc4sc5ccccc5c4c3)n2)cc1 ; c1ccc(-c2nc(-c3ccc4oc5cccc6oc7cccc(c4c3)c7c56)nc(-c3cccc4sc5ccccc5c34)n2)cc1]

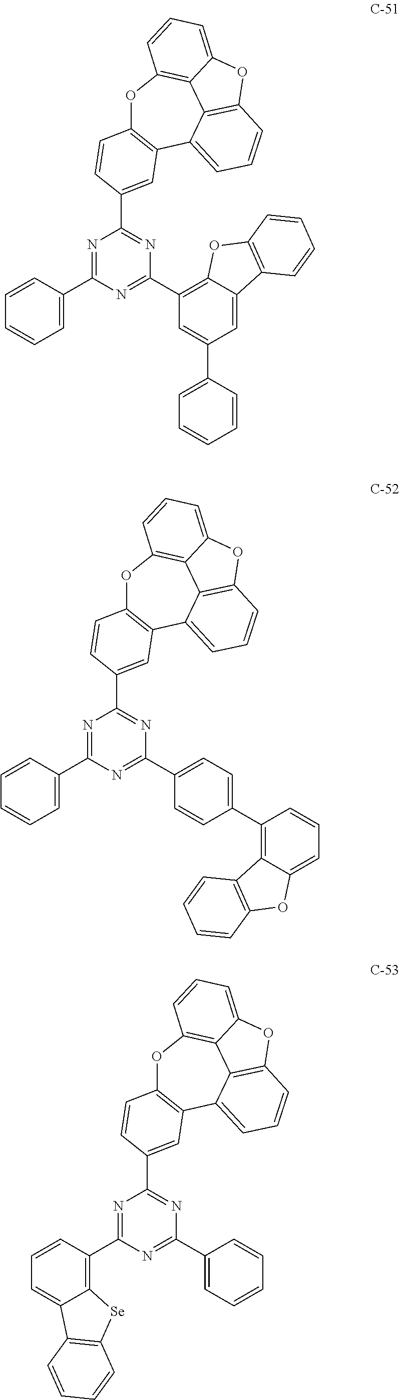

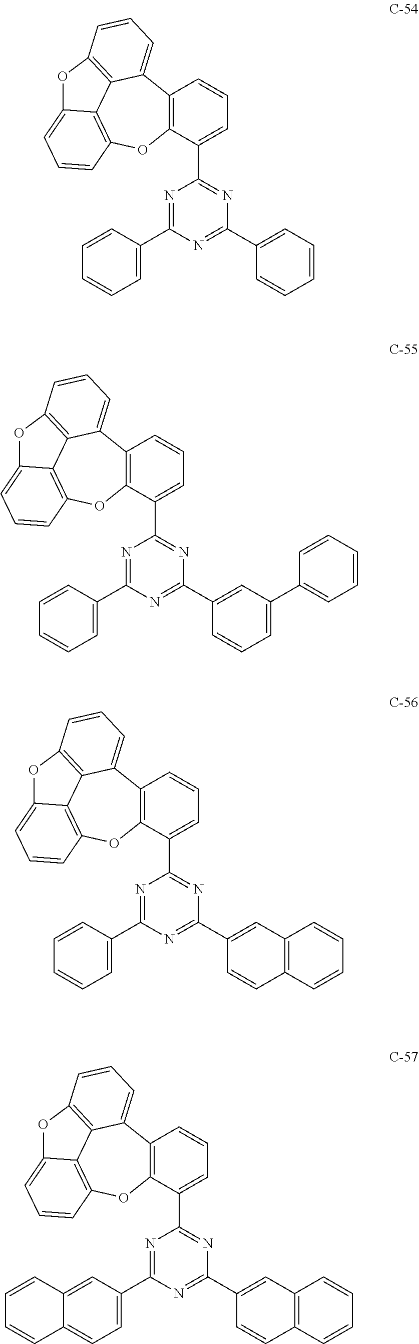 [SMILES: c1ccc(-c2nc(-c3ccccc3)nc(-c3cccc4c3oc3cccc5oc6cccc4c6c53)n2)cc1 ; c1ccc(-c2cccc(-c3nc(-c4ccccc4)nc(-c4cccc5c4oc4cccc6oc7cccc5c7c64)n3)c2)cc1 ; c1ccc(-c2nc(-c3ccc4ccccc4c3)nc(-c3cccc4c3oc3cccc5oc6cccc4c6c53)n2)cc1 ; c1ccc2cc(-c3nc(-c4ccc5ccccc5c4)nc(-c4cccc5c4oc4cccc6oc7cccc5c7c64)n3)ccc2c1]

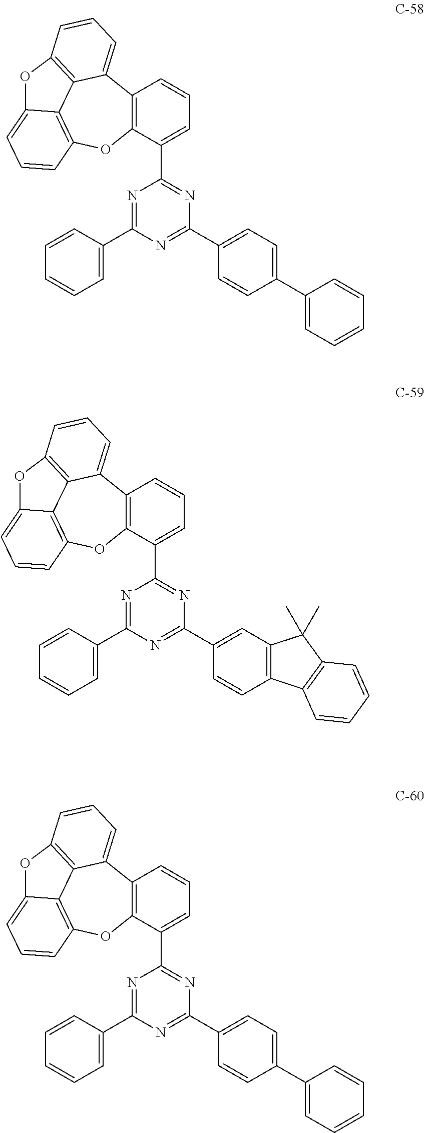 [SMILES: c1ccc(-c2ccc(-c3nc(-c4ccccc4)nc(-c4cccc5c4oc4cccc6oc7cccc5c7c64)n3)cc2)cc1 ; CC1(C)c2ccccc2-c2ccc(-c3nc(-c4ccccc4)nc(-c4cccc5c4oc4cccc6oc7cccc5c7c64)n3)cc21 ; c1ccc(-c2ccc(-c3nc(-c4ccccc4)nc(-c4cccc5c4oc4cccc6oc7cccc5c7c64)n3)cc2)cc1]

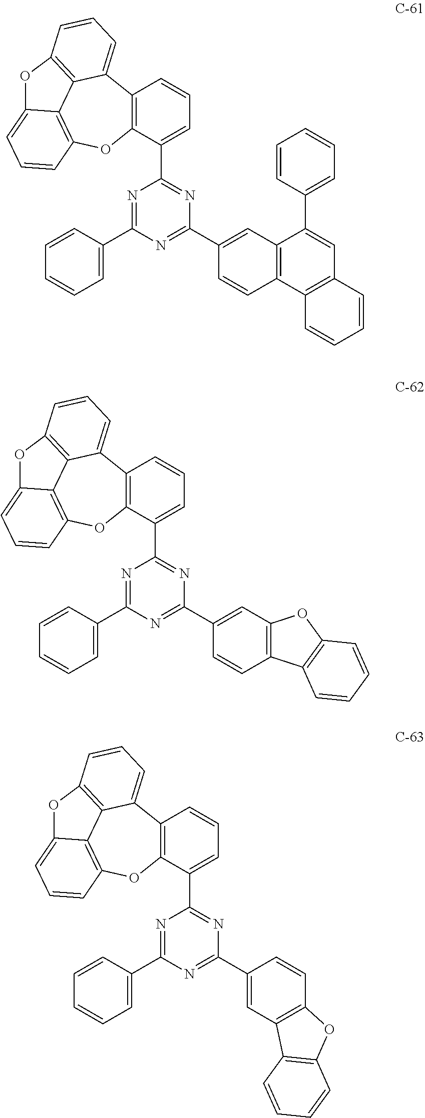 [SMILES: c1ccc(-c2nc(-c3ccc4c(c3)c(-c3ccccc3)cc3ccccc34)nc(-c3cccc4c3oc3cccc5oc6cccc4c6c53)n2)cc1 ; c1ccc(-c2nc(-c3ccc4c(c3)oc3ccccc34)nc(-c3cccc4c3oc3cccc5oc6cccc4c6c53)n2)cc1 ; c1ccc(-c2nc(-c3ccc4oc5ccccc5c4c3)nc(-c3cccc4c3oc3cccc5oc6cccc4c6c53)n2)cc1]

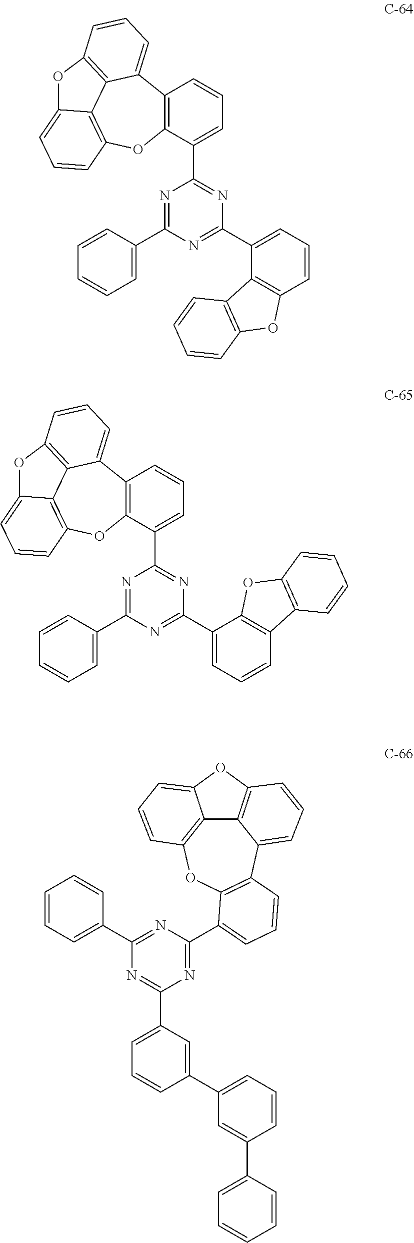 [SMILES: c1ccc(-c2nc(-c3cccc4c3oc3cccc5oc6cccc4c6c53)nc(-c3cccc4oc5ccccc5c34)n2)cc1 ; c1ccc(-c2nc(-c3cccc4c3oc3ccccc34)nc(-c3cccc4c3oc3cccc5oc6cccc4c6c53)n2)cc1 ; c1ccc(-c2cccc(-c3cccc(-c4nc(-c5ccccc5)nc(-c5cccc6c5oc5cccc7oc8cccc6c8c75)n4)c3)c2)cc1]

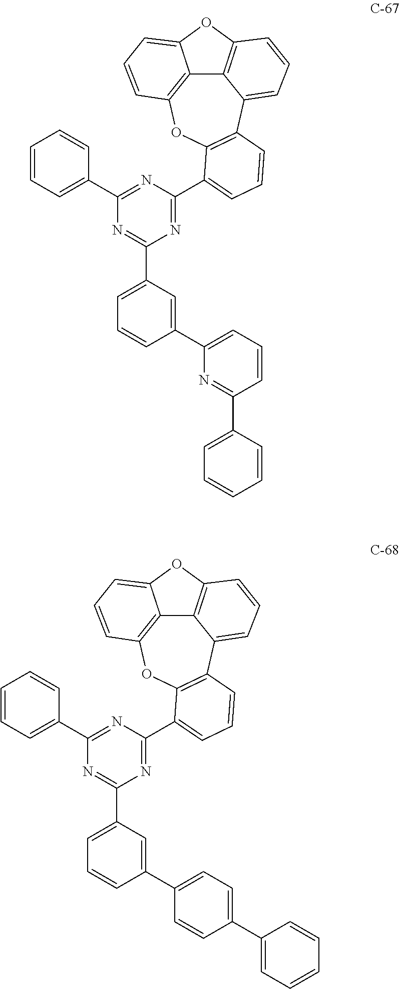 [SMILES: c1ccc(-c2cccc(-c3cccc(-c4nc(-c5ccccc5)nc(-c5cccc6c5oc5cccc7oc8cccc6c8c75)n4)c3)n2)cc1 ; c1ccc(-c2ccc(-c3cccc(-c4nc(-c5ccccc5)nc(-c5cccc6c5oc5cccc7oc8cccc6c8c75)n4)c3)cc2)cc1]

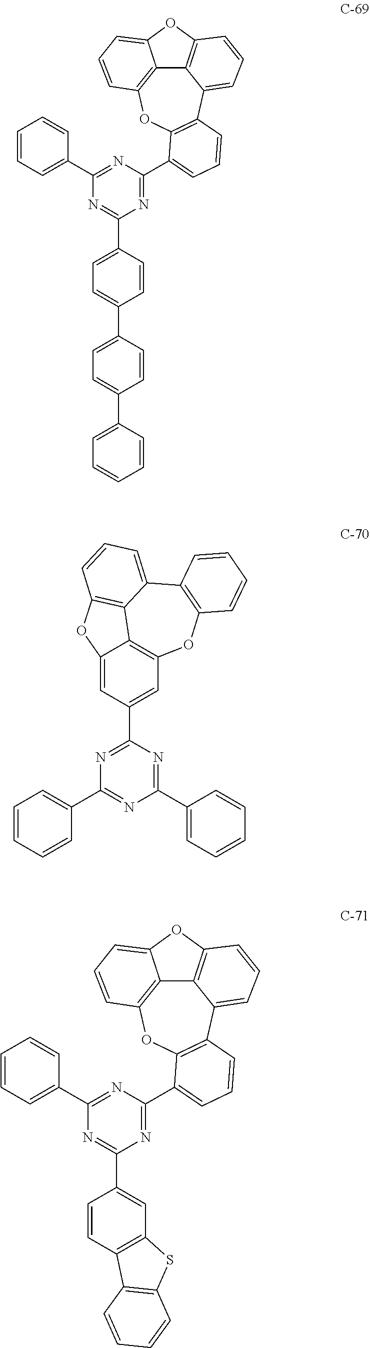 [SMILES: c1ccc(-c2ccc(-c3ccc(-c4nc(-c5ccccc5)nc(-c5cccc6c5oc5cccc7oc8cccc6c8c75)n4)cc3)cc2)cc1 ; c1ccc(-c2nc(-c3ccccc3)nc(-c3cc4oc5ccccc5c5cccc6oc(c3)c4c65)n2)cc1 ; c1ccc(-c2nc(-c3ccc4c(c3)sc3ccccc34)nc(-c3cccc4c3oc3cccc5oc6cccc4c6c53)n2)cc1]

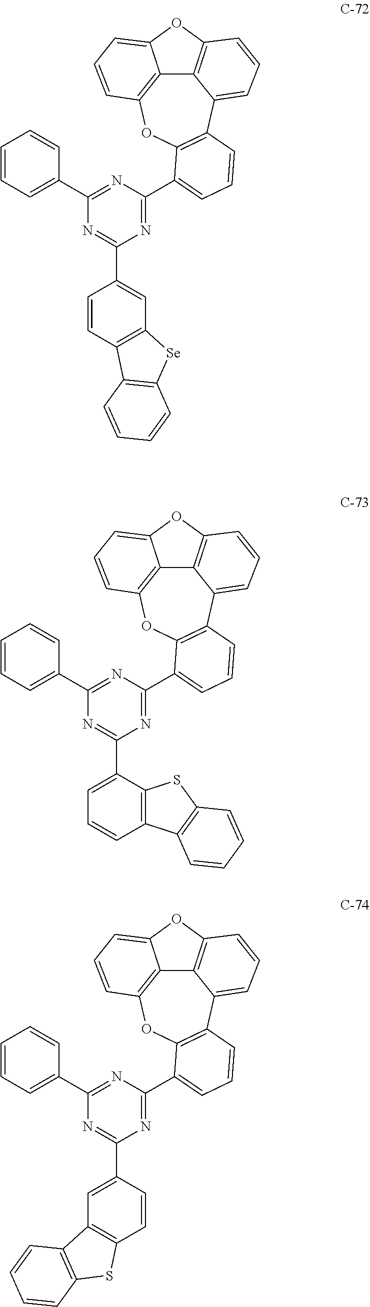 [SMILES: c1ccc(-c2nc(-c3ccc4c(c3)[se]c3ccccc34)nc(-c3cccc4c3oc3cccc5oc6cccc4c6c53)n2)cc1 ; c1ccc(-c2nc(-c3cccc4c3oc3cccc5oc6cccc4c6c53)nc(-c3cccc4c3sc3ccccc34)n2)cc1 ; c1ccc(-c2nc(-c3ccc4sc5ccccc5c4c3)nc(-c3cccc4c3oc3cccc5oc6cccc4c6c53)n2)cc1]

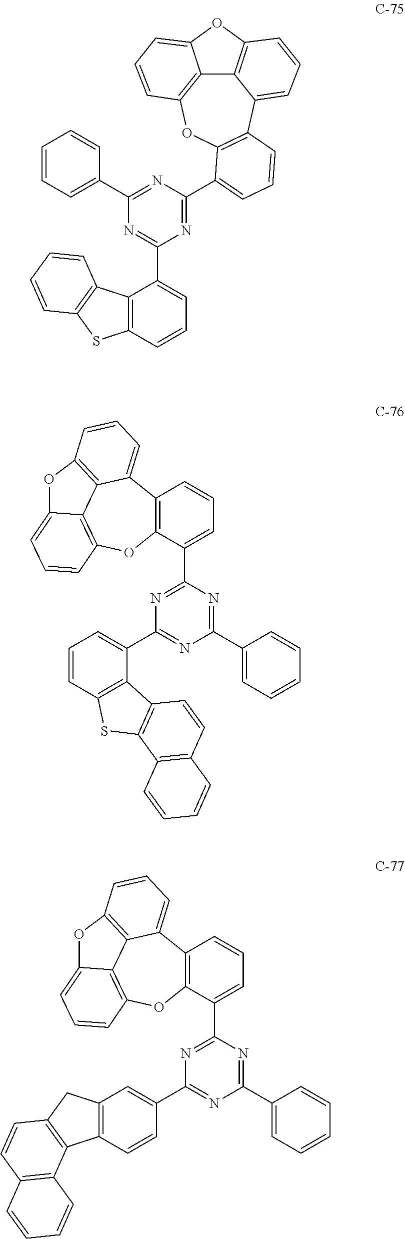 [SMILES: c1ccc(-c2nc(-c3cccc4c3oc3cccc5oc6cccc4c6c53)nc(-c3cccc4sc5ccccc5c34)n2)cc1 ; c1ccc(-c2nc(-c3cccc4c3oc3cccc5oc6cccc4c6c53)nc(-c3cccc4sc5c6ccccc6ccc5c34)n2)cc1 ; c1ccc(-c2nc(-c3ccc4c(c3)Cc3ccc5ccccc5c3-4)nc(-c3cccc4c3oc3cccc5oc6cccc4c6c53)n2)cc1]

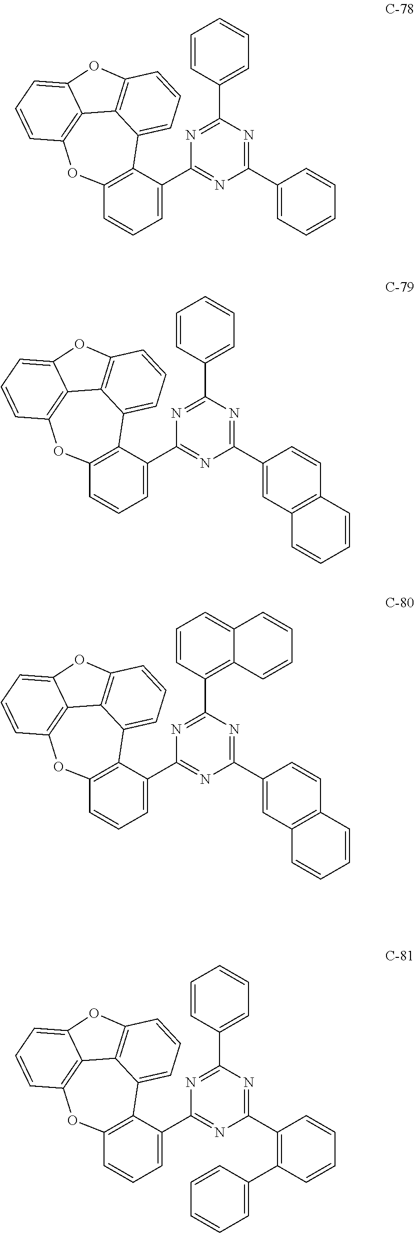 [SMILES: c1ccc(-c2nc(-c3ccccc3)nc(-c3cccc4oc5cccc6oc7cccc(c34)c7c56)n2)cc1 ; c1ccc(-c2nc(-c3ccc4ccccc4c3)nc(-c3cccc4oc5cccc6oc7cccc(c34)c7c56)n2)cc1 ; c1ccc2cc(-c3nc(-c4cccc5ccccc45)nc(-c4cccc5oc6cccc7oc8cccc(c45)c8c67)n3)ccc2c1 ; c1ccc(-c2nc(-c3ccccc3-c3ccccc3)nc(-c3cccc4oc5cccc6oc7cccc(c34)c7c56)n2)cc1]

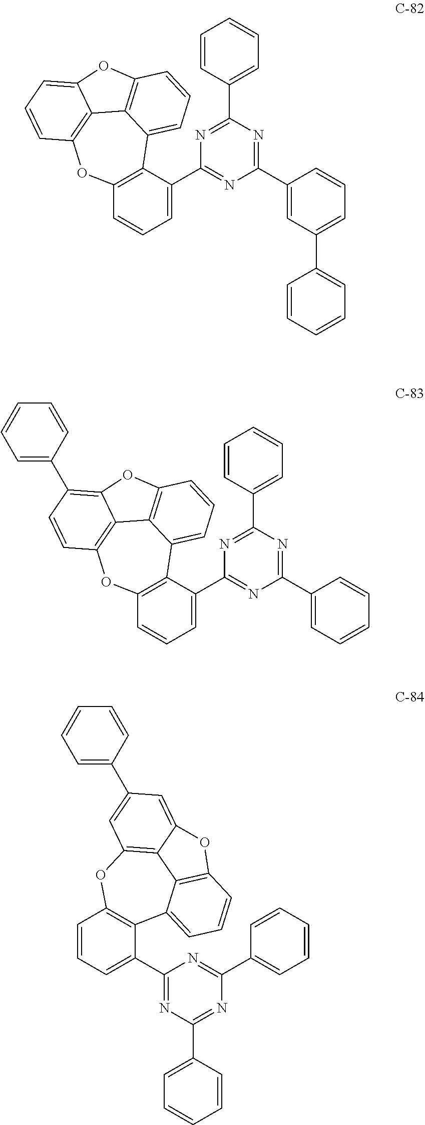 [SMILES: c1ccc(-c2cccc(-c3nc(-c4ccccc4)nc(-c4cccc5oc6cccc7oc8cccc(c45)c8c67)n3)c2)cc1 ; c1ccc(-c2nc(-c3ccccc3)nc(-c3cccc4oc5ccc(-c6ccccc6)c6oc7cccc(c34)c7c56)n2)cc1 ; c1ccc(-c2cc3oc4cccc(-c5nc(-c6ccccc6)nc(-c6ccccc6)n5)c4c4cccc5oc(c2)c3c54)cc1]

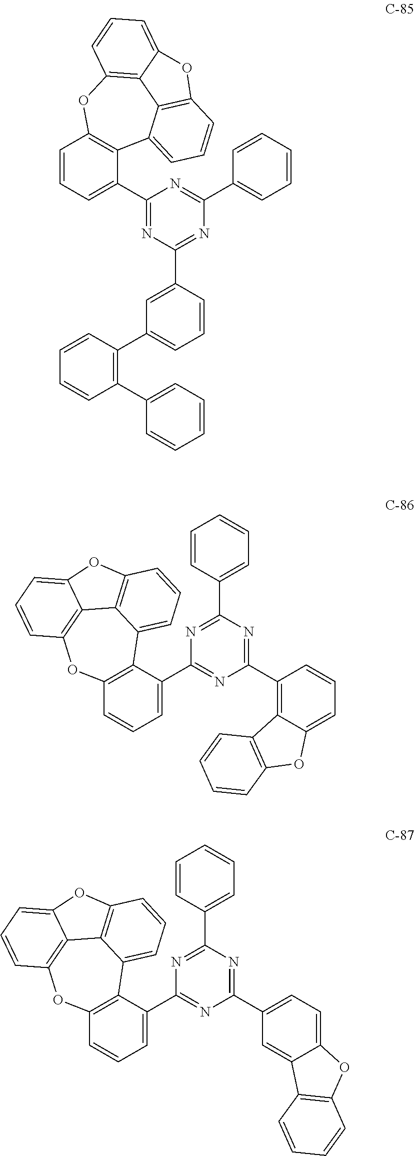 [SMILES: c1ccc(-c2nc(-c3cccc(-c4ccccc4-c4ccccc4)c3)nc(-c3cccc4oc5cccc6oc7cccc(c34)c7c56)n2)cc1 ; c1ccc(-c2nc(-c3cccc4oc5ccccc5c34)nc(-c3cccc4oc5cccc6oc7cccc(c34)c7c56)n2)cc1 ; c1ccc(-c2nc(-c3ccc4oc5ccccc5c4c3)nc(-c3cccc4oc5cccc6oc7cccc(c34)c7c56)n2)cc1]

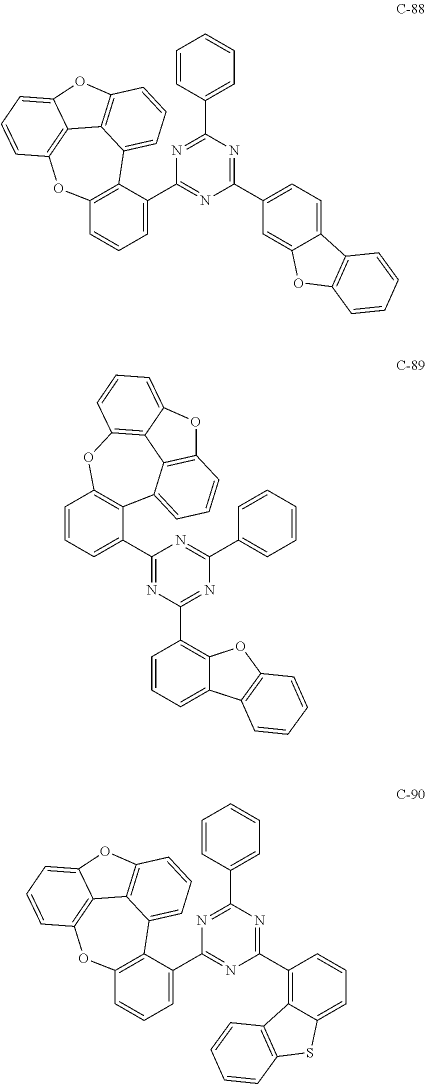 [SMILES: c1ccc(-c2nc(-c3ccc4c(c3)oc3ccccc34)nc(-c3cccc4oc5cccc6oc7cccc(c34)c7c56)n2)cc1 ; c1ccc(-c2nc(-c3cccc4c3oc3ccccc34)nc(-c3cccc4oc5cccc6oc7cccc(c34)c7c56)n2)cc1 ; c1ccc(-c2nc(-c3cccc4oc5cccc6oc7cccc(c34)c7c56)nc(-c3cccc4sc5ccccc5c34)n2)cc1]

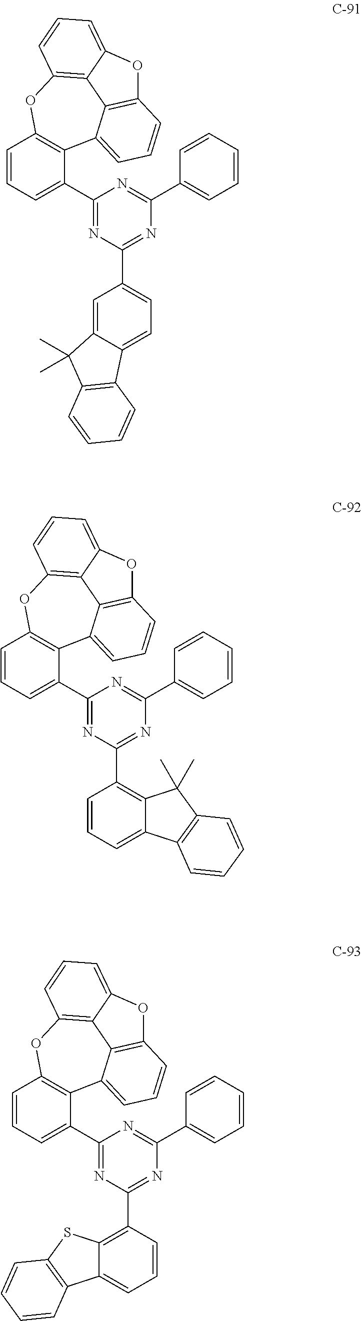 [SMILES: CC1(C)c2ccccc2-c2ccc(-c3nc(-c4ccccc4)nc(-c4cccc5oc6cccc7oc8cccc(c45)c8c67)n3)cc21 ; CC1(C)c2ccccc2-c2cccc(-c3nc(-c4ccccc4)nc(-c4cccc5oc6cccc7oc8cccc(c45)c8c67)n3)c21 ; c1ccc(-c2nc(-c3cccc4c3sc3ccccc34)nc(-c3cccc4oc5cccc6oc7cccc(c34)c7c56)n2)cc1]

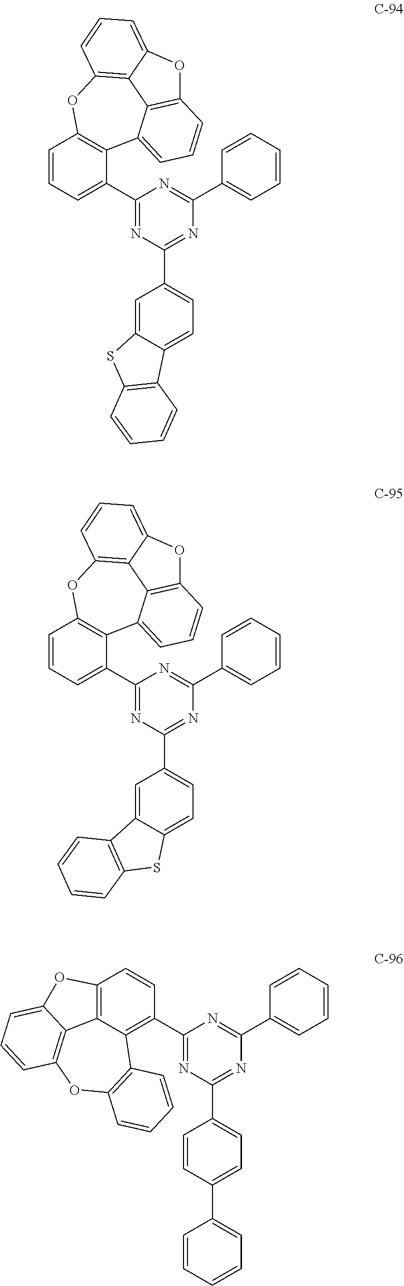 [SMILES: c1ccc(-c2nc(-c3ccc4c(c3)sc3ccccc34)nc(-c3cccc4oc5cccc6oc7cccc(c34)c7c56)n2)cc1 ; c1ccc(-c2nc(-c3ccc4sc5ccccc5c4c3)nc(-c3cccc4oc5cccc6oc7cccc(c34)c7c56)n2)cc1 ; c1ccc(-c2ccc(-c3nc(-c4ccccc4)nc(-c4ccc5oc6cccc7oc8ccccc8c4c5c76)n3)cc2)cc1]

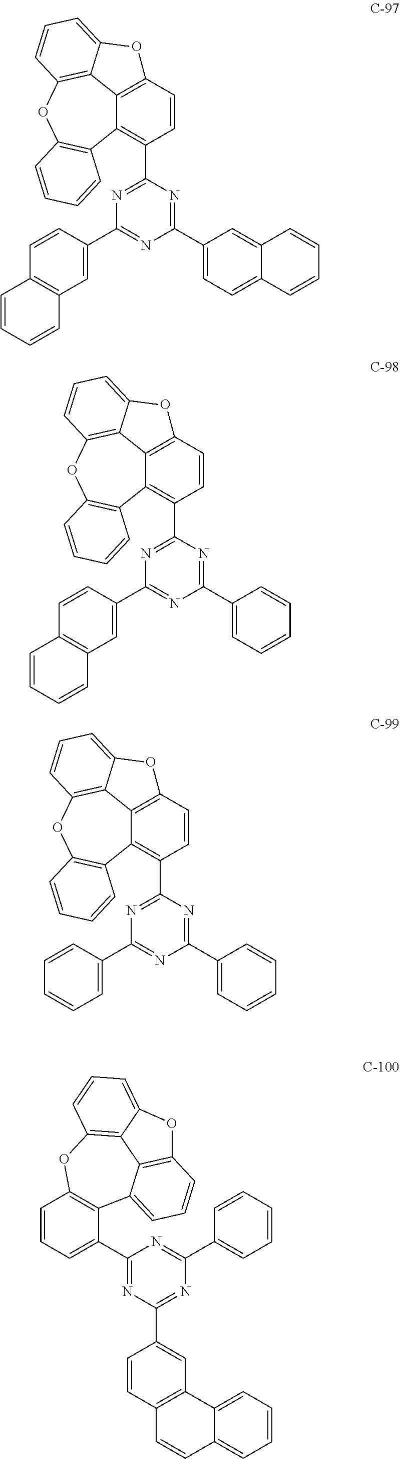

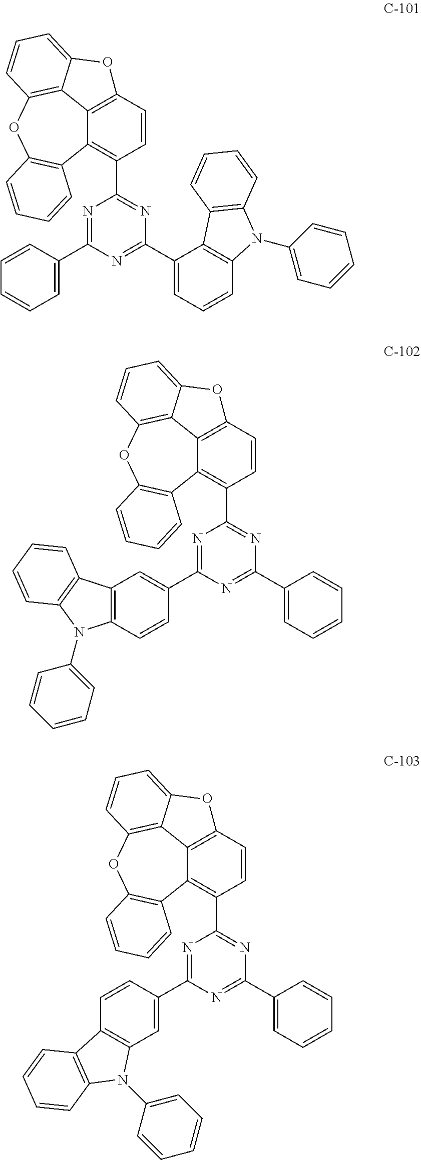 [SMILES: c1ccc(-c2nc(-c3ccc4oc5cccc6oc7ccccc7c3c4c65)nc(-c3cccc4c3c3ccccc3n4-c3ccccc3)n2)cc1 ; c1ccc(-c2nc(-c3ccc4c(c3)c3ccccc3n4-c3ccccc3)nc(-c3ccc4oc5cccc6oc7ccccc7c3c4c65)n2)cc1 ; c1ccc(-c2nc(-c3ccc4c5ccccc5n(-c5ccccc5)c4c3)nc(-c3ccc4oc5cccc6oc7ccccc7c3c4c65)n2)cc1]

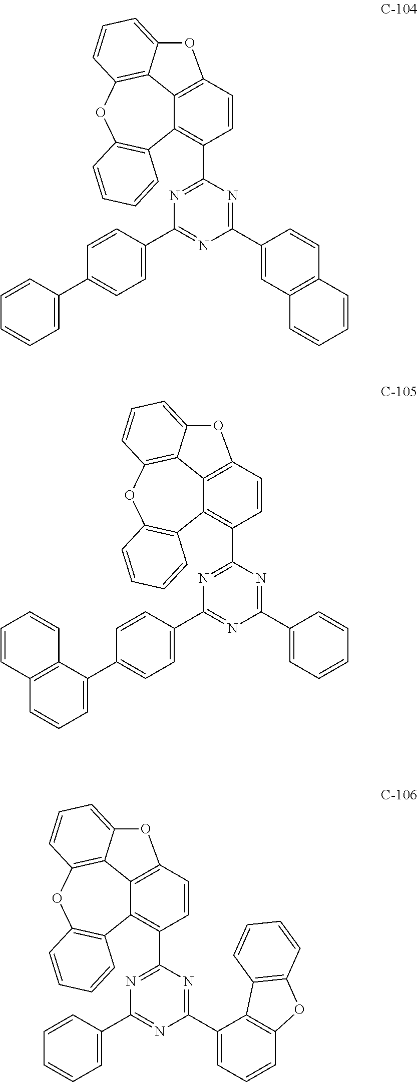 [SMILES: c1ccc(-c2ccc(-c3nc(-c4ccc5ccccc5c4)nc(-c4ccc5oc6cccc7oc8ccccc8c4c5c76)n3)cc2)cc1 ; c1ccc(-c2nc(-c3ccc(-c4cccc5ccccc45)cc3)nc(-c3ccc4oc5cccc6oc7ccccc7c3c4c65)n2)cc1 ; c1ccc(-c2nc(-c3cccc4oc5ccccc5c34)nc(-c3ccc4oc5cccc6oc7ccccc7c3c4c65)n2)cc1]

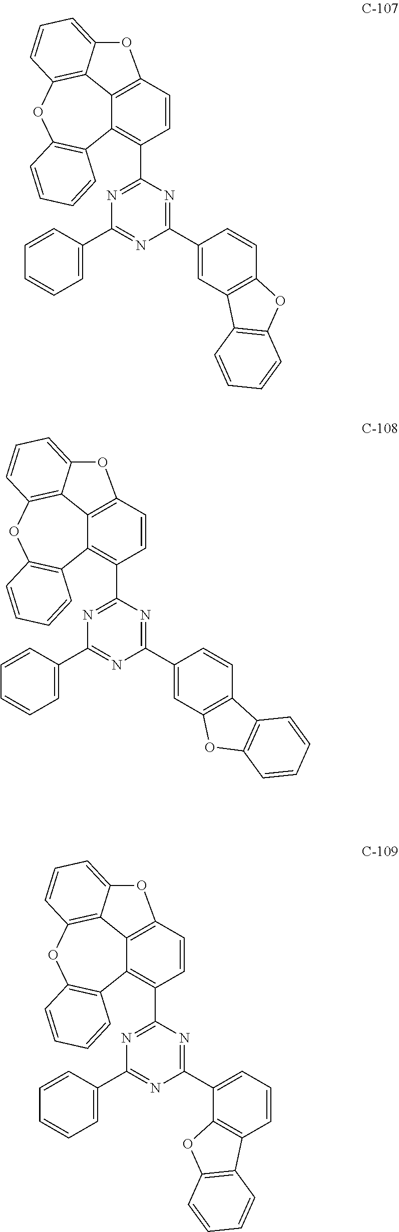 [SMILES: c1ccc(-c2nc(-c3ccc4oc5ccccc5c4c3)nc(-c3ccc4oc5cccc6oc7ccccc7c3c4c65)n2)cc1 ; c1ccc(-c2nc(-c3ccc4c(c3)oc3ccccc34)nc(-c3ccc4oc5cccc6oc7ccccc7c3c4c65)n2)cc1 ; c1ccc(-c2nc(-c3cccc4c3oc3ccccc34)nc(-c3ccc4oc5cccc6oc7ccccc7c3c4c65)n2)cc1]

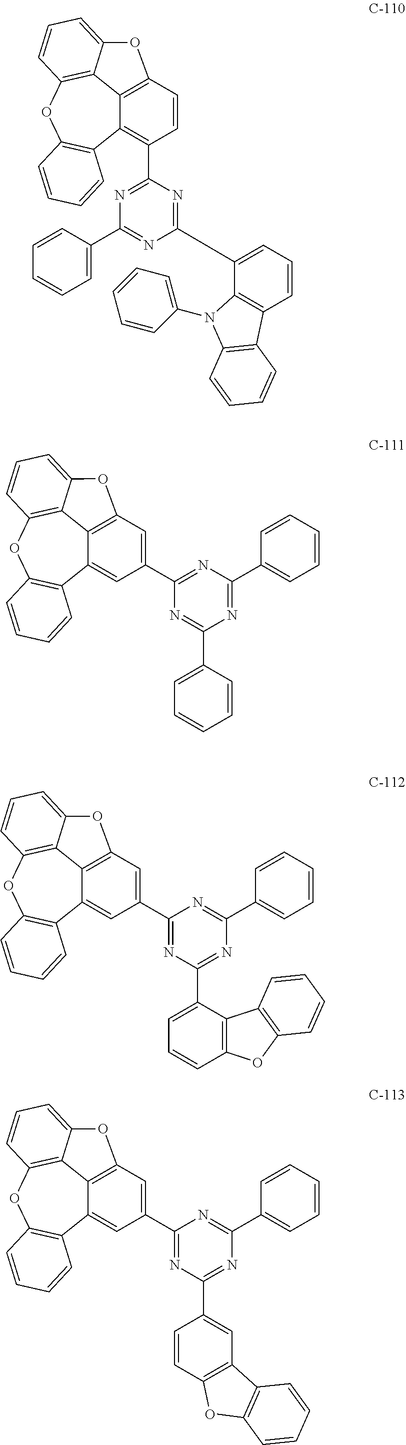 [SMILES: c1ccc(-c2nc(-c3ccc4oc5cccc6oc7ccccc7c3c4c65)nc(-c3cccc4c5ccccc5n(-c5ccccc5)c34)n2)cc1 ; c1ccc(-c2nc(-c3ccccc3)nc(-c3cc4oc5cccc6oc7ccccc7c(c3)c4c65)n2)cc1 ; c1ccc(-c2nc(-c3cc4oc5cccc6oc7ccccc7c(c3)c4c65)nc(-c3cccc4oc5ccccc5c34)n2)cc1 ; c1ccc(-c2nc(-c3ccc4oc5ccccc5c4c3)nc(-c3cc4oc5cccc6oc7ccccc7c(c3)c4c65)n2)cc1]

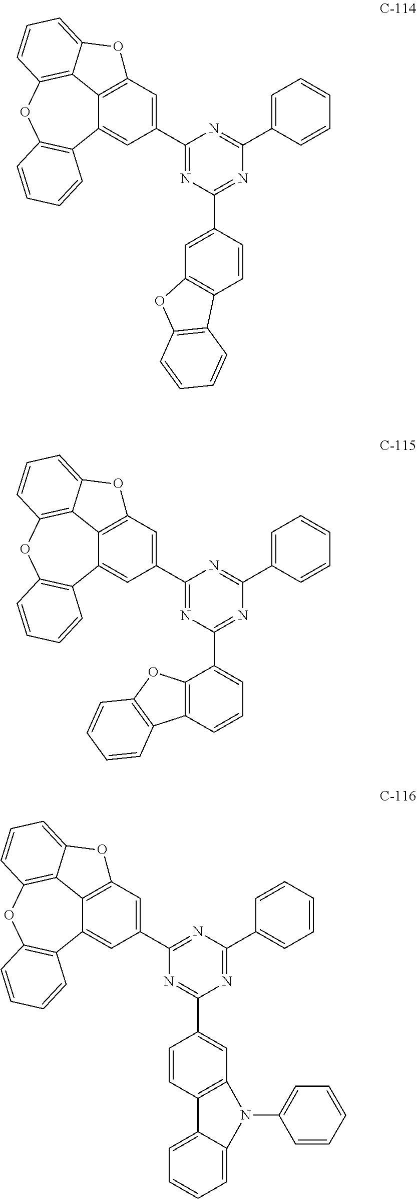 [SMILES: c1ccc(-c2nc(-c3ccc4c(c3)oc3ccccc34)nc(-c3cc4oc5cccc6oc7ccccc7c(c3)c4c65)n2)cc1 ; c1ccc(-c2nc(-c3cc4oc5cccc6oc7ccccc7c(c3)c4c65)nc(-c3cccc4c3oc3ccccc34)n2)cc1 ; c1ccc(-c2nc(-c3cc4oc5cccc6oc7ccccc7c(c3)c4c65)nc(-c3ccc4c5ccccc5n(-c5ccccc5)c4c3)n2)cc1]

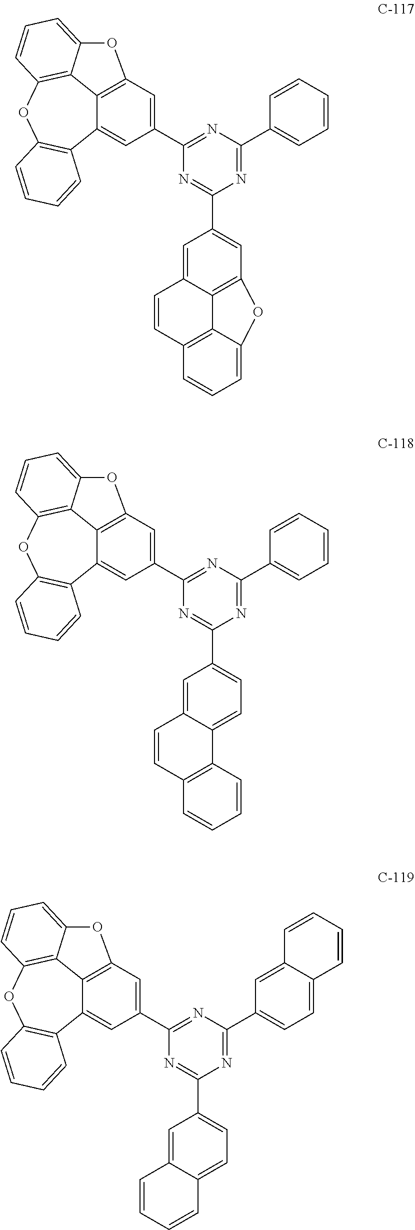 [SMILES: c1ccc(-c2nc(-c3cc4ccc5cccc6oc(c3)c4c56)nc(-c3cc4oc5cccc6oc7ccccc7c(c3)c4c65)n2)cc1 ; c1ccc(-c2nc(-c3ccc4c(ccc5ccccc54)c3)nc(-c3cc4oc5cccc6oc7ccccc7c(c3)c4c65)n2)cc1 ; c1ccc2cc(-c3nc(-c4ccc5ccccc5c4)nc(-c4cc5oc6cccc7oc8ccccc8c(c4)c5c76)n3)ccc2c1]

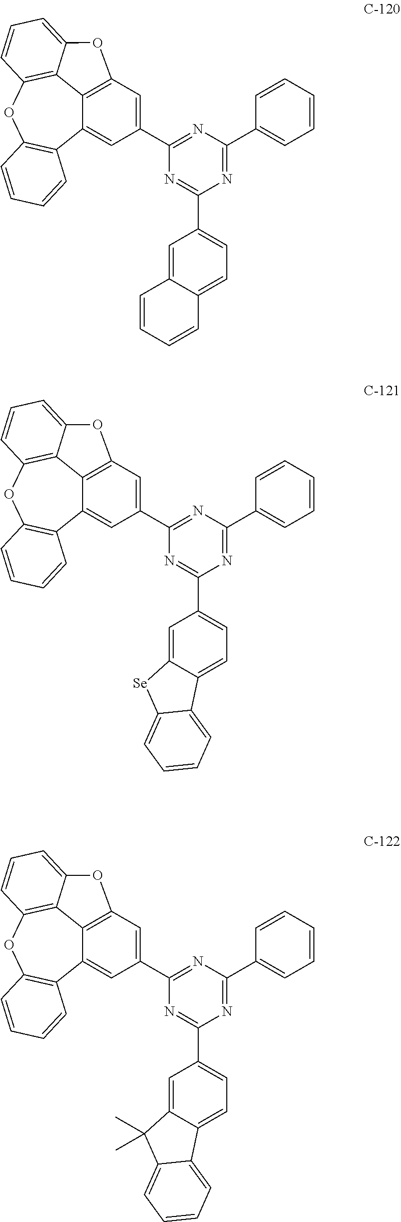 [SMILES: c1ccc(-c2nc(-c3ccc4ccccc4c3)nc(-c3cc4oc5cccc6oc7ccccc7c(c3)c4c65)n2)cc1 ; c1ccc(-c2nc(-c3ccc4c(c3)[se]c3ccccc34)nc(-c3cc4oc5cccc6oc7ccccc7c(c3)c4c65)n2)cc1 ; CC1(C)c2ccccc2-c2ccc(-c3nc(-c4ccccc4)nc(-c4cc5oc6cccc7oc8ccccc8c(c4)c5c76)n3)cc21]

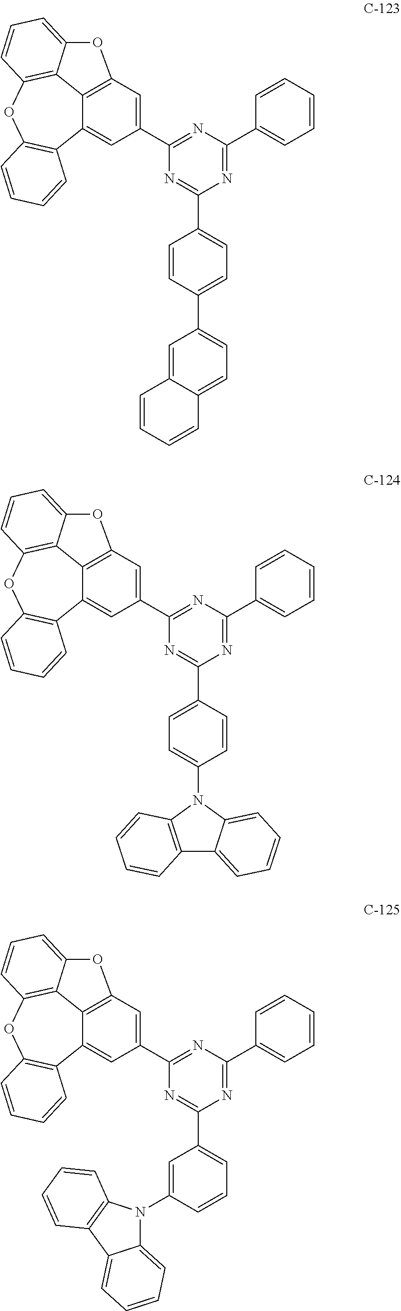 [SMILES: c1ccc(-c2nc(-c3ccc(-c4ccc5ccccc5c4)cc3)nc(-c3cc4oc5cccc6oc7ccccc7c(c3)c4c65)n2)cc1 ; c1ccc(-c2nc(-c3ccc(-n4c5ccccc5c5ccccc54)cc3)nc(-c3cc4oc5cccc6oc7ccccc7c(c3)c4c65)n2)cc1 ; c1ccc(-c2nc(-c3cccc(-n4c5ccccc5c5ccccc54)c3)nc(-c3cc4oc5cccc6oc7ccccc7c(c3)c4c65)n2)cc1]

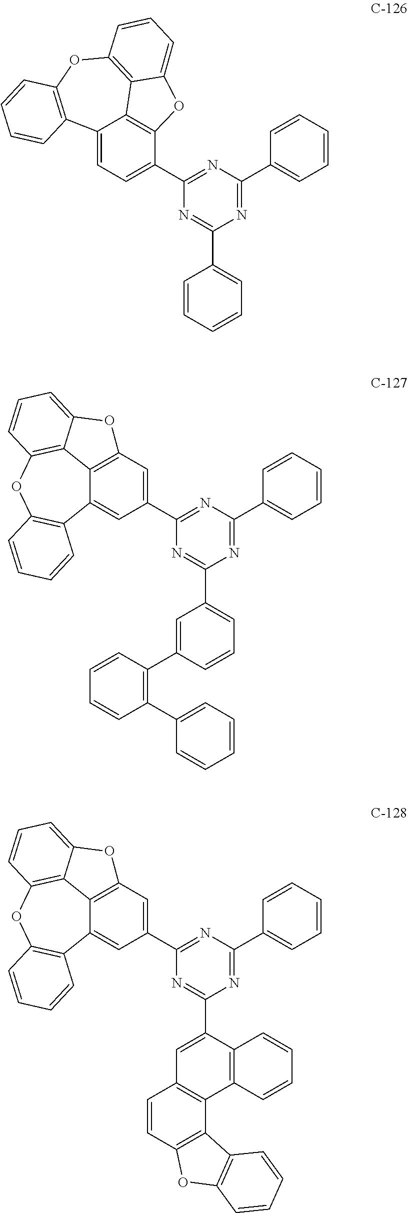 [SMILES: c1ccc(-c2nc(-c3ccccc3)nc(-c3ccc4c5ccccc5oc5cccc6oc3c4c56)n2)cc1 ; c1ccc(-c2nc(-c3cccc(-c4ccccc4-c4ccccc4)c3)nc(-c3cc4oc5cccc6oc7ccccc7c(c3)c4c65)n2)cc1 ; c1ccc(-c2nc(-c3cc4oc5cccc6oc7ccccc7c(c3)c4c65)nc(-c3cc4ccc5oc6ccccc6c5c4c4ccccc34)n2)cc1]

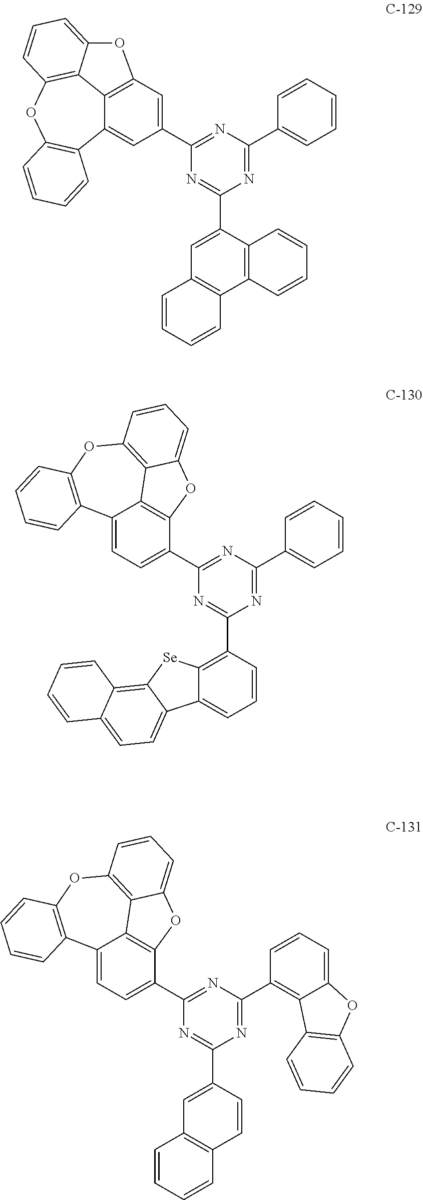 [SMILES: c1ccc(-c2nc(-c3cc4oc5cccc6oc7ccccc7c(c3)c4c65)nc(-c3cc4ccccc4c4ccccc34)n2)cc1 ; c1ccc(-c2nc(-c3ccc4c5ccccc5oc5cccc6oc3c4c56)nc(-c3cccc4c3[se]c3c5ccccc5ccc43)n2)cc1 ; c1ccc2cc(-c3nc(-c4ccc5c6ccccc6oc6cccc7oc4c5c67)nc(-c4cccc5oc6ccccc6c45)n3)ccc2c1]

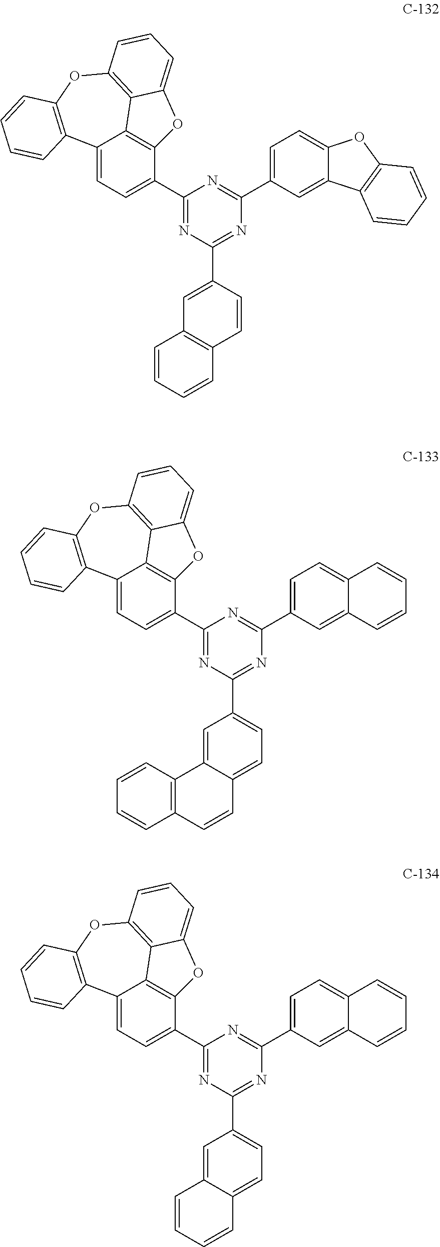 [SMILES: c1ccc2cc(-c3nc(-c4ccc5oc6ccccc6c5c4)nc(-c4ccc5c6ccccc6oc6cccc7oc4c5c67)n3)ccc2c1 ; c1ccc2cc(-c3nc(-c4ccc5ccc6ccccc6c5c4)nc(-c4ccc5c6ccccc6oc6cccc7oc4c5c67)n3)ccc2c1 ; c1ccc2cc(-c3nc(-c4ccc5ccccc5c4)nc(-c4ccc5c6ccccc6oc6cccc7oc4c5c67)n3)ccc2c1]

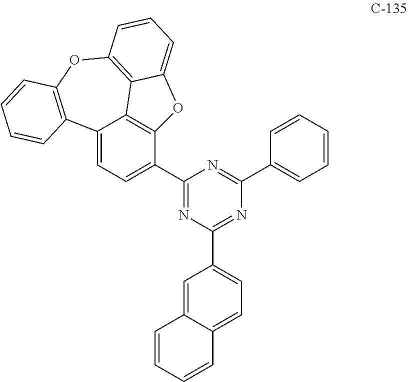 [SMILES: c1ccc(-c2nc(-c3ccc4ccccc4c3)nc(-c3ccc4c5ccccc5oc5cccc6oc3c4c56)n2)cc1]

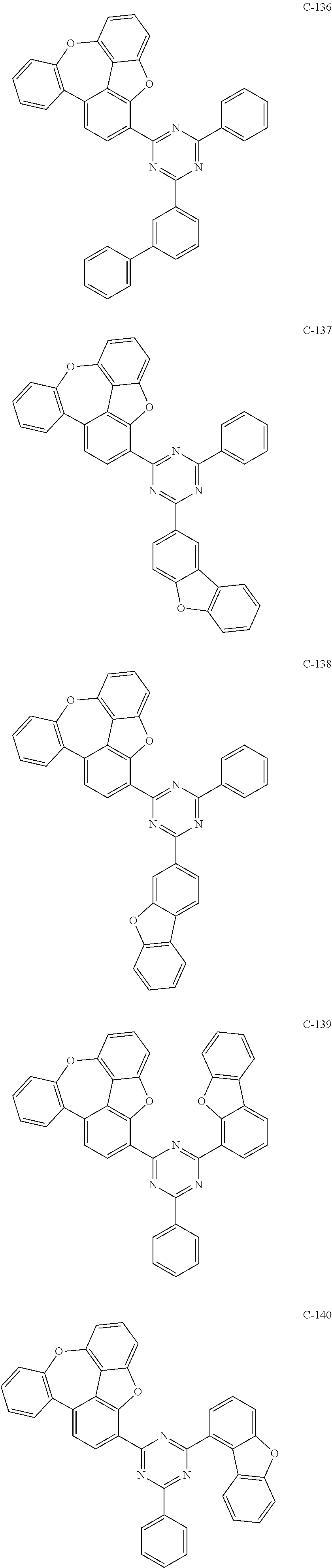 [SMILES: c1ccc(-c2cccc(-c3nc(-c4ccccc4)nc(-c4ccc5c6ccccc6oc6cccc7oc4c5c67)n3)c2)cc1 ; c1ccc(-c2nc(-c3ccc4oc5ccccc5c4c3)nc(-c3ccc4c5ccccc5oc5cccc6oc3c4c56)n2)cc1 ; c1ccc(-c2nc(-c3ccc4c(c3)oc3ccccc34)nc(-c3ccc4c5ccccc5oc5cccc6oc3c4c56)n2)cc1 ; c1ccc(-c2nc(-c3cccc4c3oc3ccccc34)nc(-c3ccc4c5ccccc5oc5cccc6oc3c4c56)n2)cc1 ; c1ccc(-c2nc(-c3ccc4c5ccccc5oc5cccc6oc3c4c56)nc(-c3cccc4oc5ccccc5c34)n2)cc1]

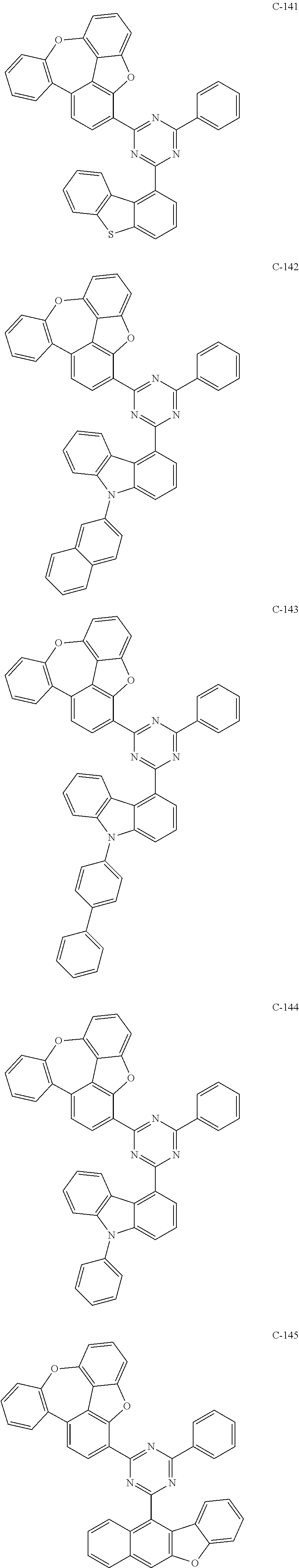

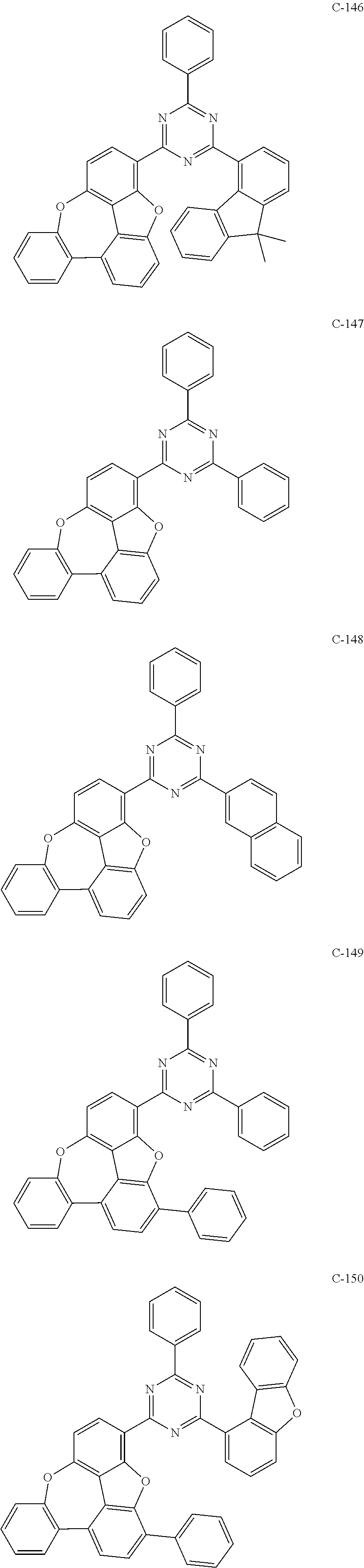 [SMILES: CC1(C)c2ccccc2-c2c(-c3nc(-c4ccccc4)nc(-c4ccc5oc6ccccc6c6cccc7oc4c5c76)n3)cccc21 ; c1ccc(-c2nc(-c3ccccc3)nc(-c3ccc4oc5ccccc5c5cccc6oc3c4c65)n2)cc1 ; c1ccc(-c2nc(-c3ccc4ccccc4c3)nc(-c3ccc4oc5ccccc5c5cccc6oc3c4c65)n2)cc1 ; c1ccc(-c2nc(-c3ccccc3)nc(-c3ccc4oc5ccccc5c5ccc(-c6ccccc6)c6oc3c4c65)n2)cc1 ; c1ccc(-c2nc(-c3ccc4oc5ccccc5c5ccc(-c6ccccc6)c6oc3c4c65)nc(-c3cccc4oc5ccccc5c34)n2)cc1]

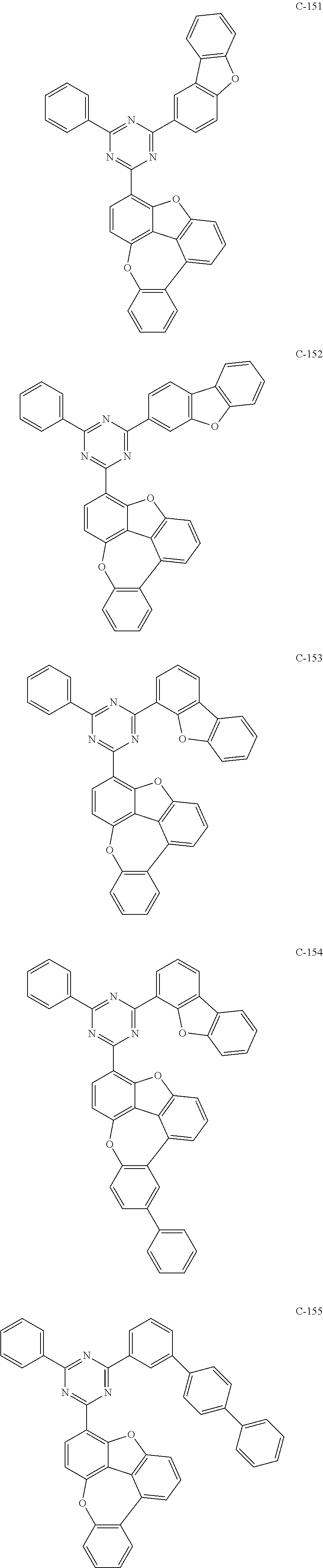 [SMILES: c1ccc(-c2nc(-c3ccc4oc5ccccc5c4c3)nc(-c3ccc4oc5ccccc5c5cccc6oc3c4c65)n2)cc1 ; c1ccc(-c2nc(-c3ccc4c(c3)oc3ccccc34)nc(-c3ccc4oc5ccccc5c5cccc6oc3c4c65)n2)cc1 ; c1ccc(-c2nc(-c3cccc4c3oc3ccccc34)nc(-c3ccc4oc5ccccc5c5cccc6oc3c4c65)n2)cc1 ; c1ccc(-c2ccc3oc4ccc(-c5nc(-c6ccccc6)nc(-c6cccc7c6oc6ccccc67)n5)c5oc6cccc(c3c2)c6c45)cc1 ; c1ccc(-c2ccc(-c3cccc(-c4nc(-c5ccccc5)nc(-c5ccc6oc7ccccc7c7cccc8oc5c6c87)n4)c3)cc2)cc1]

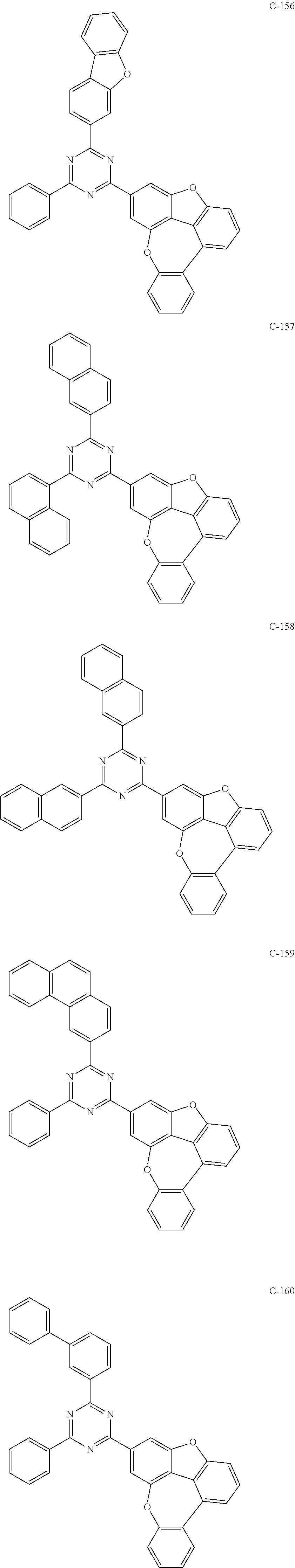 [SMILES: c1ccc(-c2nc(-c3ccc4c(c3)oc3ccccc34)nc(-c3cc4oc5ccccc5c5cccc6oc(c3)c4c65)n2)cc1 ; c1ccc2cc(-c3nc(-c4cc5oc6ccccc6c6cccc7oc(c4)c5c76)nc(-c4cccc5ccccc45)n3)ccc2c1 ; c1ccc2cc(-c3nc(-c4ccc5ccccc5c4)nc(-c4cc5oc6ccccc6c6cccc7oc(c4)c5c76)n3)ccc2c1 ; c1ccc(-c2nc(-c3cc4oc5ccccc5c5cccc6oc(c3)c4c65)nc(-c3ccc4ccc5ccccc5c4c3)n2)cc1 ; c1ccc(-c2cccc(-c3nc(-c4ccccc4)nc(-c4cc5oc6ccccc6c6cccc7oc(c4)c5c76)n3)c2)cc1]

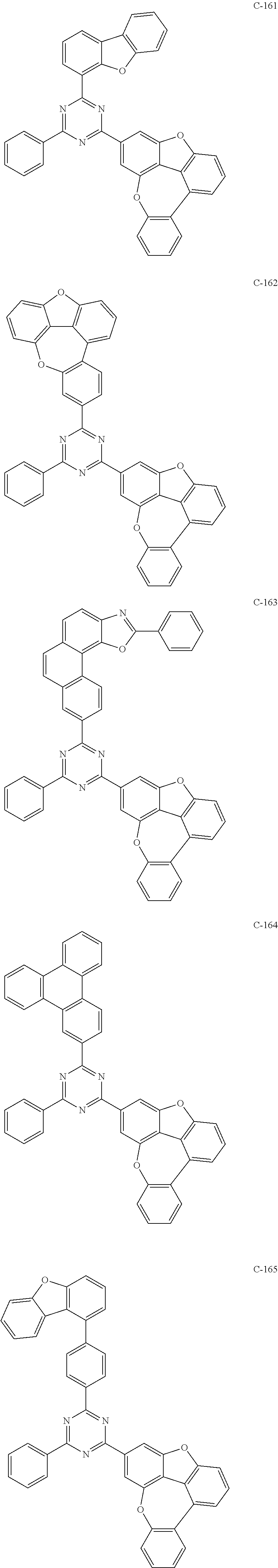

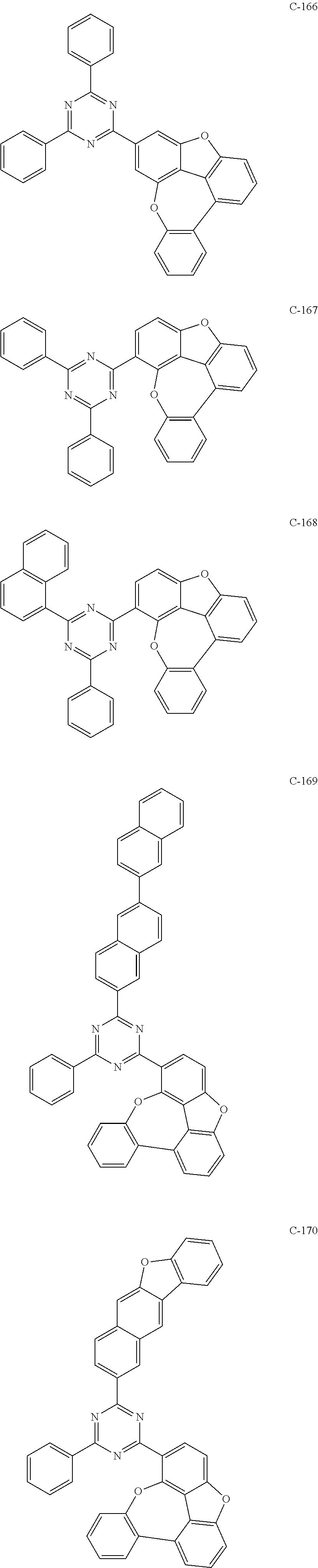 [SMILES: c1ccc(-c2nc(-c3ccccc3)nc(-c3cc4oc5ccccc5c5cccc6oc(c3)c4c65)n2)cc1 ; c1ccc(-c2nc(-c3ccccc3)nc(-c3ccc4oc5cccc6c7ccccc7oc3c4c56)n2)cc1 ; c1ccc(-c2nc(-c3cccc4ccccc34)nc(-c3ccc4oc5cccc6c7ccccc7oc3c4c56)n2)cc1 ; c1ccc(-c2nc(-c3ccc4cc(-c5ccc6ccccc6c5)ccc4c3)nc(-c3ccc4oc5cccc6c7ccccc7oc3c4c56)n2)cc1 ; c1ccc(-c2nc(-c3ccc4cc5oc6ccccc6c5cc4c3)nc(-c3ccc4oc5cccc6c7ccccc7oc3c4c56)n2)cc1]

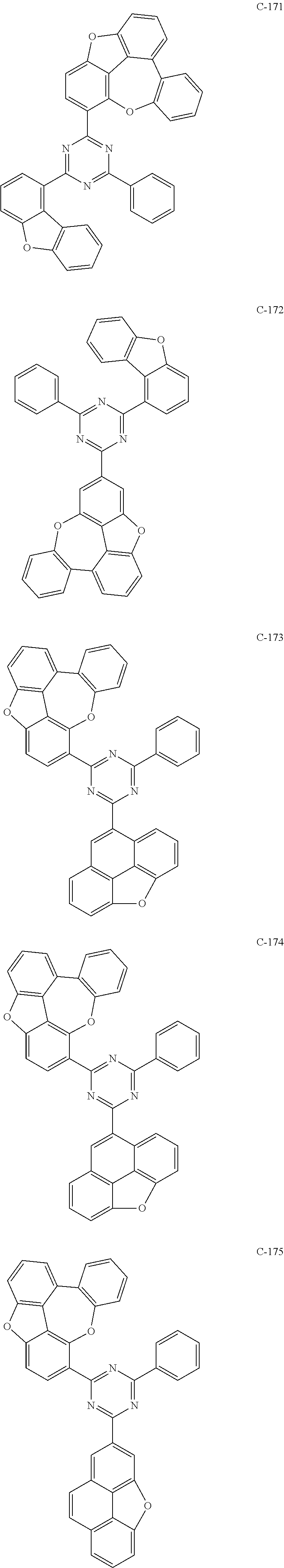 [SMILES: c1ccc(-c2nc(-c3ccc4oc5cccc6c7ccccc7oc3c4c56)nc(-c3cccc4oc5ccccc5c34)n2)cc1 ; c1ccc(-c2nc(-c3cc4oc5ccccc5c5cccc6oc(c3)c4c65)nc(-c3cccc4oc5ccccc5c34)n2)cc1 ; c1ccc(-c2nc(-c3cc4cccc5oc6cccc3c6c45)nc(-c3ccc4oc5cccc6c7ccccc7oc3c4c56)n2)cc1 ; c1ccc(-c2nc(-c3cc4cccc5oc6cccc3c6c45)nc(-c3ccc4oc5cccc6c7ccccc7oc3c4c56)n2)cc1 ; c1ccc(-c2nc(-c3cc4ccc5cccc6oc(c3)c4c56)nc(-c3ccc4oc5cccc6c7ccccc7oc3c4c56)n2)cc1]

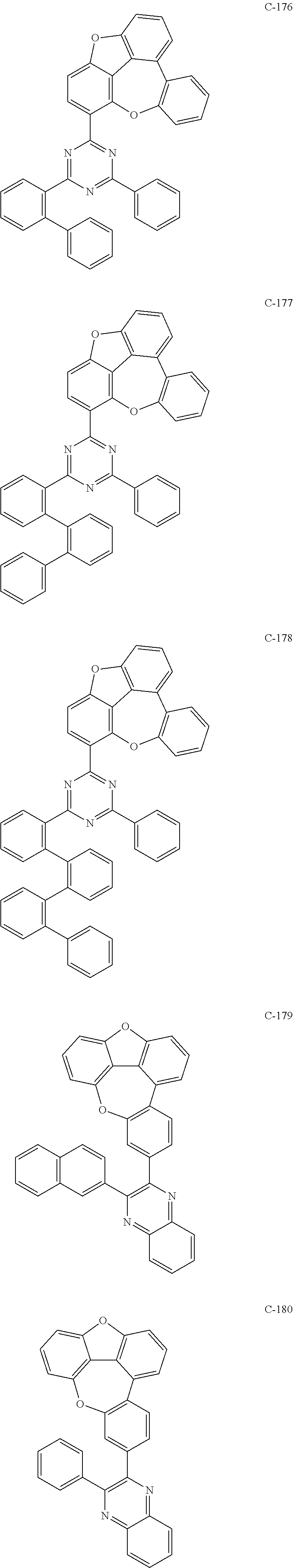 [SMILES: c1ccc(-c2nc(-c3ccccc3-c3ccccc3)nc(-c3ccc4oc5cccc6c7ccccc7oc3c4c56)n2)cc1 ; c1ccc(-c2nc(-c3ccccc3-c3ccccc3-c3ccccc3)nc(-c3ccc4oc5cccc6c7ccccc7oc3c4c56)n2)cc1 ; c1ccc(-c2nc(-c3ccccc3-c3ccccc3-c3ccccc3-c3ccccc3)nc(-c3ccc4oc5cccc6c7ccccc7oc3c4c56)n2)cc1 ; c1ccc2cc(-c3nc4ccccc4nc3-c3ccc4c(c3)oc3cccc5oc6cccc4c6c35)ccc2c1 ; c1ccc(-c2nc3ccccc3nc2-c2ccc3c(c2)oc2cccc4oc5cccc3c5c24)cc1]

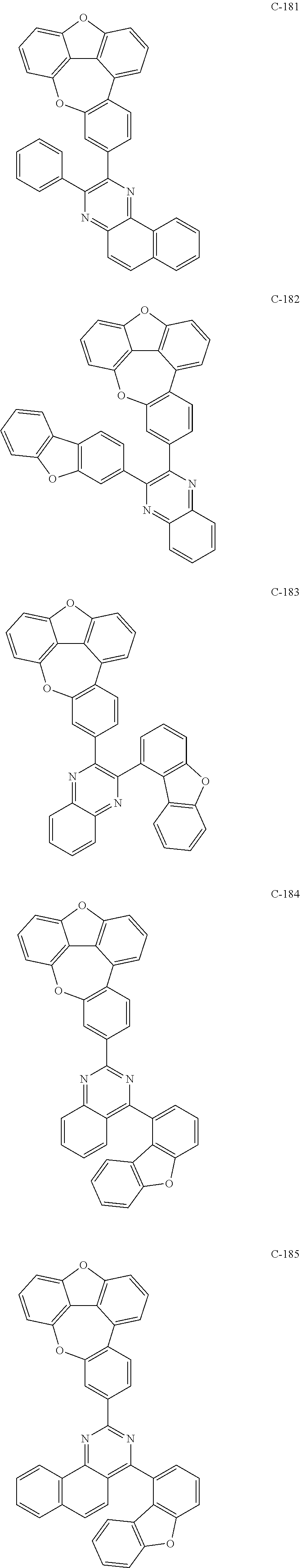 [SMILES: c1ccc(-c2nc3ccc4ccccc4c3nc2-c2ccc3c(c2)oc2cccc4oc5cccc3c5c24)cc1 ; c1ccc2nc(-c3ccc4c(c3)oc3cccc5oc6cccc4c6c35)c(-c3ccc4c(c3)oc3ccccc34)nc2c1 ; c1ccc2nc(-c3cccc4oc5ccccc5c34)c(-c3ccc4c(c3)oc3cccc5oc6cccc4c6c35)nc2c1 ; c1ccc2c(-c3cccc4oc5ccccc5c34)nc(-c3ccc4c(c3)oc3cccc5oc6cccc4c6c35)nc2c1 ; c1ccc2c(c1)ccc1c(-c3cccc4oc5ccccc5c34)nc(-c3ccc4c(c3)oc3cccc5oc6cccc4c6c35)nc12]

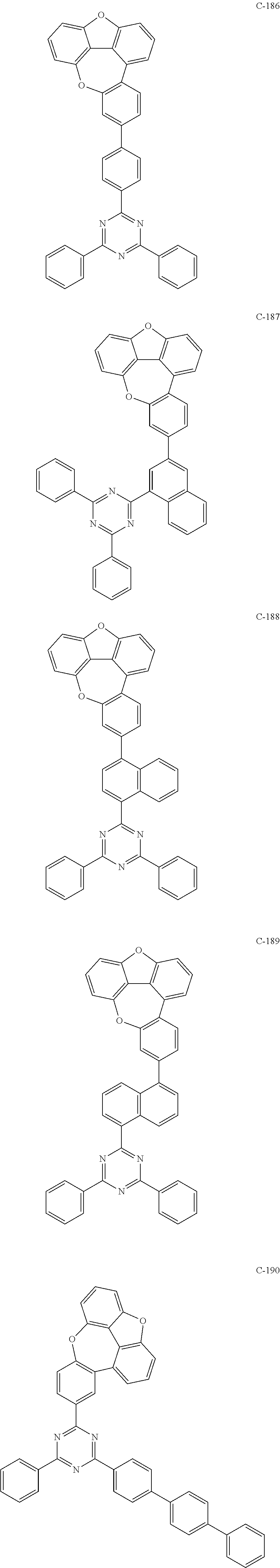

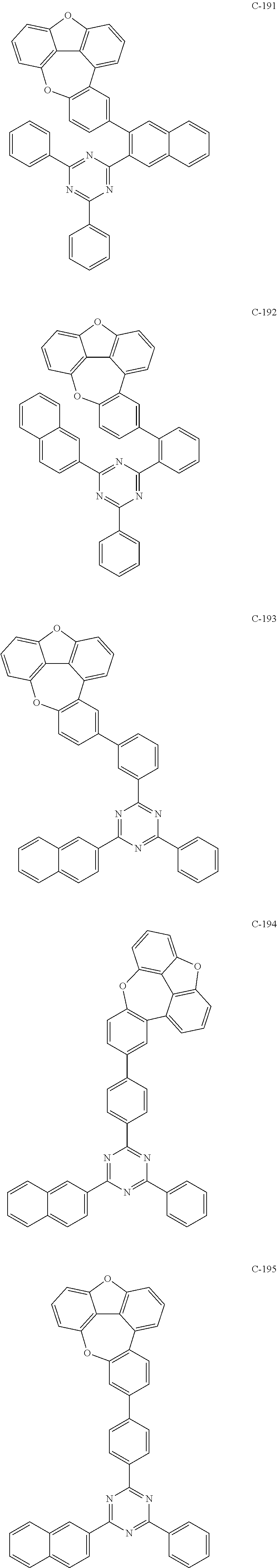 [SMILES: c1ccc(-c2nc(-c3ccccc3)nc(-c3cc4ccccc4cc3-c3ccc4oc5cccc6oc7cccc(c4c3)c7c56)n2)cc1 ; c1ccc(-c2nc(-c3ccc4ccccc4c3)nc(-c3ccccc3-c3ccc4oc5cccc6oc7cccc(c4c3)c7c56)n2)cc1 ; c1ccc(-c2nc(-c3cccc(-c4ccc5oc6cccc7oc8cccc(c5c4)c8c67)c3)nc(-c3ccc4ccccc4c3)n2)cc1 ; c1ccc(-c2nc(-c3ccc(-c4ccc5oc6cccc7oc8cccc(c5c4)c8c67)cc3)nc(-c3ccc4ccccc4c3)n2)cc1 ; c1ccc(-c2nc(-c3ccc(-c4ccc5c(c4)oc4cccc6oc7cccc5c7c46)cc3)nc(-c3ccc4ccccc4c3)n2)cc1]

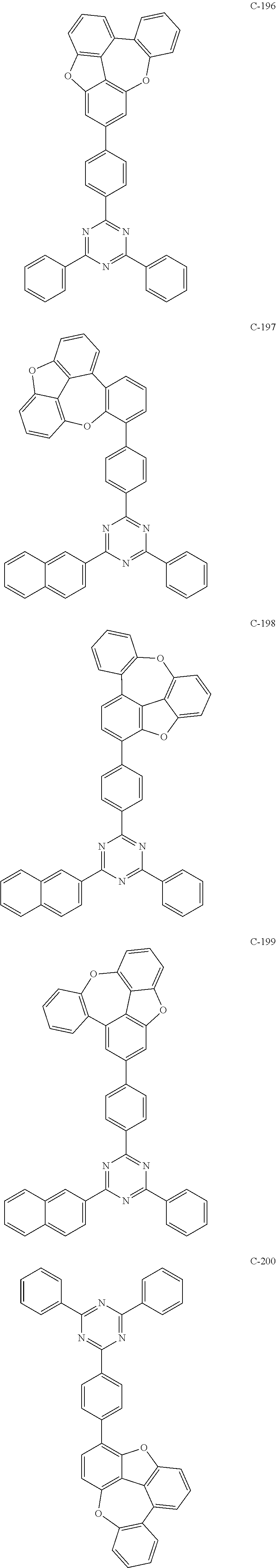 [SMILES: c1ccc(-c2nc(-c3ccccc3)nc(-c3ccc(-c4cc5oc6ccccc6c6cccc7oc(c4)c5c76)cc3)n2)cc1 ; c1ccc(-c2nc(-c3ccc(-c4cccc5c4oc4cccc6oc7cccc5c7c64)cc3)nc(-c3ccc4ccccc4c3)n2)cc1 ; c1ccc(-c2nc(-c3ccc(-c4ccc5c6ccccc6oc6cccc7oc4c5c67)cc3)nc(-c3ccc4ccccc4c3)n2)cc1 ; c1ccc(-c2nc(-c3ccc(-c4cc5oc6cccc7oc8ccccc8c(c4)c5c76)cc3)nc(-c3ccc4ccccc4c3)n2)cc1 ; c1ccc(-c2nc(-c3ccccc3)nc(-c3ccc(-c4ccc5oc6ccccc6c6cccc7oc4c5c76)cc3)n2)cc1]

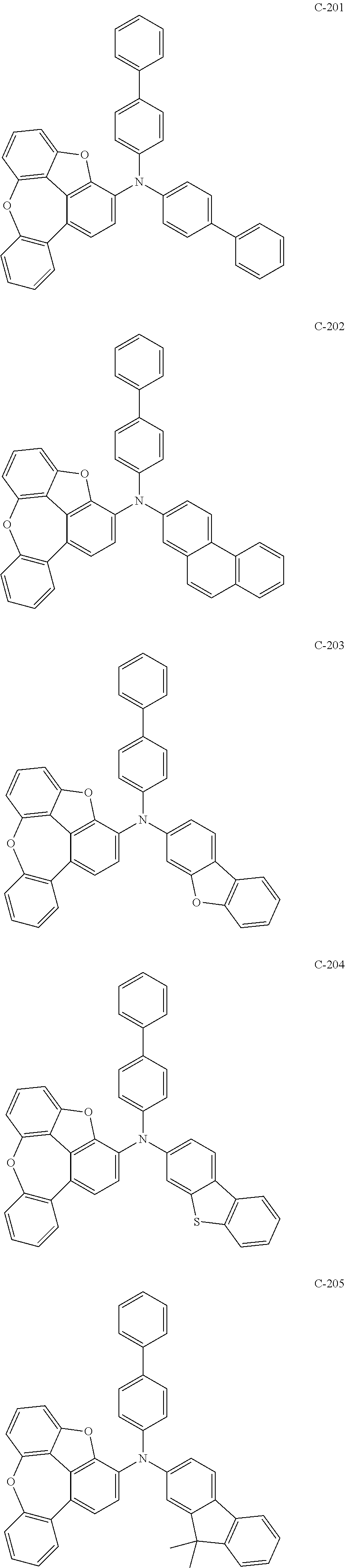 [SMILES: c1ccc(-c2ccc(N(c3ccc(-c4ccccc4)cc3)c3ccc4c5ccccc5oc5cccc6oc3c4c56)cc2)cc1 ; c1ccc(-c2ccc(N(c3ccc4c(ccc5ccccc54)c3)c3ccc4c5ccccc5oc5cccc6oc3c4c56)cc2)cc1 ; c1ccc(-c2ccc(N(c3ccc4c(c3)oc3ccccc34)c3ccc4c5ccccc5oc5cccc6oc3c4c56)cc2)cc1 ; c1ccc(-c2ccc(N(c3ccc4c(c3)sc3ccccc34)c3ccc4c5ccccc5oc5cccc6oc3c4c56)cc2)cc1 ; CC1(C)c2ccccc2-c2ccc(N(c3ccc(-c4ccccc4)cc3)c3ccc4c5ccccc5oc5cccc6oc3c4c56)cc21]

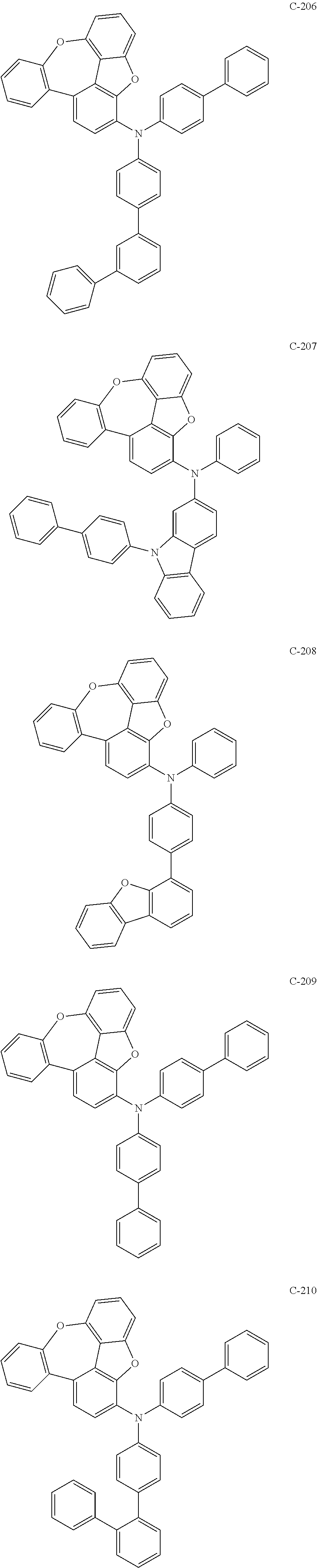 [SMILES: c1ccc(-c2ccc(N(c3ccc(-c4cccc(-c5ccccc5)c4)cc3)c3ccc4c5ccccc5oc5cccc6oc3c4c56)cc2)cc1 ; c1ccc(-c2ccc(-n3c4ccccc4c4ccc(N(c5ccccc5)c5ccc6c7ccccc7oc7cccc8oc5c6c78)cc43)cc2)cc1 ; c1ccc(N(c2ccc(-c3cccc4c3oc3ccccc34)cc2)c2ccc3c4ccccc4oc4cccc5oc2c3c45)cc1 ; c1ccc(-c2ccc(N(c3ccc(-c4ccccc4)cc3)c3ccc4c5ccccc5oc5cccc6oc3c4c56)cc2)cc1 ; c1ccc(-c2ccc(N(c3ccc(-c4ccccc4-c4ccccc4)cc3)c3ccc4c5ccccc5oc5cccc6oc3c4c56)cc2)cc1]

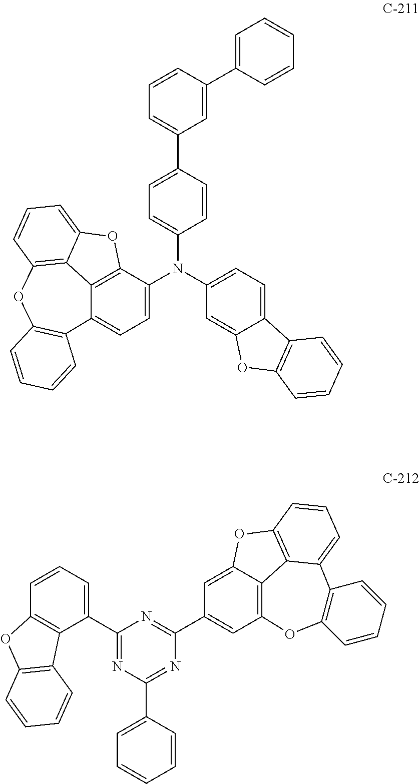 [SMILES: c1ccc(-c2cccc(-c3ccc(N(c4ccc5c(c4)oc4ccccc45)c4ccc5c6ccccc6oc6cccc7oc4c5c67)cc3)c2)cc1 ; c1ccc(-c2nc(-c3cc4oc5ccccc5c5cccc6oc(c3)c4c65)nc(-c3cccc4oc5ccccc5c34)n2)cc1]

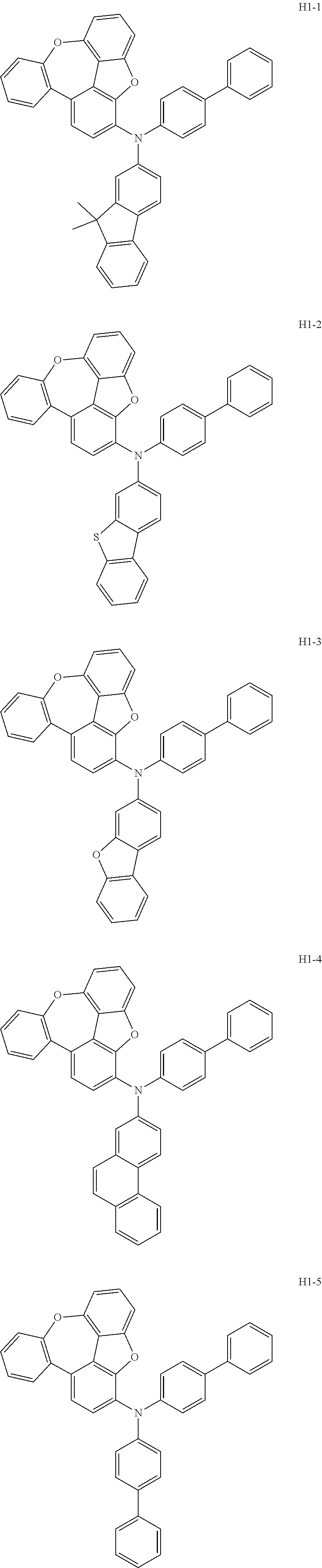 [SMILES: CC1(C)c2ccccc2-c2ccc(N(c3ccc(-c4ccccc4)cc3)c3ccc4c5ccccc5oc5cccc6oc3c4c56)cc21 ; c1ccc(-c2ccc(N(c3ccc4c(c3)sc3ccccc34)c3ccc4c5ccccc5oc5cccc6oc3c4c56)cc2)cc1 ; c1ccc(-c2ccc(N(c3ccc4c(c3)oc3ccccc34)c3ccc4c5ccccc5oc5cccc6oc3c4c56)cc2)cc1 ; c1ccc(-c2ccc(N(c3ccc4c(ccc5ccccc54)c3)c3ccc4c5ccccc5oc5cccc6oc3c4c56)cc2)cc1 ; c1ccc(-c2ccc(N(c3ccc(-c4ccccc4)cc3)c3ccc4c5ccccc5oc5cccc6oc3c4c56)cc2)cc1]

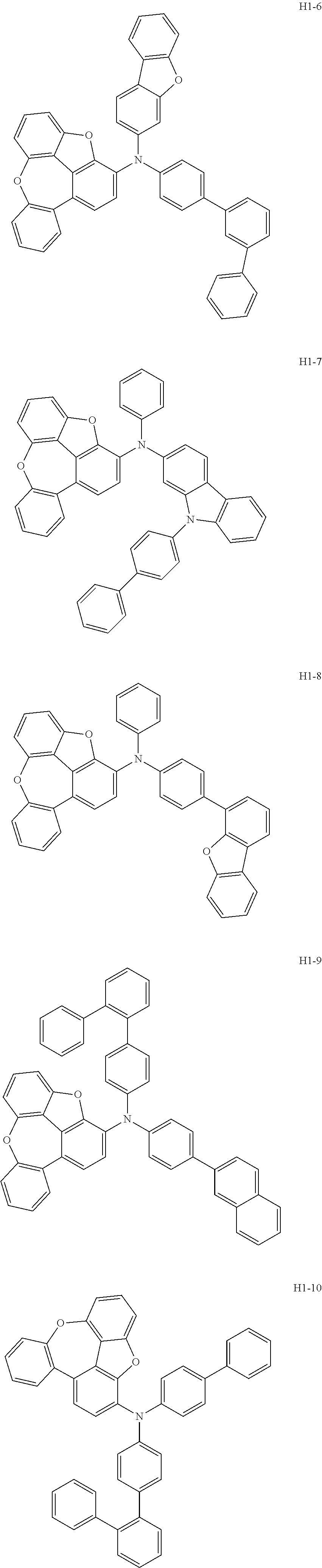 [SMILES: c1ccc(-c2cccc(-c3ccc(N(c4ccc5c(c4)oc4ccccc45)c4ccc5c6ccccc6oc6cccc7oc4c5c67)cc3)c2)cc1 ; c1ccc(-c2ccc(-n3c4ccccc4c4ccc(N(c5ccccc5)c5ccc6c7ccccc7oc7cccc8oc5c6c78)cc43)cc2)cc1 ; c1ccc(N(c2ccc(-c3cccc4c3oc3ccccc34)cc2)c2ccc3c4ccccc4oc4cccc5oc2c3c45)cc1 ; c1ccc(-c2ccccc2-c2ccc(N(c3ccc(-c4ccc5ccccc5c4)cc3)c3ccc4c5ccccc5oc5cccc6oc3c4c56)cc2)cc1 ; c1ccc(-c2ccc(N(c3ccc(-c4ccccc4-c4ccccc4)cc3)c3ccc4c5ccccc5oc5cccc6oc3c4c56)cc2)cc1]

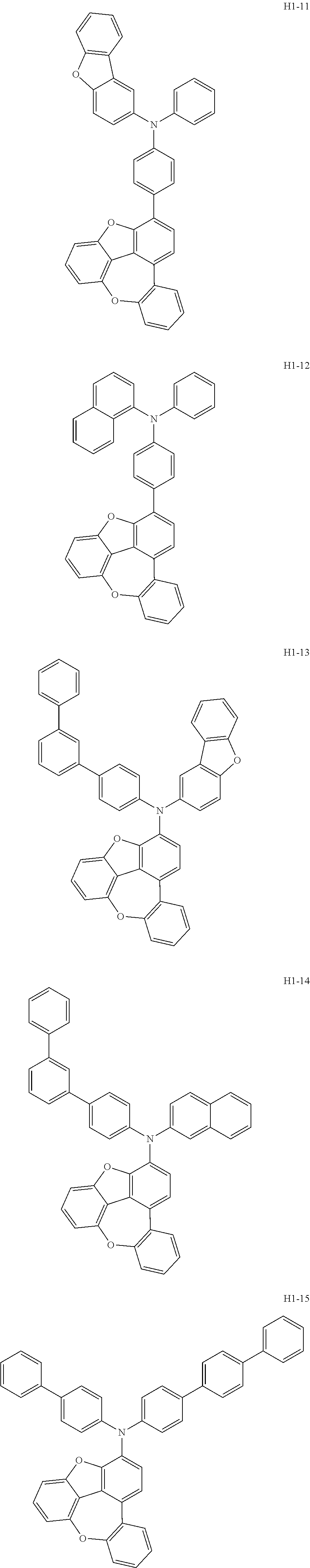 [SMILES: c1ccc(N(c2ccc(-c3ccc4c5ccccc5oc5cccc6oc3c4c56)cc2)c2ccc3oc4ccccc4c3c2)cc1 ; c1ccc(N(c2ccc(-c3ccc4c5ccccc5oc5cccc6oc3c4c56)cc2)c2cccc3ccccc23)cc1 ; c1ccc(-c2cccc(-c3ccc(N(c4ccc5oc6ccccc6c5c4)c4ccc5c6ccccc6oc6cccc7oc4c5c67)cc3)c2)cc1 ; c1ccc(-c2cccc(-c3ccc(N(c4ccc5ccccc5c4)c4ccc5c6ccccc6oc6cccc7oc4c5c67)cc3)c2)cc1 ; c1ccc(-c2ccc(-c3ccc(N(c4ccc(-c5ccccc5)cc4)c4ccc5c6ccccc6oc6cccc7oc4c5c67)cc3)cc2)cc1]

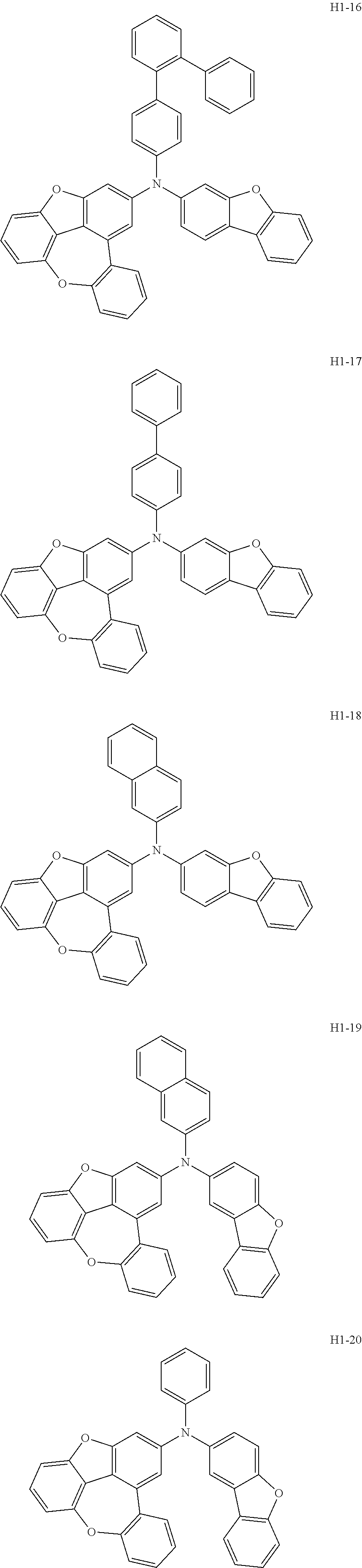 [SMILES: c1ccc(-c2ccccc2-c2ccc(N(c3ccc4c(c3)oc3ccccc34)c3cc4oc5cccc6oc7ccccc7c(c3)c4c65)cc2)cc1 ; c1ccc(-c2ccc(N(c3ccc4c(c3)oc3ccccc34)c3cc4oc5cccc6oc7ccccc7c(c3)c4c65)cc2)cc1 ; c1ccc2cc(N(c3ccc4c(c3)oc3ccccc34)c3cc4oc5cccc6oc7ccccc7c(c3)c4c65)ccc2c1 ; c1ccc2cc(N(c3ccc4oc5ccccc5c4c3)c3cc4oc5cccc6oc7ccccc7c(c3)c4c65)ccc2c1 ; c1ccc(N(c2ccc3oc4ccccc4c3c2)c2cc3oc4cccc5oc6ccccc6c(c2)c3c54)cc1]

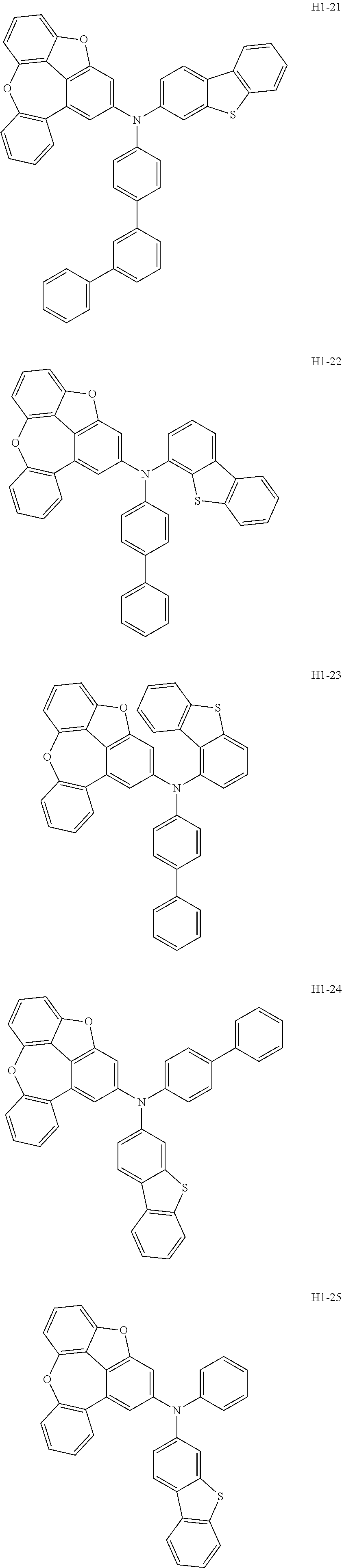 [SMILES: c1ccc(-c2cccc(-c3ccc(N(c4ccc5c(c4)sc4ccccc45)c4cc5oc6cccc7oc8ccccc8c(c4)c5c76)cc3)c2)cc1 ; c1ccc(-c2ccc(N(c3cc4oc5cccc6oc7ccccc7c(c3)c4c65)c3cccc4c3sc3ccccc34)cc2)cc1 ; c1ccc(-c2ccc(N(c3cc4oc5cccc6oc7ccccc7c(c3)c4c65)c3cccc4sc5ccccc5c34)cc2)cc1 ; c1ccc(-c2ccc(N(c3ccc4c(c3)sc3ccccc34)c3cc4oc5cccc6oc7ccccc7c(c3)c4c65)cc2)cc1 ; c1ccc(N(c2ccc3c(c2)sc2ccccc23)c2cc3oc4cccc5oc6ccccc6c(c2)c3c54)cc1]

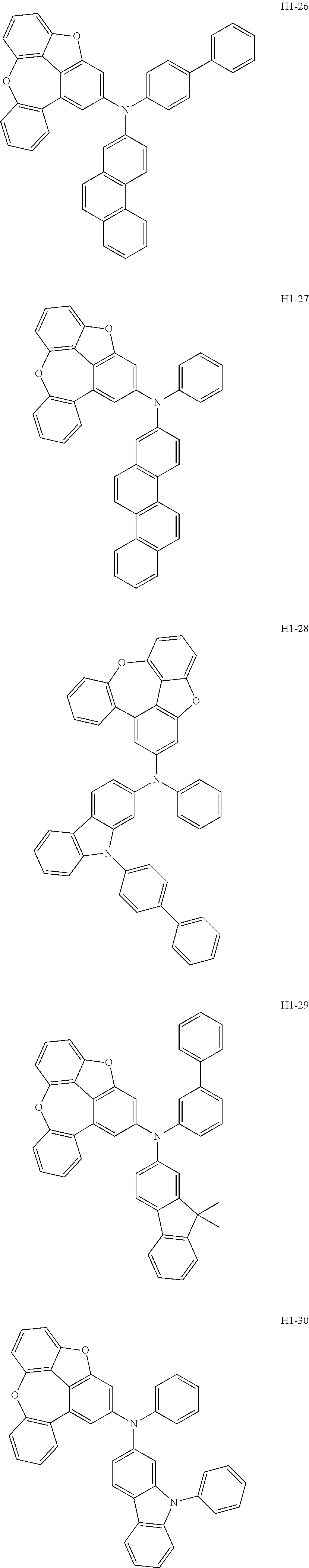 [SMILES: c1ccc(-c2ccc(N(c3ccc4c(ccc5ccccc54)c3)c3cc4oc5cccc6oc7ccccc7c(c3)c4c65)cc2)cc1 ; c1ccc(N(c2ccc3c(ccc4c5ccccc5ccc34)c2)c2cc3oc4cccc5oc6ccccc6c(c2)c3c54)cc1 ; c1ccc(-c2ccc(-n3c4ccccc4c4ccc(N(c5ccccc5)c5cc6oc7cccc8oc9ccccc9c(c5)c6c87)cc43)cc2)cc1 ; CC1(C)c2ccccc2-c2ccc(N(c3cccc(-c4ccccc4)c3)c3cc4oc5cccc6oc7ccccc7c(c3)c4c65)cc21 ; c1ccc(N(c2cc3oc4cccc5oc6ccccc6c(c2)c3c54)c2ccc3c4ccccc4n(-c4ccccc4)c3c2)cc1]

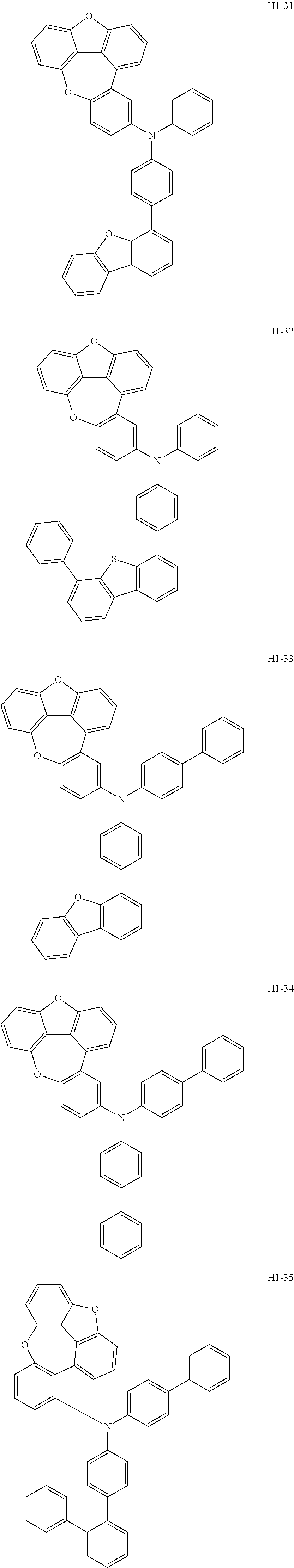 [SMILES: c1ccc(N(c2ccc(-c3cccc4c3oc3ccccc34)cc2)c2ccc3oc4cccc5oc6cccc(c3c2)c6c45)cc1 ; c1ccc(-c2cccc3c2sc2c(-c4ccc(N(c5ccccc5)c5ccc6oc7cccc8oc9cccc(c6c5)c9c78)cc4)cccc23)cc1 ; c1ccc(-c2ccc(N(c3ccc(-c4cccc5c4oc4ccccc45)cc3)c3ccc4oc5cccc6oc7cccc(c4c3)c7c56)cc2)cc1 ; c1ccc(-c2ccc(N(c3ccc(-c4ccccc4)cc3)c3ccc4oc5cccc6oc7cccc(c4c3)c7c56)cc2)cc1 ; c1ccc(-c2ccc(N(c3ccc(-c4ccccc4-c4ccccc4)cc3)c3cccc4oc5cccc6oc7cccc(c34)c7c56)cc2)cc1]

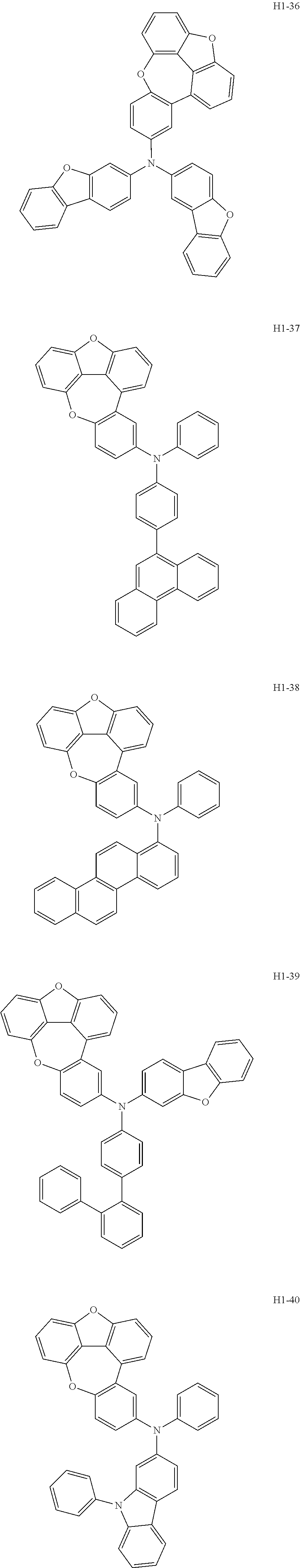 [SMILES: c1ccc2c(c1)oc1cc(N(c3ccc4oc5ccccc5c4c3)c3ccc4oc5cccc6oc7cccc(c4c3)c7c56)ccc12 ; c1ccc(N(c2ccc(-c3cc4ccccc4c4ccccc34)cc2)c2ccc3oc4cccc5oc6cccc(c3c2)c6c45)cc1 ; c1ccc(N(c2ccc3oc4cccc5oc6cccc(c3c2)c6c45)c2cccc3c2ccc2c4ccccc4ccc32)cc1 ; c1ccc(-c2ccccc2-c2ccc(N(c3ccc4c(c3)oc3ccccc34)c3ccc4oc5cccc6oc7cccc(c4c3)c7c56)cc2)cc1 ; c1ccc(N(c2ccc3oc4cccc5oc6cccc(c3c2)c6c45)c2ccc3c4ccccc4n(-c4ccccc4)c3c2)cc1]

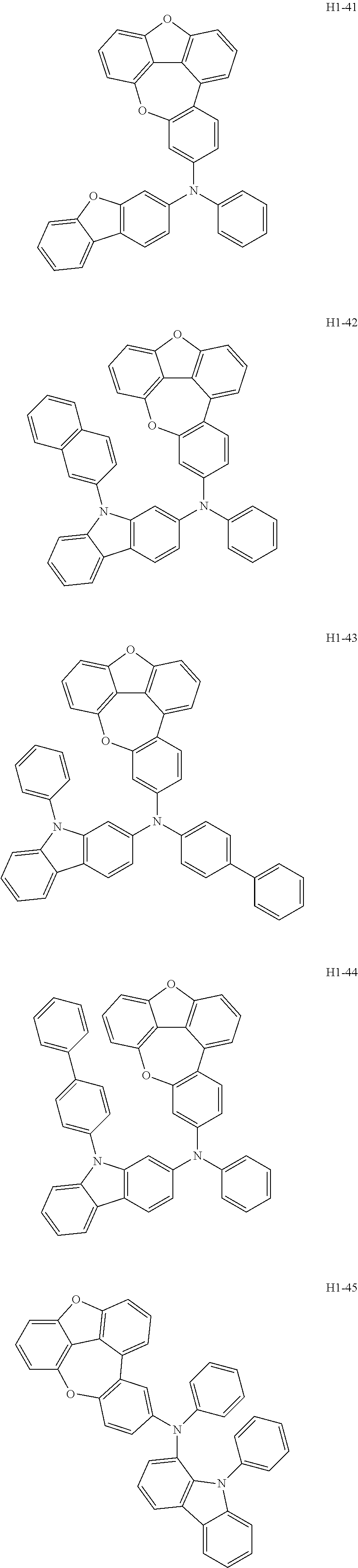 [SMILES: c1ccc(N(c2ccc3c(c2)oc2ccccc23)c2ccc3c(c2)oc2cccc4oc5cccc3c5c24)cc1 ; c1ccc(N(c2ccc3c(c2)oc2cccc4oc5cccc3c5c24)c2ccc3c4ccccc4n(-c4ccc5ccccc5c4)c3c2)cc1 ; c1ccc(-c2ccc(N(c3ccc4c(c3)oc3cccc5oc6cccc4c6c35)c3ccc4c5ccccc5n(-c5ccccc5)c4c3)cc2)cc1 ; c1ccc(-c2ccc(-n3c4ccccc4c4ccc(N(c5ccccc5)c5ccc6c(c5)oc5cccc7oc8cccc6c8c57)cc43)cc2)cc1 ; c1ccc(N(c2ccc3oc4cccc5oc6cccc(c3c2)c6c45)c2cccc3c4ccccc4n(-c4ccccc4)c23)cc1]

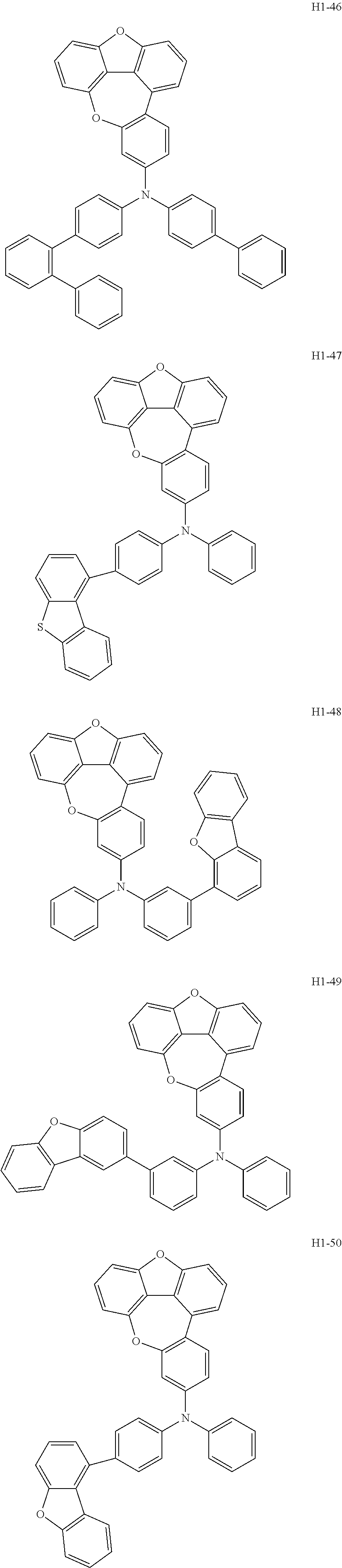 [SMILES: c1ccc(-c2ccc(N(c3ccc(-c4ccccc4-c4ccccc4)cc3)c3ccc4c(c3)oc3cccc5oc6cccc4c6c35)cc2)cc1 ; c1ccc(N(c2ccc(-c3cccc4sc5ccccc5c34)cc2)c2ccc3c(c2)oc2cccc4oc5cccc3c5c24)cc1 ; c1ccc(N(c2cccc(-c3cccc4c3oc3ccccc34)c2)c2ccc3c(c2)oc2cccc4oc5cccc3c5c24)cc1 ; c1ccc(N(c2cccc(-c3ccc4oc5ccccc5c4c3)c2)c2ccc3c(c2)oc2cccc4oc5cccc3c5c24)cc1 ; c1ccc(N(c2ccc(-c3cccc4oc5ccccc5c34)cc2)c2ccc3c(c2)oc2cccc4oc5cccc3c5c24)cc1]

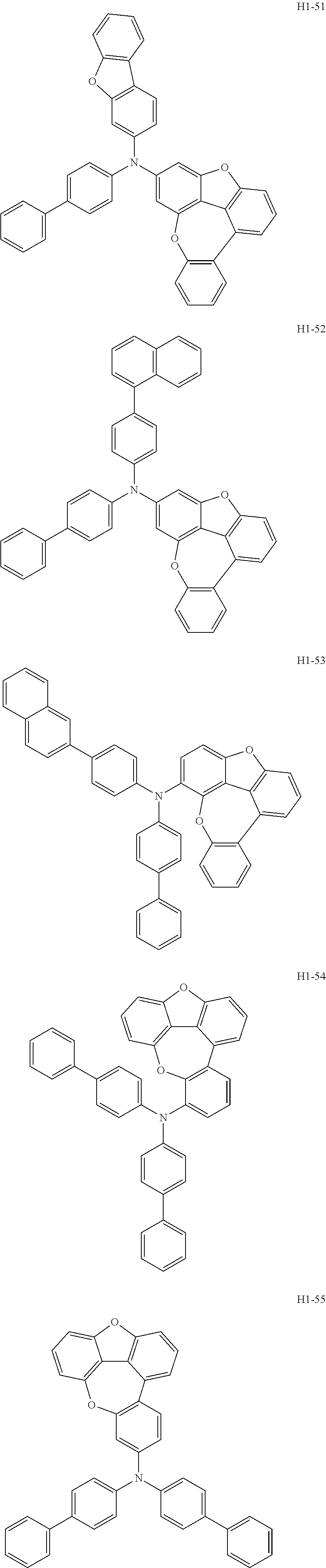

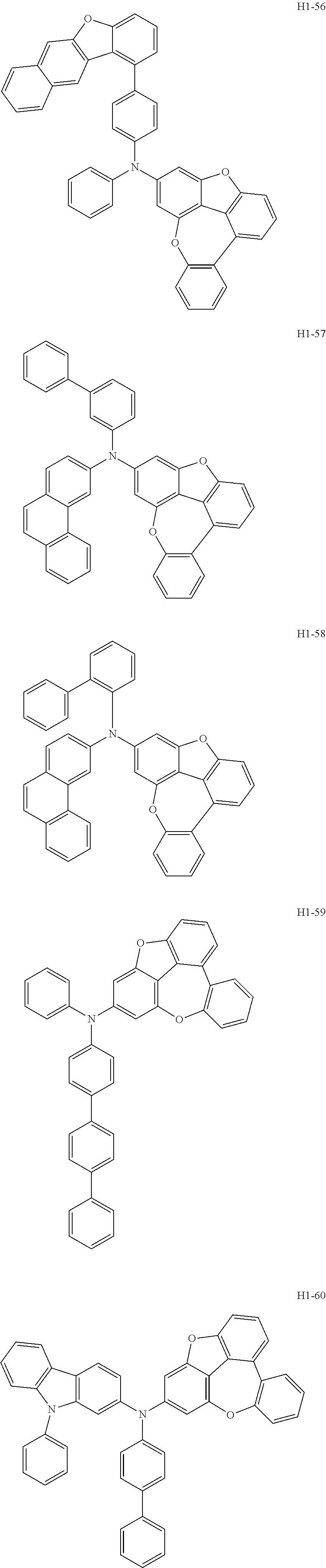 [SMILES: c1ccc(N(c2ccc(-c3cccc4oc5cc6ccccc6cc5c34)cc2)c2cc3oc4ccccc4c4cccc5oc(c2)c3c54)cc1 ; c1ccc(-c2cccc(N(c3cc4oc5ccccc5c5cccc6oc(c3)c4c65)c3ccc4ccc5ccccc5c4c3)c2)cc1 ; c1ccc(-c2ccccc2N(c2cc3oc4ccccc4c4cccc5oc(c2)c3c54)c2ccc3ccc4ccccc4c3c2)cc1 ; c1ccc(-c2ccc(-c3ccc(N(c4ccccc4)c4cc5oc6ccccc6c6cccc7oc(c4)c5c76)cc3)cc2)cc1 ; c1ccc(-c2ccc(N(c3cc4oc5ccccc5c5cccc6oc(c3)c4c65)c3ccc4c5ccccc5n(-c5ccccc5)c4c3)cc2)cc1]

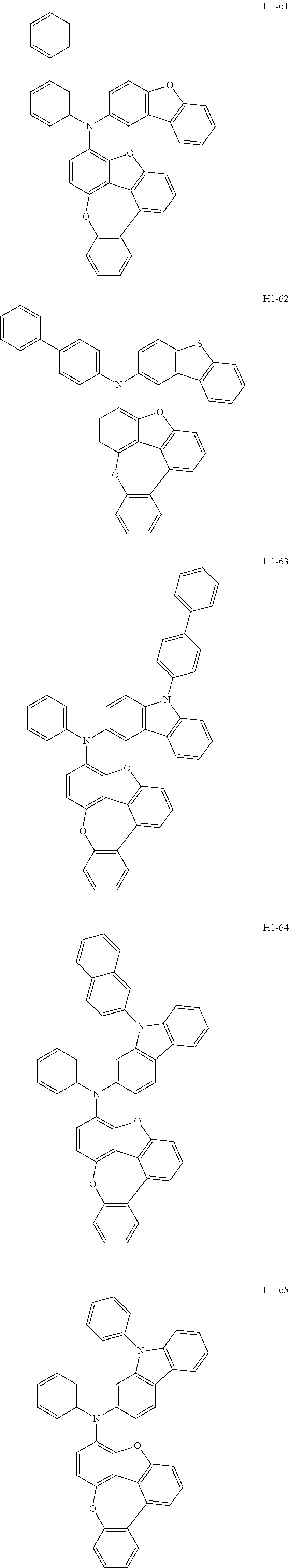 [SMILES: c1ccc(-c2cccc(N(c3ccc4oc5ccccc5c4c3)c3ccc4oc5ccccc5c5cccc6oc3c4c65)c2)cc1 ; c1ccc(-c2ccc(N(c3ccc4sc5ccccc5c4c3)c3ccc4oc5ccccc5c5cccc6oc3c4c65)cc2)cc1 ; c1ccc(-c2ccc(-n3c4ccccc4c4cc(N(c5ccccc5)c5ccc6oc7ccccc7c7cccc8oc5c6c87)ccc43)cc2)cc1 ; c1ccc(N(c2ccc3c4ccccc4n(-c4ccc5ccccc5c4)c3c2)c2ccc3oc4ccccc4c4cccc5oc2c3c54)cc1 ; c1ccc(N(c2ccc3c4ccccc4n(-c4ccccc4)c3c2)c2ccc3oc4ccccc4c4cccc5oc2c3c54)cc1]

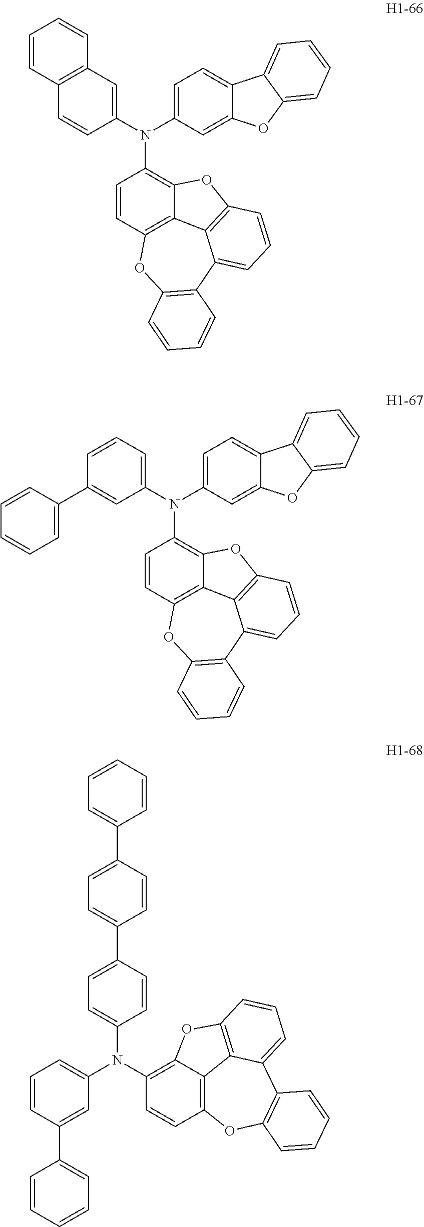 [SMILES: c1ccc2cc(N(c3ccc4c(c3)oc3ccccc34)c3ccc4oc5ccccc5c5cccc6oc3c4c65)ccc2c1 ; c1ccc(-c2cccc(N(c3ccc4c(c3)oc3ccccc34)c3ccc4oc5ccccc5c5cccc6oc3c4c65)c2)cc1 ; c1ccc(-c2ccc(-c3ccc(N(c4cccc(-c5ccccc5)c4)c4ccc5oc6ccccc6c6cccc7oc4c5c76)cc3)cc2)cc1]

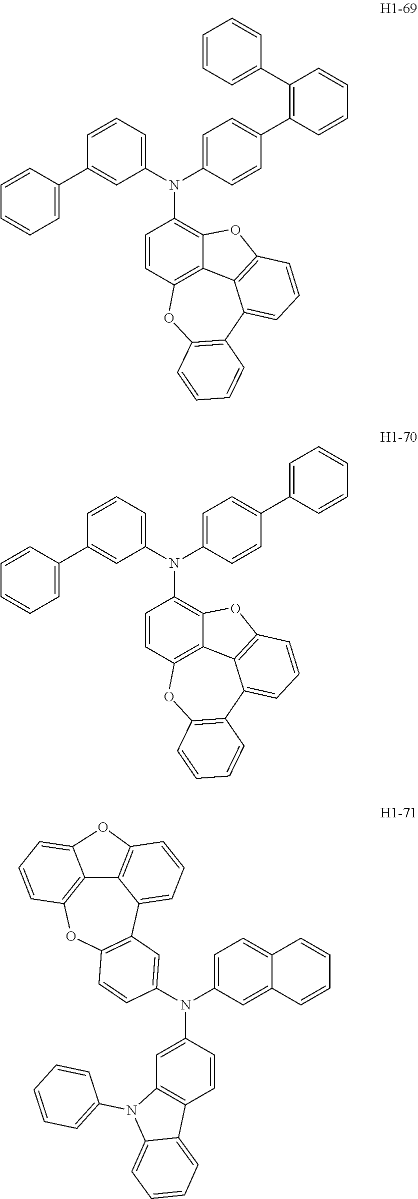 [SMILES: c1ccc(-c2cccc(N(c3ccc(-c4ccccc4-c4ccccc4)cc3)c3ccc4oc5ccccc5c5cccc6oc3c4c65)c2)cc1 ; c1ccc(-c2ccc(N(c3cccc(-c4ccccc4)c3)c3ccc4oc5ccccc5c5cccc6oc3c4c65)cc2)cc1 ; c1ccc(-n2c3ccccc3c3ccc(N(c4ccc5ccccc5c4)c4ccc5oc6cccc7oc8cccc(c5c4)c8c67)cc32)cc1]

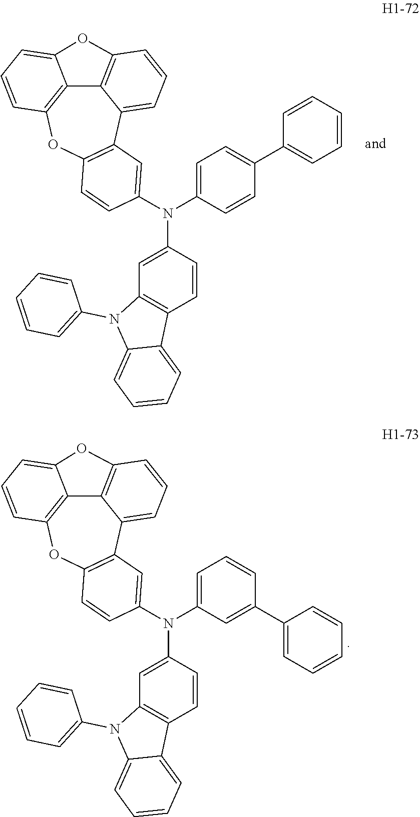 [SMILES: c1ccc(-c2ccc(N(c3ccc4oc5cccc6oc7cccc(c4c3)c7c56)c3ccc4c5ccccc5n(-c5ccccc5)c4c3)cc2)cc1 ; c1ccc(-c2cccc(N(c3ccc4oc5cccc6oc7cccc(c4c3)c7c56)c3ccc4c5ccccc5n(-c5ccccc5)c4c3)c2)cc1]

7. An organic electroluminescent material comprising the compound represented by Formula 1 according to claim 1.

8. An organic electroluminescent device comprising the compound represented by Formula 1 according to claim 1.

9. A plurality of host materials comprising a first host material comprising the compound according to claim 1, and a second host material comprising a compound represented by the following Formula 4: 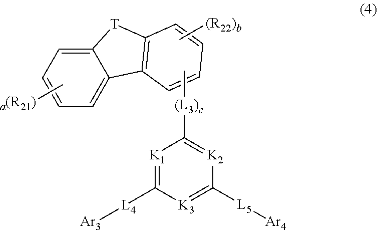
wherein in Formula 4,
T represents O or S;
K₁ to K₃, each independently, represent N or CR₂₃; with the proviso that at least one of K₁ to K₃ represents N;
L₃ to L₅, each independently, represent a single bond, a substituted or unsubstituted (C6-C30)arylene, a substituted or unsubstituted (C3-C30)cycloalkylene, or a substituted or unsubstituted (3- to 30-membered)heteroarylene;
R₂₁ to R₂₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), or —N—(R′)(R″); or may be linked to an adjacent substituent(s) to form a ring(s);
Ar₃ and Ar₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), or —N—(R′)(R″); or may be linked to an adjacent substituent(s) to form a ring(s);
R′ and R″, each independently, represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; and
a represents an integer of 1 to 4, b represents an integer of 1 to 3, c represents an integer of 1 or 2, and if a to c, respectively, represent an integer of 2 or more, each of R₂₁, each of R₂₂, and each of L₃ may be the same as or different from each other.

10. The plurality of host materials according to claim 9, wherein the compound represented by Formula 4 is represented by any one of the following Formulas 4-1 to 4-4: 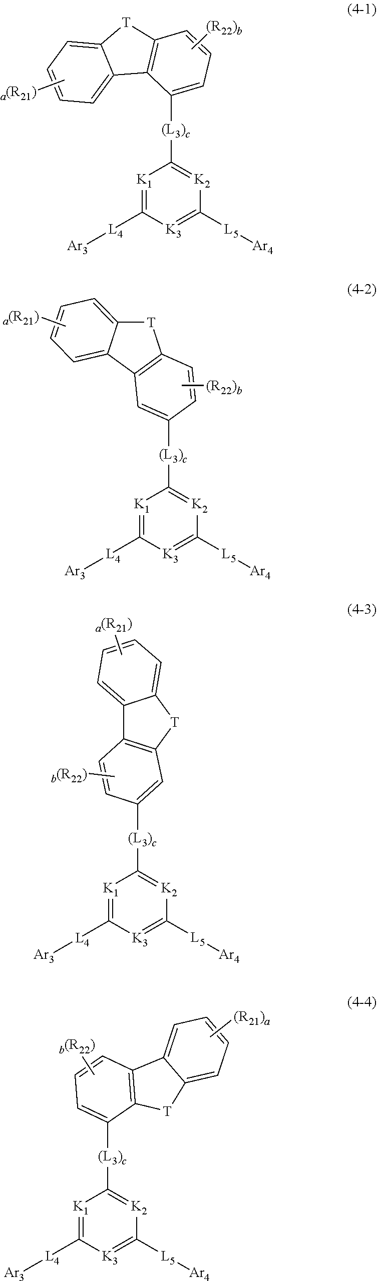
wherein in Formulas 4-1 to 4-4, T, K₁ to K₃, L₃ to L₅, R₂₁, R₂₂, Ar₃, Ar₄, a, b, and c are as defined in claim 9.

11. The plurality of host materials according to claim 9, wherein the compound represented by Formula 4 is at least one selected from the following compounds: 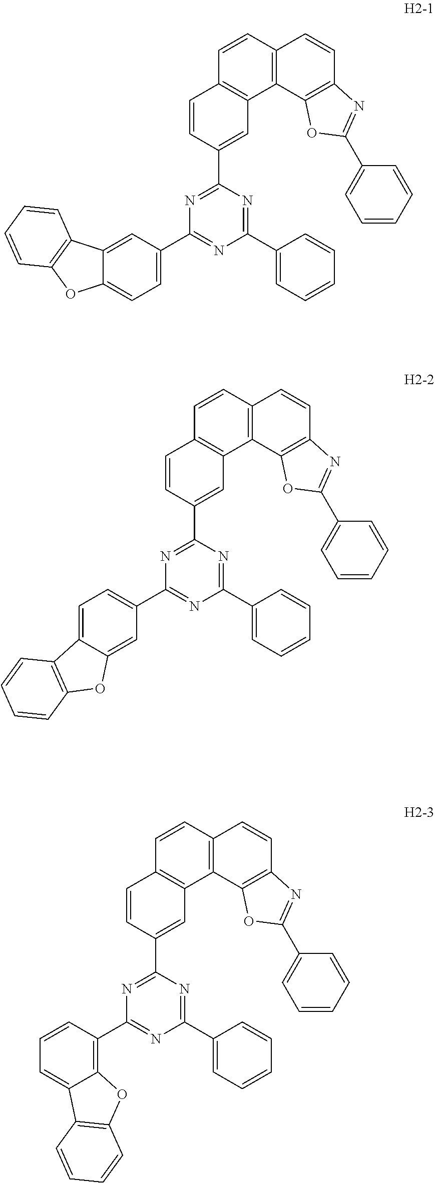 [SMILES: c1ccc(-c2nc(-c3ccc4oc5ccccc5c4c3)nc(-c3ccc4ccc5ccc6nc(-c7ccccc7)oc6c5c4c3)n2)cc1 ; c1ccc(-c2nc(-c3ccc4c(c3)oc3ccccc34)nc(-c3ccc4ccc5ccc6nc(-c7ccccc7)oc6c5c4c3)n2)cc1 ; c1ccc(-c2nc(-c3ccc4ccc5ccc6nc(-c7ccccc7)oc6c5c4c3)nc(-c3cccc4c3oc3ccccc34)n2)cc1] 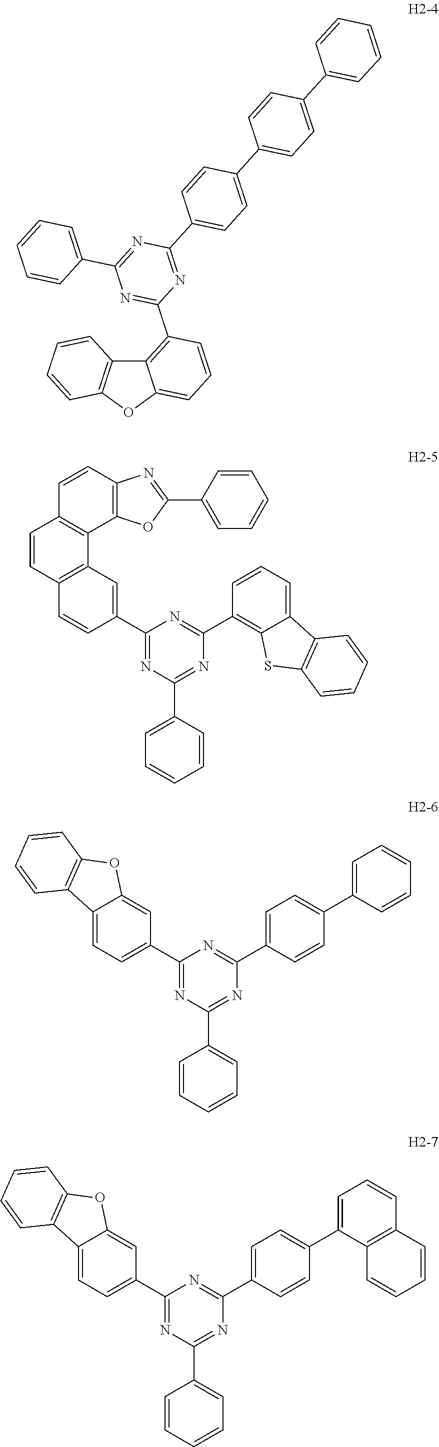 [SMILES: c1ccc(-c2ccc(-c3ccc(-c4nc(-c5ccccc5)nc(-c5cccc6oc7ccccc7c56)n4)cc3)cc2)cc1 ; c1ccc(-c2nc(-c3ccc4ccc5ccc6nc(-c7ccccc7)oc6c5c4c3)nc(-c3cccc4c3sc3ccccc34)n2)cc1 ; c1ccc(-c2ccc(-c3nc(-c4ccccc4)nc(-c4ccc5c(c4)oc4ccccc45)n3)cc2)cc1 ; c1ccc(-c2nc(-c3ccc(-c4cccc5ccccc45)cc3)nc(-c3ccc4c(c3)oc3ccccc34)n2)cc1] 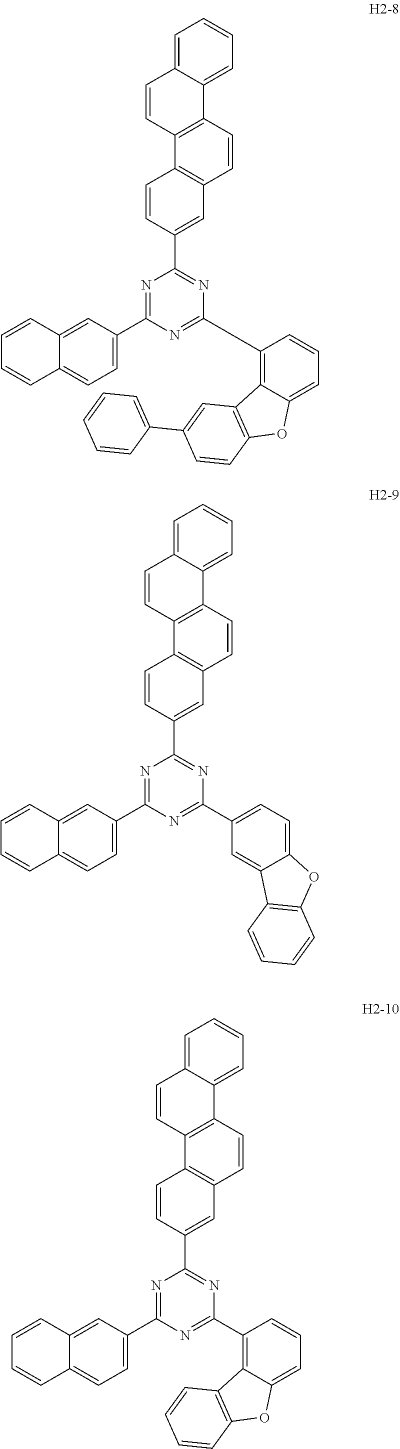 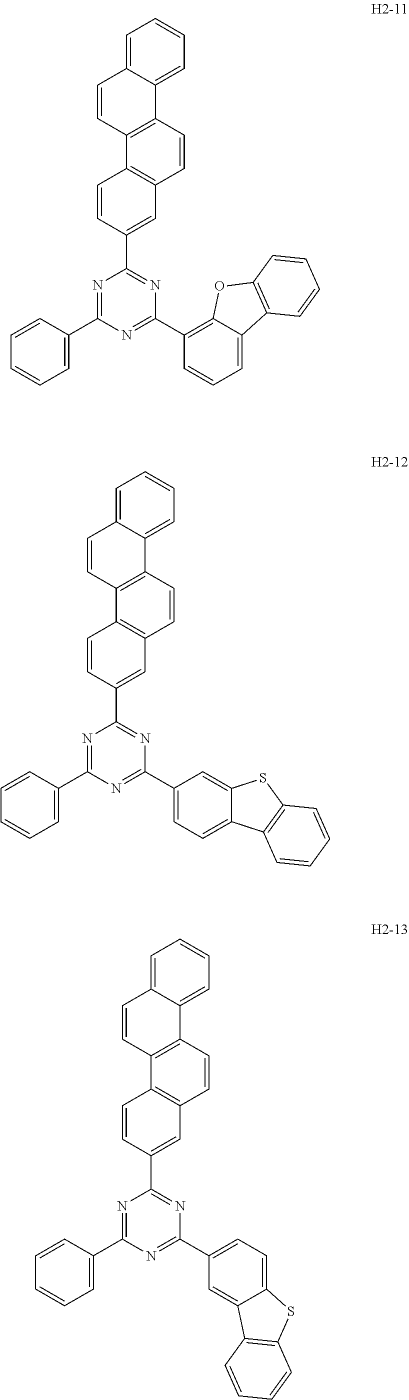 [SMILES: c1ccc(-c2nc(-c3ccc4c(ccc5c6ccccc6ccc45)c3)nc(-c3cccc4c3oc3ccccc34)n2)cc1 ; c1ccc(-c2nc(-c3ccc4c(ccc5c6ccccc6ccc45)c3)nc(-c3ccc4c(c3)sc3ccccc34)n2)cc1 ; c1ccc(-c2nc(-c3ccc4c(ccc5c6ccccc6ccc45)c3)nc(-c3ccc4sc5ccccc5c4c3)n2)cc1] 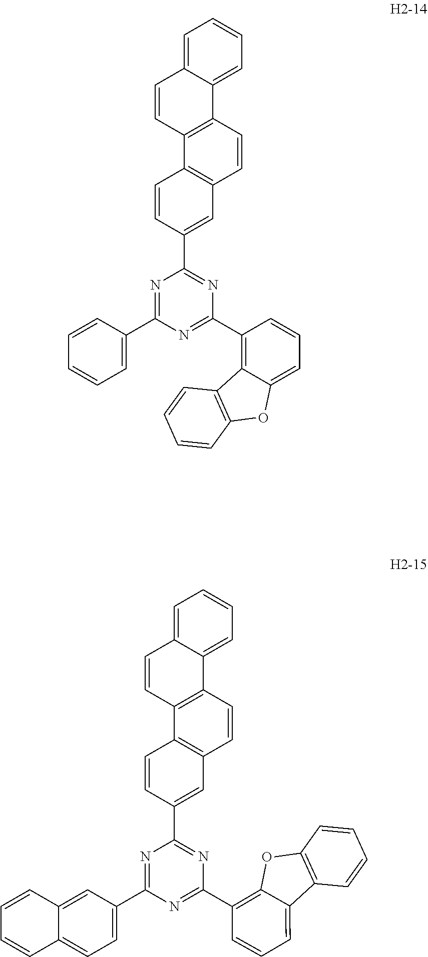 [SMILES: c1ccc(-c2nc(-c3ccc4c(ccc5c6ccccc6ccc45)c3)nc(-c3cccc4oc5ccccc5c34)n2)cc1 ; c1ccc2cc(-c3nc(-c4ccc5c(ccc6c7ccccc7ccc56)c4)nc(-c4cccc5c4oc4ccccc45)n3)ccc2c1] 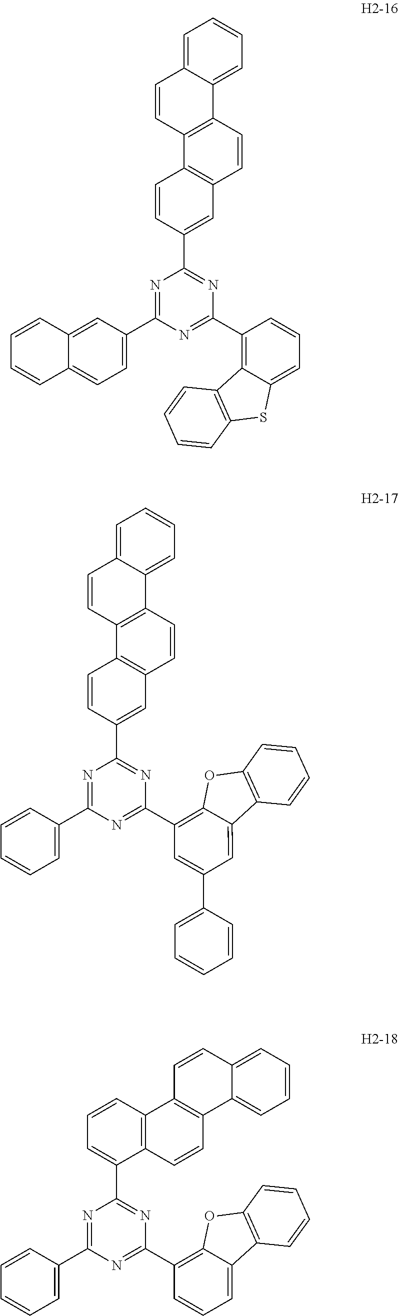 [SMILES: c1ccc2cc(-c3nc(-c4ccc5c(ccc6c7ccccc7ccc56)c4)nc(-c4cccc5sc6ccccc6c45)n3)ccc2c1 ; c1ccc(-c2cc(-c3nc(-c4ccccc4)nc(-c4ccc5c(ccc6c7ccccc7ccc56)c4)n3)c3oc4ccccc4c3c2)cc1 ; c1ccc(-c2nc(-c3cccc4c3ccc3c5ccccc5ccc43)nc(-c3cccc4c3oc3ccccc34)n2)cc1] 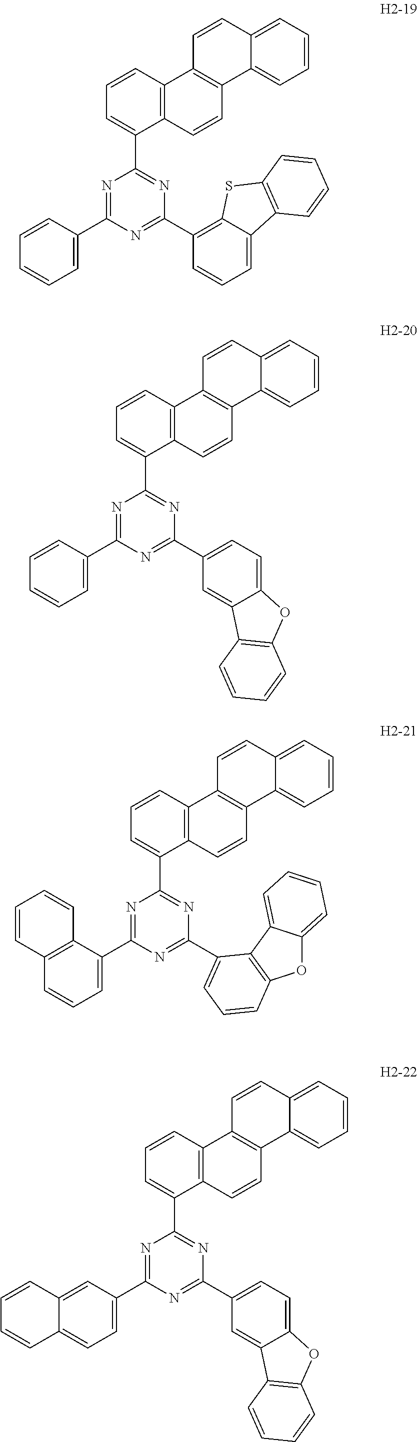 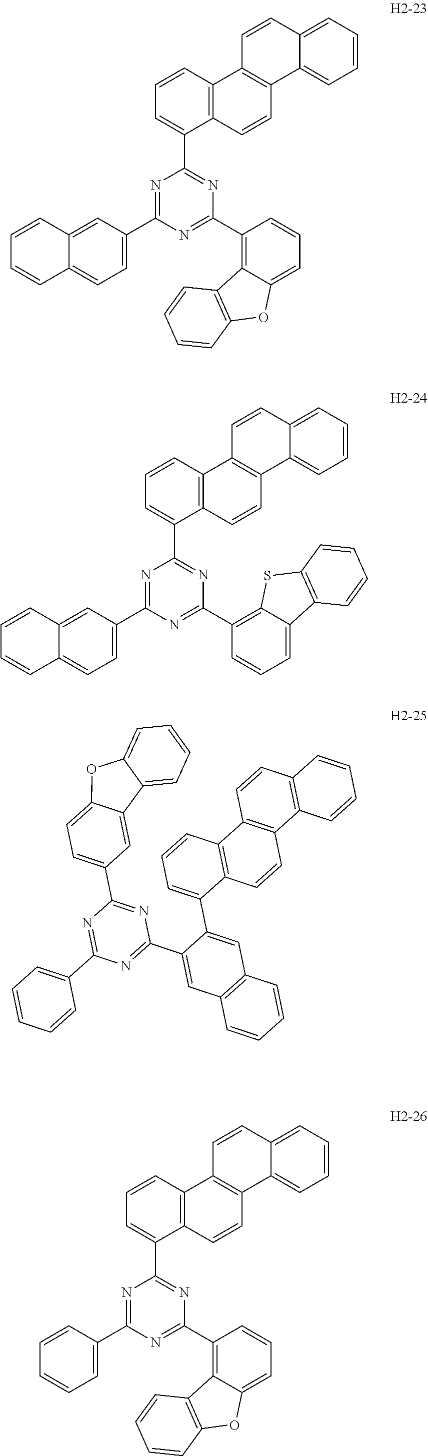 [SMILES: c1ccc2cc(-c3nc(-c4cccc5c4ccc4c6ccccc6ccc54)nc(-c4cccc5oc6ccccc6c45)n3)ccc2c1 ; c1ccc2cc(-c3nc(-c4cccc5c4ccc4c6ccccc6ccc54)nc(-c4cccc5c4sc4ccccc45)n3)ccc2c1 ; c1ccc(-c2nc(-c3ccc4oc5ccccc5c4c3)nc(-c3cc4ccccc4cc3-c3cccc4c3ccc3c5ccccc5ccc43)n2)cc1 ; c1ccc(-c2nc(-c3cccc4c3ccc3c5ccccc5ccc43)nc(-c3cccc4oc5ccccc5c34)n2)cc1] 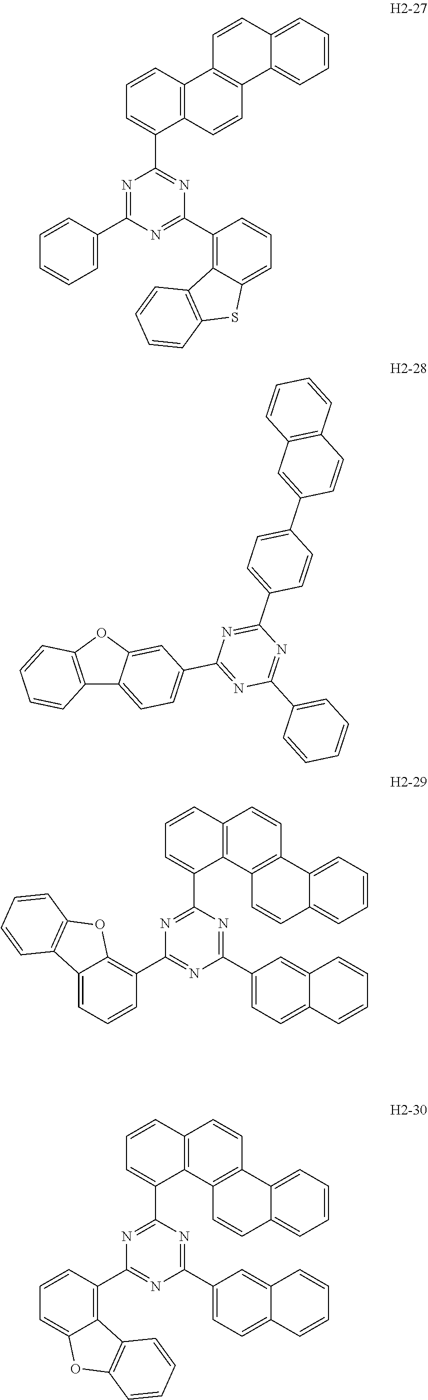 [SMILES: c1ccc(-c2nc(-c3cccc4c3ccc3c5ccccc5ccc43)nc(-c3cccc4sc5ccccc5c34)n2)cc1 ; c1ccc(-c2nc(-c3ccc(-c4ccc5ccccc5c4)cc3)nc(-c3ccc4c(c3)oc3ccccc34)n2)cc1 ; c1ccc2cc(-c3nc(-c4cccc5c4oc4ccccc45)nc(-c4cccc5ccc6c7ccccc7ccc6c45)n3)ccc2c1 ; c1ccc2cc(-c3nc(-c4cccc5ccc6c7ccccc7ccc6c45)nc(-c4cccc5oc6ccccc6c45)n3)ccc2c1] 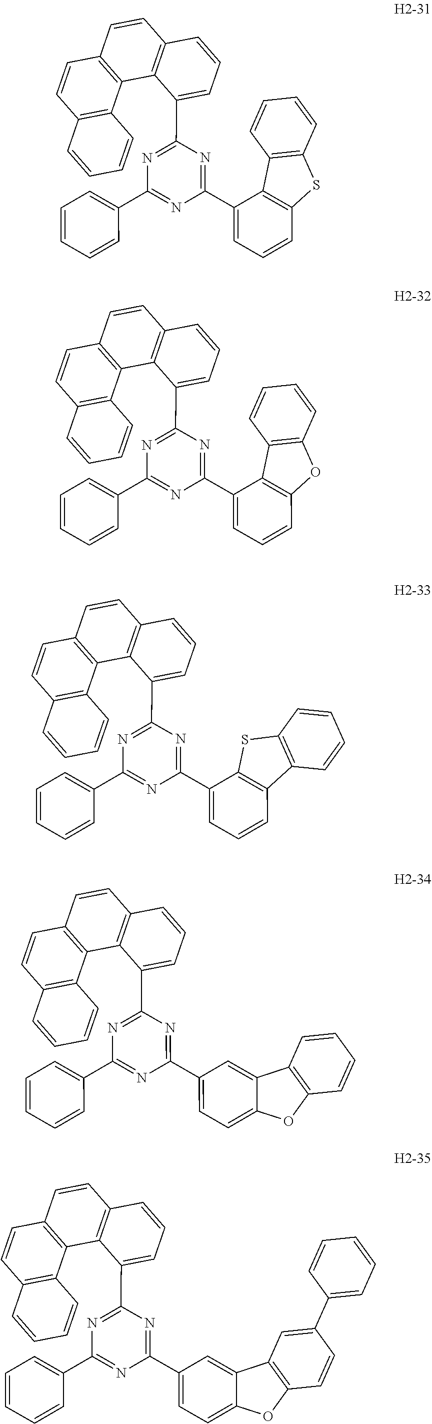 [SMILES: c1ccc(-c2nc(-c3cccc4sc5ccccc5c34)nc(-c3cccc4ccc5ccc6ccccc6c5c34)n2)cc1 ; c1ccc(-c2nc(-c3cccc4oc5ccccc5c34)nc(-c3cccc4ccc5ccc6ccccc6c5c34)n2)cc1 ; c1ccc(-c2nc(-c3cccc4c3sc3ccccc34)nc(-c3cccc4ccc5ccc6ccccc6c5c34)n2)cc1 ; c1ccc(-c2nc(-c3ccc4oc5ccccc5c4c3)nc(-c3cccc4ccc5ccc6ccccc6c5c34)n2)cc1 ; c1ccc(-c2ccc3oc4ccc(-c5nc(-c6ccccc6)nc(-c6cccc7ccc8ccc9ccccc9c8c67)n5)cc4c3c2)cc1] 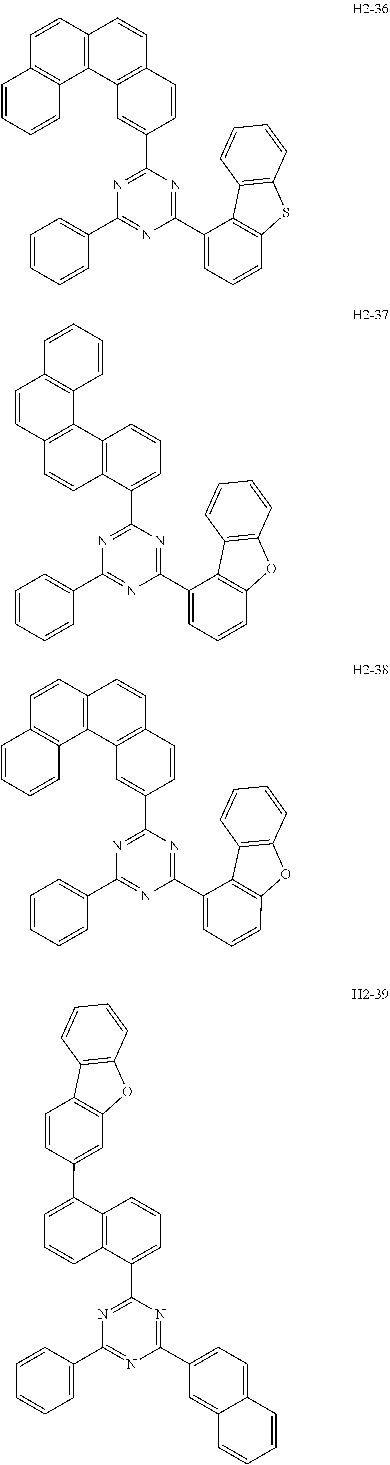 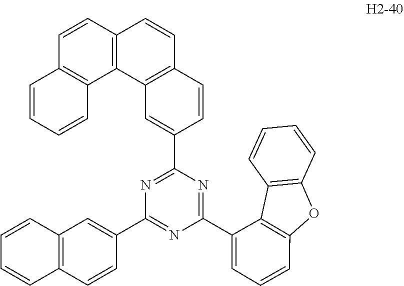 [SMILES: c1ccc2cc(-c3nc(-c4ccc5ccc6ccc7ccccc7c6c5c4)nc(-c4cccc5oc6ccccc6c45)n3)ccc2c1] 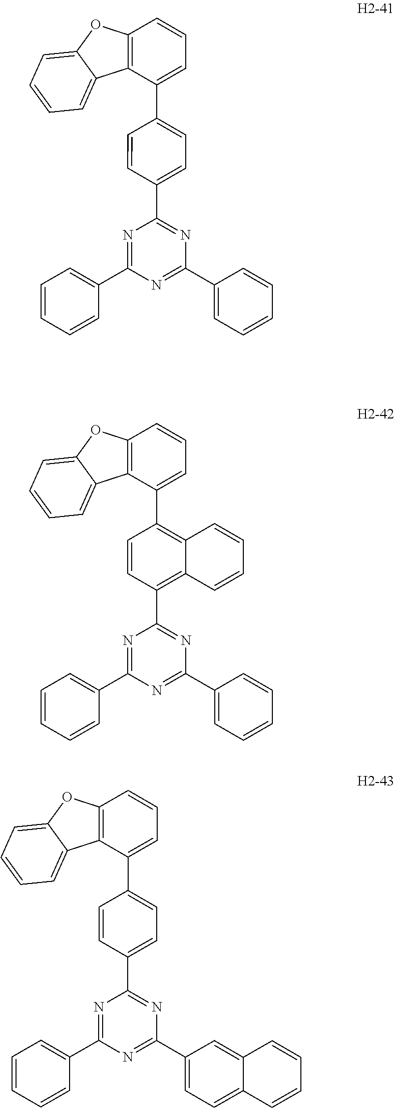 [SMILES: c1ccc(-c2nc(-c3ccccc3)nc(-c3ccc(-c4cccc5oc6ccccc6c45)cc3)n2)cc1 ; c1ccc(-c2nc(-c3ccccc3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccccc4c3)n2)cc1] 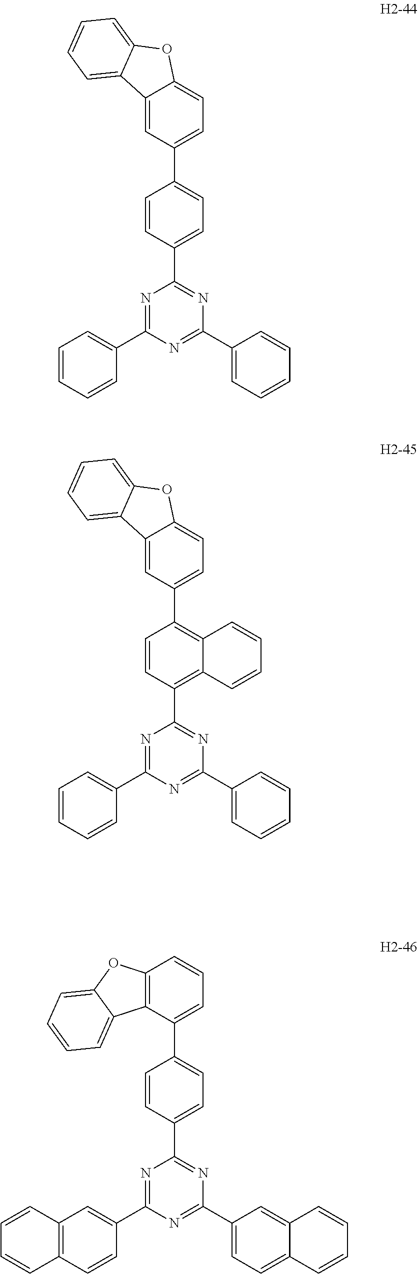 [SMILES: c1ccc(-c2nc(-c3ccccc3)nc(-c3ccc(-c4ccc5oc6ccccc6c5c4)cc3)n2)cc1 ; c1ccc(-c2nc(-c3ccccc3)nc(-c3ccc(-c4ccc5oc6ccccc6c5c4)c4ccccc34)n2)cc1 ; c1ccc2cc(-c3nc(-c4ccc(-c5cccc6oc7ccccc7c56)cc4)nc(-c4ccc5ccccc5c4)n3)ccc2c1] 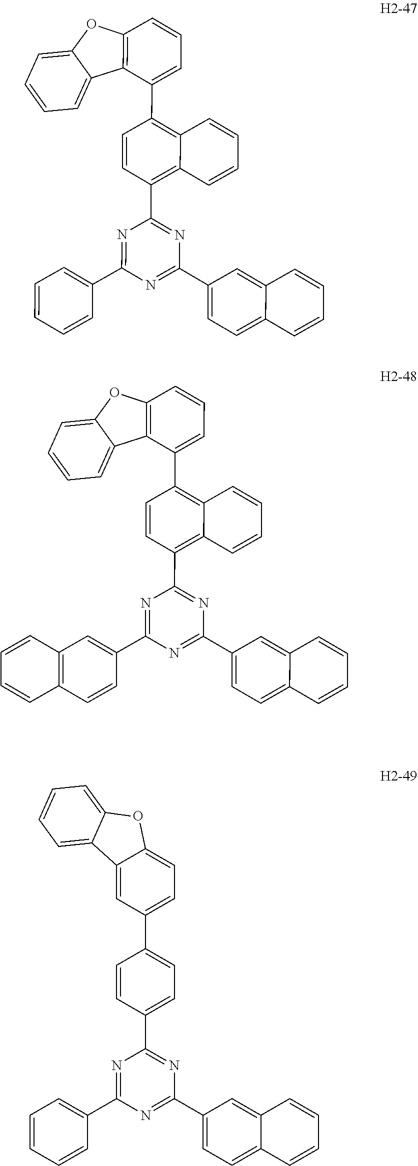 [SMILES: c1ccc(-c2nc(-c3ccc4ccccc4c3)nc(-c3ccc(-c4cccc5oc6ccccc6c45)c4ccccc34)n2)cc1 ; c1ccc2cc(-c3nc(-c4ccc5ccccc5c4)nc(-c4ccc(-c5cccc6oc7ccccc7c56)c5ccccc45)n3)ccc2c1 ; c1ccc(-c2nc(-c3ccc(-c4ccc5oc6ccccc6c5c4)cc3)nc(-c3ccc4ccccc4c3)n2)cc1] 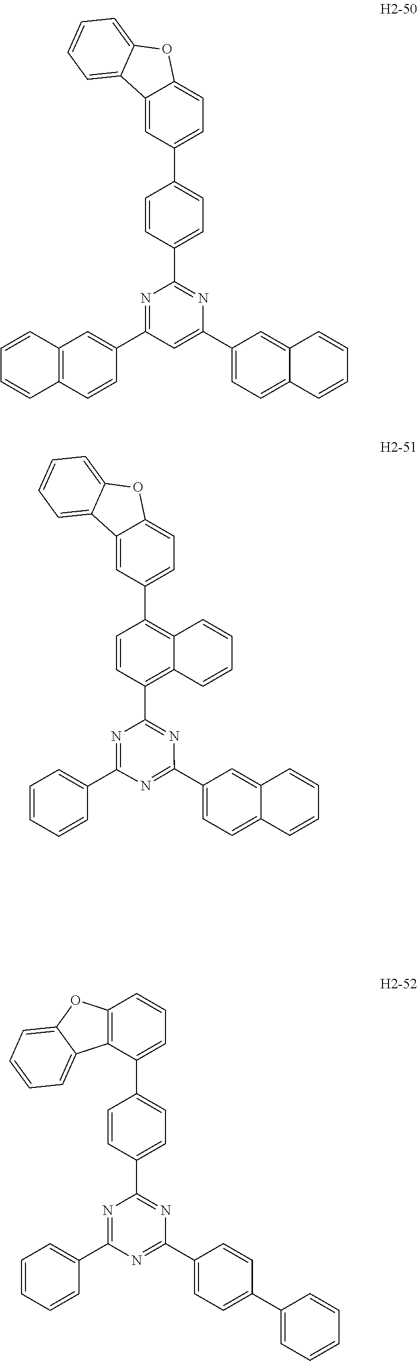 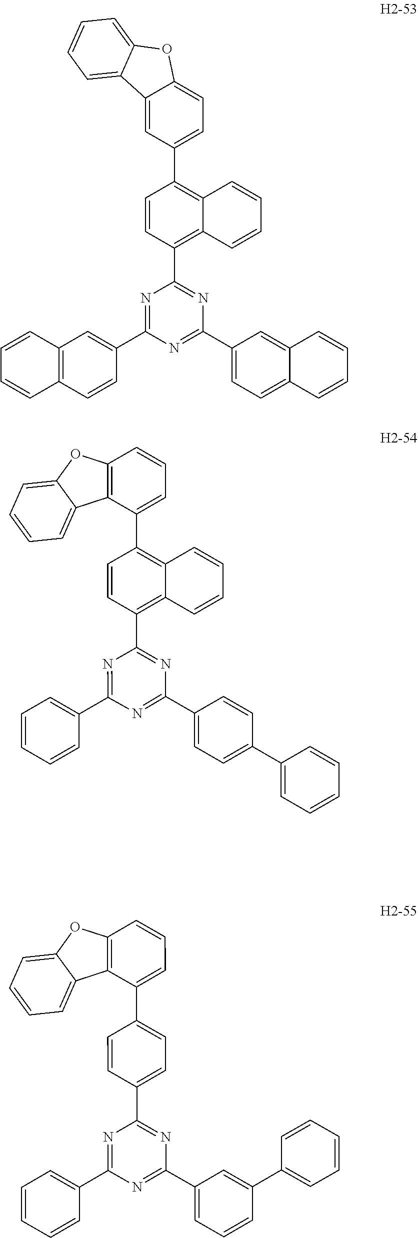 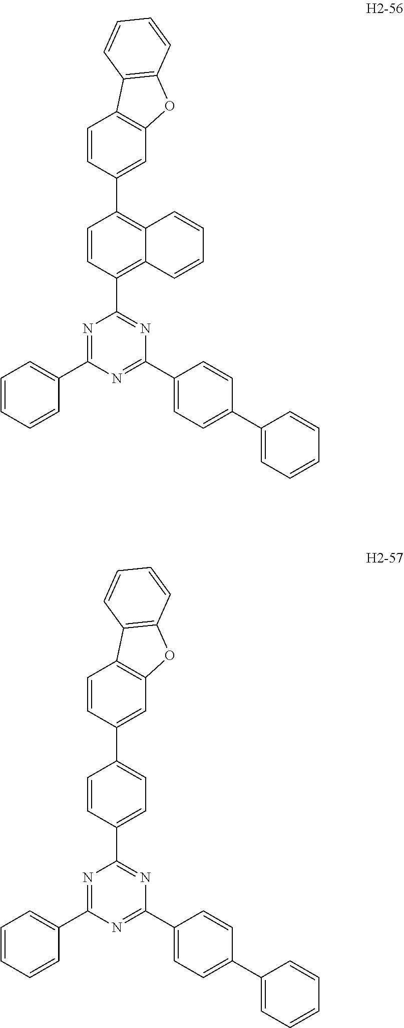 [SMILES: c1ccc(-c2ccc(-c3nc(-c4ccccc4)nc(-c4ccc(-c5ccc6c(c5)oc5ccccc56)c5ccccc45)n3)cc2)cc1 ; c1ccc(-c2ccc(-c3nc(-c4ccccc4)nc(-c4ccc(-c5ccc6c(c5)oc5ccccc56)cc4)n3)cc2)cc1] 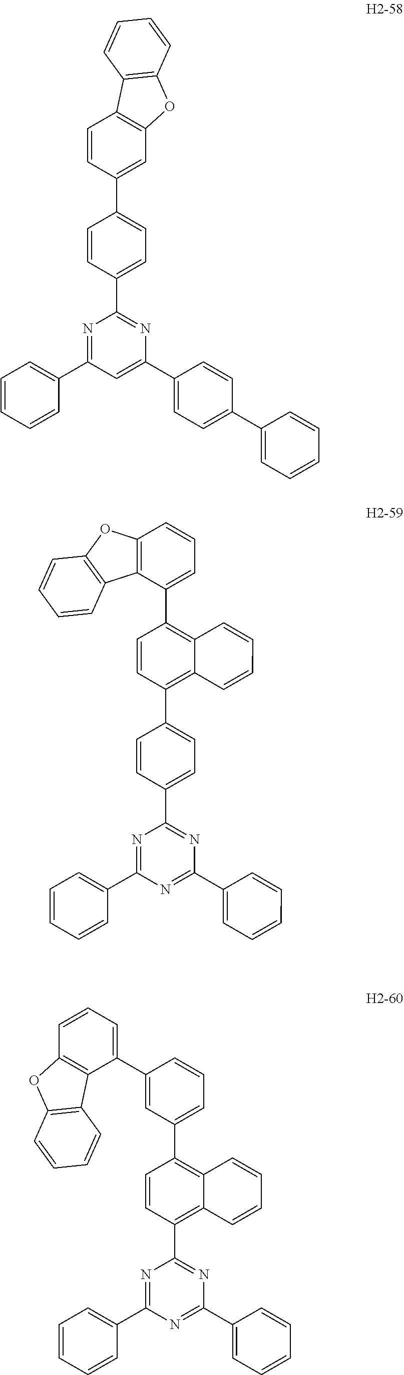 [SMILES: c1ccc(-c2ccc(-c3cc(-c4ccccc4)nc(-c4ccc(-c5ccc6c(c5)oc5ccccc56)cc4)n3)cc2)cc1 ; c1ccc(-c2nc(-c3ccccc3)nc(-c3ccc(-c4ccc(-c5cccc6oc7ccccc7c56)c5ccccc45)cc3)n2)cc1 ; c1ccc(-c2nc(-c3ccccc3)nc(-c3ccc(-c4cccc(-c5cccc6oc7ccccc7c56)c4)c4ccccc34)n2)cc1] 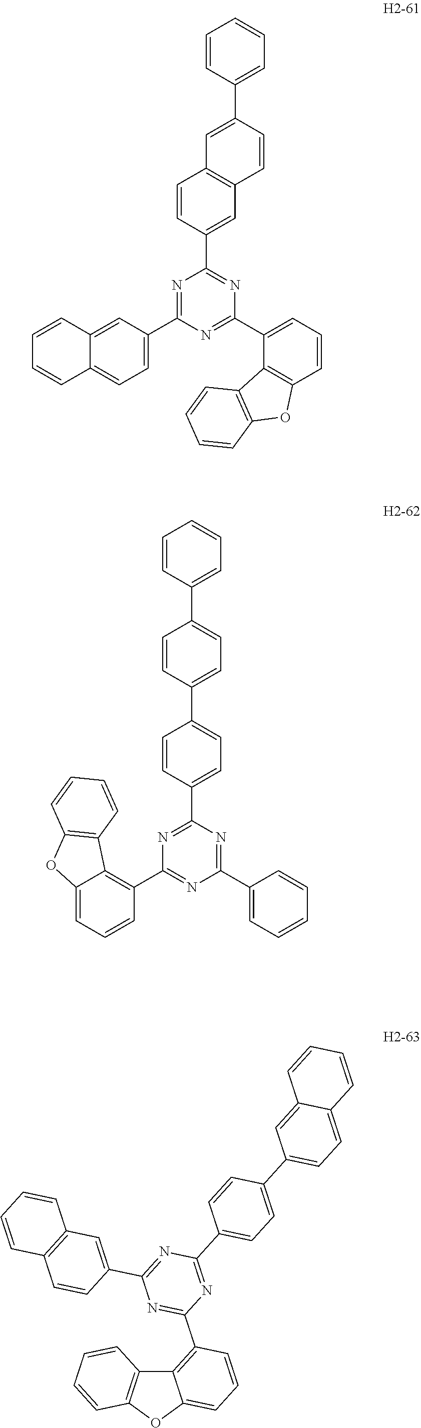 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccccc5)nc(-c5cccc6oc7ccccc7c56)n4)cc3)cc2)cc1 ; c1ccc2cc(-c3ccc(-c4nc(-c5ccc6ccccc6c5)nc(-c5cccc6oc7ccccc7c56)n4)cc3)ccc2c1] 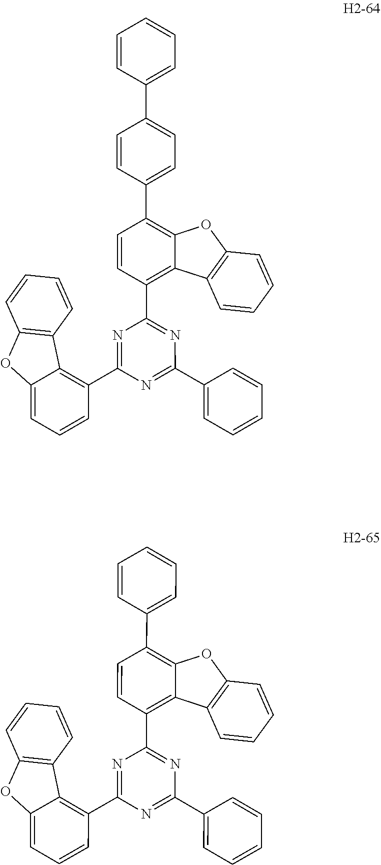 [SMILES: c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2nc(-c3cccc4oc5ccccc5c34)nc(-c3ccc(-c4ccccc4)c4oc5ccccc5c34)n2)cc1] 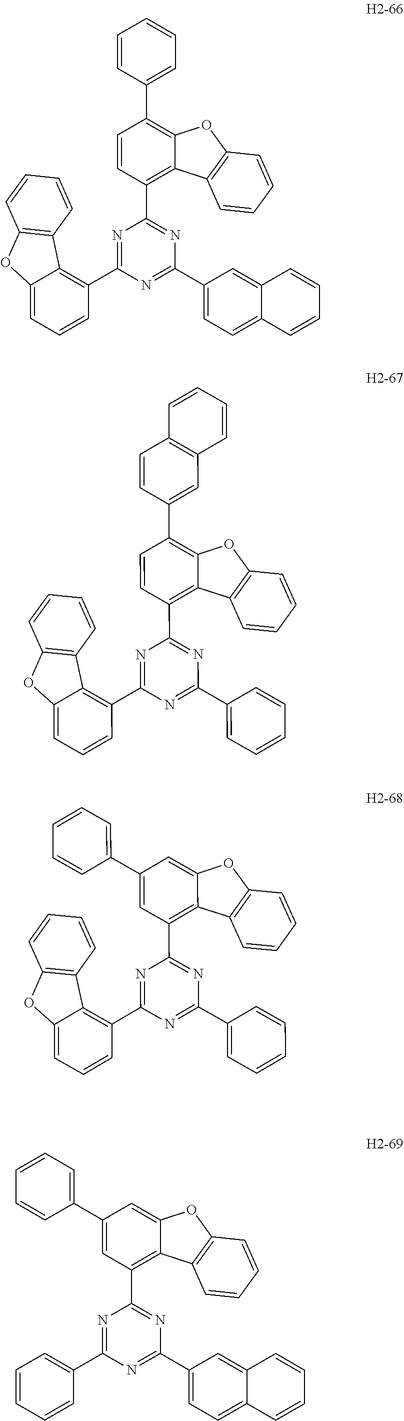 [SMILES: c1ccc(-c2ccc(-c3nc(-c4ccc5ccccc5c4)nc(-c4cccc5oc6ccccc6c45)n3)c3c2oc2ccccc23)cc1 ; c1ccc(-c2nc(-c3cccc4oc5ccccc5c34)nc(-c3ccc(-c4ccc5ccccc5c4)c4oc5ccccc5c34)n2)cc1 ; c1ccc(-c2cc(-c3nc(-c4ccccc4)nc(-c4cccc5oc6ccccc6c45)n3)c3c(c2)oc2ccccc23)cc1 ; c1ccc(-c2cc(-c3nc(-c4ccccc4)nc(-c4ccc5ccccc5c4)n3)c3c(c2)oc2ccccc23)cc1] 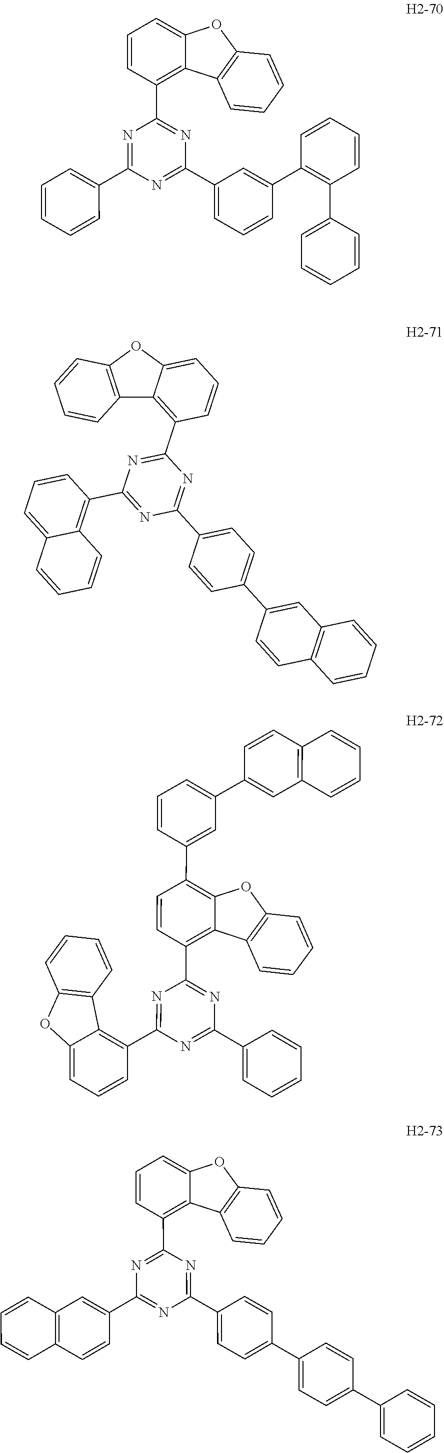 [SMILES: c1ccc(-c2nc(-c3cccc(-c4ccccc4-c4ccccc4)c3)nc(-c3cccc4oc5ccccc5c34)n2)cc1 ; c1ccc2cc(-c3ccc(-c4nc(-c5cccc6ccccc56)nc(-c5cccc6oc7ccccc7c56)n4)cc3)ccc2c1 ; c1ccc(-c2nc(-c3cccc4oc5ccccc5c34)nc(-c3ccc(-c4cccc(-c5ccc6ccccc6c5)c4)c4oc5ccccc5c34)n2)cc1 ; c1ccc(-c2ccc(-c3ccc(-c4nc(-c5ccc6ccccc6c5)nc(-c5cccc6oc7ccccc7c56)n4)cc3)cc2)cc1] 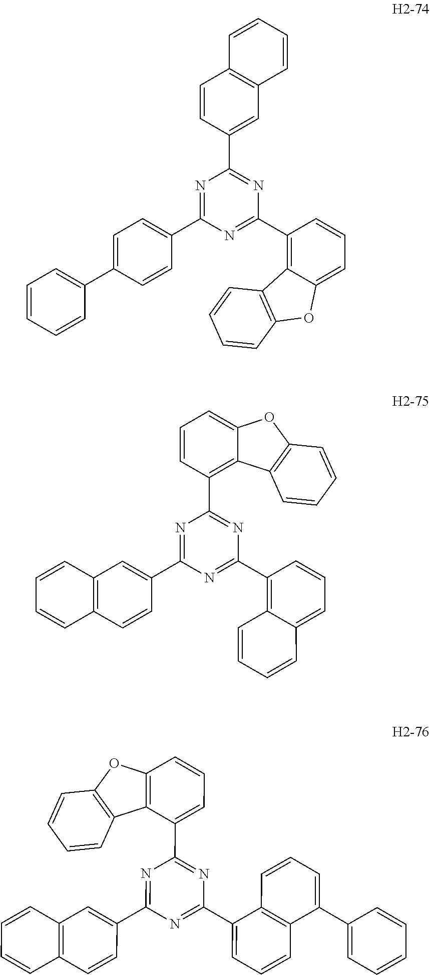 [SMILES: c1ccc(-c2ccc(-c3nc(-c4ccc5ccccc5c4)nc(-c4cccc5oc6ccccc6c45)n3)cc2)cc1 ; c1ccc2cc(-c3nc(-c4cccc5ccccc45)nc(-c4cccc5oc6ccccc6c45)n3)ccc2c1 ; c1ccc(-c2cccc3c(-c4nc(-c5ccc6ccccc6c5)nc(-c5cccc6oc7ccccc7c56)n4)cccc23)cc1] 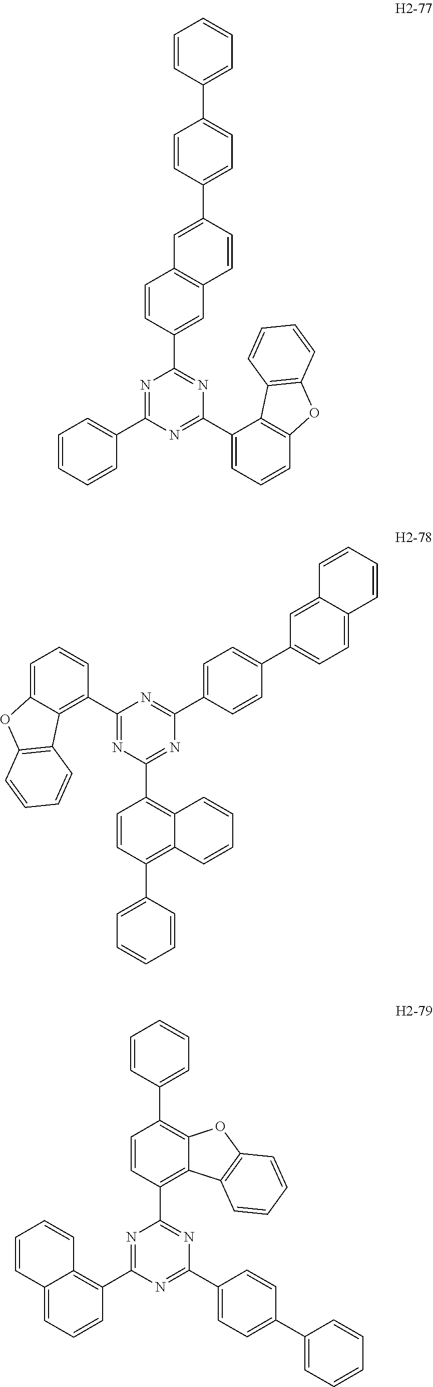 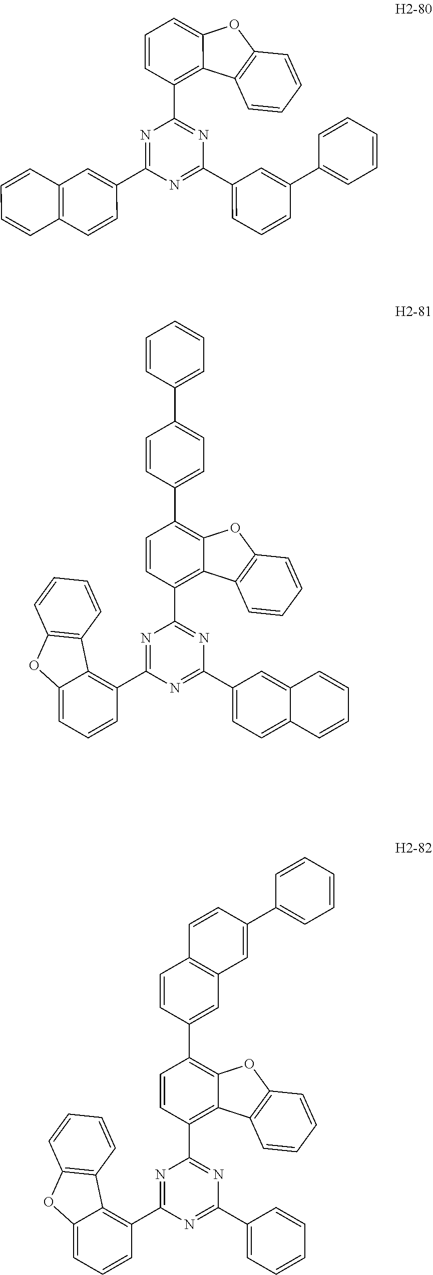 [SMILES: c1ccc(-c2cccc(-c3nc(-c4ccc5ccccc5c4)nc(-c4cccc5oc6ccccc6c45)n3)c2)cc1 ; c1ccc(-c2ccc(-c3ccc(-c4nc(-c5ccc6ccccc6c5)nc(-c5cccc6oc7ccccc7c56)n4)c4c3oc3ccccc34)cc2)cc1 ; c1ccc(-c2ccc3ccc(-c4ccc(-c5nc(-c6ccccc6)nc(-c6cccc7oc8ccccc8c67)n5)c5c4oc4ccccc45)cc3c2)cc1] 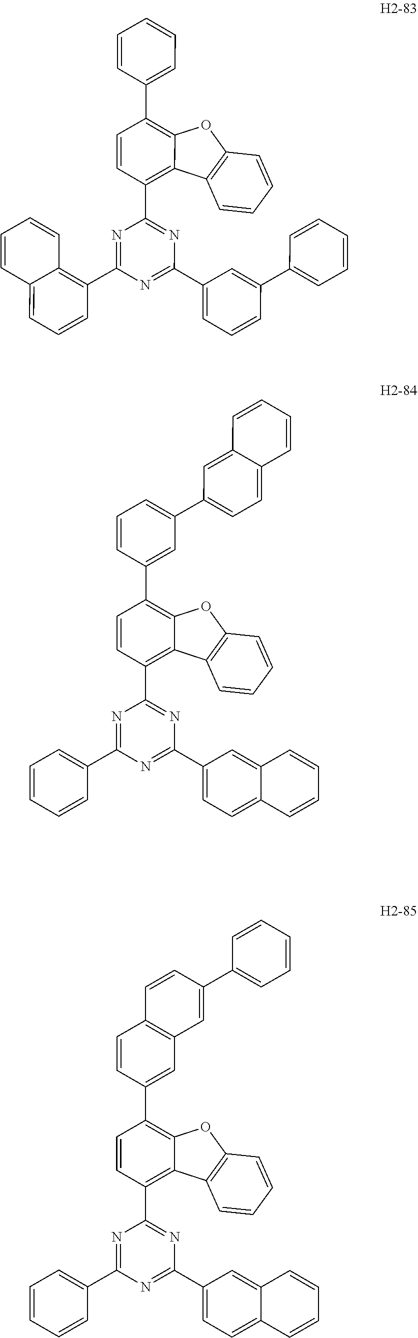 [SMILES: c1ccc(-c2cccc(-c3nc(-c4cccc5ccccc45)nc(-c4ccc(-c5ccccc5)c5oc6ccccc6c45)n3)c2)cc1 ; c1ccc(-c2nc(-c3ccc4ccccc4c3)nc(-c3ccc(-c4cccc(-c5ccc6ccccc6c5)c4)c4oc5ccccc5c34)n2)cc1 ; c1ccc(-c2ccc3ccc(-c4ccc(-c5nc(-c6ccccc6)nc(-c6ccc7ccccc7c6)n5)c5c4oc4ccccc45)cc3c2)cc1] 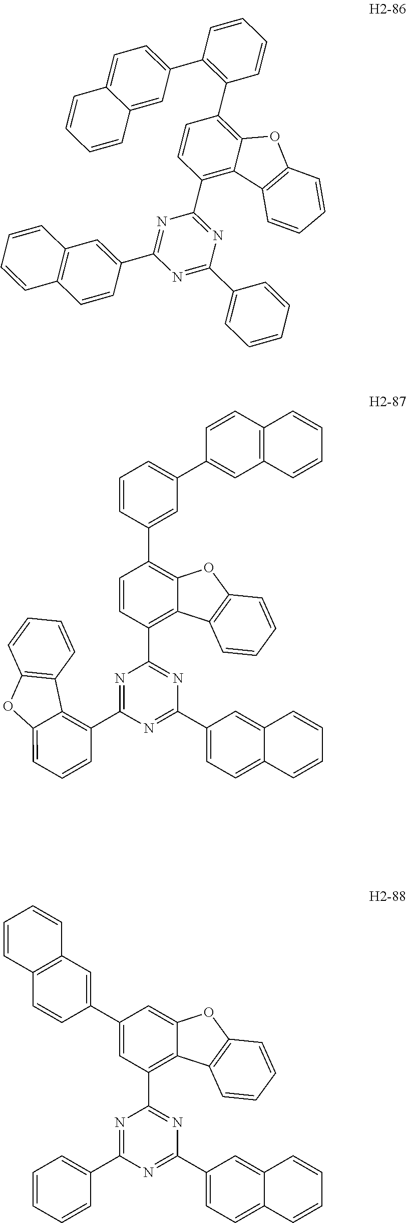 [SMILES: c1ccc(-c2nc(-c3ccc4ccccc4c3)nc(-c3ccc(-c4ccccc4-c4ccc5ccccc5c4)c4oc5ccccc5c34)n2)cc1 ; c1cc(-c2ccc3ccccc3c2)cc(-c2ccc(-c3nc(-c4ccc5ccccc5c4)nc(-c4cccc5oc6ccccc6c45)n3)c3c2oc2ccccc23)c1 ; c1ccc(-c2nc(-c3ccc4ccccc4c3)nc(-c3cc(-c4ccc5ccccc5c4)cc4oc5ccccc5c34)n2)cc1] 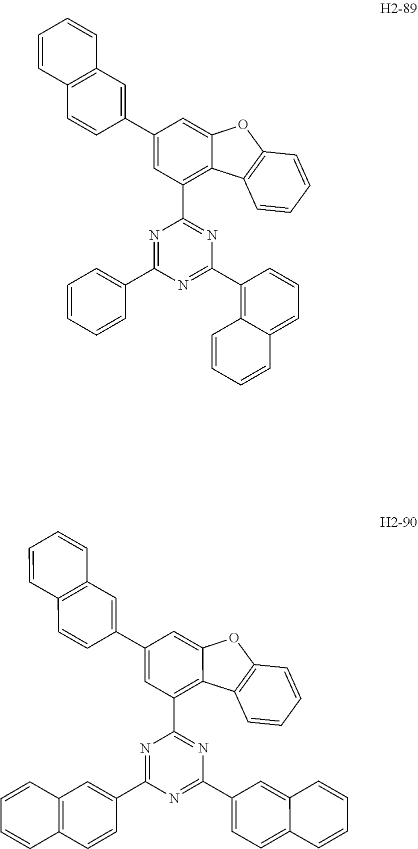 [SMILES: c1ccc(-c2nc(-c3cccc4ccccc34)nc(-c3cc(-c4ccc5ccccc5c4)cc4oc5ccccc5c34)n2)cc1 ; c1ccc2cc(-c3cc(-c4nc(-c5ccc6ccccc6c5)nc(-c5ccc6ccccc6c5)n4)c4c(c3)oc3ccccc34)ccc2c1] 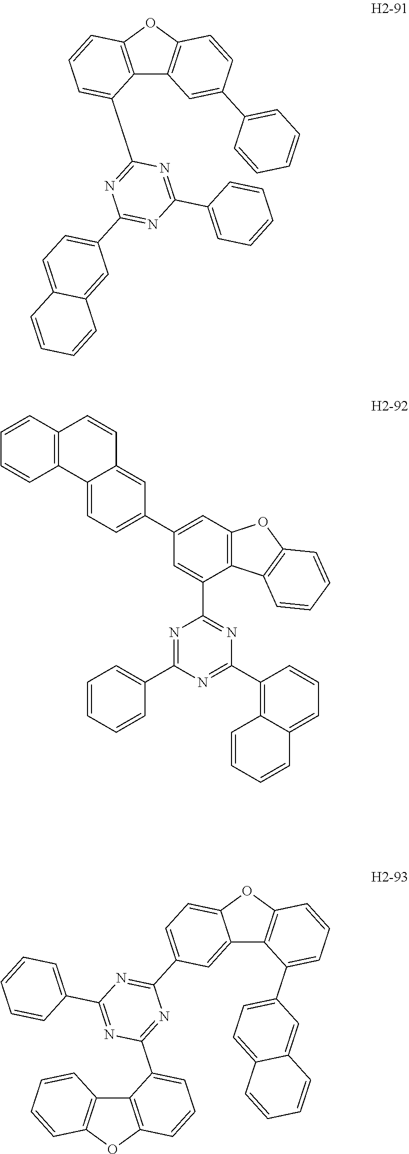 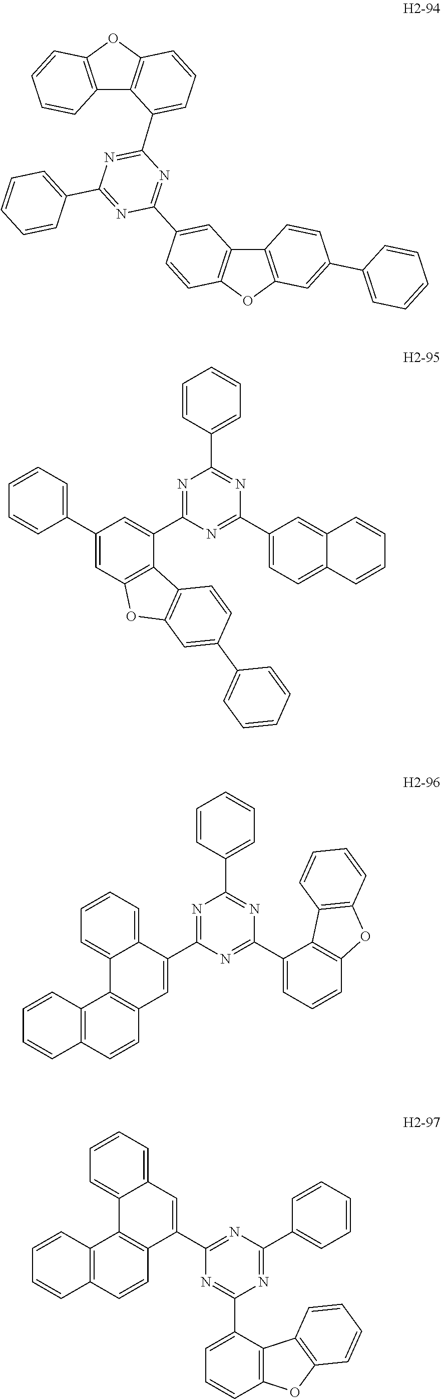 [SMILES: c1ccc(-c2ccc3c(c2)oc2ccc(-c4nc(-c5ccccc5)nc(-c5cccc6oc7ccccc7c56)n4)cc23)cc1 ; c1ccc(-c2ccc3c(c2)oc2cc(-c4ccccc4)cc(-c4nc(-c5ccccc5)nc(-c5ccc6ccccc6c5)n4)c23)cc1 ; c1ccc(-c2nc(-c3cc4ccc5ccccc5c4c4ccccc34)nc(-c3cccc4oc5ccccc5c34)n2)cc1 ; c1ccc(-c2nc(-c3cc4ccccc4c4c3ccc3ccccc34)nc(-c3cccc4oc5ccccc5c34)n2)cc1] 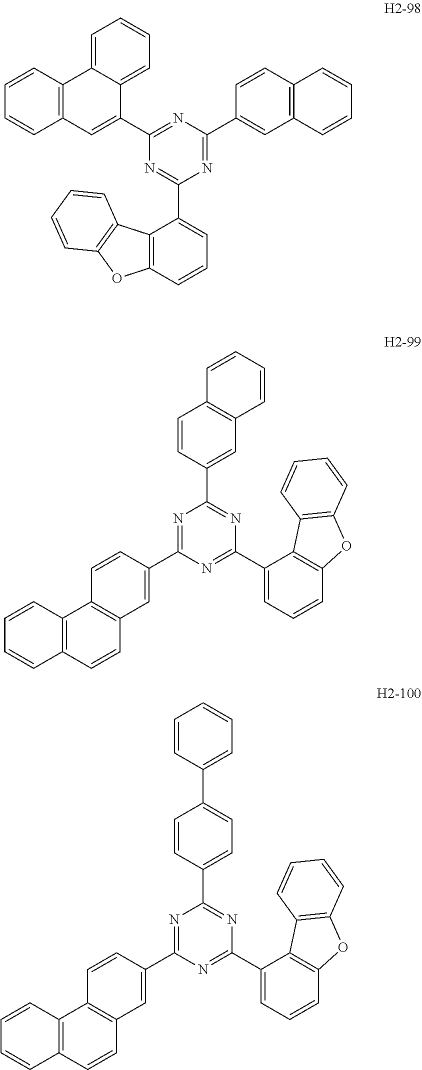 [SMILES: c1ccc2cc(-c3nc(-c4cc5ccccc5c5ccccc45)nc(-c4cccc5oc6ccccc6c45)n3)ccc2c1 ; c1ccc2cc(-c3nc(-c4ccc5c(ccc6ccccc65)c4)nc(-c4cccc5oc6ccccc6c45)n3)ccc2c1 ; c1ccc(-c2ccc(-c3nc(-c4ccc5c(ccc6ccccc65)c4)nc(-c4cccc5oc6ccccc6c45)n3)cc2)cc1] 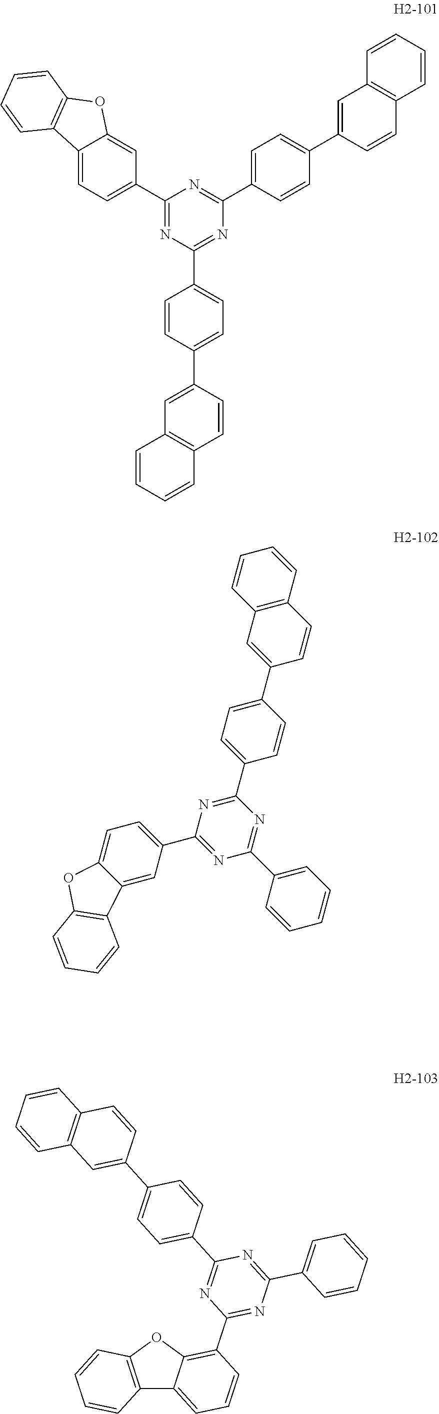 [SMILES: c1ccc2cc(-c3ccc(-c4nc(-c5ccc(-c6ccc7ccccc7c6)cc5)nc(-c5ccc6c(c5)oc5ccccc56)n4)cc3)ccc2c1 ; c1ccc(-c2nc(-c3ccc(-c4ccc5ccccc5c4)cc3)nc(-c3ccc4oc5ccccc5c4c3)n2)cc1 ; c1ccc(-c2nc(-c3ccc(-c4ccc5ccccc5c4)cc3)nc(-c3cccc4c3oc3ccccc34)n2)cc1] 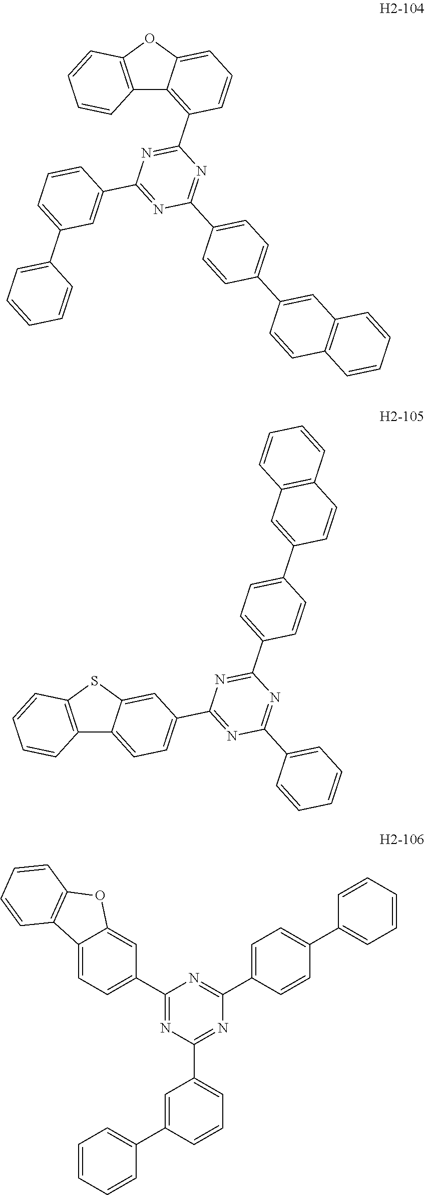 [SMILES: c1ccc(-c2cccc(-c3nc(-c4ccc(-c5ccc6ccccc6c5)cc4)nc(-c4cccc5oc6ccccc6c45)n3)c2)cc1 ; c1ccc(-c2nc(-c3ccc(-c4ccc5ccccc5c4)cc3)nc(-c3ccc4c(c3)sc3ccccc34)n2)cc1 ; c1ccc(-c2ccc(-c3nc(-c4cccc(-c5ccccc5)c4)nc(-c4ccc5c(c4)oc4ccccc45)n3)cc2)cc1] 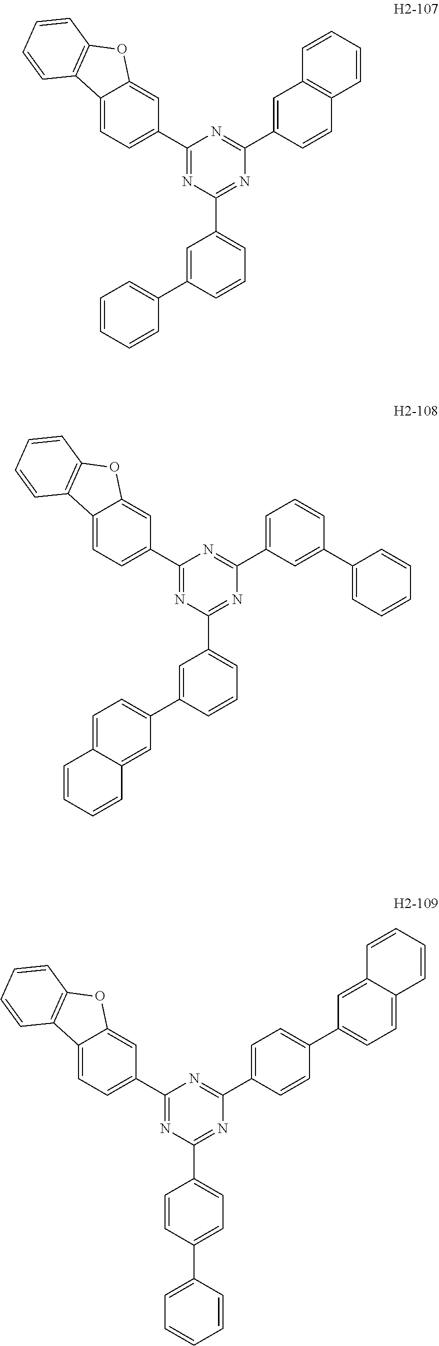 [SMILES: c1ccc(-c2cccc(-c3nc(-c4ccc5ccccc5c4)nc(-c4ccc5c(c4)oc4ccccc45)n3)c2)cc1 ; c1ccc(-c2cccc(-c3nc(-c4cccc(-c5ccc6ccccc6c5)c4)nc(-c4ccc5c(c4)oc4ccccc45)n3)c2)cc1 ; c1ccc(-c2ccc(-c3nc(-c4ccc(-c5ccc6ccccc6c5)cc4)nc(-c4ccc5c(c4)oc4ccccc45)n3)cc2)cc1] 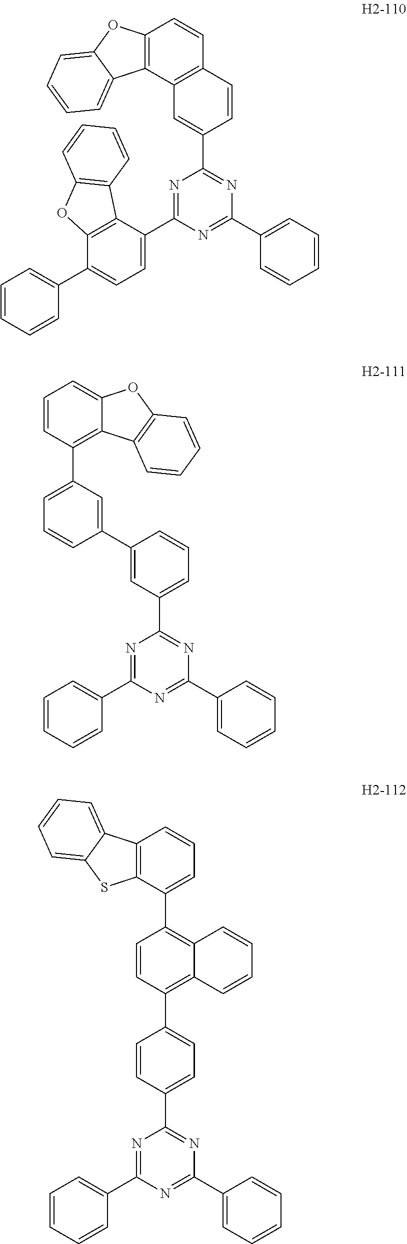 [SMILES: c1ccc(-c2nc(-c3ccc4ccc5oc6ccccc6c5c4c3)nc(-c3ccc(-c4ccccc4)c4oc5ccccc5c34)n2)cc1 ; c1ccc(-c2nc(-c3ccccc3)nc(-c3cccc(-c4cccc(-c5cccc6oc7ccccc7c56)c4)c3)n2)cc1 ; c1ccc(-c2nc(-c3ccccc3)nc(-c3ccc(-c4ccc(-c5cccc6c5sc5ccccc56)c5ccccc45)cc3)n2)cc1] 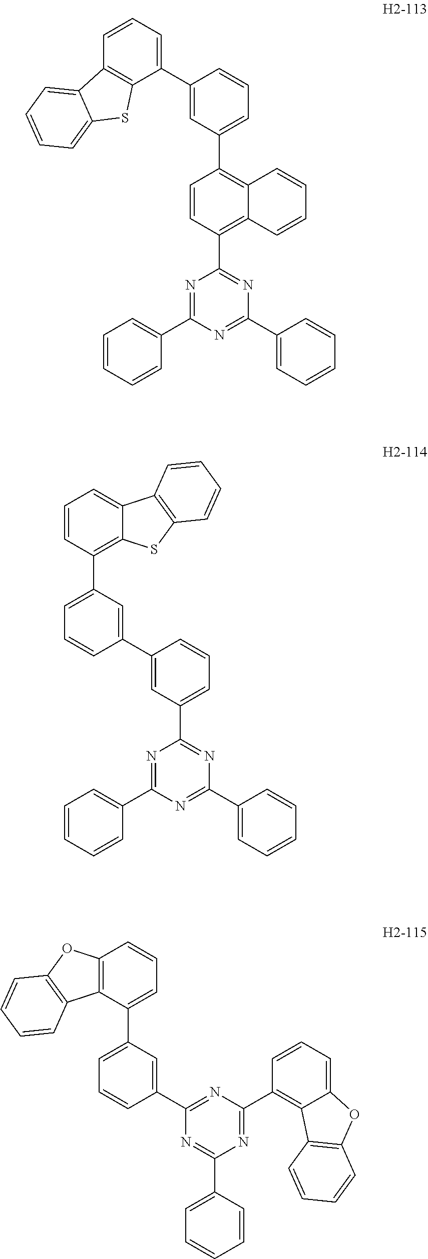 [SMILES: c1ccc(-c2nc(-c3ccccc3)nc(-c3ccc(-c4cccc(-c5cccc6c5sc5ccccc56)c4)c4ccccc34)n2)cc1 ; c1ccc(-c2nc(-c3ccccc3)nc(-c3cccc(-c4cccc(-c5cccc6c5sc5ccccc56)c4)c3)n2)cc1 ; c1ccc(-c2nc(-c3cccc(-c4cccc5oc6ccccc6c45)c3)nc(-c3cccc4oc5ccccc5c34)n2)cc1] 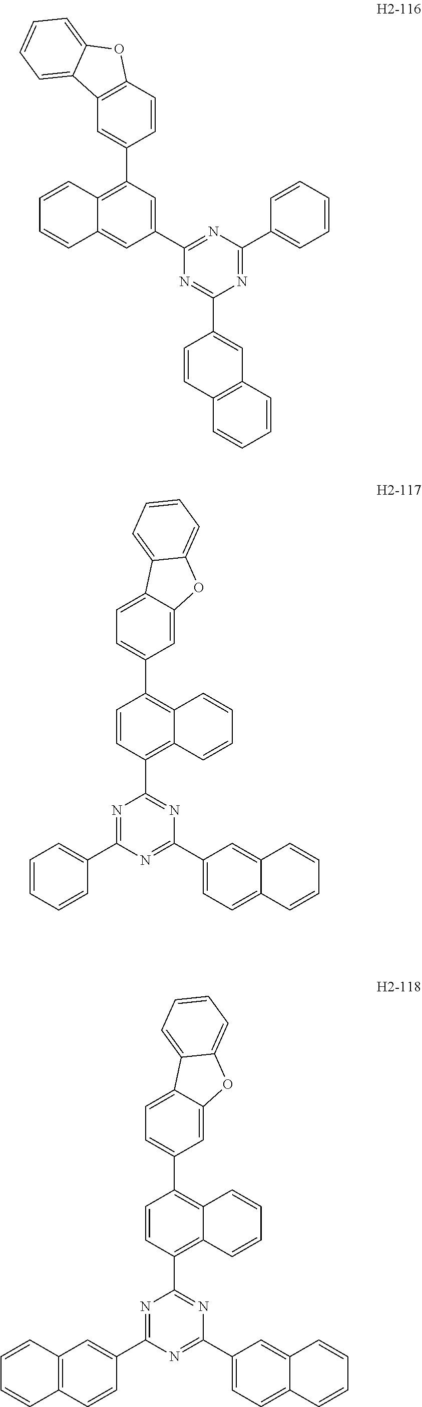 [SMILES: c1ccc(-c2nc(-c3ccc4ccccc4c3)nc(-c3cc(-c4ccc5oc6ccccc6c5c4)c4ccccc4c3)n2)cc1 ; c1ccc(-c2nc(-c3ccc4ccccc4c3)nc(-c3ccc(-c4ccc5c(c4)oc4ccccc45)c4ccccc34)n2)cc1 ; c1ccc2cc(-c3nc(-c4ccc5ccccc5c4)nc(-c4ccc(-c5ccc6c(c5)oc5ccccc56)c5ccccc45)n3)ccc2c1] 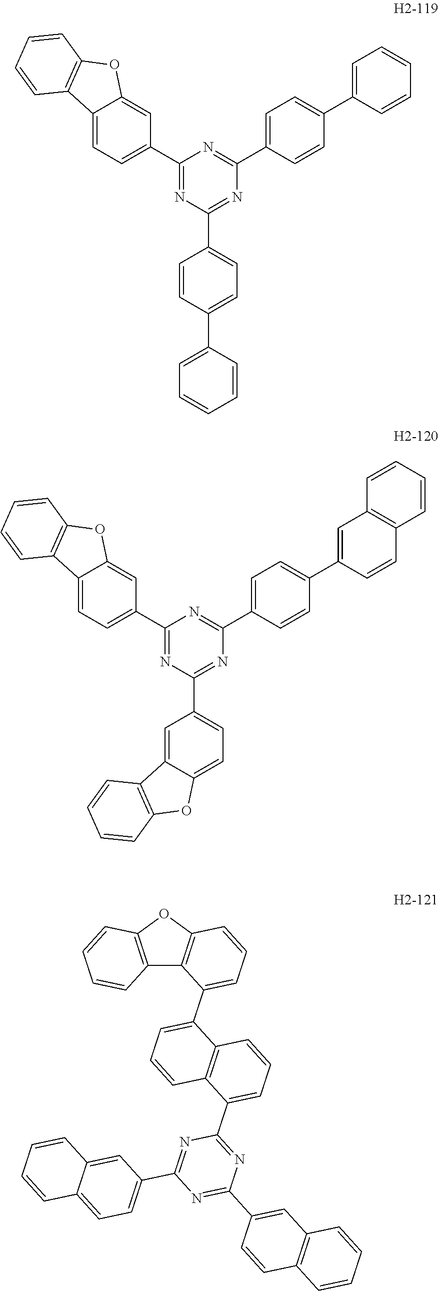 [SMILES: c1ccc(-c2ccc(-c3nc(-c4ccc(-c5ccccc5)cc4)nc(-c4ccc5c(c4)oc4ccccc45)n3)cc2)cc1 ; c1ccc2cc(-c3ccc(-c4nc(-c5ccc6c(c5)oc5ccccc56)nc(-c5ccc6oc7ccccc7c6c5)n4)cc3)ccc2c1 ; c1ccc2cc(-c3nc(-c4ccc5ccccc5c4)nc(-c4cccc5c(-c6cccc7oc8ccccc8c67)cccc45)n3)ccc2c1] 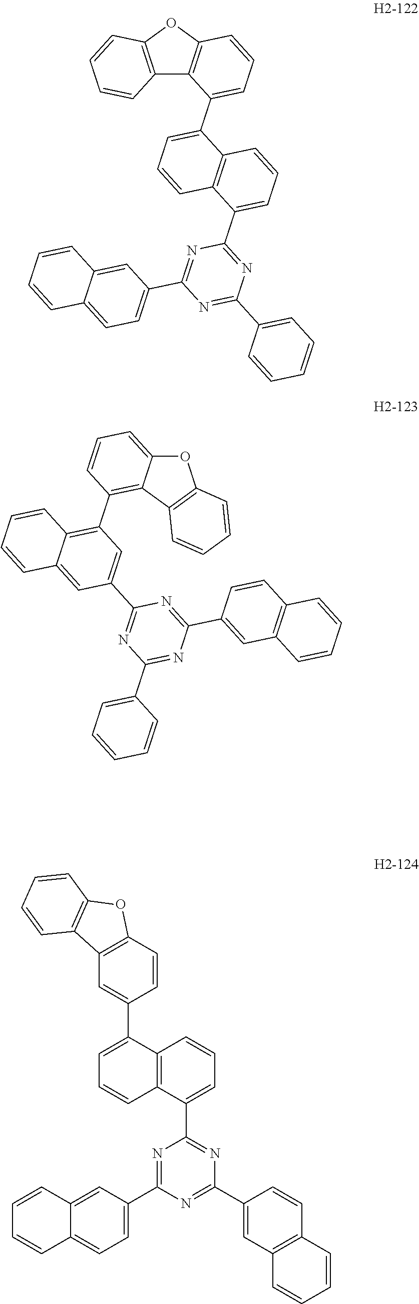 [SMILES: c1ccc(-c2nc(-c3ccc4ccccc4c3)nc(-c3cccc4c(-c5cccc6oc7ccccc7c56)cccc34)n2)cc1 ; c1ccc(-c2nc(-c3ccc4ccccc4c3)nc(-c3cc(-c4cccc5oc6ccccc6c45)c4ccccc4c3)n2)cc1 ; c1ccc2cc(-c3nc(-c4ccc5ccccc5c4)nc(-c4cccc5c(-c6ccc7oc8ccccc8c7c6)cccc45)n3)ccc2c1] 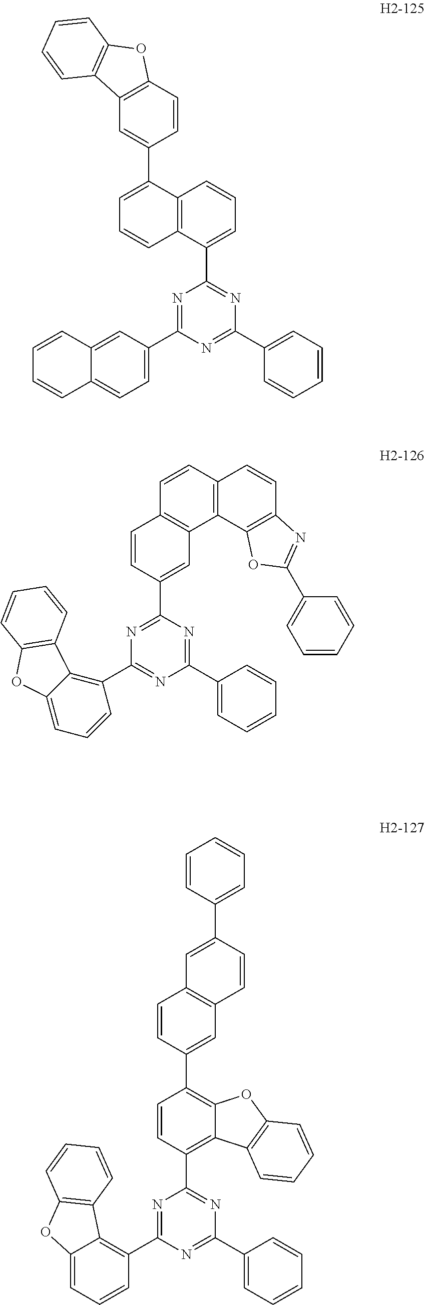 [SMILES: c1ccc(-c2nc(-c3ccc4ccccc4c3)nc(-c3cccc4c(-c5ccc6oc7ccccc7c6c5)cccc34)n2)cc1 ; c1ccc(-c2nc(-c3ccc4ccc5ccc6nc(-c7ccccc7)oc6c5c4c3)nc(-c3cccc4oc5ccccc5c34)n2)cc1 ; c1ccc(-c2ccc3cc(-c4ccc(-c5nc(-c6ccccc6)nc(-c6cccc7oc8ccccc8c67)n5)c5c4oc4ccccc45)ccc3c2)cc1] 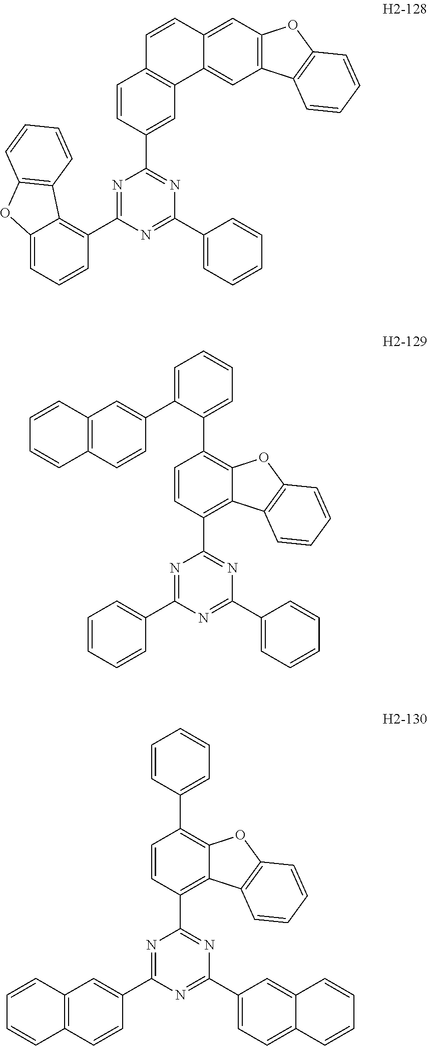 [SMILES: c1ccc(-c2nc(-c3ccc4ccc5cc6oc7ccccc7c6cc5c4c3)nc(-c3cccc4oc5ccccc5c34)n2)cc1 ; c1ccc(-c2nc(-c3ccccc3)nc(-c3ccc(-c4ccccc4-c4ccc5ccccc5c4)c4oc5ccccc5c34)n2)cc1 ; c1ccc(-c2ccc(-c3nc(-c4ccc5ccccc5c4)nc(-c4ccc5ccccc5c4)n3)c3c2oc2ccccc23)cc1] 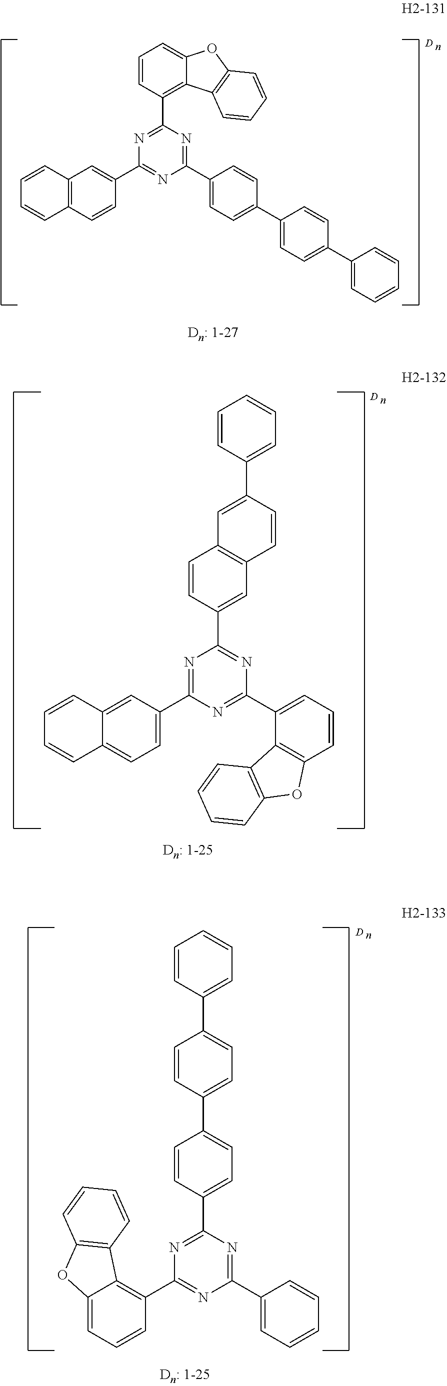 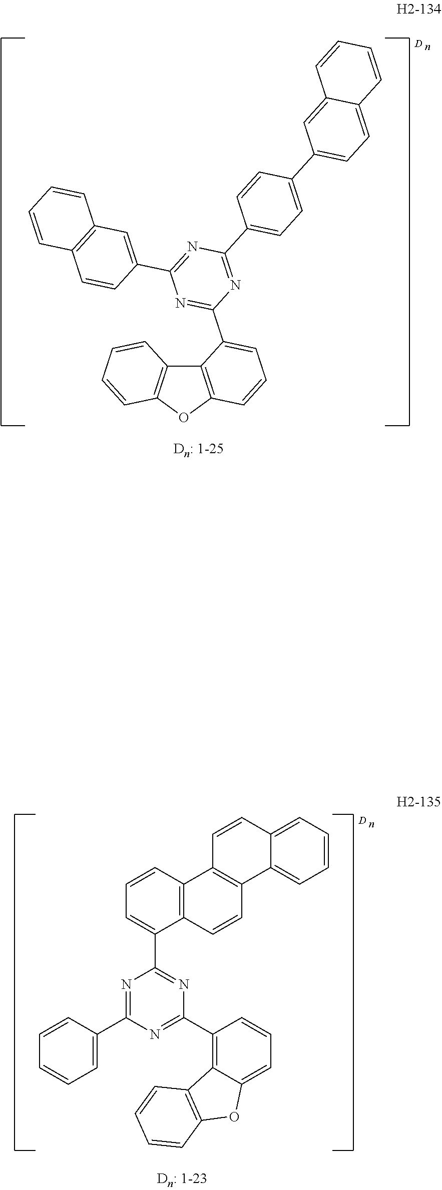 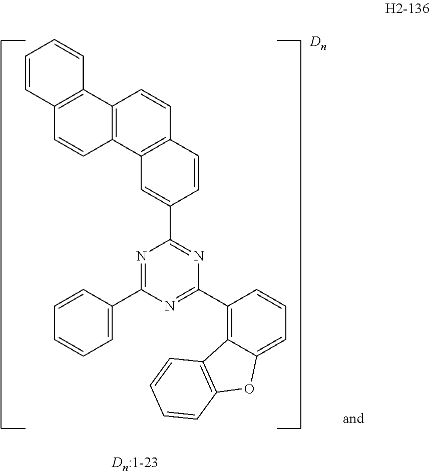
wherein in each of Compounds H2-131 to H2-136, Dₙ means that 1 to 23, 1 to 25, or 1 to 27 hydrogens are replaced with deuterium.

12. A plurality of host materials comprising a first host material comprising the compound according to claim 1, and a second host material comprising a compound represented by the following Formula 5: 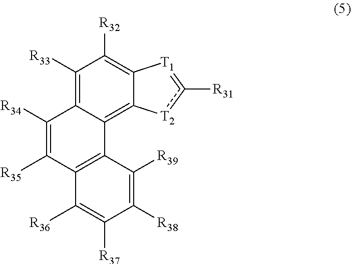
wherein in Formula 5,
T₁ and T₂, each independently, represent —N═, —NR₄₀—, —O—, or —S—, with the proviso that any one of T₁ and T₂ represents —N═, and the other of T₁ and T₂ represents —NR₄₀—, —O—, or —S—;
R₃₁ represents a substituted or unsubstituted (C6-C30)aryl, or a substituted or unsubstituted (3- to 30-membered)heteroaryl;
R₃₂ to R₄₀, each independently, are represented by the following Formula 5-1, or represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted mono- or di-(C6-C30)arylamino, a substituted or unsubstituted mono- or di-(3- to 30-membered)heteroarylamino, or a substituted or unsubstituted (C6-C30)aryl(3- to 30-membered)heteroarylamino; or may be linked to an adjacent substituent(s) to form a ring(s);
with the proviso that at least one of R₃₂ to R₃₉ is represented by the following Formula 5-1: 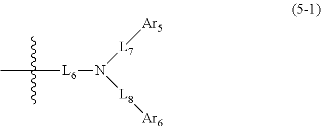
wherein in Formula 5-1,
L₆ to L₈, each independently, represent a single bond, a substituted or unsubstituted (C6-C30)arylene, or a substituted or unsubstituted (3- to 30-membered)heteroarylene; and
Ar₅ and Ar₆, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted (C6-C30)aryl(3- to 30-membered)heteroarylamino, or a substituted or unsubstituted fused ring group of a (C3-C30)aliphatic ring(s) and a (C6-C30)aromatic ring(s), or may be linked to an adjacent substituent(s) to form a ring(s).

13. The plurality of host materials according to claim 12, wherein the compound represented by Formula 5 is at least one selected from the following compounds: 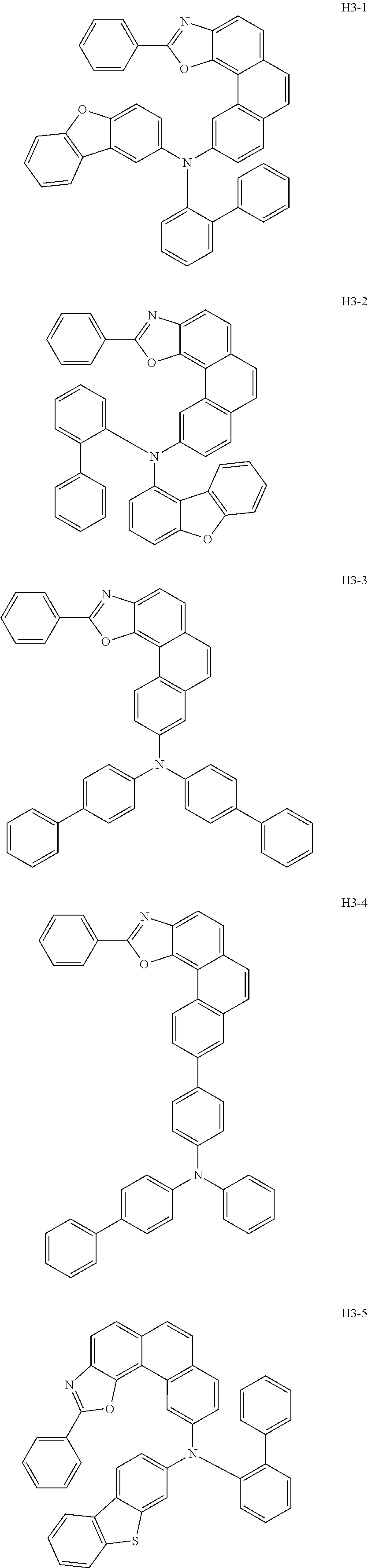 [SMILES: c1ccc(-c2nc3ccc4ccc5ccc(N(c6ccc7oc8ccccc8c7c6)c6ccccc6-c6ccccc6)cc5c4c3o2)cc1 ; c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1 ; c1ccc(-c2ccc(N(c3ccccc3)c3ccc(-c4ccc5c(ccc6ccc7nc(-c8ccccc8)oc7c65)c4)cc3)cc2)cc1 ; c1ccc(-c2nc3ccc4ccc5ccc(N(c6ccc7c(c6)sc6ccccc67)c6ccccc6-c6ccccc6)cc5c4c3o2)cc1] 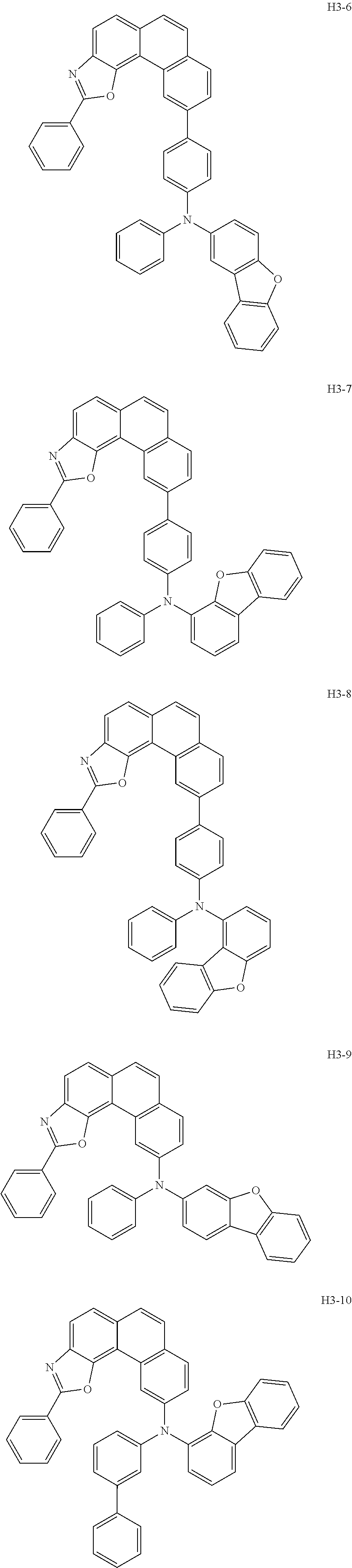 [SMILES: c1ccc(-c2nc3ccc4ccc5ccc(-c6ccc(N(c7ccccc7)c7ccc8oc9ccccc9c8c7)cc6)cc5c4c3o2)cc1 ; c1ccc(-c2nc3ccc4ccc5ccc(-c6ccc(N(c7ccccc7)c7cccc8c7oc7ccccc78)cc6)cc5c4c3o2)cc1 ; c1ccc(-c2nc3ccc4ccc5ccc(-c6ccc(N(c7ccccc7)c7cccc8oc9ccccc9c78)cc6)cc5c4c3o2)cc1 ; c1ccc(-c2nc3ccc4ccc5ccc(N(c6ccccc6)c6ccc7c(c6)oc6ccccc67)cc5c4c3o2)cc1 ; c1ccc(-c2cccc(N(c3ccc4ccc5ccc6nc(-c7ccccc7)oc6c5c4c3)c3cccc4c3oc3ccccc34)c2)cc1] 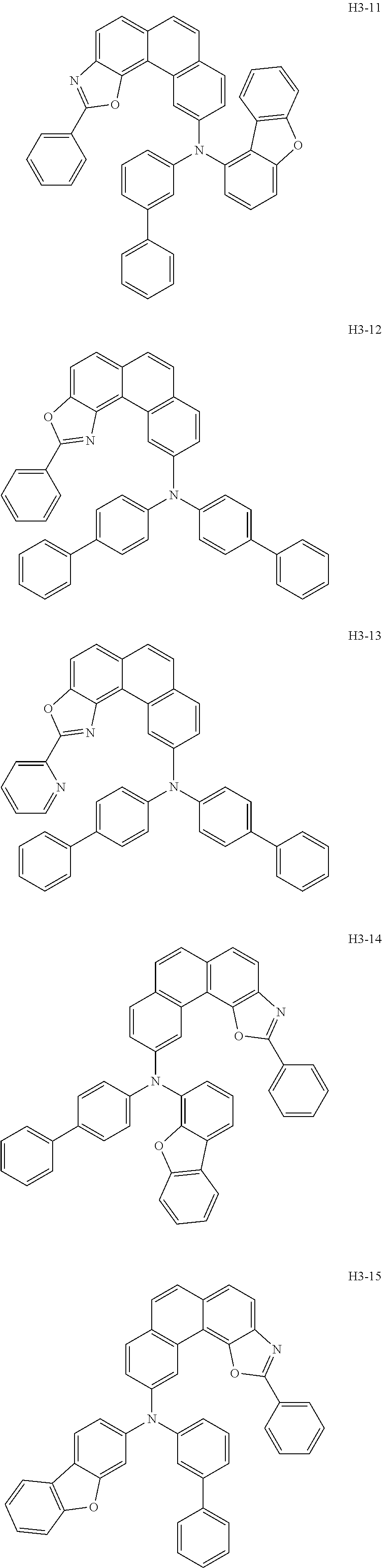 [SMILES: c1ccc(-c2cccc(N(c3ccc4ccc5ccc6nc(-c7ccccc7)oc6c5c4c3)c3cccc4oc5ccccc5c34)c2)cc1 ; c1ccc(-c2ccc(N(c3ccc(-c4ccccc4)cc3)c3ccc4ccc5ccc6oc(-c7ccccc7)nc6c5c4c3)cc2)cc1 ; c1ccc(-c2ccc(N(c3ccc(-c4ccccc4)cc3)c3ccc4ccc5ccc6oc(-c7ccccn7)nc6c5c4c3)cc2)cc1 ; c1ccc(-c2ccc(N(c3ccc4ccc5ccc6nc(-c7ccccc7)oc6c5c4c3)c3cccc4c3oc3ccccc34)cc2)cc1 ; c1ccc(-c2cccc(N(c3ccc4c(c3)oc3ccccc34)c3ccc4ccc5ccc6nc(-c7ccccc7)oc6c5c4c3)c2)cc1] 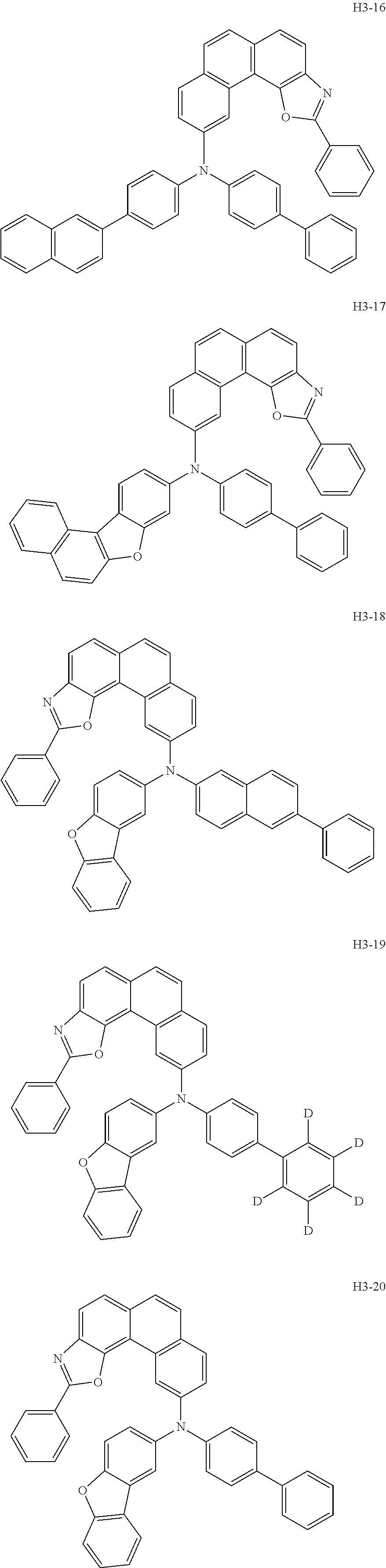 [SMILES: c1ccc(-c2ccc(N(c3ccc(-c4ccc5ccccc5c4)cc3)c3ccc4ccc5ccc6nc(-c7ccccc7)oc6c5c4c3)cc2)cc1 ; c1ccc(-c2ccc(N(c3ccc4c(c3)oc3ccc5ccccc5c34)c3ccc4ccc5ccc6nc(-c7ccccc7)oc6c5c4c3)cc2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1 ; [2H]c1c([2H])c([2H])c(-c2ccc(N(c3ccc4oc5ccccc5c4c3)c3ccc4ccc5ccc6nc(-c7ccccc7)oc6c5c4c3)cc2)c([2H])c1[2H] ; c1ccc(-c2ccc(N(c3ccc4oc5ccccc5c4c3)c3ccc4ccc5ccc6nc(-c7ccccc7)oc6c5c4c3)cc2)cc1] 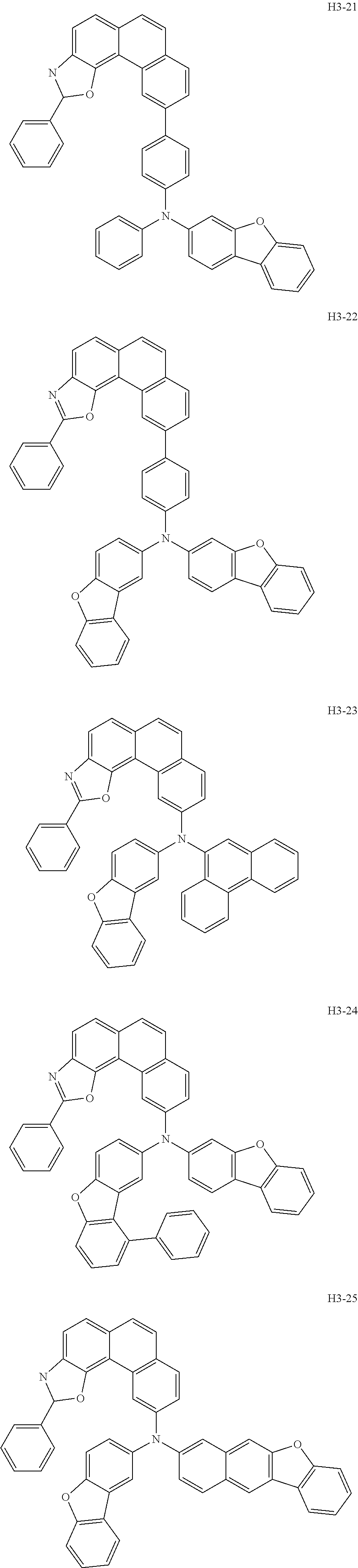 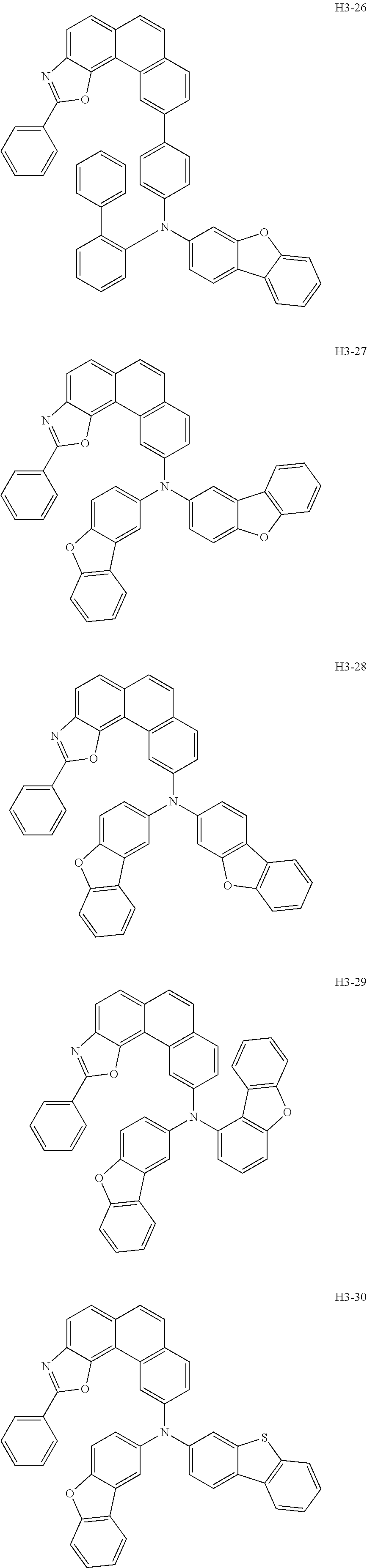 [SMILES: c1ccc(-c2nc3ccc4ccc5ccc(-c6ccc(N(c7ccc8c(c7)oc7ccccc78)c7ccccc7-c7ccccc7)cc6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3o2)cc1 ; c1ccc(-c2nc3ccc4ccc5ccc(N(c6ccc7oc8ccccc8c7c6)c6cccc7oc8ccccc8c67)cc5c4c3o2)cc1 ; c1ccc(-c2nc3ccc4ccc5ccc(N(c6ccc7c(c6)sc6ccccc67)c6ccc7oc8ccccc8c7c6)cc5c4c3o2)cc1] 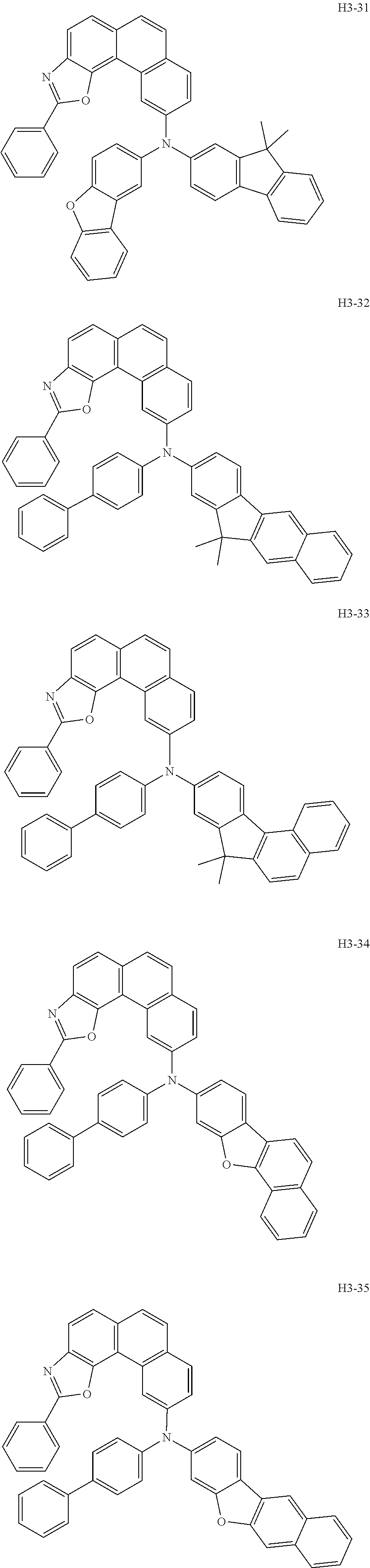 [SMILES: CC1(C)c2ccccc2-c2ccc(N(c3ccc4oc5ccccc5c4c3)c3ccc4ccc5ccc6nc(-c7ccccc7)oc6c5c4c3)cc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21 ; CC1(C)c2cc(N(c3ccc(-c4ccccc4)cc3)c3ccc4ccc5ccc6nc(-c7ccccc7)oc6c5c4c3)ccc2-c2c1ccc1ccccc21 ; c1ccc(-c2ccc(N(c3ccc4c(c3)oc3c5ccccc5ccc43)c3ccc4ccc5ccc6nc(-c7ccccc7)oc6c5c4c3)cc2)cc1 ; c1ccc(-c2ccc(N(c3ccc4c(c3)oc3cc5ccccc5cc34)c3ccc4ccc5ccc6nc(-c7ccccc7)oc6c5c4c3)cc2)cc1] 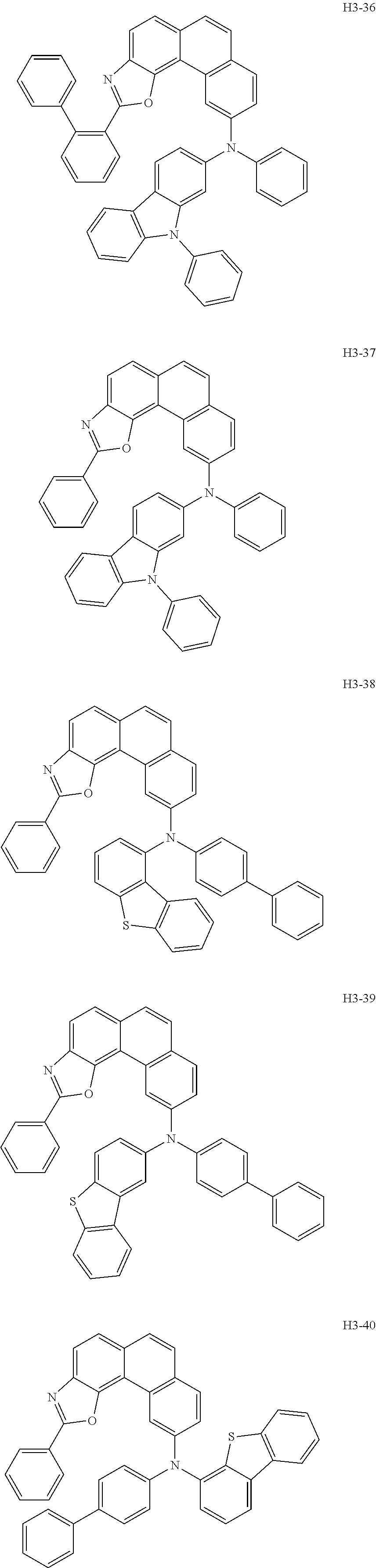 [SMILES: c1ccc(-c2ccccc2-c2nc3ccc4ccc5ccc(N(c6ccccc6)c6ccc7c8ccccc8n(-c8ccccc8)c7c6)cc5c4c3o2)cc1 ; c1ccc(-c2nc3ccc4ccc5ccc(N(c6ccccc6)c6ccc7c8ccccc8n(-c8ccccc8)c7c6)cc5c4c3o2)cc1 ; c1ccc(-c2ccc(N(c3ccc4ccc5ccc6nc(-c7ccccc7)oc6c5c4c3)c3cccc4sc5ccccc5c34)cc2)cc1 ; c1ccc(-c2ccc(N(c3ccc4sc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4c3sc3ccccc34)cc2)cc1] 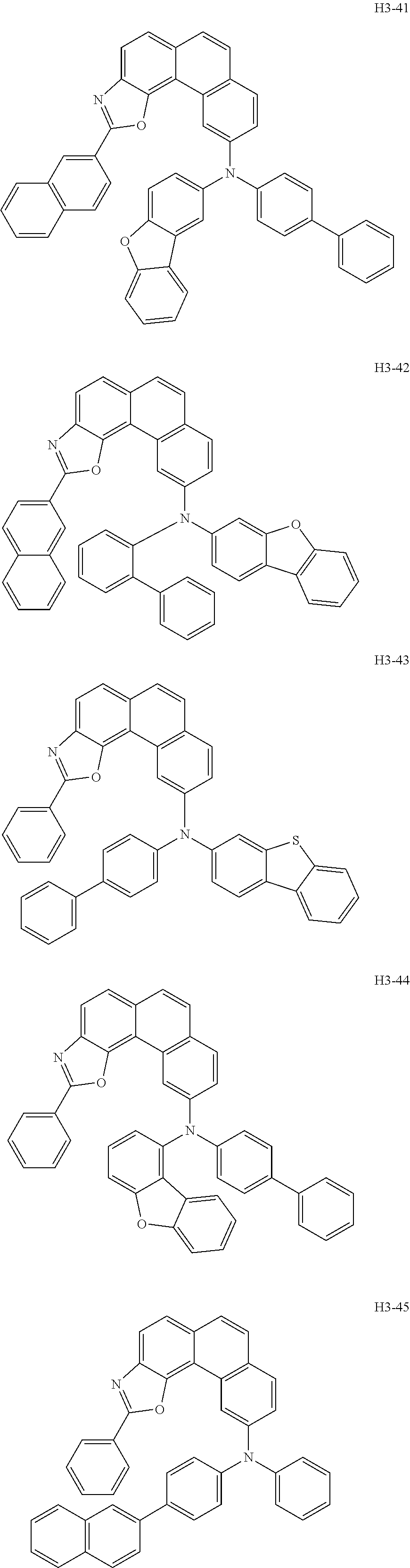 [SMILES: c1ccc(-c2ccc(N(c3ccc4oc5ccccc5c4c3)c3ccc4ccc5ccc6nc(-c7ccc8ccccc8c7)oc6c5c4c3)cc2)cc1 ; c1ccc(-c2ccccc2N(c2ccc3c(c2)oc2ccccc23)c2ccc3ccc4ccc5nc(-c6ccc7ccccc7c6)oc5c4c3c2)cc1 ; c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4oc5ccccc5c34)cc2)cc1 ; c1ccc(-c2nc3ccc4ccc5ccc(N(c6ccccc6)c6ccc(-c7ccc8ccccc8c7)cc6)cc5c4c3o2)cc1] 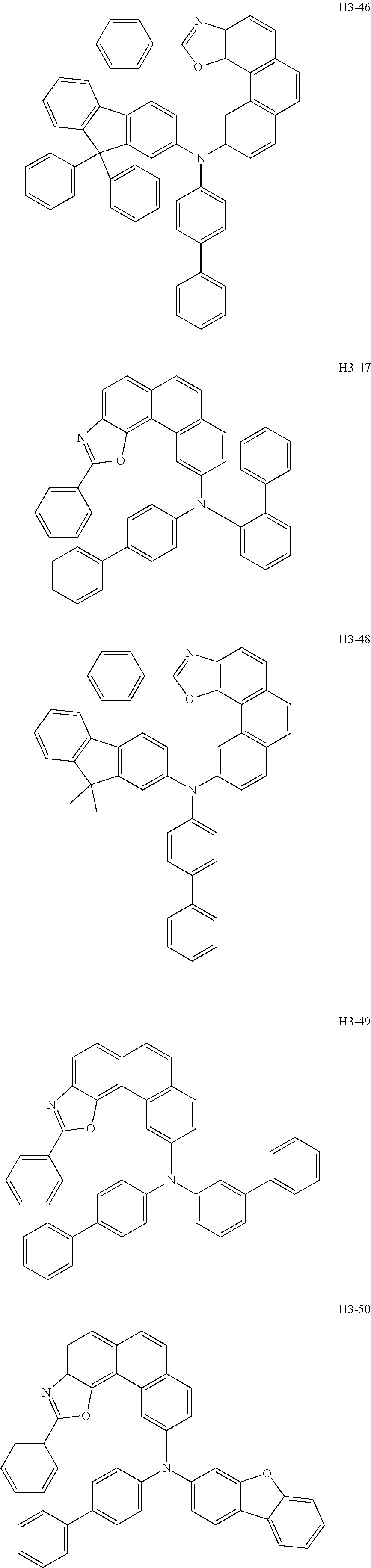 [SMILES: c1ccc(-c2ccc(N(c3ccc4c(c3)C(c3ccccc3)(c3ccccc3)c3ccccc3-4)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3ccccc3-c3ccccc3)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3cccc(-c4ccccc4)c3)c3ccc4ccc5ccc6nc(-c7ccccc7)oc6c5c4c3)cc2)cc1 ; c1ccc(-c2ccc(N(c3ccc4c(c3)oc3ccccc34)c3ccc4ccc5ccc6nc(-c7ccccc7)oc6c5c4c3)cc2)cc1] 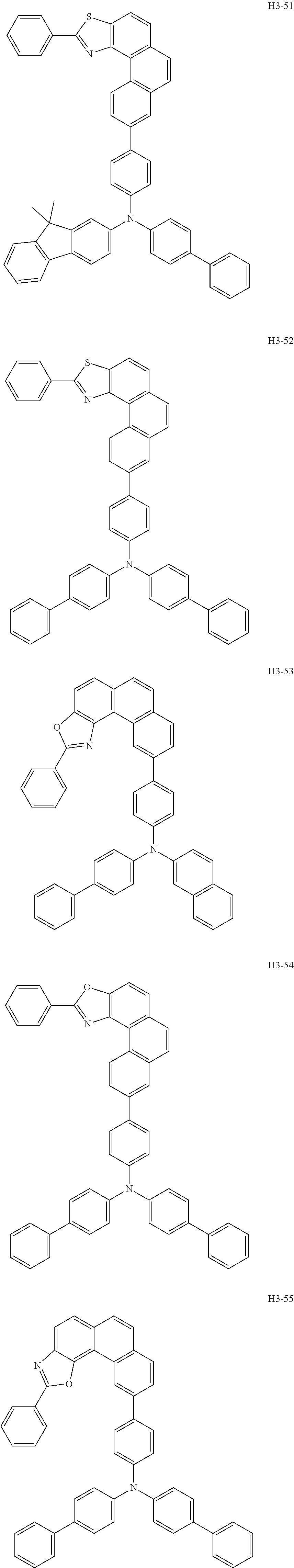 [SMILES: CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccccc4)cc3)c3ccc(-c4ccc5ccc6ccc7nc(-c8ccccc8)oc7c6c5c4)cc3)cc2)cc1] 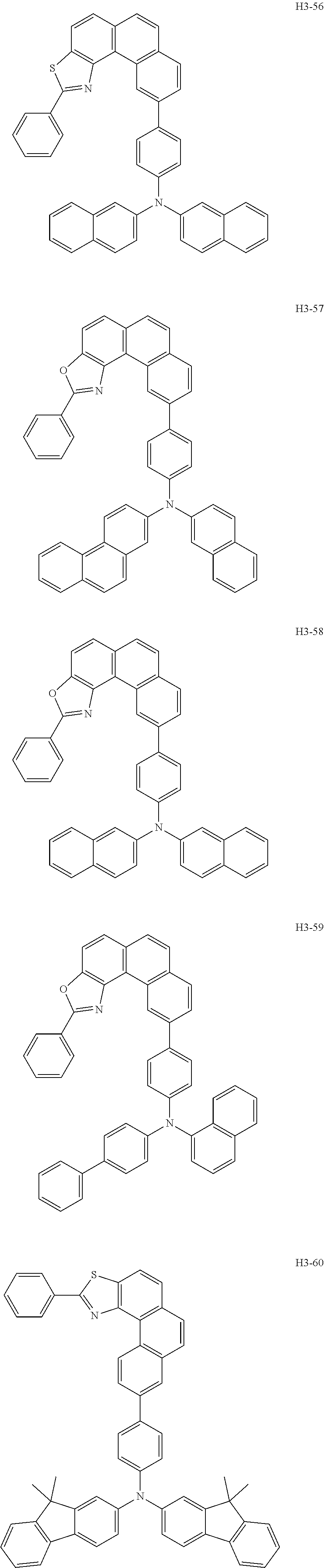 [SMILES: c1ccc(-c2nc3c(ccc4ccc5ccc(-c6ccc(N(c7ccc8ccccc8c7)c7ccc8ccccc8c7)cc6)cc5c43)s2)cc1 ; c1ccc(-c2nc3c(ccc4ccc5ccc(-c6ccc(N(c7ccc8ccccc8c7)c7ccc8c(ccc9ccccc98)c7)cc6)cc5c43)o2)cc1 ; c1ccc(-c2nc3c(ccc4ccc5ccc(-c6ccc(N(c7ccc8ccccc8c7)c7ccc8ccccc8c7)cc6)cc5c43)o2)cc1 ; c1ccc(-c2ccc(N(c3ccc(-c4ccc5ccc6ccc7oc(-c8ccccc8)nc7c6c5c4)cc3)c3cccc4ccccc34)cc2)cc1 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21] 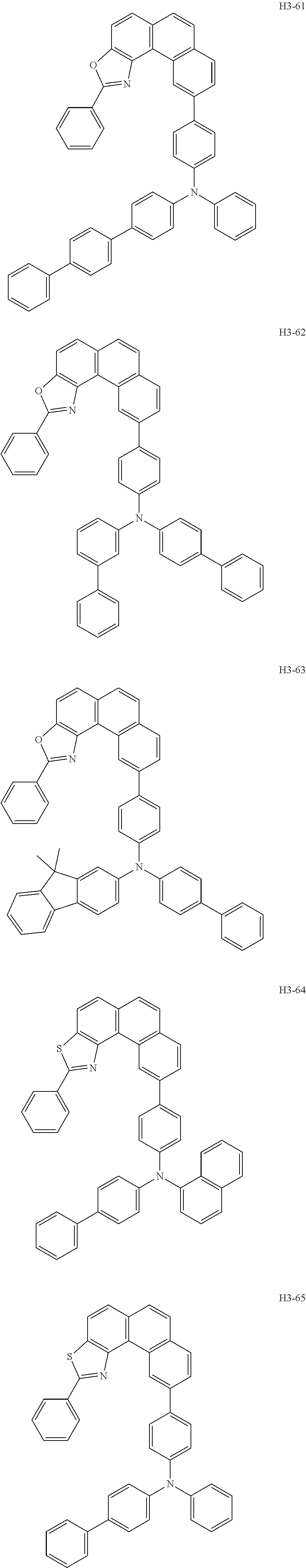 [SMILES: c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7sc(-c8ccccc8)nc7c6c5c4)cc3)c3cccc4ccccc34)cc2)cc1 ; c1ccc(-c2ccc(N(c3ccccc3)c3ccc(-c4ccc5ccc6ccc7sc(-c8ccccc8)nc7c6c5c4)cc3)cc2)cc1] 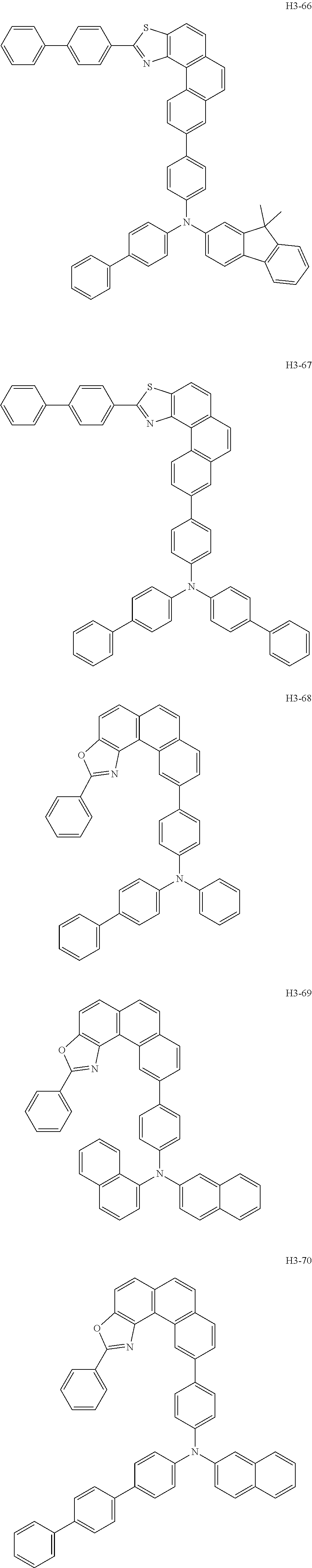 [SMILES: CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; c1ccc(-c2ccc(N(c3ccccc3)c3ccc(-c4ccc5ccc6ccc7oc(-c8ccccc8)nc7c6c5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1] 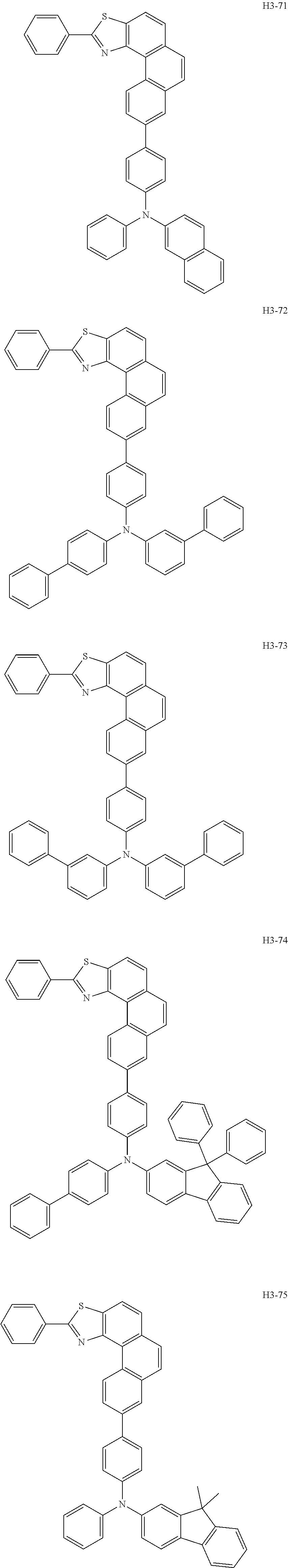 [SMILES: c1ccc(-c2nc3c(ccc4ccc5cc(-c6ccc(N(c7ccccc7)c7ccc8ccccc8c7)cc6)ccc5c43)s2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; CC1(C)c2ccccc2-c2ccc(N(c3ccccc3)c3ccc(-c4ccc5c(ccc6ccc7sc(-c8ccccc8)nc7c65)c4)cc3)cc21] 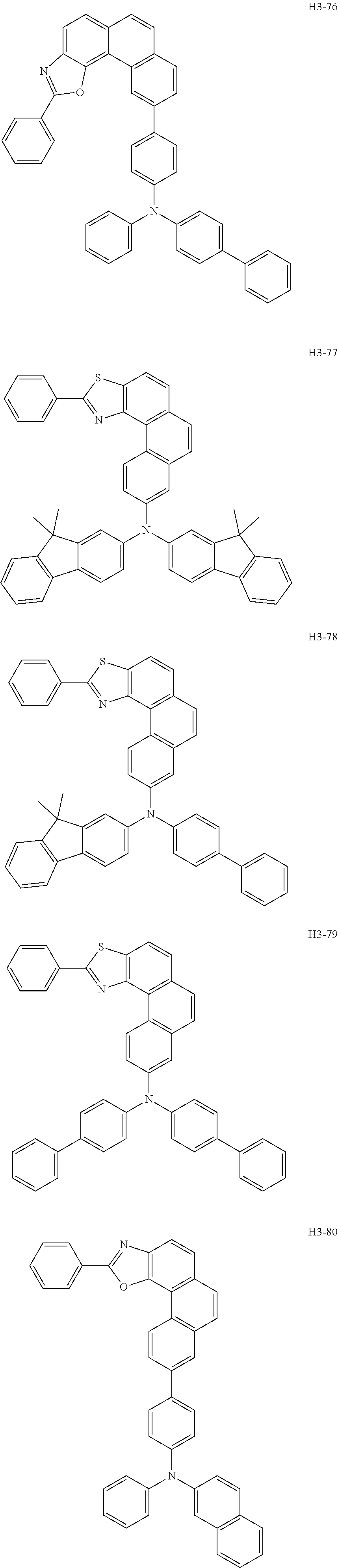 [SMILES: c1ccc(-c2ccc(N(c3ccccc3)c3ccc(-c4ccc5ccc6ccc7nc(-c8ccccc8)oc7c6c5c4)cc3)cc2)cc1 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; CC1(C)c2ccccc2-c2ccc(N(c3ccc(-c4ccccc4)cc3)c3ccc4c(ccc5ccc6sc(-c7ccccc7)nc6c54)c3)cc21 ; c1ccc(-c2ccc(N(c3ccc(-c4ccccc4)cc3)c3ccc4c(ccc5ccc6sc(-c7ccccc7)nc6c54)c3)cc2)cc1 ; c1ccc(-c2nc3ccc4ccc5cc(-c6ccc(N(c7ccccc7)c7ccc8ccccc8c7)cc6)ccc5c4c3o2)cc1] 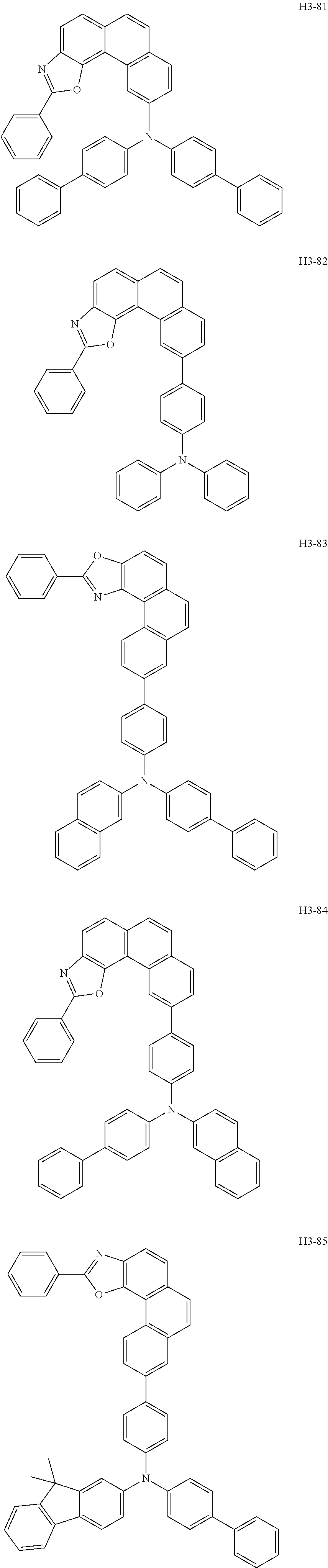 [SMILES: c1ccc(-c2ccc(N(c3ccc(-c4ccccc4)cc3)c3ccc4ccc5ccc6nc(-c7ccccc7)oc6c5c4c3)cc2)cc1 ; c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1 ; c1ccc(-c2ccc(N(c3ccc(-c4ccc5ccc6ccc7nc(-c8ccccc8)oc7c6c5c4)cc3)c3ccc4ccccc4c3)cc2)cc1 ; CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21] 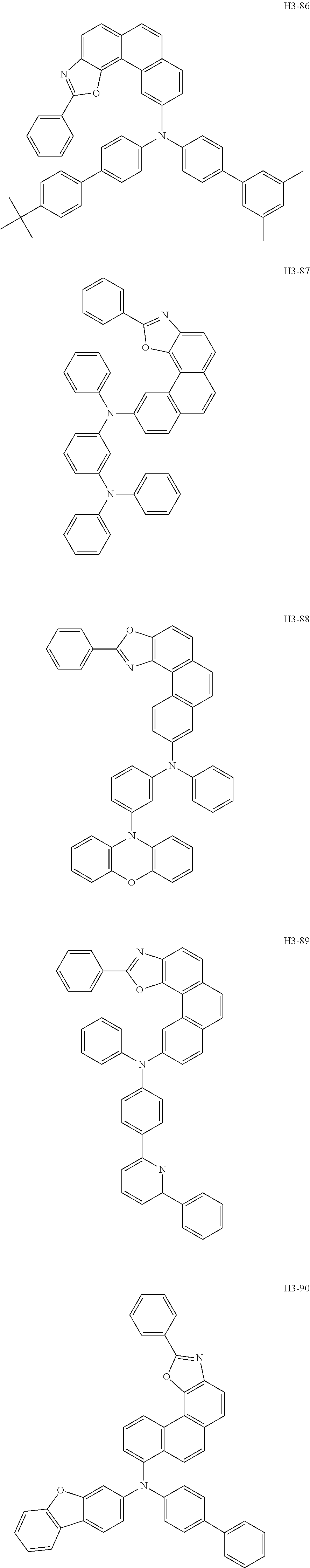 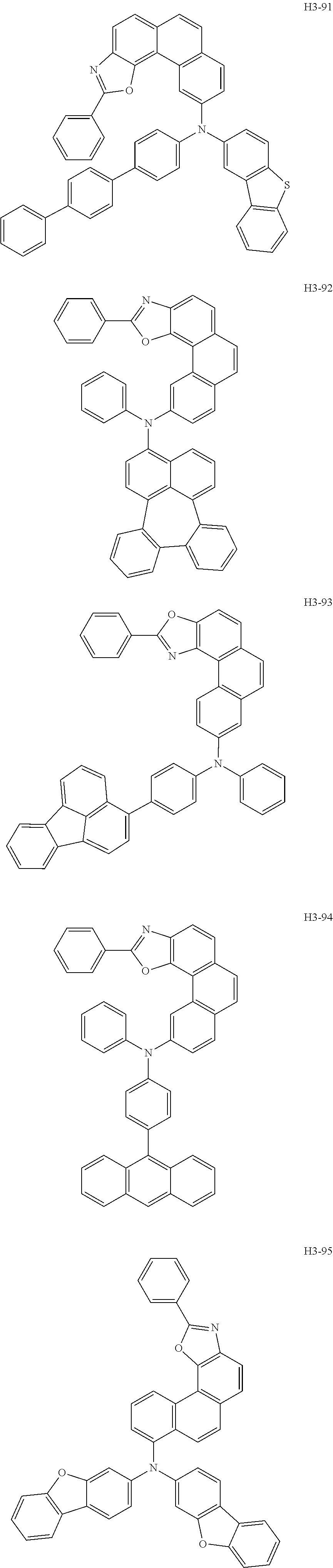 [SMILES: c1ccc(-c2ccc(-c3ccc(N(c4ccc5sc6ccccc6c5c4)c4ccc5ccc6ccc7nc(-c8ccccc8)oc7c6c5c4)cc3)cc2)cc1 ; c1ccc(-c2nc3ccc4ccc5ccc(N(c6ccccc6)c6ccc7c8c(cccc68)-c6ccccc6-c6ccccc6-7)cc5c4c3o2)cc1 ; c1ccc(-c2nc3c(ccc4ccc5cc(N(c6ccccc6)c6ccc(-c7ccc8c9c(cccc79)-c7ccccc7-8)cc6)ccc5c43)o2)cc1 ; c1ccc(-c2nc3ccc4ccc5ccc(N(c6ccccc6)c6ccc(-c7c8ccccc8cc8ccccc78)cc6)cc5c4c3o2)cc1 ; c1ccc(-c2nc3ccc4ccc5c(N(c6ccc7c(c6)oc6ccccc67)c6ccc7c(c6)oc6ccccc67)cccc5c4c3o2)cc1] 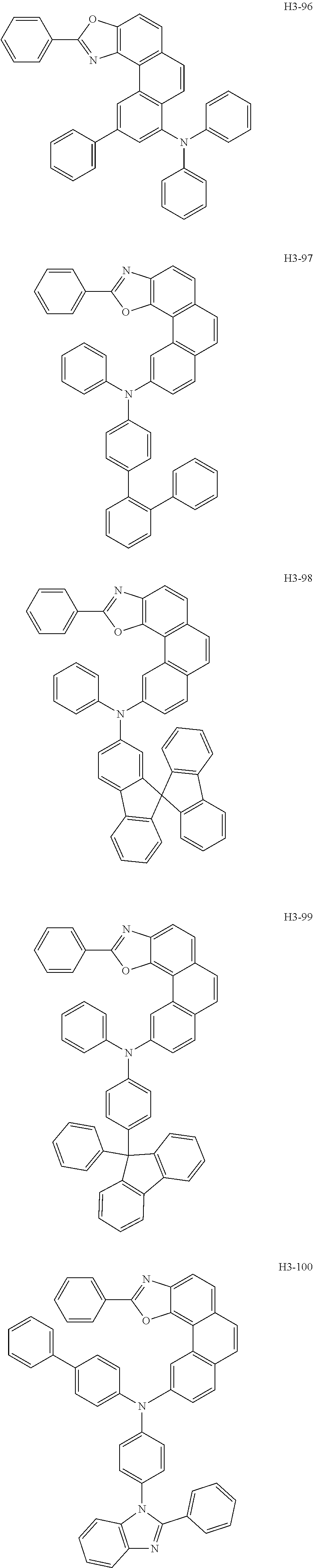 [SMILES: c1ccc(-c2cc(N(c3ccccc3)c3ccccc3)c3ccc4ccc5oc(-c6ccccc6)nc5c4c3c2)cc1 ; c1ccc(-c2nc3ccc4ccc5ccc(N(c6ccccc6)c6ccc(-c7ccccc7-c7ccccc7)cc6)cc5c4c3o2)cc1 ; c1ccc(-c2nc3ccc4ccc5ccc(N(c6ccccc6)c6ccc7c(c6)C6(c8ccccc8-c8ccccc86)c6ccccc6-7)cc5c4c3o2)cc1 ; c1ccc(-c2nc3ccc4ccc5ccc(N(c6ccccc6)c6ccc(C7(c8ccccc8)c8ccccc8-c8ccccc87)cc6)cc5c4c3o2)cc1 ; c1ccc(-c2ccc(N(c3ccc(-n4c(-c5ccccc5)nc5ccccc54)cc3)c3ccc4ccc5ccc6nc(-c7ccccc7)oc6c5c4c3)cc2)cc1] 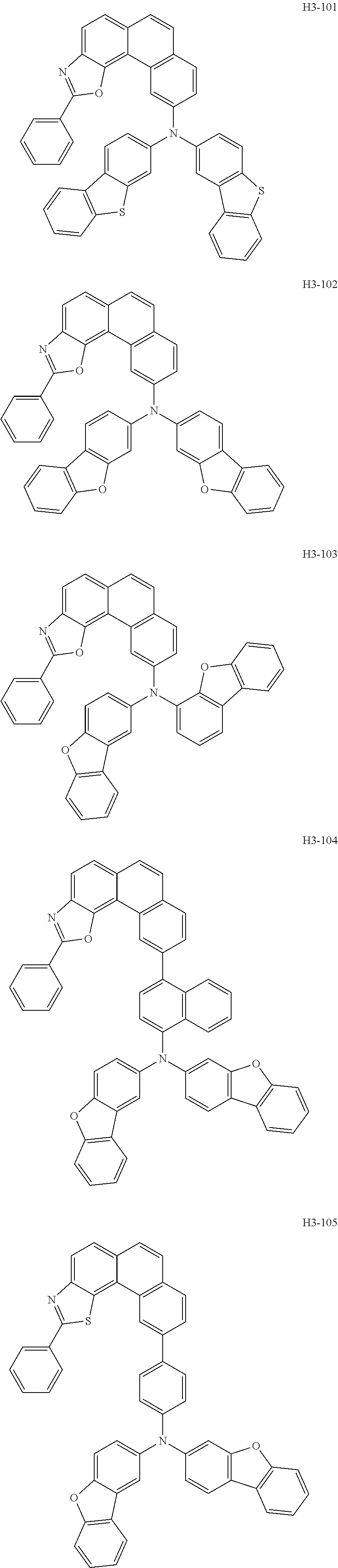 [SMILES: c1ccc(-c2nc3ccc4ccc5ccc(N(c6ccc7c(c6)sc6ccccc67)c6ccc7sc8ccccc8c7c6)cc5c4c3o2)cc1 ; c1ccc(-c2nc3ccc4ccc5ccc(N(c6ccc7c(c6)oc6ccccc67)c6ccc7c(c6)oc6ccccc67)cc5c4c3o2)cc1 ; c1ccc(-c2nc3ccc4ccc5ccc(N(c6ccc7oc8ccccc8c7c6)c6cccc7c6oc6ccccc67)cc5c4c3o2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3o2)cc1 ; c1ccc(-c2nc3ccc4ccc5ccc(-c6ccc(N(c7ccc8c(c7)oc7ccccc78)c7ccc8oc9ccccc9c8c7)cc6)cc5c4c3s2)cc1] 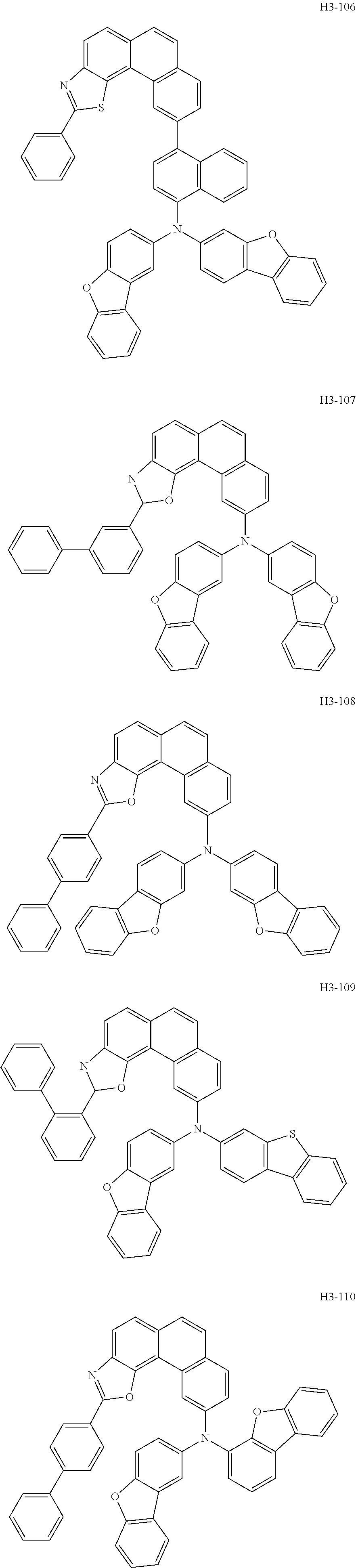 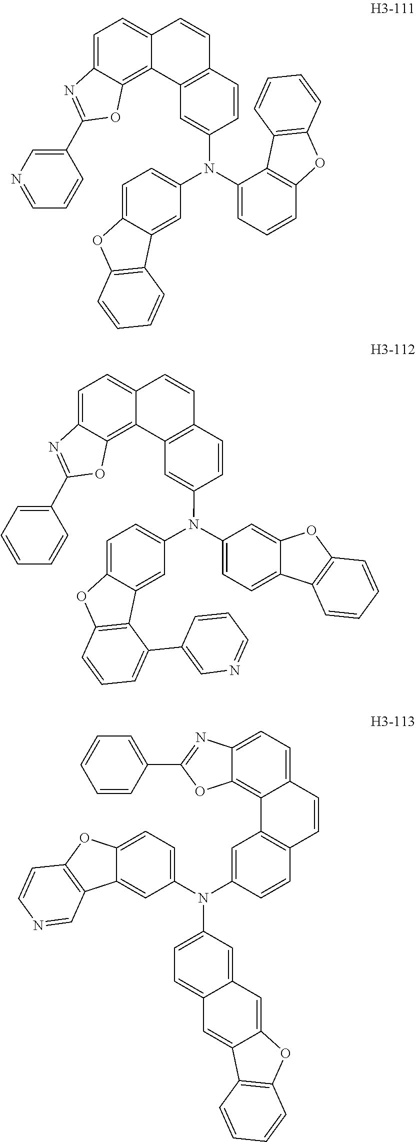 [SMILES: c1cncc(-c2nc3ccc4ccc5ccc(N(c6ccc7oc8ccccc8c7c6)c6cccc7oc8ccccc8c67)cc5c4c3o2)c1 ; c1ccc(-c2nc3ccc4ccc5ccc(N(c6ccc7c(c6)oc6ccccc67)c6ccc7oc8cccc(-c9cccnc9)c8c7c6)cc5c4c3o2)cc1 ; c1ccc(-c2nc3ccc4ccc5ccc(N(c6ccc7cc8c(cc7c6)oc6ccccc68)c6ccc7oc8ccncc8c7c6)cc5c4c3o2)cc1] 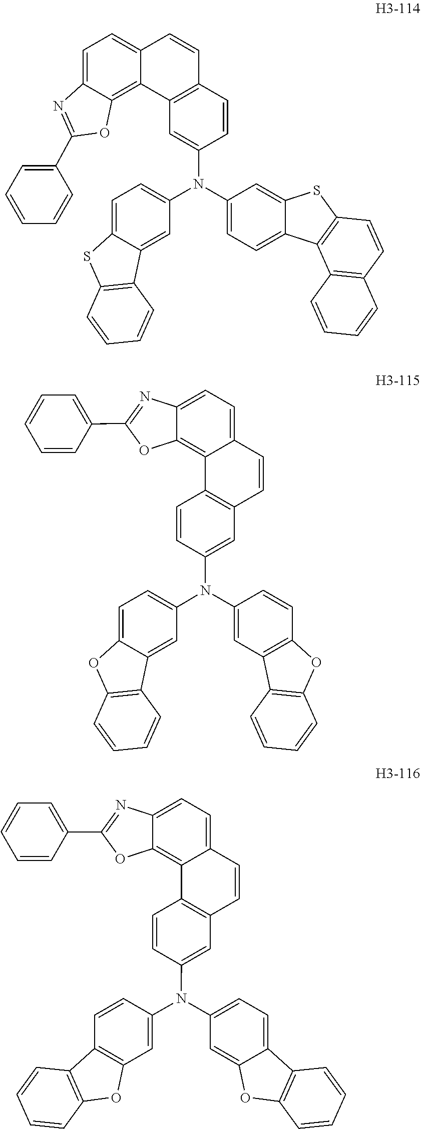 [SMILES: c1ccc(-c2nc3ccc4ccc5ccc(N(c6ccc7c(c6)sc6ccc8ccccc8c67)c6ccc7sc8ccccc8c7c6)cc5c4c3o2)cc1 ; c1ccc(-c2nc3ccc4ccc5cc(N(c6ccc7oc8ccccc8c7c6)c6ccc7oc8ccccc8c7c6)ccc5c4c3o2)cc1 ; c1ccc(-c2nc3ccc4ccc5cc(N(c6ccc7c(c6)oc6ccccc67)c6ccc7c(c6)oc6ccccc67)ccc5c4c3o2)cc1] 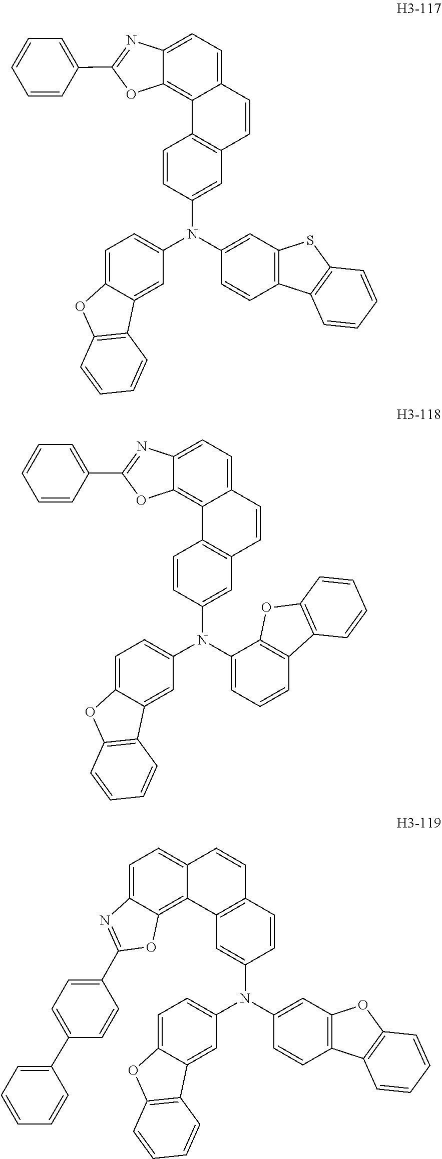 [SMILES: c1ccc(-c2nc3ccc4ccc5cc(N(c6ccc7c(c6)sc6ccccc67)c6ccc7oc8ccccc8c7c6)ccc5c4c3o2)cc1 ; c1ccc(-c2nc3ccc4ccc5cc(N(c6ccc7oc8ccccc8c7c6)c6cccc7c6oc6ccccc67)ccc5c4c3o2)cc1 ; c1ccc(-c2ccc(-c3nc4ccc5ccc6ccc(N(c7ccc8c(c7)oc7ccccc78)c7ccc8oc9ccccc9c8c7)cc6c5c4o3)cc2)cc1] 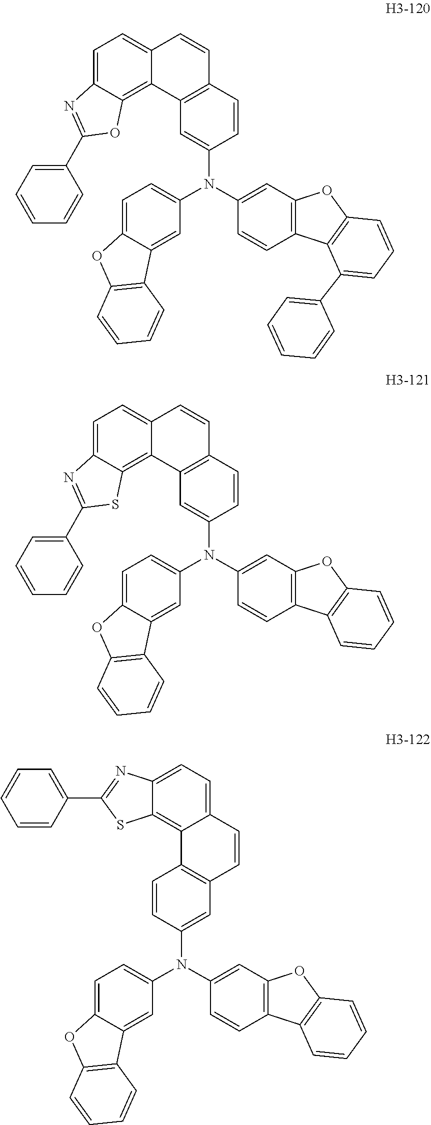 [SMILES: c1ccc(-c2nc3ccc4ccc5ccc(N(c6ccc7c(c6)oc6cccc(-c8ccccc8)c67)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3s2)cc1 ; c1ccc(-c2nc3ccc4ccc5cc(N(c6ccc7c(c6)oc6ccccc67)c6ccc7oc8ccccc8c7c6)ccc5c4c3s2)cc1] 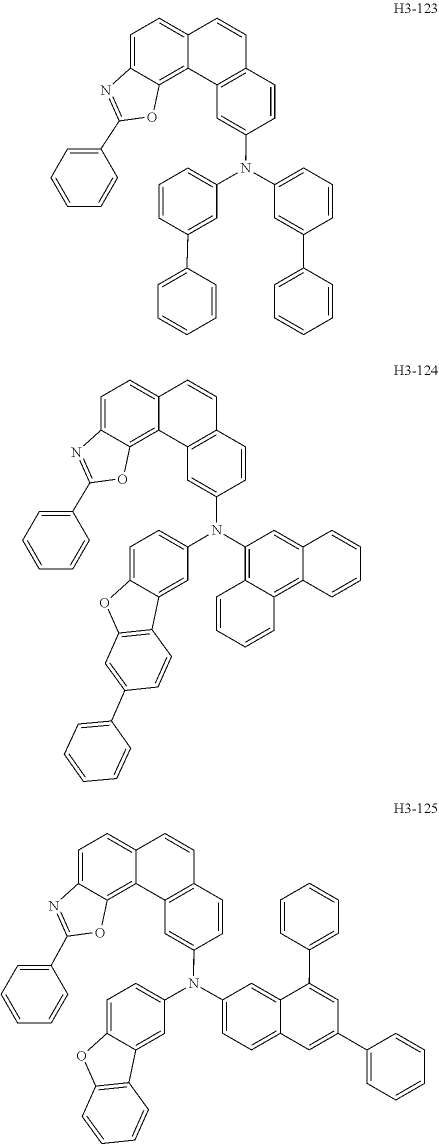 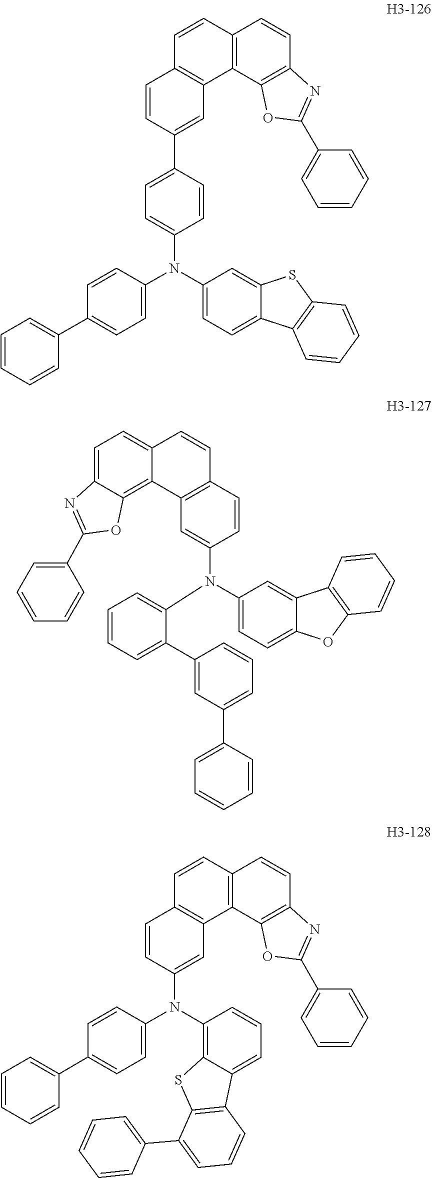 [SMILES: c1ccc(-c2ccc(N(c3ccc(-c4ccc5ccc6ccc7nc(-c8ccccc8)oc7c6c5c4)cc3)c3ccc4c(c3)sc3ccccc34)cc2)cc1 ; c1ccc(-c2cccc(-c3ccccc3N(c3ccc4oc5ccccc5c4c3)c3ccc4ccc5ccc6nc(-c7ccccc7)oc6c5c4c3)c2)cc1 ; c1ccc(-c2ccc(N(c3ccc4ccc5ccc6nc(-c7ccccc7)oc6c5c4c3)c3cccc4c3sc3c(-c5ccccc5)cccc34)cc2)cc1] 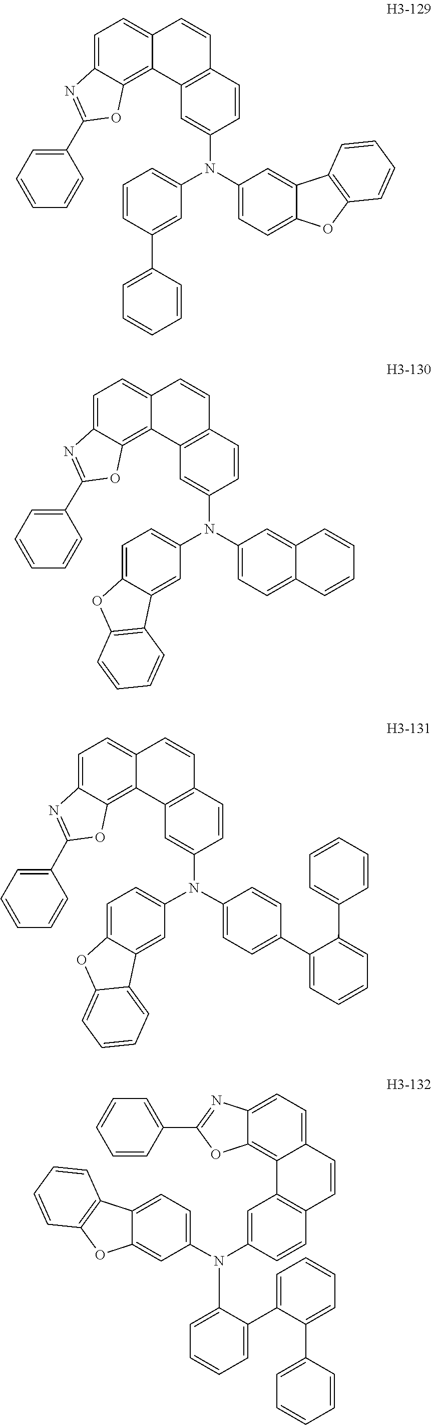 [SMILES: c1ccc(-c2cccc(N(c3ccc4oc5ccccc5c4c3)c3ccc4ccc5ccc6nc(-c7ccccc7)oc6c5c4c3)c2)cc1 ; c1ccc(-c2nc3ccc4ccc5ccc(N(c6ccc7ccccc7c6)c6ccc7oc8ccccc8c7c6)cc5c4c3o2)cc1 ; c1ccc(-c2nc3ccc4ccc5ccc(N(c6ccc(-c7ccccc7-c7ccccc7)cc6)c6ccc7oc8ccccc8c7c6)cc5c4c3o2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1] 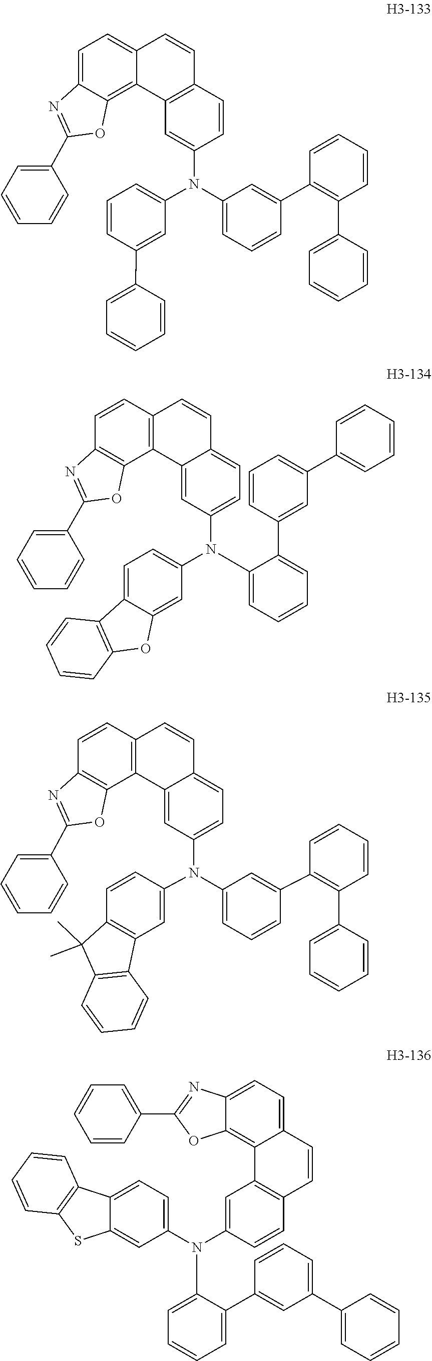 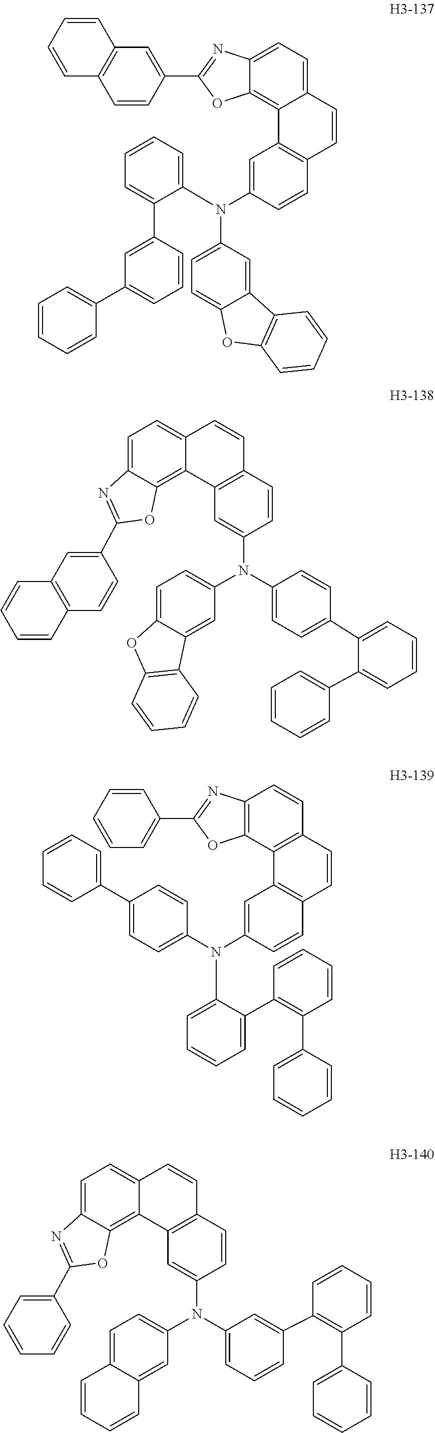 [SMILES: c1ccc(-c2cccc(-c3ccccc3N(c3ccc4oc5ccccc5c4c3)c3ccc4ccc5ccc6nc(-c7ccc8ccccc8c7)oc6c5c4c3)c2)cc1 ; c1ccc(-c2ccccc2-c2ccc(N(c3ccc4oc5ccccc5c4c3)c3ccc4ccc5ccc6nc(-c7ccc8ccccc8c7)oc6c5c4c3)cc2)cc1 ; c1ccc(-c2ccc(N(c3ccc4ccc5ccc6nc(-c7ccccc7)oc6c5c4c3)c3ccccc3-c3ccccc3-c3ccccc3)cc2)cc1 ; c1ccc(-c2nc3ccc4ccc5ccc(N(c6cccc(-c7ccccc7-c7ccccc7)c6)c6ccc7ccccc7c6)cc5c4c3o2)cc1] 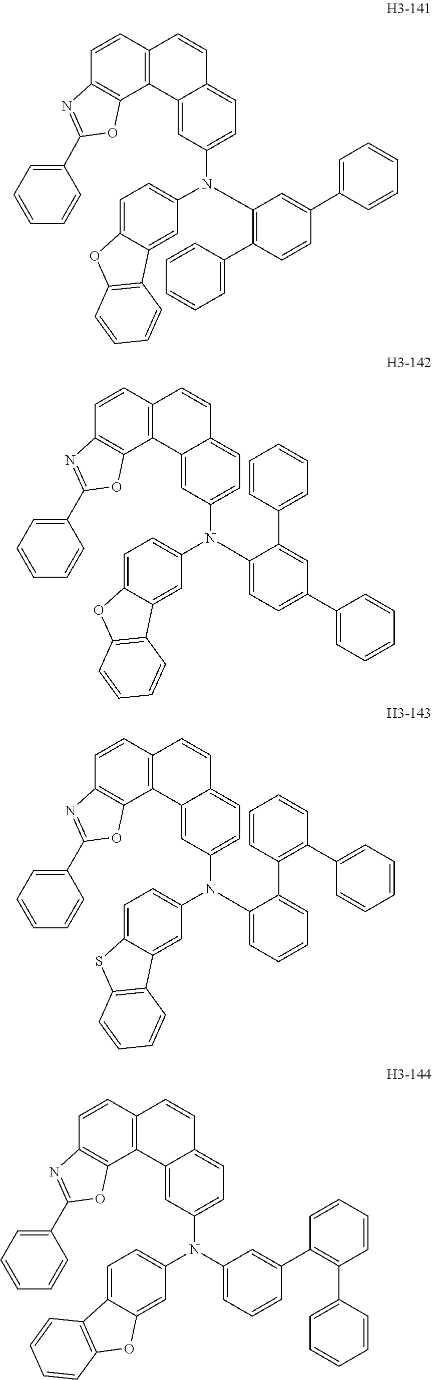 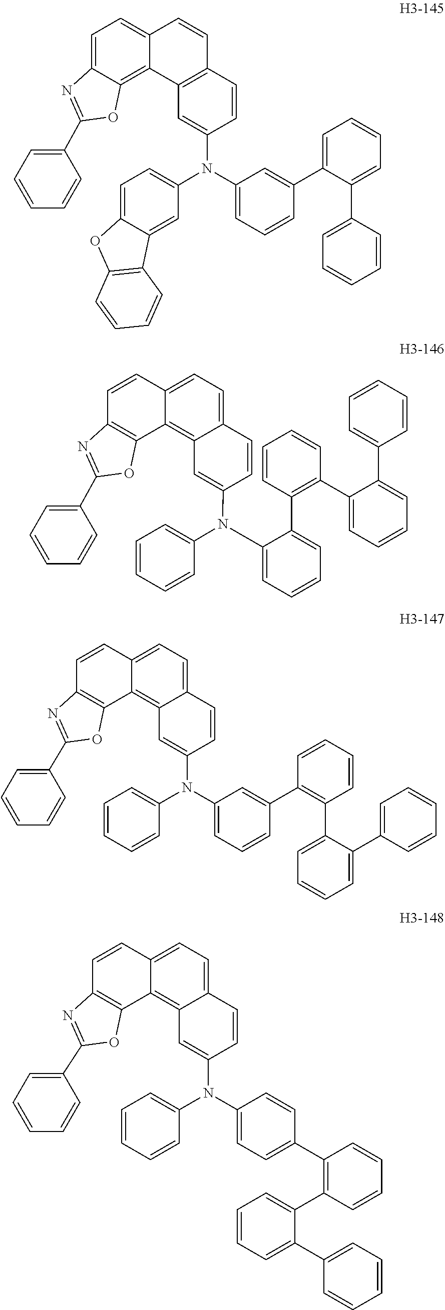 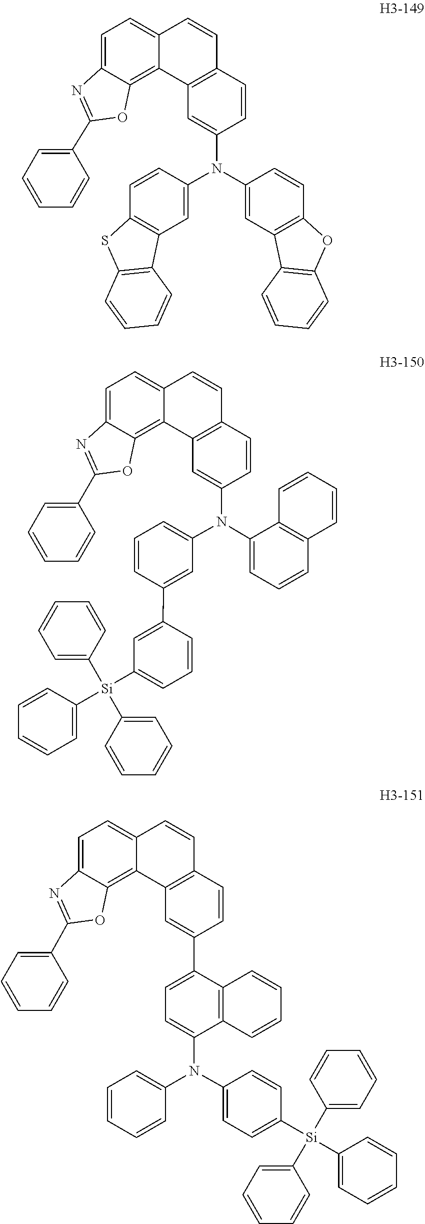 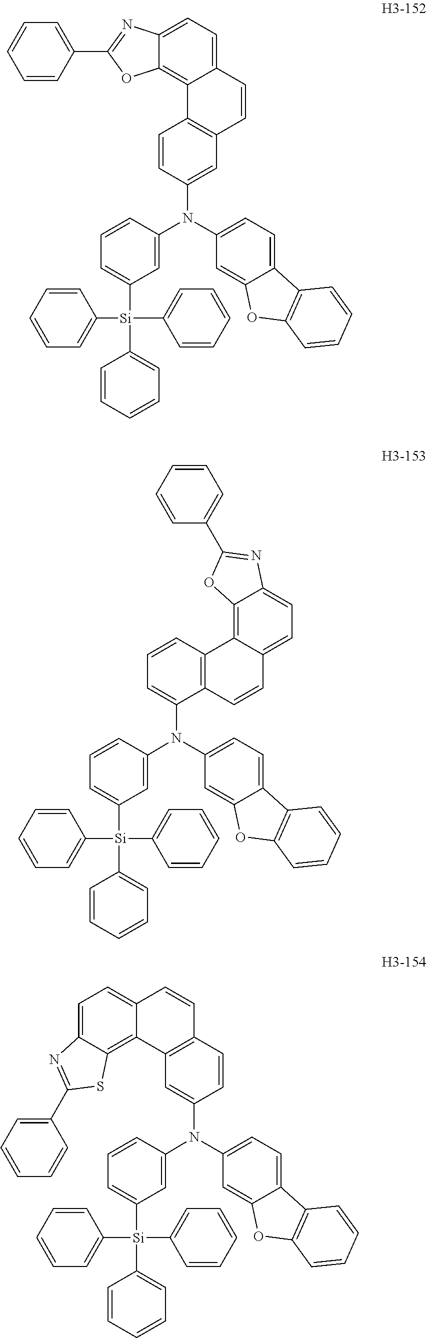 [SMILES: c1ccc(-c2nc3ccc4ccc5cc(N(c6cccc([Si](c7ccccc7)(c7ccccc7)c7ccccc7)c6)c6ccc7c(c6)oc6ccccc67)ccc5c4c3o2)cc1 ; c1ccc(-c2nc3ccc4ccc5c(N(c6cccc([Si](c7ccccc7)(c7ccccc7)c7ccccc7)c6)c6ccc7c(c6)oc6ccccc67)cccc5c4c3o2)cc1 ; c1ccc(-c2nc3ccc4ccc5ccc(N(c6cccc([Si](c7ccccc7)(c7ccccc7)c7ccccc7)c6)c6ccc7c(c6)oc6ccccc67)cc5c4c3s2)cc1] 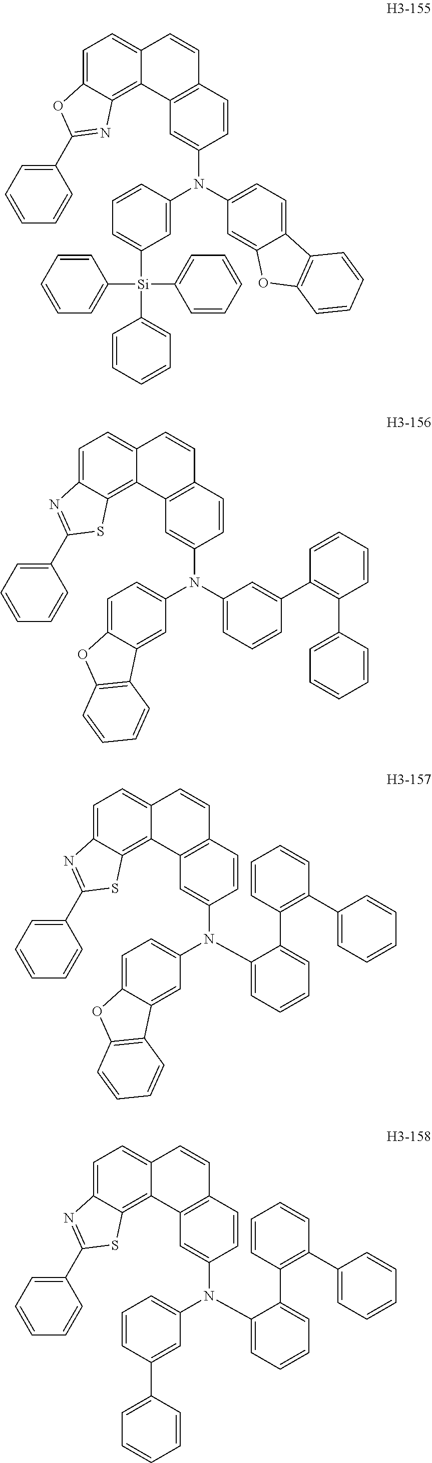 [SMILES: c1ccc(-c2nc3c(ccc4ccc5ccc(N(c6cccc([Si](c7ccccc7)(c7ccccc7)c7ccccc7)c6)c6ccc7c(c6)oc6ccccc67)cc5c43)o2)cc1 ; c1ccc(-c2nc3ccc4ccc5ccc(N(c6cccc(-c7ccccc7-c7ccccc7)c6)c6ccc7oc8ccccc8c7c6)cc5c4c3s2)cc1 ; c1ccc(-c2nc3ccc4ccc5ccc(N(c6ccc7oc8ccccc8c7c6)c6ccccc6-c6ccccc6-c6ccccc6)cc5c4c3s2)cc1 ; c1ccc(-c2cccc(N(c3ccc4ccc5ccc6nc(-c7ccccc7)sc6c5c4c3)c3ccccc3-c3ccccc3-c3ccccc3)c2)cc1] 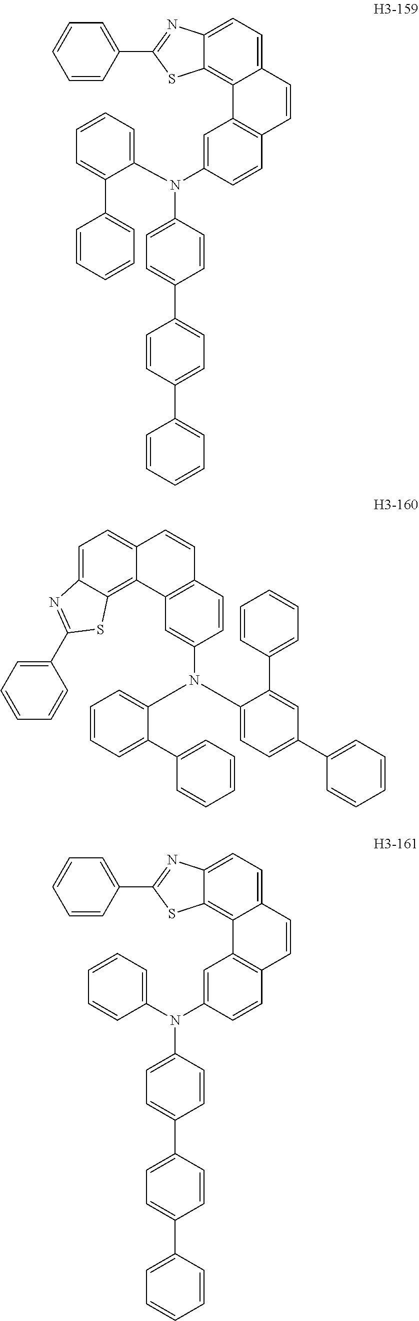 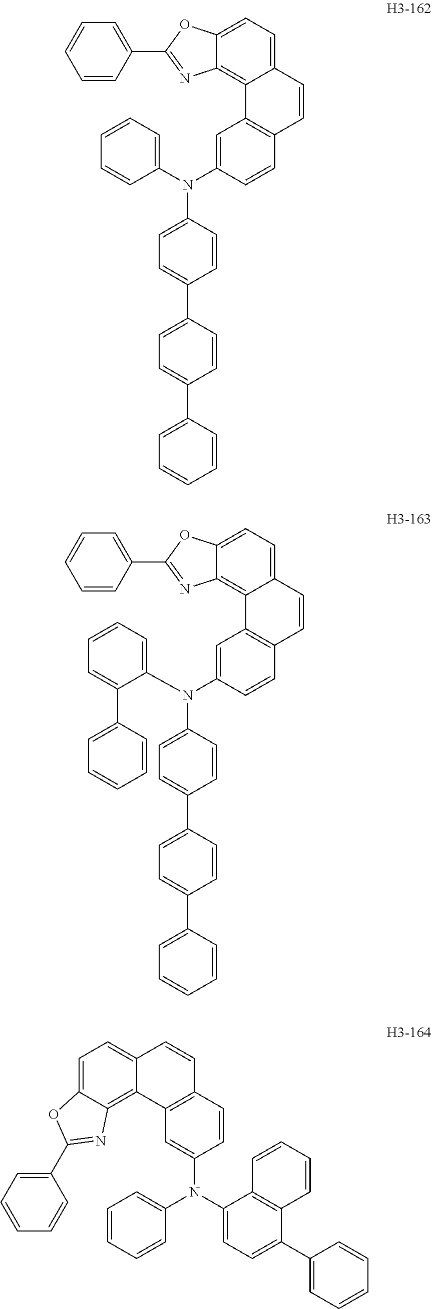 [SMILES: c1ccc(-c2ccc(-c3ccc(N(c4ccccc4)c4ccc5ccc6ccc7oc(-c8ccccc8)nc7c6c5c4)cc3)cc2)cc1 ; c1ccc(-c2ccc(-c3ccc(N(c4ccc5ccc6ccc7oc(-c8ccccc8)nc7c6c5c4)c4ccccc4-c4ccccc4)cc3)cc2)cc1 ; c1ccc(-c2nc3c(ccc4ccc5ccc(N(c6ccccc6)c6ccc(-c7ccccc7)c7ccccc67)cc5c43)o2)cc1] 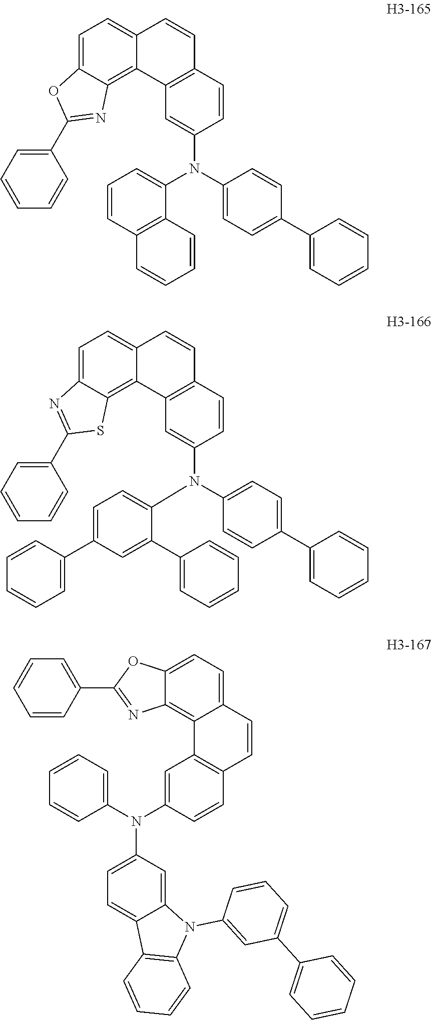 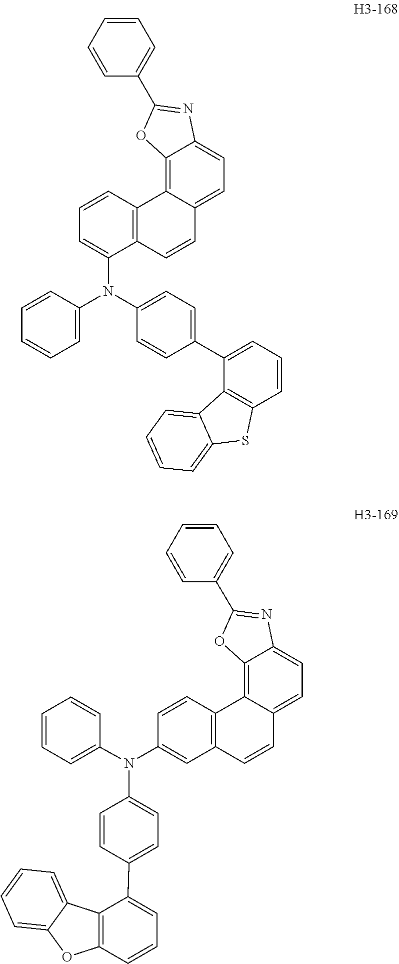 [SMILES: c1ccc(-c2nc3ccc4ccc5c(N(c6ccccc6)c6ccc(-c7cccc8sc9ccccc9c78)cc6)cccc5c4c3o2)cc1 ; c1ccc(-c2nc3ccc4ccc5cc(N(c6ccccc6)c6ccc(-c7cccc8oc9ccccc9c78)cc6)ccc5c4c3o2)cc1] 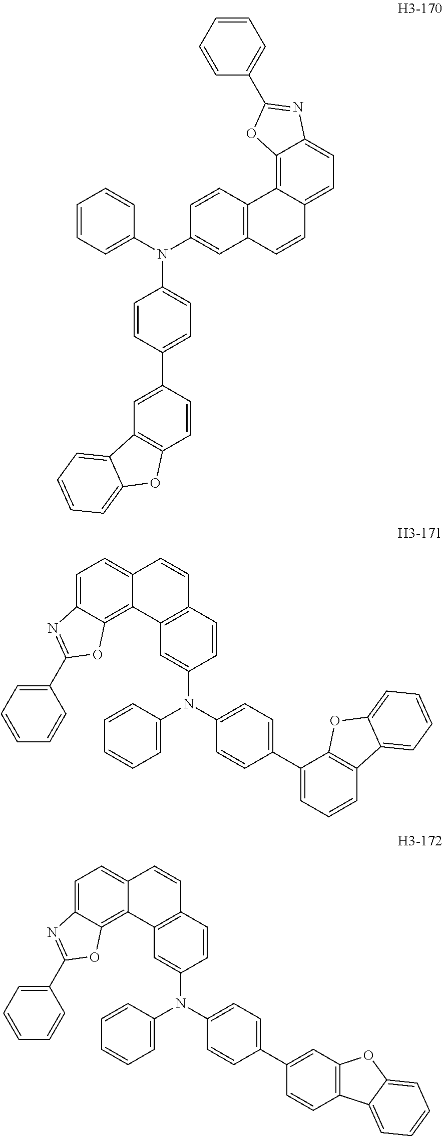 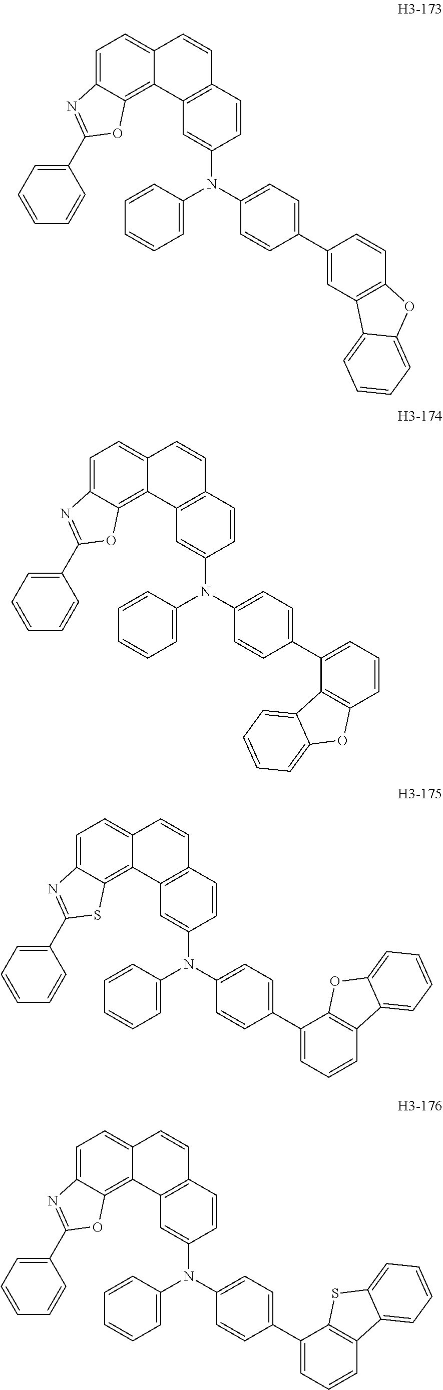 [SMILES: c1ccc(-c2nc3ccc4ccc5ccc(N(c6ccccc6)c6ccc(-c7ccc8oc9ccccc9c8c7)cc6)cc5c4c3o2)cc1 ; c1ccc(-c2nc3ccc4ccc5ccc(N(c6ccccc6)c6ccc(-c7cccc8oc9ccccc9c78)cc6)cc5c4c3o2)cc1 ; c1ccc(-c2nc3ccc4ccc5ccc(N(c6ccccc6)c6ccc(-c7cccc8c7oc7ccccc78)cc6)cc5c4c3s2)cc1 ; c1ccc(-c2nc3ccc4ccc5ccc(N(c6ccccc6)c6ccc(-c7cccc8c7sc7ccccc78)cc6)cc5c4c3o2)cc1] 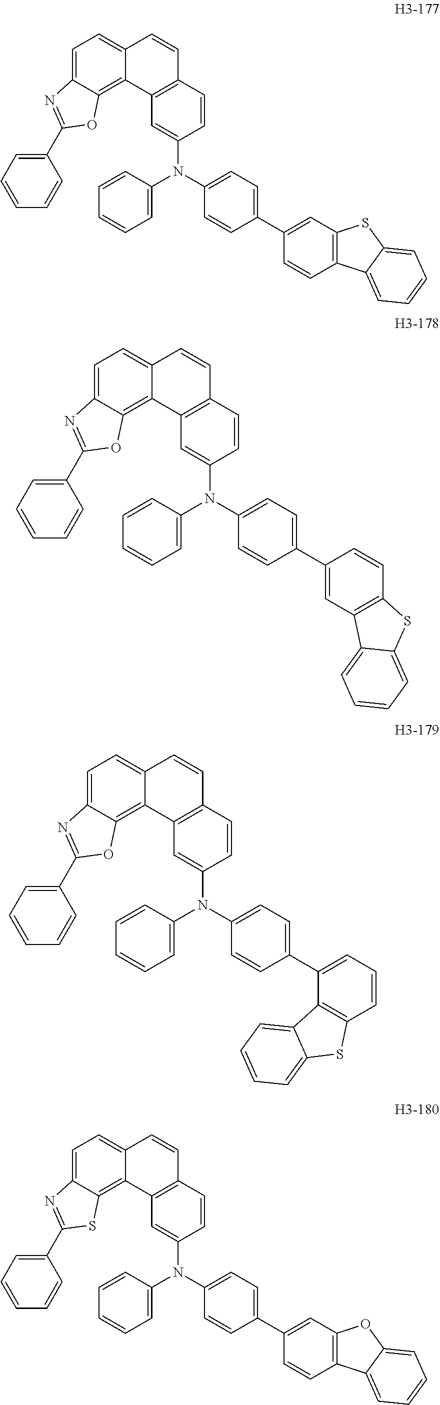 [SMILES: c1ccc(-c2nc3ccc4ccc5ccc(N(c6ccccc6)c6ccc(-c7ccc8c(c7)sc7ccccc78)cc6)cc5c4c3o2)cc1 ; c1ccc(-c2nc3ccc4ccc5ccc(N(c6ccccc6)c6ccc(-c7ccc8sc9ccccc9c8c7)cc6)cc5c4c3o2)cc1 ; c1ccc(-c2nc3ccc4ccc5ccc(N(c6ccccc6)c6ccc(-c7cccc8sc9ccccc9c78)cc6)cc5c4c3o2)cc1 ; c1ccc(-c2nc3ccc4ccc5ccc(N(c6ccccc6)c6ccc(-c7ccc8c(c7)oc7ccccc78)cc6)cc5c4c3s2)cc1] 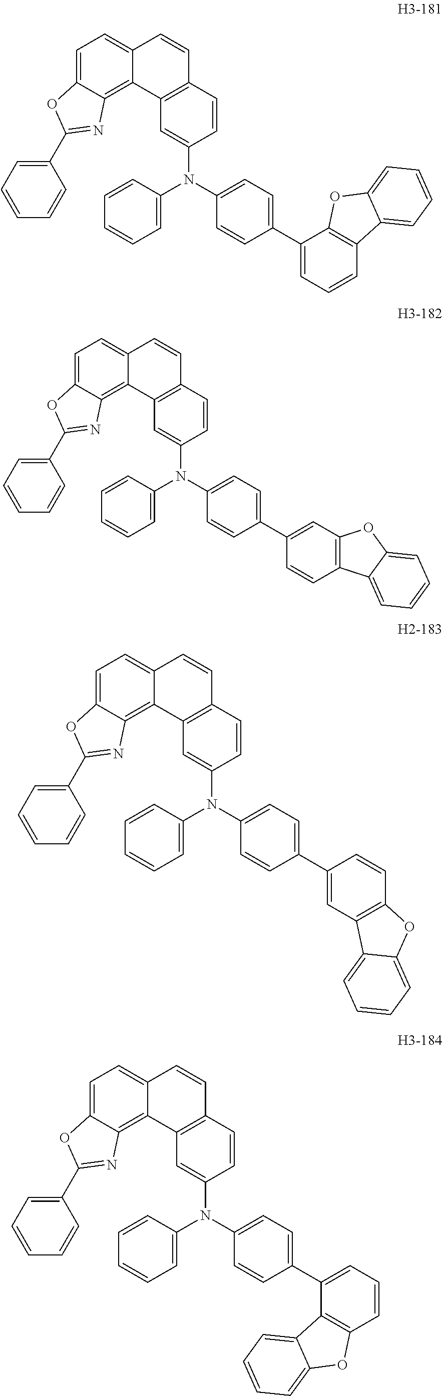 [SMILES: c1ccc(-c2nc3c(ccc4ccc5ccc(N(c6ccccc6)c6ccc(-c7cccc8c7oc7ccccc78)cc6)cc5c43)o2)cc1 ; c1ccc(-c2nc3c(ccc4ccc5ccc(N(c6ccccc6)c6ccc(-c7ccc8c(c7)oc7ccccc78)cc6)cc5c43)o2)cc1 ; c1ccc(-c2nc3c(ccc4ccc5ccc(N(c6ccccc6)c6ccc(-c7ccc8oc9ccccc9c8c7)cc6)cc5c43)o2)cc1 ; c1ccc(-c2nc3c(ccc4ccc5ccc(N(c6ccccc6)c6ccc(-c7cccc8oc9ccccc9c78)cc6)cc5c43)o2)cc1] 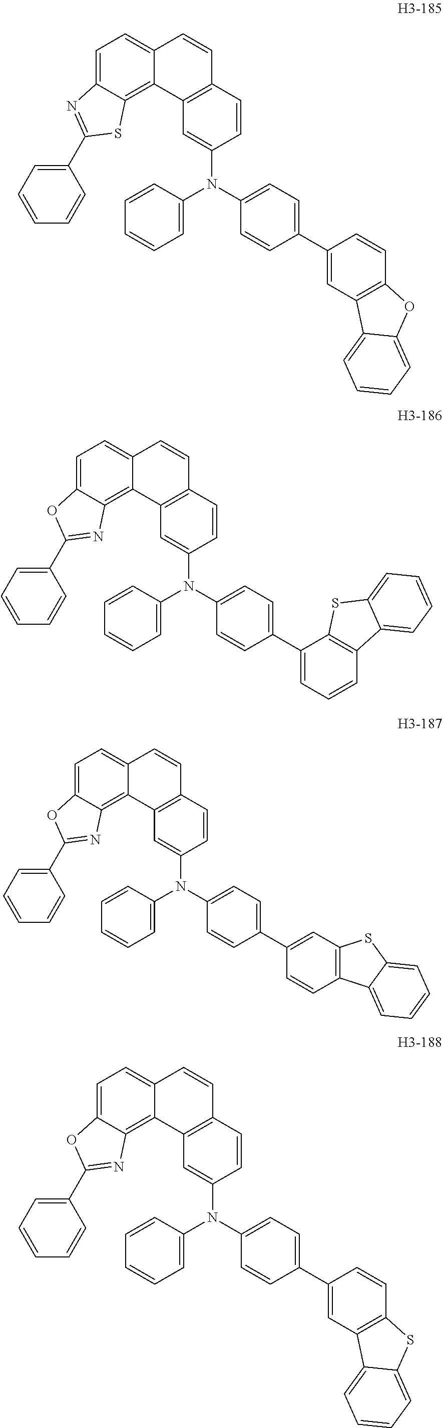 [SMILES: c1ccc(-c2nc3ccc4ccc5ccc(N(c6ccccc6)c6ccc(-c7ccc8oc9ccccc9c8c7)cc6)cc5c4c3s2)cc1 ; c1ccc(-c2nc3c(ccc4ccc5ccc(N(c6ccccc6)c6ccc(-c7cccc8c7sc7ccccc78)cc6)cc5c43)o2)cc1 ; c1ccc(-c2nc3c(ccc4ccc5ccc(N(c6ccccc6)c6ccc(-c7ccc8c(c7)sc7ccccc78)cc6)cc5c43)o2)cc1 ; c1ccc(-c2nc3c(ccc4ccc5ccc(N(c6ccccc6)c6ccc(-c7ccc8sc9ccccc9c8c7)cc6)cc5c43)o2)cc1] 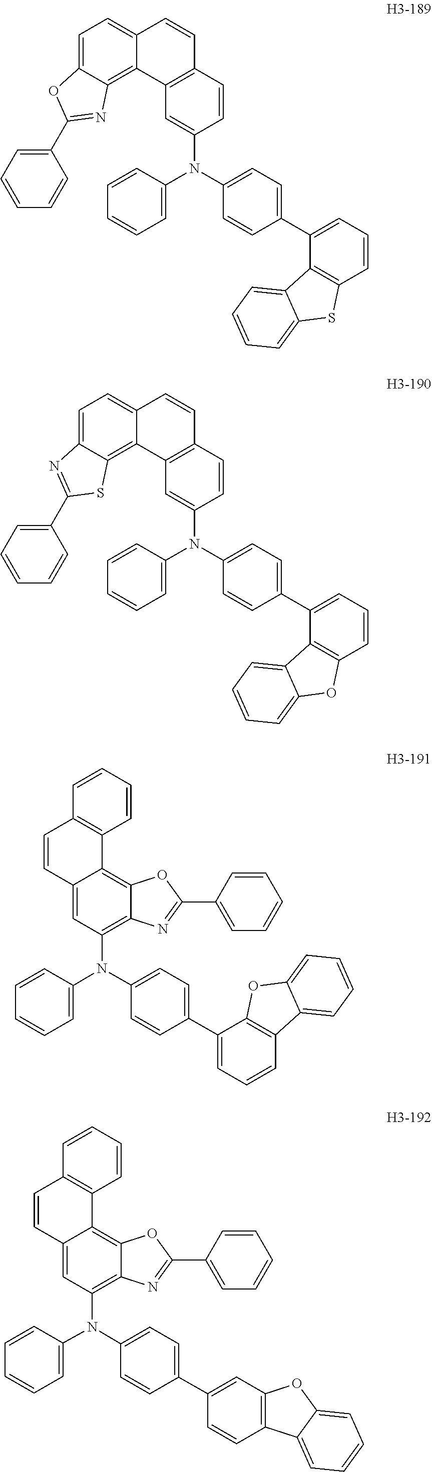 [SMILES: c1ccc(-c2nc3c(ccc4ccc5ccc(N(c6ccccc6)c6ccc(-c7cccc8sc9ccccc9c78)cc6)cc5c43)o2)cc1 ; c1ccc(-c2nc3ccc4ccc5ccc(N(c6ccccc6)c6ccc(-c7cccc8oc9ccccc9c78)cc6)cc5c4c3s2)cc1 ; c1ccc(-c2nc3c(N(c4ccccc4)c4ccc(-c5cccc6c5oc5ccccc56)cc4)cc4ccc5ccccc5c4c3o2)cc1 ; c1ccc(-c2nc3c(N(c4ccccc4)c4ccc(-c5ccc6c(c5)oc5ccccc56)cc4)cc4ccc5ccccc5c4c3o2)cc1] 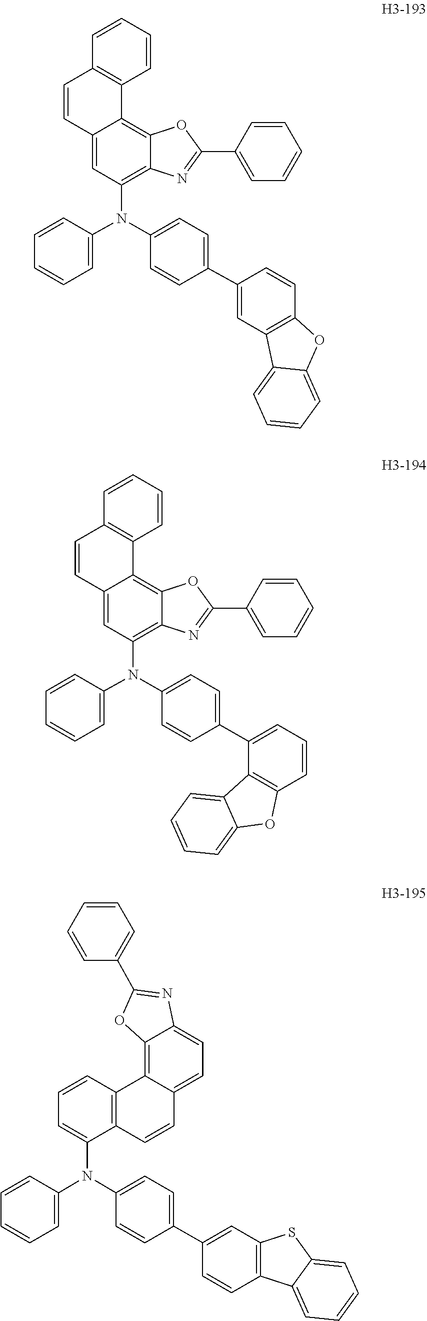 [SMILES: c1ccc(-c2nc3c(N(c4ccccc4)c4ccc(-c5ccc6oc7ccccc7c6c5)cc4)cc4ccc5ccccc5c4c3o2)cc1 ; c1ccc(-c2nc3c(N(c4ccccc4)c4ccc(-c5cccc6oc7ccccc7c56)cc4)cc4ccc5ccccc5c4c3o2)cc1 ; c1ccc(-c2nc3ccc4ccc5c(N(c6ccccc6)c6ccc(-c7ccc8c(c7)sc7ccccc78)cc6)cccc5c4c3o2)cc1] 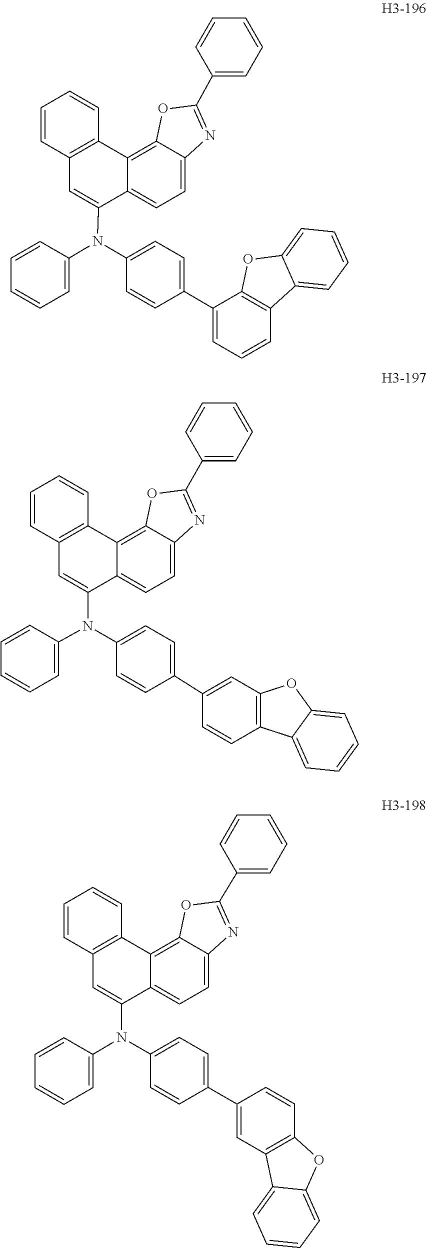 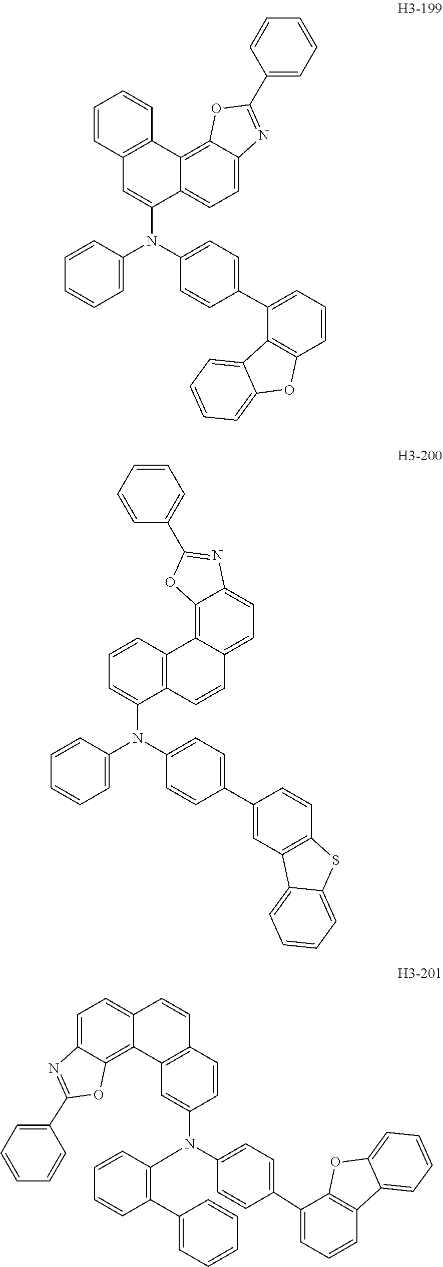 [SMILES: c1ccc(-c2nc3ccc4c(N(c5ccccc5)c5ccc(-c6cccc7oc8ccccc8c67)cc5)cc5ccccc5c4c3o2)cc1 ; c1ccc(-c2nc3ccc4ccc5c(N(c6ccccc6)c6ccc(-c7ccc8sc9ccccc9c8c7)cc6)cccc5c4c3o2)cc1 ; c1ccc(-c2nc3ccc4ccc5ccc(N(c6ccc(-c7cccc8c7oc7ccccc78)cc6)c6ccccc6-c6ccccc6)cc5c4c3o2)cc1] 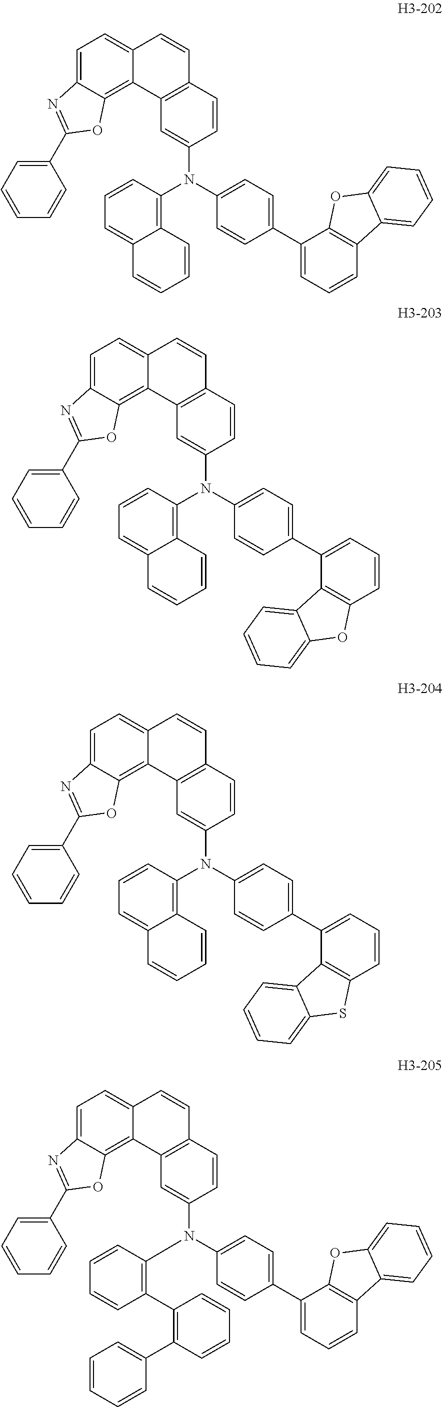 [SMILES: c1ccc(-c2nc3ccc4ccc5ccc(N(c6ccc(-c7cccc8c7oc7ccccc78)cc6)c6cccc7ccccc67)cc5c4c3o2)cc1 ; c1ccc(-c2nc3ccc4ccc5ccc(N(c6ccc(-c7cccc8oc9ccccc9c78)cc6)c6cccc7ccccc67)cc5c4c3o2)cc1 ; c1ccc(-c2nc3ccc4ccc5ccc(N(c6ccc(-c7cccc8sc9ccccc9c78)cc6)c6cccc7ccccc67)cc5c4c3o2)cc1 ; c1ccc(-c2nc3ccc4ccc5ccc(N(c6ccc(-c7cccc8c7oc7ccccc78)cc6)c6ccccc6-c6ccccc6-c6ccccc6)cc5c4c3o2)cc1] 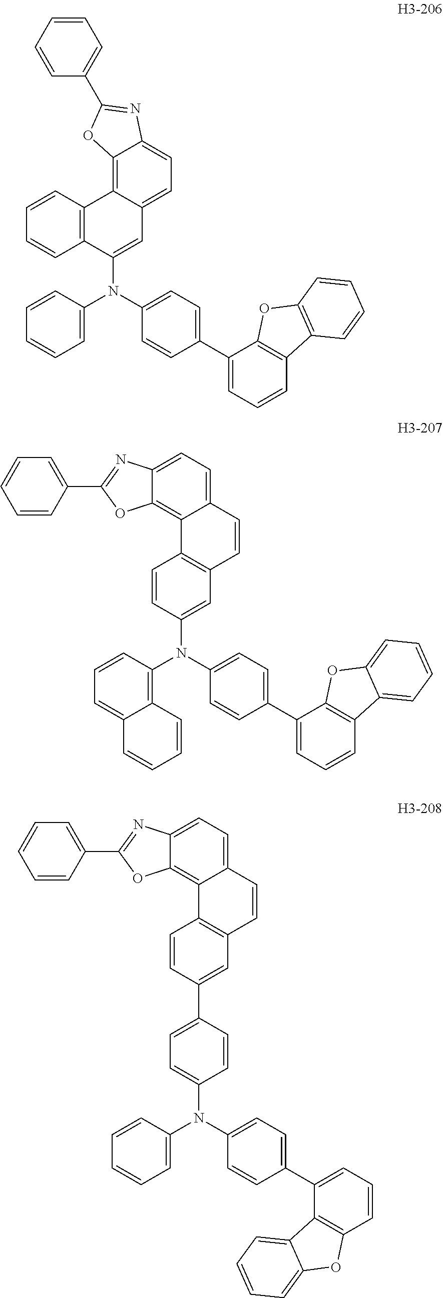 [SMILES: c1ccc(-c2nc3ccc4cc(N(c5ccccc5)c5ccc(-c6cccc7c6oc6ccccc67)cc5)c5ccccc5c4c3o2)cc1 ; c1ccc(-c2nc3ccc4ccc5cc(N(c6ccc(-c7cccc8c7oc7ccccc78)cc6)c6cccc7ccccc67)ccc5c4c3o2)cc1 ; c1ccc(-c2nc3ccc4ccc5cc(-c6ccc(N(c7ccccc7)c7ccc(-c8cccc9oc%10ccccc%10c89)cc7)cc6)ccc5c4c3o2)cc1] 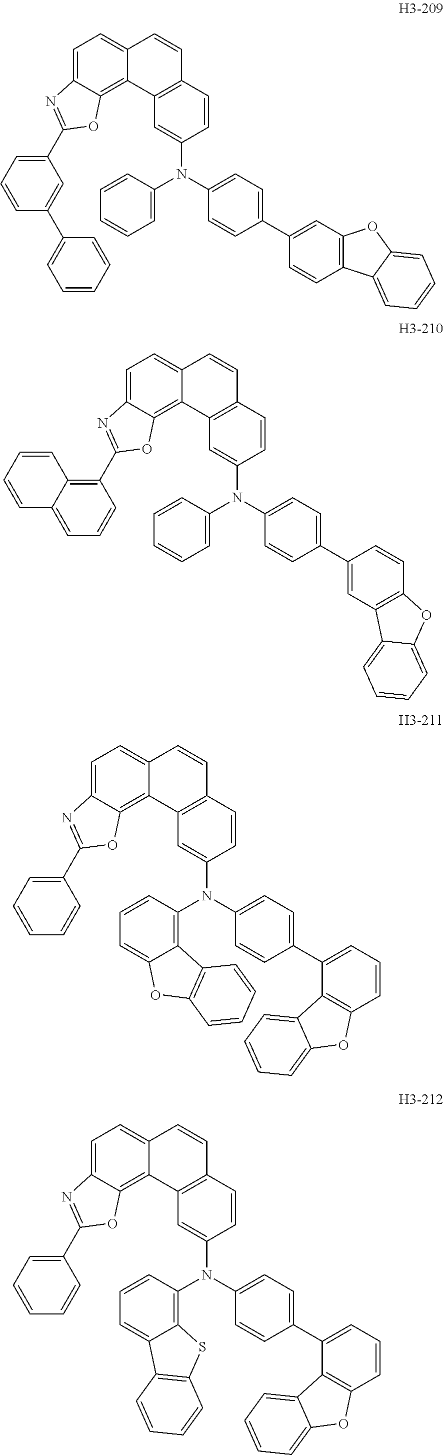 [SMILES: c1ccc(-c2cccc(-c3nc4ccc5ccc6ccc(N(c7ccccc7)c7ccc(-c8ccc9c(c8)oc8ccccc89)cc7)cc6c5c4o3)c2)cc1 ; c1ccc(N(c2ccc(-c3ccc4oc5ccccc5c4c3)cc2)c2ccc3ccc4ccc5nc(-c6cccc7ccccc67)oc5c4c3c2)cc1 ; c1ccc(-c2nc3ccc4ccc5ccc(N(c6ccc(-c7cccc8oc9ccccc9c78)cc6)c6cccc7oc8ccccc8c67)cc5c4c3o2)cc1 ; c1ccc(-c2nc3ccc4ccc5ccc(N(c6ccc(-c7cccc8oc9ccccc9c78)cc6)c6cccc7c6sc6ccccc67)cc5c4c3o2)cc1] 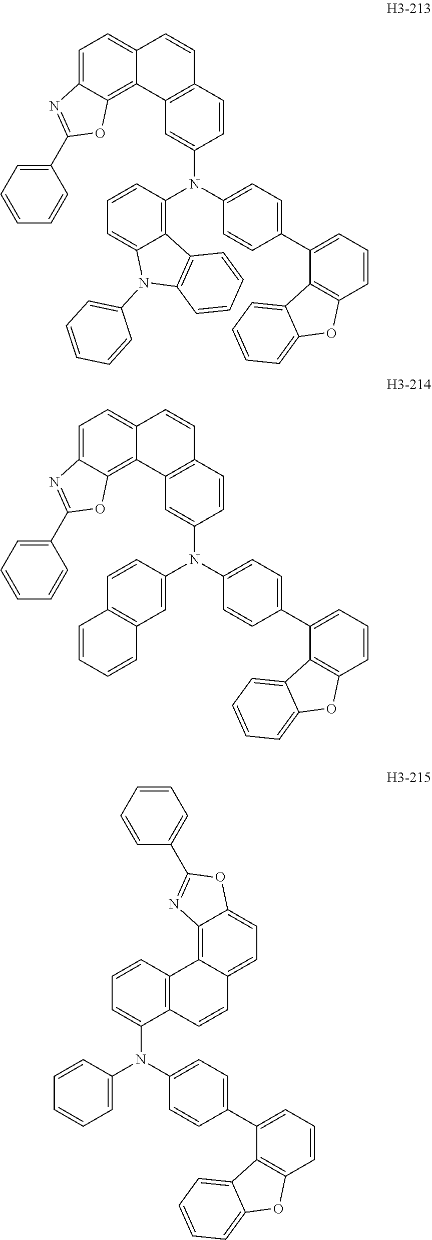 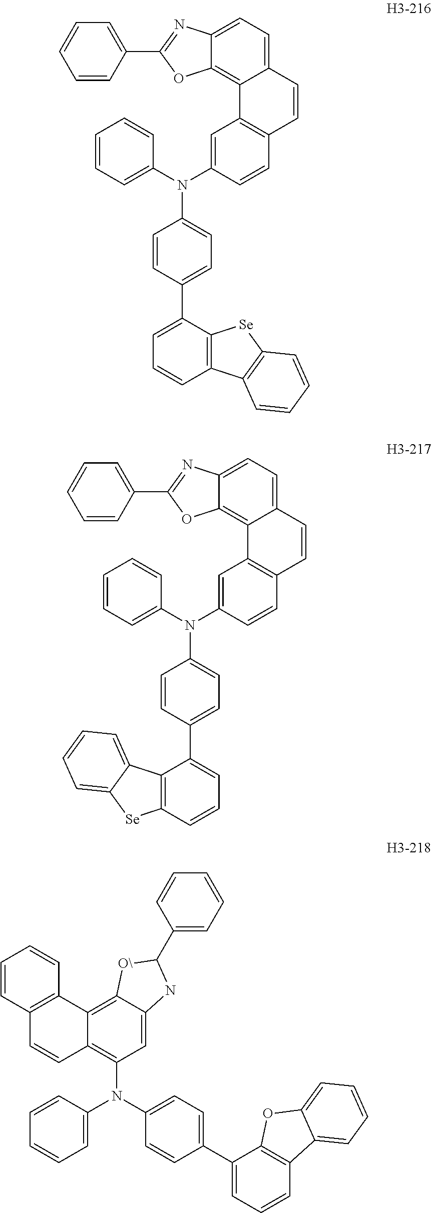 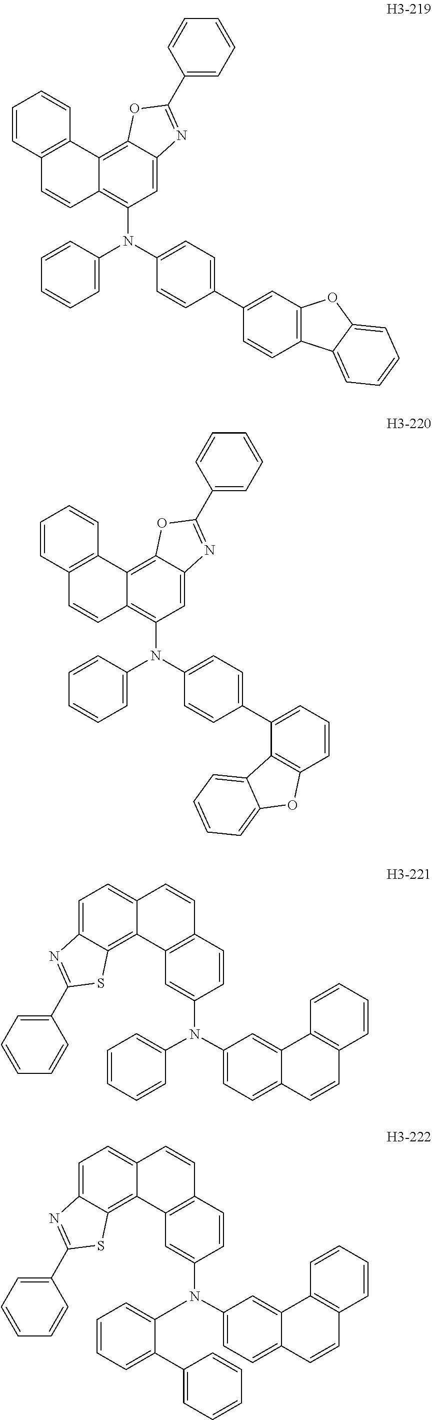 [SMILES: c1ccc(-c2nc3cc(N(c4ccccc4)c4ccc(-c5ccc6c(c5)oc5ccccc56)cc4)c4ccc5ccccc5c4c3o2)cc1 ; c1ccc(-c2nc3cc(N(c4ccccc4)c4ccc(-c5cccc6oc7ccccc7c56)cc4)c4ccc5ccccc5c4c3o2)cc1 ; c1ccc(-c2nc3ccc4ccc5ccc(N(c6ccccc6)c6ccc7ccc8ccccc8c7c6)cc5c4c3s2)cc1 ; c1ccc(-c2nc3ccc4ccc5ccc(N(c6ccc7ccc8ccccc8c7c6)c6ccccc6-c6ccccc6)cc5c4c3s2)cc1] 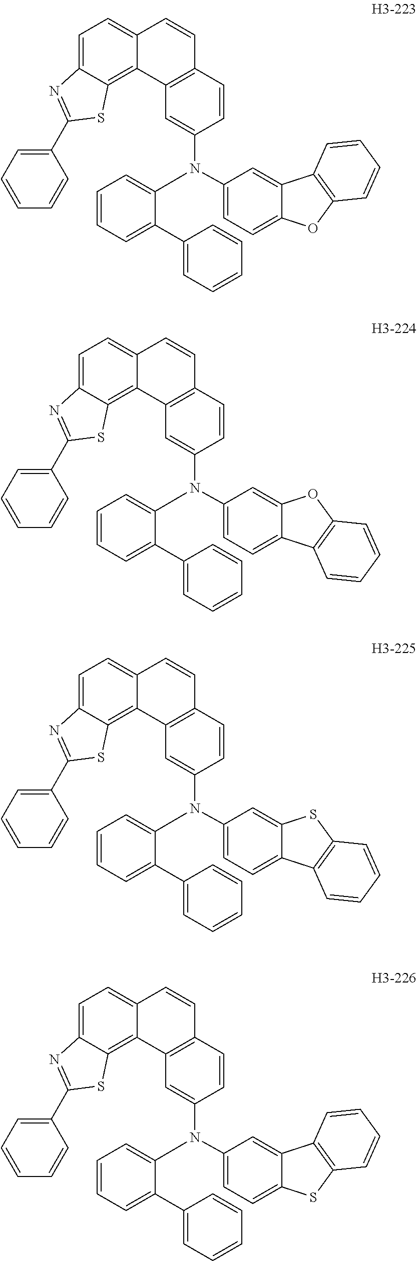 [SMILES: c1ccc(-c2nc3ccc4ccc5ccc(N(c6ccc7oc8ccccc8c7c6)c6ccccc6-c6ccccc6)cc5c4c3s2)cc1 ; c1ccc(-c2nc3ccc4ccc5ccc(N(c6ccc7c(c6)oc6ccccc67)c6ccccc6-c6ccccc6)cc5c4c3s2)cc1 ; c1ccc(-c2nc3ccc4ccc5ccc(N(c6ccc7c(c6)sc6ccccc67)c6ccccc6-c6ccccc6)cc5c4c3s2)cc1 ; c1ccc(-c2nc3ccc4ccc5ccc(N(c6ccc7sc8ccccc8c7c6)c6ccccc6-c6ccccc6)cc5c4c3s2)cc1] 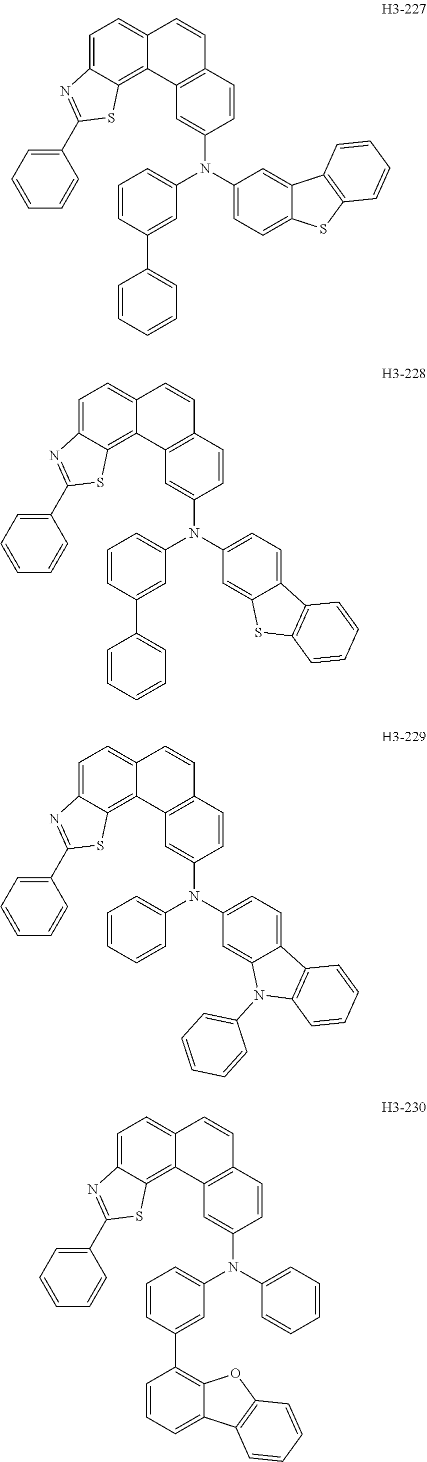 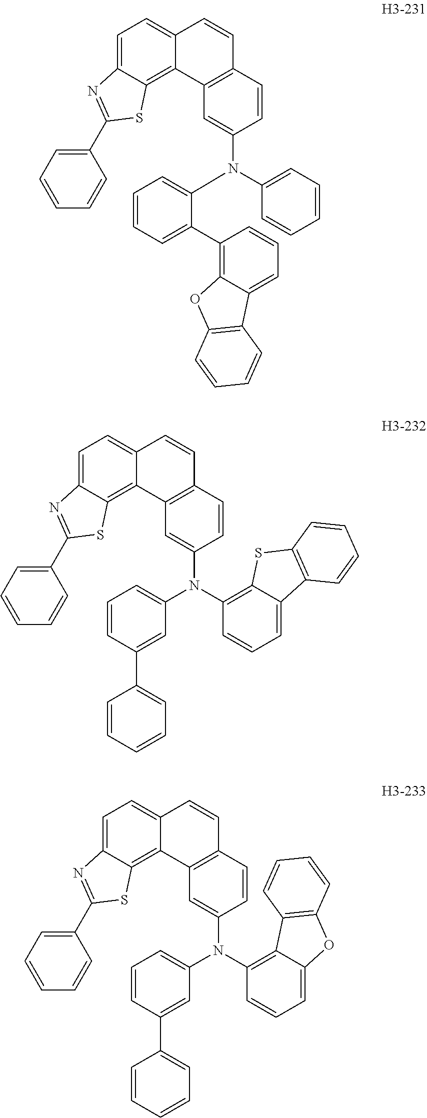 [SMILES: c1ccc(-c2nc3ccc4ccc5ccc(N(c6ccccc6)c6ccccc6-c6cccc7c6oc6ccccc67)cc5c4c3s2)cc1 ; c1ccc(-c2cccc(N(c3ccc4ccc5ccc6nc(-c7ccccc7)sc6c5c4c3)c3cccc4c3sc3ccccc34)c2)cc1 ; c1ccc(-c2cccc(N(c3ccc4ccc5ccc6nc(-c7ccccc7)sc6c5c4c3)c3cccc4oc5ccccc5c34)c2)cc1] 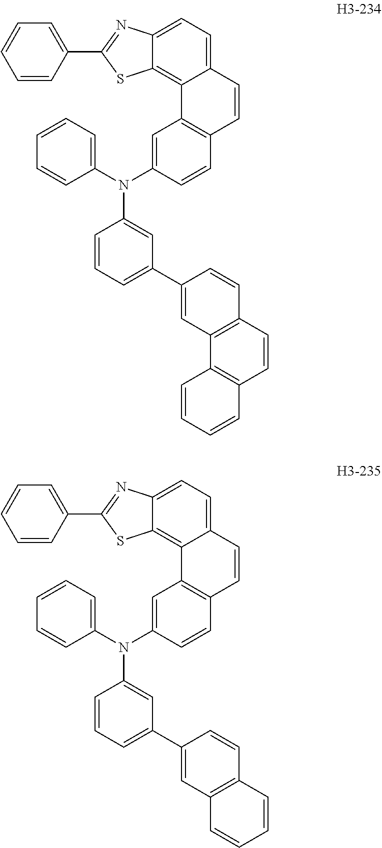 [SMILES: c1ccc(-c2nc3ccc4ccc5ccc(N(c6ccccc6)c6cccc(-c7ccc8ccc9ccccc9c8c7)c6)cc5c4c3s2)cc1 ; c1ccc(-c2nc3ccc4ccc5ccc(N(c6ccccc6)c6cccc(-c7ccc8ccccc8c7)c6)cc5c4c3s2)cc1] 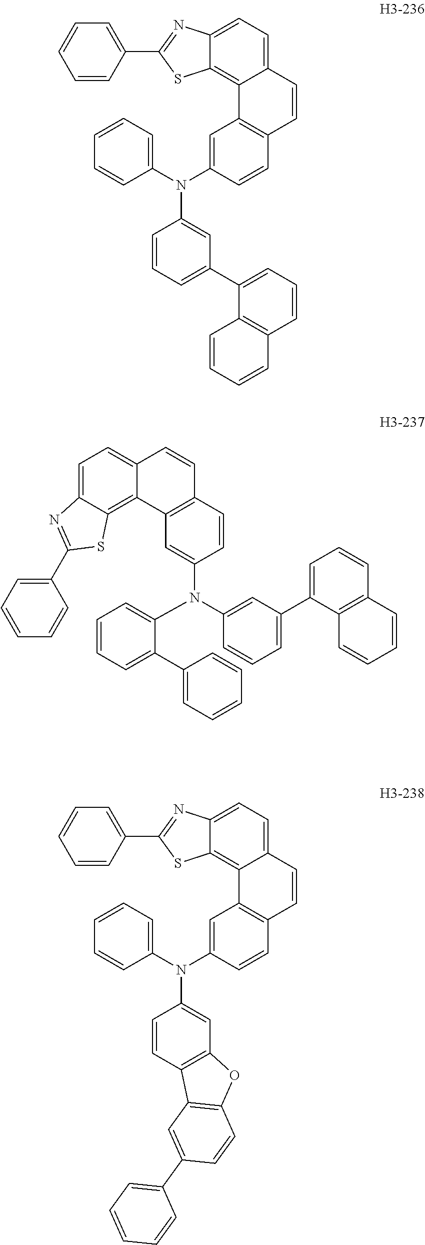 [SMILES: c1ccc(-c2nc3ccc4ccc5ccc(N(c6ccccc6)c6cccc(-c7cccc8ccccc78)c6)cc5c4c3s2)cc1 ; c1ccc(-c2nc3ccc4ccc5ccc(N(c6cccc(-c7cccc8ccccc78)c6)c6ccccc6-c6ccccc6)cc5c4c3s2)cc1 ; c1ccc(-c2ccc3oc4cc(N(c5ccccc5)c5ccc6ccc7ccc8nc(-c9ccccc9)sc8c7c6c5)ccc4c3c2)cc1] 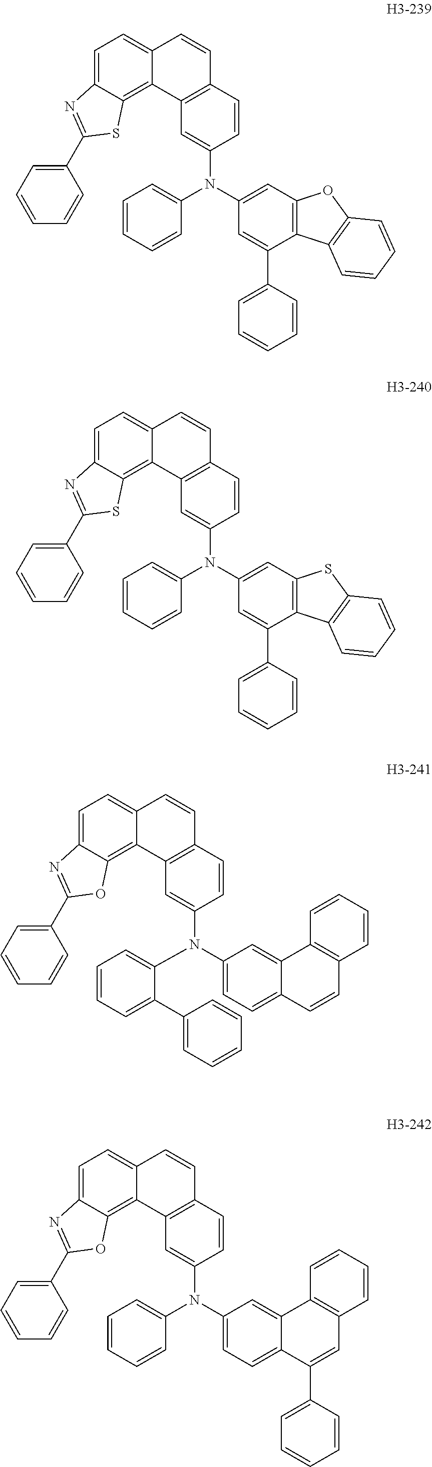 [SMILES: c1ccc(-c2nc3ccc4ccc5ccc(N(c6ccccc6)c6cc(-c7ccccc7)c7c(c6)oc6ccccc67)cc5c4c3s2)cc1 ; c1ccc(-c2nc3ccc4ccc5ccc(N(c6ccccc6)c6cc(-c7ccccc7)c7c(c6)sc6ccccc67)cc5c4c3s2)cc1 ; c1ccc(-c2nc3ccc4ccc5ccc(N(c6ccc7ccc8ccccc8c7c6)c6ccccc6-c6ccccc6)cc5c4c3o2)cc1 ; c1ccc(-c2nc3ccc4ccc5ccc(N(c6ccccc6)c6ccc7c(-c8ccccc8)cc8ccccc8c7c6)cc5c4c3o2)cc1] 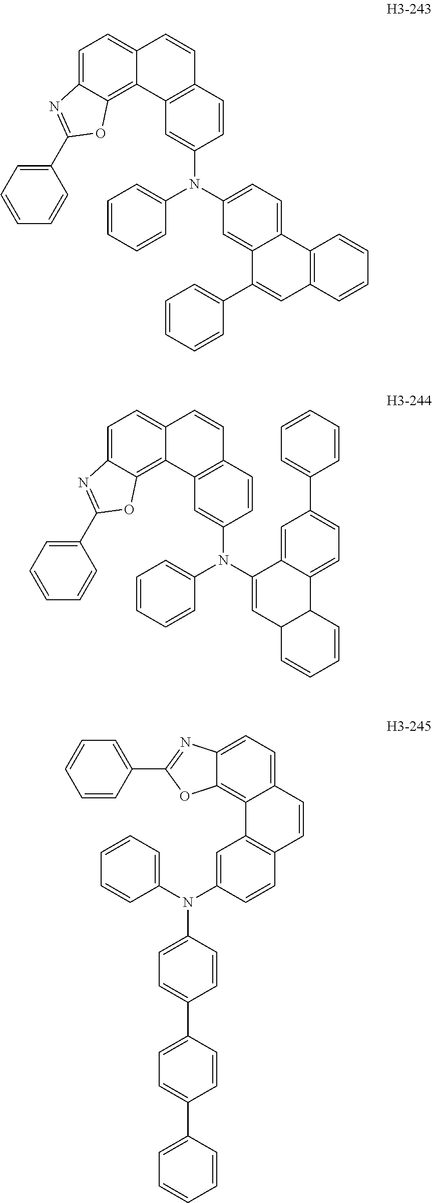 [SMILES: c1ccc(-c2nc3ccc4ccc5ccc(N(c6ccccc6)c6ccc7c(c6)c(-c6ccccc6)cc6ccccc67)cc5c4c3o2)cc1 ; C1=CC2C=C(N(c3ccccc3)c3ccc4ccc5ccc6nc(-c7ccccc7)oc6c5c4c3)c3cc(-c4ccccc4)ccc3C2C=C1 ; c1ccc(-c2ccc(-c3ccc(N(c4ccccc4)c4ccc5ccc6ccc7nc(-c8ccccc8)oc7c6c5c4)cc3)cc2)cc1] 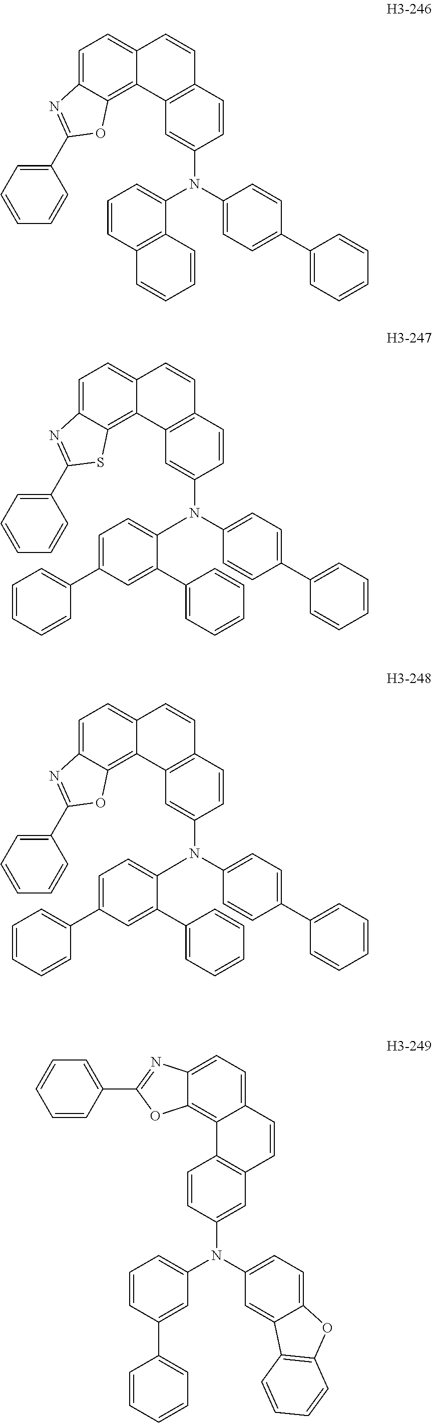 [SMILES: c1ccc(-c2ccc(N(c3ccc4ccc5ccc6nc(-c7ccccc7)oc6c5c4c3)c3cccc4ccccc34)cc2)cc1 ; c1ccc(-c2ccc(N(c3ccc4ccc5ccc6nc(-c7ccccc7)sc6c5c4c3)c3ccc(-c4ccccc4)cc3-c3ccccc3)cc2)cc1 ; c1ccc(-c2ccc(N(c3ccc4ccc5ccc6nc(-c7ccccc7)oc6c5c4c3)c3ccc(-c4ccccc4)cc3-c3ccccc3)cc2)cc1 ; c1ccc(-c2cccc(N(c3ccc4c(ccc5ccc6nc(-c7ccccc7)oc6c54)c3)c3ccc4oc5ccccc5c4c3)c2)cc1] 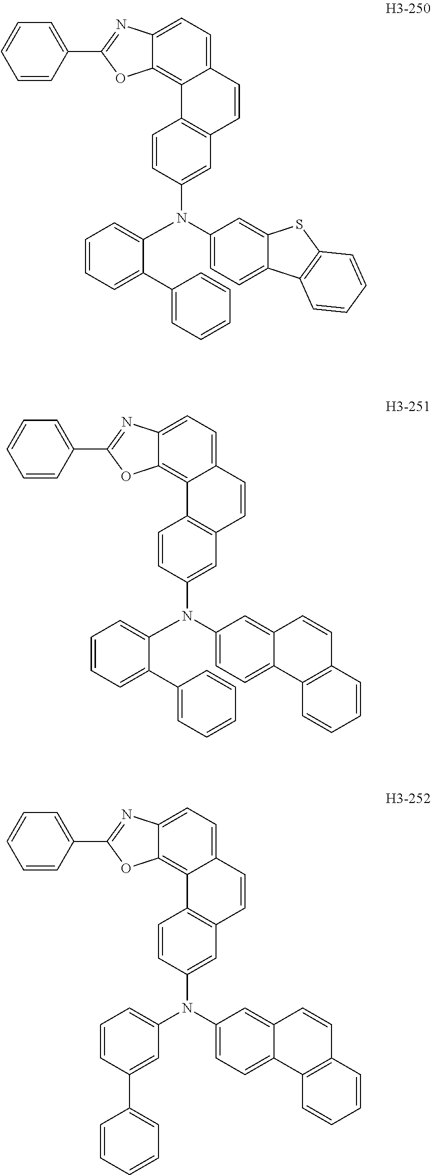 [SMILES: c1ccc(-c2nc3ccc4ccc5cc(N(c6ccc7c(c6)sc6ccccc67)c6ccccc6-c6ccccc6)ccc5c4c3o2)cc1 ; c1ccc(-c2nc3ccc4ccc5cc(N(c6ccc7c(ccc8ccccc87)c6)c6ccccc6-c6ccccc6)ccc5c4c3o2)cc1 ; c1ccc(-c2cccc(N(c3ccc4c(ccc5ccccc54)c3)c3ccc4c(ccc5ccc6nc(-c7ccccc7)oc6c54)c3)c2)cc1] 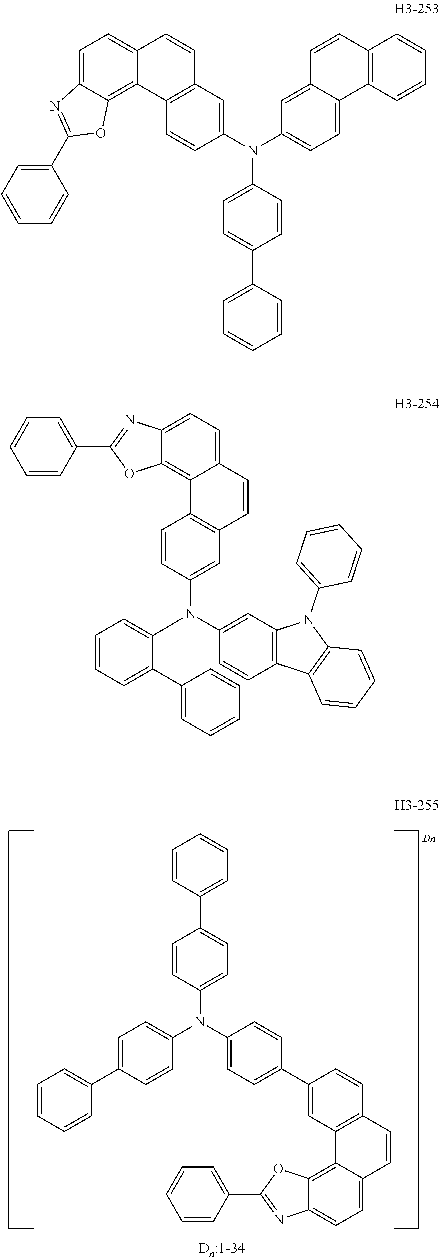 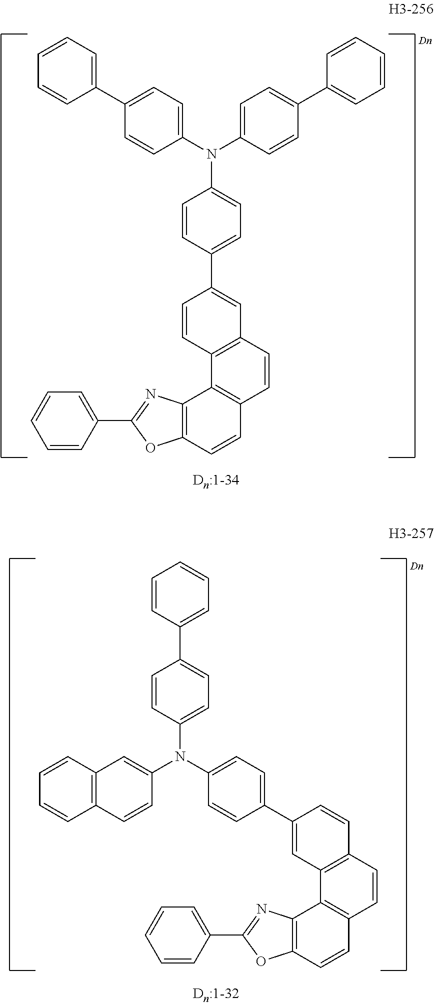 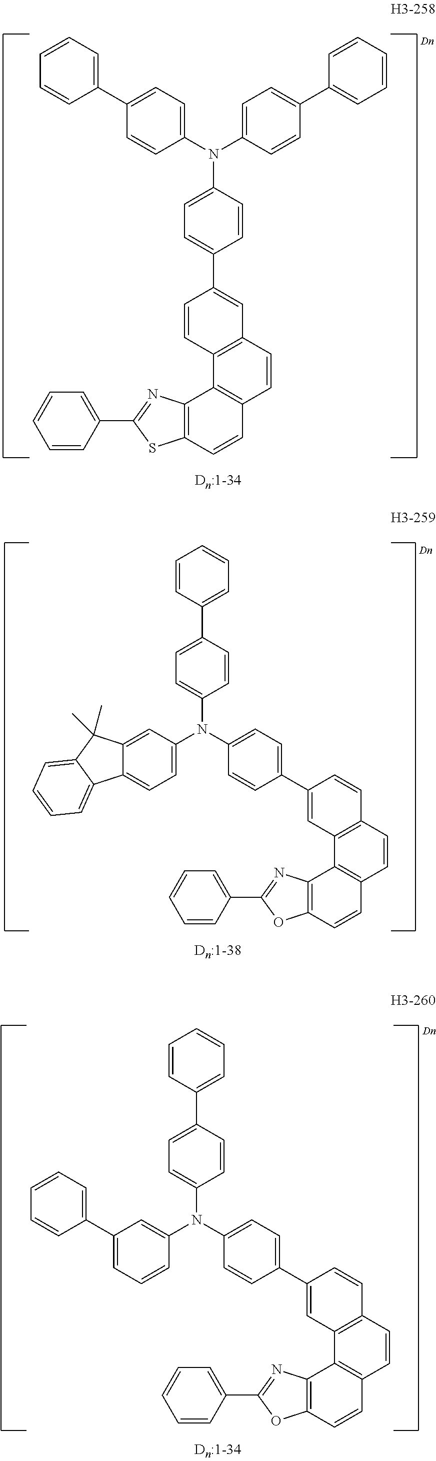 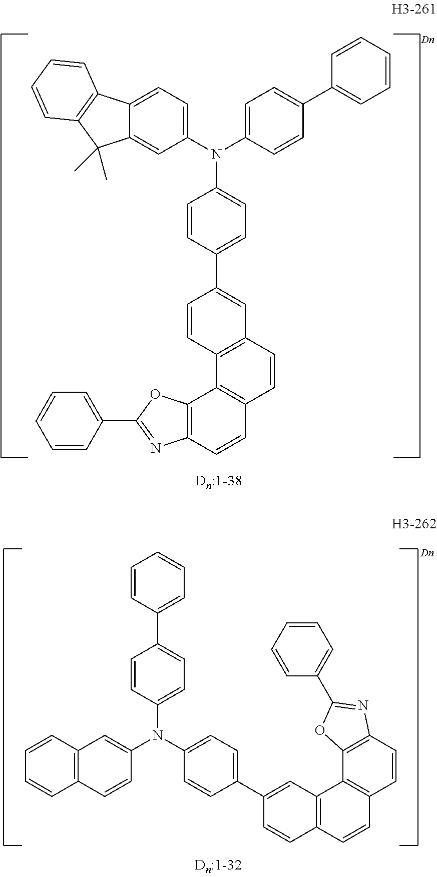 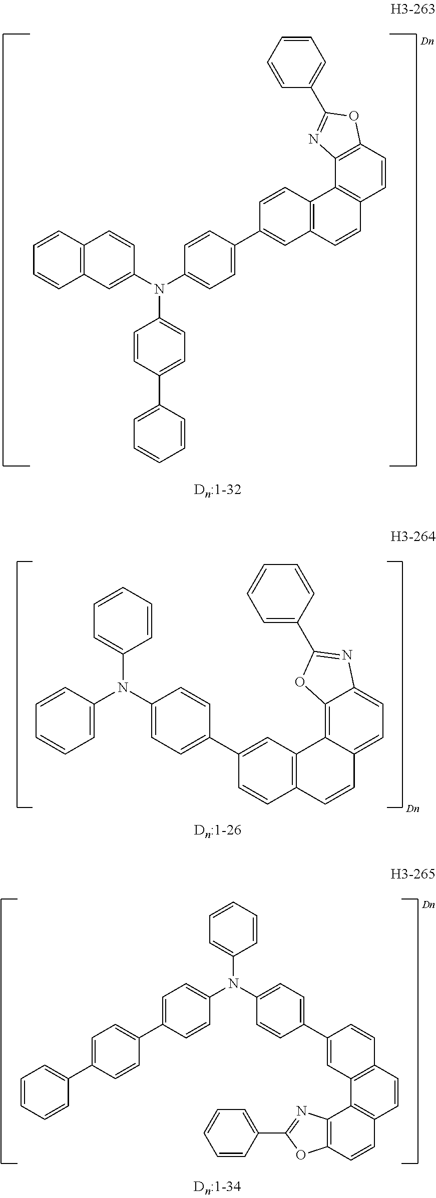 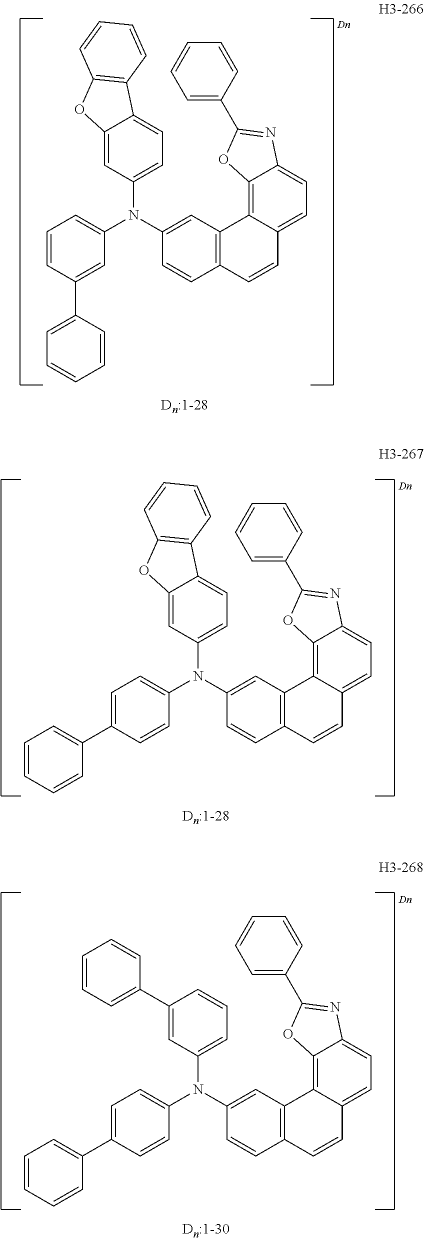 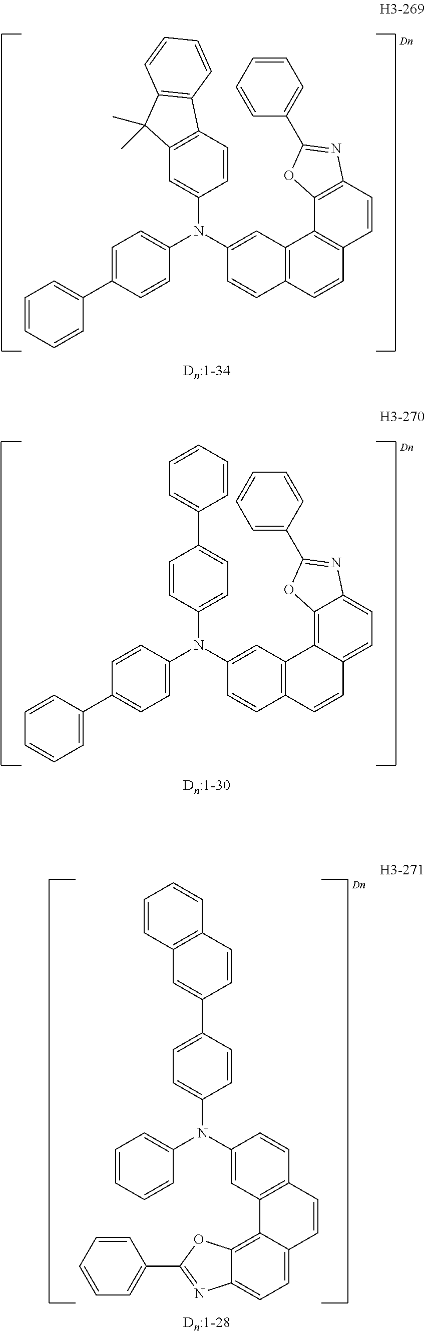 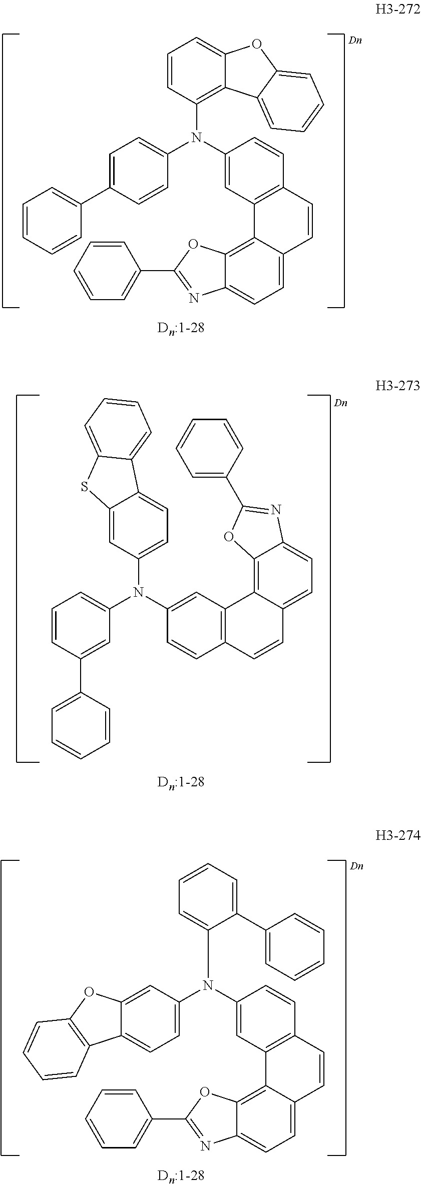 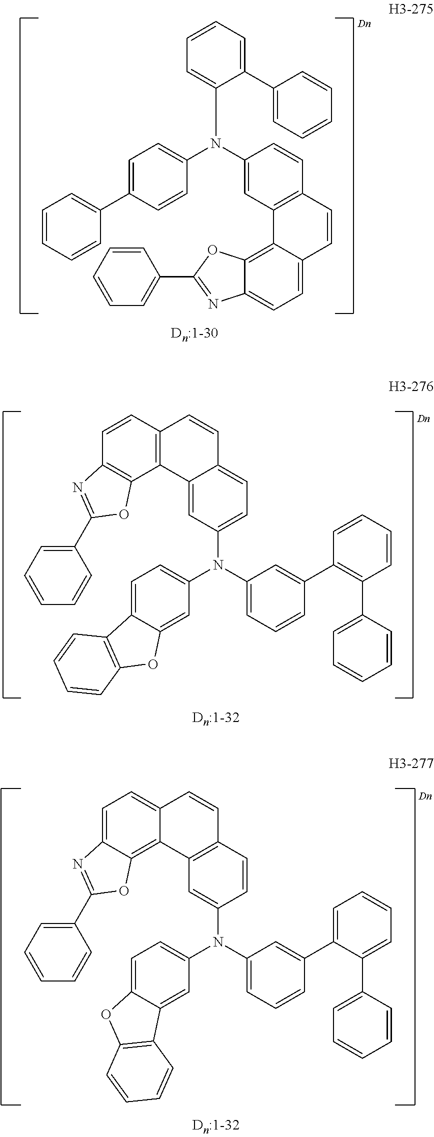 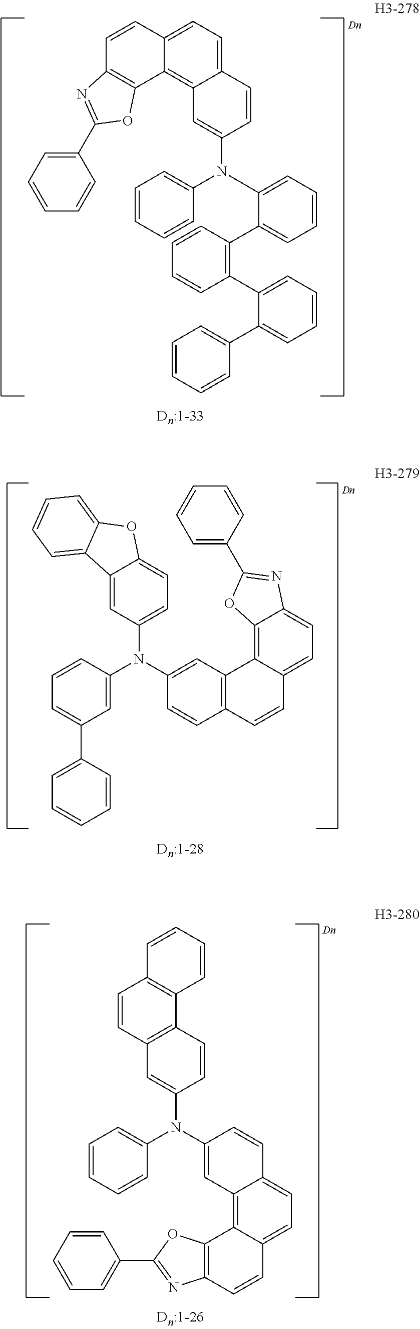 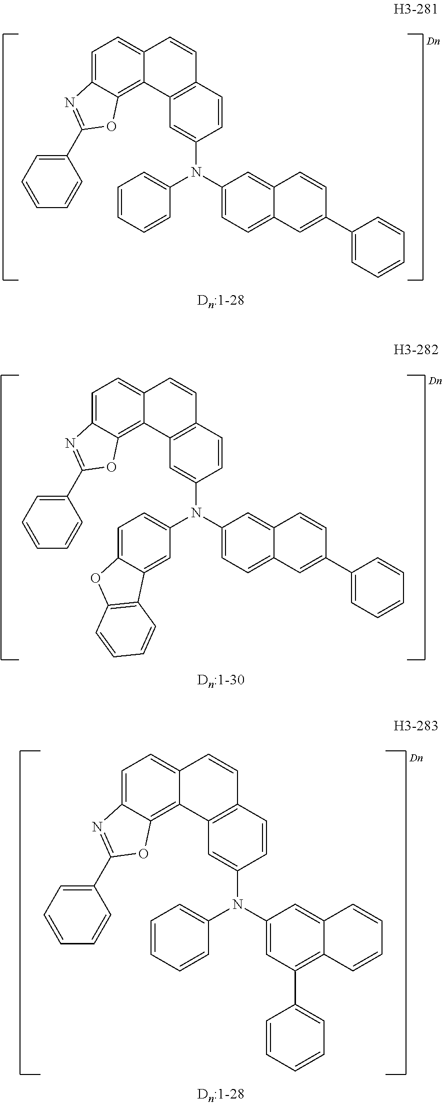 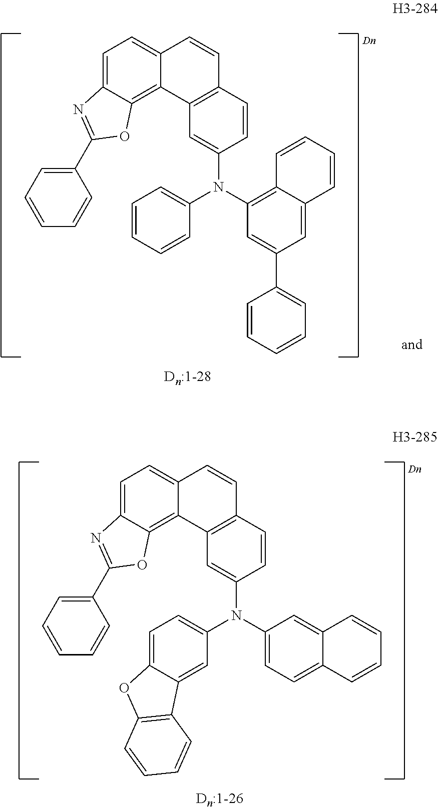
wherein in each of Compounds H3-255 to H3-285, Dₙ means that 1 to 26, 1 to 28, 1 to 30, 1 to 32, 1 to 33, 1 to 34, or 1 to 38 hydrogens are replaced with deuterium.

14. An organic electroluminescent device comprising a first electrode; a second electrode; and at least one light-emitting layer between the first electrode and the second electrode, wherein the at least one light-emitting layer comprises the plurality of host materials according to claim 9.

15. An organic electroluminescent device comprising a first electrode; a second electrode; and at least one light-emitting layer between the first electrode and the second electrode, wherein the at least one light-emitting layer comprises the plurality of host materials according to claim 12.
